SEQUENCE LISTING (307 sequences)

SEQ ID NO: 1 - DNA, 588 bp, Homo sapiens
gggacagtggttctttcatttcaatgatcaaagttcccagctttttgacaccacaggggcaccctgacaattctggcaataagaacatgaaaggcctggtctttatttcactcaattcctgctatgtgtggtgagtgtgggtgagccaaggggaaggtgatcctattgtcaggaggtaatttaccatgaataggggatgatatggaaataatgtgtgtgatccttcccctgccactgttgggatgtctttttaatttccttccctcatttgtcacagccgtgaaaatactttttctgatatgatgaatgacagatggcagggtgccggcagcccttctggagggatgggaggttgtgtgtgtccacgataggggcccaataagtactggctgaatgagaaaatgaggagcctcactgtgggctttctttggggtgaatggaggtgctgagtgacctctcagcttcctagaagtcacaggccagaagccgtggaatctcagtggtggaaagtcctactgatttgaggatcagggagggagagaatcagcaatggtgtgctgataaatgtttagtagttggctctctggt

SEQ ID NO: 2 - DNA, 678 bp, Homo sapiens
atttcattaatgtttgattgaaagtaaattgaagtgtagctcaaggtggatcatacacatagcaacattattgcagaggaattattgccatttaggtaatagagcaatggaatcaaaataaaatactgattatatggattgatggagctttttaaatttaatgctgatttcaaaatgttttgatgattatttggcaagtgagtgtttgtatgttacgctaaaagaggattttcccccctaagatgcagctcaccataagaaaggttgtatactatttgtatatgaaatctggtctcccaacatcaactgagaaaataaataaccctatccttctgtaaacatggtatttactctctttgaggtattttcttgtctgaatttgaataccttgataaagtactagaacaaacaagtaaaatttctaaaattgacatcaattaatctatattcaaagcatgacaagaagaagaaaggtgatttattgaattgtaatcaagatataaggaataagtaactacaatataatttttccaccatatttagaacttaggagttgcactggttttgttggtgttttattgtacaaataatgtatttactctttaatatgccgatttatatttcctatgtttctaatggatatttaaatataacttaaaagaaacaagttcttttttc

SEQ ID NO: 3 - DNA, 567 bp, Homo sapiens
gccctgtgagaagagaagtcttttctctgaccagatgtcatctttccttttctaatacttcaggtcttatgccctgttgtatgagtggcatagttcattgatcttatcacaggaaatcagtgccttgagtatacgtatatggttgttgaaagaattcagttcagttcatgttatcagacatcataaatgaaaaatcttcagtgtcgtaaaggataggaagtgttaatttctcctttttactcttgtgacttttctagagggtccttatatattggggcaatttttaaattacaattaaaaaaatacctagcttaggctgggtgcgtcggctcaagcttgtaatcccagcactttgggaggccgaggtgggtggatcacttgaggtcagaagttcgagaccaggctggccatcacggtgaaaccctgtctccattaaaaatacaaaaattggccaggcgcggtggctcacgcctgtaatcccagcactttgggaggccaacatgggtggatcacgaggtcaggagatcgagaccatcctggctaacacggtgaaaacccatctctact

SEQ ID NO: 4 - DNA, 1026 bp, Homo sapiens
tgcattcacacatcccagtcacgatgacagtaaagtgtggcttgcaggctgtgctggggcctctcttcctttccaggcgtccctctttgccagcacctgctagtgggtgtgccaactccctcctgagcagcccagccccttgggcgccctccagcatgagctgggtcccccggcagcggttttaattatcagccctgctcaccccagctcctctcacaagctgccatatgtcatagactccagtaatcaccccgcagccggagtggcaggggaggggctgagggccttcaggggaatcctgctcagtcttgaccgagttcctcactgactgtacccgctctgacctctttgtctctggtggggcccagcctaggtacccacaatgggagagccgggcctagctgctttgggggcatagaatgcggcatgctctcaggcgccatggagtgtccttgggaaactgagagtcacccagcgagcccagggctgtggggctcatgtggtgcacacagttcccatgacccctcatggcctctacacgcctgccccttggaacgtggcatgtggcaggacagacaccccaaagctgtctgccagtctgtctaggagtccacgggagtggtcatttggcccccatcctcccctggtcactggccttgaggtaccacaggggacttcatcccagccactctggagggcatcttagtttccagccctctcaacctgccgtaatccttggatggcttttccagttggtgcctcacaggtgtgctcctgggaggcaggcggtgcaggagttcattatgatccccattccttgatgaggaaaacgaggctcagagaggataagagactcacccagttattggtagttctggagctaaaactcacttcaactgattttacttatttagttttccagggtaagtaacttctggttagctgaaagtaactttacacttgtaatgaaaaacatagttaataaagaacaggaaacgaaggttgcagtgagccgagatcacacca

SEQ ID NO: 5 - DNA, 474 bp, Homo sapiens
aaagcaaaacaaaacaaagacttaaaagatatatcaacttatgacatctgtgtgggccttatgtggatactgactcaacagacaaacgagtttaaaaattgtggaacagttggcaagttgaacatttgctgggtttgatgatagtaaggaaatattgtcaattattttttggtatggtaattgtattgtagttaatgttttaaaaagtagagagaggtattctttctaaggccgaaataacccctaccccaaaatttgacaggtgcatcacaagaaaatagaattacagtccagtaaacacacaaatagtaaataaaacattataagttaaaatttagatatatataaaaacaaggccgggcacagtggctcacacctgtaatcccagaactgtgggaggccaaggcgggcaaatctcctgaggtcaggagttcgagaccagcctgaccaacatggagaaaccccgtctctact

SEQ ID NO: 6 - DNA, 529 bp, Homo sapiens
gcctcccaggttcaagcaattctcctgtctcagcctccagagtagctgggattacaggcacctgccaccatgcccagttgattttttgtatttttagtagagatggggtttcactatgttggccaggctggtcttgaactcctgacctcgtgatccacccaccttggcctcccaaagtgctgggattacaggtgtgagccaccacgcctggacttttttttttgtatttttagtagagacgagcttttgctatgttgctcaggctagtctcaaactcctagcctcaagtgatctgtctgccttggcttcccaaaatggtaggattacaggtgcaagtcactatacctggcctcagtttctcatttttaaaaggtgataagtaataaacaaacataataaggattaatcaataaaaaataattatgtataagatgacatatgtgatcatatgtaataattatgtatatgttcaaccagtgaggttgcttctaccgagtaaacctgctggggccttggtgctccctaattc

SEQ ID NO: 7 - DNA, 454 bp, Homo sapiens
ctacaagtggtcaaagatctacctgtaactgtctagatatttgcctctaaataatgagacaatgcgaatgcaaagagccagtatgattaagaatatgaccattttcagaaaaagcatattgactctcttgggtcagatatggtggctcacacctataatcccagtactatgggaggctgaggctggagaatctcttgaggccaggagtttgagaacagcctgggcaacatggtgaaaccctgcctctctacaaaagtaaattaaataaatgaaaattttcacacagattaagagtttatttaaaaatatctttctcataaatactagttaatttcttttcacttatgaaattttttatagtaatttatacttttggttcaggcaagctgtgttcattttgatttaaagtaattcctataggtgttttgacttttctagactataagacctgtgt

SEQ ID NO: 8 - DNA, 247 bp, Homo sapiens
cagagggagaggggcatggcaaatcagaaagacagagcgggaggagagagagaaacagatgggcaaagcctcaagggaaactcattggagaggaaaaaagagagtctaggcacagtggctcaggaggccagactattcaagaggctgacgggagggggcatcgcatgagcccaggggtttgaggctgcagtgagctatgatcacaccactgcactccagcctgggcgacagagcaagaccctgttcc

SEQ ID NO: 9 - DNA, 254 bp, Homo sapiens
tggtccttgatgtcgatattcttaacactctttgatgggtaagaaaattaagactatcaaaggtaacagaaaagaagtaaatggcaactaaagcatggaaagtgagttttataaagaaagtaaaaaaaaaaaaaacaagtgcaaatatccatacttcaattgtgactcaaagccaacatgactctgtctacatttcagcatctcacttaagattcttgaagagggtaagctgatactcaagaagaattagtctt

SEQ ID NO: 10 - DNA, 3308 bp, Homo sapiens
ccctgcgctgtcgggcggggaggtcggaaaccccctggcgagaccacgggcggacgcttcccgaagagctgcctgggctgcagccgcggaagctgcgttctggggagcggggagcgtgctccggcgccttcgggccgctgctggaagccggaaccgagcccgggccgctgcccctcaccggacgccgcgcgccaccggccctccgcggggcaggggctgctgcgagctcgccgggcgccctttagacagtcgtccttgtctactccactaccaaatgttgaagttcttcaagaatcagtcctttggaggtgatgtcattgaaaatgatgagtaggaaactccaagagcgcatttctccacaaaaccagtgaatacattggcacaaattgtcagaatcaattttatataaattctggaaattagtcaaaggtttatagtaaccaaggaaacatctttttaaaaagatggctgagtggaccttcttttcaaagaattatggaggcttattttagttcccctaacttggaaatctcctgaggaagaaaggtgactacaggcatttgtcaaaaatttgtaaaggcaagtttattagcctctgccatcgggggcaaagaataatagctaaggcaaacaatagacacaccaaaaagcctgggaggaaaagctggaaagtaagatattttggagaataaaggcttttaaaacttccacatattcttgggaatccaaaaggccacatgtacatgcagggtgagcaaatagagaagacttgagaaagccttaaactctcacctctggctaaccatgaggcttgctcaaataggaagtgaaaactaaggtgaatttgttgcttagctgaatgttgaaggtgtgccccaacacttacacagagcctactggtaaagacagagtgttttctttttgtcttggtttcaggcatttaaggaaatctgtttctcttttggatcactagctgcaaattaagctaacagaacaggagctcagctggtcacacacagcaacgaatacagactttataaagttcagaaaagttaccaaacagtggtaaccataacaagtaccaacaatgaactatggggagggaggagaatctgatttccagagttaccacattataatactattcaaaatgtcacatttttagcaaagattacatgacaaggaaaaaccagaaaagtatggcccatacacaggtaaaaaaagaaattaatagaaactacccctgaagaagcacagacttcggatgtacaaaacaaagacttttcatcaactcttttagatatgctagaagagctaaaggaaaccatggacagagaacaaaaaaattaggaaagcaatgtctcatccaatacagaatatcaataaagagattgaaattgtagaaaagaaccaaatagaaattctggagttgaaaagtattataactaaaactgaaaattcactagaggtattcagcagcagactggagaagtcagaagaaagaatcaacaggcttcaagataggtcaattaagattatacagtctgaggagcagaaaggaaaaagaatgaagaaaaatgaacagagcataaaagacctctgggactctatcaagcataccagtatatgcatgaggggagtcccagaaggagaagaaagagagaaagggacataatatttgaagaaataatggtagaaaatgtcccagctttgatgaaatacatgaatctagatattcaagaggctcaaagaaccctaaatagggtaaactcaaaaagacccacaccggaatgcaaaagtgagctgggtgtggtggcacgtgcctgtggtcccagctactcgagaggctaaggcaggaaaatcgcttgaacccaggaggcagagattgcggtgagccgggattgcgccagtgcactccagctgggcgacagagcgagattccatctcgctattgctgcagtcattcagatggaaatggggaaagaataatattaactgatttcaaaaaggacttgaagatgtgaatcatctattttgctgaagaaatcttaactctttgaaattactttttgttgctgttgtcatactcttaggtgccaaactgcggtaaattttttatcagtgaagtggaagcatgtgttttgttgttttgggaatttttatcaagtatcttcagagaagattatttcctgctttatcttcaaaaactggaaaggaagggtcaaagaaaagacagtagctggccggtcatggtggctcatgcctgtaatcccaacactttgggaggctgaggtgggcagatcacctgaggttgggagttcgaggccagcctgaccaacgtggagaaatgccatctctactaaagatgcaaggattggccgggcatggtggcgcgtgcctgtgatcccagctgctcaggaggctgaggcaggagaatcgcttggacctgggaggtggaggttgcggtgagctgagatcacgccattgcactccagcctgggcaacaagcgaaactctgtctcaaaaaaaaaagaaaagacagtagcttatgttcatgtcaagcacctctcatcacagtctagttccaaggaaaaaattcccagcgttttctacattcggtgctgcgtcatctgaaatcggcacattccatggaggaaggagtcctgctttgttgcatgtatcctagggtttaatgttggtaaatgagtcactctagcatttgtagaaggctccctgagactcctgcagcagtcgaccaagcccaaggacataattgaatctggagagtcctggggccttgttttgaaaaagacttgaaatacacataggaagaaaggcataaaaataaatgttcacttgtctctgctgtgagtatgtgttccaacttttcagtgatggctttgagaattctcaaacttgactggctctaagtgtatctggtggcttttgtatcgtaacctgaaactggcttagtactttttcctaaaagctcaggatttgagaatgaggaccccttcgccaggaaaacatgtatacactcaaaattttgcttgcagttctagggtgtttagacctttctcagatacctgtgcatcttatgggttttgtttttctctttgagacagtctcaccctgttgcccaggctggagtgcagtggcatggtctcagctcattgcagcctccgcctcctgggttcaggtggttctgcctcagccccttgatcggctgggattgcatgcatgtgccaccatgcccggctgatttttgtatttttagtggagatggagacagagtttcaccatgttgg

SEQ ID NO: 11 - DNA, 755 bp, Homo sapiens
acatttcagttgggaacagattgctccatggtaatgtgatcactatgtacccaacaatggctctttcttcctagcgtcaatgcagatgttattttcaccttaactgttatcattgttgtttctaaccacatgaaagtgtatcctttatatatctgaagtaaattcatactagtggtgtaacatctccagccatttaagtgtaaaaacagaaaacgtatgatgtgtttacgtactgttttatactcctaacgcatgaagagaagatccttttattcattgcctatacttttatttctaaactttctgtaacactttatcttatatccagcatagaattaagatttgcttttcgatttaatctgacaatattttttcctctaataagagtcaagtccacttacttttaatgataagttgtgtttggttatattttgattacagtatattatgctatgatttatatgcacatatctgtcttttgctgtcttgtttgtttttattgcttttgttttgatgttgtgatatttggaagagttaaacttttattctgatggctaccttatgtaatttcataaaatcatctctttctttggacagtagctaatgtctctaaactaagaacaatggtattagctgtattctctttcttgtcctccctatgtgatttttcatcccacaatttgatttaatcatattaactttgtttcccctggtgccattaagtatgcttacatttctataaacaatatcctttg

SEQ ID NO: 12 - DNA, 393 bp, Homo sapiens
atagtcccattttatggatgtacatcttagtattcacgtagactcaagatgatttttatgcagatttctggagctctgtctcttgacagctttctcttctcttgtggtgctctctttctcaaattgtggttgccctcctaagttcctgtctctctcttaagcccagcaaaaccactgtactctgcttaggttctccttctttgtatatcagtcgatagagtgcctccaggcagaaggctggaactcagttcatttttctttcccaagggatcacagtcctcctgtactacctgttgttcagattttcaaagcggttactttatatattttgtctacttttactattttttatagcagatgctagtcccatattagttactccatcattgattc

SEQ ID NO: 13 - DNA, 359 bp, Homo sapiens
cgggagactagagatgagctgacgcaggaaaataaggcaacttccacaccaggaagaatcaaaagagggcgagcagaaaatgtgcaaagatcacccaggctttgcttcccacacgagcaattacaatgctcctgcttggaattctcaaccacaccagaagaccaacagatcaatttgagttactctttttaaggaaaaagtgacctacatttcatgaagcaaagagatacagccacacacaggagccgtttgttttaattagattgctggtttccctggccaggacccaaaaccactgtgtttccccatagatacaattgacaaataaaatacatgacactcatgtgaatcagaatttc

SEQ ID NO: 14 - DNA, 643 bp, Homo sapiens
gatattattaattcttaaaactgaatcctccatagaatcctaaaatttgtcatggactataacatatatcacatttaattttctcaaaggtcttgtagggtacataaaggagggactgcccctgattttacattaaattgcttattaggtgagagaatttttgtgggaccagaggaagaaatgcgttatatgtctcagtgctcttggcataattgtgtatgcagagtacatcttattttggtgatgtttttgtatgaaagacttttgagctcattgttatgactcagcaaaactatgggttgtattagttaatctgactcattccttaatggacataattattttacaagggtaaatactgtttctccatcaagactggttaaactattccatgtataaaggtcagctacatcagttttggttagaggtgtggacatttaaaataggtggattaaaataaagaatattccaaagataattgcccaaaatatccaaaccagtatttgcagctcaagtgtatacctgccgtgatggttatctgaacatcattttgtacctttgtttgcatttatttatgttttattttatattaaacatatgcagcccatgtaagtttcaaaacagttaataattctatcttctc

SEQ ID NO: 15 - DNA, 211 bp, Homo sapiens
gtagtagacatttttccatctcttacctttataaagtaaatatatataagaatgaagaattaaactaatagaattgtcgaattttatttcatttataatataagtaagcaaatagaccgagacaggttggttacacacttagtgacagaactaagactccatcctacaatcttctgttatagccacaggtaaaattaataactgccatcct

SEQ ID NO: 16 - DNA, 138 bp, Homo sapiens
ttgttttttgatcatttgcatcttcattataaaggaagtccagagaatgtatggctatgtcacattttgggcaatctctctgggctaactttctttaaaaggtcagattctcctggcaacagagagagactccgtctc

SEQ ID NO: 17 - DNA, 628 bp, Homo sapiens
caaattaatttaaaaagtaaacagagacagggtttgctgtcgcccaggctggagtgcagtggcgagatcatagctcgttccagcctcaaactcctcggcccaagagatctttccaccgtggcctctcaaaggcttgggattacaggggtgagccaccccacccaggccctgttattccatacattttccataaaattattttataatttttgttttgttttgtttttattttataaatttgtgtgtgtgtgtctcgctttgttgcccaggctggagtgcagtgacgcgatcttggctcgctgcaacctccacctcccaggttcaagtgatcagctcttgcctcagcctctggagtagttgggactacagagacatgccccaccgcaccggctaatttttgtatttttagtagaggcggggtttcaccatattggccaggctggtctcgaacccctgacttcaagagatccatccgcctcggcctcccaaagtgctgggattacaggcgttagctgccgcgccggccaaaattattccataaatttatccataaaaattccacataaattttctggagtttgattatgtattaggcttgttgggaaatttattacccttgtgaagaatt

SEQ ID NO: 18 - DNA, 403 bp, Homo sapiens
acgggttgatgggtgcaacaaaccaccatggcacatgtgtgtaacacatctatgtaacaaacctacatgttctgcacatgtatcccagaacttaaagcataatttttagaaaagtattcagctgaatgtgaatacagtcatgtgatctatgtcaatcctatggctttgttaacctgcagcaaattcacaatcacagaacaattaattgatcagatttaggcaaagtaactgcctcttaattattttggaggccaataacatcttttgacagagcatggtggctcacacctgtaatcccagcactttgggaggccgaggcaggcagatcacgaggtcaggagtttgagaccagcctggccaatatggtgaaaccccatctctactaaaaagacaaaaattagcc

SEQ ID NO: 19 - DNA, 582 bp, Homo sapiens
ggcctccagaaccaagagaagacaggggagtagggattctcccagggccccccaaagacaggaagagggggaaatgtattctcccggggtctccagaagcagccagccctgcccgcagtttggctttagctccctggtacccatctcggactctgacctacagaactgtaagagagtaaatttatctcattctgtgctgctcattgtgtggtcattggttacggcagccacagaaaacagacagtgcgcacatccgcatggtcccctctccagctcttgcctgataggcataaacgagggcagctgggcgcggtggctcacgcttgcaatcccagcactttgggaggccgaggcgggtggatcatgaggtcagaagattgaaactatcctggcccacatggtgaaaccccgtttctactaaaaatacaaaaaattagccaggcgtggtggcacgtgcctgtagtcccagctattcaggaggctgaggcatgagaatcgcttgaacctgggaggcaaaggttgcagtgagccaagatggagccactgcactccagcctgggcgacagagagagattctgtctc

SEQ ID NO: 20 - DNA, 317 bp, Homo sapiens
gaagtgcagtggtgtgatcacagctcattgcaaccttgaactcctgggctcaagtgatcctcctgcctcagcctcccgagtaagtgggatacaggcatgcactaccatccttggctaattttttttaaattttttgtagagaaatttttgtttctctaccaagtttttgttgcccaggctggtcttgaactcatggcctcaagcaatcctcccacctcagcctcataaagcaccaggattacaggcataagccactgtgcccgctctgtcttatctaactgggtaatcactcaataaaattaagttcttattttttc

SEQ ID NO: 21 - DNA, 269 bp, Homo sapiens
tccccatgagaagtgatggtggcctcgactgggagtcgggagtcatggatccagctcacattttcgttgaggaggaagggtggaggtggatgaaaagaggaggcaggtctcatattccaggaaggcaagaattaaaaaaaaaaaggaatgaaatgaaatgaaaagaggaggcagggtggtgtctaggtttacagcttagggacttgcgtgaattagggtatcttctactgtagtaggaagactaggggaggaacaggtcttggggagtt

SEQ ID NO: 22 - DNA, 354 bp, Homo sapiens
atactggacttcttccacgactctgtttacttcatcttatgtaaagtgcagatttactgcgcacaaggcatacatgattgagggttcctctaccctctcctttgcacatgcaacatttggattcagtgcacactaatcaaagactcacaagaaagtaaccgtttgtctcattttttctaccctcctcttttctccttcctctccagcccacttttccccctttaaatactgaagccctcaaaaccctctttggaaaaagtgcaggacacagatcctactgtggcttgtgtctctttttccctccctaaccagatgcatcctcaaccttagcaaaataaacctctaaattgattg

SEQ ID NO: 23 - DNA, 368 bp, Homo sapiens
attcctagaaaaatacaaactaccaaaactgactgaagaagaaatagatagcatgaatagaactataacaggaaattgatctagtattcaaaaactatgcacaagccaggcacggtggctcacacctgtaatcccagcactttaggaggctgaggcaggtggattgcctgagcccagaagagaccagcctgggtaacatggtgaaaccctgtctatacaaaaattaattgagtgtggtggcatacacctgtagtcccagctactcaggaggctgaggtaggaggatcatttgagtctgggaggtcgatgctgcagtgaactgtgattacaccactgcactccagcccgagtgacagagcagcacccca

SEQ ID NO: 24 - DNA, 459 bp, Homo sapiens
tccagagttctagaacaagtagatctagaacaattggatatccaaatgcaaaaatcccagacatatacctccaagcttatataaaaattattttaaaatggattatagaactaagtaactgtaaaatgtgaaacttacaaaagaaaacagaatatctgcacgaccttgggtttggtgtgttccctgaaagaaaacagtgataaattagactttaccaaaattaaaaattttgctctgtaaaagacagctttaagagaacaagataagccacagactggaagaaaatatttgcaaatcataaatttcataaaagatgtgaatccaaaagatataaagaactctcaaaactcagtaattagaaaacagttttttaaacgggcaaaacatttgagtagacagttcaccaaagaaaaagtgtgaatggtaaatataagcacatgaaaaaatatagctcattag

SEQ ID NO: 25 - DNA, 149 bp, Homo sapiens
gcaccatgtaataatagataaaatattaactgttataagttaatattgtatacatttatgtattaagcaaagtatacatctcaattccaaacataattttcagagtgaaaacgatacagtaactagtaaaacaatatgccgagaatcgt

SEQ ID NO: 26 - DNA, 90 bp, Homo sapiens
ggaatgctatcattttaaattattttggagctcattaaagtaagtctgcactggccaactttttatttattaattaaatttttgcctagc

SEQ ID NO: 27 - DNA, 408 bp, Homo sapiens
atgcccttgacctaaggcctctcctttcttttccttctctggggtgctgcctcatccttctggtcttcaaaaccgtttccctgggaaaacatctttgactcagcaggcagggatcatgcccctgctgtgtctgtgcataactttctgtggctacttctgtcttggtctgtgatgtactttataataattttggtctttcctccagtgtcacaatactggaagtctgtttctttttctctgtgttgtatccttagtgcctgaaaggtaggaggttctcaataaatatttgttaaataatcaagtaaatggagtctggtggaaaagagaaaaaataagtgtagaatgtgtgtgcaagaaaggaggggtagggggatgaaaaagataacaaaagcacataacaaaacaaca

SEQ ID NO: 28 - DNA, 697 bp, Homo sapiens
ttgcaaatatgttttgaaatatatttttggcttttgaattttcccttgagaattgtgtagagaagaatatacaaatcaaagaggatttaatatattattcattgcatatctttccttctgagattttgtttgttttaaatctttggaaagtatgttactcatttcagtatttccactgactttcactggtagatggttcttactaaattaatttcctgccatactatgttaaaaattttattctcaatagatattagccccatattgttttaaccaccattgctttatgttactaatctttttgatggtcctggaaagaactgattttaatttctatttattaatgaatttttgtttttacagttttaactcatgttacctaatcatagcataagaggactgttgcacagtgctcctgcatagagtacagcaacagtggctccatgcatgttacctgctgatgggatggatgctagctgagtgtttgagtagactaatcatgatagatatatttcctgttgtgtgccagacactgtttaggaactgatgatacagaaatatgccttcaggtacctgacaccctcgtggggaagcagacagccatcaattgtgtgatgtaatgtgtcactgtcacgaaaaaaagaagactgggaaaggggacagaggatgagggagttgctagttcatatgtcagtca

SEQ ID NO: 29 - DNA, 179 bp, Homo sapiens
agataacaacagagatatttttttcattttaacctgaaggaatgcagttaatatggttatagaaacaggtagattgatggcattggtgtttagaaatgagattatttttgtctctatagtatgaggctaggtcactagctatgattgaggtgagaatgggaaatgtgagaagtctgagg

SEQ ID NO: 30 - DNA, 277 bp, Homo sapiens
taatttgtggatagctatggcaagaatagatggcatgtggctgggcaggtggattacaaggttaggagtttgagaccagcctggccaacatggtgaaaccccgtctctactacaaacacaaaaaatttagccgggcgtggtggtgcatgctgtaatcccagctattcaggtggctgaggcagaattgcttgaacctgggaggtagaggttgcagtgagccgagatgacaccactgcactctagcctgggcgacagagtgagactctgtctcaaaatt

SEQ ID NO: 31 - DNA, 98 bp, Homo sapiens
atctttacacactgtgtgccctttaacacagatttatcttgactgatttatgcttttgctgtcttttaatcatagacaaagtaaaagcattctaaacc

SEQ ID NO: 32 - DNA, 241 bp, Homo sapiens
agacttaaccctaacatactaccaataatgacattaaatggaaattaaatggaataccaatcaaaagaggtggtaggggtagatttttttaaatcccccatttatatatctgtcagaaactcttcaaatataacaatataggcaagttgaacatcggaagatgtgaagagataacataacaaatattaaaaagaaagcagcatattggcaatgttaataccaattaaagtagacttcagag

SEQ ID NO: 33 - DNA, 1880 bp, Homo sapiens
agtagaatcaaaaatttttagagtcagtatactcatgtaagctaacataaatgagaaagagagagagcgagagaaagaaaggaaaggaggaagtgggaaggggaaaagaggggagaggagtggagggaggggaggggaggggagggagatactcttactcagaaattttctttctttgaaaatcccttatgacatttctaagaagaagcaagaatagtgtgacctttgcaaattaccttaaagacaaagaggagaagaaagagccaagctaatacatgaagagggaaaacaaccagaaaaaatgacatttcagacacaatcatggacagaaatcctacaagtcagtaggggccacctttacctgccagggggaccacaaaaataggggatttctgtcaagaaggcaggaatgttcagcagaacacagcttctgaatcatctgactctctcagaaccaagacaaaacagttcaaatgcctacaagccacaggacccaggaaataccgcagagtggacactttccccctctacataaaagaacctatttcttttctatgcatcagcttctccagtccatctttcattaaaaggacttgccatggaatgaaaactcatatttcaggactaagatggacaacaggccttctccagctcttctctgaaaagtgagcttttcggtagagaacgagcttccttcacaagaagggcactcccgctgggtgtgagccaaacgcacatgcacgacacttgcgcagctaagaatacgcacagtggggaaaaggcacagaagcagcccccgtcctgcccgagtgccacatccctttctgggctttcattcccccacccccaccgcctgcaaaatgaaagaaagattgcaataaacaaggtgtaagtctcaaacctgctcttcacctggagcttgtaatcaggtgtcaggctcccatccacccacaaggaacagagagattttggtgttgaagcttcaacctgccctgcgagccaatctttatttcaaagtactttgtgctgtaagctaacgggaaaaaatgatcaaatgcctcaaatctcccgtaagcagggactgtgcctggggggaaaggtgctcaccaaggtgggggcacatcgggtgtctcctggtgctttctgctggcactaacattctaaaacatgaagcattaagtacagcaacatggatcttccttttttaacatggaaaatacgttttcatagagcaggagggaaaagaactctctaaaaaacagagctgaataggcttagcaagaaaagaaattcaggagatggagaggaggagctctaaaacatccacaaaaaaataaaccatttcatagcaatgctgaccattttaattgattctcgacgacagaagaacacaagaaaaggtagatgatgtaatgcgatggctgctgaaggcaaaagtcacaaaacaaatttagcccttcgaataccacagtagccatgggtcaatataaaaagcttcaacggtcaggagcaaaactggggtgaaggggctactcccccatacatgtaatttgtccaagccctgccatagccaccacctccctggatcctcaaagcaaccctattatgcaagacatgctgatccaggtgcatctgacgattcagaaaaccaggaccaagccgtggggcaccgagcctgagctaataagcagcagagtcgaccctggcacgaaggtctcccagctccatgaagatgcatcatcaagaaggttgggcctcaaattctttccattacacttcatgtttctccctggattatctccataaaggagaaaaacaatacccagaacacaattccaactctgagaaattgtctgatcttcctccttgtctctgcccct

SEQ ID NO: 34 - DNA, 1199 bp, Homo sapiens
ctattccagtagtatatctgagtaaatcctgtccctcagtagatcatctcttgggatctggtttcttgatctgtatttcaatatattctatattccatatagatcaagactttctaacataaagcagtgtggaatagacttactttttatcttctctgttactcttttgatttgtgacttttaccaatttattgaacttcttaagtgtcagtgtttttaatccattaggttatcgccaaggcctctaaaagctctaagattcagtgatatgaatacatatttgcagtattagagacattgtactgttttcacttggcttctaggacattagattttctattctccctttcctatgctcactcccagattccttaaccagttccttgcatctttgtgtattagaatgcctcagggataagtcttggatttctgctcctttctagctgcactcacttccttggtaagctcatctgatttcatcataacttcacctttacatactgcaaactcacaaattatcttccctgaacttgagactcctatcctgctgcctgcttatcatctttacttgactatataacgaacatatcaaacataaactgaactgatagtctcctaacctgaaacctgcttctatagtcttccccaactaagttattggcaaatacgtccttgcattttctcaggccaaaatcacatcatgatccttggcatttctttctctggtaccccatgccctgtctgcagatctattggcaaaacctcccaacatcttaacagcagctttactaccacacttttccaaacggattacctctagcctgcatgattgcattagtctgcctccctgcttctggcttttacctactcaggctattcccagcacccagaatgacaactttgaaaacaaagcttgccgccacgtgcagtggctcatgcctgtaattccaacgctttagaaggcggaagtgggcagatcgcttgaggtcagaagtttgagaccagcctggccaacatggtgaaaccccatctctaccaaaaataaataaattagctgggcatggtggtgcatacctgtgatcccagctacttgggaggctgaggcaggagaatcgcttgaacctgggaggcggaagttgcagttagcagagatcatgccattgcactctagcctgggcgacggagtgagaccccatctc

SEQ ID NO: 35 - DNA, 336 bp, Homo sapiens
gtatgttaatgtatgtaatgcatagtatgagtatccagcattttaagcagatttaaaatggaaaaattcatgattcacattagagcttcaaacttataaaatttgggggatgcattatagcgtgagtattggcacccactcctgaagtggaatattggaagcctgaaatatatgacatgttgacagtaaagatccaggtaatattggccatgcggggtggctcacacctataatcccagcactttgggaggccaaagtgtgaggactgcttgagccagggaggttaagactgcagtgagccatgatcgtgccactgcactccagcctgagtgacag

SEQ ID NO: 36 - DNA, 700 bp, Homo sapiens
cttgttggcactgaggtaccggtttggaattcccgagcgtcgacggggggaaaaataagaggaatgaatattttaagctttgctatataattaaaatattcttagaagtctggagtctgtgaaggtcacaccctctggtcttctcccagcccatagggtataaataatctgaattgacggcatccagggatctcagaaattattagtacatcccacagtgaattaccaccttactaaaatattcatgggtatatactatggatttgttttatcctatttagtcttaaaaactataaagaaatctgcaggcttattaacatattactcagaatcatattgtctccaaagcacaaactgaatcagttacaagatattggactagagatcatggcaaatcagaggtacataagacctagttccgttgtggagctaaacaaactgcagagacctaaagggaagccttgcaccacactctaggtttggagctcaggttttgagtggtgtcagcactccagaacacatgggatccccgggaggtggaaattgagccgtctttggagaatcagctaatgagacagatgcatgttaaatgtctgttgtggcccaggcactctgctaggcagaggggtgaaccagaagaatgagattcatggggccaaagaatttgccttctggtgtaagaaaagatggaggcagcttg

SEQ ID NO: 37 - DNA, 855 bp, Homo sapiens
caacaaggtaggcccagggaaggggtttgtagggaggtggaataggataggggaagggaggaggcactgagcgacagtgaaatcaggacaggacgtggagaggatgaggtgtgtgggagagagcagaagggctttaattctgagacctgggattataaagccccaagaggggaggctgggaagtgccggccctcaaatgtccttactctgcacagacctagcaagggctctgcctgcccctggccgggtgtggacatggagaaggggagccaagaggtacgttcttgtgaggcgccttctcctcggagcccgtcccgcagatgtggactcacagccgcccacctggtccatgtgcctccgcagcctggaccggttccctcctctgcggggcggagaccagaacacagacttcctgagactgagtaataataggaaggatgtgatttccataatggaaataatggaacaaggaaatgatcctccttattattatctccaagggacagcgtgggaaaatacagcagcttctcctacctaataagaagaaaatgagtatataaaaatgtactgcagtttggcccaggggctcacgcctgtaatcccaacaccttgggagaccaaagtcgggggatagcttgagcccaggagttcgagaccatcctgggcaacatgtcaagaccccatctctacaaaagaaaaaaattttttttaattagccaggtgtggtggcacacctgtagtctgaactactcggaaggctgagctgggaggatcgcttgaacacgggagggagaggctgcagtgagccaagatcacaccactgtgctccagcctgggcgacagagcaagacactg

SEQ ID NO: 38 - DNA, 544 bp, Homo sapiens
gtgttgcatctgcagtgccactagaacaaggatagcagactgaggtggtagaaagcagactcaacagggcaaaaggcaagagatctgtttcaagtgcaagggccttgagccttttgtccagtggcaggatggggtggggtgagcaggagacaggtggctagtgtgataaagagtacggggccggttggagaagagtcattagaaaaagcctctctgaggaagtgacctttgagctgaaccagcacggggagagcacagagaagaactcagcaaatacacagaaagcacatatcacatgcaaaggccctggggctagagtgaatttgatgatcaagagacagtgagtagaggatgggtcagtaggtgtgcagcaaaccaccatggcacatgtatacctgtgtaacaaaacctacacgttctgcacatgtatcccagaacttaaagtggaagaaaaaaaaggggaaagaaggaaggaaggaaggagaaagaaagaaggaaggaaggaaacaaaggtaggtataatgacacggccgggggaaccctc

SEQ ID NO: 39 - DNA, 560 bp, Homo sapiens
tggctgaaaactttaaaagctcaggttagttcagatagattcagggtgagctgaaagccagccccctggccctgcggtgactttttccaaaagataaatgagtgaggccaggagtgtcatgcagacgggctttgggccggctatgggtgttggcattcttgttttgaaacccccttccacatctgctcaggggtcacaatcttaagtgctgaaggggtgcagctgacgaatgagaaaagcagacagtgtggagcctggggagctggtccttgcctcgtccttcaccatttgttgccctgtgggagtgctaagttagtgtttccagatcttctgattgttaagagaggctggaaatccgtatttttcaagaggattgagttgccaactcattgaaatcttctccaagccccttgcgagtcagcattggttagcatgtctcgaacacatggtagctcaaacacacacggtagcttgccatggtggcaatttcaaattgcattcattgatttcaaaagaccatcaatttcaaattgcattcatcttttgagttgcgaaataat

SEQ ID NO: 40 - DNA, 467 bp, Homo sapiens
caggaagaccctctcagaaaaaaaaaaaaaagaatttggccgttatgtggaggactggaattgagaagggcaagagcgaggtagaagagtggtctagggagaacagttaggggctattgcaattatccagcaagagatcttggaccaggatggcagcagtggaggtggtaaaatgtggttggatgaagcgtacgctttgaaggtatcaacaggaccagctgatggaagggagtcaacaggactagctgatggctgtaaactggggggtcactagctatcagatggcatttacttaaagccatggaagtaggtgagctcccttatggagagggaataggaaggaggtagaccattctatcaaaatgctctttctacagggcacttctcactgagatattatttatctgggatttatattatttattcaatttgttttgtgtttggttctattagaaaagctccatagg

SEQ ID NO: 41 - DNA, 1391 bp, Homo sapiens
gaaaattgtttttaagtaactttattgtataccaaaacaaagctcaaagaattttaacacaaaatgcaaaaaaatccagcacccaataagttacaatgctcaatgtctaaccccaaataaaataatgttaggaatgcagagaaacagaaaactgtaatccatgataagaagggggaaaaaaatcaatctacttaaactgacttagaaaagacacatcaggtgagaattaaaaaacaataaaaaggacacagatgagagaatctgtagataagcacattgaaacaaatataactgtataccttgtattaaagaagctaggccagtgtggtggctcatgcttgtaatcccagcactttgccaggccaatgtgggtcacatgaggctgatctcaaactcccaacctcaggtgatcctcccaaagtgctgggattacaggctcaagccaccaagcctggccaaaaaaaatttctaattgcaattctgaacaagttatgggttgtgaaatcaatatagtggactgcttgctactacaggctttatttaaatactaggaaggttggattacacataatgaaagattttttaaaaactgatcacaaagaattgtatatttctcactgcatcttgtggtcagataagtttgagaaacaaaaccatggcgagaggaaggaaaattccatcaatgggtggtgttaagccttttctatgaggtagctgtacatttgggacacttctatgttggcgacttgacattctaatagatagatggtccttttcatctctagccacatgtgaaaattacttgggagctttttaagactactagtggcttccacccacctggaagcatttaaatcagaatctctaggtgtagagtccaggcacttgtgttaaaacctcaccaggctttataatatgacagaatggtttaaagctactgagtagacccaccctatttcccaccattctctttgtttctctttcaccataggcttctttcccatgagaaagtaaagattttagtctctctttccacagtctgaagtaaatcactatctttctcaactggacttccaaggcaaagatttctttccatttatctatctggagttttacaaagttggcctctggattcccttttcccaaagctaattcaccacaaaggcacccctcaagtcaaggagctggactttcatacacctgcacctgtcaatcatgggtaaataatttgcaggcaaggttgctgggtgctgtgggattgacataaactcccaggtattgccagctctgagcctcaggcaagcttgtgactaaatgactccagtagtctgaggacagtccttactcagaagggtctttggaagcaaaagcagacataggcatgagagggt

SEQ ID NO: 42 - DNA, 593 bp, Homo sapiens
aataatagataaaccaatgccatgtgcctcctaatgacatgcactgagaaggatacatcattgctgtaatggtatttctgtttaaaatgtataacctgtatctaaaatgaggaaacatcagataaatccaaattgaggttattgagaacaatgattctaattaaatagtatgaaatagagaaaacgtaagtaaatactctatattcctgaattttaattggtgggagtatcactttgaccagtccagcagcaatacacatcactagcacatacattatggtatttatggaccatttcctgctaaaagaaaccaggatttcttgggagaagtggctgattccaagtatgggcagaaaatttttaatgagcctgcagtattttctcataccagataataacaaagctaatttaaaaaatcagtagattaatgacaaagcactgccaacttggaaaggtttccaatgaccaaggataggacaaatcaagcttaaatataaaaataatttatatttgaaacacaccaaatacatttatagttgaataaatacaaatttacatttatagttgaataaatataaatctacatttatagt

SEQ ID NO: 43 - DNA, 767 bp, Homo sapiens
gaaatgacttccataaggttgtgcagctagtttgcaacaggtcccctgacttccaggcccgtggtgtttctgttacctcccagtggttacttgcctgcagctagaagggctttctgcagtgctgctgctggagttggggggaaaaggctgacactcagcacagccttctgcatccacttgagtcatgcaggacacttagctttgttctttctccacagttaatattatgccaaacctacctgtaattagtaattttcaaagaatattataagttccagtaaccaaatgtttgggcataattatatgccaaaagactactttttaattgataatttttaactgctttttatatatttgcagcctgagaaggctgtttggatactgaggttcagcaaagtgggtctgaagatacttgtttatgcaaatgggactttgtaacctgggaaatctacaggatttatacaaattattattgaaataggcttaactgtccgggcacggcagctcatgcctgtaatcctagcactttgggaggccaaggtggatggattgcttgagcccaggagttcaagaccagcctgggcaacatggtgaaaccctgtctctacaaaaaatacaaaaattagtcaggcgtgatggtgcatgcctgtggttccagctactctggagactgaggtgggaggatcactggagcccagggagttagggctgtagtgagccaaaatcatgccactgcactccagcatgggcaacagagtaagactctg

SEQ ID NO: 44 - DNA, 1145 bp, Homo sapiens
gctttgaacagcttccccttccatctgtaactattgggtgaggtggaattaattttaatttgttctacatgctgaccagttgcccctctgtttactgaattattatgtcttctccattgagtttgaaatgccatttaattatatgttgtgtatgtgtatttatacgtatatgtttatcagctctctgtcattgatttttcttcttgcacacatagtataacattttaattactgtacctttataccaggtcttggcaaacaatggcccatgggcgaaatccagccctaccacctggtttttataaataaagctttattggaaagcagccatacttacgtattgtttatggctgcttttgagctactatggcagtgtagttgcaacagagactgtatgggccagaaatccagaaatatttacaatctggcccttcatacagagtttaccaggctctgctttatactgtgtattgatatctgatagggcaagttcatcctcattctttttcaacaatttcttggcaggcctaacatgtttatttctccagatgaactttagaatcaatctgccaagcttgcctgacttccttcttttccccacctctttttggggtggagaactggggagccagcagaataggaattttgattgcattaatttgtggtttagtgaagggagaagtgattgctttacaacgttgggtctttctattccagaaatatctctttacgtgtatatctttcagtaaactttaattgttcattctgaacaataaaatcatacatattggagtttattcctatatgtattgttgctttttgttgccattataattgcgttcttgtcccagttatattttgcaagtgactatggtataaagggaagtttttgctttttatgtatttaaattctgtttctaaccctcttatgagagtaaactattaggactgttaatttttgtttcttttgattgagagtcattgtctgaacttaccaataattgttttattaatgtttatgtctccccctgtattgtgtagttttcttacctagagtagtttgggggaatggactttgaccccctcaatggcattcatttttttttcttttgtgtaggtcacagcaaatggtagttaaaacaagc

SEQ ID NO: 45 - DNA, 338 bp, Homo sapiens
ggaaagaaaatgtagaaataacagagatcaaacaaaaaaacaaaaacggcagacttaaccctaacatactaccaataatgacattaaatggaaattaaatggagtaccaatcaaaagaggtggtaggggtagatttttttaaatcccccatttatatatctgtcagaaactcttcaaatataacaatataggcaagttgaacatcggaagatgtgaagagataacataacaaatattaaaaagaaagcagcatattggcaatgttaataccaattaaagtagacttcagagcaaagaaaattaccatgaacatagaggaatattacataatgataaga

SEQ ID NO: 46 - DNA, 440 bp, Homo sapiens
aatgatggatcattggtgataaatacacaaaaacccaaccaaacaaagacagttactccaggaataacaaaaatgtgtgcaggaaaggaaaaggattccaagtacacaaggaactcagctgcccctatagcacttagaaagtcatgataaagtcaacagtgaacacagagttaaaactctgtggggacaggggaaaatatttgtcatgggaagtgaggggatatttgagtaagtgaatgttggatctttatcttccataatggcaggttcataacaatggctacaaactatagcagttaaaagaattagccggggcccggtgtggtggcttacacccataatcttagcactctaggaggccaaggcaggcagatcactcgaggtccggagttcaagaccagcctggccaacatggtgaaacctgtctctactaaaaatac

SEQ ID NO: 47 - DNA, 1098 bp, Homo sapiens
aacccctctccccagaggatgccttgcctggtgaggtcaaagtacaagatggtgccagttactgagatttggccgaaatggtcttggggtagtcgcgggagtttggaagtgggggtaaggttgctggaaggtttcaaggtctctcatctgctccctctccgtttcccatgaaatgcccttgtttaacgggctgtggtgccgaactccgggatcactcccacagcctggaagggagccgttgcctccagctgcagtgcatcaagggagctcggaatagaccctgccctctgtcagctgcaccagtggctgtccatggggggagaggcagaagcctaccagaatttcctgtcttggctccccagatcagaatcaagggacttctggcctctggactgaggaagtgacattctgtttttcaaaggaagtgttgttgttgcggagtacaagtgtgtgtcaatgaaatcaggctcttaggtagatgtttgctgggggaaaaaaatctaaggatttagcacatgagttttgaaagtggacgtggatttataggaggaatgaagcagtgaattgttcatctcagttcggaagctcatttttaggagtgtctatgtagccaaagtataattattaagaaataaacttttttcctcttcagggttgtatcagttcgttaggaaggttgaatattttaattaggattaaggagcagtgatttactattaacaattttataaataatttaaaaactttgtcccgaagagcttccaaaaattatctatacaaatagatttccatacaagctagtggaatacagtgtccacagtaaaaaaaaaaaaaaaaaaagtgacccttaattttcaagtttgaacactatacactaaagaaccttgaaagttgtttttgaaacaatttgcaaacagtatgacactgtatctacatttgacttatcgctccttgaactctcacccagactctatgacccatttcttgggtgtttttgttcccaaacaactttagttcaaaataaccaggtttggaggcatttgggtcaagcacctttttcactgatttgaacgaatctagtcgtatgatggccttagc

SEQ ID NO: 48 - DNA, 1477 bp, Homo sapiens
gtgcaatggcacaatcttgactcaccacaacctccgcctcccgggtttaagcgattctcctgcctcagcctcccaagaagctgggattacaggtgcacgccaccacgcccagctaattttgtatttttagcacagacggggtttctccatgttggtcaggctggtctcaaactcctgacctcaggtgatccgcccaccttgggctcccaaaatgctgggattacaggcatgagccaccgcacccggctggggtttctttgtatcttttatttattgaacctttgttttttgagtgttcatagtttcttgttaaaagtgtttttgtttgttttttaatgatagctgctttaaaaatccttgccagacaatcccaacatcagtaccatcttggtactggcatctgttgattgcctgttctcattctggttgactttttctgttttctgacatgacaagtaatattcaatattatcagtactttgggtattatgaaactctgattcctttttatattttctactttagcatgcattcaacctgcttcaattcagaatgcacatcatgactcacttctgtggtctgtgagtttgaatgtcagtttggtttcaaattcagcgttatcttggtctgctctgcctgtgtgctacccagagaccagtggatacccagaaacccgagtggtattccacagcatagctcagttcttaaagcttttgctgtgttaattctgatgagtttcacacataggccacttggggatgtgcacaaattgaaagacgctttttccgcagctccctcctctctgttattctgcccacactctctgtgagggggtaggtgctgcctctgttactgcaggacaggtggtagtcaacagggctctaccctagagtgtccatagcatcccatgggaagaaggaggagggagggggtgtcacctcttatcccattagtgcaggatggggctcattaatagagctccacttgtctccagaatcactggtgaggaaggggagtgttgcccccacattcgtgcacagcagggatggttcaccgaactccacaccagtctctgcagagcctgttggggagaggagggctgtggtttctttgatggtgttcacctggagtagagcaagtattgtcaaaagggtcatcctcggaggttgcagtgagccgagatcgcaccattgcactgcagcctgggagacagagcaagactccatctcaaaaaaaaaaaaaaaaaaggccatccttcattactgtcctcttctaggtcctttgactagagaaagcattttccttaggacttttgttgtctgtgcttgttggtcatttcagattgtgccttcctctagtgcccaggttgaaatacgtgaacactacgaaagcctctgggaactccctgccaggtcatcccttgagacttaagtttccttgccagtctgcctagtttacttcacctttagaggtttct

SEQ ID NO: 49 - DNA, 619 bp, Homo sapiens
cttaaaatgataccacttcatagttaataccagcaaactgcttagtcaaaccatactatgcggccctccacccaagagcattgtttgtgcaggtaggatctgcagtgtggatggagggtaatggaaaattgtggccactgttagctggtcagactgatatttactgtatgtcaggtactgtgctgagtccttcatgggtatcatttcgtttggtccttgcaataaccctatagggcaggtcctattattagatgcattttttagctggaggtgatcacactgctgaaaagtgacaaccagattcaaatgcagagtttctgactactgtgatatagggtcccggatggcacgtgcttaccagcagccaaagagagtccatttggccttggaatttctaactcagagactgaaacacagagggacttgtaggtggaaccaaggtttaaatgacttaattggatgggcttacctttggaggaacaccatagaagcaaatgtgtttttcaaagatcttccactagatgtcaccaaaaggactgagaactagagaaaagggctgcgatttctgctctccttgtaagattgcacaaaagaataaattgcatttatcgctgtttgc

SEQ ID NO: 50 - DNA, 789 bp, Homo sapiens
acattgtatgtgtgcctctaggagggtcactgagatttatgataaacatatatattgattgtccaagaaaaggtgaagaaacattaaccataagtcacaattccatgaacacatttaaaagtaattagtaaatgtgcagagacactgttaggggagtggatgttactactgtcatttatgaaggatttgctagagatggtagatttcacctgttgtgaattggaggaggagcatggctggcaattcgaaaggaggtaatctctctggggtacaatggagtagaaaacttagggacagaaggaatatacgaatggagaaattcgatttgcccaatctttattgctcacctattaaagtgctaaacaagctgatggtgattcctgttctcagaagcctgtgttctagcaggttataagaagatgagtctggttaaagagaagagcagggaagtggcttagattatggcataaactgaagttgaaactcagaatgaaaagtaggagtttgctgaggggaaagcaatatataaagtgatttgtgctataggacataagacagattatagataagagaactcagaaatagtaaggacagtggtaaaaagttaaaggatcctccctttccccagttaaccaggagaccaaataagggacttggtggtaggagtggtaggagcaggatcaatcacttatttattaagcacctgcacatgattcaaagaaggataagacggcccctacccttaaggagtttatgttctttctagttgtgaatagagaaagcatatgc

SEQ ID NO: 51 - DNA, 1530 bp, Homo sapiens
gagacggagtttcgctcttatcgtccaggctggagtgagtgtagtggcttgatctcagctcactgcaacctttgcctcccgggctcaagcgattctcctgcctcagcctcccaagtagctgggattacaagcatgtgccaccatgcccagctaattttctgtatttttagtagagacggggtttcaccatgttggccaggctggtctcgaactcctgacctcaagtgatctgcccgcctcagcttcccaaaatgctggaattacaggcatgagccatcacgcctagcctactctctgaatttctaaaagtcagtaggttgaccaaaaagtctagaaactggctttaagtcagtatgggacgtacttataaagagtccatggttttgcacgtttcggtagacaagtaaatctgagttatttttcaatgacttaccaatatttgaatagtaactaagatcgtcagtgtatctggacttctttttttgaagttctaaaacaattatagtagggatttattattttgggcctccatccagatgtttttccaagatcatttttaaaattcatttgtcttctgtttccagataacatactttccgttctataggaatcttcactgccaatcatagtatctaccagtggctttcttagactattcactccaaagctgggactgatgtcctgccagtagagaatctacagaaataatttgaatgaattaaaaccaaatcttgatagcaggagacagcttcctgatctagatgtacaattagagtttaggttggaaattactttaaaatgtgttttttggggatgtcttcaatctctgtgtaaatacccacatgcttatgcattgtaaaccaagtgtgtattcctgtgtatgaatttgtagaactgatttctgcttcaagagaagctgcacctttaattttataaggtcccctccacctgtaaccctataaatgtctgtaaataaaacactaaaatttgtagtgataggatcaatttgggaatatctgctgagagaccaaaaagttcatttttttaagtaccttggttaaagagtaaagattattcctcttattttttaaagaagaatgcactttaacaaacatagagctgcatgggcaattcaaacaaatctgtgaagtgcagtacccattcagaaatcacacttcctgaaaaccgttcaaaagcagagtccagacgggctgttgatctcactgcctgtaggttgaagctcagattctgatcaattttgagaggagcagggctgcttcaaaagagcaatgtgaatacagtcagaagcttcagactggtctgtaaaaatggcgggtcccgtatttaccactaactagcaaaactgacagaaaaactcacagagaaaaaatgtaagaatccttcctgctggtgtgcactccttacaatagacttttgcaaatggagttttacagtctatatttaaaaaaaattgtatgtttgtaacaaataaagtatgcagaaaagtgaatgacaatcttgtgcttgtgt

SEQ ID NO: 52 - DNA, 1310 bp, Homo sapiens
gaatcgattgaaatagtatatgaagtggtttgaaaataggtacaaactattgacatttcaatgtcagagagtgatacctgtagtagtataggcaaaggtccaaccccatcgaaaggcttaaacatttaccttttctgaaaaactattgaaatataaagagagtccccagtcacaggggcaacttctgtaaccaaatccagatctgaggaaactcctgtaaccccatttggggtttctttctaagccaatagggttacaggttggtacagtgacacattgagaatggggctacaaatacttttcccaccatctaggatgaaatacacgaaatcctgttgaaatcttggtttttatgcctttgctcatcagaataaacgtaaatgctgaaaaacaaataacctcctgatccactgtcttgcctcctggtgagaaatgattctatcccctgtttattgggaaatttccaaagttgttcatcacttaaatgccgtattcaaagggaacatggaaggatgaagcggagaaagtgccttcgagacattcacacatttctctggactcagtctgttaacatatcagggagcttgtcagatcacacctttttgccttggaaatcctacagatttcctgtacgccttcatatctgattcttccctaaaacctttgggtatgatttcctccctggtcttgataatgtcctgcagtctgtgttttataattattctttgtatttattgaatctagactttaagttattcagagatcagaccagaaccttagagtttctaaactgtatgtggatattaaataatattaataatgaaagagctaccaaaatagtctatattgtgtgaacaatctcttgggatattagacgtgtttaaagaccagtgttgctgctatttttaatattttggttaatttaagtgaaatgtacatattttaatttgaagatttatcttgcccatcagaatgtgaagatatacttgcatatattttgacatatttcatggaaaataaaaatgataatccactttgtgagtgtaagtgaatgtattcatatgtatgttattataaatgatttttgtttgcactgatgatgaaatgagagttttgggggctttttatacatttatatcgactggtctctaaatctcctattttgttttcttatcatttttgaaatacagttcccattacatgagttttaaatagattggtgtttcattttgtattatgctactactagatgttgattctctggtattgtaaaataaaatgtgctccaaaaacccaaaaaaaaaaaaaaa

SEQ ID NO: 53 - DNA, 2538 bp, Homo sapiens
agaaacttcactgctatttccagatgtcattttaaaatattttagaatacctgatttctccatgacctatccatgcttttctaaggttccaaactaaaatgcagaatcttgagttattccagaacatagatttaaaatttgatcagaaaataaccttcatttaagaaatgaggggtcaggcgtgagccaccacgcctggccaccaatttttattatatgattttataactaaaatttcataactagctaatgaaattcttcttctctcttttttgtttatttatcttccttttagtctttctttctcctcggatctttccccttctatctgtctcagttccttcattttccttagctctccatttctcccagcatctgctactagtctagtctcctggctcttaacctttttgagacacagactcctttaataaagtgatgaagaaagttatctccccagaagaatacacacagagaacacagaatattttgcgtattatttcaaaggtaaagaatgccaagaagccaggggcagtagttcatgcctgtgatcccagtgctttgggaggctgaggtggaagaatcacttgagcccaggagttcgaggctggcctgggcaacatggtgagacctcctctctacaaaaaaattttaaaattagccaggtgtgctggcacgtgcctgtggtcccagctactcaggaggctgaggtgggtggattgcttgagctcaggaggtgaaggctgcagtgagccatgattgtgccactgcacttcagcctgggtgacagaatgagaccctagctctaaaaaacaaaggatgccaagtatctaaactttgagctccttgaggacaaaaactaggcgtttttcatcctatatgcccagtatttagttgatgtttcttgagtgtatataagtgtgcacatgcccagaaacatgtaaatattagtacatgttgtagaaaagctgttgtcaggaagatatttgtacactctggctttccactatgatagtcaccaggcacatgtgggtactgagcactggaaatgtggattgtccagattggaatgtactaattgtaaaatacgcactggattgcacaggcttggggcagtacaaacaaaagaatgaagatatctcattaatagtttttatgattattacacattaaaatgatcatatcttggatatattgagttaaaatatattattaaattaattttacctctttattgttacttttctaaaagcagctactagaaaattttaaattatacatgtaactgctcatagaaggttggtatctgggttcattcattagtggacattcataaacatagtaattttctttaatttcatggattcgttgaactaaagatcccataggtcaccgccttccctgtccctcctctaccaccaaaaacttaatgagaacaaatgggaagaatttactctgcttttcaaggtactctgatacagatttttatctactgtcataagtatacctagaacaaaagcactgttgactcaagtagtttcactaatgaaaaggaagcagcagaatgactaatgtaaattggaggagactcttttatttggaatgctttggttcttccactgtggaacaggtgtggctgctgttgaaacagcagagtcatactaggcatatctgacatgtgaggaaccgcagcattgctcaggggcccctgccttccaatgaatggatgtaggatccatcatacatcagattgctcctttccaatacaaactctgatgcagaaatgcacttggtgtatttgctttttcttactttctggtttagggcagaaataatattttggcttggagacttttgtcctgaactatgacataataggatgagaatatcgtgtcaaaaatagccttacaaggtcctttttggcattaagacttctggagtgagtttgcagtggattattgagaataattctgttcattagcagctagccatctttgatgagtgctgacttctctcctttcagcacagagcaggaaatgcctgcctcccatgactctgggttggagtgaaggggaatgcataccagccaccctcttgcagaggtggggcaggtgctggcacagagcctcaggttaggccgaggggatgcaatctcagatcagcagccagcagtgtttgtaaacaacaggagggagattgtgctggtgatgtccaactcacaccaatgaagatcaaccggtttgtgctttgggcagcaggctgcagatggacagtgcctcctgagggcatcgccatgttttagggatccgtgttgcaggatacctgtctgcaagagagagtcaaggagggctttttaagcccctggggttcaggcctggcatctgggtgttaagtagagtgaatctcctgaagtccaaactaacatatgacattttaaaatgaggaaaacaaatggctctgaaaaggtctataggattataggtaagtggttaatacggaagatgttataaaggtctcaggaggagatggggtgatcca

SEQ ID NO: 54 - DNA, 763 bp, Homo sapiens
aaaattgtcaatgtggatgattctttaaaccataatttgggccaaaagctgagcatcacaccaagaaaatatctctgcttctagacatcaagaaagagaggtggagataaaggaaaaaacttaatcccgaattgataggagtgagagacaacaaaccttaggacagggaattcttaacttgtggcagagcaaacagtagaaactcatgagacgtgttatccaataatagaaaataggaacatgagatttattccactagacagtactaggactctacatgtaaactcatgggaattgaaataaagttctctgctgtaattggagcaagatagactgaggagagagtaaaccacgaatgctggctcaagacaaaaaacctagcagaggtgcattgcagacatacccatgaaggaaaaacttacacaaggtcaccctaaaggaaggacattgttaagccctttgaaataatggggtggagaggaaaatgaactgaaaaaatgaaaaacacccacaggaagaaatcaaagacgattgtgtcaaccccagggctacagaagtgaggaataaaattggctatttccggacactgactttcttgatttgttgaacatacgtgaaagcaggacatgccatggtcgctggttgcatcaaatagaaatgactcattggaatgttacctccaaatccttacatgaagagtaagcaaaagatgaaagcttttatgattcctttagaaaagaattgcttttgggactttatcata

SEQ ID NO: 55 - DNA, 934 bp, Homo sapiens
ctccgccagacagaggtgctggggctgtgcaggaaacgaagtgattagaaatcccggaaaaacacacaagcaggcgttgtcatggtgactgggaaaaacacacaagctggcgttgtcatggtaatggagtgtaggacaggcctggagcccctcggtctcttgctggcggctggcacagagacgggctgccgtgggctctgaccttaataccgggtcacagtcgcttctaggaccaagaggacagagaccccatcaccgtatgcaggggcctgtttccaggcagactgcccagtgcccagctgagcctcgggtgcagtgcgacccccgcagggcatgtccagaccccaggaccccctctcaggtctagaagatccagttgggcagtgttggtaccaccaagagtagacaggacagaggatcagagacaatcccacccagcaggacccaaggactcaggcagtggcttttcaggtgtgtgggccgaggactggggagtcggtgaattctggggcccctggggtggccgttcaggaactgcagcagctccccccaccacagatgctcgctgcctactgaagcggccacgtgtttgaatgaagagcagttagaggaacgcttgcaagagaatgtgtttattacctgaggttatgacaatacagaacatacaatgttttctgtggaaaatgtgatactacagaggaaaaggtcactttaattaaatggcaattagaagtaacagcattgcaaggtggggtgcagcagctcacgcttataatcccagcactttaggaggctgaggcgggtggatcacttgaggtcaggagttcaagaccagcctgggcaacatggtgaaacctcgtctctactaaaaatatagaaattagccacgcgtggtggtgcgcgcctgtagtccaagatactcaggaggctgagg

SEQ ID NO: 56 - DNA, 838 bp, Homo sapiens
cccactttctcaaagtttctctctttagtcactttgtattagattcatccattttaaaaatctttgctttagaagcattgttaatgtttttgtccatttcactagagtccctgaggaacatcatcttgggtttaacagtattaattgaccacccactatgtagccagctatgtgctaaatgctgaaaaaaataagaatacgttgcaaccctgtcattgaggaggcatattagttagatttctgctgtgacaatattgcatatcacacaatcccaaaatctcagtggcttacaattgcaaacatttatttcatgttcatgggtgtgcaggttggctgtggttcagctgtgtcactaggctgaacttactcaataagccacataacttcgagtcaggttccagtccattgtatgtgttattttcaaaatctaggctaaaggaggaacagtcatgtgggtcctactcttcctatggtggaaggtttaagcttaaaagggttggtgattattatgccttaaagtcttagctcaacagtggtacagtgcaatgtcttccatttctgttaccaaagcgagtcacaggaccaagcccaaagtcaatgacattagtcaatgtactcttcctggtaggaggtcttgcaaaggtcatgttgcaaagagtgaggatatataatattactagagggaggaggtgcctaattgggaagaataatccagtctaggctgcgcacagtggctgaagcttggaaacccagtgctttgggaggctgaagtgggaggagatcgcttaaggccagaagttcgagaccagcctgggcaacctagttgagaccctagccc

SEQ ID NO: 57 - DNA, 1319 bp, Homo sapiens
caggcatgagccaatatgaccagctcaaacatcttctttttaaatgtcagaagcatgtatagtgattatttcttattttttcccccttgatccatctcaccagatgtttgttgattttataagaattttcaaactaccagcttctggctttgttgaacttggatttctgtttcactaattttctttctcctgtctttgtacttactttgttgctctttttctaagttttaaagatggatgccaatctcaggcttcttttcgtgtgtgtatgtgcgtatgtccataaattctcttctaattacagtgtaagccgcatcccacaagttttgatagtcacagaactgtatcgtcacactattttttaatttcagtaagttcttcactgatccctgtgtaatttagaaatgtttcataatttccctacattggaggggaagatagttttgtttttattattaatttctagctgtattgagctcttgtcagagaatatggtttattttagtcgtttgaaatttaagatctgcttaatggcaaaatgtatggtcagtttttgtaaatgttgccagtaagcttgcgaatcatatgtactctagttttgaaatccattgctcagtggatgttcattaggccaatttgtataatcatgttgtacaaatctattctattcttaactgttttttgttttaaaggtgtggggtcttactatgttgcccgggctggactcaaattcctcagcctcccaagtatctagaactacaggcacgtgcagcttggtttaaaaaaaaaaaaaaaaatcagtgagaagaggatttgttgatctccccgttaggattatgggtttgtctgttcctccttctcagcttatgctgtatatattttggggctgtgttattaggtgcatccaagtgtatagttgttatagttaccatgtgagctcaaccttggatctttacatagagattctctgtatttagtaatgttttgttcttaaaatctgcttccatctaacattaatataaatgtaccagctttattttatatgtatgtttcttggactttgtctttatgtattacaagaaattgtgataaagacctcatttaactggattgtgaaaggactaggccattctgggtcatttacttttctgaaaaatatttttattttcttggtatttaaaaaaaggtttataagacattctaatttatcttagttttcttccttcatttatttaggggtctggtatcttagggatatcattctgaaaattaaacttttctacataggaccatagatacagggtgactagatgactggg

SEQ ID NO: 58 - DNA, 709 bp, Homo sapiens
ggcttggaggtatgggtaagcagggagacaaagggtacaacacttcagatgcaagaaatgagttctggtaagccactgcacagcatggtgactacagttcataaaaacgtgagacacagtggcatgcatccatagtcccagctgagaggctaagggcaagaagatcacttaagcccaggagttcaagtccagcctgagcaacatagggagaccctgtgtctactaaatatacaaaaattagctggagatggtggcaggctcctgtagtcccagctacacaggaggctgaggcaggagaatcgcttgaacctgggaggcagaggttgcagtgagctaagatcgtgccattgcactttagtctgggcaacaagagcaagactccgtctcaaaaaaaaaaaaaaaaaaaaagcccacaaaaaccagcaaaaaatcctctgccccatcaccccagttgcctcaccaacagcctctcccagaccaggaagctgtttttattttaacttcatgcaaatgttgctaatacaagatatattcatttttttaacttacccttttttacaaaaaagatggttctgaaattgaactgtatttaatgtctttaatggtgaaaaaaggaaaagtcatagatgacatgtcattattttgtaaaataataagatcatggtctggtactcactttggcagcacatataataaaattggaaagatc

SEQ ID NO: 59 - DNA, 1187 bp, Homo sapiens
acaactataatttgacttcggaataaaatttctttcatcagaaatgtatgttttgataggtgcactgcataggattctaatagctctaaaatctgcttcaattcagagctgtgatcttcatcacccctaagccttatatcttactctccacaaattagactgcatccttaaaaggcatccgctgacagatttcacagggactgaagtgggctgggaactgcattccatggcatctgagcttcccttagacaggccaacttcgtcattcagagcaagcactgaataaatctcctccaacttacatgaatgtaacccacttcatgactgtcagagggaagaaataagcctttgagaatcctctgttctaacagggctcccctcatgataatgcctagaccggtggccagagttcccacagccgaggctccaggtacagatgctaaatgctggcccagagggtcagcaggatgagctagtttctaagtgaaagactctcattacgcaaatgagtgcttagggccttaacactaaccaattcacacaggtctgacggggcatgagtgtgcaagtgaaagccatgcaggttcctgagacagccacagtcggtggggatccatcaggggccggcctcaatcccagcattttgggatttgttacgcttgtatgttctatgcattatgtacagtattcttaccaacaagtaagctagagaaaagacatgctattcagaaaatcaaaaggaagcgaaaatatatttagcattcattcagtggaagcggatgatcgtaaaggtcttcatcctctcatcttcatgctgagtaggtgaggaggaggaggaggagttggtcttgctgtctcgtgggtggcagaggcaaagaaaagccacgtataagtgactcacacacttcaaattcgtgttgttcaagggtcaactgtagttgtttttaagatgcttcacatctgcttctaagtctgctccatccccattccccagctaacacaacctttctaagtcagtcctgcatgcactctgcactcttcccaggttattttgtctctcaatgttacaaccaacaggctcaagcaaagcaggaggatggcttgagcccaggaagtggaggctgcagtgagccatgatgatcctgccaaagcactccagcccgggcaacagaacaagaacctatctc

SEQ ID NO: 60 - DNA, 408 bp, Homo sapiens
tgattctctcacagtctggaggctaaatgtcaaaatcaaggtgtcagcacaacatgctctcactgagaccgttaggagaatccttccttgcctcttcctaccttccgatagtggctggaagtccttggtcttcctctccttatagataaatcactccaattatttcttctgttgtcatgtagccatctgtcttcgtgtggtgttctcatatcttataaggacaccgatcatattggattaaggcccattcctatttccaagtaaggtgacattaaaagatactgggggttagggcttcaacacatgaatttgggaaagggggtatgcacaattcaacccataacaccaactgtaataaattctatattgttgtaaaatatctctttggtagcaaagttagtatatgcc

SEQ ID NO: 61 - DNA, 2907 bp, Homo sapiens
gtacctttgtccttggactttggtgatgtggtttgaccccagctagagagtgaggggaacaacagcaaaaggcaggacaaagactgactcgtgagaggaggcccgggaacagggggccattgtgaatgaggaggacgtgggggcccaagaaagtgagcaaaagaggacagggcttgcgcactcagtcaccagcccccttctggggtccaagctgtgtccccttctctaaagaggtaagccctgagtcatgggaagatggaaaccggggctcatgagacaggatgttttttaagcaccgtggtgtcttgttgacttgcacatgcacgggggtcttgggtaaccacagggctcagggtatttgcaggaacagttcaagtgctcacttgtcttggggctgtttatggggaagtggtttccacagtgagaggaggtgagatattgttgtcaccccggaccacacttagctacttccttctcactaaagctctgtagtcatattttccctggcagagcagaaacttctatgttatcccacagctgttctaacggtgtagacttgacttatgcaatgatgccaggagtcctgagcagcacagcccaacttcaatcacacacagatggacagagctgtattagcaaagcctgagctactgagcgatgagagtacagccaggctttcagacatctgttcattcaagagagatatgcgctaagccaaggacctaaagatgtgtttaatatgggtgctaatatgcataaggaaccttgaaataaatgttcttagcctttggccaagagggtccatgtctaggaatctattctccatagaaataaattcaaatatggaaaaaatgaacaatgcataagtgtatttggtccccagcatatttatagcaacttaaaattggacccaatttaaatgcctatgatatggaaatggctaagaaaattatgggatcttcccttgattggctattaggcagcctttacaaacaatgcagtgacatgagaaatgcttatgttatggtaagcttaaaaaactcaagatgcaaatcagcttattttaatcaggagccacctagcatttgggatgtggtcaatcccacataatgtatttttgtgggtgcagttcccaggaaagaggaggaataaaaacggcaagtatgaagtgtctccttcgcttgcagtctccttgtctacccctttgtccatccactatgaaaggactcccttctgttccttaatatggacaatttctattgaggactcattgttctaagaattgtctcatctcctcctgcatcctcagtgcccgatctttggcttctatgaaggaaggtgggtagtgcgtatggcaggtccagttctacctttcttagtatgttctggcgtgggtatgtagccccattttctagtggttaccttgacatcatgaagagtttatgtctcttttgccctaggtttgggcaatagtcattcactgtgcaacaggaaatacacgagtcagcatcttattaaaaataaagtcattcaggaaagtggacgacagtttctaatctagagagcataggagaagaaatgtttaccacacacaaagtattagtgccttttatatcacgaagacaaaaataacaggaaaaagacaaacacattatagtgaaaacttgtttttcctaaccagcatctattctgcatgtttcctgatgcccgaaactcacatttcctcaggaaaatctcccttctgcaccattctcaggctttaagtttatgtaaaattcagtaaacccaaagattcaagttatgtgccttgattaacttaagcaaatcaatgaaacccatccccataaccacagcgacaggttaggaaattcggttcctaagtcagtcacatccgaaagggcctagtgatgtttttttccagtgggatcacagactcactcttccttgcagaaaatgaacaaaggattcatgtaacactggcaggtactggcagccacccagggcctctcacaggaaagggagatcagaaagagaagcaaagaggactcatgagataccatagggctgctgcgtccagccttgcctggagctagggccacctcgatgccctatagtcttggagccacaacgtgcatttactcaaagcctctttgagtttggtttgcttgtttgctttctgcctggaaactgccagcatcctgagagatacgagatctgcatctgtgcagagacacagggtttgttaaaagtcacaggccctgactgaagtgtggaactggctgaaatgagaaagtggtaatttggggaggaccttgtgaaatggaaggagttttaaaccttacatgcatcagaattacctggagccttgtgaaaacacaggttgctgggccctagtccattaagaaaggaagtggggcttagaatgttcatttctcccatgttcccaggtgatattcaccatgctgtcctgtctgggcactaccttttgccatacccattacaaggtattgcacgtgctggttgaactatggtctgtcttattttggtgctaaaagcctgtgccaaataccaacgctgcagcattaaggaatgtgatagaaaagattctgaatataggccaggcgcagtggctcacgcctgtaatcccagcactttgggaggccgaagcaggcagatcacgaggtcaggagatcaagaccatcctggctaacatggtgaaaccccgtctctactaaaaatacaaaaaattagccgggcgtagtggtgggcacctgtagtcccagctacttgggaggctgaggcaggagaatggcgtgaacctgggaggcggaacttgcactgggctgagatcgcgctactgcactccactccagcctgggcgacagagcaagacttcgtctcaaaaaa

SEQ ID NO: 62 - DNA, 650 bp, Homo sapiens
ggccatgggggaaaaagtctaactggcggaactcctgggaactggggcgatgggctcttagtatcggaggattggagccatctgatttttacctgaaattccttagtctctcctgtgttggggaaatggtcaccttgccttcagggacctgggctttcagctgtccatacctggccctggttgatggcggcatgctgggcagtgcacgtgaagacgcacatgagacagcatctcgtatgttgcccaggctggccttgaaagcctggcctcaagccatcttcctgcctcagcctcccaagtagctgggatcacagggttgtggcatcacagctggctatattcttaacattattttgtaaccattccaacccccagaaatttctctctggctgacttgatccacagcgcctccatcgccatccctgagtgccttgttgtggaaaatcttactttatcttggttctgtttggtataatcggggaaagtctgtattctttcattatgtaaaacaacttatctctcattgtttcatctcctttctgagctctgctctgccagctctctttccaaaaccaaaatggctcttcaagttattttgtaaataataatgggccatctacttcttaacataaatgaatgattttccaagg

SEQ ID NO: 63 - DNA, 3853 bp, Homo sapiens
cttattgctgggcaggttctcataagaggccatgggaaagccatgtcctatctcagggacacagggtcatctgggcctctggctaatagaggccaaataatgggactattttccctgtgaaatcctgaaaaccaaaaatggtggcgtctttatctgcattagcagaggtaatttgctccttcttgaaatccaaggtcacgtctactgtctggggattttgatccagggtcagtgtggtttctcctttacaggagagccgagtctcagaaaggtgaggtggtttgtgttggtcattggctacctcagattttagagcagctctaccttgattgtggggttgacctaattttttttgctgtcttctttcttctccaggtgaggaaagaggacttcctgtatatctctatccttttgtttccattactcactttctgtggctgctgctgcagaagccactgctgactgatgtggatacctcaatctttggtttacaaaaagcctaggtgtcttttggcctctctccaggttgatagccatggctcctgaaagaaataaaagatgatcatctttctaaaaagtcttaagtctgaattattagtaacttaactggagaatctcacttttcctactctcgtattttaaccacagttgctctaacacagacctttgaggatcttttcatgacttcattcacaaatacctatttatgctgtacagatgctactaggaaggaaatagggatgtctgttttgactgtggaacttaacttggtctcgtctcttcgtgcatgcaaccctgtccttgggatagctttcttgagcatatctacttatgttcaagaggtaaattgtcctgaaacccccattgctataagtatttattttattactcataatacttaatgctcctaaagttggggtattttttttttggatacctaaacttcattgagatactttgaactatttatagagaaaacggaaccttctaatacctggcttctatttcttaaaatgttatgatcatacatggcttagggctttatggccaaataacttcactgaacccaggaaaaagaatagatccatctgaaacagacctgtagcttccagaggcctaaattttcggctccatttgtatccttcattttctgtgaggtaaagaagtggaaggagacaagcctcagcccttcccctggcacctttactcttcgcccttcctcctggcatggtggaaagtgcactggaggaggagtgaagggccctaggtttgcatccatgttctgccacttgccaaccttaatggcccttacaattgatttaccctcatgaaatttggaatgatttctaaagtctttcctcgccctgaatgttaacattttttgatagtcaggactttctgtagcttcaccttccttatttagtgttatttttttctcaagactgaacagagagggaagctgtcaaagtgtgctgggcacacaccctgcagtggggcaatggccaattctaatctcaagtcattaggctgcagtagcatgaccactgcttcctgtctaccctcagagggtagagacagctgagctcctgtagttggggtcaggcccagccactctgtggggacagtgattagtgttgtgtcaccaattcagggaaggagccaccttgtcttattttccctcttgaattatcttgatatgaccccattataaatttccttttgtaaacctctgtctcccaatttctccttttagcttactttctattgaagtagaggaacagagtacaacttccatcctctttcatcagccctgaaagcagaacgcaagcgccgttactgggaactatatccttggctccctggatgtggctattaacttctggcctgccactctatcacatacacatatggagatggtgtcatccatgtaccttaccccgtatttacaacttctatcacccaacagtgccaatggccctgatggtccctctgggagggagagaagagtaagctggagtcaccccttccctgtacttcccacctcgccaggcctgttggtgttagtgtcccttctgatcttggcctgacccctgtgccctgggcactgggctgcaggttggagaggcagcatgatggagtggggataacacatactccaaaaccaaacagaagccagacctgggttgggtcctggcgaaacagtctagaggcttggtgaccttaacctcctaattaatcttcctaagcataagtttccttatcataagttatgtatgataaaattttccttggatgcattcattttagcatgacttgaaattatgtgtgaaggaacctggcccatggaagttgccctgtaaattcagattcactttcccttggacatatggatgacattagctcattacagttatgacctccctaaaactcccaaatattctttaagttcttctcttattttccctttagtttgtagtcatatttcttagttcttatatcagttgggattcccacatcttctagttggacaatattggagaagacaccacattttaactgagttccagtgatatgacaggctttcaattctctaatctcacagaagttagaaaaaaagtagataatcaaaatccacagaaaatatagaagattccattaactctgagaatgattctcaggtatccttaggacctcaagaaagctgttctctcctgggcctgtagagagttcaagtgccaggaatctaccacaaagtagccgggaggtgcagggcagcagggggcacagtgaagtgctgaagggcttctcagtcttctttaattagagtgagaagaaaagagcacctcctcattttagagtacaaggtgtgaactcactctcagctgccaagtgagcttcaccttgggctgttttgcatgctttctcctagtgctttaagccaccctgagatgtacagaccaatactggccatcacaaaaatatactcgagtacatagaccattgacactataaagcaagtaaacaatgaagtctacataacagccaaataacaacatgatgataggatcaaatctgcacatatcaatattaaccttgaatgtaaatgagctaaatgcctcaattaataggcagagagtggcaagttggacagagaagcaagacccaactgtatgtcttcaagagacccatctcatatgcagggacaccaatagcctcaaagtaagggatggagaaagatctatcaagcaaatggaaaacaaaaaacagcactctctgtccaacaaaaacagaatatacattcttttcagctgcacatggtacatactcttaaaatcgaccacaattgctttattggccagaaagcaattctcaacaaattcaagaaacctgaaataccggccaggtgtagtggctcacacctgtaatcccaacactttggaaggctgaggtgggcaaatcacttgaggtcaagagtttgagaccagcctggccaacatggcaaaaacccatctcttctaaaaaatataaaaattagccgtgcatggtggcatgcgcctgtagtcccagctacttcggaggttgagtcacgagaattgcttgaacctgggaggaggaggttgcagtgagctgagatcacgccattgcactccagtctggttgacagagtgagactcatctcaaaaaaacaaaaaaaccctgaaataccaaccacactcttggaccacagtgccataaaaataaataccaagaagatctctcaaaaccatataattaagtggaaattaatctactcctgaatgacttgggtaaacaaagagaaattaaggcagaaatcaagaaattgtttacaact

SEQ ID NO: 64 - DNA, 376 bp, Homo sapiens
ccgtggggctacttccagttcaatgtgaccagcagaaggcacagtactttacaggtcctgcagaatggagggcgtgtaaccagcctggagcaaggaaagagggcgtcctgcagacaggggtgcctgcgctaggttttgaaggataacaggttggccagagcagacaggaacagaagaaccccttctagactatttgaagacaattctccatgggtcgcttgcatttctgcatgtatagtgaaaagtctttgacagcttttattccagactgtctttttaagagtacttgagtatctcagatgatccagatagtttctccctcctggaaagagagcagatttttcctcctgaccaggataataaaatcatacctctc

SEQ ID NO: 65 - DNA, 283 bp, Homo sapiens
ggccgggcatggtggctcacgcctgtaatcccagcactttgggaggccgaggtgggtggatcacttgaggtcaggagttcgagaccagcctggccaacatggtgaaaccccatctctactaaaaatacaaaaattagccaggcgtgagccactgcgcccggccagaatggcattatatttaaatagttcataaagaagcacaaaagaatattatttcataacatgtaaaaattatataaaacgtaaatttccatgttgataaataaagttgtattggaacacg

SEQ ID NO: 66 - DNA, 1574 bp, Homo sapiens
caagaatatagaccttacacataaatagttcagaaaggttgaacaactaaaagatagggactttgataagttatgacatatttttttggaatcaaggagattatgtacatgcataaagctgtgtgcatactcaggaaaaagctgagaaggccctaaactctcaccaatggctgaccttgaggcactgcataagtaggtgaaggctaaggagaagctgttaacttgtggctaagtattaaaggtgtgccccaacacacagagtccccaatacaaagagaagtattgattccaggcatttaaggaaatctgtccaattattagcacactactaagcatatgaatcagatatttcatacacaacaaagaatatagactttacaaatatatagttcagaaaggtcagtaaacagcaaaatatagcaacaacagcaaaacctggtgaggaaagggagtctgatatacagagttgtaacatgttatttaaaatgtccaattttcaccaaaaaattatgagacatgcacaaaaacaagcaagaatggtccatgcactgatggggaaaaaagcaatagaaattccctgaggaagcccagacttcacacttactcaaaaaagacattgaaaacgctattttaaatatgttcaaagaaccaaagtaaacaacgtctcaccaaatagagaaaatcaataatgagatagaaattacgaagaaaagccaaaaaggaatgaacagattctcagagacctgtgggacactgtcagatgtaccaacataggcatgatgaaagtctcatgtcaaccataattttcacccatagccatacatgcccaagagaattgaaaacatgtaatacttgaatgtgaatgttcatagtggcataatagctaaaaaaaaagaacccagatatccatcatctgatgatgagtgaacagtgtggtttatgcatacagtggactggattcaggcataaaaaggaatgaagtattgatacagactacaacgtgaatggatgagccttaagaatatcatgctaacaaagaagctaaacacacaatatggttccacttacatgcaatgtccaaaataagtaaatccatagagactgaaaatacatcagtgattgctaggagctgggagagggaagaatagtgagtgcatgctaatgagtctgacatttacttttagaaagatgaatgtattctggaattggatagcgctgattatacgaccttgtgaatatacaggatccactgaactgcactttaaaagggtgaatattgtgtgaatcatatctcaatttaaaaagatatatataaagttccctgggtgaatactggtttccctcctcccttcagtatatgtgaaatgtagtgaaatttatatggttctgacagtattttattttaatgatttttcctccatccttggtagttttttttttttcctttatgtatatgaaacggcaacactgttcgtgaagtcagagctacacaaaatactataatcggaggagtggcaactctcccctttcccattgttgttctttccaccccattcccgcccgccccctgtaaa

SEQ ID NO: 67 - DNA, 430 bp, Homo sapiens
agcctcccgagtagtcgggattacaggcgcccaccactaggcccagccaatttttgtatttttactagatacggggtttcacgatgttggccaggctggtctcaaactcctgaccttgtgattcacctgtctcggcctcccgcctcagtcccccaagtagctgggactacaagcgcgggtcaccacacccagctaatttttgtatttttagtagagatggggtttcacaatgttgaccaggctggtctcaaactcctgacctcaggcagtcctcctgcctggcctcccaaagtcctggggttacaggcatgagccattgggcctggcctaccctgattcttaagaaagcattttctttctttcatattataaagtagttatgtgtaggtttatttagttaggaattccagctgttcagagatggcaaaac

SEQ ID NO: 68 - DNA, 677 bp, Homo sapiens
acagttcatcatattgcttcatatttctagattcctaggaaatgtattctagattcatttctgggagctaagcaggaactgtgtataccagttgaattcagcccatgctgattgtgcacctgtggttaaataaggtgcagcaggcaagagagagaagtatgtttagacagcatctgccctcaaggagtttgtaacctagttgagaatcttgaaatctgttttctagtttgctagtttctagttggctttaactaattaattaattttagattcaggatactactgtgtaagtaaaacttaattacatttgacatgatacagttggccctccatatctgcggtttccacatctgtggattcaaccaaacttggattgaaaatattcagcaaaaggccaggcactgtggctcatgcctgtaatcccagcactttgggaggctgaggcaggcgaatcacgaggtcaggagatcaagaccatcctggctaatacggtgaaactccgtctctactaaaaatacaaaaaattagccgggcgtggtggtgagcacctatagtctcagctactcgggaggctgaggcaggagaatggcgtgaacctgggaggcagagcttgcagtgaaccgagatcacgccactgcactccatccagcctaggcaacagagtgagactctgtctc

SEQ ID NO: 69 - DNA, 554 bp, Homo sapiens
cgggtttgaactgcgagagtccacttatatgagggtttttgaaaataaaagttaccccgagtgtgcctgcctctcctgccttcccttccacgtcctccacctcttctgcctctgccacccctgagacagcaagaccaacccccggcttctcctcctcagtctactcaacctgacgatcacaaggatgaagacctttatgactcacctttatgattcacttccaacatatgactttgtacagaaatcaggaagatgctttgaaagaaacactgtgcaatgaaagtgccactgatgtgtctagcattgacatgcttttggctgcaaagacttgagcccacacgttgcctgaaaccttcagtcctttgaagctgtgatttcaagacccagatgttgacagctgctcagaatgcttctcaggaagaggctgggacttccaagaccccattcctgggttgggtgatgagtggttctgatactgtgaaaactcacaaaagactatgtaatgataccaaccacgtgagactattttgagaattaaatgagttaatatatgc

SEQ ID NO: 70 - DNA, 1702 bp, Homo sapiens
gcggccgcaagggcttggctgggccgcgggaggcgggaggttcttcgtcctcccgagccatctccctgaactgacaagcaggactcccgggtccagggggcacagggcccggggcggtgaccctgcggatcgggctgccggaggagcccactgtaaatgccgcaactggccccaaacactgcgttcctggactgcaccagcagctcctggcgcggccgcagagttggtggatattttccaagggggaaaaaaatcttttaaatgccatctgtttactttaaaaatgttgattacttaagaaaaacgaatggatgtctgggcaaaggtatggacgtcacaattattttgaaggcgtcctttttaactttaaacagaccacgccaggaggagactgctgacccagagcgcattacctaaaatctggtacccagagtgcacccttcgccctcgttggagttctctcctctctgccaagctttgctccgtgccagaggtgtgctccattgtacctccgctctgtccctgcagtcaggcaaccaattggagaagagtataaatagtaattaaccagggagagttgtaattcagaaacctagttaaaacaagtcctcaaaaactagagaatatgagagtggggagacattttgaaggcattaagaacaaaaaacgatggggacgaatggttgagtctgaggatcagcatcgtaatctgttagagaacgaggtcgtggctgtgtctgtgagtcgttaatgggtttaatcggttgatacacagcctgctagtggcctaaccagtaacccagggcctggcagatttgcatgacatctcggagtttgattgctcttccttccacttggcaaaaggagacaccatcagccggatcaggaggggtcatggtgagatggaacccaccgaggtggtgtacagagctggcgctgccaatggccagagtggcagcctttctacctccttaaccctgcaaaaatcaaacgtgctagtacgcactgtccatccacactggaactccagttggttttagtctgcgatgatgactcttctgggttgacttttccagttcacagcctttctacctccttaaccctgcaaaaatcaaacgtgctagtacgcactgtccatccacactggaactccagttggttttagtctgcgatgatgactcttctgggttgacttttccagttcattatgcagccctcttgaagcaggcctcccaaacttagcagacaccaatgagaacctcacaaagaggctcatcaagcaggctggtgaaactgggtgttacttcctgttccatgggtaccccatagtgtttgggaaacaccgggctgtggttcaggagaatttcacatatgctaagatggagaaagaacctgccctttacatttaggcttgggatgttaatttaaagtttgaatgaccaaaaattaaatctgtaacttttaaagtttctctttgtgattttacttaagtgttggtagatattcttaaattgtaatgacctcagtttgggaattaagttagccaaatattgtgtaattattgtttgttatacaaaaatatgccttagactgtacagcggcagaaactccctctaccacctcggtccccctttccattctgcgttatacaaaataagctgacacgttaatgctgtggcccacattaaacaaagtataccgt

SEQ ID NO: 71 - DNA, 567 bp, Homo sapiens
cactgcgcccagcaggaatattcctaaatataagaggtgtgtctgccacccgcccttctcaagtggagctctgggttgagagagggagggggtgaattttgggctaaggagcctgctgatgtcacttttcttgtcttttcaattatctgtattggctttttgattgtcaaagtaaaaaaatgtgaagattacaggaatcatgtcctgataatagctacctcatatcaagccctcactatgtgccaggcaccttctggggacttggctgcagttgtctgttactcttcacacaagctcaatgaggcggtcctgttattaccatttttattttaagaatgaggagaatgcagcttcaagaaggtaagcaacttgccgaccgtcacacagcttagccgaggaagagccaggcttcacacacgggccttgccgcctctagactacgtgtttattttttagactgagcacttttaaaagagtggcttattttttttgttttgaatttaaaggtcacaaagacacacagaaattgtttgctatctctcttccaagataacctctgttgatatg

SEQ ID NO: 72 - DNA, 1465 bp, Homo sapiens
gttcccaggctggggcgattgccgtcacccctgaacttccccgttcctcttctcggctgcctccttttccgttgtcccttcgcgccccaaaccacatcctggagcgcactctccagcgtggctggcagcggggacggtgcgccggggcgcaggcccaagagtcgcgtgcgcggccccttgcaccatccccccgggcccacccccgggccgcgctgattgggcaggtagggactctgcccagcggaaagttttgggtgccgggaggaagtctaacctttgggagactccaagacagcagctccgaggtcggcgggggtctgggtggccatggaggagccccctgtgcgagaagaggaagaggaggagggagaggaggacgaggagagggacgaggttgggcccgagggggcgctgggcaagagccccttccagctgaccgccgaggacgtgtatgacatctcctacctgttgggccgcgagcttatggccctgggcagcgacccccgggtgacgcagctgcagttcaaagtcgtccgcgtcctggagatgctggaggcgctggtgaatgagggcagcctggcgctggaggagctgaagatggagagggaccacctcaggaaggaggtggaggggctgcggagacagagccctccggccagcggggaggtgaacctgggcccaaacaaaatggtggttgacctgacagatcccaaccgaccccgcttcactctgcaggagctaagggatgtgctgcaggaacgcaacaaactcaagtcgcagctcctggtggtgcaggaagagctgcagtgctacaagagtggcctgattccaccaagagaaggcccaggaggaagaagagaaaaagatgctgtggttactagtgccaaaaatgctggcaggaacaaggaggagaagacaatcataaaaaagctgttcttttttcgatcggggaaacagacctagatccaaggccacaagtaaggctatggctctgattctagaagacaaccttccaagatgcctggcaaaaccacctccctgtgccacacagacacactaggcctgtgtatttatttccccttcaaagcagactgaggagggaggagacgaggttctcttggcatcactttctccctggctgcagaactagacacccttgaagatttggcctgggccagtgagactgaaatcaagaaaaacagaagggatgtgcagggtgggggggtccacttcctgctcccatgtcaacccccagggcctccagcgtgcagacgcgtgtcctactcatctgctcccacggatgaccctggtcttcaatggttagcagaagggagaaaagaaagcaggaaaatgtgctattgagattccagtggtgacttcactgatatttagtgaatatttgatttagccaacatgcctttctttatgtgattttgtattaaagtaaaatgatttttatactttc

SEQ ID NO: 73 - DNA, 965 bp, Homo sapiens
gatcaagttctagagtggaactatcacaggggctgtgaggacttgggagaagagatcatatggtcacttgttttttggaagagatgaagaaaggcatgaaatagcctgttaaaagtgaaaaggttaacgaagtttctcagggcaaagatgagaatccagctctgtttcaatagtgtttagttgaggcaaccaggagatatactaacagtgatcctgcctcaaggaaaagataaacacttctgggagtccattttataacccagtctgcccctgataccatagaaaactagtaaaagcagctgtgggtccccaaacttctatggaacagctttggatatggcatttttagtttttaataacagggaaaaagtagaggaagcaaaaagagcaagaaggacctcccacaaggtgcagctcttggttgcaaccttaagctaacctcccacatggggctgcctcctgagtcttggcctgaacaatagaaactgaaaggtgggaagaccaaagctggccatctgagtcactgtgccttgggcataaatcagggtgcacactgtaagaaaactggccattggaagagggataatccagtgttctgaagagagccatcggcaccctaactaatgatgagttaaacagccaggcaagtgcccaaaagtgatggggcccgagaccttccaccaaagctccaatcagacaactagccatattatctggagaagccttggtaaccttcaccatggcaggtaagaatattaactttcaccaggcatggtgactcacacctataattctagtatattgggaggccaaggtgggtggataacttgaggtcaggagttcaagactagcctggccaacatggtgaattcccatctctaataaaaatgcaaaaaaaaaaaaagccagaaactgcttgaacccgagaggtagaggttgcagtaagctgagattgtgccactgca

SEQ ID NO: 74 - DNA, 1807 bp, Homo sapiens
atggacggcaacgacaacgtgaccctgctcttcgcccctctgctgcgggacaactacaccctggcgcccaatgccagcagcctgggccccggcacgaacctcgccctcgcccctgcctccagcgccggccccgccctgggctcagcctcgggccggtaccgagcttcggcttcagcccggccccactccgaccccggagcccacgaccagcggcctcgcgggcggcgcggcgagccacggcccttccccgttccctcggccctgggcgccccacgcgctcccgttctgggacacgccgctgaaccacgggctgaacgtgttcgtgggcgccgcctgtgcatcaccatgctgggcctgggctgcacggtggacgtgaaccacttcggggcgcacgtccgtcggcccgtggcggcgctgctggcagctctgccagttcggcctcctgccgctgctggccttcctgctggccctcgccttcaagctggacgaggtggccgccgtgggctgctcctgtgtggctgctgtcccggcggcaatctctccaatcttatgtccctgctggttgacggcgacatgaacctcagacgtgctgctctcttggcactctcctcggatgtaggttctgcccagacttcaaccccgggacttgcagtctccccgttccacctctactcaacatacaagaaaaaggttagctggctgtttgactcaaagctcgttctgatttctgcacattcccttttctgcagcatcatcatgaccatctcctccacgcttctggccctcgtcttgatgcccctgtgcctgtggatctacagctgggcttggatcaacacccctatcgtgcagttactacccctagggaccgtgaccctgactctctgcagcactctcatacctatcgggttgggcgtcttcattcgctacaaatacagccgggtggctgactacattgtgaaggtttccctgtggtctctgctagtgactctggtggtccttttcataatgaccggcactatgttaggacctgaactgctggcaagtatccctgcagctgtttatgtgatagcaatttttatgcctttggcagcgtacgcttcaggttatggtttagctactctcttccatcttccacccaactgcaagaggactgtatgtctggaaacaggtagtcagaatgtgcagctctgtacagccattctaaaactggcctttccaccgcaattcataggaagcatgtacatgtttcctttgctgtatgcacttttccagtctgcagaagcggggatttttgttttaatctataaaatgtatggaagtgaaatgttgcacaagcgagatcctctagatgaagatgaagatacagatatttcttataaaaaactaaaagaagaggaaatggcagacacttcctatggcacagtgaaagcagaaaatataataatgatggaaaccgctcagacttctctctaaatgtaataatgatggaaaccgctcagacttctctctaaatgtggagatacacaggagcttctatcttgctgaaatattgcttcatatttatagcctgtggtagtgcacatggttaacataaaagataacactggttcacatcatacatgtaacaattctgatctttttaaggttcactggtgtattaaccaaacgttgtcacaaattacaaatcaatgctgtaatataatttgcacctggaatggctaacgtgaagcctgaattaaatgtggtttttagtttttaccatcaccaatttctatgactgttgcaaatacagaatctattagaaaac

SEQ ID NO: 75 - DNA, 535 bp, Homo sapiens
gagcagattcgcacaaacccggaagcgggtcgcgtggagtgacggtcccaccgcggggatatctcttccaaatgcatgatgaaggagttctcatccacagcgcaaggcaatacagaagtgatccacacagggacattgcaaagacatgaaagtcatcacattagagatttttgcttccaggaaattgagaaagatattcataactttgagtttcagtggcaagaagaggaaaggaatggtcacgaagcacccatgacagaaatcaaagagttgactggtagtacagaccgacatgatcaaaggcatgctggaaacaagcctattaaagatcagcttggatccagctttcattcgcatctgcctgaactccacatatttcagcctgaatggaaaattggtaatcaagttgagaagtctatcatcaatgcctccttaattttgacatcccaaagaatttcttgtagtcccaaaacccgtatttctaataactatgggaataattccctccattcttcattacccatacaaaaattgg

SEQ ID NO: 76 - DNA, 2450 bp, Homo sapiens
ctttccagccgcggccgacgcaccccggccgccgccatgagcggctcctcaggcaccccgtatctgggcagcaagatcagcctcatctccaaggcgcagatccgctacgagggcattctctacaccatcgacaccgacaactccaccgtggcgctcgccaaagtgaggtcctttggcactgaagaccgtcccacagataggcctgcgccccccagagaggagatttatgagtacatcattttccgaggaagtgacatcaaggatatcactgtgtgtgaacctccgaaagctcagcacacactcccgcaggatcccgccattgttcagtcttccctgggttctgcctccgcctcgcccttccagccgcacgtgccttacagccctttccgagggatggcgccctacggcccgctggcggccagctccctgctcagccagcagtatgccgcctccctgggtctaggagctggttttccatccatcccagtcggcaagagccccatggtggagcaggctgtgcagactggttctgctgacaacctgaatgctaaaaagctgttacctggcaagggcaccacagggacgcagctcaacggtcgtcaggcccagccgagcagcaagacggccagcgatgtagtccagccggcagctgtgcaagctcaagggcaggtgaatgacgagaacagaagacctcagaggaggcgatcaggaaacaggcgaacaaggaatcgctccagagggcaaaaccgtccaactaacgttaaggaaaacacaatcaaatttgagggtgactttgatttcgagagtgcaaatgcccagttcaaccgagaggagcttgacaaagaatttaagaagaaactgaattttaaagatgacaaggctgagaagggggaagagaaggacctggctgtggtgacccagagtgccgaagcgcccgctgaggaagaccttctggggcccaactgctactatgacaaatccaagtcgttcttcgacaacatctcttctgaactcaagaccagctccaggcggacgacgtgggccgaagagaggaagctcaacacagagacctttggggtgtcagggaggtttcttcgtggccgcagttctcggggcggattccgaggaggcaggggcaatgggaccacccgtcgcaaccccacttcccacagggccgggactggcagggtgtgagggtgcagccaaaggctcctactgaagtggcgcataactgacgctgtgtgtgtcaggacgcgaggaaaacgctgcacttacagggagaggtggtcactttgtttacggagtttggaagagacccatactgctacttgtgttttggacttaactgaacttggacatggtctgagttagaaccacttgttttggggaagtattcatgggtaacctctttgaggtctctttatctgtgtttcctttttagttgcgcatagcctaattctaaggttttggtattttgcaaaaaggtttctatagtgaaagctgaatccttactttgtgacttttttttttttttttaatgacaagctttgacttttaaaagtggaaccaaatctgttggcagaggtggcagccaagtacatctctgtaacccagctggcccctggtgctgttggcctggcaccccactgccaagggtggggtctcaggagtcaggcagggccagcacagggtggcgtggggggcaggggtgggtgggtggagggcacggaaggggttttcccatggatcatgttgtataagtgaaccagaccaccctgatggcatccacagtgatgtcaaggttggggctggccaggggtgggtggactagaagcatttgggagtagtggccaggggccctggacgctagccacggagctgctgcacagagcctggtgtccacaagcttccaggttggggttggagcctgggatgagccccggcagcgccttggcccttctgtggtccctgccagcctctgacctgggccggtcagtcattgctggactctggccacacactggcgttctcatccacttggaaacaagccagtcttttctgcaaggtcagttgaccaagagcatatttcccctctgttgtacatcgttgttttgtgtttgtgttgtaacagtgggtggagggagggtggggtctacatttgttgcatgagtcgatgggtcagaactttagtatacgcatgcgtcctctgagtgacagggcattttgtcgaaaataagcaccttggtaactaaacccctctaatagctataaaggctttagttctgtattgattaagttactgtaaaagcttgggtttatttttgtaggacttaatggctaagaattagaacatagcaagggggctcctctgttggagtaatgtaaattgtaattataaataaacatgcaaacctttaaaattttcttttctgatgctctaagaatcctgt

SEQ ID NO: 77 - DNA, 2395 bp, Homo sapiens
gggcggttgtgacgttgctagcgcttgtccggtggctgctgcgctgccgcaacgaatagggtttctggctgcgtaggagggacgggggcgcggagctctgggaaactgcgccaggcgcccgaaaggtgaacacgggagtcgcgcgtctcccccgcagcagcggtaaagcggaagttatgctgcagccggagcccgggcttcctcccggagccgcgtcccggggcccggctgccccgagctgagcggagcatcctttccgggtgaggggaggagaggacttggcgcgttcccctcgctgccccgggagccgcagccgcggtgttcatgccgcggagcagccaggctcctccgacgaaaacctgcatttatttgctggcgggacgtttgccttgaaaatggacaaagacgccgccctccggggtattcctgtttgcctgaccctgagagcgcctttttgcttcaagacgtgttggatgctcctgttctccgaattctgatacgcttctgggcataatactgaaacacaaaactgcttttgctctctctgtggttggccgaaaataggattctttttcgtgcaggtgtcgttgtttagtcggctttactaacatattgaaatggctctacccaaagacgccatcccctcgctgtccgagtgccagtgcgggatctgcatggaaatcctcgtggagcccgtcaccctcccgtgtaaccacacgctgtgtaaaccgtgcttccagtcgaccgtcgaaaaggcgagtttatgctgtcccttctgtcgccgccgggtatcgtcgtggactcggtaccatacccgaagaaattctctcgtcaacgtggaactgtggacgataattcaaaaacactatcccagggagtgcaagcttagagcgtctggccaagaatcagaggaagtgggtgatgactatcagccagttcgtctgctcagtaaacctggggaactgagaagagaatatgaagaggaaataagcaaggtggcggcagagcgacgggccagcgaggaagaagaaaacaaagccagtgaagaatacatacagaggttgttggcagaggaggaagaagaggaaaaaagacaggcagaaaaaaggcgaagagcgatggaagaacaactgaaaagtgatgaggaactggcaagaaagctaagcattaacaatttctgtgagggaagtatctcggcttctcccttgaattccagaaaatctgatccagttacacccaagtctgaaaagaaaagtaagaacaaacaaagaaacactggagatattcagaagtatttgacaccgaaatctcagtttgggtcagcctcacactctgaagctgtacaagaagtcaggaaagactccgtatctaaggacattgacagtagtgataggaaaagcccaacagggcaagacacagaaatagaagatatgccgacactttctccacagatatcccttggagttggagaacaaggtgcagattcttcaatagagtcccctatgccatggttatgtgcctgtggtgccgaatggtaccatgaaggaaacgtcaaaacaagaccaagcaatcatgggaaagagttatgtgtcttaagtcacgagcgacctaaaaccagagttccctactcgaaagaaactgcagttatgccttgtggcagaacagaaagtgggtgcgcccccacatcaggggtgacacagacaaatggaaacaacacaggtgagacagaaaatgaagagtcgtgcctactgatcagtaaggagatttccaaaagaaaaaaccaagaatcttcctttgaagcagtcaaggatcaatgcttttctgcaaaaagaagaaaagtgtcccccgaatcttccccagatcaagaggaaacagaaataaactttacccaaaaactgatagatttggagcatctactgtttgagagacataaacaagaagaacaggacaggttattggcattacaacttcagaaggaggtggataaagagcaaatggtgccaaaccggcaaaaaggatccccagatgagtatcacttacgcgctacatcctcccctccagacaaagtgctaaatggacagaggaagaatcccaaagatgggaacttcaaaaggcaaactcacacaaagcatccaacaccagagagaggctcaagggacaaaaataggcaagtgtctttaaagatgcagttgaagcagtcagttaatagaagaaagatgccaaattctactagagatcactgtaaggtatccaaaagtgctcactccctacagcctagcatttcacagaaaagtgtttttcagatgtttcagagatgcacaaagtaaggcctggtaaagggagtgctttgtgatctagtaaagctggaatgtgaagctctttcctaaaaaaaaaa

SEQ ID NO: 78 - DNA, 5075 bp, Homo sapiens
ccgtgacctccatgtgggagctccagctctataagtaaacactctgcgcggcgcagacatggcctcttcctatctttgaggcggtgtctgcggcagcgcctcagagtggttccggtcgtctctcctcaagtcggctagtcgggcgcgcgcgctgagagtcgtcgccgcctgtcgggcccggcgtccggtcggtccggtgggcgcgctcgcccgcctgccgctgagggcccgagccgcagggaaagcggcgcgggccgggcggggcgcggcgcccagagctcagggggagacaaaggggaccggttcctctctaggcgccaagatgtggatacaggttcgcaccattgatggctccaagacgtgcaccattgaggacgtgtctcgcaaagccacgattgaggagctgcgcgagcgggtgtgggcgctgttcgacgtgcggcccgaatgccagcgcctcttctaccggggcaagcagttggaaaatggatataccttatttgattatgatgttggactgaatgatataattcagctgctagttcgcccagaccctgatcatcttcctggcacatctacacagattgaggctaaaccctgttctaatagtccacctaaagtaaagaaagctccgagggtaggaccttccaatcagccatctacatcagctcgtgcccgtcttattgatcctggctttggaatatataaggtaaatgaattggtggatgccagagatgtcggccttggtgcttggtttgaagcacacatacatagtgttactagagcttctgatggacagtcacgtggcaaaactccactgaagaatggcagttcttgtaaaaggactaatggaaatataaagcataaatccaaagagaacacaaataaattggacagtgtaccctctacgtctaattcagactgtgttgctgctgatgaagacgttatttaccatatccagtatgatgaatacccagaaagcggtactctagaaatgaatgtcaaggatcttagaccacgagctagaaccattttgaaatggaatgaactaaatgttggtgatgtggtaatggttaattataatgtagaaagtcctggacaaagaggattctggtttgatgcagaaattaccacattgaagacaatctcaaggaccaaaaaagaacttcgtgtgaaaattttcctggggggttctgaaggaacattaaatgactgcaagataatatctgtagatgaaatcttcaagattgagagacctggagcccatcccctttcatttgcagatggaaagtttttaaggcgaaatgaccctgaatgtgacctgtgtggtggagacccagaaaagaaatgtcattcttgctcctgtcgtgtatgtggtgggaaacatgaacccaacatgcagcttctgtgtgatgaatgtaatgtggcttatcatatttactgtctgaatccacctttggataaagtcccagaagaggaatactggtattgtccttcttgtaaaactgattccagtgaagttgtaaaggctggtgaaagactcaagatgagtaaaaagaaagcaaagatgccgtcagctagtactgaaagccgaagagactggggcaggggaatggcttgtgttggtcgtacgagagaatgtactattgtcccttctaatcattatggacccattcctggtattcctgttggatcaacttggagatttagagttcaggtgagcgaagcaggtgttcacagaccccatgttggtggaattcatggtcgaagtaatgatggggcttattctcttgtactggctggtggatttgcggatgaagtcgaccgaggtgatgagttcacatacactggaagcggtggtaaaaatcttgctggtaacaaaagaattggtgcaccttcagctgatcaaacattaacaaacatgaacagggcattggccctaaactgtgatgctccattggatgataaaattggagcagagtctcggaattggagagctggtaagccagtcagagtgatacgcagttttaaagggaggaagatcagcaaatatgctcctgaagaaggcaacagatatgatggcatttataaggtggtgaaatactggccagagatttcatcaagccatggattcttggtttggcgctatcttttaagaagagatgatgttgaacctgctccttggacctctgaaggaatagaacggtcaaggagattatgtctacgtgggttgtgcttgggaaaagttggacctgttaattaaaagtaaaatatttccaaatcaatttggaaatgacttgaagtgtgagggaaagggattcataaaatttaggtataggaggccctggaaaaggacatttatcctagagggcacagggggtgtctctctggtaggggaagggtggggaggtggctttataagagtggtctgccttctccctttctcacttttcctcaccccttttctctcttcccccgcaaagctgcttccctgccctgccaccacctttagtgctttgtcttttttcccctttgcccatgctcagctgttaacccataaagacttcgttgattttgtgtgcatagtggatggtatggctgcattaatcccttcactgcctgtataccctagaatttgtccctgacactgacttcagagcatggtttgagttcatctcccatcattccccattgttgtgcttcccgtaaaaactgccagctttatcatttcccctggctctgcccacactgcatgtgtaggggctgaactatgggcaagtgtctgaccacccaggcaggtgagtgtgtgtcttctaatgcaagtctgtttctgtttttgttgtctttttaaactcatagaattgattgttgaaaataaggccatcaactgctaaaacaactactaaaataattctttttaatataaaaataactttgtcaaattcactttcagaagatttttcagatgtccctgttgagagcattgttctagataggttatatttgaaactgtgagcagaagcatgtgagcccatctgctatgatgagtaatagtcattgaggcctgaaacatacagtgctttaagcatgactgttattacaaagcatgcttctcccaccccacccaccccctcaaagaaggtagccattgaaacataaggatgatagatagaatgtattacttcaaatctaactcttagctggtggaggatttagtaatttagttgctttaggtcttgtaaaagctcctgccgctaactttaggagatgagaagtttgacccttaatgttcttgatatttttttagatcaactccacaatttactgtgatccaatccatctgctttctatctgttgtgctctatgattggttctcatttaccttcatttctgtattctactttccttaaactttaaggaaatctaatcacaactcctgaagacttacctttcttagatctgaaacttaagatcagtgtattataaaatggaatctcttagcagtcacagctacataaattgggattttaatagttgtctgtgctttgaattcttttcctttaaatgtctgtttcttttatgtaaagtttttcagtttggggaacgtgtagtcttcccctcccttttaatttctcaccaggatctaaaccccccttctctgtgaagcttaaatctgcattgtactctccctcctccccccccatcagtatccagcaggttacccttcagataaagaagggaagaagcctaaaggacagtcaaagaagcagcccagtggaaccacaaaaaggccaatttcagatgatgactgtccaagtgcctccaaagtgtacaaagcatcagattcagcagaagcaattgaggcttttcaactaactcctcaacagcaacatctcatcagagaagattgtcaaaaccagaagctgtgggatgaagtgctttcacatcttgtggaaggaccaaattttctgaaaaaattggaacaatcttttatgtgcgtttgctgtcaggagctagtttaccagcctgtgacaactgagtgcttccacaatgtctgtaaagattgcctacagcgctcctttaaggcacaggttttctcctgccctgcttgccggcatgatcttggccagaattacatcatgattcccaatgagattctgcagactctacttgaccttttcttccctggctacagcaaaggacgatgatctgcctgctttcactgtgttgttcatggtggctttttggacaataaagaatctaaaatgggtggggagggtggaagaaatggtggactgtatctctcacgttctgaagcagctaatcctctttcccacatagccatcatcttgtgtgtgtagtaagaggcccatttctcaactgtcttttaaatatctaaaggtagttcctgtaacaactagttttaatgagtaaaaagtcaaagcctcagctctagttgatatccaagttatgatttattttgcaactacctcaggacagaaaagatttatggggattttaaaaatcattgaataactagttaaatgaaattttagctacacactgcctcccaaatattagttgtgcctggttcttgtaatttgattttacagaaaaggaaatgacacttgagatccttggaatgaacacagcttctaaagtgtgcatatacttttttaacgtctcttcttccattacaatgtgtgttttgcaaggacaggttcattttttttagcccactttgtgaactccattgtgcttttttctggtgttttatgcaagttgactactaatgactaatgagaacaataatgaatgcattgttgctgcattagtgtaatgtggtgtggttttgcacttaaaataggtattcatatgctctacttgtcaatgttcatgaaaatccacttctctactagtcgaactgctttccccctctcaccagtggttttacataagcaaaaaaatgagggctgtgctgacctttgagaggatttgaaattgcttcatattgtgatcctaaattttatattcactatattccctaaagtataccttaataaatattttatgatcag

SEQ ID NO: 79 - DNA, 2259 bp, Homo sapiens
gaggtcgcgtagggcctattatgatgatttctacaggaggttgaagagataagacccttccctgtgctcccccccccccactccttaattacggattgagcaggggaggggccggtggggctcaggtgagcacacagggagaaagggacgtgggcggggccttacagagggtgagcgaatccgaaaagacctagaacctcgttgctgggagacaagtcccgccctgcaatgattaaatcatcatcattaaccagggcctgccccccccatccccggcagcaggggggagaatgggggaataagatcactaccaagtccctgggggtctctcactccccatcccccggcaccctctccgagactctgcaaagcccaagaaactccctccgtgaagccgggagaagacccgccatctggacgaagctccgctacgcggacgccgacagggcggcattacgaggagaggacccaggaggggcttcttcagcagggtcgtcgtcacagaagaccgacgaccctgagcgggtagcgggcacagactgccaggcctttgggggcggcaccggaagtggccggctgggatcagcctttaagatggcgtctcctcaggggggccagattgcgatcgcgatgaggcttcggaaccagctccagtcagtgtacaagatggacccgctacggaacgaggtgcaagggcggcagggttactgctgtggtcggccagcggaggaggttcgagtgaagatcaaagacttgaatgaacacattgtttgctgcctatgcgccggctacttcgtggatgccaccaccatcacagagtgtcttcatactttctgcaagagttgtattgtgaagtacctccaaactagcaagtactgccccatgtgcaacattaagatccacgagacacagccactgctcaacctcaaactggaccgggtcatgcaggacatcgtgtataagctggtgcctggcttgcaagacagtgaagagaaacggattcgggaattctaccagtcccgaggtttggaccgggtcacccagcccactggggaaggtatgtccttggccgcgggacagtaaagaccccagagcattcttcttgcccagttttgctctctggggaaagaggagtatggaatgtgtgccaccagccacctcactaccctatctttctcagagccagcactgagcaacctcggcctccccttcagcagctttgaccactctaaagcccactactatcgctatgatgagcagttgaacctgtgcctggagcggctgaggtgaggagaaggtcaggggttgcaggaggtgacagtgccaatgacccagagccagggagggtctaggggagaggctgagcagtgagtgagtgcctatccccttgaagagagtatatcatggctctgggtggggaagaggaggaaagataggattccctaacctgtgtctatttccccccagttctggcaaagacaagaataaaagcgtcctgcaggtgagaagggctgaggggagggcctctctaaggagactcacctcccatggtccttccctcacacaccttgccctcttccctcccctccctgctcccagaacaagtatgtccgatgttctgttagagctgaggtacgccatctccggagggtcctgtgtcaccgcttgatgctaaaccctcagcatgtgcagctcctttttgacaatgaagttctccctgatcacatgacaatgaagcagatatggctctcccgctggttcggcaaggactcacatccaaaggcgacagcaccaggatttgctcccgcctttggcacagaggaggacgggtccctctctcagcctggccagtctttcccagggcttgatgggaaaaaggacttccctagaaggggttattccgagggtcctccaaccctgctacacattcacagaattcagtggaatgtccgggccggcaatccgagactaaaggtcgtttattgataagccaggccaccctccctgggatcacacccccttcagactccccccaaccatcctacagtcctcaggggaagggtgggctgaggggccctttgaataatataagaacattccccactgactactacttcctcattctctccttagccatcccctttgcttttacaatacagtgtgaaagagaagaggaggtaggggccaagcccccaccccatcccactccccttccctccccagatatttatgtgaaatgaactgcagctttattttttg

SEQ ID NO: 80 - DNA, 1519 bp, Homo sapiens
cctccttgctttcaggactcagtttcctgggttccccttcacggcccctcatctccttacagtccagggtctgagggtctccgcggtcccctccctactcagtcacgccattcttttgaaacgtacacgtgaccgcggcacttcttaaggagcgccccccttttcctcggtggctttcagtttcctcacctcccgcggagaccacggccatggtcatttatccacttgacaaacatttcacgagcccctgccggtccaagctgtggggacgccgtactcccgggcctatggtgcagcaggggaggcaggcgcgtcaccgggaggtcccgagacactaggatccctgccaggccagaggcgaccaaccgtcctggatacgggagctcccggccagcctgacttccaggaggaagcggtgtggggattacctccgaccgcctttagtgccccctgagacctggttctggcctctacgtttcagcccgctactggctcgcacgacccagcgccgccgtggtcccttctcagcgccttctgctccagcgaccatcatgttcccgggtccgagcagccagggccgcggtcaccgcttctctcgcacctcaggccgagaacccacaacgcggcgtgtccctcgcgcgactccgtcgccacgccacgcccccttcccgttctccggaagtgcgcgggttggagcggaagcgcacgagcaaaatgttagtttctcattgtgagtgattcaagaaaacaacggtaacagccctgctaggatcagcggtggtggttccgcgatggtaggcggcggcggggtcggcggcggcctcctggagaatgccaaccccctcatctaccagcgctctggggagcggcctgtgacggcaggcgaggaggacgagcaggttcccgacagcatcgacgcacgcgagatcttcgatctgattcgctccatcaatgacccggagcatccactgacgctagaggagttgaacgtagtagagcaggtgcgggttcaggtgagtcacttccgaggggagcgagttgttccagagagtcagaaaggtttctgtgcagcaggagctggcgtgctctatgctcacgaacaccgaagggttagcgaccccgagagtacagtggctgtggctttcacaccaaccattccgcactgcagcatggccacccttattggtctgtccatcaaggtcaagcttctgcgctcccttcctcagcgtttcaagatggacgtgcacattactccggggacccatgcctcagagcatgcagtgaacaagcaacttgcagataaggagcgggtggcagctgccctggagaacacccacctcttggaggttgtgaatcagtgcctgtcagcccgctcctgagcctggcctttgacccctcagcctgcatactggtatcctggtcccagctcctgccagggctgttaccgttgttttcttgaatcactcacaatgagaaactaacattttgctttttgtaataaagttaatttatattcagt

SEQ ID NO: 81 - DNA, 3818 bp, Homo sapiens
gcgggagcgcgcacgctcgcgcacccggatcccggctcctgcatccagtcgccattcgggaggccgctgcgctgcagggcctcgcggagccgcccgcgaccgcgagccgggccctccgcgcggtccatcgcccactggacgccgcccgcggccggaccggttcaacttctcatctttgttcttcttcatatactataggctgtttgctgtggtttagtcaaaaagccatgtagaatgcctgccttttgaagaccacttttaaggtgtctagtaagacagcagcagtattgaaagtttttaaagaatataaccgtgtgtgttggtaacagacagaagaatggaagcattccaggaacttcgtaaaccatcagcacgtttggagtgtgaccattgcagtttcagaggcacagactatgaaaatgtacaaatccatatgggtaccatccatccagaattttgtgatgaaatggatgctggtgggctaggcaaaatgatattttaccagaaaagtgcaaagttatttcactgccataaatgcttcttcaccagcaagatgtactctaatgtatactatcacatcacatccaaacatgcatccccagacaaatggaatgataaaccaaaaaatcagttgaacaaagaaacagatcctgtgaaaagccctcctcttcctgaacaccagaaaataccctgcaattcagcagaaccaaaatccatacctgccctttcaatggaaacacagaaacttggttcagttttgtctccagaatcgccaaaacctactcctcttactcccctggagcctcagaaacctggctctgttgtttctcctgagctacagacacctcttccttctcctgagccttcaaaacctgcctctgtttcttctcctgaacctccaaaatcagtccctgtttgtgagtctcagaaacttgcccctgttccttctccagaaccacagaaacctgcccctgtatctcctgagtcagtaaaggctactcttagtaatcccaaaccccagaagcagtctcatttcccggaaacattggggccaccttcagcctcatctccagagtcaccagttctagctgcttccccagaaccttggggaccatccccagctgcatctccagaatctcggaagtcagcccggactacctcccctgagccaaggaagccatccccttcagagtctcctgaaccttggaagccgttccctgctgtctccccagagcctaggagaccagcccccgctgtgtcaccaggctcttggaaaccagggccacctgggtcccctaggccttggaaatccaatccttcagcatcatcaggaccttggaagccagctaaacctgctccatctgtgtctcctggaccttggaaaccaattccttctgtatctcctggaccttggaaaccaactccatctgtgtcttctgcatcctggaaatcttcatcagtctcacccagctcctggaagtctccccctgcatctcctgagtcatggaagtctggcccaccagaactccgaaagacagctcccacgttgtctcctgaacattggaaggcagttcccccagtgtctccagagcttcgcaaacccggcccaccactatccccagagatccgtagtccagcaggatctccagagctcagaaaaccctcagggtcaccagatctttggaagctttctcctgatcagcggaaaacttctcctgcttcacttgatttccctgagtcccagaaaagttcccgtggtggttctcctgatctctggaagtcttccttttttattgagcctcagaaacctgtcttccctgagacccgaaaaccaggtccttctgggccatctgagtcccccaaagcagcctcagatatctggaagcctgttctctctatcgatactgagcctagaaaacctgccctgtttcccgagcctgccaaaacagcccctcctgcttctccagaagcacgcaaacgtgccctttttccagagccccggaagcatgcccttttccctgaactccccaaatctgctctattctcagaatcacagaaggctgttgagcttggtgatgaactacaaatagatgccatagatgatcaaaaatgtgatattttggttcaggaagaacttctagcttcacctaagaaactcttagaagatactttatttccttcctcaaagaagctcaagaaagacaaccaagagagctcagacgctgagcttagtagtagtgagtacataaaaacagatttggatgcgatggatattaagggccaggaatcaagcagtgatcaagagcaggttgatgtggaatccattgattttagcaaagagaacaaaatggacatgactagtccagagcagtctagaaatgtgctacagtttactgaagaaaaagaagcttttatctctgaagaggagattgcaaaatacatgaagcgtggaaaaggaaagtattattgcaaaatttgttgctgtcgtgctatgaaaaaaggtgctgttttgcatcatttggttaataagcataatgttcatagcccttacaaatgcacaatctgtggaaaggcttttcttttggaatctctccttaaaaatcatgtagcagcccatgggcaaagtttacttaaatgtccacgttgtaattttgaatcaaatttcccaagaggttttaagaaacatttaactcattgtcaaagccggcataatgaagaggcaaataaaaagctaatggaagctcttgaaccgccactggaggagcagcaaatttgataacacagtgtgaatatttgttctacaaaggtgtttgttggaaccattctttgtaagtatagcttatcagatagcatagttggatcagtagatgacatgtatggtgtaccgtgtttcactgtctcagttgtgttactaagaatgagcatttgatcatttttttctggtctctgtctatgtgactatcttgtaagtcaataaatttctgtatagtccagatggattaaacttctcatttcttttaaatatgtatgaataataatacaaggaagtaggcattccatttaataatcaagagcaagttgtactcaaagcattcagttaaagtgtatctgtgtgtggaactaatttcagacaatagaaaatattagttgaaatgtttaagaattaggcatgaaaaataaatttgagaaattttgtttccttacatgtatttttaaatcataagagttattttctatctgatgtaaaattagtttataaatcttaatcagcttctagatgtttattagcttttatgtcatgaaatgttggagtctcagggttgctgattttctgctaatgggaaaaattgactaagtctttaaaatagtttgcagccttctcccacaggagacaagtgaaagataagtgtgattttagatctttcttgtccatagttgttttcagtggagtcttccattctgtatcttaccctaagatctggttcttccctccccatccccaccccccaacccaccgcctgccagctcacactaatagatgattcttaattgccaaatgtgttagagtttgtatatcctactcctgggccttacatgtcgcctgttggggcttaagaccaggttgataagtaggaactgaaagtcttccagattcacagtagaaaattttatagacatttctgttaaagaaatatatcgattttatgtttttcaattatgttactgtaaataccttgtacctgttcatggattattttattctaaaatattttgtcaaatgtgtatcaaccaaattaaaaagaaaggttttcatgtca

SEQ ID NO: 82 - DNA, 2900 bp, Homo sapiens
ccgatctcggcctcagcgtgagcatgcgcaggtccccgccctcgctgcgtttgccttgagcgcgataatttggtggcggggtccggcgggtgctggtttgttctcggtgaacggcgcgcggggtctctcctgagtgcgagctacgggaccttcgccatgccggggatggtactcttcgggccggcgctggccatcgccagcgacgacttggtcttcccagggttcttcgagctggtcgtgcgagtgctgtggtggattggcattctgacgttgtatctcatgcacagaggaaagctggactgtgctggtggagccttgctcagcagttacttgatcgtcctcatgattctcctggcagttgtcatatgtactgtgtcagccatcatgtgtgtcagcatgagaggaacgatttgtaaccctggaccgcggaagtctatgtctaagctgctttacatccgcctggcgctgttttttccagagatggtctgggcctctctgggggctgcctgggtggcagatggtgttcagtgcgacaggacagttgtaaacggcatcatcgcaaccgtcgtggtcagttggatcatcatcgctgccacagtggtttccattatcattgtctttgaccctcttggggggaaaatggctccatattcctctgccggccccagccacctggatagtcatgattcaagccagttacttaatggcctcaagacagcagctacaagcgtgtgggaaaccagaatcaagctcttgtgctgttgcattgggaaagacgaccatactcgggttgctttttcgagtacggcagagcttttctcaacctacttttcagacacagatctggtgcccagcgacattgcggcgggcctcgccctgcttcatcagcaacaggacaatatcaggaacaaccaagagcctgcccaggtggtctgccatgccccagggagctcccaggaagctgatctggatgcagaattagaaaactgccatcattacatgcagtttgcagcagcggcctatgggtggcccctctacatctacagaaaccccctcacggggctgtgcaggattggtggtgactgctgcagaagcagaaccacagactatgacttggtcggaggcgatcagctcaactgtcacttcggctccatcctgcacaccacagggctgcagtacagggacttcatccacgtcagcttccatgacaaggtttacgagctgccgtttttagtggctctggatcacaggaaagagtctgttgtggtcgctgtgagggggaccatgtctctgcaggatgtccttacggacctgtcagcggagagtgaggtgctggacgtggagtgtgaggtgcaggaccgcctggcacacaagggtatttctcaagctgccagatacgtttaccaacgactcatcaacgacgggattttgagccaagccttcagcattgctcctgagtaccggctggtcatagtgggccacagcctcgggggcggggcggccgccctgctggccaccatgctcagagccgcctacccgcaggtcaggtgctacgccttctccccaccccgggggctgtggagcaaagctctgcaggaatattctcagagcttcatcgtgtcactcgtcctggggaaggatgtgattcccaggctcagtgtgaccaacttggaagatctgaagagaagaatcttgcgagtggtcgcgcactgcaataaacccaagtacaagatcttgctgcacggtttgtggtacgaactgtttggaggaaaccccaacaacttgcccacggagctggacgggggcgaccaggaagtcctgacacagcctcttctgggggagcagagcctactgacgcgctggtccccggcctacagcttctccagcgactccccactggactcttctcccaagtacccccctctctaccctcccggcaggatcatccacctgcaggaggagggcgcctcggggcggtttggctgctgctctgctgctcactatagcgccaagtggtcacacgaagcggaattcagcaaaatactcataggtccgaagatgctcaccgaccacatgccagacatcctgatgcgggccttggacagcgtggtctccgacagagcggcctgcgtctcctgtccagcacaaggggtctccagtgtggacgtggcctgaccagggccactggaaactgtcccaggaacgatggactcacgcttttgtccttaaactgacttaccatccgaggagttcccatgacgccaaaacagcgaatgtccatcaacaggaatcggatgggaacagaattccatggtctcaatgacttaagtttatgggaagtcattgtggccataatggtagcagaagtagtgagcacgctcaggtgataggacgactcctgagacccagcgaccgtggagacagcctcgggaagccctggcccgtggatggatcccttggctgtctgaggactgctccagaagtgcgggaatccagggcccacccagaagaccgtgaacagttccttagcctcccacccccaaggcagctcttttcatccaactcagtttacaggcgtggtttgtttttcaaactgggcttcctggatgtacaaatggaactgtggtgagggtgcgggctggggttttctcctgggcgtcaccaagggcagccctgggctctggctggggatgaagacgaaacccgatcgggaaagtaagtggagcccccggccccgccgagccacagccccccaactgcctattcccactgcccagttgtttgtccacatcaggagttgctgattgaattcttgctactcttctggctctggggtcggccagtggattcaggagttgaaacaataaagcgcgcgtcaccatagtgcttgtgtgtacagc

SEQ ID NO: 83 - DNA, 635 bp, Homo sapiens
aggaacagacttgtttttgacttgtctatcttttctaaggtttttttcatcagatacaagttcttccagttattgacacagtcactcctaagacttagcttaagtgttaatggctcaacaatctcagccgattaacactaatcataataatatttattgaatgtgtgctttgtgccagccactttgctgagcccttttcatgttcttaaccctcactaactccaataaccagagtatgattttgttcagtgaaacctgagattgtttctagagtaatcagatagtattgagtagcagtgttatccccaatagtagaagagagccaaggcttcagaaaattgaagaactctcccaaggtcatagagttggttaaggagagggcctctgttgtatatccagatggttgactatgaacccacattcttaattagattaagagtagtagaactctttctctgcctagctcttgttgatcagtagaaaattcactcagggctgggcgcggtggctcacgcctgtaatcccaacactttgggaggtcgaggcgggcagatcacctgaggttgggagttcgagaccagccttaccaacatggaggaaccctgtctctactaaaaatacaaaattagctgggt

SEQ ID NO: 84 - DNA, 1046 bp, Homo sapiens
ctccgccagacagaggtgctggggctgtgcaggaaacgaagtgattagaaatcccggataaacacacaagcaggcgttgtcatggtgactgggaaaaacacacaagctggcgttgtcatggtaatggagtgtaggacaggcctggagcccctcggtctcttgctggcggctggcacagagacgggctgccgtgggctctgaccttaataccgggtcacagtcgcttctaggaccaagaggacagagaccccatcaccgtatgcaggggcctgtttccaggcagactgcccagtgcccagctgagcctcgggtgcagtgcgacccccgcagggcatgtccagaccccaggaccccctctcaggtctagaagatccagttgggcagtgttggtaccaccaagagtagacaggacagaggatcagagacaatcccacccagcaggacccaaggactcaggcagtggcttttcaggtgtgtgggccgaggactggggagtcggtgaattctggggcccctggggtggccgttcaggaactgcagcagctccccccaccacagatgctcgctgcctactgaagcggccacgtgtttgaatgaagagcagttagaggaacgcttgcaagagaatgtgtttattacctgaggttatgacaatacagaacatacaatgttttctgtggaaaatgtgatactacagaggaaaaggtcactttaattaaatggcaattagaagtaacagcattgcaaggtggggtgcagcagctcacgcttataatcccagcactttaggaggctgaggcgggtgggtcacttgaggtcaggagttcaagaccagcctgggcaacatggtgaaacctcgtctctactaaaaatatagaaattagccacgcgtggtggtgcgcgcctgtagtccaagatactcaggaggctgaggcaggagaacctcttgaccctaagaggcagaggttgcagtgagccaagatcgtgtcactgcactccagcctgggcaacagagcaagactctgtctcaaaaaggaaaaaaagaa

SEQ ID NO: 85 - DNA, 284 bp, Homo sapiens
ttaaaaaccaggggcggtggctcacccctgtaattccagcactttgggaggccaaggcgggcagatcatgaggtcaggagttcaagaccagcctggccaacatggtgaaacctcatctctactaaaaatacaaaaattagatgagtatggtggtacgtgcctgtaatcccagctacttgggaggctgaggcaggagaatcgcttgaacccaggaggcagaggttgcagtgagccaagacagtgccgctgcactccagcctggtgacagagcgagactccatctc

SEQ ID NO: 86 - DNA, 1632 bp, Homo sapiens
ctaatgaggagtgatgctgagcatcttttcatatgcttactggtcatttgtatgttgtctttggaaaaatgtctattcaagtcctttgactattttaaaaattgggttattagagttatcgttgttgttgacttgtaggagtttctttctatattctggatattaatcccctatcagatatatgatttgcaaatatcttctcttattccataaggttactttttcactttgttgattgtgttctttgatgtatagaagtttttagttttgaaatagtctaatttatctgtttttacttttgtggtctgtgcttttggtgtcatatccaagaaatccttgccaaatccaacgttataaggtacttttaaggtattttagttgtcttagtctatatttctgtactcacctttctttatccactcatcagttgatgggcatgtaggttggttccatatctttgcaattctgaattgtgctgtgatcaggtgtctttttagtataatgatttactctcctttgggtagatacccagtagtgggattgctggatcgaatggtttttataattttctattttaccacagtttctctctgcatttttcctctttgaccactaaccatgtgaaattctcatattgacctttataatgatcatgaactcttagtatcattgggaaggccacatttgccacttatgattgtaaaccttatcctccatttttcctgttattgttggtgcaaaaagcacctattataccaggactttaaaaatcagtctgataagtctttgataagtctaataataataactgataagtccattgaatttgcttctgattactttttctttagtagctaaacatgtatgtggatctatttctggaaattttaggctccagtttttgttgttgttgttaataaaatgcaatggaatgtaatgatcatcacttttcattatgctttaaaatctggtaaatggaggctagaacactcctgtaaggcaagaatattctctctgttggaactcaaatacacagaactgggtaaatctcaatcttaatctttgattcaggacacaacatggctctcttttacttgctttctttaattgttttttaataatgtggtaagcatttctgaatctcctatccaatacaaaaactaggacaatacagacagtaactcctatggttacaatgaacactcctctccacttaaattaattatttacactgatgaaattgaaatagcaaaattttaatgactaaatactgtctttgattttttgttccaggtctgtcaatattaacttcttataattttcttcaatggctttgggggtacaaatggcttttggtcatatagatgaattctacagtagtgaagtctgagattttactgcaccggtcacctgagtagtgtacattgtacccaatatgtggttttttataccttgcccccctcttaccctccccactttgagtctctagtgtccattatgtcactctgtataccttttggtacccatcagttagctctcacttataagtgagaaccaccactgtatttggttttccattcctgagtggcttcacttagaataatatcctccagctccatccaaaattgctgc

SEQ ID NO: 87 - DNA, 480 bp, Homo sapiens
atgatacaagatggtcatcaaatacttcctattgtagttgaaggtcaatttctctttccacttttcctcaagatgcagaatggctggttttttcttttacggtttcaaaacttcactagttagagataatactactgctaaattttaatctattatatttgcatcacaacttttattatatcaaagtaattgatacagctatctatacttgtcttttgtctaggatatgctctccaaatcttgattctctttaaaagataatggcatacttcctaacatatccaacttaacagcatcaattttaaatgctggaccctttaagtacttatgcattatatttataagaacatgtctatggccgggctcggtggctcatgcctataattccagcactttgggaggccaaggtgggtgggtcacttgagtccaggagttcaaggcaagtctgggtgacatggtgaaaccccatgtctacaaaaa

SEQ ID NO: 88 - DNA, 1583 bp, Homo sapiens
gggccctcataataagcattgttactattggaagttgttttcacattctttccaatattaaatatgtatttttttaagtaatgataatattttccagtggctcatttggatgagaactaccctctatttttaatattaaaactacatccaactcatcatttagcctttggttgtacagttgtgtaatgggctatggactgttacacaccttaccacctctaggcctatgttttttctttccccatatattctgatggggataaatactgttttgcctctcccataggaatggaatacatttattctaaaatgatctttcacagaagtaagagagagggaaacctaaatatacctctaaattgtttgaagttggtcccagcagcataaaatgggttggccccaaagggttggagggtgggcttggttatcagtatttgttttcagaatgagatgggagcatctttcctttgccacgtgctttgtgcttgataacatcatgcttggttcaaacgacaactcagcacaaagccttgagtataaattgttggaatcaaaacatctcattctgatgacgtggtttaattttttaatttttttttttaataggggtgggagggagggtactttgccccagaagggagggtgtctgcactaaggatttagaaacactttggaagctcataacctcatcagaaactgcctttagccacactcctgaccttctagatgagtaacaaaaaaatgaaataagttcttggaaattaagccatttattttaatttgctatttttttcaatgttctaggtatctttaaatttgttattgtggaatcattttcctgccagatacctttatcaaaattattggcctcatgagagctgaagtaagtcagctttttggtgaactttagtggacttctgtgagattgtagttgtactttgtatctctaaatctaaagatagttttttaaaactcccaaagaaaatctgctctcctttctgatctaaaaactcatctttggggtaaagagttaagtgtccaaaggttgtcacagttcatgaggtcagagggagctagcctggcacctggactctgcccatccacagctgacagattccaacagaagtgtatttaaattctccagtagacaatgctgggtaagggagggggtagggctgggttattaagatacaggctgctgtattttacattggttgtgggggaaggggagcctggagaaaacaaagtcactattcccttttttgaaacaggaaaaaaaattattttttgttcagtaaaaatggtagagaattccaatgtccctagccacaagggaccagttccactgagaagtgaacagtgggaactcaaaatttcagaaacattgggggaagggaaaattggctttctcttaattggcagatgttccagtggggggggggggggctctgtttttgttgggatgtgttatgttgtatgtacgcatatatggaccggagtctgctgagtttataaggttccaaaaatatggtaaaatcttggtttttgttaatttatctcaataaaagcccactggaactcca

SEQ ID NO: 89 - DNA, 742 bp, Homo sapiens
aaaattgtcaatgtggatgattctttaaaccataatttgggccaaaagctgagcatcacaccaagaaaatatctctgcttctagacatcaagaaagagaggtggagataaaggaaaaaacttaatcccgaattgataggagtgagagacaacaaaccttaggacagggaattcttaacttgtggcagagcaaacagtagaaactcatgagacgtgttatccaataatagaaaataggaacatgagatttattccactagacagtactaggactctacatgtaaactcatgggaattgaaataaagttctctgctgtaattggagcaagatagactgaggagagagtaaaccacgaatgctggctcaagacaaaaaacctagcagaggtgcattgcagacatacccatgaaggaaaaacttacacaaggtcaccctaaaggaaggacattgttaagccctttgaaataatggggtggagaggaaaatgaactgaaaaaatgaaaaacacccacaggaagaaatcaaagacgattgtgtcaaccccagggctacagaagtgaggaataaaattggctatttccggacactgactttcttgatttgttgaacatacgtgaaagcaggacatgccatggtcgctggttgcatcaaatagaaatgactcattggaatgttacctccaaatccttacatgaagagtaagcaaaagatgaaagcttttatgattcctttagaaaagaat

SEQ ID NO: 90 - DNA, 2729 bp, Homo sapiens
cttgcgcactcagtcaccagcccccttctggggtccaagctgtgtccccttctctaaagaggtaagccctgagtcatgggaagatggaaaccggggctcatgagacaggatgttttttaagcaccgtggtgtcttgttgacttgcacatgcacgggggtcttgggtaaccacagggctcagggtatttgcaggaacagttcaagtgctcacttgtcttggggctgtttatggggaagtggtttccacagtgagaggaggtgagatattgttgtcaccccggaccacacttagctacttccttctcactaaagctctgtagtcatattttccctggcagagcagaaacttctatgttatcccacagctgttctaacggtgtagacttgacttatgcaatgatgccaggagtcctgagcagcacagcccaacttcaatcacacacagatggacagagctgtattagcaaagcctgagctactgagcgatgagagtacagccaggctttcagacatctgttcattcaagagagatatgcgctaagccaaggacctaaagatgtgtttaatatgggtgctaatatgcataaggaaccttgaaataaatgttcttagcctttggccaagagggtccatgtctaggaatctattctccatagaaataaattcaaatatggaaaaaatgaacaatgcataagtgtatttggtccccagcatatttatagcaacttaaaattggacccaatttaaatgcctatgatatggaaatggctaagaaaattatgggatcttcccttgattggctattaggcagcctttacaaacaatgcagtgacatgagaaatgcttatgttatggtaagcttaaaaaactcaagatgcaaatcagcttattttaatcaggagccacctagcatttgggatgtggtcaatcccacataatgtatttttgtgggtgcagttcccaggaaagaggaggaataaaaacggcaagtatgaagtgtctccttcgcttgcagtctccttgtctacccctttgtccatccactatgaaaggactcccttctgttccttaatatggacaatttctattgaggactcattgttctaagaattgtctcatctcctcctgcatcctcagtgcccgatctttggcttctatgaaggaaggtgggtagtgcgtatggcaggtccagttctacctttcttagtatgttctggcgtgggtatgtagccccattttctagtggttaccttgacatcatgaagagtttatgtctcttttgccctaggtttgggcaatagtcattcactgtgcaacaggaaatacacgagtcagcatcttattaaaaataaagtcattcaggaaagtggacgacagtttctaatctagagagcataggagaagaaatgtttaccacacacaaagtattagtgccttttatatcacgaagacaaaaataacaggaaaaagacaaacacattatagtgaaaacttgtttttcctaaccagcatctattctgcatgtttcctgatgcccgaaactcacatttcctcaggaaaatctcccttctgcaccattctcaggctttaagtttatgtaaaattcagtaaacccaaagattcaagttatgtgccttgattaacttaagcaaatcaatgaaacccatccccataaccacagcgacaggttaggaaattcggttcctaagtcagtcacatccgaaagggcctagtgatgtttttttccagtgggatcacagactcactcttccttgcagaaaatgaacaaaggattcatgtaacactggcaggtactggcagccacccagggcctctcacaggaaagggagatcagaaagagaagcaaagaggactcatgagataccatagggctgctgcgtccagccttgcctggagctagggccacctcgatgccctatagtcttggagccacaacgtgcatttactcaaagcctctttgagtttggtttgcttgtttgctttctgcctggaaactgccagcatcctgagagatacgagatctgcatctgtgcagagacacagggtttgttaaaagtcacaggccctgactgaagtgtggaactggctgaaatgagaaagtggtaatttggggaggaccttgtgaaatggaaggagttttaaaccttacatgcatcagaattacctggagccttgtgaaaacacaggttgctgggccctagtccattaagaaaggaagtggggcttagaatgttcatttctcccatgttcccaggtgatattcaccatgctgtcctgtctgggcactaccttttgccatacccattacaaggtattgcacgtgctggttgaactatggtctgtcttattttggtgctaaaagcctgtgccaaataccaacgctgcagcattaaggaatgtgatagaaaagattctgaatataggccaggcgcagtggctcacgcctgtaatcccagcactttgggaggccgaagcaggcagatcacgaggtcaggagatcaagaccatcctggctaacatggtgaaaccccgtctctactaaaaatacaaaaaattagccgggcgtagtggtgggcacctgtagtcccagctacttgggaggctgaggcaggagaatggcgtgaacctgggaggcggaacttgcactgggctgagatcgcgctactgcactccactccagcctgggcgacagagcaagacttcgtctc

SEQ ID NO: 91 - DNA, 470 bp, Homo sapiens
aatgaattccagaatccggggcaggttggtaggtcccaatcccaggggaatgtggtaaaagtggtacccggttttgggatcggaagggtccaataaaatccttatttaataattcggtacccgaaggccagtgtaatcccaaaaaggaataaaaaccaatagttttggtggcttccgccggaattttaaaaaatggtttttaaaataaaaaagttaaggtcccttttaggtaattatttttaagaccaattgccaaatatccacccggtaaacctaataaaaccccccccttcttaaataccatttaaccattgggcaaaggtccattagggtgatttggcccgattaaaatatttttaaagacctaaaaaaaatgccttccggtttcccggccattaggcaggaatttttaatgattaccccataagcctaccatttttttttttacccccaaaaataaaaattgtgaa

SEQ ID NO: 92 - DNA, 597 bp, Homo sapiens
attacaggcgtgagccaccaagcctggcctaaaacatttaaaaatgtttattttaaacatacataagacatgcacacataaagatacgcatagcatgattgagggcttggtgttttgtttctgtaacactggatttgaaacgaaactataatgagaatgtatagcagggctgggcgaatgacaggcttgcttatgactggagggtcaagggctattgagtgcaaaagctggatgtaatcagattagctcagtgttttgtttttatagctatgcattttagcgtttaaaccatggtaaagaacagcttttaaaaaaaaatcgcttctcagccttttggctaagctcaagtgtaaaaaaaaaaaaaacagctttaaatctcaagcttttgcccctaatcttttaaaatttcattgaaataattatcagtttactgtttcactgcaccacaaatttagtttcaggtgtatcttgaaactcattgatatgctaataagttttattaaaattgttaaattccttcctatgaatatactttttatacagatgtgacttaagtatttaaatgttttacttattcacaaaataacaaagaatggc

SEQ ID NO: 93 - DNA, 1140 bp, Homo sapiens
aattttttgtatttttagtagaaatggagtttcaccatgttggccaggctggtctcaaactcctgagacctccacctgcctcgacctcccaaaaagctgcaatatcaggcatgatccatcgcacccggccacccatgtattcttgattgaaaacatttgctcatgtcttagttctacagctgaccttctttcactgttttcaaggtcaataactgtgtgttcacacttctgcattttataaatgttactgtgattttcttgtaatgaagaattaaatgttgggagtcaatggcatcagaaccttgcaaaagaggtttttttagcccaggtatgtggaagacacttctttaattttcaataatgggtgtgataaagaccaacccttcccattagcccttccaggcccacatgtaagaattcagacacatcttttcactcatctcagaccttctcagggtaactcggtgaaaatgtcttcactctgagcctcagtgagcctccctgcaacttgcagatgaggggctagaccggaaaagctcaacctgagtgaccctggcccctgaaatgattggcaaaatagagtgggtgtctggatgtggctttttttctgtgagaggggactgtccagttgtaattagaattttaaatgggatgcagtaccctaaaaatgaaaaaaataaaaagaagaatggaagaaacagagttgtagactcagacacagagaccatcttcggggcctttctctgtgtgaggacatcacagcgaaatctaaagcaggtcatgtcagtccctggcagggaaccctccaccagcttcccgtgttccccaggacaaaagccccactcctcactgtggctccacagccctgtgtccagggcccctgccagtgtccagcttcctcctgggaacttgccctcatctcatgactacctctgccccagtcacagttgcttttctcttttcccaaacatcaaaacccttcctgtctcaggttattgtccctgctcttacactatgtacctaaatgatgacagcactgtccctttctcctccttcaggtctaggctcagagatgtctcccatgccctcccacccccatctgaagatycctctgcctgtcagtctctcacgttactcagg

SEQ ID NO: 94 - DNA, 520 bp, Homo sapiens
agaagtaaaattatctcaattcacatttcatttatgactttattgatagaaaaccttaataatacacatacacaggattctataagccttaataaagaagttcagcaaagtagcagatacaagctcaatatgacaatcagtttaatttctgtacaatgatcatgaacaatctataaaagaacaatttcatttataataacataagcaagtgtgtaagtatatagttaacgaaggaagtgtaagatataaaacattgtgagaaattaaataagaccaacaaatgaaaagtcatctcttattcattgattggaagatataatgttgttaagatagcaagccactaaactgacctacagattcaatgctatccctaatcaaaattgcaacagcctttttggcctacaagctgctcttgaaatgcatataaaaatacaagggactgaatagccaaaacagtttctaaaataaaaacaaaattgtaggactcagatgtctgatttccaaacctaatacaaagctg

SEQ ID NO: 95 - DNA, 501 bp, Homo sapiens
ggtatattttatgtgctgagaagtgtcaatctagaattctgtagcaaacaaaactatcaggaaaatgggccaaagacattttggataaaaagagtttactaccaacatgtcctcattaaatgaacttaggaaagtttattccaggaatcagaattaagatcagaaagamcatgtaagacgtaagaagagatggtgagcaaagaaagtggtaaatgaggccaggcacagtggctcacacctgtaattccagcactttgggaggccaaggcgggcggatcaactgaggtcaggagttcgaggccagcctggccaagatggcgaaacctcatctctactaaaagtacaaaatttagccaggcgtagtggtgcttgcttgtaatcctggctacttgggaggctaaagcagtagaatcgcttgaacccaggaggcagaagttgcagtgagctgaaattgcgccactgcactccagagcctgggcaacaagagcaaaactccgtctc

SEQ ID NO: 96 - DNA, 1760 bp, Homo sapiens
gtgctggttcagggggaaggaggagcacaaagtgcaaagggctttctaccagtgtccagtgtgtttatgaggaggcacattgaccattgtcccttatgtctgcattttcatttactgtgctgtgtatatagtgtatataagcggacataggagtcctaatttacgtctagtcgatgttaaaaaggttgccagtatatgacaaaagtagaattagtaaactactacattgagtacactttgtgttaaaattcatagggaagacttcttaaaaacaagtgaaattgttaaaaccccccctaagcattacagatggcttatagctgtccacggggttggtagaggtgggaaagggaagggttctaggccagaatgttcctatttagaagacactcaaattacagtctgtgttatgtatgtataccatttattcaatgctactgtgtatataatggaaaacttaagtcctggcgacagagcgaggctccgtttcaaaaaaaaaagtgcacaatgtaggttaacagtagagggcttaagtaacacccctctaagcatttgttttcagtacttcctaggagtggttgcatttgggaatggaattgttaaaacttgatgcttaggagcgaatgcagactattcattgggtgtttggggtgggggaagggggggtgggcagaggaggtatgcagggagaggggttctgtgctcctgagattagttcagatggtctaaccattgttctatatgtgcattttagttaatattgtgtattaaaggataagtcttaatgctcaaagtatgttaaaaatagatgtagtaaatcagtccctttgtgaatgtccttttgttagtttttaggaaggcctgtcctctgggagtgacctttattagtccaccccttggagctagacatcctgtacttagtcacggggatggtggaagagggagaagaggaagggtgaagggaagggctctttgctagtatctccatatctagacgatggttttagatgataaccacaggtctacaagagcgtttttagtaaagtgcctgtgttcattgtggacaaagttattattttgcaacatctaagctttacgaatggggtgacaacttatgataaaaactagagctagtgaattagcctatttgtaaatacctttgttataattgataggatacatcttggacatggaattgttaagccacctctgagcagtgtatgtcaggacttgttcattaggttggcagcagaggggcagaaggaattatacaggtagagatgtatgcagatgtgtccatatatgtccatatttacattttgatagccattgatgtatgcatctcttggctgtactataagaacacattaattcaatggaaatacactttgctaatattttaatggtatagatctgctaatgaattctcttaaaaacatactgtattctgttgctgtgtgtttcattttaaattgagcattaagggaatgcagcatttaaatcagaactctgccaatgcttttatctagaggcgtgttgccatttttgtcttatatgaaatttctgtcccaagaaaggcaggattacatctttttttttttttttagcagtttgagttggtgtagtgtattcttggttatcagaatactcatatagctttgggattttgaattggtaaatattcatgatgtgtgaaaaatcatgatacatactgtacagtctcagtcccataaaattggatgttgtgcctacacacagga

SEQ ID NO: 97 - DNA, 217 bp, Homo sapiens
aaagtagtcattcttcactgagaaggaacacataccaaggttagtgggttcgatcatttgaaaaatggcagcaccattcattttaaacattttctggctttttactatggaatctctcatggtataaaaataaattttagatttttcagagccaaaatgaaaatactttagaacaaaatcaggccaaatctttggaattcaaagtggctgaacacct

SEQ ID NO: 98 - DNA, 1311 bp, Homo sapiens
ggtacctgaaagaaaatcaaataggaatgacagtatttagtgtatggccagtggtttacttagtaactggatgaacagactagagttacaggttttgttttgttttttttttctattccagtagtatatctgagtaaatcctgtccctcagtagatcatctcttgggatctggtttcttgatctgtatttcaatatattctatattccatatagatcaagactttctaacataaagcagtgtggaatagacttactttttatcttctctgttactcttttgatttgtgacttttaccaatttattgaacttcttaagtgtcagtgtttttaatccattaggttatcgccaaggcctctaaaagctctaagattcagtgatatgaatacatatttgcagtattagagacattgtactgttttcacttggcttctaggacattagattttctattctccctttcctatgctcactcccagattccttaaccagttccttgcatctttgtgtattagaatgcctcagggataagtcttggatttctgctcctttctagctgcactcacttccttggtaagctcatctgatttcatcataacttcacctttacatactgcaaactcacaaattatcttccctgaacttgagactcctatcctgctgcctgcttatcatctttacttgactatataacgaacatatcaaacataaactgaactgatagtctcctaacctgaaacctgcttctatagtcttccccaactaagttattggcaaatacgtccttgcattttctcaggccaaaatcacatcatgatccttggcatttctttctctggtaccccatgccctgtctgcagatctattggcaaaacctcccaacatcttaacagcagctttactaccacacttttccaaacggattacctctagcctgcatgattgcattagtctgcctccctgcttctggcttttacctactcaggctattcccagcacccagaatgacaactttgaaaacaaagcttgccgccacgtgcagtggctcatgcctgtaattccaacgctttagaaggcggaagtgggcagatcgcttgaggtcagaagtttgagaccagcctggccaacatggtgaaaccccatctctaccaaaaataaataaattagctgggcatggtggtgcatacctgtaatcccagctacttgggaggctgaggcaggagaatcgcttgaacctgggaggcggaagttgcagttagcagagatcatgccattgcactctagcctgggcgacggagtgagaccccatctc

SEQ ID NO: 99 - DNA, 144 bp, Homo sapiens
ttttaaggaaaaagtgacctacatttcatgaagcaaagagatacagccacacacaggagccgtttgttttaattagattgctggtttccctggccaggacccaaaaccactgtgtttccccatagatacaattgacaaataaaa

SEQ ID NO: 100 - DNA, 528 bp, Homo sapiens
agttcgagaccagcctggccagtatggcgaaaccctgtgtctactaaatatacaaaaattagctggagatggtggcaggctcctgtagtcccagctacacaggaggctgaggcaggagaatcgcttgaacctgggaggcagaggttgcagtgagctaagatcgtgccattgcactttagtctgggcaacaagagcaagactccgtctcaaaaaaaaaaaaaaaaaaaaagcccacaaaaaccagcaaaaaatcctcggccccatcaccccagttgcctcaccaacagcctctcccagaccaggaagctgtttttattttaacttcatgcaaatgttgctaatacaagatatattcatttttttaacttacccttttttacaaaaaagatggttctgaaattgaactgtatttaatgtctttaatggtgaaaaaaggaaaagtcatagatgacatgtcattattttgtaaaataataagatcatggtctggtactcactttggcagcacatataataaaattggaaaga

SEQ ID NO: 101 - DNA, 1287 bp, Homo sapiens
aggtagtattctgataattttggactcatactcaaattcacaaagttttgaaaagtcattgtgaatacattaagagaaataacagaatctgacctgcaaagactgcagattttggaattactggattagagtattcaaagacacacaaaattttttttaacaactctaaaattcggatgacagtgcagcattaaattgacacaaaatgatgtgtttttcagtacttgatggcttagatttattgaaatacagtatgtgtaaggaatgaaagaatatccaaagattgagaaggaacaagacaaaaaatggtgggggtggaggataagcaagagcatatgacaagaaaaataagatttggaaacagtgaaacatctagacatgaaaagcaaaacagataaaatgagccagaagaccaagatgaagaaattatgtagaatgtatgaaaactcaacaccagggacattttgaaaggtcaatgaacatctaatagactaccagaaagagatgatagaatggtttgggatgatatttgagggtattttagctgagaaattttccaatttgatgaaagtcatccttgcatttgaggaatcaagaaaataatctgtagacccattgagctaatttgtagatgacagacaacgtgggaaaccagagtggtgaaactcttgataagcaaaccactaaaaatagtctctaaaagagcaagagaaagaaagcattatctacaaagtaacagcagttagtgtgacagctacttgataacaatgaaaaacagaggagagtggtatattttatgtgctgagaagtgtcaatctagaattctgtagcaaacaaaactatcaggaaaatgggccaaagacattttggataaaaagagtttactaccaacatgtcctcattaaatgaacttaggaaagtttattccaggaatcagaattaagatcagaaagaacatgtaagacgtaagaagagatggtgagcaaagaaagtggtaaatgaggccaggcacagtggctcacacctgtaattccagcactttgggaggccaaggcgggcggatcaactgaggtcaggagttcgaggccagcctggccaagatggcgaaacctcatctctactaaaagtacaaaatttagccaggcgtagtggtgcttgcttgtaatcctggctacttgggaggctaaagcagtagaatcgcttgaacccaggaggcagaagttgcagtgagctgaaattgcgccactgcactccagagcctgggcaacaagagcaaaactccgtctc

SEQ ID NO: 102 - DNA, 3670 bp, Homo sapiens
gcggccgcgatccccaccacaccaccagcccggccgcacggggcactgagccgggtgctgagcaccggaggccccgccgaggccgggactcaggacctgcagagaaacgcctcctgattttgtcttacaatggaacttaaaaagtcgcctgacggtggatggggctgggtgattgtgtttgtctccttccttatgccctttattgctcaaggtcaaggaaacttaattaacagtcccacaagccctctagccataggactgatctacatcctcaaaaaggaagttgagcaccattacaaaaaaggagaaatgaaggctagcctattcataaaatcaccttacgcagtacagaatatcagaaaaacagctgctgttggagtcctgtacatagaatggctggatgcctttggtgaaggaaaaggaaaaacagcctgggttggatccctggcaagtggagttggcttgcttgcaagtcttggatgtggtttattatacactgcaacagtgaccattacgtgccagtattttgacgatcgccgaggcctagcgcttggcctgatttcaacaggttcaagcgttggccttttcatatatgctgctctgcagaggatgctggttgagttctatggactggatggatgcttgctgattgtgggtgctttagctttaaatatattagcctgtggcagtctgatgagacccctccaatcttctgattgtcctttgcctaaaaaaatagctccagaagatctaccagataaatactccatttacaatgaaaaaggaaagaatctggaagaaaacataaacattcttgacaagagctacagtagtgaggaaaaatgcaggatcacgttagccaatggtgactggaaacaagacagcctacttcataaaaaccccacagtgacacacacaaaagagcctgaaacgtacaaaaagaaagttgcagaacagacatatttttgcaaacagcttgccaagaggaagtggcagttatataaaaactactgtggtgaaactgtggctctttttaaaaacaaagtattttcagcccttttcattgctatcttactctttgacatcggagggtttccaccttcattacttatggaagatgtagcaagaagttcaaacgtgaaagaagaagagtttattatgccacttatttccattataggcattatgacagcagttggtaaactgcttttagggatactggctgacttcaagtggattaataccttgtatctttatgttgctaccttaatcatcatgggcctagccttgtgtgcaattccatttgccaaaagctatgtcacattggcgttgctttctgggatcctagggtttcttactggtaattggtccatctttccatatgtgaccacgaagactgtgggaattgaaaaattagcccatgcctatgggatattaatgttctttgctggacttggaaatagcctaggaccacccatcgttggttggttttatgactggacccagacctatgatattgcattttattttagtggcttctgcgtcctgctgggaggttttattctgctgctggcagccttgccctcttgggatacatgcaacaagcaactccccaagccagctccaacaactttcttgtacaaagttgcctctaatgtttagaagaatattggaagacactatttttgctattttataccatatagcaacgatattttaacagattctcaagcaaattttctagagtcaagactattttctcatagcaaaatttcacaatgactgactctgaatgaattatttttttttatatatcctattttttatgtagtgtatgcgtagcctctatctcgtatttttttctatttctcctccccacaccatcaatgggactattctgttttgctgttattcactagttcttaacattgtaaaaagtttgaccagcctcagaaggctttctctgtgtaaagaagtataatttctctgccgactccatttaatccactgcaaggcacctagagagactgctcctattttaaaagtgatgcaagcatcatgataagatatgtgtgaagcccactaggaaataaatcattctcttctctatgtttgacttgctagtaaacagaagacttcaagccagccaggaaattaaagtggcgactaaaacagccttaagaattgcagtggagcaaattggtcattttttaaaaaaatatattttaacctacagtcaccagttttcattattctatttacctcactgaagtactcgcatgttgtttggtacccactgagcaactgtttcagttcctaaggtatttgctgagatgtgggtgaactccaaatggagaagtagtcactgtagactttcttcatggttgaccactccaaccttgctcacttttgcttcttggccatccactcagctgatgtttcctgggaagtgctaattttacctgtttccaaattggaaacacatttctcaatcattccgttctggcaaatgggaaacatccatttgctttgggcacagtggggatgggctgcaagttcttgcatatcctcccagtgaagcatttatttgctactatcagattttaccactatcaaatataattcaagggcagaattaaacgtgagtgtgtgtgtgtgtgtgtgtgtgtgctatgcatgctctaagtctgcatgggatatgggaatggaaaagggcaataagaaattaatacccttatgcagttgcatttaaccttaagaaaaatgtccttgggataaactccaatgtttaatacattgattttttttctaaagaaatgggttttaaactttggtatgcatcagaattccctatagatctttttgaaaatataggtacctgggtatcacacatagaacttttaattctgctggtgtaggctgttgcccaaacatctataattttactgagctcttcaagtgattctgataacacagcctggattgagaatttttataagattggcaatggaaaaacatttattcttttaaataataatttttttaaaacccaagaggtcaggggattttataaaccaatagccaagtgttctttaaataggaggcacccttcccattgtgccaaaatcatcttttcatttattttgaaatttgtatgattattttatacttgtatgttgcctttcttcgaaggcgcctgaagcactttataaacacaaatcctcacaatacctctgtgaggtaggtaaatagtacttttctatgtagtaaacctggaatatggagaatttcataacagttcattctacttaataatgcaataatggagctccaagttgtcttggacttctacaccacactcagacttctggaaagttttctgtacctcattctttagtccctgtcaaggttagtaaataaaataagtgacataaaaaaaaaaaaaactaaactacttgttgtgttgaaagttcctttttgccagttatgttcaggaaacccaataacctgaaaaagtttgactttgatgtgacatcttcatattcatcaatgctgataattgtccaaaggcatcttcactatgtctgctaaataacatccaatgtgggcgttatctgttgtctaggggatgaattttaagttacaataaaatatttttctttgttttgca

SEQ ID NO: 103 - DNA, 536 bp, Homo sapiens
ctgggagacacgtcagggagaggtagctgtggtcactgccttgtacaacagccaaaagcccaaagcaggagggaccctggccttctcccagcacacaacgagtgggagctctgtgtgctggccggcatttcctgtcacgttcaataggacacgttcactcttcatacttcttcaattctaaatttcagaagtaatttgtcactttagaggagggcgtcattaataaccattatttagaactgtcgagtcttcctctctgtgagtgtctgagttaagcatccccaaaattggccttgttggtggcaagcagtgcccccacactgacagattgagactaccccacccccaccgacgccctcacaacccagttcttccccgtctgcctttaatcaccgcgaggggggcgacagggaatggctacggcatgtcctcctggaattcattagcgttattaccaaagaccgtgttgtaaattgagattttttttaactgctaggaaaaaatttctccttaactatttcattttattgtactta

SEQ ID NO: 104 - DNA, 862 bp, Homo sapiens
cctgcctcgacaaaattaaaaaaataagtattgttgctccccttttggagatgagtcaaaaagattaaacaactagccccaagtcatggagataattaaaaaagattaaacaactagccccaagtcatggagataattaaaaaagattaaacaactagccccaagtcatggagataaaaaggtcagaatttcttttttagaaacggggtcttactctgttgcccagtctggagtgcaatggcacagtcatggctcaatgtaacctcagactcctgggctcaagcggtcctcccacgtgagcctcccaagactacaggtgcacaccatcatacacgggacagggtctcgctatattgctcagactggaaagttcagatttttaaatcaggtcttgggactcccgattctgtttttccacagagtcaccatctatcctgacaatgctccatttcatgctgtttttcctcaccttcaatactgctcccccatccccccacctctaggtgtgaaggttaccaggagagacctgagctcgctggctctgactccaaggtggcctcagtggaaagtttcaaaaggcaaccggtttggtttcactggcagggcagcggcaggcgtttgggttctggaggcccaggaatgtagaagcctccagctaacagactccacgcgcctatcctcccaaacgctctcggagataagctcccagctccctccccttttccaccttcatgcacttcctgctgtattctgtccattccagcactggccctttctgtgggtgggtgggcagaggatacaatttcctgcatgactacttgctcatgattcatacttctaaatgaaagtacaactgatataa

SEQ ID NO: 105 - DNA, 1072 bp, Homo sapiens
aaaatgtacttagaaattttaaaagcacaaaacaaacgcattctctccccatcctcctatctccagctcttagagactggagctcagcacctaagctgttaatgaatggggacagctttcatctccactggaaaaaagcctgctctctcacttggggtccctctcccccttccacttgcattcaatcagcacccatgcaaccatcctccctgctctgagctctgtgagcccctgaaaatagagaaattgggtgtttgtggagcaaaatatagctaagtaatttttcctgctcctttgaggccatgttctttcatggtgagggaggggcagagaaaatagaggctcacaaatcccttttcctgtgactcccacaacttaggccaggggccttcttgagcctcataatgtgtgtgtgtagataggggaaaggaggtccacttccagaattttccctgtgttcttattcctcacttatgctaccgttggctcagctggcccgaaccaagatccatagccaggtttccatcactgatgagctccccaaaacagggtgaccttcccctcctcgtggggtaaggaaagctctcatatcattggacttcaggcaggaagggtcagttggaaagaaacctttgacgtgagcctcttgatgtctccatggcctctgtgcctccatgctggcccaggccttctgtgcttatgcccaggaagcatgtggccagtgaatgaatgcacccaggatgcctccttcttttccatgggagcccagaagatgccacttggagctcagcgtcctggtgtctagaaaagtttctggtgccagcagtgctgctccatttggtacagcaggtgccaagcctctcaatggaggctctttggacttctatgaaaaattattaatgagcttccagactttcatatctggcatttattctccaatggatacctgaggaaaaacctttttcttcatcaaatagaacttgaggagaaatcaaaaagacaacttcaggaggcaacagatgggaagtgcctgcctttaaacaaaacaaaacataaacaggctttatgcctt

SEQ ID NO: 106 - DNA, 856 bp, Homo sapiens
cagtttcatgtgcttaagcaactttgcttcaggtcacaccctacgggacacccacggcagcctgccgcctactaatcatagagcccttcgtgttccttttttgtctttttcttaaccaacaatgggtcatttagcaggacatttatttcagtcctaagttgtattatccctggtaatttgcatataccattattaaagtgtggcagtcttttgtaattattgtcttaatctagtgaaaaataatatatctgtatatctggagagaaggctgttctctggatgcagctgagcacttgcatgcactcgatgaacgggaataggactgcatgaggctgacctggatttgacaaccgcaccaggacaaggccgcgtgctgccctgaacagtggcccttgtgctaaatacgaatcctctctctcccacaggcatagcccgtcacctgcgtctggtttttgctcctcattttcttcaattttcactctatttatagttgagaaccttccatttccccctggttgaaatacattagttgctatggaaactgcgatccccccggtgtggatggagctgaatgacacctacaattgcagagcacggttggcgttgccagggctgggaaatgggcgtcgtggctggagagggcactgaagggcacagatgagaataatgacagcacacagcacgaccgtcaggaaccgacgcagcaccactgggtcagaagttgtggaagaagccatgggtaacagaagccccccatgccctacaccacacagaggggcgggtcccatcagaggcctaacccctggagggctctcattttcaaaacataaaaaatggagctatagctggtacttgc

SEQ ID NO: 107 - DNA, 1155 bp, Homo sapiens
gagtcctaattagggaaaaggagtcaggctggtgggaccaaggaaaagcaaagagaaagcacataagctgtaagtctgcctttcttcatggtccaggacacataatcctcctgcgtaaataagtcacaatcttcctgcgcccagctatcatcagaccctcggctgatagaaaaatgcaaattagctcactgcaaccttggcattatcagtactgcacatagctctctccagaaaacagcacgaacaccatcctataaaatccacagcaagcctttgtctcctcacagtcagctcccttctttctgacttgcccactgctttcttgcaacgcaatttcatacttgtgattcttatgcctcagccatccaagtagctgggattacagcatgcgctaccacacctggcttttttattattattatttttggagagatgacatcttgctatgttgtccaggccactctcaaactcctggaataaagggatcctcccacattggcctcccaaagtgctgggattatagataggtgtgaaccatcatacccagctttattttatttttttgtagagataggggtctcgttcacttgcccaggctggaatgtccggttttactttcctgttttttcttggtggcagataccatttgtttgctttcagatataacattcccctaagcacttcttgtaggccgagtctagtggtgatgcattccctcagtttttacttgtctgggaaacactttatttctccttttcttcttcagggagttttaatttttcttaaacatgtggtcactctctagaggtgggacacccccaccccatttttggcttagatcttctcgtgtgtcgacttgtgtccccctagaaggagtgttgaagtcctaacccacagtacctgtgattgtgatctttttttgagatagggtgtgatttctaaaaaattatatgtgattagttaaaatgagttcacagtggattagggtgggttcacatataataagactgatattcttacaagaagtggagaagagacccagaggggagaaagccatgtgaagacagaggtggaagctggtgtagctgtcaagccacacatacactgtattagtttcctgtggttcctgtcaaaggtaccacaaactggtgagtt

SEQ ID NO: 108 - DNA, 3344 bp, Homo sapiens
ggaccggctccggaccgcgcagttagcgccgcctggcctgggccggacccggtcagggttctcaagctgtcgtccctatggggctgtgttttccttgtcccggggagtccgcgcctcccacgccggacctggaagagaaaagagcaaagcttgcagaggctgcagagagaagacaaaaagaggctgcatctcggggaattttagatgttcaatctgtgcaagaaaagagaaagaaaaaggaaaaaatagaaaaacaaattgctacatccgggcccccaccagaaggtggacttaggtggacagtttcataaagcataacatgagtagaagaatctactgccaataactgtttattatctgcaatcaagtgggcttcatcaatttaatttcttctctttgagtaaatgaagattcagactttgtaatattattgcccttaagtgcaatgctaaaaaaacgttgattttcaagcttagagaatggctagacttttcattaaatactgattttcctacatttgctcttctgcagttagtgggtgatttgctatttttcttagtagttaaaaaatggaactaaatagtgaatatacatacactgcatgtaaacattctgcatatacctctaagattaaaattcgcagttgtcttttcatcctttataaaatgatctaactacttatatttgtgctgcatcgcgttacatctgtttttatttcactatgaagatgtttgattaaacttatggacttagtgcctttaaactgatcatcagggagaatcttgaaaaaatcatttgaagggctgatgtgaaggagcactgtaaatttttataacttagtaatgagtattcttaggcagatgtaaaattttttccaatttatttttatttatgtagcttataaaattaacataccctgttttactttatgataaaggattttttgtttgctgaatttaaaattatatattagtgataccatcagagggcagtgatgttctattgtatattaaattcagctctgtaaggatctttgtagtaattgaatgagttaaactaataatctggatgggttataatgagtagtaatatatttgtccatatttcataagtagtgttaatcttgtgtacttattagagaacgatcataagatttatacagatgtgaaactgcgaaggcaagtatgaatgtatgaaaaaaacatgtaggtactgtacttacaaaaggtctacttcagatataaaaatattaggtaattctatacaatgcatagtcataaaccttaacatttttgttcattagaaacatgaattttatagcattttttgtttctcctatataatacactgaaataaaagaatttgtgttagctattaaggctgatagctcttttaaatggcaaggccacatgttgagccctaaattaaaatttgcagatattaagtgctaatagaaattttaagttaaatcgaccaagttcacttgctttacacaaaggaaactgagccactatcttcatctacccctccaacaaaaattatgttatactgcagtgtattgtacatgttaatttttaaaagtttgaactattatataatacaggtctcttgacttctcatggaaaaattattttttctattatggtgtgaaatattgtgtgaatatctaggcaaaacataacaatttggctcaattttcttctttagaggattcgtgctgtttttgttcataaagggtagtgaaatcattgaactatattttagaatgaaaatttttgattttattaaaatgattttttcaaggcagaaagtaaaaggaatgattgatagcggagtgcatatagagctagagcatatcatccttgaactctgcaaatcctttcttccattttaatatagcaagaacaattttgtctttactacatcttaaagaattagaacttgggttggtgtaagtgacttacttccagggaatcatgccctatttctaccagcaggtcatacccaaatgtcacactatctattgttaaccatgaatgatattcagatctattacttttcgtgaaaagtggaacatgttacttccaaccatggcctgtcaccgtgagtgtgatcagctttctccaaaaccacatgggtcgcaggagctaaggggtggtacccaaatgttaggaacagtgttaggaaagggcaagggaaaagaagtgactggatgtcttatgagaaaccggtaaatgactaaaaaaaaaagcaaatgactaaaaacatgactaaaaaattatatatatatataatatatatattatatatgtgtgtatatatatacacataatatctgcaaattctaatttatatatgtgtgtgtatatacacacacacacatgcacatacacacatacgtccagacatctccctcataaaataaccatcagtttctatgaaaaccttaagtggaagccaatttcccatagtaaataatttaggagaaaattataatgcttaaaatgttgctcaaacccctgacctattactaaactataattggaacagtaaaatgcatatatgtaactatcatatcatgatttaaaattgcttaaaccattgctgcttaatactaatcaaacttaacggctgctaacaaaagttgtgaattattacacggcctctttgtaacgtgctgcatgttttttaaaacatctctgtgtttctgtttgttccactgctggtatttggaatgtaatttaacagttctcacacatggtttggttataaattctgtattgccttttagggatataaatatacatttttttctatgtaaaaattagctttagctgtctctttaacaaaattttatctttactacatcctaaatacttagaacctgagttggtggttagggaaacctcaggaacattttaatcacattgggattcagaagagcaacagaaccaaaggttgtttggtgtgttcatacaatccctggatttataggtggattttctataaaggaaaaatgatgtaattagtatcctgttttttcctaaagaaataatactatcataaaaattctgtctatcctttgtaccccaggaaaatggacatgaactttgaattttccctttctccaaatgtttgactttttattttcactgataagcattatgctatgttcttagaagacaaaagcagctcttgccagttttgaataatttctgcatgaatagaccagtaagaggtaagtagccatgactgcctatatgtgttgagacataaggtatatttctttaacatctccaagcaagcatttcaaattctcttaactactaaacatgctctaagct

SEQ ID NO: 109 - protein, 490 aa, Homo sapiens
MDGNDNVTLLFAPLLRDNYTLAPNASSLGPGTNLALAPASSAGPALGSASGRYRASASARPHSDPGAHDQRPRGRRGEPRPFPVPSALGAPRAPVLGHAAEPRAERVRGRRLCITMLGLGCTVDVNHFGAHVRRPVAALLAALPVRPPAAAGLPAGPRLQAGRGGRRGLLLCGCCPGGNLSNLMSLLVDGDMNLRRAALLALSSDVGSAQTSTPGLAVSPFHLYSTYKKKVSWLFDSKLVLISAHSLFCSIIMTISSTLLALVLMPLCLWIYSWAWINTPIVQLLPLGTVTLTLCSTLIPIGLGVFIRYKYSRVADYIVKVSLWSLLVTLVVLFIMTGTMLGPELLASIPAAVYVIAIFMPLAAYASGYGLATLFHLPPNCKRTVCLETGSQNVQLCTAILKLAFPPQFIGSMYMFPLLYALFQSAEAGIFVLIYKMYGSEMLHKRDPLDEDEDTDISYKKLKEEEMADTSYGTVKAENIIMMETAQTSL

SEQ ID NO: 110 - protein, 153 aa, Homo sapiens
MMKEFSSTAQGNTEVIHTGTLQRHESHHIRDFCFQEIEKDIHNFEFQWQEEERNGHEAPMTEIKELTGSTDRHDQRHAGNKPIKDQLGSSFHSHLPELHIFQPEWKIGNQVEKSIINASLILTSQRISCSPKTRISNNYGNNSLHSSLPIQKL

SEQ ID NO: 111 - protein, 385 aa, Homo sapiens
MSGSSGTPYLGSKISLISKAQIRYEGILYTIDTDNSTVALAKVRSFGTEDRPTDRPAPPREEIYEYIIFRGSDIKDITVCEPPKAQHTLPQDPAIVQSSLGSASASPFQPHVPYSPFRGMAPYGPLAASSLLSQQYAASLGLGAGFPSIPVGKSPMVEQAVQTGSADNLNAKKLLPGKGTTGTQLNGRQAQPSSKTASDVVQPAAVQAQGQVNDENRRPQRRRSGNRRTRNRSRGQNRPTNVKENTIKFEGDFDFESANAQFNREELDKEFKKKLNFKDDKAEKGEEKDLAVVTQSAEAPAEEDLLGPNCYYDKSKSFFDNISSELKTSSRRTTWAEERKLNTETFGVSGRFLRGRSSRGGFRGGRGNGTTRRNPTSHRAGTGRV

SEQ ID NO: 112 - protein, 568 aa, Homo sapiens
MALPKDAIPSLSECQCGICMEILVEPVTLPCNHTLCKPCFQSTVEKASLCCPFCRRVSSWTRYHTRRNSLVNVELWTIIQKHYPRECKLRASGQESEEVGDDYQPVRLLSKPGELRREYEEEISKVAAERRASEEEENKASEEYIQRLLAEEEEEEKRQAEKRRRAMEEQLKSDEELARKLSINNFCEGSISASPLNSRKSDPVTPKSEKKSKNKQRNTGDIQKYLTPKSQFGSASHSEAVQEVRKDSVSKDIDSSDRKSPTGQDTEIEDMPTLSPQISLGVGEQGADSSIESPMPWLCACGAEWYHEGNVKTRPSNHGKELCVLSHERPKTRVPYSKETAVMPCGRTESGCAPTSGVTQTNGNNTGETENEESCLLISKEISKRKNQESSFEAVKDQCFSAKRRKVSPESSPDQEETEINFTQKLIDLEHLLFERHKQEEQDRLLALQLQKEVDKEQMVPNRQKGSPDEYHLRATSSPPDKVLNGQRKNPKDGNFKRQTHTKHPTPERGSRDKNRQVSLKMQLKQSVNRRKMPNSTRDHCKVSKSAHSLQPSISQKSVFQMFQRCTK

SEQ ID NO: 113 - protein, 645 aa, Homo sapiens
MWIQVRTIDGSKTCTIEDVSRKATIEELRERVWALFDVRPECQRLFYRGKQLENGYTLFDYDVGLNDIIQLLVRPDPDHLPGTSTQIEAKPCSNSPPKVKKAPRVGPSNQPSTSARARLIDPGFGIYKVNELVDARDVGLGAWFEAHIHSVTRASDGQSRGKTPLKNGSSCKRTNGNIKHKSKENTNKLDSVPSTSNSDCVAADEDVIYHIQYDEYPESGTLEMNVKDLRPRARTILKWNELNVGDVVMVNYNVESPGQRGFWFDAEITTLKTISRTKKELRVKIFLGGSEGTLNDCKIISVDEIFKIERPGAHPLSFADGKFLRRNDPECDLCGGDPEKKCHSCSCRVCGGKHEPNMQLLCDECNVAYHIYCLNPPLDKVPEEEYWYCPSCKTDSSEVVKAGERLKMSKKKAKMPSASTESRRDWGRGMACVGRTRECTIVPSNHYGPIPGIPVGSTWRFRVQVSEAGVHRPHVGGIHGRSNDGAYSLVLAGGFADEVDRGDEFTYTGSGGKNLAGNKRIGAPSADQTLTNMNRALALNCDAPLDDKIGAESRNWRAGKPVRVIRSFKGRKISKYAPEEGNRYDGIYKVVKYWPEISSSHGFLVWRYLLRRDDVEPAPWTSEGIERSRRLCLRGLCLGKVGPVN

SEQ ID NO: 114 - protein, 284 aa, Homo sapiens
MIKSSSLTRACPPHPRQQGGEWGNKITTKSLGVSHSPSPGTLSETLQSPRNSLREAGRRPAIWTKLRYADADRAALRGEDPGGASSAGSSSQKTDDPERVAGTDCQAFGGGTGSGRLGSAFKMASPQGGQIAIAMRLRNQLQSVYKMDPLRNEVQGRQGYCCGRPAEEVRVKIKDLNEHIVCCLCAGYFVDATTITECLHTFCKSCIVKYLQTSKYCPMCNIKIHETQPLLNLKLDRVMQDIVYKLVPGLQDSEEKRIREFYQSRGLDRVTQPTGEGMSLAAGQ

SEQ ID NO: 115 - protein, 195 aa, Homo sapiens
MVGGGGVGGGLLENANPLIYQRSGERPVTAGEEDEQVPDSIDAREIFDLIRSINDPEHPLTLEELNVVEQVRVQVSHFRGERVVPESQKGFCAAGAGVLYAHEHRRVSDPESTVAVAFTPTIPHCSMATLIGLSIKVKLLRSLPQRFKMDVHITPGTHASEHAVNKQLADKERVAAALENTHLLEVVNQCLSARS

SEQ ID NO: 116 - protein, 812 aa, Homo sapiens
MEAFQELRKPSARLECDHCSFRGTDYENVQIHMGTIHPEFCDEMDAGGLGKMIFYQKSAKLFHCHKCFFTSKMYSNVYYHITSKHASPDKWNDKPKNQLNKETDPVKSPPLPEHQKIPCNSAEPKSIPALSMETQKLGSVLSPESPKPTPLTPLEPQKPGSVVSPELQTPLPSPEPSKPASVSSPEPPKSVPVCESQKLAPVPSPEPQKPAPVSPESVKATLSNPKPQKQSHFPETLGPPSASSPESPVLAASPEPWGPSPAASPESRKSARTTSPEPRKPSPSESPEPWKPFPAVSPEPRRPAPAVSPGSWKPGPPGSPRPWKSNPSASSGPWKPAKPAPSVSPGPWKPIPSVSPGPWKPTPSVSSASWKSSSVSPSSWKSPPASPESWKSGPPELRKTAPTLSPEHWKAVPPVSPELRKPGPPLSPEIRSPAGSPELRKPSGSPDLWKLSPDQRKTSPASLDFPESQKSSRGGSPDLWKSSFFIEPQKPVFPETRKPGPSGPSESPKAASDIWKPVLSIDTEPRKPALFPEPAKTAPPASPEARKRALFPEPRKHALFPELPKSALFSESQKAVELGDELQIDAIDDQKCDILVQEELLASPKKLLEDTLFPSSKKLKKDNQESSDAELSSSEYIKTDLDAMDIKGQESSSDQEQVDVESIDFSKENKMDMTSPEQSRNVLQFTEEKEAFISEEEIAKYMKRGKGKYYCKICCCRAMKKGAVLHHLVNKHNVHSPYKCTICGKAFLLESLLKNHVAAHGQSLLKCPRCNFESNFPRGFKKHLTHCQSRHNEEANKKLMEALEPPLEEQQI

SEQ ID NO: 117 - protein, 672 aa, Homo sapiens
MPGMVLFGPALAIASDDLVFPGFFELVVRVLWWIGILTLYLMHRGKLDCAGGALLSSYLIVLMILLAVVICTVSAIMCVSMRGTICNPGPRKSMSKLLYIRLALFFPEMVWASLGAAWVADGVQCDRTVVNGIIATVVVSWIIIAATVVSIIIVFDPLGGKMAPYSSAGPSHLDSHDSSQLLNGLKTAATSVWETRIKLLCCCIGKDDHTRVAFSSTAELFSTYFSDTDLVPSDIAAGLALLHQQQDNIRNNQEPAQVVCHAPGSSQEADLDAELENCHHYMQFAAAAYGWPLYIYRNPLTGLCRIGGDCCRSRTTDYDLVGGDQLNCHFGSILHTTGLQYRDFIHVSFHDKVYELPFLVALDHRKESVVVAVRGTMSLQDVLTDLSAESEVLDVECEVQDRLAHKGISQAARYVYQRLINDGILSQAFSIAPEYRLVIVGHSLGGGAAALLATMLRAAYPQVRCYAFSPPRGLWSKALQEYSQSFIVSLVLGKDVIPRLSVTNLEDLKRRILRVVAHCNKPKYKILLHGLWYELFGGNPNNLPTELDGGDQEVLTQPLLGEQSLLTRWSPAYSFSSDSPLDSSPKYPPLYPPGRIIHLQEEGASGRFGCCSAAHYSAKWSHEAEFSKILIGPKMLTDHMPDILMRALDSVVSDRAACVSCPAQGVSSVDVA

SEQ ID NO: 118 - protein, 510 aa, Homo sapiens
MELKKSPDGGWGWVIVFVSFLMPFIAQGQGNLINSPTSPLAIGLIYILKKEVEHHYKKGEMKASLFIKSPYAVQNIRKTAAVGVLYIEWLDAFGEGKGKTAWVGSLASGVGLLASLGCGLLYTATVTITCQYFDDRRGLALGLISTGSSVGLFIYAALQRMLVEFYGLDGCLLIVGALALNILACGSLMRPLQSSDCPLPKKIAPEDLPDKYSIYNEKGKNLEENINILDKSYSSEEKCRITLANGDWKQDSLLHKNPTVTHTKEPETYKKKVAEQTYFCKQLAKRKWQLYKNYCGETVALFKNKVFSALFIAILLFDIGGFPPSLLMEDVARSSNVKEEEFIMPLISIIGIMTAVGKLLLGILADFKWINTLYLYVATLIIMGLALCAIPFAKSYVTLALLSGILGFLTGNWSIFPYVTTKTVGIEKLAHAYGILMFFAGLGNSLGPPIVGWFYDWTQTYDIAFYFSGFCVLLGGFILLLAALPSWDTCNKQLPKPAPTTFLYKVASNV

SEQ ID NO: 119 - DNA, 47 bp, unknown
  Oligo d(t) oligo with Not I site.
aagcagtggtaacaacgcagagtgcggccgatttttttttttttttr

SEQ ID NO: 120 - DNA, 30 bp, unknown
  Combination DNA with three ribonucleotides at the 3′ end.
aagcagtggtaacaacgcagagtcgacggg

SEQ ID NO: 121 - DNA, 27 bp, unknown
  Oligonucleotide directed to the minus strand of the pSport vector.
aagcagtggtaacaacgcagagtcgac

SEQ ID NO: 122 - protein, 8 aa, bacteriophage T7
DYKDDDDK

SEQ ID NO: 123 - DNA, 733 bp, homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 124 - protein, 29 aa, Artificial
  Synthetic peptide sequence.
PKKKRKVAAVALLPAVLLALLAPKKKRKV

SEQ ID NO: 125 - DNA, 2503 bp, Homo sapiens
ctggccccgagcagctgaagcctggggtcagcaggcgctgcgggcgcagctccggtgcaagcgaggacacgacacatgcagtggcttctggactgcgcgatgactggacgcaagtaacttctaggtctgcagacaagaggaagagaagatgaaggaagactgtctgccgagttctcacgtgcccatcagtgacagcaagtccattcagaagtcggagctcttaggcctgctgaaaacctacaactgctaccatgagggcaagagcttccagctgagacaccgtgaggaagaagggactctgatcatcgaggggctcctcaacattgcctgggggctgaggcggcccatccggctgcagatgcaggatgaccgggagcaggtgcacctcccctccacctcatggatgcccagacggcctagctgccctctaaaggagccatcgccccagaacgggaacatcacagcccaggggccaagcattcagccagtgcacaaggctgagagttccacagacagctcggggcccctggaggaggcagaggaggccccccagctgatgcggaccaagagcgacgccagttgcatgagccagaggaggcccaagtgccgcgcccccggtgaggcccagcgcatccggcgacaccggttctctatcaacggccacttctacaatcataagacctccgtgtttactccagcctatggatccgtgaccaatgtgagggtcaacagcaccatgacaaccctgcaggtgctcaccctgctgctgaacaaatttagggtggaagatggccccagtgagttcgcactctacatcgttcacgagtctggggagcggacaaaattaaaagactgcgagtacccgctgatttccagaatcctgcatgggccatgtgagaagatcgccaggatcttcctgatggaagctgacttgggcgtggaagtcccccatgaagtcgctcagtacattaagtttgaaatgccggtgctggacagttttgttgaaaaattaaaagaagaggaagaaagagaaataatcaaactgaccatgaagttccaagccctgcgtctgacgatgctgcagcgcctggagcagctggtggaggccaagtaactggccaacacctgcctcttccaaagtccccagcagtggcaggtgtacactgagccctggttgctggccccggccggtcacattgactgatggccaccgcctgacgaatcgagtgcctgtgtgtctacctctctgaagcctgagcaccatgattcccacagccagctcttggctccaagatgagcacccacaggaagccgacccaggcctgaggggccaggaacttgctgggtcagatctgtgtggccagccctgtccacaccatgcctctcctgcactggagagcagtgctggcccagcccctgcggcttaggcttcatctgcttgcacattgcctgtcccagagcccctgtgggtccacaagcccctgtcctcttccttcatatgagattcttgtctgccctcatatcacgctgccccacaggaatgctgctgggaaaagcagggcctgccagcaggtatgagatctagcctgctttcagccatcaccttgccacagtgtccccggcttctaagcctccaatatcaccctgtgagcctcgcacagctcagccccaacacagaggtgagaccaggaataaggccacaagtatctcactttctctgcagaaatcaatctttacttcatcagagagacctaaagcgattcttacaaggagcttgctgcaagaaacacggtcattcaatcacattgaggagggtccacatggcattgagagggtgctgcccgctcaatgcccagcagcagctctggaaggcagtgctcagccccatcaccactgtcccgtggatgcctgtgtacctcttgccttttctgggcttgcgtttctctcctctagtgggtggggatgactttcaatgactttcaatacttcccctgaaggaagaatgataaggagaaatgtctgttttgaggaaagggctttgaattccccagatactgaacaatttgtgtttgtgactgatggagaatttcaggaatgaatgagaaagcctttgcgaaactatgcaacagtttacatcagtcatgtgaagtatttgtctaaaacagagcaaactgaagaccaaattattctcctgttgaggtccgtggatggcagatttaaagggaagaaccacaaaggcttgcaaagataggagaggctccatctctaatgcatgtagaagctccttacgggtgcccatcaagagcatagcttggaagccaccatgctgtgcggaactgcgtcagggcaaatgtcacagcaggatttccccaacccagctccatcatcacagacacagagagctgcaggggaggcctgcccactgttttgtcgactctgccctcctctggcagcatagatccttaggtgctcaataaaggtgtgctgtattgaactgaagaag

SEQ ID NO: 126 - protein, 321 aa, Homo sapiens
MKEDCLPSSHVPISDSKSIQKSELLGLLKTYNCYHEGKSFQLRHREEEGTLIIEGLLNIAWGLRRPIRLQMQDDREQVHLPSTSWMPRRPSCPLKEPSPQNGNITAKGPSIQPVHKAESSTDSSGPLEEAEEAPQLMRTKSDASCMSQRRPKCRAPGEAQRIRRHRFSINGHFYNHKTSVFTPAYGSVTNVRVNSTMTTLQVLTLLLNKFRVEDGPSEFALYIVHESGERTKLKDCEYPLISRILHGPCEKIARIFLMEADLGVEVPHEVAQYIKFEMPVLDSFVEKLKEEEEREIIKLTMKFQALRLTMLQRLEQLVEAK

SEQ ID NO: 127 - DNA, 2076 bp, Homo sapiens
cagtcttgtttcgggttccggctgcgttgggcttgcgtgcggctcgctaagactatggcgtccgggcctcattcgacagctactgctgccgcagccgcctcatcggccgccccaagcgcgggcggctccagctccgggacgacgaccacgacgacgaccacgacgggagggatcctgatcggcgatcgcctgtactcggaagtttcacttaccatcgaccactctctgattccggaggagaggctctcgcccaccccatccatgcaggatgggctcgacctgcccagtgagacggacttacgcatcctgggctgcgagctcatccaggccgccggcattctcctccggctgccgcaggtggcgatggcaacggggcaggtgttgtttcatcgttttttctactccaaatctttcgtcaaacacagtttcgagattgttgctatggcttgtattaatcttgcatcaaaaatcgaagaagcacctagaagaataagagatgtgattaatgtattccaccacctccgccagttaagaggaaaaaggactccaagccccctgatccttgatcagaactacattaacaccaaaaatcaagttatcaaagcagagaggagggtgctaaaggagttgggattttgtgttcatgtcaagcatcctcataagatcattgttatgtatttacaagtcttagaatgtgaacgtaatcaaaccctggttcaaactgcctggaattacatgaatgacagtcttcgaaccaatgtgtttgttcgatttcaaccagagactatagcatgtgcttgcatctaccttgcagctagagcacttcagattccgttgccaactcgtccccattggtttcttctttttggtactacagaagaggaaatccaggaaatctgcatagaaacacttaggctttataccagaaaaaagccaaactatgaattactggaaaaagaagtagaaaaaagaaaagtagccttacaagaagccaaattaaaagcaaagggattgaatccggatggaactccagccctttcaaccctgggtggattttctccagcctccaagccatcatcaccaagagaagtaaaagctgaagagaaatcaccaatctccattaatgtgaagacagtcaaaaaagaacctgaggatagacaacaggcttccaaaagcccttacaatggtgtaagaaaagacagcaagagaagtagaaatagcagaagtgcaagtcgatcgaggtcaagaacacgatcacgttctagatcacatactccaagaagacactataataataggcggagtcgatctggaacatacagctcgagatcaagaagcaggtcccgcagtcacagtgaaagccctcgaagacatcataatcatggttctcctcaccttaaggccaagcataccagagatgatttaaaaagttcaaacagacatggtcataaaaggaaaaaatctcgttctcgatctcagagcaagtctcgggatcactcagatgcagccaagaaacacaggcatgaaaggggacatcatagggacaggcgtgaacgatctcgctcctttgagaggtcccataaaagcaagcaccatggtggcagtcgctcaggacatggcaggcacaggcgctgactttgtcttcctttgagcctgcatcagttcttggttttgcctatctaccagtgtgatgtatggactcaatcaaaaacattaaacgcaaaactgattaggatttgatttcttgaaaccctctaggtctctagaacactgaggacagtttcttttgaaaagaactatgttaatttttttgcacattaaaatgccctagcagtatctaattaaaaaccatggtcaggttcaattgtactttattatagttgtgtattgtttattgctataagaactggagcgtgaattctgtaaaaatgtatcttatttttatacagataaaattgcagacactgttctatttaagtggttatttgtttaaatgatggtgaatactttcttaacactggtttgtctgcatgtgtaaagatttttacaaggaaataaaatacaaatcttgttttt

SEQ ID NO: 128 - protein, 526 aa, Homo sapiens
MASGPHSTATAAAAASSAAPSAGGSSSGTTTTTTTTTGGILIGDRLYSEVSLTIDHSLIPEERLSPTPSMQDGLDLPSETDLRILGCELIQAAGILLRLPQVAMATGQVLFHRFFYSKSFVKHSFEIVAMACINLASKIEEAPRRIRDVINVFHHLRQLRGKRTPSPLILDQNYINTKNQVIKAERRVLKELGFCVHVKHPHKIIVMYLQVLECERNQTLVQTAWNYMNDSLRTNVFVRFQPETIACACIYLAARALQIPLPTRPHWFLLFGTTEEEIQEICIETLRLYTRKKPNYELLEKEVEKRKVALQEAKLKAKGLNPDGTPALSTLGGFSPASKPSSPREVKAEEKSPISINVKTVKKEPEDRQQASKSPYNGVRKDSKRSRNSRSASRSRSRTRSRSRSHTPRRHYNNRRSRSGTYSSRSRSRSRSHSESPRRHHNHGSPHLKAKHTRDDLKSSNRHGHKRKKSRSRSQSKSRDHSDAAKKHRHERGHHRDRRERSRSFERSHKSKHHGGSRSGHGRHRR

SEQ ID NO: 129 - protein, 326 aa, Homo sapiens
MDYSHQTSLVPCGQDKYISKNELLLHLKTYNLYYEGQNLQLRHREEEDEFIVEGLLNISWGLRRPIRLQMQDDNERIRPPPSSSSWHSGCNLGAQGTTLKPLTVPKVQISEVDAPPEGDQMPSSTDSRGLKPLQEDTPQLMRTRSDVGVRRRGNVRTPSDQRRIRRHRFSINGHFYNHKTSVFTPAYGSVTNVRINSTMTTPQVLKLLLNKFKIENSAEEFALYVVHTSGEKQKLKATDYPLIARILQGPCEQISKVFLMEKDQVEEVTYDVAQYIKFEMPVLKSFIQKLQEEEDREVKKLMRKYTVLRLMIRQRLEEIAETPATI

SEQ ID NO: 130 - protein, 328 aa, Mus musculus
MTAMDHQFPSWIVVNESTSISREQLNYLLETYNVFYENQKNLHILYGQTEDGQLIVEGMLDIFWGVKRPIQLKIQDEKQISSFDLLNTPETFSSKGRMTRWGEFDDLYRISELDRTHVLASEARHSPEDEEPESPLLYRTMSEAALVRKRMRAPEMYRKDRMGVLSNHRASINGHVYDHETSIFTPTFGSETKVRANSIMRTEEVIKQLLQKFKIENSPRDFALYIIFGTGEQRKLKKTDVPLLQRLLQGPSKSNARIFLMDKDAEEISRDVAPYINFHFSFLESILQRLDEEEKMEIERIMAKFNTERAFILKCLQSKQAAKTETTV

SEQ ID NO: 131 - protein, 826 aa, Drosophila melanogaster
MWKCHKCGKPVYFAERKQSIGYDWHPECLRCEECGKRLNPGQHAEHKSVPYCHVPCYGALFGPQLFGHGTRVESHKSYGVKGAQKPTGAQANGPPLPRDHLESKLKVYNQFYDNKSLEIRSREVNNRLVLEGALRVYWGVQGVIHLKEDDDQRILVRKRNSCRVSKAANESSSDKENEASESLAPPTTTTAEVDQLSTDVSLSESMTFDSCSLNEISELPTTPEDASANTTANSKEQTNGNVCNDDEDTTTTDSSGTLVEAPTASTSCVSSTLPSKLDRLEKLDWDDIDDLLQVERRHNDKDRIYETMPVKLPSSQSSSDSSPSKTSTETTTTTESSSTQSASTNTSSTDDFMTATGSLTANTNTQNTTTVSTTETSLDNFETCDDATLKPIDFEDFKRSVHQDYVNGANSFTEPNEGTLKRNQPIDPSRIHDSLKLYGENSAMSKSFNCEHALRSIDPTLINDTMNLRSSVGSPHSAQRQYALQKSGSATVTSRDQKKPYQQGRQLFEKGINRSKSGPSCFVYSDSDDDDEATLRPQRMATIRRSDIPQRYIQIQMDCYPKENVAAASEGESSRADAPSITSGAAAGDELTQTEDLYTASEGVDGPDGDGSAGLHVTEDGVVLRRPPRTGASAIKRRSGNRRSRTKLKRRCSINGHYYNRETSFFTPPYGSQMSVWVSSMVTTTEVINLVLEKYKVDSSPGNFSLFIVRDNGEQKRLKDDEYPLITRVTLGPHEDVARIFLVDSRKTDEIRQVITLLFNRSLLTFLLHRCSNEVAQFLNLSLPECRAILERYDQELAREVAKIKERYAELRRRIVSRMESLKVHL

SEQ ID NO: 132 - DNA, 3461 bp, Homo sapiens
cggtctgaaagcgacatccgctatctgcttggctatgtcagccagcagggagggcagcgctccacgcccctcatcatcgcagcccgcaatggacacgcaaaggtggtacgcttgctcttagaacattaccgggtgcagactcagcagactggcaccgtccgcttcgacgggtatgtcattgatggtgccactgctctttggtgtgcagctggagcaggacattttgaagttgttaaacttctagtcagccatggagccaacgtgaaccataccacagtaactaattcaacccccctgcgggcagcatgctttgatggcagactggacattgtgaaatacttggttgaaaataatgccaacatcagcattgccaacaaatatgacaacacctgcctaatgattgcggcatataagggacacactgatgtggtcagataccttttagaacaacgtgctgatcccaatgccaaagcacattgtggagccacagcattgcactttgcagctgaagctgggcacatagatattgtgaaagagctgataaaatggcgtgctgctatagtagtgaatggccatgggatgacgccattgaaagtagctgccgaaagctgtaaagctgatgtcgtagaactgttactctctcatgctgattgcgaccgaagaagtcggattgaagctttggaactcttgggtgcctcctttgcaaatgaccgtgagaactatgacatcataaagacataccactatctatatttagccatgttagagaggttccaagatggtgataacattctcgaaaaagaggttcttccaccaatccatgcttatgggaatagaactgaatgtagaaatcctcaggaactggagtccattcggcaagacagagatgctcttcatatggaaggccttatagttcgggaacggattttaggtgctgacaatattgatgtttctcatcccatcatttacagaggagctgtttatgcggataatatggaatttgagcagtgtatcaagttgtggcttcatgccctgcacctcagacaaaaaggtaacaggaacacccacaaggatcttcttcgatttgctcaagttttctcacaaatgatacatttgaatgaaactgtgaaggccccagacatagaatgtgttttgagatgcagtgttttggaaatagaacaaagtatgaacagagtgaaaaatatttcagatgctgatgtccacaatgctatggacaattatgaatgtaatctctatacctttctgtatttagtgtgcatctctaccaaaacacagtgcagcgaagaagatcagtgcaaaattaacaagcagatctacaacctgattcaccttgatcccagaactcgtgaaggtttcaccttgctgcatctggctgtcaattccaatactccagttgatgatttccacaccaatgacgtctgcagctttccaaatgcacttgtcacaaagctcctgctggactgtggtgctgaggtgaatgccgtggacaatgagggaaacagtgcccttcatattatcgttcagtacaacaggcccatcagtgattttttgaccttgcactccatcatcattagcctagttgaagccggagctcacactgacatgacgaataaacagaataagactccgctagacaaaagtacaactggggtatctgaaatactgcttaaaactcaaatgaagatgagtctcaagtgcctggctgcccgagcagttcgggctaatgacattaactaccaagaccagatccccagaactcttgaagagtttgttggatttcattaagtgactggatatgtaaagtcgtttaatgtggtgctaaaaagtaaaggacttttaatcacagacagtagaattatgtgttcataaattctgcttttctttccactacccttcctcccatcccatccttccttagttctgtatttgtttttcttgcctcatggtaattgatttcagacagactttaacaaaaccacattgttttggtgtaactataaggtatttgcatattggttacctatttgtctttcttttttttaaaggaacagatataaaatgttttgtttatgtaacaagggacatttataatttcaagttgataatgttttaaacagctgcttacaaaagtatttctgttaagcctatgtcagcatgttatccatgcagcagttttgaggattttatgaagaaaaagagctaaaaaggaacattaagaggaatgggatatccaggtgttctgcacatgccaaactgctgtagatagtttacactcttccattatttatacggagtgatgcagcacattttagcattcaggaggatttttaaaaaatagctgcagattaatctggaaaatgtgctaatttaataatagttacaaatttataaatttaaatccatttgaaattgttgcattatgctgggtagtatatacaaaatggttatcatcttaaaccaacttttcagagaatcttgatggactctgcctttaggcttgaattcttcaaagtctattttaatgaaatttatctaaattgcagcagtctatttgattcagctcatagacatgtaaaaattatgaatgctgttttcttatgaaacaaattgtcacagtgtagttatacattctatttttgtccccttttccctttttctcctgtatcttttaaaatttggaaactacttttccagaaggcattatttatgcctccctaataaggtattttacttatgacagatgaaaaggaaccaggatatgtttgaattttttcactttcttagtctgtgacaagaagtagaaatatcactagtgtggtataggaacttacatgttttttatgatgaaaataattctcaatgccacttgaaaggtaattgtgtctgagagctgcaaatttttcaaccacaaaatgtcacttattcctacaggctatacagaggtctttatggtttttttgttttgttttaatggcaacattgtaactgtcaaactaaaagggtattctgtgattatcttttaagcattacagaaattcaagtgaaagttatatgcttatttctattgatgttaaaaatgataatgaaagcaaaattagctgtatctgtaattttctctctagtgccaaatgaatgccttagctactcatagtgcatggtactgtaagtgaagacctgtagctttttttttttctttaatgaaaagcattataatgatgtagcagcatcagatataaacttaaaaaaaaaggtttcaattaacattttatatatggataatgctttgtaaagtgtaagagaaaggttgcagttggatcagtataaaacaatgaccaagccaaaatcagcaccctagggccttaaataaaatagagataccccacaaaatgaaatattttgaaggatgggaggggacagaaggggggactatcccccaagga

SEQ ID NO: 133 - DNA, 37 bp, Homo sapiens
gcagcagcggccgcatgaaggaagactgtctgccgag

SEQ ID NO: 134 - DNA, 1364 bp, Homo sapiens
ggaaaagcgaccttttctgagcgcgtttgcctgttgagtggtagcctttcccctcaaccagcaatggaggagcagccccagatgcaagacgccgacgagcccgcggactccggaggggaaggccgggcaggcgggccaccgcaggtcgccggcgcccaggcggcgtgcagcgaggaccgcatgaccctgctcctcaggctgagagcacagacaaaacaacaactcttagaatataaatcaatggttgatgcaagtgaagaaaaaactccagaacaaattatgcaagaaaagcaaatcgaagctaaaattgaagacctggaaaatgaaattgaagaggtaaaagttgcttttgagataaaaaagcttgcattagacaggatgagactttcaactgcacttaaaaaaaacctggagaaaattagcagacagtctagtgtgctcatggataacatgaaacacctattagagctaaataaattaataatgaaatcacagcaggaatcttgggatttagaggaaaaactgcttgatattagaaagaagagattgcaattaaaacaagcttcagaaagtaagcttttagaaatacagactgaaaagaacaaacagaagattgatttggacagtatggaaaactcagagaggataaagatcatacgacaaaacctacagatggagataaaaattactactgttattcaacatgtgttccagaaccttattttggggagtaaagtcaattgggcagaggatcctgcccttaaggaaattgttctgcagcttgagaagaatgttgacatgatgtaataagaattcatttctgacatattttacatttctggcaatctcaactcttatttggaatacttctgtgcatttgtctgtccaccgtaattttagaaaagcatatccataacgtttacagttgtagtacagttgtggttagttatttgtagtgggattgaaagtaatttttttctttttatatttctatatttagtttgtttttttgttgttgttgttttttgagatggagtctcgctttgttgcccagactggagggcagtggcgcgatctcggctcactgcaacctctgcctcccgggttcaagcagttctgcctcagcctcccaagtagctgtgactaaaggtgcacgccgccatgcccagctaattttttgtattttagtagagacggggtttcaccgtgttgcccaggctgctctcagaactcctgagctcaggcagtccaccgcctcggcctaccgaagtgctaggattacagacgtaagccaccgagcctggtctagtttgcattttttttctatcagttttataagttaagaaataaaaggaattaatgtt

SEQ ID NO: 135 - DNA, 2215 bp, Homo sapiens
cgttattggagccaggcctacaccccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccat

SEQ ID NO: 136 - DNA, 495 bp, Homo sapiens
ccaaggtgctcggtccttccgaggaagctaaggctgcgttggggtgaggccctcacttcatccggcgactagcaccgcgtccggcagcgccagccctacactcgcccgcgccatggcctctgtctccgagctcgcctgcatctactcggccctcattctgcacgacgatgaggtgacagtcacggaggataagatcaatgccctcattaaagcagccggtgtaaatgttgagcctttttggcctggcttgtttgcaaaggccctggccaacgtcaacattgggagcctcatctgcaatgtaggggccggtggacctgctccagcagctggtgctgcaccagcaggaggtcctgccccctccactgctgctgctccagctgaggagaagaaagtggaagcaaagaaagaagaatccgaggagtctgatgatgacatgggctttggtctttttgactaaacctcttttataacatgttcaataaaaagctgaacttt

SEQ ID NO: 137 - DNA, 3393 bp, Homo sapiens
gagatcagcgctgggacggaacccgggttcctctcgaaccgggattgtgacgcttttggcctggctggccgctgttttctgtcccactttttactcgggcctgcgtccgctgccgccgtccctcagtttgcccccggaggaggcagggcggccgtgccttctgccgtgcgcccgcgtggctgccaccgcccctccgaatcctccggggccgcagaggggttcgctacggagggaggtgggggccttcgggaggaggaggcggaggaggcggaggaggagggaaggaagatggcggccgtggaactagagtggatcccagagactctctataacaccgccatctccgctgtcgtggacaactacatccgctcccgccgagacatccgctccttgcccgagaacatccagtttgatgtttactacaagctttaccaacagggacgcttatgtcaactgggcagtgaattttgtgaattggaagtttttgctaaagtactgagagctttggataaaagacatttgcttcatcattgttttcaggctttgatggatcatggtgttaaagttgcttcagtcttggcctactcattcagtaggcggtgctcttatatagcagaatcagatgctgcagtaaaggaaaaagccattcaggttggctttgttttaggtggctttctttcagatgcaggctggtacagtgatgctgagaaagtttttctgtcctgccttcagttgtgtactctacacgatgagatgcttcattggtttcgtgcagtagaatgttgtgtgaggttgcttcatgtgcgaaatggaaactgcaaatatcatttgggtgaagaaacatttaaattagctcagacatatatggataaactatcaaaacatggccagcaagcaaataaagctgcactctatggagaactgtgtgcactcctatttgcaaaaagtcactatgatgaggcatacaaatggtgcatcgaggcaatgaaagaaattacagcaggcttaccagtgaaagttgtggtggatgtcttaagacaagcttctaaggcttgtgtagtaaaacgtgaatttaagaaggcagaacagttaattaaacatgcagtgtatttggcacgggatcattttggatccaaacacccaaaatattctgatacactgctagattatgggttctacttactcaatgtagataatatctgtcagtctgttgcaatttatcaggcagcccttgacattagacagtcagtgtttggtggcaaaaatatccacgtagcaacagctcatgaagatttggcctactcttcttatgtccaccagtatagctctgggaaatttgacaatgcactatttcatgcagaaagagctattggtatcattacccacatcctacctgaagatcatcttcttttggcttcttcaaagagggtgaaagcacttattttagaggagattgcaattgattgtcataataaggaaactgaacagaggctgcttcaagaagctcatgatttgcacctgtcttcactccaactagctaaaaaagcttttggggaatttaatgtacagactgcaaaacactatggaaaccttggaagactttatcagtcaatgagaaaatttaaggaagctgaagaaatgcacatcaaagcaattcagattaaagaacaacttcttggtcaagaagattatgaagtagccctttcagtgggacatctggcttctttatataattatgacatgaatcagtatgaaaatgctgagaaactttatttgcgatctatagcaattgggaagaaactttttggtgagggctacagtggactagaatatgattatcgaggtctcattaaactttacaactccattggaaattacgagaaagtgtttgaatatcacaatgttctgtctaactggaaccggttgcgagatcggcaatattcagtgacagatgctcttgaagatgtcagcaccagcccccagtccactgaagaagtggtgcagtccttcctgatttctcagaatgtcgagggaccgagctgctgagggaggacctcagttaaccaattaccttttcccggattccagggaattcatactgtgaaatcaaaaccatgttgttttggggggctggaatttgcattgaaacactggtccagtccattgaagaccctattttgggtgatccctatcttgcagaatgtctgtaggaataagcatatattcagttatattcagcatgtaccgcatgtgtaagtagtctggcccacattttcaacctagtagaacaaacaacaggaaatcttttttttgttgtttttaaaaaattcattttgcagaaagcctgaaagaaaaaaaatacccctaaataaaactatttaagagtttaaaagagttgcattcttattatgtaaggatgattttaacaactttttaatatgtaattcttccatgtggaggtattcaatactgtagtgtaaagaaattttatgcggaaaatctttatatgcagtatagaaaagttaacacaagtactaataaaagagggacatcccgacttacgtttttctaccttgcccagataagtggatacaaccactctatattacaaggaaaggactgtcagattcatctgaactggaccagtgttgatctgtaatgtaatagaaaatctgatagaccagcacttctgacttttttttttttggtacaacaatgcaagatgctctgatagcatttgctaacaggaccaggaggatctaaaaaggaccagcctaatgtagaaggtggttacttggaccagaggctttagattattattttagatcctacatatacttttatcagtagaatgatttcatttagatgtataatgaaaaaggataatgcaaaaattatgtaatagataccaaattagggaagtttggcaatttcaatggcatatttttagtcaaggtacacagatggcagtgccataagcaagtctataaatatcggctgcagccatccccctcattttaaatgttgccctaataatcaatgcagttaacaagtatattggctgtgtgtcatgaaatagttcatgttcagatggaaatgttaggttactgtatggtttatggagattaatgaaaatgaatgcccaaaaataagtcttagaaaatcctccatttttatggtaaatagtaatacaactaggtcatttcatttgaaatctaggagtcaaatggaaagatcccctaataatacacctatttcactaacttgtctttctgtttattgggttttgatttgattttttgtaagccagtcaggttatttaatgatgaggtaataatcaaatttaagaatttgtgacatgtagcaattcaagaaacaaaaaggtattttgct

SEQ ID NO: 138 - DNA, 2618 bp, Homo sapiens
atggcggcgggtgggagcggcgggcgtgcgtcgtgcccgccgggggtcggggtcggcccgggcacggggggcagtcccgggcccagcgccaacgccgccgccaccccggcccccggcaacgcggccgccgccgccgccgccgccgccgccgccgccgccgcccctgggccgacgccgcccgccccgccgggccccgggacagacgcgcaggccgcgggcgcggagcgggcggaggaggcggcgggcccgggggcggcggcgctgcagcgcgaggccgcgtacaactggcaggccagcaagcccaccgtgcaggagcgcttcgccttcctcttcaacaacgaggtgctgtgcgacgtgcacttcctggtgggcaaggggctcagctcgcagcgcatccccgcgcacaggttcgtgctggccgtgggcagcgccgtctttgatgccatgttcaacgggggaatggccacaacatccacggagattgagctgcccgacgtggaacccgctgccttcctcgcactgctcaagtttctctactcggacgaggtgcagattggcccggagacggtgatgaccacgctatacaccgccaagaagtacgcggtgccagcgctcgaggcccattgcgtggagttcctgaagaagaacctgcgagccgacaacgccttcatgctgctcacgcaggcgcgactcttcgatgaaccgcagctggccagcctgtgcctggagaacatcgacaaaaacactgcagacgccatcaccgcggagggcttcaccgacattgacctggacacgctggtggctgtcctggagcgcgacacactgggcatccgtgaggtgcggctgttcaatgccgttgtccgctggtccgaggccgagtgtcagcggcagcagctgcaggtgacgccagagaacaggcggaaggttctgggcaaggccctgggcctcattcgcttcccgctcatgaccatcgaggagttcgctgcaggtcccgcacagtcgggcatcctggtggaccgcgaggtggtcagcctcttcctgcacttcaccgtcaaccccaagccacgagtggagttcattgaccggccccgctgctgcctgcgtgggaaggagtgcagcatcaaccgcttccagcaggtggagagtcgctggggctacagcgggaccagtgaccgcatcaggttctcagtcaacaagcgcatcttcgtggtgggatttgggctgtatggatccatccacgggcccaccgactaccaagtgaacatccagattattcacaccgatagcaacaccgtcttgggccagaacgacacgggcttcagctgcgacggctcagccagcaccttccgcgtcatgttcaaggagccggtggaggtgctgcccaacgtcaactacacggcctgtgccacgctcaagggcccagactcccactacggcaccaaaggcctgcgcaaggtgacacacgagtcgcccaccacgggcgccaagacctgcttcaccttttgctacgcggccgggaacaacaatggcacatccgtggaggacggccagatccccgaggtcatcttctacacctaggctgcccgacaccgacaccgccctccctccgtggggatagccgcagccccaggccatcatctgctgctggggcccccccaccacgcggtgccaggcccagtgtcccccaggccgtctgtccactccatgccacctttctcagcatcaggacggggttgccctgtgttcaccacgagtgtggctgctggatcagggcagccggggaggtggccaggccagtggccaggccctgtggagacaatccctcaggactagggacagggctgtgccggcctgggccagggcccacggacccgcagctcagggcgcctgcccacgtcgtctgccggcggtgcgccgcgggcgtccctcgcgtctcttcactgcacattgcaatgcatttgcgattcccatttctctgctaggagccagcctgggtggcgctgctcccagagccgtgggtcccagaccttgcgttccttttgttcctgtccgtttatcaggacacgggccccacctgtcacgtgcccgaggccacccaagcccagcctgcggggcgttcccactgcctggatgccggcttgagttctgcgcacgcaggattcagtgtggggacggcccctgccggataggcctagccctggcccaggtggtgagcggtttgcagtgtccgttctcatccacctgatgggcccagataaaggcccccgctgtccagcctccctggacggccctcgcggtccctgcagcccaagatgggactcagaccctgtgccccagagctcccctgccgcagaatggggccccagccggccccgaccgggtccaggagcactgctcgcctgtacatactgttgccctagcccacctggtgccgtgggagccacccccaggtgctgggggcacagcccctccccactccggccacgcccccacccaccccgcgtgtttctgccctgtgactcctggaacctgcgtcctccccaaagccatgggaggggtgtcctcctcagaccatgcccccagatgatttttttaaataaagaaacaaatgcacctgc

SEQ ID NO: 139 - DNA, 1378 bp, Homo sapiens
ggaaactgctccgcgcgcgccgcgggaggaggaaccgcccggtcctttagggtccgggcccggccgggccatggattcaatgcctgagcccgcgtcccgctgtcttctgcttcttcccttgctgctgctgctgctgctgctgctgccggccccggagctgggcccgagccaggccggagctgaggagaacgactgggttcgcctgcccagcaaatgcgaagtgtgtaaatatgttgctgtggagctgaagtcagcctttgaggaaaccggcaagaccaaggaggtgattggcacgggctatggcatcctggaccagaaggcctctggagtcaaatacaccaagtcggacttgcggttaatcgaagtcactgagaccatttgcaagaggctcctggattatagcctgcacaaggagaggaccggcagcaatcgatttgccaagggcatgtcagagacctttgagacattacacaacctggtacacaaaggggtcaaggtggtgatggacatcccctatgagctgtggaacgagacttctgcagaggtggctgacctcaagaagcagtgtgatgtgctggtggaagagtttgaggaggtgatcgaggactggtacaggaaccaccaggaggaagacctgactgaattcctctgcgccaaccacgtgctgaagggaaaagacaccagttgcctggcagagcagtggtccggcaagaagggagacacagctgccctgggagggaagaagtccaagaagaagagcagcagggccaaggcagcaggcggcaggagtagcagcagcaaacaaaggaaggagctgggtggccttgagggagaccccagccccgaggaggatgagggcatccagaaggcatcccctctcacacacagcccccctgatgagctctgagcccacccagcatcctctgtcctgagacccctgattttgaagctgaggagtcaggggcatggctctggcaggccgggatggccccgcagccttcagcccctccttgccttggctgtgccctcttctgccaaggaaagacacaagccccaggaagaactcagagccgtcatgggtagcccacgccgtcctttcccctccccaagtgtttctctcctgacccagggttcaggcaggccttgtggtttcaggactgcaaggactccagtgtgaactcaggaggggcaggtgtcagaactgggcaccaggactggagccccctccggagaccaaactcaccatccctcagtcctccccaacagggtactaggactgcagccccctgtagctcctctctgcttacccctcctgtggacaccttgcactctgcctggcccttcccagagcccaaagagtaaaaatgttctggttctg

SEQ ID NO: 140 - DNA, 2797 bp, Homo sapiens
aagatggcggaccttgattcgcctccgaagctgtcaggggtgcagcagccgtctgagggggtgggaggtggccgctgctccgaaatctccgctgagctcattcgctccctgacagagctgcaggagctggaggctgtatacgaacggctctgcggcgaggagaaagtggtggagagagagctggatgctcttttggaacagcaaaacaccattgaaagtaagatggtcactctccaccgaatgggtcctaatctgcagctgattgagggagatgcaaagcagctggctggaatgatcacctttacctgcaacctggctgagaatgtgtccagcaaagttcgtcagcttgacctggccaagaaccgcctctatcaggccattcagagagctgatgacatcttggacctgaagttctgcatggatggagttcagactgctttgaggagtgaagattatgagcaggctgcagcacatattcatcgctacttgtgcctggacaagtcggtcattgagctcagccgacagggcaaaggggggagcatgattgatgccaacctgaaattgctgcaggaagctgagcaacgtctcaaagccattgtggcagagaagtttgccattgccaccaaggaaggtgatttgccccaggtggagcgcttcttcaagatcttcccactgctgggtttgcatgaggagggattaagaaggttctcggagtacctttgcaagcaggtggccagtaaagctgaggagaatctgctcatggtgctggggacagacatgagtgatcggagagctgcagtcatctttgcagatacacttactcttctgtttgaagggattgcccgcattgtggaggcccaccagccaatagtggagacctattatgggccagggagactctataccctgatcaaatatctgcaggtggaatgtgacagacaggtggagaaggtggtagacaagttcatcaagcaaagggactaccaccagcagttccggcatgttcagaacaacctgatgagaaattctacaacagaaaaaatcgaaccaagagaactggaccccatcctgactgaggtcaccctgatgaacgcccgcagtgagctatacttacgcttcctcaagaagaggattagctctgattttgaggtgggagactccatggcctcagaggaagtaaagcaagagcaccagaagtgtctggacaaactcctcaataactgccttttgagctgtaccatgcaggagctaattggcttatatgttaccatggaggagtacttcatgagggagactgtcaataaggctgtggctctggacacctatgagaagggccagctgacatccagcatggtggatgatgtcttctacattgttaagaagtgcattgggcgggctctgtccagctccagcattgactgtctctgtgccatgatcaacctcgccaccacagagctggagtctgacttcagggatgttctgtgtaataagctgcggatgggctttcctgccaccaccttccaggacatccagcgcggggtgacaagtgccgtgaacatcatgcacagcagcctccagcaaggcaaatttgacacaaaaggcatcgagagtactgacgaggcgaagatgtccttcctggtgactctgaacaacgtggaagtctgcagtgaaaacatctccactctgaagaagacactggagagtgactgcaccaagctcttcagccagggcattggaggggagcaggcccaggccaagtttgacggctgcctttctgacttggccgccgtgtccaacaaattccgagacctcttgcaggaagggctgacggagctcaacagcacagccatcaagccacaggtgcagccttggatcaacagctttttctccgtctcccacaacatcgaggaggaagaattcaatgactatgaggccaacgacccttgggtacaacagttcatccttaacctggagcagcaaatggcagagttcaaggccagcctgtccccggtcatctacgacagcctaaccggcctcatgactagccttgttgccgtcgagttggagaaagtggtgctgaaatccacctttaaccggctgggtggtctgcagtttgacaaggagctgaggtcgctcattgcctaccttaccacggtgaccacctggaccatccgagacaagtttgcccggctctcccagatggccaccatcctcaatctggagcgggtgaccgagatcctcgattactggggacccaattccggcccattgacgtggcgcctcacccctgctgaagtgcgccaggtgctggccctgcggatagacttccgcagtgaagatatcaagaggctgcgcctgtagctgcctggatgagcacacctggctcatcacacttgcaggcctgttccctaaggggccccagccaaggagctgagcgaggctgtctggcttgggggagatctgacagcccagacctttctacggctggcagcagagaaacaaagtctggacccactccatgctctgccctcagacctggccaggtgatgctctgggggcagcatctccccaccgagagaagcgggctcctaatgaggtgggaaagccacggcaggcagcgagcagcccaggccagctttctgcatggatggtcagtctcttgccctcaaacactacagcaaacaagctacccctgccagtcctagacaacttgggtacatctggggacctagcagttaggcttgactttgaggagaggctgtgatgtttatgatccctgaataaagctactccttgg

SEQ ID NO: 141 - protein, 91 aa, Homo sapiens
HFYNHKTSVFTPAYGSVTNVRVNSTMTTLQVLTLLLNKFRVEDGPSEFALYIVHESGERTKLKDCEYPLISRILHGPCEKIARIFLMEADL

SEQ ID NO: 142 - protein, 145 aa, Homo sapiens
TIDHSLIPEERLSPTPSMQDGLDLPSETDLRILGCELIQAAGILLRLPQVAMATGQVLFHRFFYSKSFVKHSFEIVAMACINLASKIEEAPRRIRDVINVFHHLRQLRGKRTPSPLILDQNYINTKNQVIKAERRVLKELGFCVH

SEQ ID NO: 143 - protein, 19 aa, Homo sapiens
PETIACACIYLAARALQIP

SEQ ID NO: 144 - protein, 627 aa, Homo sapiens
MEGLAGYVYKAASEGKVLTLAALLLNRSESDIRYLLGYVSQQGGQRSTPLIIAARNGHAKVVRLLLEHYRVQTQQTGTVRFDGYVIDGATALWCAAGAGHFEVVKLLVSHGANVNHTTVTNSTPLRAACFDGRLDIVKYLVENNANISIANKYDNTCLMIAAYKGHTDVVRYLLEQRADPNAKAHCGATALHFAAEAGHIDIVKELIKWRAAIVVNGHGMTPLKVAAESCKADVVELLLSHADCDRRSRIEALELLGASFANDRENYDIIKTYHYLYLAMLERFQDGDNILEKEVLPPIHAYGNRTECRNPQELESIRQDRDALHMEGLIVRERILGADNIDVSHPIIYRGAVYADNMEFEQCIKLWLHALHLRQKGNRNTHKDLLRFAQVFSQMIHLNETVKAPDIECVLRCSVLEIEQSMNRVKNISDADVHNAMDNYECNLYTFLYLVCISTKTQCSEEDQCKINKQIYNLIHLDPRTREGFTLLHLAVNSNTPVDDFHTNDVCSFPNALVTKLLLDCGAEVNAVDNEGNSALHIIVQYNRPISDFLTLHSIIISLVEAGAHTDMTNKQNKTPLDKSTTGVSEILLKTQMKMSLKCLAARAVRANDINYQDQIPRTLEEFVGFH

SEQ ID NO: 145 - DNA, 37 bp, Homo sapiens
gcagcagtcgacttttaatttttcaacaaaactgtcc

SEQ ID NO: 146 - protein, 247 aa, Homo sapiens
MEEQPQMQDADEPADSGGEGRAGGPPQVAGAQAACSEDRMTLLLRLRAQTKQQLLEYKSMVDASEEKTPEQIMQEKQIEAKIEDLENEIEEVKVAFEIKKLALDRMRLSTALKKNLEKISRQSSVLMDNMKHLLELNKLIMKSQQESWDLEEKLLDIRKKRLQLKQASESKLLEIQTEKNKQKIDLDSMENSERIKIIRQNLQMEIKITTVIQHVFQNLILGSKVNWAEDPALKEIVLQLEKNVDMM

SEQ ID NO: 147 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 148 - protein, 113 aa, Homo sapiens
MASVSELACIYSALILHDDEVTVTEDKINALIKAAGVNVEPFWPGLFAKALANVNIGSLICNVGAGGPAPAAGAAPAGGPAPSTAAAPAEEKKVEAKKEESEESDDDMGFGLF

SEQ ID NO: 149 - protein, 681 aa, Homo sapiens
EISAGTEPGFLSNRDCDAFGLAGRCFLSHFLLGPASAAAVPQFAPGGGRAAVPSAVRPRGCHRPSESSGAAEGFATEGGGGLREEEAEEAEEEGRKMAAVELEWIPETLYNTAISAVVDNYIRSRRDIRSLPENIQFDVYYKLYQQGRLCQLGSEFCELEVFAKVLRALDKRHLLHHCFQALMDHGVKVASVLAYSFSRRCSYIAESDAAVKEKAIQVGFVLGGFLSDAGWYSDAEKVFLSCLQLCTLHDEMLHWFRAVECCVRLLHVRNGNCKYHLGEETFKLAQTYMDKLSKHGQQANKAALYGELCALLFAKSHYDEAYKWCIEAMKEITAGLPVKVVVDVLRQASKACVVKREFKKAEQLIKHAVYLARDHFGSKHPKYSDTLLDYGFYLLNVDNICQSVAIYQAALDIRQSVFGGKNIHVATAHEDLAYSSYVHQYSSGKFDNALFHAERAIGIITHILPEDHLLLASSKRVKALILEEIAIDCHNKETEQRLLQEAHDLHLSSLQLAKKAFGEFNVQTAKHYGNLGRLYQSMRKFKEAEEMHIKAIQIKEQLLGQEDYEVALSVGHLASLYNYDMNQYENAEKLYLRSIAIGKKLFGEGYSGLEYDYRGLIKLYNSIGNYEKVFEYHNVLSNWNRLRDRQYSVTDALEDVSTSPQSTEEVVQSFLISQNVEGPSC

SEQ ID NO: 150 - protein, 525 aa, Homo sapiens
MAAGGSGGRASCPPGVGVGPGTGGSPGPSANAAATPAPGNAAAAAAAAAAAAAAPGPTPPAPPGPGTDAQAAGAERAEEAAGPGAAALQREAAYNWQASKPTVQERFAFLFNNEVLCDVHFLVGKGLSSQRIPAHRFVLAVGSAVFDAMFNGGMATTSTEIELPDVEPAAFLALLKFLYSDEVQIGPETVMTTLYTAKKYAVPALEAHCVEFLKKNLRADNAFMLLTQARLFDEPQLASLCLENIDKNTADAITAEGFTDIDLDTLVAVLERDTLGIREVRLFNAVVRWSEAECQRQQLQVTPENRRKVLGKALGLIRFPLMTIEEFAAGPAQSGILVDREVVSLFLHFTVNPKPRVEFIDRPRCCLRGKECSINRFQQVESRWGYSGTSDRIRFSVNKRIFVVGFGLYGSIHGPTDYQVNIQIIHTDSNTVLGQNDTGFSCDGSASTFRVMFKEPVEVLPNVNYTACATLKGPDSHYGTKGLRKVTHESPTTGAKTCFTFCYAAGNNNGTSVEDGQIPEVIFYT

SEQ ID NO: 151 - protein, 278 aa, Homo sapiens [sequence omitted]

SEQ ID NO: 152 - protein, 785 aa, Homo sapiens
MADLDSPPKLSGVQQPSEGVGGGRCSEISAELIRSLTELQELEAVYERLCGEEKVVERELDALLEQQNTIESKMVTLHRMGPNLQLIEGDAKQLAGMITFTCNLAENVSSKVRQLDLAKNRLYQAIQRADDILDLKFCMDGVQTALRSEDYEQAAAHIHRYLCLDKSVIELSRQGKGGSMIDANLKLLQEAEQRLKAIVAEKFAIATKEGDLPQVERFFKIFPLLGLHEEGLRRFSEYLCKQVASKAEENLLMVLGTDMSDRRAAVIFADTLTLLFEGIARIVEAHQPIVETYYGPGRLYTLIKYLQVECDRQVEKVVDKFIKQRDYHQQFRHVQNNLMRNSTTEKIEPRELDPILTEVTLMNARSELYLRFLKKRISSDFEVGDSMASEEVKQEHQKCLDKLLNNCLLSCTMQELIGLYVTMEEYFMRETVNKAVALDTYEKGQLTSSMVDDVFYIVKKCIGRALSSSSIDCLCAMINLATTELESDFRDVLCNKLRMGFPATTFQDIQRGVTSAVNIMHSSLQQGKFDTKGIESTDEAKMSFLVTLNNVEVCSENISTLKKTLESDCTKLFSQGIGGEQAQAKFDGCLSDLAAVSNKFRDLLQEGLTELNSTAIKPQVQPWINSFFSVSHNIEEEEFNDYEANDPWVQQFILNLEQQMAEFKASLSPVIYDSLTGLMTSLVAVELEKVVLKSTFNRLGGLQFDKELRSLIAYLTTVTTWTIRDKFARLSQMATILNLERVTEILDYWGPNSGPLTWRLTPAEVRQVLALRIDFRSEDIKRLRL

SEQ ID NO: 153 - protein, 527 aa, Rattus norvegicus
MASGPHPTSTAAAASASSAAPSAGGSSSGTTTTTTTTTGGILIGDRLYSEVSLTIDHSVIPEERLSPTPSMQDGLDLPSETDLRILGCELIQAAGILLRLPQVAMATGQVLFHRFFYSKSFVKHSFEIVAMACINLASKIEEAPRRIRDVINVFHHLRQLRGKRTPSPLILDQNYINTKNQVIKAERRVLKELGFCVHVKHPHKIIVMYLQVLECERNQTLVQTAWNYMNDSLRTNVFVRFQPETIACACIYLAARALQIPLPTRPHWFLLFGTTEEEIQEICIETLRLYTRKKPNYELLEKEVEKRKVALQEAKLKAKGLNLDGTPALSTLGGFSPASKPSSPREVKAEEKSPVSINVKTVKKEPEDRQQASKSPYNGVRKDSKRSRNSRSASRSRSRTRSRSRSHTPRRHYNNRRSRSGTYSSRSRSRSRSHSESPRRHHNHGSPHLKAKHTREDLKSSNRHGHKRKKSRSRSQSKTRDHSDVTKKHRHERGHHRDRRERSRSFERSHKGKHHGGSRSGHGRHRR

SEQ ID NO: 154 - protein, 531 aa, Mus musculus
MASGPHPTSTAAAAAAAAASASSAAPSAALPAPARPPRRRPRPEDPDRRPLYSEVSLTIDHSLIPEERLSPTPSMQDGLDLPSETDLRILGCELIQAAGILLRLPQVAMATGQVLFHRFFYSKSFVKHSFEIVAMACINLASKIEEAPRRIRDVINVFHHLRQLRGKRTPSPLILDQNYINTKNQVIKAERRVLKELGFCVHVKHPHKIIVMYLQVLECERNQTLVQTAWNYMNDSLRTNVFVRFQPETIACACIYLAARALQIPLPTRPHWFLLFGTTEEEIQEICIETLRLYTRKKPNYELLEKEVEKRKVALQEAKLKAKGLNLDGTPALSTLGGFSPASKPSSPREVKAEEKSPVSINVKTVKKEPEDRQQASKSPYNGVRKDSKRSRTSRSASRSRSRTRSRSRSHSPRRHYNNRRSRSGTYSSRSRSRSRSHSESPRRHHNHGSPHLKAKHTREDLKSSNRHGHKRKKSRSRSQSKTRDHSDVTKKHRHERGHHRDRRERSRSFERSHKGKHHGGSRSGHGRHRR

SEQ ID NO: 155 - protein, 520 aa, Homo sapiens
MAAAAAAAGAAGSAAPAAAAGAPGSGGAPSGSQGVLIGDRLYSGVLITLENCLLPDDKLRFTPSMSSGLDTDTETDLRVVGCELIQAAGILLRLPQVAMATGQVLFQRFFYTKSFVKHSMEHVSMACVHLASKIEEAPRRIRDVINVFHRLRQLRDKKKPVPLLLDQDYVNLKNQIIKAERRVLKELGFCVHVKHPHKIIVMYLQVLECERNQHLVQTSWNYMNDSLRTDVFVRFQPESIACACIYLAARTLEIPLPNRPHWFLLFGATEEEIQEICLKILQLYARKKVDLTHLEGEVEKRKHAIEEAKAQARGLLPGGTQVLDGTSGFSPAPKLVESPKEGKGSKPSPLSVKNTKRRLEGAKKAKADSPVNGLPKGRESRSRSRSREQSYSRSPSRSASPKRRKSDSGSTSGGSKSQSRSRSRSDSPPRQAPRSAPYKGSEIRGSRKSKDCKYPQKPHKSRSRSSSRSRSRSRERADNPGKYKKKSHYYRDQRRERSRSYERTGRRYERDHPGHSRHRR

SEQ ID NO: 156 - protein, 560 aa, Drosophila melanogaster
MATRGAGSTVVHTTVTALTVETITNVLTTVTSFHSNSVNISNNNSSSGAAPGADAAGGDAGGVAAAQADANKPIYPRLFNRIVLTLENSLIPEGKIDVTPSSQDGLDHETEKDLRILGCELIQTAGILLRLPQVAMATGQVLFQRFFYSKSFVRHNMETVAMSCVCLASKIEEAPRRIRDVINVFHHIKQVRAQKEISPMVLDPYYTNLKMQVIKAERRVLKELGFCVHVKHPHKLIVMYLQVLQYEKHEKLMQLSWNFMNDSLRTDVFMRYTPEAIACACIYLSARKLNIPLPNSPPWFGIFRVPMADITDICYRVMELYMRSKPVVEKLEAAVDELKKRYIDARNKTKEANTPPAVITVDRNNGSHNAWGGFIQRAIPLPLPSEKSPQKDSRSRSRSRTRTHSRTPRSRSPRSRSPSRERTKKTHRSRSSRSRSRSPPKHKKKSRHYSRSPTRSNSPHSKHRKSKSSRERSEYYSKKDRSGNPGSSNNLGDGDKYRNSVSNSGKHSRYSSSSSRRNSGGGGDGRSGGGGGGGGGGNGNHGSRGGHKHRDGDRSRDRKR

SEQ ID NO: 157 - protein, 469 aa, Homo sapiens
MATRGAGSTVVHTTVTALTVETITNVLTTVTSFHSNSVNISNNNSSSGAAPGADAAGGDAGGVAAAQADANKPIYPRLFNRIVLTLENSLIPEGKIDVTPSSQDGLDHETEKDLRILGCELIQTAGILLRLPQVAMATGQVLFQRFFYSKSFVRHNMETVAMSCVCLASKIEEAPRRIRDVINVFHHIKQVRAQKEISPMVLDPYYTNLKMQVIKAERRVLKELGFCVHVKHPHKLIVMYLQVLQYEKHEKLMQLSWNFMNDSLRTDVFMRYTPEAIACACIYLSARKLNIPLPNSPPWFGIFRVPMADITDICYRVMELYMRSKPVVEKLEAAVDELKKRYIDARNKTKEANTPPAVITVDRNNGSHNAWGGFIQRAIPLPLPSEKSPQKDSRSRSRSRTRTHSRTPRSRSPRSRSPSRERTKKTHRSRSSRSRSRSPPKHKKKSRHYSRSPTRSNSPHSKHRKSYVL

SEQ ID NO: 158 - DNA, 1171 bp, Homo sapiens
gcggccgcggcgtctcctccgggacgctgaggggcccgaggagaccgtgaggctctggcctgcagctcgcgccgccatggacgctgccgaggtcgaattcctcgccgagaaggagctggttaccattatccccaacttcagtctggacaagatctacctcatcgggggggacctggggccttttaaccctggtttacccgtggaagtgcccctgtggctggcgattaacctgaaacaaagacagaaatgtcgcctgctccctccagagtggatggatgtagaaaagttggagaagatgagggatcatgaacgaaaggaagaaacttttaccccaatgcccagcccttactacatggaacttacgaagctcctgttaaatcatgcttcagacaacatcccgaaggcagacgaaatccggaccctggtcaaggatatgtgggacactcgtatagccaaactccgagtgtctgctgacagctttgtgagacagcaggaggcacatgccaagctggataacttgaccttgatggagatcaacaccagcgggactttcctcacacaagcgctcaaccacatgtacaaactccgcacgaacctccagcctctggagagtactcagtctcaggacttctagagaaaggcctggtgcaggcggcttgctgggggatgtgagcgctcaggacgtgatgaggtactcgtggttctggagctctagaaacacttctgatgcatgaaaaatgtgtgatggtgcaaggaatggattcaggatgttgttggagaaacaagtttgtgattagtccttaaaacttagctccctgggacattcttcaattccacatctgtttctagaaaccagccctttttccccccacttttgagaaataaaaaagccttaggtaaataagtcattctccctagcagagccacttgggtctcctgcatggaagccgtcacacttgggcaggtgttcagtgactggtaggtgtagatacagcaggagtggccatgtggtccacggctttttaccccttcttgatcctgatttcttgggctgaatttagactctctcacagaggtggctcacagagaaggatggcagatggtgcagccaacaatgctgaccggtgcttatcctctaagccctgatccacaataaaaatggacccaactc

SEQ ID NO: 159 - DNA, 1523 bp, Homo sapiens
gtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaagg

SEQ ID NO: 160 - protein, 185 aa, Homo sapiens
MDAAEVEFLAEKELVTIIPNFSLDKIYLIGGDLGPFNPGLPVEVPLWLAINLKQRQKCRLLPPEWMDVEKLEKMRDHERKEETFTPMPSPYYMELTKLLLNHASDNIPKADEIRTLVKDMWDTRIAKLRVSADSFVRQQEAHAKLDNLTLMEINTSGTFLTQALNHMYKLRTNLQPLESTQSQDF

SEQ ID NO: 161 - protein, 249 aa, Homo sapiens [sequence omitted]

SEQ ID NO: 162 - DNA, 24 bp, Homo sapiens
ccattcagaagtcggagctcttag

SEQ ID NO: 163 - DNA, 21 bp, Homo sapiens
gaagctcttgccctcatggta

SEQ ID NO: 164 - DNA, 22 bp, Homo sapiens
gcttgcatctaccttgcagcta

SEQ ID NO: 165 - DNA, 20 bp, Homo sapiens
acgagttggcaacggaatct

SEQ ID NO: 166 - DNA, 17 bp, Homo sapiens
agccgagccacatcgct

SEQ ID NO: 167 - DNA, 19 bp, Homo sapiens
gtgaccaggcgcccaatac

SEQ ID NO: 168 - DNA, 39 bp, Homo sapiens
gcagcagcggccgccccatcagtgacagcaagtccattc

SEQ ID NO: 169 - DNA, 37 bp, Homo sapiens
gcagcagtcgaccttggcctccaccagctgctccagg

SEQ ID NO: 170 - DNA, 23 bp, Homo sapiens
caggtgcagctggtgcagtctgg

SEQ ID NO: 171 - DNA, 23 bp, Homo sapiens
caggtcaacttaagggagtctgg

SEQ ID NO: 172 - DNA, 23 bp, Homo sapiens
gaggtgcagctggtggagtctgg

SEQ ID NO: 173 - DNA, 23 bp, Homo sapiens
caggtgcagctgcaggagtcggg

SEQ ID NO: 174 - DNA, 23 bp, Homo sapiens
gaggtgcagctgttgcagtctgc

SEQ ID NO: 175 - DNA, 23 bp, Homo sapiens
caggtacagctgcagcagtcagg

SEQ ID NO: 176 - DNA, 24 bp, Homo sapiens
tgaggagacggtgaccagggtgcc

SEQ ID NO: 177 - DNA, 24 bp, Homo sapiens
tgaagagacggtgaccattgtccc

SEQ ID NO: 178 - DNA, 24 bp, Homo sapiens
tgaggagacggtgaccagggttcc

SEQ ID NO: 179 - DNA, 24 bp, Homo sapiens
tgaggagacggtgaccgtggtccc

SEQ ID NO: 180 - DNA, 23 bp, Homo sapiens
gacatccagatgacccagtctcc

SEQ ID NO: 181 - DNA, 23 bp, Homo sapiens
gatgttgtgatgactcagtctcc

SEQ ID NO: 182 - DNA, 23 bp, Homo sapiens
gatattgtgatgactcagtctcc

SEQ ID NO: 183 - DNA, 23 bp, Homo sapiens
gaaattgtgttgacgcagtctcc

SEQ ID NO: 184 - DNA, 23 bp, Homo sapiens
gacatcgtgatgacccagtctcc

SEQ ID NO: 185 - DNA, 23 bp, Homo sapiens
gaaacgacactcacgcagtctcc

SEQ ID NO: 186 - DNA, 23 bp, Homo sapiens
gaaattgtgctgactcagtctcc

SEQ ID NO: 187 - DNA, 23 bp, Homo sapiens
cagtctgtgttgacgcagccgcc

SEQ ID NO: 188 - DNA, 23 bp, Homo sapiens
cagtctgccctgactcagcctgc

SEQ ID NO: 189 - DNA, 23 bp, Homo sapiens
tcctatgtgctgactcagccacc

SEQ ID NO: 190 - DNA, 23 bp, Homo sapiens
tcttctgagctgactcaggaccc

SEQ ID NO: 191 - DNA, 23 bp, Homo sapiens
cacgttatactgactcaaccgcc

SEQ ID NO: 192 - DNA, 23 bp, Homo sapiens
caggctgtgctcactcagccgtc

SEQ ID NO: 193 - DNA, 23 bp, Homo sapiens
aattttatgctgactcagcccca

SEQ ID NO: 194 - DNA, 24 bp, Homo sapiens
acgtttgatttccaccttggtccc

SEQ ID NO: 195 - DNA, 24 bp, Homo sapiens
acgtttgatctccagcttggtccc

SEQ ID NO: 196 - DNA, 24 bp, Homo sapiens
acgtttgatatccactttggtccc

SEQ ID NO: 197 - DNA, 24 bp, Homo sapiens
acgtttgatctccaccttggtccc

SEQ ID NO: 198 - DNA, 24 bp, Homo sapiens
acgtttaatctccagtcgtgtccc

SEQ ID NO: 199 - DNA, 23 bp, Homo sapiens
cagtctgtgttgacgcagccgcc

SEQ ID NO: 200 - DNA, 23 bp, Homo sapiens
cagtctgccctgactcagcctgc

SEQ ID NO: 201 - DNA, 23 bp, Homo sapiens
tcctatgtgctgactcagccacc

SEQ ID NO: 202 - DNA, 23 bp, Homo sapiens
tcttctgagctgactcaggaccc

SEQ ID NO: 203 - DNA, 23 bp, Homo sapiens
cacgttatactgactcaaccgcc

SEQ ID NO: 204 - DNA, 23 bp, Homo sapiens
caggctgtgctcactcagccgtc

SEQ ID NO: 205 - DNA, 23 bp, Homo sapiens
aattttatgctgactcagcccca

SEQ ID NO: 206 - DNA, 24 bp, Homo sapiens
ggtctttcctccagtgtcacaata

SEQ ID NO: 207 - DNA, 24 bp, Homo sapiens
gaacctcctacctttcaggcacta

SEQ ID NO: 208 - DNA, 23 bp, Homo sapiens
cacatgcaacatttggattcagt

SEQ ID NO: 209 - DNA, 26 bp, Homo sapiens
acggttactttcttgtgagtctttga

SEQ ID NO: 210 - DNA, 22 bp, Homo sapiens
gagacaatgcgaatgcaaagag

SEQ ID NO: 211 - DNA, 24 bp, Homo sapiens
ccaccatatctgacccaagagagt

SEQ ID NO: 212 - DNA, 22 bp, Homo sapiens
ggaaggatgaagcggagaaagt

SEQ ID NO: 213 - DNA, 25 bp, Homo sapiens
gactgagtccagagaaatgtgtgaa

SEQ ID NO: 214 - DNA, 19 bp, Homo sapiens
ggctggcaattcgaaagga

SEQ ID NO: 215 - DNA, 24 bp, Homo sapiens
ggaatcaccatcagcttgtttagc

SEQ ID NO: 216 - DNA, 27 bp, Homo sapiens
ggtccttgatgtcgatattcttaacac

SEQ ID NO: 217 - DNA, 26 bp, Homo sapiens
ccatgctttagttgccatttacttct

SEQ ID NO: 218 - DNA, 22 bp, Homo sapiens
ttgcaagtcttggatgtggttt

SEQ ID NO: 219 - DNA, 22 bp, Homo sapiens
ctggcacgtaatggtcactgtt

SEQ ID NO: 220 - DNA, 21 bp, Homo sapiens
gctgatggaagggagtcaaca

SEQ ID NO: 221 - DNA, 24 bp, Homo sapiens
ctccataagggagctcacctactt

SEQ ID NO: 222 - DNA, 24 bp, Homo sapiens
gtggtacagtgcaatgtcttccat

SEQ ID NO: 223 - DNA, 23 bp, Homo sapiens
catgacctttgcaagacctccta

SEQ ID NO: 224 - DNA, 21 bp, Homo sapiens
ccacagtagccatgggtcaat

SEQ ID NO: 225 - DNA, 20 bp, Homo sapiens
ctatggcagggcttggacaa

SEQ ID NO: 226 - DNA, 20 bp, Homo sapiens
cctggcagatttgcatgaca

SEQ ID NO: 227 - DNA, 23 bp, Homo sapiens
caagtggaaggaagagcaatcaa

SEQ ID NO: 228 - DNA, 21 bp, Homo sapiens
ggcgtcttcattcgctacaaa

SEQ ID NO: 229 - DNA, 25 bp, Homo sapiens
acagggaaaccttcacaatgtagtc

SEQ ID NO: 230 - DNA, 21 bp, Homo sapiens
ccatcagcacgtttggagtgt

SEQ ID NO: 231 - DNA, 20 bp, Homo sapiens
ctagcccaccagcatccatt

SEQ ID NO: 232 - DNA, 23 bp, Homo sapiens
cctcaacagcaacatctcatcag

SEQ ID NO: 233 - DNA, 21 bp, Homo sapiens
cccacagcttctggttttgac

SEQ ID NO: 234 - DNA, 22 bp, Homo sapiens
gctcaggaggccagactattca

SEQ ID NO: 235 - DNA, 21 bp, Homo sapiens
tggagtgcagtggtgtgatca

SEQ ID NO: 236 - DNA, 22 bp, Homo sapiens
cctttggaggtgatgtcattga

SEQ ID NO: 237 - DNA, 22 bp, Homo sapiens
tgcgctcttggagtttcctact

SEQ ID NO: 238 - DNA, 21 bp, Homo sapiens
gggaacagattgctccatggt

SEQ ID NO: 239 - DNA, 22 bp, Homo sapiens
tgcattgacgctaggaagaaag

SEQ ID NO: 240 - DNA, 22 bp, Homo sapiens
tgtgggaccagaggaagaaatg

SEQ ID NO: 241 - DNA, 24 bp, Homo sapiens
caacccatagttttgctgagtcat

SEQ ID NO: 242 - DNA, 20 bp, Homo sapiens
gaagggtggaggtggatgaa

SEQ ID NO: 243 - DNA, 22 bp, Homo sapiens
cacgcaagtccctaagctgtaa

SEQ ID NO: 244 - DNA, 23 bp, Homo sapiens
gagtacagcaacagtggctccat

SEQ ID NO: 245 - DNA, 21 bp, Homo sapiens
cagctagcatccatcccatca

SEQ ID NO: 246 - DNA, 22 bp, Homo sapiens
gaaggtcacaccctctggtctt

SEQ ID NO: 247 - DNA, 21 bp, Homo sapiens
tggatgccgtcaattcagatt

SEQ ID NO: 248 - DNA, 21 bp, Homo sapiens
gcctcgtccttcaccatttgt

SEQ ID NO: 249 - DNA, 23 bp, Homo sapiens
ggatttccagcctcatcttaaca

SEQ ID NO: 250 - DNA, 25 bp, Homo sapiens
cccaaccaaacaaagacagttactc

SEQ ID NO: 251 - DNA, 21 bp, Homo sapiens
ccttttcctttcctgcacaca

SEQ ID NO: 252 - DNA, 21 bp, Homo sapiens
ccattgctcagtggatgttca

SEQ ID NO: 253 - DNA, 21 bp, Homo sapiens
gggaggctgaggaatttgagt

SEQ ID NO: 254 - DNA, 23 bp, Homo sapiens
gggctcttagtatcggaggattg

SEQ ID NO: 255 - DNA, 23 bp, Homo sapiens
cccaacacaggagagactaagga

SEQ ID NO: 256 - DNA, 23 bp, Homo sapiens
ctgattgtgcacctgtggttaaa

SEQ ID NO: 257 - DNA, 23 bp, Homo sapiens
gagggcagatgctgtctaaacat

SEQ ID NO: 258 - DNA, 21 bp, Homo sapiens
gcctagccttgtgtgcaattc

SEQ ID NO: 259 - DNA, 21 bp, Homo sapiens
accctaggatcccagaaagca

SEQ ID NO: 260 - DNA, 23 bp, Homo sapiens
ggtggaggataagcaagagcata

SEQ ID NO: 261 - DNA, 23 bp, Homo sapiens
catcttggtcttctggctcattt

SEQ ID NO: 262 - DNA, 21 bp, Homo sapiens
catgattgagggcttggtgtt

SEQ ID NO: 263 - DNA, 22 bp, Homo sapiens
ccagtcataagcaagcctgtca

SEQ ID NO: 264 - DNA, 455 bp, Homo sapiens
ctacaagtggtcaaagatctacctgtaactgtctagatatttgcctctaaataatgagacaatgcgaatgcaaagagccagtatgattaagaatatgaccattttcagaaaaagcatattgactctcttgggtcagatatggtggctcacacctataatcccagtactatgggaggctgaggctggagaatctcttgaggccaggagtttgagaacagcctgggcaacatggtgaaaccctgcctctctacaaaagtaaattaaataaatgaaaattttcacacagattaagagtttatttaaaaatatctttctcataaatactagttaatttcttttcacttatgaaattttttatagtaatttatacttttggttcaggcaagctgtgttcattttgatttaaagtaattcctataggtgttttgacttttctagactataagacctgtgta

SEQ ID NO: 265 - DNA, 912 bp, Homo sapiens
gatctatccttttaactcttaaaatggtccttgatgtcgatattcttaacactctttgatgggtaagaaaattaagactatcaaaggtaacagaaaagaagtaaatggcaactaaagcatggaaagtgagttttataaagaaagtaaaaaaaaaaataacaagtgcaaatatccatacttcaattgtgactcaaagccaacatgactctgtctacatttcagcatctcacttaagattcttgaagagggtaagctgatactcaagaagaattagtctttatatttagcctctttttctcattgttatcacaaattggttttcttttagttaccatcagaaaataatatttttttaaatgtgaagattcccaaatattataacagacaaataacagataattataatttaaaaaatccatcgagagattgtggtattaaatttgctacagagtggcctttagttaaatctctcatgccttcacaaagccagaattattctctataaaagttattcttaattggcttcttaatcaggaatttttaaattgtacatagttgtgtatactttctatttattcagaggaaaatgcaattaagtttttagaccatttgctttacttctgtccccagataaaaatgtaaattgtttagtcactatccacaactatgaatagattattttaaaaaataaacctgacttaattttaagcaaaaagcaagtcttgagtattttgccaatctactttttttaatttgtaatattgtttaatctactgtcacttgtaagtacttcggtgtaattgtaaaatggctcccaagattttggaaatgagggaaatagaattccagctgagagttcattaaatcattacttaatgagttcatgcaaagccccagaaggagataaaatag

SEQ ID NO: 266 - DNA, 1412 bp, Homo sapiens
  misc_feature (1329)..(1329): wherein “n” equals A, C, G, or T.
gatattattaattcttaaaactgaatcctccatagaatcctaaaatttgtcatggactataacatatatcacatttaattttctcaaaggtcttgtagggtacataaaggagggactgcccctgattttacattaaattgcttattaggtgagagaatttttgtgggaccagaggaagaaatgcgttatatgtctcagtgctcttggcataattgtgtatgcagagtacatcttattttggtgatgtttttgtatgaaagacttttgagctcattgttatgactcagcaaaactatgggttgtattagttaatctgactcattccttaatggacataattattttacaagggtaaatactgtttctccatcaagactggttaaactattccatgtataaaggtcagctacatcagttttggttagaggtgtggacatttaaaataggtggattaaaataaagaatattccaaagataattgcccaaaatatccaaaccagtatttgcagctcaagtgtatacctgccgtgatggttatctgaacatcattttgtacctttgtttgcatttatttatgttttattttatattaaacatatgcagcccatgtaagtttcaaaacagttaataattctatcttctcaatgaaaaaaaaatctgattcctagagctctaccctttcatttttactcattatggcttctctcttatgaaggattttctgtaatcaaatatttacgtgagacttgtataaaaattattcttcgtagacaaaaaatatagatattggtagaatatggccaaggaaatgttattttgaatgtaatcctgaaacatctgaatatgcttgtgtttaaatgtattattattttaatttttaggaaaagcccgatggctccccagtatttattgccttcagatcctctacaaagaaaagtgtgcagtacgacgatgtaccagaatacaaagacagattgaacctctcagaaaactacactttgtctatcagtaatgcaaggatcagtgatgaaaagagatttgtgtgcatgctagtaactgaggacaacgtgtttgaggcacctacaatagtcaaggtgttcaagcaaccatctaaacctgaaattgtaagcaaagcactgtttctcgaaacagagcagctaaaaaagttgggtgactgcatttcagaagacagttatccagatggcaatatcacatggtacaggaatggaaaagtgctacatccccttgaaggagcggtggtcataatttttaaaaaggaaatggacccagtgactcagctctataccatgacttccaccctggagtacnagacaaccaaggctgacatacaaatgccattcacctgctcggtgacatattatggaccatctggccagaaaacaattcattct

SEQ ID NO: 267 - DNA, 1925 bp, Homo sapiens
tttcacgattagttttagcttaaaaatgtcagctctgggcttaatgaagaaaatatggatatactttatgtcaatgcattaaagtgaatggccataaaagcttatcccagagacaaaacaattcagatataagagaagtgggagagtggaaggtttatctaatcttctgtaggcaactccacagctacaaccagaaggccattttgttacaggcctgaaagccccgttttctttttattcttctttgaaacctttagaaggaacaaagtattggctactttttaccgctgatgtcagtgttaagaatcttgtgataacatagatttactctccctgctgaaaatcactatgtggctcatcagtaacacaactagacatgatgacttaatgcaaaggaagtcctatgtaaatgagcaatgaaattgcaactgtgtataaggaacaaaatagaatatgaaactccagaatcttttgttttcatttctgtttctcccaaggctctatcattcaaaactccagaatctttcagcatgcaattgtctcctgatatcagcccctctcttgttttgttttcttttttttttttttaatcacagtgagccacaacctaggagtcttttagtggtttctacttggtttgctctgcagcctaccagcagatttcctacattccggtcttgttcccctctagcccattctccacactgcagtcataatgaaatttctttcttttttgggggggatggagtctcactctgtcacccaggttggagtgcagtggcatgatctcggctcactgccacctttgcctcctgggttcaagcgattctcatgcctcagcctcccaagtagctgagattatacgcacctgctaccacgcccagctaatcttgtatttttagcagagacagggttttgccacgttggccaggctggtctctaactcctgacctcaagtgatcgcccaccttggctttctctctctttttttttttttggattttgagacaggatctggcctcgttgcctaggctggagtgcagtggcacgatatcagctcactgcaacctctgcctcctgagctcaagccatcctcccacctcagcctcctgagcagctgggactgcaggtgtacaccaccacgcctggctaatttttgtattttattttatttatttttttggtagagacggggttttgctgtgttgcccaagcttgtcttgaactcctggggctcaagcgatctacccatcttggcctcccaaggtgctgggatgacaggcatgagccaccacagctggcctataatgaaatttccaacttacagctattgccattatccaaagcccagaatccctgatttccttccatagcccttcatggcctgaccagtgcctgactctccagcctcacacttcatattctctctgtactgctctgcactgtagcctcattgagttgctttcacgtctttaagtgttgtgttctattttttgtggaattcagcatatgttatgcccttgacctaaggcctctcctttcttttccttctctggggtgctgcctcatccttctggtcttcaaaaccgtttccctgggaaaacatctttgactcagcaggcagggatcatgcccctgctgtgtctgtgcataactttctgtggctacttctgtcttggtctgtgatgtactttataataattttggtctttcctccagtgtcacaatactggaagtctgtttctttttctctgtgttgtatccttagtgcctgaaaggtaggaggttctcaataaatatttgttaaataatcaagtaaatggagtctggtggaaaagagaaaaaataagtgtagaatgtgtgtgcaagaaaggaggggtagggggatgaaaaagataacaaaagcacataacaaaacaacaaaa

SEQ ID NO: 268 - DNA, 2632 bp, Homo sapiens
ttgcaaatatgttttgaaatatatttttggcttttgaattttcccttgagaattgtgtagagaagaatatacaaatcaaagaggatttaatatattattcattgcatatctttccttctgagattttgtttgttttaaatctttggaaagtatgttactcatttcagtatttccactgactttcactggtagatggttcttactaaattaatttcctgccatactatgttaaaaattttattctcaatagatattagccccatattgttttttgagacagggtcttgctctattacccatgctggagtgcagtagtagaatcaaaaatttttagagtcagtatactcatgtaagctaacataaatgagaaagagagagagcgagagaaagaaaggaaaggaggaagtgggaaggggaaaagaggggagaggagtggagggaggggaggggaggggagggagatactcttactcagaaattttctttctttgaaaatcccttatgacatttctaagaagaagcaagaatagtgtgacctttgcaaattaccttaaagacaaagaggagaagaaagagccaagctaatacatgaagagggaaaacaaccagaaaaaatgacatttcagacacaatcatggacagaaatcctacaagtcagtaggggccacctttacctgccagggggaccacaaaaataggggatttctgtcaagaaggcaggaatgttcagcagaacacagcttctgaatcatctgactctctcagaaccaagacaaaacagttcaaatgcctacaagccacaggacccaggaaataccgcagagtggacactttccccctctacataaaagaacctatttcttttctatgcatcagcttctccagtccatctttcattaaaaggacttgccatggaatgaaaactcatatttcaggactaagatggacaacaggccttctccagctcttctctgaaaagtgagcttttcggtagagaacgagcttccttcacaagaagggcactcccgctgggtgtgagccaaacgcacatgcacgacacttgcgcagctaagaatacgcacagtggggaaaaggcacagaagcagcccccgtcctgcccgagtgccacatccctttctgggctttcattcccccacccccaccgcctgcaaaatgaaagaaagattgcaataaacaaggtgtaagtctcaaacctgctcttcacctggagcttgtaatcaggtgtcaggctcccatccacccacaaggaacagagagattttggtgttgaagcttcaacctgccctgcgagccaatctttatttcaaagtactttgtgctgtaagctaacgggaaaaaatgatcaaatgcctcaaatctcccgtaagcagggactgtgcctggggggaaaggtgctcaccaaggtgggggcacatcgggtgtctcctggtgctttctgctggcactaacattctaaaacatgaagcattaagtacagcaacatggatcttccttttttaacatggaaaatacgttttcatagagcaggagggaaaagaactctctaaaaaacagagctgaataggcttagcaagaaaagaaattcaggagatggagaggaggagctctaaaacatccacaaaaaaataaaccatttcatagcaatgctgaccattttaattgattctcgacgacagaagaacacaagaaaaggtagatgatgtaatgcgatggctgctgaaggcaaaagtcacaaaacaaatttagcccttcgaataccacagtagccacgggtcaatataaaaagcttcaacggtcaggagcaaaactggggtgaaggggctactcccccatacatgtaatttgtccaagccctgccatagccaccacctccctggatcctcaaagcaaccctattatgcaagacatgctgatccaggtgcatctgacgattcagaaaaccaggaccaagccgtggggcaccgagcctgagctaataagcagcagagtcgaccctggcacgaaggtctcccagctccatgaagatgcatcatcaagaaggttgggcctcaaattctttccattacacttcatgtttctccctggattatctccataaaggagaaaaacaatacccagaacacaattccaactctgagaaattgtctgatcttcctccttgtctctgcccctcaaaaaaaattttaaccaccattgctttatgttactaatctttttgatggtcctggaaagaactgattttaatttctatttattaatgaatttttgtttttacagttttaactcatgttacctaatcatagcataagaggactgttgcacagtgctcctgcatagagtacagcaacagtggctccatgcatgttacctgctgatgggatggatgctagctgagtgtttgagtagactaatcatgatagatatatttcctgttgtgtgccagacactgtttaggaactgatgatacagaaatatgccttcaggtacctgacaccctcgtggggaagcagacagccatcaattgtgtgatgtaatgtgtcactgtcacgaaaaaaagaagactgggaaaggggacagaggatgagggagttgctagttcatatgtcagtca

SEQ ID NO: 269 - DNA, 1935 bp, Homo sapiens
ttttttgagacagggtcttgctctattacccatgctggagtgcagtagtagaatcaaaaatttttagagtcagtatactcatgtaagctaacataaatgagaaagagagagagcgagagaaagaaaggaaaggaggaagtgggaaggggaaaagaggggagaggagtggagggaggggaggggaggggagggagatactcttactcagaaattttctttctttgaaaatcccttatgacatttctaagaagaagcaagaatagtgtgacctttgcaaattaccttaaagacaaagaggagaagaaagagccaagctaatacatgaagagggaaaacaaccagaaaaaatgacatttcagacacaatcatggacagaaatcctacaagtcagtaggggccacctttacctgccagggggaccacaaaaataggggatttctgtcaagaaggcaggaatgttcagcagaacacagcttctgaatcatctgactctctcagaaccaagacaaaacagttcaaatgcctacaagccacaggacccaggaaataccgcagagtggacactttccccctctacataaaagaacctatttcttttctatgcatcagcttctccagtccatctttcattaaaaggacttgccatggaatgaaaactcatatttcaggactaagatggacaacaggccttctccagctcttctctgaaaagtgagcttttcggtagagaacgagcttccttcacaagaagggcactcccgctgggtgtgagccaaacgcacatgcacgacacttgcgcagctaagaatacgcacagtggggaaaaggcacagaagcagcccccgtcctgcccgagtgccacatccctttctgggctttcattcccccacccccaccgcctgcaaaatgaaagaaagattgcaataaacaaggtgtaagtctcaaacctgctcttcacctggagcttgtaatcaggtgtcaggctcccatccacccacaaggaacagagagattttggtgttgaagcttcaacctgccctgcgagccaatctttatttcaaagtactttgtgctgtaagctaacgggaaaaaatgatcaaatgcctcaaatctcccgtaagcagggactgtgcctggggggaaaggtgctcaccaaggtgggggcacatcgggtgtctcctggtgctttctgctggcactaacattctaaaacatgaagcattaagtacagcaacatggatcttccttttttaacatggaaaatacgttttcatagagcaggagggaaaagaactctctaaaaaacagagctgaataggcttagcaagaaaagaaattcaggagatggagaggaggagctctaaaacatccacaaaaaaataaaccatttcatagcaatgctgaccattttaattgattctcgacgacagaagaacacaagaaaaggtagatgatgtaatgcgatggctgctgaaggcaaaagtcacaaaacaaatttagcccttcgaataccacagtagccacgggtcaatataaaaagcttcaacggtcaggagcaaaactggggtgaaggggctactcccccatacatgtaatttgtccaagccctgccatagccaccacctccctggatcctcaaagcaaccctattatgcaagacatgctgatccaggtgcatctgacgattcagaaaaccaggaccaagccgtggggcaccgagcctgagctaataagcagcagagtcgaccctggcacgaaggtctcccagctccatgaagatgcatcatcaagaaggttgggcctcaaattctttccattacacttcatgtttctccctggattatctccataaaggagaaaaacaatacccagaacacaattccaactctgagaaattgtctgatcttcctccttgtctctgcccctcaaaaaaaa

SEQ ID NO: 270 - DNA, 1302 bp, Homo sapiens
cttgttggcactgaggtaccggtttggaattcccgagcgtcgacggggggaaaaataagaggaatgaatattttaagctttgctatataattaaaatattcttagaagtctggagtctgtgaaggtcacaccctctggtcttctcccagcccatagggtataaataatctgaattgacggcatccagggatctcagaaattattagtacatcccacagtgaattaccaccttactaaaatattcatgggtatatactatggatttgttttatcctatttagtcttaaaaactataaagaaatctgcaggcttattaacatattactcagaatcatattgtctccaaagcacaaactgaatcagttacaagatattggactagagatcatggcaaatcagaggtacataagacctagttccgttgtggagctaaacaaactgcagagacctaaagggaagccttgcaccacactctaggtttggagctcaggttttgagtggtgtcagcactccagaacacatgggatccccgggaggtggaaattgagccgtctttggagaatcagctaatgagacagatgcatgttaaatgtctgttgtggcccaggcactctgctaggcagaggggtgaaccagaagaatgagattcatggggccaaagaatttgccttctggtgtaagaaaagatggaggcagcttggcagaaaaaaaaaaaaggtaaaagatagaaatgaaatacagatgtgaggcaccgtatccaggctgtatggagtctttctaatcaggacataggcagacagtcctagcccagctttatgccttatgagacgcaacaacgttgaacagtccattgtttgagggaccagaggttttaccagatggatgataactagcatctgtggaacattatttgtgaaatatagaaatcagaaattcccagcgtagcactgtcccaaggggaacataatttgacctgcatatttgctggtccatttttagtagtcacattaaaaaagaaaaatgacacaggtgaaattaatttgaatatattttcttaattcagtatgcttaaatattatttaagtatgtactcaatataagcaattgttaatgaaatattttactctttttgaactatgtgtttgaaaccccggatgtattttttttttatcttcaccacacatttcaatttgggttggtcacatttcaagtgctcaggagtcacatgcagctaggggttacctattggacaggcaggcagatcttgagagctccaaagaactgtgtgtcattatattgtgg

SEQ ID NO: 271 - DNA, 581 bp, Homo sapiens
tggctgaaaactttaaaagctcaggttagttcagatagattcagtgtgagctgaaagccagccccctggccctgcggtgactttttccaaaagataaatgagtgaggccaggagtgtcatgcagacgggctttgggccggctatgggtgttggcattcttgttttgaaacccccttccacatctgctcaggggtcacaatcttaagtgctgaaggggtgcagctgatgaatgagaaaagcagacagtgtggagcctggggagctggtccttgcctcgtccttcaccatttattgccctgtgggagtgcaaagttagtgtttccagatcttctgattgttaagagaggctggaaatccgtatttttcaagaggattgagttgccaactcattgaaatcttctccaagccccttgcgagtcagcattggttagcatgtctcgaacacatggtagctcaaacacacacggtagcttgccatggtggcaatttcaaattgcattcattgatttcaaaagaccatcaatttcaaattgcattcatcttttgagttgcgaaataataaacacgaaaaaaaaaaaaaa

SEQ ID NO: 272 - DNA, 460 bp, Homo sapiens
agaatgatggatcattggtgataaatacacaaaaacccaaccaaacaaagacagttactccaggaataacaaaaatgtgtgcaggaaaggaaaaggattccaagtacacaaggaactcagctgcccctatagcacttagaaagtcatgataaagtcaacagtgaacacagagttaaaactctgtggggacaggggaaaatatttgtcatgggaagtgaggggatatttgagtaagtgaatgttggatctttatcttccataatggcaggttcataacaatggctacaaactatagcagttaaaagaattagccggggcccggtgtggtggcttacacccataatcttagcactctaggaggccaaggcaggcagatcactcgaggtccggagttcaagaccagcctggccaacatggtgaaacctgtctctactaaaaatacaaaaaaaaaaaagaccag

SEQ ID NO: 273 - DNA, 1335 bp, Homo sapiens
cctgtgacatttctttcaggaagtcttacaccttacttgactccacagtctaattagatgttacctccttgggatcccacagtagtgtatgtgcctctttcacagcaatgctgtctatactgacccctgatttgcatgtatacctttcctcaggatatgaactttctgaatgtagtgaccataccctatttattttgatatcgccaaattctagcttgtgcttgacatagagttgtttcccagctaaatgttgaataaaaaaccaaactgaaaaaacataggtagcattatgtgtaatatttactatataggttctgatttaaatgctttacatatattaacttatttaatcatcatagcaacactatggggtaagtactattattcctgcctccattttacaggtgaggaaactgaggcttgcagagattaaataactctcccaaagccacacagctagtaagtggtggagctaggattcaaacccaggtagtgtggcttcacagttagtgctttaaccactacattgtatgtgtgcctctaggagggtcactgagatttatgataaacatatatattgattgtccaagaaaaggtgaagaaacattaaccataagtcacaattccatgaacacatttaaaagtaattagtaaatgtgcagagacactgttaggggagtggatgttactactgtcatttatgaaggatttgctagagatggtagatttcacctgttgtgaattggaggaggagcatggctggcaattcgaaaggaggtaatctctctggggtacaatggagtagaaaacttagggacagaaggaatatacgaatggagaaattcgatttgcccaatctttattgctcacctattaaagtgctaaacaagctgatggtgattcctgttctcagaagcctgtgttctagcaggttataagaagatgagtctggttaaagagaagagcagggaagtggcttagattatggcataaactgaagttgaaactcagaatgaaaagtaggagtttgctgaggggaaagcaatatataaagtgatttgtgctataggacataagacagattatagataagagaactcagaaatagtaaggacagtggtaaaaagttaaaggatcctccctttccccagttaaccaggagaccaaataagggacttggtggtaggagtggtaggagcaggatcaatcacttatttattaagcacctgcacatgattcaaagaaggataagacggcccctacccttaaggagtttatgttctttctagttgtgaatagagaaagcatatgcaaaaaaaaaaaaaaaaaaaggac

SEQ ID NO: 274 - DNA, 1466 bp, Homo sapiens
gccaagttctgcaaaagatccataccagttcactcgtgtgcgactgtggacaggtaagtcactttggtctctatgaacctcagttttccagatctttgaaatgagcacttggatgcctatccttgcttccacacaagtgtttttttttttttttttttttttttgtgagaatcgattgaaatagtatatgaagtggtttgaaaataggtacaaactattgacatttcaatgtcagagagtgatacctgtagtagtataggcaaaggtccaaccccatcgaaaggcttaaacatttaccttttctgaaaaactattgaaatataaagagagtccccagtcacaggggcaacttctgtaaccaaatccagatctgaggaaactcctgtaaccccatttggggtttctttctaagccaatagggttacaggttggtacagtgacacattgagaatggggctacaaatacttttcccaccatctaggatgaaatacacgaaatcctgttgaaatcttggtttttatgcctttgctcatcagaataaacgtaaatgctgaaaaacaaataacctcctgatccactgtcttgcctcctggtgagaaatgattctatcccctgtttattgggaaatttccaaagttgttcatcacttaaatgccgtattcaaagggaacatggaaggatgaagcggagaaagtgccttcgagacattcacacatttctctggactcagtctgttaacatatcagggagcttgtcagatcacacctttttgccttggaaatcctacagatttcctgtacgccttcatatctgattcttccctaaaacctttgggtatgatttcctccctggtcttgataatgtcctgcagtctgtgttttataattattctttgtatttattgaatctagactttaagttattcagagatcagaccagaaccttagagtttctaaactgtatgtggatattaaataatattaataatgaaagagctaccaaaatagtctatattgtgtgaacaatctcttgggatattagacgtgtttaaagaccagtgttgctgctatttttaatattttggttaatttaagtgaaatgtacatattttaatttgaagatttatcttgcccatcagaatgtgaagatatacttgcatatattttgacatatttcatggaaaataaaaatgataatccactttgtgagtgtaagtgaatgtattcatatgtatgttattataaatgatttttgtttgcactgatgatgaaatgagagttttgggggctttttatacatttatatcgactggtctctaaatctcctattttgttttcttatcatttttgaaatacagttcccattacatgagttttaaatagattggtgtttcattttgtattatgctactactagatgttgattctctggtattgtaaaataaaatgtgctccaaaaacccaaa

SEQ ID NO: 275 - DNA, 2539 bp, Homo sapiens
tccggtgggttcttggtcttgctgacttcaagaatgaagctgcagaccttcgcagtgagtgttacagctcttaaagatgatgtgtctggagtttgttccttcagatgtgtctggagtttggtgggttcatagtctcgctgactttaagaatgaagccacggacgttcgcatgttacagctcttaaaggtggtgtggacccaaagagtgagcagcagcaagatttattgtgaagaacaaaagaacaaagcttccacagcgtggaaggggacccaagtgggttgctgctgctggctggggtggccagtttttattccctgatttgtccccacccacgtcctactgattggtccatcttacagggtgctgattggtcagttttacagagtgttgattggtgcgtttacaaacctttagctagacacagagcgctgactgatgcctttttacagagttctgactggtgcatttacaatcctttagctagacgtagagcgctgattggtatgtttttacagagtgctgagtggtgcgtttacaatcctccagctagacacagtgctgactggtgagtttttacagagtgctgattggtgtgtttacaatcctctagctagacacagagcgctgattggtgtgtttttacagagtgctgattggtgcgtttacagtcctctagctagacacagagtgctgattggtgtgtttttacagagtgctgattggtgcatttacaatcctttagctagacacacagcgctgattggtgcgtttctacagagtgctgactggtgcatttacaatcctctagctagacagaaaagttctccaagtccccactcaacccaggaagtccagctggtttcacctctcactagcactttgggaggctaaggcaggaggcttacttgagcccaggagtttgggaccagcctgggagacatagtgagaccctatctctttaaaataaaattagccaggtgtggtggtggtgtgcatctgtagtcccagctacactagtggctgaaacaaaaggattgcttgagcctaggtggtcaaggctgcagattttgagctgtgatcatgccatttcactcaagcctcggtgacaaggcaaaacactgtctatataataataataataataaataatccatctcacatattcttgtgaaaacgaaaggaatgtatgaataaatgttttgtaagttgcacagcattatgagtttaagttgaggaatttaggagtgtatatatttttatatcctgcctggttccaaagaggtttacagtggctcagatctaatgtgttatttttcctccatcaccaggatacttggtggttacttagtacaggtttatgaaattaaattgaatgcaagtcttcatgaagaagaaagattgggctgaaagtttagctttttgctctagctgcttctggtttttgagttatatcattagaaataccagataacaagtgaaaagtcattcagctcctttcatttaaaatcttgacagttttctttttttaaggtcaaccagcaaatgatatcctgcctcttgaaaacttaatcattttatctgacaggagttagattaggtgtctccagagcatttgcttatacttaaagtgccagaagaggttctcagtcctaacaaaacaaacaaaaaaacccactttctcaaagtttctctctttagtcactttgtattagattcatccattttaaaaatctttgctttagaagcattgttaatgtttttgtccatttcactagagtccctgaggaacatcatcttgggtttaacagtattaattgaccacccactatgtagccagctatgtgctaaatgctgaaaaaaataagaatacgttgcaaccctgtcattgaggaggcatattagttagatttctgctgtgacaatattgcatatcacacaatcccaaaatctcagtggcttacaattgcaaacatttatttcatgttcatgggtgtgcaggttggctgtggttcagctgtgtcactaggctgaacttactcaataagccacataacttcgagtcaggttccagtccattgtatgtgttattttcaaaatctaggctaaaggaggaacagtcatgtgggtcctactcttcctatggtggaaggtttaagcttaaaagggttggtgattattatgccttaaagtcttagctcaacagtggtacagtgcaatgtcttccatttctgttaccaaagcgagtcacaggaccaagcccaaagtcaatgacattagtcaatgtactcttcctggtaggaggtcttgcaaaggtcatgttgcaaagagtgaggatatataatattactagagggaggaggtgcctaattgggaagaataatccagtctaggctgcgcacagtggctgaagcttggaaacccagtgctttgggaggctgaagtgggaggagatcgcttaaggccagaagttcgagaccagcctgggcaacctagttgagaccctagcccaaaaaaaaaaaaaaaaaaaaaaaaaaaaag

SEQ ID NO: 276 - DNA, 1563 bp, Homo sapiens
tctgtcatcgaggctggagtgcaatggtgcaatcttggctcactgcaacctccaccttccagactcaagtgattatcgtgcctcagccttctgagtagctgggatcacaggcgtgtgccaccattcccggctaatttttgtatttttagtagagacaggtttttgccacgttggccagtctggtctcaagctcctgacctcaagtgatccacatgccttggttgaccaaattgctgggattacaggcatgagccaatatgaccagctcaaacatcttctttttaaatgtcagaagcatgtatagtgattatttcttattttttcccccttgatccatctcaccagatgtttgttgattttataagaattttcaaactaccagcttctggctttgttgaacttggatttctgtttcactaattttctttctcctgtctttgtacttactttgttgctctttttctaagttttaaagatggatgccaatctcaggcttcttttcgtgtgtgtatgtgcgtatgtccataaattctcttctaattacagtgtaagccgcatcccacaagttttgatagtcacagaactgtatcgtcacactattttttaatttcagtaagttcttcactgatccctgtgtaatttagaaatgtttcataatttccctacattggaggggaagatagttttgtttttattattaatttctagctgtattgagctcttgtcagagaatatggtttattttagtcgtttgaaatttaagatctgcttaatggcaaaatgtatggtcagtttttgtaaatgttgccagtaagcttgcgaatcatatgtactctagttttgaaatccattgctcagtggatgttcattaggccaatttgtataatcatgttgtacaaatctattctattcttaactgttttttgttttaaaggtgtggggtcttactatgttgcccgggctggactcaaattcctcagcctcccaagtatctagaactacaggcacgtgcagcttggtttaaaaaaaaaaaaaaaaatcagtgagaagaggatttgttgatctccccgttaggattatgggtttgtctgttcctccttctcagcttatgctgtatatattttggggctgtgttattaggtgcatccaagtgtatagttgttatagttaccatgtgagctcaaccttggatctttacatagagattctctgtatttagtaatgttttgttcttaaaatctgcttccatctaacattaatataaatgtaccagctttattttatatgtatgtttcttggactttgtctttatgtattacaagaaattgtgataaagacctcatttaactggattgtgaaaggactaggccattctgggtcatttacttttctgaaaaatatttttattttcttggtatttaaaaaaaggtttataagacattctaatttatcttagttttcttccttcatttatttaggggtctggtatcttagggatatcattctgaaaattaaacttttctacataggaccatagatacagggtgactagatgactgggct

SEQ ID NO: 277 - DNA, 2683 bp, Homo sapiens
ggaataatgcaggttctgggcagggatggaaagagtgaatgcgctggtacggtaaggtgcctcgcaggcacgtgagggcctctctaatcgttagctattgtcaccgattgtattgttatgacttctaccaccaccactccccctcctcctgggatggtgattccagggccaggcggcaggctataactagcgcccttccaggtggaacccgccagagccccgaggcagcctaggattttctgagatcagacacacttgggccgggttgggaggaactggcaggaaaaggactgaacccttaatgtcaggcggttttgaagcacctggggcaagctatggaaatccccacaggaaggctcacgcagtctcttaggcggctgccctccacctgccacgttctttttgattgactaaaaaacgctgaatgaagaacgaagtcgcgtggaaaccctcgccgcgcgcctgcagcggacagcgcagcccgggaggttcggctgccgacttgcgcccgggggctgcgctgcgagcggccacgcatggcggctggacccgggcggccgcaagggcttggctgggccgcgggaggcgggaggttcttcgtcctcccgagccatctccctgaactgacaagcaggactcccgggtccagggggcacagggcccggggcggtgacccggcggatcgggctgccggaggagcccactgtaaatgccgcaactggccccaaacactgcgttcctggactgcaccagcagctcctggcgcggccgcagagttggtggatattttccaagggggaaaaaaatcttttaaatgccatctgtttactttaaaaatgttgattacttaagaaaaacgaatggatgtctgggcaaaggtatggacgtcacaattattttgaaggcgtcctttttaactttaaacagaccacgccaggaggagactgctgacccagagcgcattacctaaaatctggtacccagagtgcacccttcgccctcgttggagttctctcctctctgccaagctttgctccgtgccagaggtgtgctccattgtacctccgctctgtccctgcagtcaggcaaccaattggagaagagtataaatagtaattaaccagggagagttgtaattcagaaacctagttaaaacaagtcctcaaaaactagagaatatgagagtggggagacattttgaaggcattaagaacaaaaaacgatggggacgaatggttgagtctgaggatcagcatcgtaatctgttagagaacgaggtcgtggctgtgtctgtgagtcgttaatgggtttaatcggttgatacacagcctgctagtggcctaaccagtaacccagggcctggcagatttgcatgacatctcggagtttgattgctcttccttccacttggcaaaaggagacaccatcagccggatcaggaggggtcatggtgagatggaacccaccgaggtggtgtacagagctggcgctgccaatggccagagtggcagcctttctacctccttaaccctgcaaaaatcaaacgtgctagtacgcactgtccatccacactggaactccagttggttttagtctgcgatgatgactcttctgggttgacttttccagttcatcatgcctttctacctccttaaccctgcaaaaatcaaacgtgctagtacgcactgtccatccacactggaactccagttggttttagtctgcgatgatgactcttctgggttgacttttccagttcattatgcagccctcttgaagcaggcctcccaaacttagcagacaccaatgagaacctcacaaagaggctcatcaagcaggctggtgaaactgggtgttacttcctgttccatgggtaccccatagtgtttgggaaacaccgggctgtggttcaggagaatttcacatatgctaagatggagaaagaacctgccctttacatttaggcttgggatgttaatttaaagtttgaatgaccaaaaattaaatctgtaacttttaaagtttctctttgtgattttacttaagtgttggtagatattcttaaattgtaatgacctcagtttgggaattaagttagccaaatattgtgtaattattgtttgttatacaaaaatatgccttagactgtacagcggcagaaactccctctaccacctcggtccccctttccattctgcgttatacaaaataagctgacacgttaatgctgtggcccacattaaacaaagtataccgtacgtgtgtgtgtgtgtatgtggcataataaatggtggtagctaacacttaccgaatgttttccctatgttccaggcactgtttcaagttttacaggattagcaaatttaatcctcattacagttctgtgaagtaggtacttttacaggtgaggaaacacaggcacagagaggttaagcaatttgcccaagatctcacagctgggaagtaccaaagctaatataccaacccaggcagtcctgctccagagatcgttctggaccattctggatcacacttcctcgcttaagtgattgaagcaagatatttatcatatagcatgggtccaaaactgagtttgctttagaagagtttgacagctttctgacatgcctttagtggtctcagcgcagactgcagattttgtcattcacttgaaaagaatatc

SEQ ID NO: 278 - DNA, 769 bp, Homo sapiens
gatcgtgccattgcactccagtctaggccacaacagcaaaactccgtctaaaaataaataaataaataaaactgaatgaatataaacagaaaccacagatgctattacatattaaattgataatataaccattacaggcgtgagccaccaagcctggcctaaaacatttaaaaatgtttattttaaacatacataagacatgcacacataaagatacgcatagcatgattgagggcttggtgttttgtttctgtaacactggatttgaaacgaaactataatgagaatgtatagcagggctgggcgaatgacaggcttgcttatgactggagggtcaagggctattgagtgcaaaagctggatgtaatcagattagctcagtgttttgtttttatagctatgcattttagcgtttaaaccatggtaaagaacagcttttaaaaaaaaatcgcttctcagccttttggctaagctcaagtgtaaaaaaaaaaaaaacagctttaaatctcaagcttttgcccctaatcttttaaaatttcattgaaataattatcagtttactgtttcactgcaccacaaatttagtttcaggtgtatcttgaaactcattgatatgctaataagttttattaaaattgttaaattccttcctatgaatatactttttatacagatgtgacttaagtatttaaatgttttacttattcacaaaataacaaagaatggcaaaaaaaaagcataagctcaagtgtaaaaaaaaaaaaaaagg

SEQ ID NO: 279 - DNA, 2842 bp, Homo sapiens
aataagtacatcagacaacaagtcaagtcaagtctttgcctcatggagctaacattctaagaggagaaacatgcagtaaacaagtaaagaaatgtatgctctattcagggagtagtttgtgctatgaggaaaagcaaaacaggttgaagagatagctatgtggtgggagtgggactatttcgtacagggcactgattgtagacctctgatgagataacatttgacaagagatctgcagggagctatgtgtcatgggggaaggcattggagggttttgtgcaggacagtgatgtgtgatcagatttagtttaaaagaataatttgggctgggcatggtggttcctgcctgtaatcccagcactttgggagggtgaggtgggcgaatcacttgaatctgggagtttgataccagttcgggcaacatggcgaaatcccgtctctacaaaaaatacaaaaattagccagtgtggtggcacgcgcctgcagtcccagctacttgggaggctgaggtgggagaattgcttggatctgggaggtggaggttgcagtgaactcagattgcgccactgcactccagcctgagattgtgccactgcactccagccactgcactccaggaagaccctctcagaaaaaaaaaaaaaagaatttggccgttatgtggaggactggaattgagaagggcaagagcgaggtagaagagtggtctagggagaacagttaggggctattgcaattatccagcaagagatcttggaccaggatggcagcagtggaggtggtaaaatgtggttggatgaagcgtacgctttgaaggtatcaacaggaccagctgatggaagggagtcaacaggactagctgatggctgtaaactggggggtcactagctatcagatggcatttacttaaagccatggaagtaggtgagctcccttatggagagggaataggaaggaggtagaccattctatcaaaatgctctttctacagggcacttctcactgagatattatttatctgggatttatattatttattcaatttgttttgtgtttggttctattagaaaagctccataggggccgggcacgttggcttttgcctgtaatcccaacactttggaaggccgaggcaggcggattacctggggtcaggagtttgagaccagcctggccaacatggtgaaactctgtctctactaaaaacacaaaaattagccgggcgtggtggtgcgcctgtaatcccagatgctgaggaggagaatcgcttgaacccgggaggtggaggttgcagtgagccgagatcgcgccactgcactccagcctgggcaacaagagcgaaactccctctcaaaaacaaacaaacaaacaaacaaacaaacaaaaaacaaaaaaaagaaagaaagaaagaaaagggccaggtgtggtggctcacacctgtaatcccagcactttgggaggctgaggcaggcggatcacgaggtcaggagatcgagaccatcctcaccaacacggtgaaaccccgtctctactaaaaatacaaaaattagtcgggtgtggtggcgggcgcctgtagtcccaggtactccgcaggctgaggcaggagaatcgcttgaacccgggaggcggaggttgtagtgagccgagattgagccactgcactccagcctgggtgacaaagtgagactccatctcaaaagaaaaaagctccataggagaaggaaccttgtctcttcaccacataaactgtgtttggattcgcaatcgagttgggaaaaaaaaatcagtctggaagagccacaccaaaccgctaacagctactgtctctgggaatagaacaaggagtttggttggcgcgatataccgcccctgaacctctagccacaataaggcttaattaatgaccggacgacttgaaagcgccttccactgtttatctcttaaatctgcaacgaaatgcaacaaaaacgcaagaaataaacaatagaagccagtcttactgcacactgcagaagccaataaaccccaaatgtagctcaaaacaaggtgtcacgcaaacttctgatttttttttgttttacactgaatctctgtcactctgactagagggcagtggcgcgatctcggatcactacaacctccgtcttctagagtcaagagactctcccgccccaggagtctctgccactctgactagagggcagtggcgcgatctcggatcactacaacctccgtcttctagagtcaagagactctcccgccccagcttctccaataggtgggattgcaaacaggcaccaccacgcccgaataagtttgtgaacatttggtacaaataacaaatgaccaagttccttggttgtagttgcctaacttttaatacttaaaaatgtagcctcaggaaataagaggcctcaaaaaattgaataaaaactcacaactttctctccacggaaatctttagtaaaaggcgaaagatttatgcgctttgaagagaaacccgagtatattcgtgacttccgcttcgaacctcgcagggagaactaacacttaacacacttatggttgttggatgcctgcgtggtacgcactccctatatgtagtttatgcacacagatgcgtgtaagaggcatcatgctctaaaacagtgcagaaatgcgcgcacagagggagtgcaagcatcttgagggtatcttttcgtggtgcaccatgggtatgcaaatcacaggcggctccgggctgttccgcgccaccggggaagccatgggtcactgatctcctttgctctcctatgctcctctctgctggtcctcctgggacccgcaccccgtgggcggcgcc

SEQ ID NO: 280 - DNA, 3329 bp, Homo sapiens
ccctgcgctgtcgggcggggaggtcggaaaccccctggcgagaccacgggcggacgcttcccgaagagctgcctgggctgcagccgcggaagctgcgttctggggagcggggagcgtgctccggcgccttcgggccgctgctggaagccggaaccgagcccgggccgctgcccctcaccggacgccgcgcgccaccggccctccgcggggcaggggctgctgcgagctcgccgggcgccctttagacagtcgtccttgtctactccactaccaaatgttgaagttcttcaagaatcagtcctttggaggtgatgtcattgaaaatgatgagtaggaaactccaagagcgcatttctccacaaaaccagtgaatacattggcacaaattgtcagaatcaattttatataaattctggaaattagtcaaaggtttatagtaaccaaggaaacatctttttaaaaagatggctgagtggaccttcttttcaaagaattatggaggcttattttagttcccctaacttggaaatctcctgaggaagaaaggtgactacaggcatttgtcaaaaatttgtaaaggcaagtttattagcctctgccatcgggggcaaagaataatagctaaggcaaacaatagacacaccaaaaagcctgggaggaaaagctggaaagtaagatattttggagaataaaggcttttaaaacttccacatattcttgggaatccaaaaggccacatgtacatgcagggtgagcaaatagagaagacttgagaaagccttaaactctcacctctggctaaccatgaggcttgctcaaataggaagtgaaaactaaggtgaatttgttgcttagctgaatgttgaaggtgtgccccaacacttacacagagcctactggtaaagacagagtgttttctttttgtcttggtttcaggcatttaaggaaatctgtttctcttttggatcactagctgcaaattaagctaacagaacaggagctcagctggtcacacacagcaacgaatacagactttataaagttcagaaaagttaccaaacagtggtaaccataacaagtaccaacaatgaactatggggagggaggagaatctgatttccagagttaccacattataatactattcaaaatgtcacatttttagcaaagattacatgacaaggaaaaaccagaaaagtatggcccatacacaggtaaaaaaagaaattaatagaaactacccctgaagaagcacagacttcggatgtacaaaacaaagacttttcatcaactcttttagatatgctagaagagctaaaggaaaccatggacagagaacaaaaaaattaggaaagcaatgtctcatccaatacagaatatcaataaagagattgaaattgtagaaaagaaccaaatagaaattctggagttgaaaagtattataactaaaactgaaaattcactagaggtattcagcagcagactggagaagtcagaagaaagaatcaacaggcttcaagataggtcaattaagattatacagtctgaggagcagaaaggaaaaagaatgaagaaaaatgaacagagcataaaagacctctgggactctatcaagcataccagtatatgcatgaggggagtcccagaaggagaagaaagagagaaagggacataatatttgaagaaataatggtagaaaatgtcccagctttgatgaaatacatgaatctagatattcaagaggctcaaagaaccctaaatagggtaaactcaaaaagacccacaccggaatgcaaaagtgagctgggtgtggtggcacgtgcctgtggtcccagctactcgagaggctaaggcaggaaaatcgcttgaacccaggaggcagagattgcggtgagccgggattgcgccagtgcactccagctgggcgacagagcgagattccatctcgaaaaaaaaaaaaaacaaaaaactattgctgcagtcattcagatggaaatggggaaagaataatattaactgatttcaaaaaggacttgaagatgtgaatcatctattttgctgaagaaatcttaactctttgaaattactttttgttgctgttgtcatactcttaggtgccaaactgcggtaaattttttatcagtgaagtggaagcatgtgttttgttgttttgggaatttttatcaagtatcttcagagaagattatttcctgctttatcttcaaaaactggaaaggaagggtcaaagaaaagacagtagctggccggtcatggtggctcatgcctgtaatcccaacactttgggaggctgaggtgggcagatcacctgaggttgggagttcgaggccagcctgaccaacgtggagaaatgccatctctactaaagatgcaaggattggccgggcatggtggcgcgtgcctgtgatcccagctgctcaggaggctgaggcaggagaatcgcttggacctgggaggtggaggttgcggtgagctgagatcacgccattgcactccagcctgggcaacaagcgaaactctgtctcaaaaaaaaaagaaaagacagtagcttatgttcatgtcaagcacctctcatcacagtctagttccaaggaaaaaattcccagcgttttctacattcggtgctgcgtcatctgaaatcggcacattccatggaggaaggagtcctgctttgttgcatgtatcctagggtttaatgttggtaaatgagtcactctagcatttgtagaaggctccctgagactcctgcagcagtcgaccaagcccaaggacataattgaatctggagagtcctggggccttgttttgaaaaagacttgaaatacacataggaagaaaggcataaaaataaatgttcacttgtctctgctgtgagtatgtgttccaacttttcagtgatggctttgagaattctcaaacttgactggctctaagtgtatctggtggcttttgtatcgtaacctgaaactggcttagtactttttcctaaaagctcaggatttgagaatgaggaccccttcgccaggaaaacatgtatacactcaaaattttgcttgcagttctagggtgtttagacctttctcagatacctgtgcatcttatgggttttgtttttctctttgagacagtctcaccctgttgcccaggctggagtgcagtggcatggtctcagctcattgcagcctccgcctcctgggttcaggtggttctgcctcagccccttgatcggctgggattgcatgcatgtgccaccatgcccggctgatttttgtatttttagtggagatggagacagagtttcaccatgttgg

SEQ ID NO: 281 - DNA, 2182 bp, Homo sapiens
gaaagggccaaatacgactcttaatgatacaacagctaaatataggtctgatgctcattccgtgtggacaacaatagcagccattcccacaaatggctgatttgtaggaagtaaacactacttttgcagaatcttacatgatttcagtagaagggcaaggacatttcagttgggaacagattgctccatggtaatgtgatcactgtgtacccaacaatggctctttcttcctagcgtcaatgcagatgttattttcaccttaactgttatcattgttgtttctaaccacatgaaagtgtatcctttatatatctgaagtaaattcatactagtggtgtaacatctccagccatttaagtgtaaaaacagaaaacgtatgatgtgtttacgtactgttttatactcctaacgcatgaagagaagatccttttattcattgcctatacttttatttctaaactttctgtaacactttatcttatatccagcatagaattaagatttgcttttcgatttaatctgacaatattttttcctctaataagagtcaagtccacttacttttaatgataagttgtgtttggttatattttgattacagtatattatgctatgatttatatgcacatatctgtcttttgctgtcttgtttgtttttattgcttttgttttgatgttgtgatatttggaagagttaaacttttattctgatggctaccttatgtaatttcataaaatcatctctttctttggacagtagctaatgtctctaaactaagaacaatggtattagctgtattctctttcttgtcctccctatgtgatttttcatcccacaatttgatttaatcatattaactttgtttcccctggtgccattaagtatgcttacatttctataaacaatatcctttgactcccaggcattacagatgagcagtcagtaaaatcattctgaggaatactttctctttccttttcttccatttttcttagttgtatcatttctctgatgggtctatttctttaaaacaaagggaggggagtctctcatttacattagtttttttcatagccttttggactttgcaatttctatgttttggaacctatttcttacagtttttctatgctaaactctgtcctggtcagttccagagtgtatgaagaaccaaatcatgtaattgtatgtgacctggctgtagtggaacaaatttgactcttaagtatgcaggctctaattttcctgtctggttttggtaagtattccttacataggttttttctttgaaaatctgggattgagaggttgatgaatgaaaattaaacctttcactttgttgtatataggtttgcaatatttaggtcagagtggagttttaaggtcatgaagggggctgatgacttacaaataatgggctctgattgggcaactactcatctgagttccttccatttgacctaattaagcttgtgaaatttacactaagccatgagctcatctttaaaaagttttgttaaaagattttcagctgttccaaatgggacttattagtggaatgtgttttaaaggatcatatcagatgaatgaaaggtatttgatcctttctttccttaataataaaatgatggtttggaaaaataggctacagtctaaccacagtgctattattaggctttcttgttaaacataggtctaagcctaagtatgtcaatacaacaaatacttactgtttcatttctagtaataaaaaaaaaagtctttctggcataaggatgattttgatctggttattttgaaacatttttgtaaaataaatttacatctataaagaacatttttattcgtaaggaggggtatgtctctgtgcactggaagagagggaggactaaatcactgggaagtcttatgataaagaagccattggcttaaatcagcaaagcaagccatcccttggtttaaggtgtttttcctggccatcctgtcttgactagaactttacctacaccttcctttttggtttaggcaaattatagtatctaaacctgaagtctcagctctgtgtctttgagatataaatgttctaccatgtcttctctggaacctgataactatctatctctttaaaatggaagtctagggagatgactcatcagaaagtctaggaagatgactcatcag

SEQ ID NO: 282 - DNA, 5085 bp, Homo sapiens
ccgtgacctccatgtgggagctccagctctataagtaaacactctgcgcggcgcagacatggcctcttcctatctttgaggcggtgtctgcggcagcgcctcagagtggttccggtcgtctctcctcaagtcggctagtcgggcgcgcgcgctgagagtcgtcgccgcctgtcgggcccggcgtccggtcggtccggtgggcgcgctcgcccgcctgccgctgagggcccgagccgcagggaaagcggcgcgggccgggcggggcgcggcgcccagagctcagggggagacaaaggggaccggttcctctctaggcgccaagatgtggatacaggttcgcaccattgatggctccaagacgtgcaccattgaggacgtgtctcgcaaagccacgattgaggagctgcgcgagcgggtgtgggcgctgttcgacgtgcggcccgaatgccagcgcctcttctaccggggcaagcagttggaaaatggatataccttatttgattatgatgttggactgaatgatataattcagctgctagttcgcccagaccctgatcatcttcctggcacatctacacagattgaggctaaaccctgttctaatagtccacctaaagtaaagaaagctccgagggtaggaccttccaatcagccatctacatcagctcgtgcccgtcttattgatcctggctttggaatatataaggtaaatgaattggtggatgccagagatgtcggccttggtgcttggtttgaagcacacatacatagtgttactagagcttctgatggacagtcacgtggcaaaactccactgaagaatggcagttcttgtaaaaggactaatggaaatataaagcataaatccaaagagaacacaaataaattggacagtgtaccctctacgtctaattcagactgtgttgctgctgatgaagacgttatttaccatatccagtatgatgaatacccagaaagcggtactctagaaatgaatgtcaaggatcttagaccacgagctagaaccattttgaaatggaatgaactaaatgttggtgatgtggtaatggttaattataatgtagaaagtcctggacaaagaggattctggtttgatgcagaaattaccacattgaagacaatctcaaggaccaaaaaagaacttcgtgtgaaaattttcctggggggttctgaaggaacattaaatgactgcaagataatatctgtagatgaaatcttcaagattgagagacctggagcccatcccctttcatttgcagatggaaagtttttaaggcgaaatgaccctgaatgtgacctgtgtggtggagacccagaaaagaaatgtcattcttgctcctgtcgtgtatgtggtgggaaacatgaacccaacatgcagcttctgtgtgatgaatgtaatgtggcttatcatatttactgtctgaatccacctttggataaagtcccagaagaggaatactggtattgtccttcttgtaaaactgattccagtgaagttgtaaaggctggtgaaagactcaagatgagtaaaaagaaagcaaagatgccgtcagctagtactgaaagccgaagagactggggcaggggaatggcttgtgttggtcgtacgagagaatgtactattgtcccttctaatcattatggacccattcctggtattcctgttggatcaacttggagatttagagttcaggtgagcgaagcaggtgttcacagaccccatgttggtggaattcatggtcgaagtaatgatggggcttattctcttgtactggctggtggatttgcggatgaagtcgaccgaggtgatgagttcacatacactggaagcggtggtaaaaatcttgctggtaacaaaagaattggtgcaccttcagctgatcaaacattaacaaacatgaacagggcattggccctaaactgtgatgctccattggatgataaaattggagcagagtctcggaattggagagctggtaagccagtcagagtgatacgcagttttaaagggaggaagatcagcaaatatgctcctgaagaaggcaacagatatgatggcatttataaggtggtgaaatactggccagagatttcatcaagccatggattcttggtttggcgctatcttttaagaagagatgatgttgaacctgctccttggacctctgaaggaatagaacggtcaaggagattatgtctacgtgggttgtgcttgggaaaagttggacctgttaattaaaagtaaaatatttccaaatcaatttggaaatgacttgaagtgtgagggaaagggattcataaaatttaggtataggaggccctggaaaaggacatttatcctagagggcacagggggtgtctctctggtaggggaagggtggggaggtggctttataagagtggtctgccttctccctttctcacttttcctcaccccttttctctcttcccccgcaaagctgcttccctgccctgccaccacctttagtgctttgtcttttttcccctttgcccatgctcagctgttaacccataaagacttcgttgattttgtgtgcatagtggatggtatggctgcattaatcccttcactgcctgtataccctagaatttgtccctgacactgacttcagagcatggtttgagttcatctcccatcattccccattgttgtgcttcccgtaaaaactgccagctttatcatttcccctggctctgcccacactgcatgtgtaggggctgaactatgggcaagtgtctgaccacccaggcaggtgagtgtgtgtcttctaatgcaagtctgtttctgtttttgttgtctttttaaactcatagaattgattgttgaaaataaggccatcaactgctaaaacaactactaaaataattctttttaatataaaaataactttgtcaaattcactttcagaagatttttcagatgtccctgttgagagcattgttctagataggttatatttgaaactgtgagcagaagcatgtgagcccatctgctatgatgagtaatagtcattgaggcctgaaacatacagtgctttaagcatgactgttattacaaagcatgcttctcccaccccacccaccccctcaaagaaggtagccattgaaacataaggatgatagatagaatgtattacttcaaatctaactcttagctggtggaggatttagtaatttagttgctttaggtcttgtaaaagctcctgccgctaactttaggagatgagaagtttgacccttaatgttcttgatatttttttagatcaactccacaatttactgtgatccaatccatctgctttctatctgttgtgctctatgattggttctcatttaccttcatttctgtattctactttccttaaactttaaggaaatctaatcacaactcctgaagacttacctttcttagatctgaaacttaagatcagtgtattataaaatggaatctcttagcagtcacagctacataaattgggattttaatagttgtctgtgctttgaattcttttcctttaaatgtctgtttcttttatgtaaagtttttcagtttggggaacgtgtagtcttcccctcccttttaatttctcaccaggatctaaaccccccttctctgtgaagcttaaatctgcattgtactctccctcctccccccccatcagtatccagcaggttacccttcagataaagaagggaagaagcctaaaggacagtcaaagaagcagcccagtggaaccacaaaaaggccaatttcagatgatgactgtccaagtgcctccaaagtgtacaaagcatcagattcagcagaagcaattgaggcttttcaactaactcctcaacagcaacatctcatcagagaagattgtcaaaaccagaagctgtgggatgaagtgctttcacatcttgtggaaggaccaaattttctgaaaaaattggaacaatcttttatgtgcgtttgctgtcaggagctagtttaccagcctgtgacaactgagtgcttccacaatgtctgtaaagattgcctacagcgctcctttaaggcacaggttttctcctgccctgcttgccggcatgatcttggccagaattacatcatgattcccaatgagattctgcagactctacttgaccttttcttccctggctacagcaaaggacgatgatctgcctgctttcactgtgttgttcatggtggctttttggacaataaagaatctaaaatgggtggggagggtggaagaaatggtggactgtatctctcacgttctgaagcagctaatcctctttcccacatagccatcatcttgtgtgtgtagtaagaggcccatttctcaactgtcttttaaatatctaaaggtagttcctgtaacaactagttttaatgagtaaaaagtcaaagcctcagctctagttgatatccaagttatgatttattttgcaactacctcaggacagaaaagatttatggggattttaaaaatcattgaataactagttaaatgaaattttagctacacactgcctcccaaatattagttgtgcctggttcttgtaatttgattttacagaaaaggaaatgacacttgagatccttggaatgaacacagcttctaaagtgtgcatatacttttttaacgtctcttcttccattacaatgtgtgttttgcaaggacaggttcattttttttagcccactttgtgaactccattgtgcttttttctggtgttttatgcaagttgactactaatgactaatgagaacaataatgaatgcattgttgctgcattagtgtaatgtggtgtggttttgcacttaaaataggtattcatatgctctagttgtaaatgttcatgaaaatccacttctctactagtcgaactgcttttagtgtctcaccagtggttttacatctgcagagttttgagggctgtgctgacctttgagaggatttgaaattgcttcatattgtgatcctaaattttatattcactatattccctaaagtataccttaataaatattttatgatcagaaaaacagct

SEQ ID NO: 283 - DNA, 1072 bp, Homo sapiens; identical to SEQ ID NO: 105

SEQ ID NO: 284 - DNA, 1775 bp, Homo sapiens
atggacggcaacgacaacgtgaccctgctcttcgcccctctgctgcgggacaactacaccctggcgcccaatgccagcagcctgggccccggcacggacctcgccctcgcccctgcctccagcgccggccccggccctgggctcagcctcgggccgggtccgagcttcggcttcagccccggccccactccgaccccggagcccacgaccagcggcctcgcgggcggcgcggcgagccacggcccttccccgttccctcggccctgggcgccccacgcgctcccgttctgggacacgccgctgaaccacgggctgaacgtgttcgtgggcgccgccctgtgcatcaccatgctgggcctgggctgcacggtggacgtgaaccacttcggggcgcacgtccgtcggcccgtgggcgcgctgctggcagcgctctgccagttcggcctcctgccgctgctggccttcctgctggccctcgccttcaagctggacgaggtggccgccgtggcggtgctcctgtgtggctgctgtcccggcggcaatctctccaatcttatgtccctgctggttgacggcgacatgaacctcagacgtgctgctctcttggcactctcctcggatgtaggttctgcccagacttcaaccccgggacttgcagtctccccgttccacctctactcaacatacaagaaaaaggttagctggctgtttgactcaaagctcgttctgatttctgcacattcccttttctgcagcatcatcatgaccatctcctccacgcttctggccctcgtcttgatgcccctgtgcctgtggatctacagctgggcttggatcaacacccctatcgtgcagttactacccctagggaccgtgaccctgactctctgcagcactctcatacctatcgggttgggcgtcttcattcgctacaaatacagccgggtggctgactacattgtgaaggtttccctgtggtctctgctagtgactctggtggtccttttcataatgaccggcactatgttaggacctgaactgctggcaagtatccctgcagctgtttatgtgatagcaatttttatgcctttggcaggctacgcttcaggttatggtttagctactctcttccatcttccacccaactgcaagaggactgtatgtctggaaacaggtagtcagaatgtgcagctctgtacagccattctaaaactggcctttccaccgcaattcataggaagcatgtacatgtttcctttgctgtatgcacttttccagtctgcagaagcggggatttttgttttaatctataaaatgtatggaagtgaaatgttgcacaagcgagatcctctagatgaagatgaagatacagatatttcttataaaaaactaaaagaagaggaaatggcagacacttcctatggcacagtgaaagcagaaaatataataatgatggaaaccgctcagacttctctctaaatgtggagatacacaggagcttctatcttgctgaaatattgcttcatatttatagcctgtggtagtgcacatggttaacataaaagataacactggttcacatcatacatgtaacaattctgatctttttaaggttcactggtgtattaaccaaacgttgtcacaaattacaaatcaatgctgtaatataatttgcacctggaatggctaacgtgaagcctgaattaaatgtggtttttagtttttaccatcaccaatttctatgactgttgcaaatacagaatctattagaaaac

SEQ ID NO: 285 - DNA, 22 bp, Mus musculus
cctatgggtctgtgaccaacgt

SEQ ID NO: 286 - DNA, 22 bp, Mus musculus
ccatcttctacccggaacttgt

SEQ ID NO: 287 - DNA, 20 bp, Mus musculus
catggccttccgtgttccta

SEQ ID NO: 288 - DNA, 21 bp, Mus musculus
cctgcttcaccaccttcttga

SEQ ID NO: 289 - protein, 15 aa, Homo sapiens
CVPISDSKSIQKSEL

SEQ ID NO: 290 - protein, 13 aa, Homo sapiens
QNGNITAKGPSIQ

SEQ ID NO: 291 - protein, 13 aa, Homo sapiens
DASCMSQRRPKCR

SEQ ID NO: 292 - protein, 13 aa, Homo sapiens
EIIKLTMKFQALR

SEQ ID NO: 293 - protein, 14 aa, Homo sapiens
VHKAESSTDSSGPL

SEQ ID NO: 294 - protein, 14 aa, Homo sapiens
PQLMRTKSDASCMS

SEQ ID NO: 295 - protein, 14 aa, Homo sapiens
MPVLDSFVEKLKEE

SEQ ID NO: 296 - protein, 14 aa, Homo sapiens
TSWMPRRPSCPLKE

SEQ ID NO: 297 - protein, 14 aa, Homo sapiens
SPQNGNITAKGPSI

SEQ ID NO: 298 - protein, 14 aa, Homo sapiens
TNVRVNSTMTTLQV

SEQ ID NO: 299 - protein, 16 aa, Homo sapiens
KSELLGLLKTYNCYHE

SEQ ID NO: 300 - protein, 16 aa, Homo sapiens
PSPQNGNITAKGPSIQ

SEQ ID NO: 301 - protein, 16 aa, Homo sapiens
FTPAYGSVTNVRVNST

SEQ ID NO: 302 - protein, 17 aa, Homo sapiens
SGERTKLKDCEYPLISR

SEQ ID NO: 303 - DNA, 29 bp, Homo sapiens
gaattcttgtctgcagacaagaggaagag

SEQ ID NO: 304 - DNA, 29 bp, Homo sapiens
gcggccgcttacttggcctccaccagctg

SEQ ID NO: 305 - DNA, 23 bp, Homo sapiens
gcttgaccgacagttgcatgaag

SEQ ID NO: 306 - DNA, 32 bp, Artificial Sequence
  AD037 Mutagenic Primer For Creating Myristylation Site Mutant.
gaagtcggagctcttaaactgctaccatgagg

SEQ ID NO: 307 - DNA, 30 bp, Artificial Sequence
  AD037 Mutagenic Primer For Creating Ras Motif Mutant.
ttctctatcaacggcgtggaagtcccccat

(19) United States
(12) Patent Application Publication
(10) Pub. No.: US 2004/0086896 A1
(43) Pub. Date: May 6, 2004

(54) Polynucleotides and polypeptides associated with the NF-kB pathway

(76) Inventors: Julie Carman, Lawenceville, NJ (US); John N. Feder, Belle Mead, NJ (US); Steven G. Nadler, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/431,096

(22) Filed: May 7, 2003

Related U.S. Application Data
(63) Continuation-in-part of application No. 10/126,103, filed on Apr. 19, 2002.
(60) Provisional application No. 60/284,962, filed on Apr. 19, 2001.
(60) Provisional application No. 60/286,645, filed on Apr. 26, 2001.
(60) Provisional application No. 60/346,986, filed on Jan. 9, 2002.

(51) Int. Cl. C12Q 1/68; C07H 21/04; C12P 21/02; C07K 14/52; C07K 16/24

(52) U.S. Cl. 435/6; 435/69.5; 435/320.1; 435/325; 530/351; 530/388.23; 536/23.5

(57) ABSTRACT

The present invention provides polynucleotides encoding NF-kB-associated polypeptides, fragments and homologues thereof. Also provided are vectors, host cells, antibodies, and recombinant and synthetic methods for producing said polypeptides. The invention further relates to diagnostic and therapeutic methods for applying these NF-kB-associated polypeptides to the diagnosis, treatment, and/or prevention of various diseases and/or disorders related to these polypeptides. The invention further relates to screening methods for identifying agonists and antagonists of the polynucleotides and polypeptides of the present invention.

This application is a continuation-in-part application of non-provisional application U.S. Ser. No. 10/126,103, filed Apr. 19, 2002, which claims benefit to provisional application U.S. Serial No. 60/284,962 filed Apr. 19, 2001; to provisional application U.S. Serial No. 60/286,645, filed Apr. 26, 2001; and to provisional application U.S. Serial No. 60/346,986, filed Jan. 9, 2002. The entire teachings of the referenced applications are incorporated herein by reference.

### FIELD OF THE INVENTION

The present invention provides polynucleotides encoding NF-kB-associated polypeptides, fragments and homologues thereof. Also provided are vectors, host cells, antibodies, and recombinant and synthetic methods for producing said polypeptides. The invention further relates to diagnostic and therapeutic methods for applying these NF-kB-associated polypeptides to the diagnosis, treatment, and/or prevention of various diseases and/or disorders related to these polypeptides. The invention further relates to screening methods for identifying agonists and antagonists of the polynucleotides and polypeptides of the present invention.

### BACKGROUND OF THE INVENTION

Members of the NF-kB family of transcription factors are critical regulators of inflammatory and stress responses. In humans, the family consists of five members (NF-kB1 p50/p105; NF-kB2 p52/p100; c-Rel, RelA p65; and RelB) that share a conserved 300 amino acid Rel Homology Domain (RHD). The RHD is required for dimerization, DNA binding, and association with members of the IkB family. Members of the NF-kB family hetero and homodimerize to form active complexes. The complexes differ in their ability to activate transcription, with p65 and c-Rel containing the most potent activation domains. Complexes of p50 and p52 homodimers are thought to act as transcriptional repressors since these proteins lack activation domains. The most abundant complex in the majority of cells consists of p50/p65 heterodimers.

In resting cells, NF-kB complexes reside in the cytosol in association with inhibitory proteins, IkB, that mask the NF-kB nuclear localization sequence thereby preventing translocation. The IkB family consists of five family members—IkBα, IkBβ, IkB□, IkBγ, and Bcl-3. Each family member contains 6-7 ankyrin repeat domains that form a curved alpha helical stack which interacts with the Ig-like folds of the RHD (Jacobs et al. (1998) Cell 95:749-758). The precursors of p50 (p105) and p52 (p100) also contain multiple ankyrin repeats in the C terminal half of the molecule. These precursor proteins can associate with other Rel family members, thereby retaining them in an inactive state in the cytosol. Generation of mature p50 and p52 subunits is thought to involve limited proteolysis of the precursor proteins by the proteasome (Fan et al. (1991) Nature 354:395-398). Cotranslational processing has also been reported (Lin et al. (1998) Cell 92:819-828).

A wide variety of stimuli activate NF-kB including TNFα, IL-1, growth factors, T cell activation signals, LPS, dsRNA, phorbol esters, okadaic acid, HIV-Tax, UV light, and ionizing radiation. In response to these stimuli, IkB is rapidly phosphorylated on two serine residues (Ser 32, Ser 36). A large molecular weight complex consisting of two serine/threonine protein kinases, IKK-1 and IKK-2 (Zandi et al. (1997) Cell 91:243-252), and a non-catalytic regulatory subunit IKK-γ (Rothwarf et al. (1998) Nature 395:297-300), has been shown to phosphorylate both serine residues of IkB. It is not yet clear how the activity of this complex is regulated by upstream activators. Germline knockouts of each of the components of this complex has suggested that the kinases may play distinct roles in NF-kB activation pathways. Mice deficient in IKK-1 die perinatally and exhibit defects in limb and tail development, and in epidermal differentiation (Hu et al. (1999) Science 284:316-320). Activation of NF-kB in response to pro-inflammatory stimuli was normal in these animals. In contrast, IKK-2 deficient animals showed no activation of NF-kB in response to IL-1, LPS, or TNFα stimulation (Li et al. (1999) Science 284:321-325). Limb, tail development, and epidermal differentiation were all normal. These animals died before birth due to massive liver apoptosis, a phenotype very similar to the RelA (p65) deficient animals (Doi et al. (1997) J. Exp. Med. 185:953-961).

Although it lacks catalytic activity, IKK-γ is a critical component of the IKK complex. Mice deficient for IKK-γ failed to activate either the IKK complex or NF-kB in response to a variety of stimuli including TNFα, IL-1, LPS, and poly (IC) (Rudolph et al. (2000) Genes Dev. 14:854-862). These animals died in utero at an earlier stage than either the IKK-1 or IKK-2 knockouts due to massive liver apoptosis.

Following phosphorylation by the IKK complex, IkB is a recognized by a SCF E3 ubiquitin ligase that recruits an E2 enzyme. The E2/E3 complex attaches a polyubiquitin chain to IkB (Yaron et al. (1998) Nature 396:590-594). Ubiquitinated IkB is rapidly degraded by the 26S proteasome, thereby unmasking the NF-kB nuclear localization sequence and allowing translocation of the complex into the nucleus.

Once in the nucleus, NF-kB activates the transcription of a number of target genes including cytokines, cytokine receptors, chemokines, adhesion molecules, acute phase proteins, anti-apoptotic proteins, and enzymes including iNOS and COX-2 (Pahl (1999) Oncogene 18:6853-6866). Many of these target genes are pro-inflammatory and have been linked to disease pathology.

Aberrant NF-kB activity is associated with a number of human diseases. Mutations or truncations of IkB have been observed in some Hodgkins lymphomas (Cabannes et al. (1999) Oncogene 18:3063-3070). Genes encoding p65, p105, and p100 have been reported to be overexpressed or rearranged in some solid and hematopoietic tumors (Rayet et al. (1999) Oncogene 18:6938-6947). Missense mutations in IKKγ have been seen in some hyper-IgM syndromes characterized by hypohydrotic ectodermal dysplasia (Jain et al. (2001) Nature Immunol.2:223-228), and in cases of X-linked anhidrotic ectodermal dysplasia with immunodeficiency (Doffinger et al. (2001) Nature Genet. 27:277-285). Genome rearrangements in IKKγ have also been observed in cases of familial incontinentia pigmenti (The International Incontinentia Pigmenti Consortium (2000) Nature 405:466-472).

In addition to the above genetic diseases, NF-kB is involved in many viral infections (Hiscott et al. (2001) J. Clin. Invest. 107:143-151). Several families of viruses including HIV-1, HTLV-1, hepatitis B, hepatitis C, EBV, and influenza activate NF-kB. The mechanisms of activation are distinct, and in some cases have not been well characterized. Some viral proteins have been identified that activate NF-kB including influenza virus hemagglutinin, matrix protein, and nucleoprotein; hepatitis B nucleoprotein and HBx protein; hepatitis C core protein; HTLV-1 Tax protein; HIV-1 Tat protein; and EBV LMP1 protein. The activation of NF-kB in target cells facilitates viral replication, host cell survival, and evasion of immune responses.

Many inflammatory diseases are associated with constitutive nuclear NF-kB localization and transcriptional activity. NF-kB is activated in the inflamed synovium of rheumatoid arthritis patients (Marok et al. (1996) Arthritis Rheum. 39:583-591) and in animal models of arthritis (Miagkov et al. (1998) Proc. Natl. Acad. Sci. USA 95:13859-13864). Gene transfer of a dominant negative IkBα significantly inhibited TNFα secretion by human synoviocytes (Bondeson et al. (1999) Proc. Natl. Acad. Sci. USA 96:5668-5673). In animal models of inflammatory bowel disease, treatment with antisense p65 oligonucleotides significantly inhibited clinical and histological signs of colitis (Neurath et al. Nature Med. 2:998-1004). NF-kB has also been associated with other inflammatory diseases including asthma, atherosclerosis, cachexia, euthyroid sick syndrome, and stroke (Yamamoto et al. (2001) J. Clin. Invest. 107:135-142).

Consistent with the involvement of NF-kB in inflammatory diseases, a number of anti-inflammatory therapies inhibit NF-kB activation. Glucocorticoids inhibit NF-kB by a variety of mechanisms including upregulation of IkBα transcription (Scheinman et al. (1995) Science 270:283-286), direct interference with NF-kB dependent transactivation (DeBosscher et al. (1997) Proc. Natl. Acad. Sci. USA 94:13504-13509), competition for transcriptional coactivators (Sheppard et al. (1998) J. Biol. Chem. 273:29291-29294), association with the catalytic subunit of protein kinase A (Doucas et al. (2000) Proc. Natl. Acad. Sci. USA 97:11893-11898), and by interfering with serine-2 phosphorylation of the RNA polymerase II carboxy-terminal domain (Nissen et al. (2000) Genes Dev. 14:2314-2329). Several NSAIDs including aspirin (Yin et al. (1998) Nature 396:77-80), sulindac (Yamamoto et al. (1999) J. Biol. Chem. 274:27307-27314), and cyclopentenone prostaglandins (Rossi et al. (2000) Nature 403:103-118) inhibit IKK activation. The potent anti-inflammatories, sesquiterpene lactones (Hehner et al. (1998) J. Biol. Chem. 273:1288-1297) and sulfasalazine (Wahl et al. (1998) J. Clin. Invest. 101:1163-1174), block IkBα and IkBβ degradation. Gold compounds which have been used to treat rheumatoid arthritis were shown to inhibit both IKK activation (Jeon et al. (2000) J. Immunol. 164:5981-5989), and NF-kB DNA binding (Yang et al. (1995) FEBS Letters 361:89-96). The anti-inflammatory compound deoxyspergualin was shown to block NF-kB nuclear translocation (Tepper et al. (1995) J. Immunol. 155:2427-2436). Proteasome inhibitors have recently been shown to inhibit inflammation and disease progression in animal models of arthritis, asthma, and EAE (Palombella et al. (1998) Proc. Natl. Acad. Sci. USA 95:15671-15676).

The association of NF-kB with a number of human diseases suggests that components of this pathway will have utility as therapeutic targets for the treatment of these diseases. As described herein, the novel NF-kB target genes were identified by utilizing a selective NF-kB inhibitor. The inhibitor consists of a permeable D-amino acid peptide carrying two nuclear localization sequences derived from the SV40 large T antigen (as described in U.S. Pat. No. 5,877,282). This peptide selectively blocked NF-kB nuclear localization in a dose-dependent manner resulting in inhibition of kappa Ig expression and surface CD40 in B cells, TNFα and IL-6 production in macrophages, and T cell proliferation (Fujihara et al. (2000) J. Immunol. 165:1004-1012). In vivo, the peptide suppressed humoral responses and was efficacious in a septic shock model and a model of inflammatory bowel disease. A human monocyte line was stimulated with the NF-kB activator lipopolysaccharide (LPS) in the presence and absence of compound peptide A (See FIG. 1), or dexamethasone. Genes that were differentially expressed in these groups were identified by the generation of a subtraction library, and by probing microarrays.

Using the above examples, it is clear the availability of novel cloned NFkB associated polynucleotides and polypeptides provides an opportunity for adjunct or replacement therapy, and may be useful for the identification of NFkB agonists, or stimulators (which might stimulate and/or bias NFkB action), as well as, in the identification of NFkB inhibitors. All of which might be therapeutically useful under different circumstances.

The present invention also relates to recombinant vectors, which include the isolated nucleic acid molecules of the present invention, and to host cells containing the recombinant vectors, as well as to methods of making such vectors and host cells, in addition to their use in the production of NFkB associated polypeptides or peptides using recombinant techniques. Synthetic methods for producing the polypeptides and polynucleotides of the present invention are provided. Also provided are diagnostic methods for detecting diseases, disorders, and/or conditions related to the NFkB associated polypeptides and polynucleotides, and therapeutic methods for treating such diseases, disorders, and/or conditions. The invention further relates to screening methods for identifying binding partners of the polypeptides.

### BRIEF SUMMARY OF THE INVENTION

The present invention provides isolated nucleic acid molecules, that comprise, or alternatively consist of, a polynucleotide sequence referenced in Tables I, II, III, or IV, in addition to polynucleotide sequences encoding NFkB associated polypeptides having the amino acid sequences referenced in Tables I, II, III, or IV.

The present invention also relates to recombinant vectors, which include the isolated nucleic acid molecules of the present invention, and to host cells containing the recombinant vectors, as well as to methods of making such vectors and host cells, in addition to their use in the production of NFkB associated polypeptides or peptides using recombinant techniques. Synthetic methods for producing the polypeptides and polynucleotides of the present invention are provided. Also provided are diagnostic methods for detecting diseases, disorders, and/or conditions related to the NFkB associated polypeptides and polynucleotides, and therapeutic methods for treating such diseases, disorders, and/or conditions. The invention further relates to screening methods for identifying binding partners of the polypeptides.

The invention further provides an isolated NFkB associated polypeptide having an amino acid sequence encoded by a polynucleotide described herein.

The invention further relates to a polynucleotide encoding a polypeptide fragment of a member of the group consisting of SEQ ID NO:109-118, 126, 128, 144-152, 160, and 161.

The invention further relates to a polynucleotide encoding a polypeptide domain of a member of the group consisting of SEQ ID NO:109-118, 126, 128, 144-152, 160, and 161.

The invention further relates to a polynucleotide encoding a polypeptide epitope of a member of the group consisting of SEQ ID NO:109-118, 126, 128, 144-152, 160, and 161.

The invention further relates to a polynucleotide encoding a polypeptide of a member of the group consisting of SEQ ID NO:109-118, 126, 128, 144-152, 160, and 161 having NFkB modulating activity.

The invention further relates to a polynucleotide encoding a polypeptide of a member of the group consisting of SEQ ID NO:109-118, 126, 128, 144-152, 160, and 161 which is modulated by NFkB or the NFkB pathway.

The invention further relates to a polynucleotide which represents the complimentary sequence (antisense) of a member of the group consisting of SEQ ID NO:1-108, 125, 127, 132-140, 158-159, and 264-284.

The invention further relates to a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues.

The invention further relates to an isolated nucleic acid molecule of a member of the group consisting of SEQ ID NO:109-118, 126, 128, 144-152, 160, and 161, wherein the polynucleotide fragment comprises a nucleotide sequence encoding a NFkB associated protein.

The invention further relates to an isolated nucleic acid molecule of a member of the group consisting of SEQ ID NO:1-108, 125, 127, 132-140, 158-159, and 264-284, wherein the polynucleotide fragment comprises a nucleotide sequence encoding the sequence identified as a member of the group consisting of SEQ ID NO:109-118, 126, 128, 144-152, 160, and 161 which is hybridizable to SEQ ID NO:1-108, 125, 127, 132-140, 158-159, and 264-284.

The invention further relates to an isolated nucleic acid molecule of of a member of the group consisting of SEQ ID NO:1-108, 125, 127, 132-140, 158-159, and 264-284, wherein the polynucleotide fragment comprises the entire nucleotide sequence of a member of the group consisting of SEQ ID NO:1-108, 125, 127, 132-140, 158-159, and 264-284.

The invention further relates to an isolated nucleic acid molecule of a member of the group consisting of SEQ ID NO:1-108, 125, 127, 132-140, 158-159, and 264-284, wherein the nucleotide sequence comprises sequential nucleotide deletions from either the C-terminus or the N-terminus.

The invention further relates to an isolated polypeptide comprising an amino acid sequence that comprises a polypeptide fragment of a member of the group consisting of a member of the group consisting of SEQ ID NO:109-118, 126, 128, 144-152, 160, and 161.

The invention further relates to a polypeptide fragment of a member of the group consisting of a member of the group consisting of SEQ ID NO:109-118, 126, 128, 144-152, 160, and 161 having NFkB modulating activity.

The invention further relates to a polypeptide fragment of a member of the group consisting of a member of the group consisting of SEQ ID NO:109-118, 126, 128, 144-152, 160, and 161 which is modulated by NFkB or the NFkB pathway.

The invention further relates to a polypeptide domain of a member of the group consisting of a member of the group consisting of SEQ ID NO:109-118, 126, 128, 144-152, 160, and 161.

The invention further relates to a polypeptide epitope of a member of the group consisting of a member of the group consisting of SEQ ID NO:109-118, 126, 128, 144-152, 160, and 161.

The invention further relates to a full length protein of a member of the group consisting of a member of the group consisting of SEQ ID NO:109-118, 126, 128, 144-152, 160, and 161.

The invention further relates to a variant of a member of the group consisting of a member of the group consisting of SEQ ID NO:109-118, 126, 128, 144-152, 160, and 161.

The invention further relates to an allelic variant of a member of the group consisting of a member of the group consisting of SEQ ID NO:109-118, 126, 128, 144-152, 160, and 161.

The invention further relates to a species homologue of a member of the group consisting of a member of the group consisting of SEQ ID NO:109-118, 126, 128, 144-152, 160, and 161.

The invention further relates to the isolated polypeptide of of a member of the group consisting of SEQ ID NO:109-118, 126, 128, 144-152, 160, and 161, wherein the full length protein comprises sequential amino acid deletions from either the C-terminus or the N-terminus.

The invention further relates to an isolated antibody that binds specifically to the isolated polypeptide of a member of the group consisting of SEQ ID NO:109-118, 126, 128, 144-152, 160, and 161.

The invention further relates to a method for preventing, treating, or ameliorating a medical condition, comprising administering to a mammalian subject a therapeutically effective amount of the polypeptide of a member of the group consisting of SEQ ID NO:109-118, 126, 128, 144-152, 160, and 161 or the polynucleotide of a member of the group consisting of SEQ ID NO:1-108, 125, 127, 132-140, 158-159, and 264-284.

The invention further relates to a method of diagnosing a pathological condition or a susceptibility to a pathological condition in a subject comprising the steps of (a) determining the presence or absence of a mutation in the polynucleotide of a member of the group consisting of SEQ ID NO:1-108, 125, 127, 132-140, 158-159, and 264-284; and (b) diagnosing a pathological condition or a susceptibility to a pathological condition based on the presence or absence of said mutation.

The invention further relates to a method of diagnosing a pathological condition or a susceptibility to a pathological condition in a subject comprising the steps of (a) determining the presence or amount of expression of the polypeptide of a member of the group consisting of SEQ ID NO:109-118, 126, 128, 144-152, 160, and 161 in a biological sample; and diagnosing a pathological condition or a susceptibility to a pathological condition based on the presence or amount of expression of the polypeptide.

The invention further relates to a method for identifying a binding partner to the polypeptide of a member of the group consisting of SEQ ID NO:109-118, 126, 128, 144-152, 160, and 161 comprising the steps of (a) contacting the polypeptide of a member of the group consisting of SEQ ID NO:109-118, 126, 128, 144-152, 160, and 161 with a binding partner; and (b) determining whether the binding partner effects an activity of the polypeptide.

The invention further relates to a gene corresponding to the cDNA sequence of a member of the group consisting of SEQ ID NO:1-108, 125, 127, 132-140, 158-159, and 264-284.

The invention further relates to a method of identifying an activity in a biological assay, wherein the method comprises the steps of (a) expressing SEQ ID NO:1-108, 125, 127, 132-140, 158-159, and 264-284 in a cell, (b) isolating the supernatant; (c) detecting an activity in a biological assay; and (d) identifying the protein in the supernatant having the activity.

The invention further relates to a process for making polynucleotide sequences encoding gene products having altered activity selected from the group consisting of a member of the group consisting of SEQ ID NO:109-118, 126, 128, 144-152, 160, and 161 activity comprising the steps of (a) shuffling a nucleotide sequence of a member of the group consisting of SEQ ID NO:1-108, 125, 127, 132-140, 158-159, and 264-284, (b) expressing the resulting shuffled nucleotide sequences and, (c) selecting for altered activity selected from the group consisting of a member of the group consisting of SEQ ID NO:109-118, 126, 128, 144-152, 160, and 161 activity as compared to the activity selected from the group consisting of a member of the group consisting of SEQ ID NO:109-118, 126, 128, 144-152, 160, and 161 activity of the gene product of said unmodified nucleotide sequence.

The invention further relates to a shuffled polynucleotide sequence produced by a shuffling process, wherein said shuffled DNA molecule encodes a gene product having enhanced tolerance to an inhibitor of any one of the activities selected from the group consisting of a member of the group consisting of SEQ ID NO:109-118, 126, 128, 144-152, 160, and 161 activity.

The invention further relates to a method for diagnosing, preventing, treating, or ameliorating a medical condition with the polypeptide provided as a member of the group consisting of SEQ ID NO:109-118, 126, 128, 144-152, 160, and 161, in addition to, its encoding nucleic acid, wherein the medical condition is an inflammatory disorder

The invention further relates to a method for diagnosing, preventing, treating, or ameliorating a medical condition with the polypeptide provided as a member of the group consisting of SEQ ID NO:109-118, 126, 128, 144-152, 160, and 161, in addition to, its encoding nucleic acid, wherein the medical condition is a disorder associated with NFkB signaling.

The invention further relates to a method for diagnosing a medical condition associated with aberrant NFkB activity using probes or primer pairs specific to a member of the group consisting of: (i) a polynucleotide encoding a polypeptide fragment of a member of the group consisting of SEQ ID NO:109-118, 126, 128, 144-152, 160, and 161; (ii) a polynucleotide encoding a polypeptide domain of a member of the group consisting of SEQ ID NO:109-118, 126, 128, 144-152, 160, and 161; (iii) a polynucleotide encoding a polypeptide epitope of a member of the group consisting of SEQ ID NO:109-118, 126, 128, 144-152, 160, and 161; (iv) a polynucleotide encoding a polypeptide of a member of the group consisting of SEQ ID NO:109-118, 126, 128, 144-152, 160, and 161 having NFkB modulating activity; (v) a polynucleotide encoding a polypeptide of a member of the group consisting of SEQ ID NO:109-118, 126, 128, 144-152, 160, and 161 which is modulated by NFkB or the NFkB pathway; (vi) a polynucleotide which represents the complimentary sequence (antisense) of a member of the group consisting of SEQ ID NO:1-108, 125, 127, 132-140, 158-159, and 264-284; (vii) a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues; (viii) an isolated nucleic acid molecule of a member of the group consisting of SEQ ID NO:109-118, 126, 128, 144-152, 160, and 161, wherein the polynucleotide fragment comprises a nucleotide sequence encoding a NFkB associated protein; (ix) an isolated nucleic acid molecule of a member of the group consisting of SEQ ID NO:1-108, 125, 127, 132-140, 158-159, and 264-284, wherein the polynucleotide fragment comprises a nucleotide sequence encoding the sequence identified as a member of the group consisting of SEQ ID NO:109-118, 126, 128, 144-152, 160, and 161,which is hybridizable to SEQ ID NO:1-108, 125, 127, 132-140, 158-159, and 264-284; and (x) an isolated nucleic acid molecule of of a member of the group consisting of SEQ ID NO:1-108, 125, 127, 132-140, 158-159, and 264-284, wherein the polynucleotide fragment comprises the entire nucleotide sequence of a member of the group consisting of SEQ ID NO:1-108, 125, 127, 132-140, 158-159, and 264-284; wherein said method comprises the step of using said probe or primer pair to correlate expression of said member to a disease or disorder associated with said member.

The invention further relates to a method of identifying a compound that modulates the biological activity of an NFkB associated polypeptide, comprising the steps of, (a) combining a candidate modulator compound with an NFkB associated polypeptide having the sequence set forth in a member of the group consisting of SEQ ID NO:109-118, 126, 128, 144-152, 160, and 161; and measuring an effect of the candidate modulator compound on the activity of an NFkB associated polypeptide.

The invention further relates to a method of identifying a compound that modulates the biological activity of an NFkB associated polypeptide, comprising the steps of, (a) combining a candidate modulator compound with a host cell expressing an NFkB associated polypeptide having the sequence as set forth in SEQ ID NO:109-118, 126, 128, 144-152, 160, and 161; and (b) measuring an effect of the candidate modulator compound on the activity of the expressed an NFkB associated polypeptide.

The invention further relates to a method of identifying a compound that modulates the biological activity of an NFkB associated polypeptide, comprising the steps of, (a) combining a candidate modulator compound with a host cell containing a vector described herein, wherein an NFkB associated polypeptide is expressed by the cell; and, (b) measuring an effect of the candidate modulator compound on the activity of the expressed an NFkB associated polypeptide.

The invention further relates to a method of screening for a compound that is capable of modulating the biological activity of an NFkB associated polypeptide, comprising the steps of: (a) providing a host cell described herein; (b) determining the biological activity of an NFkB associated polypeptide in the absence of a modulator compound; (c) contacting the cell with the modulator compound; and (d) determining the biological activity of an NFkB associated polypeptide in the presence of the modulator compound; wherein a difference between the activity of an NFkB associated polypeptide in the presence of the modulator compound and in the absence of the modulator compound indicates a modulating effect of the compound.

The invention further relates to a method of screening for a compound that is capable of modulating the biological activity of NFkB associated polypeptide comprising a member of the group consisting of (i) an amino acid sequence that comprises a polypeptide fragment of a member of the group consisting of SEQ ID NO:109-118, 126, 128, 144-152, 160, and 161; (ii) a polypeptide fragment of a member of the group consisting of SEQ ID NO:109-118, 126, 128, 144-152, 160, and 161 having NFkB modulating activity; (iii) a polypeptide fragment of a member of the group consisting of SEQ ID NO:109-118, 126, 128, 144-152, 160, and 161 which is modulated by NFkB or the NFkB pathway; (iv) a polypeptide domain of a member of the group consisting of SEQ ID NO:109-118, 126, 128, 144-152, 160, and 161; (v) a polypeptide epitope of a member of the group consisting of SEQ ID NO:109-118, 126, 128, 144-152, 160, and 161; (vi) a full length protein of a member of the group consisting of SEQ ID NO:109-118, 126, 128, 144-152, 160, and 161; (vii) a variant of a member of the group consisting of SEQ ID NO:109-118, 126, 128, 144-152, 160, and 161; (viii) an allelic variant of a member of the group consisting of SEQ ID NO:109-118, 126, 128, 144-152, 160, and 161; and (ix) a species homologue of a member of the group consisting of SEQ ID NO:109-118, 126, 128, 144-152, 160, and 161; wherein the method comprises the steps of: (a) providing a host cell described herein; (b) determining the biological activity of an NFkB associated polypeptide or a member of the group above in the absence of a modulator compound; (c) contacting the cell with the modulator compound; and (d) determining the biological activity of an NFkB associated polypeptide or a member of the group above in the presence of the modulator compound; wherein a difference between the activity of an NFkB associated polypeptide or a member of the group above in the presence of the modulator compound and in the absence of the modulator compound indicates a modulating effect of the compound.

The invention further relates to a compound that modulates the biological activity of a NFkB associated polypeptide as identified by the methods described herein.

The invention further relates to a compound that modulates the biological activity of NFkB, or affects the NFkB pathway, either directly or indirectly as identified by the methods described herein.

The invention further relates to method for diagnosing a polymorphism associated with predisposition to an NFkB associated disorder selected from the group consisting of immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE, in a human comprising: detecting a germline alteration of a wild-type NFkB associated gene or its expression products in a human sample wherein said NFkB associated gene or said expression product is a nucleic acid or a polypeptide defined by any one of the group of SEQ ID NO:1-108, 125, 127, 132-140, 158-159, and 264-284, said alteration indicating a predisposition to at least one of said NFkB associated disorders.

The invention further relates to a method for diagnosing, preventing, treating, or ameliorating a medical condition with an antibody directed against a polypeptide provided as a member of the group consisting of SEQ ID NO:109-118, 126, 128, 144-152, 160, and 161, wherein the disorder is a NFkB associated disorder selected from the group consisting of immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE, or additional disorders described herein in a human.

The invention further relates to a method for diagnosing, preventing, treating, or ameliorating a medical condition with an antibody directed against a polypeptide encoded by a polynucleotide that is a member selected from the group consisting of SEQ ID NO:1-108, 125, 127, 132-140, 158-159, and 264-284, wherein the disorder is an NFkB associated disorder selected from the group consisting of immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE, or additional disorders described herein in a human.

The invention further relates to a method for diagnosing, preventing, treating, or ameliorating a medical condition with an antisense oligonucleotide directed against a polypeptide encoded by a polynucleotide that is a member selected from the group consisting of SEQ ID NO:1-108, 125, 127, 132-140, 158-159, and 264-284, wherein the disorder is an NFkB associated disorder selected from the group consisting of immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE, or additional disorders described herein in a human.

### BRIEF DESCRIPTION OF THE FIGURES/DRAWINGS

FIG. 1 provides the amino acid sequence of the NFkB inhibitory peptide (SEQ ID NO:124) that was used in identifying the NFkB-associated polynucleotides and polypeptides of the present invention. The standard one-letter abbreviation for amino acids is used to illustrate the amino acid sequence.

FIGS. 2A-C show the polynucleotide sequence (SEQ ID NO:125) and deduced amino acid sequence (SEQ ID NO:126) of the NF-kB associated gene, AD037, of the present invention. The standard one-letter abbreviation for amino acids is used to illustrate the deduced amino acid sequence. The polynucleotide sequence contains a sequence of 2503 nucleotides (SEQ ID NO:125), encoding a polypeptide of 321 amino acids (SEQ ID NO:126). An analysis of the AD037 polypeptide determined that it comprised the following features: a Ras association motif located from about amino acid 172 to about amino acid 262 (SEQ ID NO:141) of SEQ ID NO:126 (FIGS. 2A-C) represented by shading; and three myrostylation sites located at amino acid 26-31, amino acid 102-107, and amino acid 186 to 191 of SEQ ID NO:126.

FIGS. 3A-B show the regions of identity and similarity between the encoded AD037 protein (SEQ ID NO:126) to the hypothetical protein KIAA0168, also referred to as the Ras association RalGDS/AF-6 domain family 2 protein (KIAA0168; Genbank Accession No. gil13274205; SEQ ID NO:129), the hypothetical mouse protein AK005472 (AK005472; Genbank Accession No. gil12838052; SEQ ID NO:130), and the Drosophila protein CG4656 (CG4656; Genbank Accession No. gil7300961; SEQ ID NO:131). The alignment was performed using the CLUSTALW algorithm using default parameters as described herein (Vector NTI suite of programs). The darkly shaded amino acids represent regions of matching identity. The lightly shaded amino acids represent regions of matching similarity. Dots (“●”) between residues indicate gapped regions of non-identity for the aligned polypeptides. The conserved cysteines between AD037 and the other proteins are noted.

FIG. 4 shows an expression profile of the NF-kB associated AD037 polypeptide (SEQ ID NO:126) that confirms the NF-kB-dependent regulation of AD037 expression. The figure illustrates the basal AD037 expression in unstimulated THP-1 monocytes and the observed increase in the relative AD037 expression level upon stimulation of the THP-1 monocytes with LPS. The figure also shows that the LPS-dependent AD037 expression is inhibited to near basal levels upon the administration of a selective NF-kB peptide inhibitor (SEQ ID NO:124). Expression data was obtained by measuring the steady state AD037 mRNA levels by quantitative PCR using the PCR primer pair provided as SEQ ID NO:162 and 163 as described herein.

FIG. 5 shows the level of secreted TNF-a recovered in the supernatant of THP-1 cells transfected with either “20 ug” or “10 ug” of pcDNA3.1mychis-AD037 expression vector after stimulation with 100 ng/ml LPS for 6 hours. As shown, the level of secreted TNF-a recovered was significantly inhibited in the presence of increased pcDNA3.1mychis-AD037 expression vector. The level of secreted TNF-a was determined using an ELISA assay as described herein.

FIG. 6 shows an expression profile of the NF-kB associated AD037 polypeptide in synovial samples derived from rheumatoid arthritis patients as compared to osteoarthritis synovium. As shown, the relative expression level of AD037 was signficantly increased in the synovia of rheumatoid arthritis patients. The expression data is consistent with AD037 being associated with NF-kB, and inflammatory disorders, in general. “NOR” refers to synovium samples derived from joint trauma controls; “OA” refers to synovial samples derived from osteoarthritis arthritis patients; and “RA” refers to synovial samples derived from rheumatoid arthritis patients. Expression data was obtained by measuring the steady state AD037 mRNA levels by quantitative PCR using the PCR primer pair provided as SEQ ID NO:162 and 163 as described herein.

FIG. 7 shows an expression profile of the NF-kB associated AD037 polypeptide (SEQ ID NO:126). The figure illustrates the relative expression level of AD037 amongst various mRNA tissue sources. As shown, transcripts corresponding to AD037 expressed predominately high in hematopoietic tissues including lymph node, spleen and leukocytes; signficantly in non-hematopoietic tissues including lung, pancreas, brain, kidney, and placenta, and to a lessser extent in heart, liver, thymus, tonsil, bone marrow, fetal liver, and skeletal muscle Expression data was obtained by measuring the steady state AD037 mRNA levels by quantitative PCR using the PCR primer pair provided as SEQ ID NO:162 and 163 as described herein.

FIG. 8 shows the results of a western blot using anti-Flag tag antibodies against lysates isolated from Cos7 cells transfected with the pcDNA3.1mychis-AD037 expression vector. As shown, a specific band of the expected size (approximately 40 kD) was detected in cells transfected with AD037 relative to cells transfected with vector alone. The Western blot was performed as described herein.

FIG. 9 shows confocal microscopic views of Cos7 cells transfected with pcDNA3.1mychis-AD037 expression vector after incubation with anti-Flag antibodies and FITC-labeled secondary antibodies. As shown, plasma membrane specific fluorescence was detected in cells transfected with AD037 (panel B), but not in cells transfected with vector alone (panel A). The results suggest AD037 associates with membrane-localized protein(s).

FIGS. 10A-H shows the polynucleotide and polypeptide sequences of proteins shown to interact with the AD037 polypeptide using a yeast two-hybrid screen. The full length AD037 was cloned into a bait vector that was used to screen a library derived from LPS-stimulated THP-1 cells. As shown, eight proteins were found to interact with AD037 and include the following: FEM-1b, the human homologue to C. elegans FEM-1 (Genbank Accession No: XM_{—}007581; SEQ ID NO:132 and 144); the human kinetochore protein CENP-H (Genbank Accession No: XM_{—}053172; SEQ ID NO:134 and 146); the human heat shock 70 kD protein (HSP70) (Genbank Accession No: XM_{—}050984; SEQ ID NO:135 and 147); the human large P1 ribosomal protein (Genbank Accession No: XM_{—}035389; SEQ ID NO:136 and 148); the human microtubule binding protein PAT1 (Genbank Accession No: XM_{—}018337; SEQ ID NO:137 and 149); the human BTB/POZ domain containing protein (Genbank Accession No: XM_{—}030647; SEQ ID NO:138 and 150); the human trinucleotide repeat containing 5 protein (Genbank Accession No: XM_{—}027629; SEQ ID NO:139 and 151); and the human FLJ12812 (Genbank Accession No: AK022874; SEQ ID NO:140 and 152). The start and stop codons of each polynucleotide are represented in bold.

FIGS. 11A-C show the polynucleotide sequence (SEQ ID NO:127) and deduced amino acid sequence (SEQ ID NO:128) of the NF-kB associated gene, Cyclin L, of the present invention. The standard one-letter abbreviation for amino acids is used to illustrate the deduced amino acid sequence. The polynucleotide sequence contains a sequence of 2076 nucleotides (SEQ ID NO:126), encoding a polypeptide of 526 amino acids (SEQ ID NO:128). An analysis of the Cyclin L polypeptide determined that it comprised the following features: a cyclin motif located from about amino acid 53 to about amino acid 197 (SEQ ID NO:142) of SEQ ID NO:128 (FIGS. 11A-C) represented by shading; and a factor TFIIB repeat sequence located from about amino acid 242 to about amino acid sequence 260 (SEQ ID NO:143) of SEQ ID NO:128 (FIGS. 11A-C) represented by single underlining.

FIGS. 12A-B show the regions of identity and similarity between the encoded Cyclin L protein (SEQ ID NO:128) to the rat cyclin L ortholog (Cyclin_L_Rat; Genbank Accession No. gil16758476; SEQ ID NO:153), the mouse cyclin L ortholog (Cyclin_L_Mou; Genbank Accession No. gil5453421; SEQ ID NO:154), the human protein AY037150 (AY037150; Genbank Accession No. gil14585859; SEQ ID NO:155), the Drosophila protein LD24704p (LD24704p; Genbank Accession No. gil16198007; SEQ ID NO:156), and the human cyclin T2b protein (Cyclin_T2b; Genbank Accession No. gil6691833; SEQ ID NO:157). The alignment was performed using the CLUSTALW algorithm using default parameters as described herein (Vector NTI suite of programs). The darkly shaded amino acids represent regions of matching identity. The lightly shaded amino acids represent regions of matching similarity. Dots (“●”) between residues indicate gapped regions of non-identity for the aligned polypeptides. The conserved cysteines between Cyclin L and the other proteins are noted.

FIG. 13 shows an expression profile of the NF-kB associated Cyclin L polypeptide (SEQ ID NO:128) that confirms the NF-kB-dependent regulation of Cyclin L expression. The figure illustrates the basal Cyclin L expression in unstimulated THP-1 monocytes and the observed increase in the relative Cyclin L expression level upon stimulation of the THP-1 monocytes with LPS. The figure also shows that the LPS-dependent Cyclin L expression is inhibited to near basal levels upon the administration of a selective NF-kB peptide inhibitor (SEQ ID NO:124). Expression data was obtained by measuring the steady state Cyclin L mRNA levels by quantitative PCR using the PCR primer pair provided as SEQ ID NO:164 and 165 as described herein.

FIG. 14 shows the level of secreted TNF-a recovered in the supernatant of THP-1 cells transfected with either “20 ug” or “10 ug” of pcDNA3.1mychis-Cyclin L expression vector after stimulation with 100 ng/ml LPS for 6 hours. As shown, the level of secreted TNF-a recovered was significantly inhibited in the presence of increased pcDNA3.1mychis-Cyclin L expression vector. The level of secreted TNF-a was determined using an ELISA assay as described herein.

FIG. 15 shows an expression profile of the NF-kB associated Cyclin L polypeptide (SEQ ID NO:128). The figure illustrates the relative expression level of Cyclin L amongst various mRNA tissue sources. As shown, transcripts corresponding to Cyclin L expressed predominately high in hematopoietic tissues including leukocytes, spleen, lymph node and thymus. Significant expression levels were detected in tonsil, bone marrow, and fetal liver. Expression data was obtained by measuring the steady state Cyclin L mRNA levels by quantitative PCR using the PCR primer pair provided as SEQ ID NO:164 and 165 as described herein.

FIGS. 16A-B shows the polynucleotide and polypeptide sequences of proteins shown to interact with the Cyclin L polypeptide using a yeast two-hybrid screen. The full length Cyclin L was cloned into a bait vector that was used to screen a library derived from LPS-stimulated THP-1 cells. As shown, two proteins were found to interact with Cyclin L and include the following: the human HSPC037 protein (Genbank Accession No: XM_{—}050490; SEQ ID NO:132 and 144); and the human heterogeneous nuclear ribonucleoprotein A2/B1 (Genbank Accession No: XM_{—}041353; SEQ ID NO:134 and 146). The start and stop codons of each polynucleotide are represented in bold.

FIG. 17 shows a table illustrating the percent identity and percent similarity between the NFkB associated polypeptides of the present invention to their closest homologs. The percent identity and percent similarity values were determined based upon the GAP algorithm (GCG suite of programs; and Henikoff, S. and Henikoff, J. G., Proc. Natl. Acad. Sci. USA 89: 10915-10919(1992)) using the following parameters: gap weight=8, and length weight=2.

FIG. 18 shows an expression profile of the NF-kB associated AD037 polypeptide (SEQ ID NO:126) in THP-1 human monocyte primary cell lines after stimulation with LPS, TNFα, or interferon-γ. The figure illustrates that AD037 mRNA is upregulated in response to stimuli that activate the NF-kB pathway including LPS and TNFα. As shown, little upregulation was observed in response to IFN-γ, which is with the AD037 being associated with the NF-kB pathway since IFN-gamma does not activate the NF-kB pathway. Expression data was obtained by measuring the steady state AD037 mRNA levels by quantitative PCR using the PCR primer pair provided as SEQ ID NO:162 and 163 as described herein.

FIG. 19 shows an expression profile of the NF-kB associated AD037 polypeptide (SEQ ID NO:126) in human peripheral blood neutrophil primary cell lines isolated from two different donors that had been stimulated for 24 or 48 hours with LPS. The figure illustrates that AD037 mRNA is upregulated in response to LPS stimuli which is consistent with its association with the NF-kB pathway. Expression data was obtained by measuring the steady state AD037 mRNA levels by quantitative PCR using the PCR primer pair provided as SEQ ID NO:162 and 163 as described herein.

FIG. 20 shows an expression profile of the NF-kB associated AD037 polypeptide (SEQ ID NO:126) in human synovial fibroblast primary cell lines after stimulation with either TNFα, IL-1α, IL-17, or an IL-17B-Ig fusion protein for 1, 6, or 24 hours. The figure illustrates that AD037 mRNA is selectively upregulated in response to IL-17B. Expression data was obtained by measuring the steady state AD037 mRNA levels by quantitative PCR using the PCR primer pair provided as SEQ ID NO:162 and 163 as described herein.

FIG. 21 shows an expression profile of the NF-kB associated AD037 polypeptide (SEQ ID NO:126) in human peripheral blood B cell lines after stimulation with anti-CD40 antibody for either 6 or 24 hours. The figure illustrates that AD037 mRNA is upregulated in response to CD40 crosslinking, which is also consistent with its association with the NF-kB pathway. Expression data was obtained by measuring the steady state AD037 mRNA levels by quantitative PCR using the PCR primer pair provided as SEQ ID NO:162 and 163 as described herein.

FIG. 22 shows an expression profile of the NF-kB associated AC008435 polypeptide using primers specific to its encoding polynucleotide or portions thereof (SEQ ID NO:7, and SEQ ID NO:264) that confirms the NF-kB-dependent regulation of AC008435 expression. The figure illustrates the basal AC008435 expression in unstimulated THP-1 monocytes and the observed increase in the relative AC008435 expression level upon stimulation of the THP-1 monocytes with LPS. The figure also shows that the LPS-dependent AC008435 expression is inhibited to near basal levels upon the administration of a selective NF-kB peptide inhibitor (SEQ ID NO:124). Expression data was obtained by measuring the steady state AC008435 mRNA levels by quantitative PCR using the PCR primer pair provided as SEQ ID NO:210 and 211 as described herein.

FIG. 23 shows an expression profile of the NF-kB associated AC008435 polypeptide using primers specific to its encoding polynucleotide or portions thereof (SEQ ID NO:7, and SEQ ID NO:264). The figure illustrates the relative expression level of AC008435 amongst various mRNA tissue sources. Expression data was obtained by measuring the steady state AC008435 mRNA levels by quantitative PCR using the PCR primer pair provided as SEQ ID NO:210 and 211 as described herein.

FIG. 24 shows an expression profile of the NF-kB associated AC005625 polypeptide using primers specific to its encoding polynucleotide or portions thereof (SEQ ID NO:8) that confirms the NF-kB-dependent regulation of AC005625 expression. The figure illustrates the basal AC005625 expression in unstimulated THP-1 monocytes and the observed increase in the relative AC005625 expression level upon stimulation of the THP-1 monocytes with LPS. The figure also shows that the LPS-dependent AC005625 expression is inhibited to near basal levels upon the administration of a selective NF-kB peptide inhibitor (SEQ ID NO:124). Expression data was obtained by measuring the steady state AC005625 mRNA levels by quantitative PCR using the PCR primer pair provided as SEQ ID NO:234 and 235 as described herein.

FIG. 25 shows an expression profile of the NF-kB associated AC005625 polypeptide using primers specific to its encoding polynucleotide or portions thereof (SEQ ID NO:8). The figure illustrates the relative expression level of AC005625 amongst various mRNA tissue sources. Expression data was obtained by measuring the steady state AC005625 mRNA levels by quantitative PCR using the PCR primer pair provided as SEQ ID NO:234 and 235 as described herein.

FIG. 26 shows an expression profile of the NF-kB associated AL354881 polypeptide using primers specific to its encoding polynucleotide or portions thereof (SEQ ID NO:9, and SEQ ID NO:265) that confirms the NF-kB-dependent regulation of AL354881 expression. The figure illustrates the basal AL354881 expression in unstimulated THP-1 monocytes and the observed increase in the relative AL354881 expression level upon stimulation of the THP-1 monocytes with LPS. The figure also shows that the LPS-dependent AL354881 expression is inhibited to near basal levels upon the administration of a selective NF-kB peptide inhibitor (SEQ ID NO:124). Expression data was obtained by measuring the steady state AL354881 mRNA levels by quantitative PCR using the PCR primer pair provided as SEQ ID NO:216 and 217 as described herein.

FIG. 27 shows an expression profile of the NF-kB associated AL354881 polypeptide using primers specific to its encoding polynucleotide or portions thereof (SEQ ID NO:9, and SEQ ID NO:265). The figure illustrates the relative expression level of AL354881 amongst various mRNA tissue sources. Expression data was obtained by measuring the steady state AL354881 mRNA levels by quantitative PCR using the PCR primer pair provided as SEQ ID NO:216 and 217 as described herein.

FIG. 28 shows an expression profile of the NF-kB associated AC008576 polypeptide using primers specific to its encoding polynucleotide or portions thereof (SEQ ID NO:21) that confirms the NF-kB-dependent regulation of AC008576 expression. The figure illustrates the basal AC008576 expression in unstimulated THP-1 monocytes and the observed increase in the relative AC008576 expression level upon stimulation of the THP-1 monocytes with LPS. The figure also shows that the LPS-dependent AC008576 expression is inhibited to near basal levels upon the administration of a selective NF-kB peptide inhibitor (SEQ ID NO:124). Expression data was obtained by measuring the steady state AC008576 mRNA levels by quantitative PCR using the PCR primer pair provided as SEQ ID NO:242 and 243 as described herein.

FIG. 29 shows an expression profile of the NF-kB associated AC008576 polypeptide using primers specific to its encoding polynucleotide or portions thereof (SEQ ID NO:21). The figure illustrates the relative expression level of AC008576 amongst various mRNA tissue sources. Expression data was obtained by measuring the steady state AC008576 mRNA levels by quantitative PCR using the PCR primer pair provided as SEQ ID NO:242 and 243 as described herein.

FIG. 30 shows an expression profile of the NF-kB associated AC023602 polypeptide using primers specific to its encoding polynucleotide or portions thereof (SEQ ID NO:14, and SEQ ID NO:266) that confirms the NF-kB-dependent regulation of AC023602 expression. The figure illustrates the basal AC023602 expression in unstimulated THP-1 monocytes and the observed increase in the relative AC023602 expression level upon stimulation of the THP-1 monocytes with LPS. The figure also shows that the LPS-dependent AC023602 expression is inhibited to near basal levels upon the administration of a selective NF-kB peptide inhibitor (SEQ ID NO:124). Expression data was obtained by measuring the steady state AC023602 mRNA levels by quantitative PCR using the PCR primer pair provided as SEQ ID NO:240 and 241 as described herein.

FIG. 31 shows an expression profile of the NF-kB associated AC023602 polypeptide using primers specific to its encoding polynucleotide or portions thereof (SEQ ID NO:14, and SEQ ID NO:266). The figure illustrates the relative expression level of AC023602 amongst various mRNA tissue sources. Expression data was obtained by measuring the steady state AC023602 mRNA levels by quantitative PCR using the PCR primer pair provided as SEQ ID NO:240 and 241 as described herein.

FIG. 32 shows an expression profile of the NF-kB associated AL136163 polypeptide using primers specific to its encoding polynucleotide or portions thereof (SEQ ID NO:22) that confirms the NF-kB-dependent regulation of AL136163 expression. The figure illustrates the basal AL136163 expression in unstimulated THP-1 monocytes and the observed increase in the relative AL136163 expression level upon stimulation of the THP-1 monocytes with LPS. The figure also shows that the LPS-dependent AL136163 expression is inhibited to near basal levels upon the administration of a selective NF-kB peptide inhibitor (SEQ ID NO:124). Expression data was obtained by measuring the steady state AL136163 mRNA levels by quantitative PCR using the PCR primer pair provided as SEQ ID NO:208 and 209 as described herein.

FIG. 33 shows an expression profile of the NF-kB associated AL136163 polypeptide using primers specific to its encoding polynucleotide or portions thereof (SEQ ID NO:22). The figure illustrates the relative expression level of AL136163 amongst various mRNA tissue sources. Expression data was obtained by measuring the steady state AL136163 mRNA levels by quantitative PCR using the PCR primer pair provided as SEQ ID NO:208 and 209 as described herein.

FIG. 34 shows an expression profile of the NF-kB associated AP002338 polypeptide using primers specific to its encoding polynucleotide or portions thereof (SEQ ID NO:27, and SEQ ID NO:267) that confirms the NF-kB-dependent regulation of AP002338 expression. The figure illustrates the basal AP002338 expression in unstimulated THP-1 monocytes and the observed increase in the relative AP002338 expression level upon stimulation of the THP-1 monocytes with LPS. The figure also shows that the LPS-dependent AP002338 expression is inhibited to near basal levels upon the administration of a selective NF-kB peptide inhibitor (SEQ ID NO:124). Expression data was obtained by measuring the steady state AP002338 mRNA levels by quantitative PCR using the PCR primer pair provided as SEQ ID NO:206 and 207 as described herein.

FIG. 35 shows an expression profile of the NF-kB associated AP002338 polypeptide using primers specific to its encoding polynucleotide or portions thereof (SEQ ID NO:27, and SEQ ID NO:267). The figure illustrates the relative expression level of AP002338 amongst various mRNA tissue sources. Expression data was obtained by measuring the steady state AP002338 mRNA levels by quantitative PCR using the PCR primer pair provided as SEQ ID NO:206 and 207 as described herein.

FIG. 36 shows an expression profile of the NF-kB associated AL158062 polypeptide using primers specific to its encoding polynucleotide or portions thereof (SEQ ID NO:28, and SEQ ID NO:268) that confirms the NF-kB-dependent regulation of AL158062 expression. The figure illustrates the basal AL158062 expression in unstimulated THP-1 monocytes and the observed increase in the relative AL158062 expression level upon stimulation of the THP-1 monocytes with LPS. The figure also shows that the LPS-dependent AL158062 expression is inhibited to near basal levels upon the administration of a selective NF-kB peptide inhibitor (SEQ ID NO:124). Expression data was obtained by measuring the steady state AL158062 mRNA levels by quantitative PCR using the PCR primer pair provided as SEQ ID NO:244 and 245 as described herein.

FIG. 37 shows an expression profile of the NF-kB associated AL158062 polypeptide using primers specific to its encoding polynucleotide or portions thereof (SEQ ID NO:28, and SEQ ID NO:268). The figure illustrates the relative expression level of AL158062 amongst various mRNA tissue sources. Expression data was obtained by measuring the steady state AL158062 mRNA levels by quantitative PCR using the PCR primer pair provided as SEQ ID NO:244 and 245 as described herein.

FIG. 38 shows an expression profile of the NF-kB associated AC015564 polypeptide using primers specific to its encoding polynucleotide or portions thereof (SEQ ID NO:33, and SEQ ID NO:269) that confirms the NF-kB-dependent regulation of AC015564 expression. The figure illustrates the basal AC015564 expression in unstimulated THP-1 monocytes and the observed increase in the relative AC015564 expression level upon stimulation of the THP-1 monocytes with LPS. The figure also shows that the LPS-dependent AC015564 expression is inhibited to near basal levels upon the administration of a selective NF-kB peptide inhibitor (SEQ ID NO:124). Expression data was obtained by measuring the steady state AC015564 mRNA levels by quantitative PCR using the PCR primer pair provided as SEQ ID NO:224 and 225 as described herein.

FIG. 39 shows an expression profile of the NF-kB associated AC015564 polypeptide using primers specific to its encoding polynucleotide or portions thereof (SEQ ID NO:33, and SEQ ID NO:269). The figure illustrates the relative expression level of AC015564 amongst various mRNA tissue sources. Expression data was obtained by measuring the steady state AC015564 mRNA levels by quantitative PCR using the PCR primer pair provided as SEQ ID NO:224 and 225 as described herein.

FIG. 40 shows an expression profile of the NF-kB associated 116917 polypeptide using primers specific to its encoding polynucleotide or portions thereof (SEQ ID NO:36, and SEQ ID NO:270) that confirms the NF-kB-dependent regulation of 116917 expression. The figure illustrates the basal 116917 expression in unstimulated THP-1 monocytes and the observed increase in the relative 116917 expression level upon stimulation of the THP-1 monocytes with LPS. The figure also shows that the LPS-dependent 116917 expression is inhibited to near basal levels upon the administration of a selective NF-kB peptide inhibitor (SEQ ID NO:124). Expression data was obtained by measuring the steady state 116917 mRNA levels by quantitative PCR using the PCR primer pair provided as SEQ ID NO:246 and 247 as described herein.

FIG. 41 shows an expression profile of the NF-kB associated 116917 polypeptide using primers specific to its encoding polynucleotide or portions thereof (SEQ ID NO:36, and SEQ ID NO:270). The figure illustrates the relative expression level of 116917 amongst various mRNA tissue sources. Expression data was obtained by measuring the steady state 116917 mRNA levels by quantitative PCR using the PCR primer pair provided as SEQ ID NO:246 and 247 as described herein.

FIG. 42 shows an expression profile of the NF-kB associated 1137189 polypeptide using primers specific to its encoding polynucleotide or portions thereof (SEQ ID NO:39, and SEQ ID NO:271) that confirms the NF-kB-dependent regulation of 1137189 expression. The figure illustrates the basal 1137189 expression in unstimulated THP-1 monocytes and the observed increase in the relative 1137189 expression level upon stimulation of the THP-1 monocytes with LPS. The figure also shows that the LPS-dependent 1137189 expression is inhibited to near basal levels upon the administration of a selective NF-kB peptide inhibitor (SEQ ID NO:124). Expression data was obtained by measuring the steady state 1137189 mRNA levels by quantitative PCR using the PCR primer pair provided as SEQ ID NO:248 and 249 as described herein.

FIG. 43 shows an expression profile of the NF-kB associated 1137189 polypeptide using primers specific to its encoding polynucleotide or portions thereof (SEQ ID NO:39, and SEQ ID NO:271). The figure illustrates the relative expression level of 1137189 amongst various mRNA tissue sources. Expression data was obtained by measuring the steady state 1137189 mRNA levels by quantitative PCR using the PCR primer pair provided as SEQ ID NO:248 and 249 as described herein.

FIG. 44 shows an expression profile of the NF-kB associated 899587 polypeptide using primers specific to its encoding polynucleotide or portions thereof (SEQ ID NO:46, and SEQ ID NO:272) that confirms the NF-kB-dependent regulation of 899587 expression. The figure illustrates the basal 899587 expression in unstimulated THP-1 monocytes and the observed increase in the relative 899587 expression level upon stimulation of the THP-1 monocytes with LPS. The figure also shows that the LPS-dependent 899587 expression is inhibited to near basal levels upon the administration of a selective NF-kB peptide inhibitor (SEQ ID NO:124). Expression data was obtained by measuring the steady state 899587 mRNA levels by quantitative PCR using the PCR primer pair provided as SEQ ID NO:250 and 251 as described herein.

FIG. 45 shows an expression profile of the NF-kB associated 899587 polypeptide using primers specific to its encoding polynucleotide or portions thereof (SEQ ID NO:46, and SEQ ID NO:272). The figure illustrates the relative expression level of 899587 amongst various mRNA tissue sources. Expression data was obtained by measuring the steady state 899587 mRNA levels by quantitative PCR using the PCR primer pair provided as SEQ ID NO:250 and 251 as described herein.

FIG. 46 shows an expression profile of the NF-kB associated 337323 polypeptide using primers specific to its encoding polynucleotide or portions thereof (SEQ ID NO:50, and SEQ ID NO:273) that confirms the NF-kB-dependent regulation of 337323 expression. The figure illustrates the basal 337323 expression in unstimulated THP-1 monocytes and the observed increase in the relative 337323 expression level upon stimulation of the THP-1 monocytes with LPS. The figure also shows that the LPS-dependent 337323 expression is inhibited to near basal levels upon the administration of a selective NF-kB peptide inhibitor (SEQ ID NO:124). Expression data was obtained by measuring the steady state 337323 mRNA levels by quantitative PCR using the PCR primer pair provided as SEQ ID NO:214 and 215 as described herein.

FIG. 47 shows an expression profile of the NF-kB associated 337323 polypeptide using primers specific to its encoding polynucleotide or portions thereof (SEQ ID NO:50, and SEQ ID NO:273). The figure illustrates the relative expression level of 337323 amongst various mRNA tissue sources. Expression data was obtained by measuring the steady state 337323 mRNA levels by quantitative PCR using the PCR primer pair provided as SEQ ID NO:214 and 215 as described herein.

FIG. 48 shows an expression profile of the NF-kB associated 346607 polypeptide using primers specific to its encoding polynucleotide or portions thereof (SEQ ID NO:52, and SEQ ID NO:274) that confirms the NF-kB-dependent regulation of 346607 expression. The figure illustrates the basal 346607 expression in unstimulated THP-1 monocytes and the observed increase in the relative 346607 expression level upon stimulation of the THP-1 monocytes with LPS. The figure also shows that the LPS-dependent 346607 expression is inhibited to near basal levels upon the administration of a selective NF-kB peptide inhibitor (SEQ ID NO:124). Expression data was obtained by measuring the steady state 346607 mRNA levels by quantitative PCR using the PCR primer pair provided as SEQ ID NO:212 and 213 as described herein.

FIG. 49 shows an expression profile of the NF-kB associated 346607 polypeptide using primers specific to its encoding polynucleotide or portions thereof (SEQ ID NO:52, and SEQ ID NO:274). The figure illustrates the relative expression level of 346607 amongst various mRNA tissue sources. Expression data was obtained by measuring the steady state 346607 mRNA levels by quantitative PCR using the PCR primer pair provided as SEQ ID NO:212 and 213 as described herein.

FIG. 50 shows an expression profile of the NF-kB associated 404343 polypeptide using primers specific to its encoding polynucleotide or portions thereof (SEQ ID NO:56, and SEQ ID NO:275) that confirms the NF-kB-dependent regulation of 404343 expression. The figure illustrates the basal 404343 expression in unstimulated THP-1 monocytes and the observed increase in the relative 404343 expression level upon stimulation of the THP-1 monocytes with LPS. The figure also shows that the LPS-dependent 404343 expression is inhibited to near basal levels upon the administration of a selective NF-kB peptide inhibitor (SEQ ID NO:124). Expression data was obtained by measuring the steady state 404343 mRNA levels by quantitative PCR using the PCR primer pair provided as SEQ ID NO:222 and 223 as described herein.

FIG. 51 shows an expression profile of the NF-kB associated 404343 polypeptide using primers specific to its encoding polynucleotide or portions thereof (SEQ ID NO:56, and SEQ ID NO:275). The figure illustrates the relative expression level of 404343 amongst various mRNA tissue sources. Expression data was obtained by measuring the steady state 404343 mRNA levels by quantitative PCR using the PCR primer pair provided as SEQ ID NO:222 and 223 as described herein.

FIG. 52 shows an expression profile of the NF-kB associated 30507 polypeptide using primers specific to its encoding polynucleotide or portions thereof (SEQ ID NO:57, and SEQ ID NO:276) that confirms the NF-kB-dependent regulation of 30507 expression. The figure illustrates the basal 30507 expression in unstimulated THP-1 monocytes and the observed increase in the relative 30507 expression level upon stimulation of the THP-1 monocytes with LPS. The figure also shows that the LPS-dependent 30507 expression is inhibited to near basal levels upon the administration of a selective NF-kB peptide inhibitor (SEQ ID NO:124). Expression data was obtained by measuring the steady state 30507 mRNA levels by quantitative PCR using the PCR primer pair provided as SEQ ID NO:252 and 253 as described herein.

FIG. 53 shows an expression profile of the NF-kB associated 30507 polypeptide using primers specific to its encoding polynucleotide or portions thereof (SEQ ID NO:57, and SEQ ID NO:276). The figure illustrates the relative expression level of 30507 amongst various mRNA tissue sources. Expression data was obtained by measuring the steady state 30507 mRNA levels by quantitative PCR using the PCR primer pair provided as SEQ ID NO:252 and 253 as described herein.

FIG. 54 shows an expression profile of the NF-kB associated 242250 polypeptide using primers specific to its encoding polynucleotide or portions thereof (SEQ ID NO:70, and SEQ ID NO:277) that confirms the NF-kB-dependent regulation of 242250 expression. The figure illustrates the basal 242250 expression in unstimulated THP-1 monocytes and the observed increase in the relative 242250 expression level upon stimulation of the THP-1 monocytes with LPS. The figure also shows that the LPS-dependent 242250 expression is inhibited to near basal levels upon the administration of a selective NF-kB peptide inhibitor (SEQ ID NO:124). Expression data was obtained by measuring the steady state 242250 mRNA levels by quantitative PCR using the PCR primer pair provided as SEQ ID NO:226 and 227 as described herein.

FIG. 55 shows an expression profile of the NF-kB associated 242250 polypeptide using primers specific to its encoding polynucleotide or portions thereof (SEQ ID NO:70, and SEQ ID NO:277). The figure illustrates the relative expression level of 242250 amongst various mRNA tissue sources. Expression data was obtained by measuring the steady state 242250 mRNA levels by quantitative PCR using the PCR primer pair provided as SEQ ID NO:226 and 227 as described herein.

FIG. 56 shows an expression profile of the NF-kB associated 262 polypeptide using primers specific to its encoding polynucleotide or portions thereof (SEQ ID NO:92, and SEQ ID NO:262) that confirms the NF-kB-dependent regulation of 262 expression. The figure illustrates the basal 262 expression in unstimulated THP-1 monocytes and the observed increase in the relative 262 expression level upon stimulation of the THP-1 monocytes with LPS. The figure also shows that the LPS-dependent 262 expression is inhibited to near basal levels upon the administration of a selective NF-kB peptide inhibitor (SEQ ID NO:124). Expression data was obtained by measuring the steady state 262 mRNA levels by quantitative PCR using the PCR primer pair provided as SEQ ID NO:262 and 263 as described herein.

FIG. 57 shows an expression profile of the NF-kB associated 262 polypeptide using primers specific to its encoding polynucleotide or portions thereof (SEQ ID NO:92, and SEQ ID NO:262). The figure illustrates the relative expression level of 262 amongst various mRNA tissue sources. Expression data was obtained by measuring the steady state 262 mRNA levels by quantitative PCR using the PCR primer pair provided as SEQ ID NO:262 and 263 as described herein.

FIG. 58 shows an expression profile of the NF-kB associated 360 polypeptide using primers specific to its encoding polynucleotide or portions thereof (SEQ ID NO:97) that confirms the NF-kB-dependent regulation of 360 expression. The figure illustrates the basal 360 expression in unstimulated THP-1 monocytes and the observed increase in the relative 360 expression level upon stimulation of the THP-1 monocytes with LPS. The figure also shows that the LPS-dependent 360 expression is inhibited to near basal levels upon the administration of a selective NF-kB peptide inhibitor (SEQ ID NO:124). Expression data was obtained by measuring the steady state 360 mRNA levels by quantitative PCR using the PCR primer pair provided as SEQ ID NO:258 and 259 as described herein.

FIG. 59 shows an expression profile of the NF-kB associated 360 polypeptide using primers specific to its encoding polynucleotide or portions thereof (SEQ ID NO:97). The figure illustrates the relative expression level of 360 amongst various mRNA tissue sources. Expression data was obtained by measuring the steady state 360 mRNA levels by quantitative PCR using the PCR primer pair provided as SEQ ID NO:258 and 259 as described herein.

FIG. 60 shows an expression profile of the NF-kB associated AC025631 polypeptide using primers specific to its encoding polynucleotide or portions thereof (SEQ ID NO:101) that confirms the NF-kB-dependent regulation of AC025631 expression. The figure illustrates the basal AC025631 expression in unstimulated THP-1 monocytes and the observed increase in the relative AC025631 expression level upon stimulation of the THP-1 monocytes with LPS. The figure also shows that the LPS-dependent AC025631 expression is inhibited to near basal levels upon the administration of a selective NF-kB peptide inhibitor (SEQ ID NO:124). Expression data was obtained by measuring the steady state AC025631 mRNA levels by quantitative PCR using the PCR primer pair provided as SEQ ID NO:260 and 261 as described herein.

FIG. 61 shows an expression profile of the NF-kB associated AC025631 polypeptide using primers specific to its encoding polynucleotide or portions thereof (SEQ ID NO:101). The figure illustrates the relative expression level of AC025631 amongst various mRNA tissue sources. Expression data was obtained by measuring the steady state AC025631 mRNA levels by quantitative PCR using the PCR primer pair provided as SEQ ID NO:260 and 261 as described herein.

FIG. 62 shows an expression profile of the NF-kB associated 7248 polypeptide using primers specific to its encoding polynucleotide or portions thereof (SEQ ID NO:40, and SEQ ID NO:279) that confirms the NF-kB-dependent regulation of 7248 expression. The figure illustrates the basal 7248 expression in unstimulated THP-1 monocytes and the observed increase in the relative 7248 expression level upon stimulation of the THP-1 monocytes with LPS. The figure also shows that the LPS-dependent 7248 expression is inhibited to near basal levels upon the administration of a selective NF-kB peptide inhibitor (SEQ ID NO:124). Expression data was obtained by measuring the steady state 7248 mRNA levels by quantitative PCR using the PCR primer pair provided as SEQ ID NO:220 and 221 as described herein.

FIG. 63 shows an expression profile of the NF-kB associated 7248 polypeptide using primers specific to its encoding polynucleotide or portions thereof (SEQ ID NO:40, and SEQ ID NO:279). The figure illustrates the relative expression level of 7248 amongst various mRNA tissue sources. Expression data was obtained by measuring the steady state 7248 mRNA levels by quantitative PCR using the PCR primer pair provided as SEQ ID NO:220 and 221 as described herein.

FIG. 64 shows an expression profile of the NF-kB associated 127 polypeptide using primers specific to its encoding polynucleotide or portions thereof (SEQ ID NO:102) that confirms the NF-kB-dependent regulation of 127 expression. The figure illustrates the basal 127 expression in unstimulated THP-1 monocytes and the observed increase in the relative 127 expression level upon stimulation of the THP-1 monocytes with LPS. The figure also shows that the LPS-dependent 127 expression is inhibited to near basal levels upon the administration of a selective NF-kB peptide inhibitor (SEQ ID NO:124). Expression data was obtained by measuring the steady state 127 mRNA levels by quantitative PCR using the PCR primer pair provided as SEQ ID NO:218 and 219 as described herein.

FIG. 65 shows an expression profile of the NF-kB associated 127 polypeptide using primers specific to its encoding polynucleotide or portions thereof (SEQ ID NO:102). The figure illustrates the relative expression level of 127 amongst various mRNA tissue sources. Expression data was obtained by measuring the steady state 127 mRNA levels by quantitative PCR using the PCR primer pair provided as SEQ ID NO:218 and 219 as described herein.

FIG. 66 shows an expression profile of the NF-kB associated AC007014 polypeptide using primers specific to its encoding polynucleotide or portions thereof (SEQ ID NO:10, and SEQ ID NO:280) that confirms the NF-kB-dependent regulation of AC007014 expression. The figure illustrates the basal AC007014 expression in unstimulated THP-1 monocytes and the observed decrease in the relative AC007014 expression level upon stimulation of the THP-1 monocytes with LPS. The figure also shows that the LPS-dependent AC007014 expression is brought back to near basal levels upon the administration of a selective NF-kB peptide inhibitor (SEQ ID NO:124). Expression data was obtained by measuring the steady state AC007014 mRNA levels by quantitative PCR using the PCR primer pair provided as SEQ ID NO:236 and 237 as described herein.

FIG. 67 shows an expression profile of the NF-kB associated AC010791 polypeptide using primers specific to its encoding polynucleotide or portions thereof (SEQ ID NO:I 1, and SEQ ID NO:28 1) that confirms the NF-kB-dependent regulation of AC010791 expression. The figure illustrates the basal AC010791 expression in unstimulated THP-1 monocytes and the observed decrease in the relative AC010791 expression level upon stimulation of the THP-1 monocytes with LPS. The figure also shows that the LPS-dependent AC010791 expression is brought back to near basal levels upon the administration of a selective NF-kB peptide inhibitor (SEQ ID NO:124). Expression data was obtained by measuring the steady state AC010791 mRNA levels by quantitative PCR using the PCR primer pair provided as SEQ ID NO:238 and 239 as described herein.

FIG. 68 shows an expression profile of the NF-kB associated AC010791 polypeptide using primers specific to its encoding polynucleotide or portions thereof (SEQ ID NO:11, and SEQ ID NO:281). The figure illustrates the relative expression level of AC010791 amongst various mRNA tissue sources. Expression data was obtained by measuring the steady state AC010791 mRNA levels by quantitative PCR using the PCR primer pair provided as SEQ ID NO:238 and 239 as described herein.

FIG. 69 shows an expression profile of the NF-kB associated AC040977 polypeptide using primers specific to its encoding polynucleotide or portions thereof (SEQ ID NO:62) that confirms the NF-kB-dependent regulation of AC040977 expression. The figure illustrates the basal AC040977 expression in unstimulated THP-1 monocytes and the observed decrease in the relative AC040977 expression level upon stimulation of the THP-1 monocytes with LPS. The figure also shows that the LPS-dependent AC040977 expression is brought back to near basal levels upon the administration of a selective NF-kB peptide inhibitor (SEQ ID NO:124). Expression data was obtained by measuring the steady state AC040977 mRNA levels by quantitative PCR using the PCR primer pair provided as SEQ ID NO:254 and 255 as described herein.

FIG. 70 shows an expression profile of the NF-kB associated AC040977 polypeptide using primers specific to its encoding polynucleotide or portions thereof (SEQ ID NO:62). The figure illustrates the relative expression level of AC040977 amongst various mRNA tissue sources. Expression data was obtained by measuring the steady state AC040977 mRNA levels by quantitative PCR using the PCR primer pair provided as SEQ ID NO:254 and 255 as described herein.

FIG. 71 shows an expression profile of the NF-kB associated AC012357 polypeptide using primers specific to its encoding polynucleotide or portions thereof (SEQ ID NO:68) that confirms the NF-kB-dependent regulation of AC012357 expression. The figure illustrates the basal AC012357 expression in unstimulated THP-1 monocytes and the observed decrease in the relative AC012357 expression level upon stimulation of the THP-1 monocytes with LPS. The figure also shows that the LPS-dependent AC012357 expression is brought back to near basal levels upon the administration of a selective NF-kB peptide inhibitor (SEQ ID NO:124). Expression data was obtained by measuring the steady state AC012357 mRNA levels by quantitative PCR using the PCR primer pair provided as SEQ ID NO:256 and 257 as described herein.

FIG. 72 shows an expression profile of the NF-kB associated AC012357 polypeptide using primers specific to its encoding polynucleotide or portions thereof (SEQ ID NO:68). The figure illustrates the relative expression level of AC012357 amongst various mRNA tissue sources. Expression data was obtained by measuring the steady state AC012357 mRNA levels by quantitative PCR using the PCR primer pair provided as SEQ ID NO:256 and 257 as described herein.

FIG. 73 shows an expression profile of the NF-kB associated AC024191 polypeptide using primers specific to its encoding polynucleotide or portions thereof (SEQ ID NO:74, and SEQ ID NO:284) that confirms the NF-kB-dependent regulation of AC024191 expression. The figure illustrates the basal AC024191 expression in unstimulated THP-1 monocytes and the observed decrease in the relative AC024191 expression level upon stimulation of the THP-1 monocytes with LPS. The figure also shows that the LPS-dependent AC024191 expression is brought back to near basal levels upon the administration of a selective NF-kB peptide inhibitor (SEQ ID NO:124). Expression data was obtained by measuring the steady state AC024191 mRNA levels by quantitative PCR using the PCR primer pair provided as SEQ ID NO:228 and 229 as described herein.

FIG. 74 shows an expression profile of the NF-kB associated AC024191 polypeptide using primers specific to its encoding polynucleotide or portions thereof (SEQ ID NO:74, and SEQ ID NO:284). The figure illustrates the relative expression level of AC024191 amongst various mRNA tissue sources. Expression data was obtained by measuring the steady state AC024191 mRNA levels by quantitative PCR using the PCR primer pair provided as SEQ ID NO:228 and 229 as described herein.

FIG. 75 shows an expression profile of the NF-kB associated 235347 polypeptide using primers specific to its encoding polynucleotide or portions thereof (SEQ ID NO:78, and SEQ ID NO:282) that confirms the NF-kB-dependent regulation of 235347 expression. The figure illustrates the basal 235347 expression in unstimulated THP-1 monocytes and the observed decrease in the relative 235347 expression level upon stimulation of the THP-1 monocytes with LPS. The figure also shows that the LPS-dependent 235347 expression is brought back to near basal levels upon the administration of a selective NF-kB peptide inhibitor (SEQ ID NO:124). Expression data was obtained by measuring the steady state 235347 mRNA levels by quantitative PCR using the PCR primer pair provided as SEQ ID NO:232 and 233 as described herein.

FIG. 76 shows an expression profile of the NF-kB associated 235347 polypeptide using primers specific to its encoding polynucleotide or portions thereof (SEQ ID NO:78, and SEQ ID NO:282). The figure illustrates the relative expression level of 235347 amongst various mRNA tissue sources. Expression data was obtained by measuring the steady state 235347 mRNA levels by quantitative PCR using the PCR primer pair provided as SEQ ID NO:232 and 233 as described herein.

FIG. 77 shows an expression profile of the NF-kB associated 204305 polypeptide using primers specific to its encoding polynucleotide or portions thereof (SEQ ID NO:81) that confirms the NF-kB-dependent regulation of 204305 expression. The figure illustrates the basal 204305 expression in unstimulated THP-1 monocytes and the observed decrease in the relative 204305 expression level upon stimulation of the THP-1 monocytes with LPS. The figure also shows that the LPS-dependent 204305 expression is brought back to near basal levels upon the administration of a selective NF-kB peptide inhibitor (SEQ ID NO:124). Expression data was obtained by measuring the steady state 204305 mRNA levels by quantitative PCR using the PCR primer pair provided as SEQ ID NO:230 and 231 as described herein.

FIG. 78 shows an expression profile of the NF-kB associated 204305 polypeptide using primers specific to its encoding polynucleotide or portions thereof (SEQ ID NO:81). The figure illustrates the relative expression level of 204305 amongst various mRNA tissue sources. Expression data was obtained by measuring the steady state 204305 mRNA levels by quantitative PCR using the PCR primer pair provided as SEQ ID NO:230 and 231 as described herein.

FIG. 79 shows the results of a microarray profile of the NF-kB associated 36d5, 37e4, 42e7, 105b2, and 41h1 that confirms the NF-kB-dependent regulation of 36d5, 37e4, 42e7, 105b2, and 41h1 expression. The figure illustrates the basal 36d5, 37e4, 42e7, 105b2, and 41h1 expression in unstimulated THP-1 monocytes and the observed increase in the relative 36d5, 37e4, 42e7, 105b2, and 41h1 expression level upon stimulation of the THP-1 monocytes with LPS. The figure also shows that the LPS-dependent 36d5, 37e4, 42e7, 105b2, and 41h1 expression is inhibited to near basal levels upon the administration of a selective NF-kB peptide inhibitor (SEQ ID NO:124).

FIG. 80 shows an expression profile of the NF-kB associated AD037 polypeptide using primers specific to its encoding polynucleotide or portions thereof (SEQ ID NO:126) that further confirms the NF-kB-dependent regulation of AD037 expression. The figure illustrates the basal AD037 expression in THP-1 monocytes in response to LPS (“LPS”), LPS and the glucocorticoid dexamethasone (“LP/Dex”), or LPS and the IKK-2 inhibitor BMS-345541 (“LPS/345541”), for 2 hours, 4 hours, and 8 afters post stimulation. Unstimulated THP-1 moncytes served as a control. As shown, AD037 expression was significantly induced upon stimulation with LPS and LPS/Dex, with the latter resulting in the highest level of induction. The increase in LPS-induced AD037 expression was reduced to control levels upon incubation with LPS/345541. Expression data was obtained by measuring the steady state AD037 mRNA levels by quantitative PCR using the PCR primer pair provided as SEQ ID NO:162 and 163 as described herein.

FIG. 81 shows an expression profile of the NF-kB associated AD037 polypeptide using primers specific to its encoding polynucleotide or portions thereof (SEQ ID NO:126) that further confirms the NF-kB-dependent regulation of AD037 expression. The figure illustrates the basal AD037 expression in mouse embryonic fibroblasts derived from germline knockouts of different NF-κB family members, specifically mouse embryonic germline knockouts of p65, RelB, and p50 upon stimulation for 2 hours with either TNFα (10 ng/ml) or PMA (10 ng/ml). Wildtype mouse embryonic fibroblasts were also stimulated for 2 hours with either TNFα (10 ng/ml) or PMA (10 ng/ml) as served as a positive control. Cell lines not subject to stimulation are labelled as non-stimulated (“NS”). As shown, expression of the mouse homologue of AD037 was constitutive in wild type fibroblasts. In contrast, no expression was detected in fibroblasts derived from either p65 or RelB deficient fibroblasts. Reduced levels of AD037 were detected in fibroblasts derived from p50 knockouts. These data suggest that complexes containing p65, RelB, and p50 are required for AD037 expression. Expression data was obtained by measuring the steady state AD037 mRNA levels by quantitative PCR using the PCR primer pair provided as SEQ ID NO:285 and 286 as described herein.

FIG. 82 shows the level of IL-8 expressed induced in response to transfection of H292 epithelial cells with expression constructs encoding either wild type IKK2 or wild type AD037, in the presence or absence of TNFα. As expected, transfection of wild type IKK2 significantly increased both basal and induced levels of IL-8 as compared to transfection with vector alone. Transfection of wild type AD037 also increased both basal and induced levels of IL-8 above that stimulated by vector, or by IKK2. These data suggest that AD037 can functionally interact with the NF-κB pathway. IL-8 expression was determined by measuring the level of IL-8 protein using ELISA as described herein.

FIG. 83 shows a Western blot of COS cells transfected with expression vectors containing either the wild type AD037 coding region (“WT”), the AD037 coding region with the Ras association motif deleted (“Δras”), or the AD037 coding region with the consensus myristoylation site deleted (“Δmyr”). Each construct contained a Flag epitope tag. As shown, each of the three constructs expressed AD037 protein. Blots were probed with a mouse monoclonal IgG specific for the Flag epitope tag (Sigma, St. Louis, Mo.), followed by detection with HRP-conjugated antibodies specific for mouse IgG, and ECL (Amersham Pharmacia Biotech, Piscataway, N.J.), as described herein.

FIG. 84 shows the level of IL-8 expression in H292 epithelial cells transfected with expression vectors containing either the wild type IKK-2 coding region (“IKK-2”), the wild type AD037 coding region (“AD037”), the AD037 coding region with the Ras association motif deleted (“AD037ras”), or the AD037 coding region with the consensus myristoylation site deleted (“AD037myr”) with 0.25 ug or 0.5 ug of vector, and in the presence or absence of TNFα. As shown, expression of wild type IKK-2 and wild type AD037 significantly increased basal and induced levels of IL-8 above that detected in cells transfected with vector alone. Expression of either the myristoylation site deletion or the Ras Association motif AD037 mutant failed to increase IL-8 levels above that detected in the vector controls. This data indicates that both motifs are required for AD037 function. IL-8 expression was determined by measuring the level of IL-8 protein using ELISA as described herein.

FIG. 85 shows the protein structure of the Wild type AD037 polypeptide (SEQ ID NO:126). Boxes indicate positions of the peptide sequence used to generate rabbit antisera specific for AD037 (denoted as “Ab”), a putative myristoylation site (denoted as “Myr”), and a Ras association motif (“denoted as Ras Assoc.”).

FIG. 86 shows a Western blot of whole cell lysates of THP-1 monocytes transfected with an expression vector containing the coding region of the wild-type AD037 polypeptide subsequent to simulation of with LPS (100 ng/ml; denoted as “LPS”) and/or in the presence and absence of BMS-205820 (denoted as “p”) for 4, 8, or 24 hours. Bands were detected with HRP-tagged anti-rabbit antibodies followed by ECL. The arrow indicates a specific band that is blocked when the rabbit antisera is preincubated with immunizing peptide. As shown, the expression of the AD037 polypeptide was specifically upregulated in response to LPS, and downregulated in response to LPS and peptide stimulation. The latter is consistent with earlier results obtained by measuring mRNA levels of AD037 in response to the same conditions. Additional experimental conditions are described herein.

FIG. 87 shows confocal microscopic views of Cos7 cells transfected with pcDNA3.1mychis-AD037 expression vector, the pcDNA3.1mychis-AD037 expression vector containing the AD037Δmyr mutant, and the pcDNA3.1mychis-AD037 expression vector containing the AD037Δras mutant, after incubation with anti-Flag antibodies and FITC-labeled secondary antibodies. Cos7 cells transfected with the pcDNA3 vector served as a negative control. As shown, plasma membrane specific fluorescence was detected in cells transfected with AD037 (panel B), but not in cells transfected with vector alone (panel A), nor in cells transfected with either of the AD037Δmyr mutant, or the AD037Δras mutant. The results suggest AD037 associates with membrane-localized protein(s), and that both the myristolation site as well as the Ras association motif are required for membrane localization.

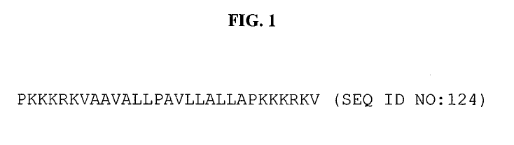

Table I provides a summary of the NFkB associated polynucleotides and polypeptides of the present invention. ‘Clone Name’ refers to the unique identifier provided for each sequence. ‘Genbank Accession No:’ provides the Genbank Accession number of the corresponding genomic sequence for each polynucleotide sequence of the present invention. The ‘Genbank Accession No’ may also represent the name of the unique identifier for each sequence. The other columns are defined elsewhere herein.

Table II provides the polynucleotide and polypeptide sequences of each clone referenced in Table I.

Table III provides a summary of the NFkB associated polynucleotides and polypeptides of the present invention that were identified using microarray methodology as described herein.

Table IV provides the polynucleotide and polypeptide sequences of each clone referenced in Table III.

Table V provides the Genbank Accession No. and/or the Incyte Accession number of the sequences used to extend the polynucleotide sequences of the present invention. The present invention encompasses the use of these sequences for any of the uses described herein for the NFkB associated sequences. The information contained within the following accession numbers in addition to any accession numbers referenced herein, or in the Figures or Tables, is hereby incorporated herein by reference in its entirety.

Table VI provides the hybridization conditions encompassed by the present invention.

Table VII provides the conservative amino acid substitutions encompassed by the present invention.

### DETAILED DESCRIPTION OF THE INVENTION

The present invention may be understood more readily by reference to the following detailed description of the preferred embodiments of the invention and the Examples included herein. The NFkB associated polynucleotides and polypeptides are sometimes refered to herein as “NFkB modulatory” polynucleotides and polypeptides. Likewise, all references to “NFkB associated polynucleotides and polypeptides” shall be construed to apply to “NFkB modulatory polynucleotides and polypeptides”.

The invention provides the polynucleotide and polypeptide sequences of genes that are believed to be associated with the NF-kB pathway. As referenced herein, members of the NFkB family are transcription factors that are critical regulators of inflammatory and stress responses. Thus, the polynucleotide and polypeptides of the present invention may also be represent critical regulators of inflammatory and stress responses.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

As used herein, a “polynucleotide” refers to a molecule having a nucleic acid sequence contained in SEQ ID NO:1-108, 125, 127, 132-140, 158-159, or 264-284. For example, the polynucleotide can contain the nucleotide sequence of the full length cDNA sequence, including the 5′ and 3′ untranslated sequences, the coding region, with or without a signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

In the present invention, the full length sequence identified as SEQ ID NO:1-108, 125, 127, 132-140, 158-159, or 264-284 was often generated by overlapping sequences contained in multiple clones (contig analysis), or extended using known sequences as described herein.

Unless otherwise indicated, all nucleotide sequences determined by sequencing a DNA molecule herein were determined using an automated DNA sequencer (such as the Model 373, preferably a Model 3700, from Applied Biosystems, Inc.), and all amino acid sequences of polypeptides encoded by DNA molecules determined herein were predicted by translation of a DNA sequence determined above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded bt the sequenced DNA molecule, beginning at the point of such an insertion or deletion.

Using the information provided herein, such as the nucleotide sequences provided in the Sequence Listing (SEQ ID NO:1-108, 125, 127, 132-140, 158-159, and 264-284), a nucleic acid molecule of the present invention encoding the polypeptides of the present invention may be obtained using standard cloning and screening procedures, such as those for cloning cDNAs using mRNA as starting material. Illustrative of the invention, the nucleic acid molecules described herein (SEQ ID NO:1-108, 125, 127, 132-140, 158-159, and 264-284) were discovered based upon their differential expression in a human monocyte cell line upon the administration of an NFkB peptide inhibitor.

A “polynucleotide” of the present invention also includes those polynucleotides capable of hybridizing, under stringent hybridization conditions, to sequences contained in SEQ ID NO:1-108, 125, 127, 132-140, 158-159, or 264-284, the complement thereof. “Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65 degree C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37 degree C. in a solution comprising 6×SSPE (20X SSPE=3M NaCl; 0.2M NaH2PO4; 0.02M EDTA, pH 7.4), 0.5% SDS, 30% formamide, 100 ug/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone generated using oligo dT as a primer).

The polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure and Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:1-108, 125, 127, 132-140, 158-159, or 264-284” refers to a polynucleotide sequence while “SEQ ID NO:109-118, 126, 128, 144-152, or 160-161” refers to a polypeptide sequence, both sequences identified by an integer specified in Table 1.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

The term “organism” as referred to herein is meant to encompass any organism referenced herein, though preferably to eukaryotic organisms, more preferably to mammals, and most preferably to humans.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The present invention encompasses the identification of proteins, nucleic acids, or other molecules, that bind to polypeptides and polynucleotides of the present invention (for example, in a receptor-ligand interaction). The polynucleotides of the present invention can also be used in interaction trap assays (such as, for example, that discribed by Ozenberger and Young (Mol Endocrinol., 9(10):1321-9, (1995); and Ann. N. Y. Acad. Sci., 7;766:279-81, (1995)).

The polynucleotide and polypeptides of the present invention are useful as probes for the identification and isolation of full-length cDNAs and/or genomic DNA which correspond to the polynucleotides of the present invention, as probes to hybridize and discover novel, related DNA sequences, as probes for positional cloning of this or a related sequence, as probe to “subtract-out” known sequences in the process of discovering other novel polynucleotides, as probes to quantify gene expression, and as probes for microarays.

In addition, polynucleotides and polypeptides of the present invention may comprise one, two, three, four, five, six, seven, eight, or more membrane domains.

Also, in preferred embodiments the present invention provides methods for further refining the biological function of the polynucleotides and/or polypeptides of the present invention.

Specifically, the invention provides methods for using the polynucleotides and polypeptides of the invention to identify orthologs, homologs, paralogs, variants, and/or allelic variants of the invention. Also provided are methods of using the polynucleotides and polypeptides of the invention to identify the entire coding region of the invention, non-coding regions of the invention, regulatory sequences of the invention, and secreted, mature, pro-, prepro-, forms of the invention (as applicable).

In preferred embodiments, the invention provides methods for identifying the glycosylation sites inherent in the polynucleotides and polypeptides of the invention, and the subsequent alteration, deletion, and/or addition of said sites for a number of desirable characteristics which include, but are not limited to, augmentation of protein folding, inhibition of protein aggregation, regulation of intracellular trafficking to organelles, increasing resistance to proteolysis, modulation of protein antigenicity, and mediation of intercellular adhesion.

In further preferred embodiments, methods are provided for evolving the polynucleotides and polypeptides of the present invention using molecular evolution techniques in an effort to create and identify novel variants with desired structural, functional, and/or physical characteristics.

The present invention further provides for other experimental methods and procedures currently available to derive functional assignments. These procedures include but are not limited to spotting of clones on arrays, micro-array technology, PCR based methods (e.g., quantitative PCR), anti-sense methodology, gene knockout experiments, and other procedures that could use sequence information from clones to build a primer or a hybrid partner.

As used herein the terms “modulate” or “modulates” refer to an increase or decrease in the amount, quality or effect of a particular activity, DNA, RNA, or protein.

### Polynucleotides and Polypeptides of the Present Invention

The polynucleotide and polypeptides of the present invention were identified based upon their differential expression upon the administration of a known NFkB peptide inhibitor (SEQ ID NO:124) as described herein. As a result, polynucleotide and polypeptides of the present invention are expected to share at least some biological activity with NFkB, and more preferably with NFkB modulators, in addition to agonists or antagonists thereof. While the NFkB-associated sequences are likely to comprise representatives from a number of protein families and classes (such as GPCRs, transcription factors, ion channels, proteases, nucleases, secreted proteins, nuclear hormone receptors, etc.), their biological activity will likely not be exactly the same as NFkB (e.g., a transciption factor). Rather the NFkB associated polynucleotides and polypeptides of the present invention are believed to represent either direct, or indirect, participating members of the NFkB pathway. Therefore, it is expected that the NFkB associated polynucleotides and polypeptides of the present invention, including agonists, antagonists, or fragments thereof, will be capable of providing at least some of the therapeutic benefits afforded by modulators of NFkB, and potentially NFkB itself, upon administration to a patient in need of treatment. The present invention also encompasses antagonists or agonists of the polynucleotides and polypeptides, including fragments thereof, and their potential utility in modulating NFkB modulators, and potentially NFkB itself.

Modulating the activity of the NFkB associated genes of the present invention may result in fewer toxicities than the drugs, therapies, or regimens presently known to regulate NF-kappaB itself. Such NF-kappaB inhibitors include the following, non-limiting examples: NFkB decoy oligonucleotide-HVJ liposomes complex (Dainippon); gene therapy-based implantation of the I kappa B gene into donor organs ex vivo (Novartis; EP699977); drugs designed to block IkappaBalpha-directed ubiquitination enzymes resulting in more specific suppression of NF-KB activation (Aventis); declopramide (OXiGENE; CAS® Registry Number: 891-60-1); IPL-550260 (Inflazyme); IPL-512602 (Inflazyme); KP-392 (Kinetek); R-flurbiprofen (Encore Pharmaceuticals; E-7869, MPC-7869; (1,1′-Biphenyl)-4-acetic acid, 2-fluoro-alpha-methyl; CAS® Registry Number: 5104-49-4); drugs disclosed in U.S. Pat. Nos. 5,561,161 and 5,340,565 (OXiGENE); dexlipotam (Asta Medica); RIP-3 Rigel (Rigel; Pharmaprojects No. 6135); tyloxapol Discovery (Discovery Laboratories; SuperVent; 4-(1,1,3,3-Tetramethylbutyl)phenol polymer with formaldehyde andoxirane; CAS® Registry Number: 25301-02-4); IZP-97001 (Inflazyme); IZP-96005 (Inflazyme); IZP-96002 (Inflazyme); sortac (Inflazyme; IPL-400); BXT-51072 (OXIS; 2H-1,2-Benzoselenazine, 3,4-dihydro-4,4-dimethyl-; CAS® Registry Number: 173026-17-0); SP-100030 (Celgene; 2-chloro-N-(3,5-di(trifluoromethyl)phenyl)-4-(trifluoromethyl)pyrimidine-5-carboxamide); IPL-576092 (Inflazyme; Stigmastan-15-one, 22,29-epoxy-3,4,6,7,29-pentahydroxy-, (3alpha,4beta,5alpha, 6alpha,7beta, 14beta,22S); CAS® Registry Number: 137571-30-3; U.S. Pat. No. 6,046,185); P54 (Phytopharm); Interleukin-10 (Schering-Plough;SCH 52000; Tenovil; rIL-10; rhIL-10; CAS Registry Number: 149824-15-7); and antisense oligonucleotides PLGA/PEG microparicles.

The NFkB associated polynucleotides and polypeptides of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

Alternatively, antagonists and/or fragments of the NFkB associated polynucleotides and polypeptides of the present invention have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The NFkB associated polynucleotides and polypeptides of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

Alternatively, antagonists of the NFkB associated polynucleotides and polypeptides of the present invention, including fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The NFkB associated polynucleotides and polypeptides of the present invention are useful in diagnosing individuals susceptible to diseases and disorders associated with aberrant NFkB activity.

To confirm the NF-kB regulation of these genes, monocytes can be stimulated with LPS in the presence and absence of NF-kB inhibitors including dexamethasone, and BMS-205820. RNA can then be isolated from these cells and used in RT-PCR reactions with gene specific primers. RT-PCR reactions can also be performed to determine tissue expression patterns for each gene. The functional relevance of these genes in an NF-kB dependent response can be tested using antisense oligonucleotides. The human monocyte line THP-1 can be electroporated with gene specific antisense oligonucleotides, and then stimulated with LPS to induce TNFα secretion. Antisense oligonucleotides that inhibit or augment TNFα secretion can indicate those genes that are functionally involved in an NF-kB dependent pathway. The inhibition of expression of other known NF-kB target genes such as adhesion molecules, or other cytokines may also be monitored. The results of many of these latter experiments are described herein for the NFkB associated polynucleotides and polypeptides of the present invention.

Many polynucleotide sequences, such as EST sequences, are publicly available and accessible through sequence databases. Some of these sequences are related to SEQ ID NO:1-108, 125, 127, 132-140, 158-159, and 264-284 and may have been publicly available prior to conception of the present invention. Preferably, such related polynucleotides are specifically excluded from the scope of the present invention. To list every related sequence would be cumbersome, although a representative list is provided in Table V herein. Accordingly, preferably excluded from the present invention are one or more polynucleotides consisting of a nucleotide sequence described by the general formula of a−b, where a is any integer corresponding to SEQ ID NO: SEQ ID NO:1-108, 125, 127, 132-140, 158-159, and 264-284, and b is any integer corresponding to SEQ ID NO: SEQ ID NO:1-108, 125, 127, 132-140, 158-159, and 264-284, where both a and b correspond to the positions of nucleotide residues shown in SEQ ID NO:SEQ ID NO:1-108, 125, 127, 132-140, 158-159, and 264-284, and where b is greater than or equal to a+14.

### Features of the Polypeptide Encoded by Gene No:7

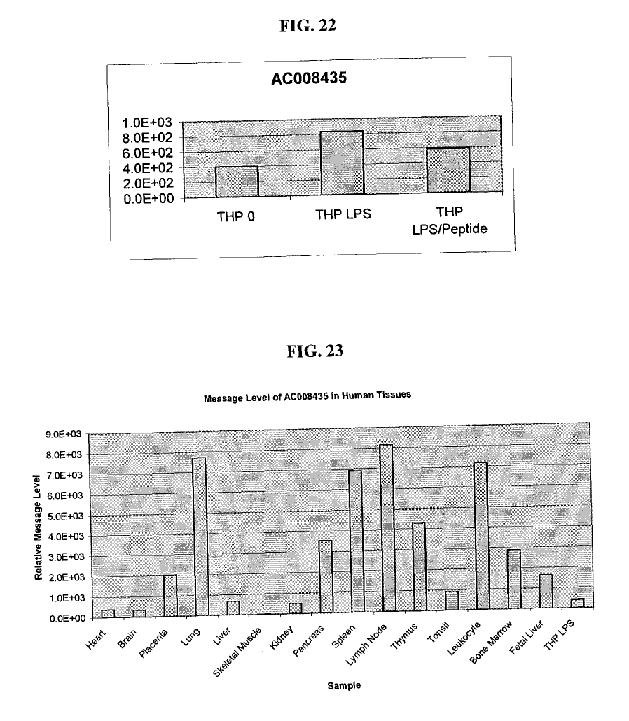

In confirmation that the Ac008435 (SEQ ID NO:7, SEQ ID NO:264; Table II) polynucleotide and/or its encoded polypeptide are involved in the NF-kB pathway, real-time PCR analyses was used to show that Ac008435 expression is NF-kB-dependent, as shown in FIG. 22. Ac008435 was expressed in unstimulated THP-1 monocytes as a control. In response to stimulation with LPS, steady-state levels of Ac008435 mRNA increased. This increase in expression was inhibited by inclusion of the selective NF-kB inhibitor, BMS-205820.

In an effort to identify additional associations of the Ac008435 polynucleotide and/or its encoded polypeptide with the NF-kB pathway in other human tissues, RT-PCR was performed on a variety of tissues. The results of these experiments indicate that Ac008435 mRNA is expressed at predominately high levels in immune and hematopoietic tissues including lymph node, leukocytes, and spleen. High levels of expression were also detected in non-hematopoietic tissues including the lung, and pancreas. Lower levels of expression were detected in thymus, pancreas, bone marrow, fetal liver, and placenta (see FIG. 23). The increased expression levels in immune tissues is consistent with the Ac008435 representing a NFkB modulated polynucleotide and polypeptide.

The confirmation that the expression of the Ac008435 polynucleotide and encoded peptide are inhibited by NFkB suggests that antagonists directed against the Ac008435 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the Ac008435 polynucleotide and/or encoded peptide are useful for decreasing NF-kB activity, decreasing apoptotic events, and/or increasing IkBa expression or activity levels.

The AC008435 NFkB associated polynucleotide and polypeptide of the present invention, including antagonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The AC008435 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The predominate expression in lymph node, leukocytes, spleen, thymus, bone marrow, and fetal liver tissue, in combination with its association with the NFkB pathway suggests the Ac008435 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression of Ac008435 transcripts in lung tissue, in combination with its association with the NFkB pathway suggests the potential utility for Ac008435 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

### Features of the Polypeptide Encoded by Gene No:8

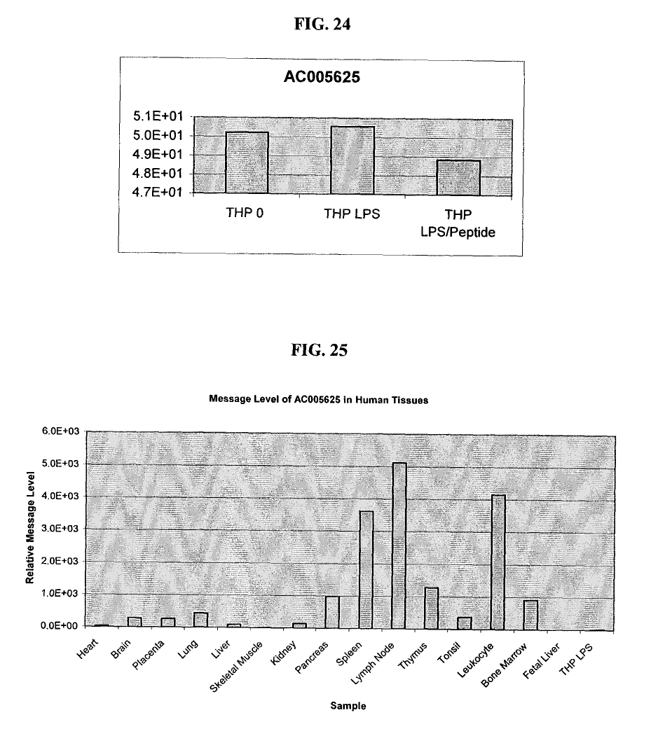

In confirmation that the Ac005625 (SEQ ID NO:8; Table II) polynucleotide and/or its encoded polypeptide are involved in the NF-kB pathway, real-time PCR analyses was used to show that Ac005625 expression is NF-kB-dependent, as shown in FIG. 24. Ac005625 was expressed in unstimulated THP-1 monocytes as a control. In response to stimulation with LPS, steady-state levels of Ac005625 mRNA increased. This increase in expression was inhibited by inclusion of the selective NF-kB inhibitor, BMS-205820.

In an effort to identify additional associations of the Ac005625 polynucleotide and/or its encoded polypeptide with the NF-kB pathway in other human tissues, RT-PCT was performed on a variety of tissues. The results of these experiments indicate that Ac005625 mRNA is expressed at predominately high levels in immune and hematopoietic tissues including lymph node, spleen, leukocytes, and to a lesser extent in thymus and bone marrow. Significant expression was also detected in pancreas, in addition to other tissues as shown (see FIG. 25). The increased expression levels in immune tissues is consistent with the Ac005625 representing a NFkB modulated polynucleotide and polypeptide.

The confirmation that the expression of the Ac005625 polynucleotide and encoded peptide are inhibited by NFkB suggests that antagonists directed against the Ac005625 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the Ac005625 polynucleotide and/or encoded peptide are useful for decreasing NF-kB activity, decreasing apoptotic events, and/or increasing IkBa expression or activity levels.

The AC005625 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AC005625 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The predominate expression in lymph node, spleen, leukocytes, thymus, and bone marrow tissue, in combination with its association with the NFkB pathway suggests the Ac005625 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:9

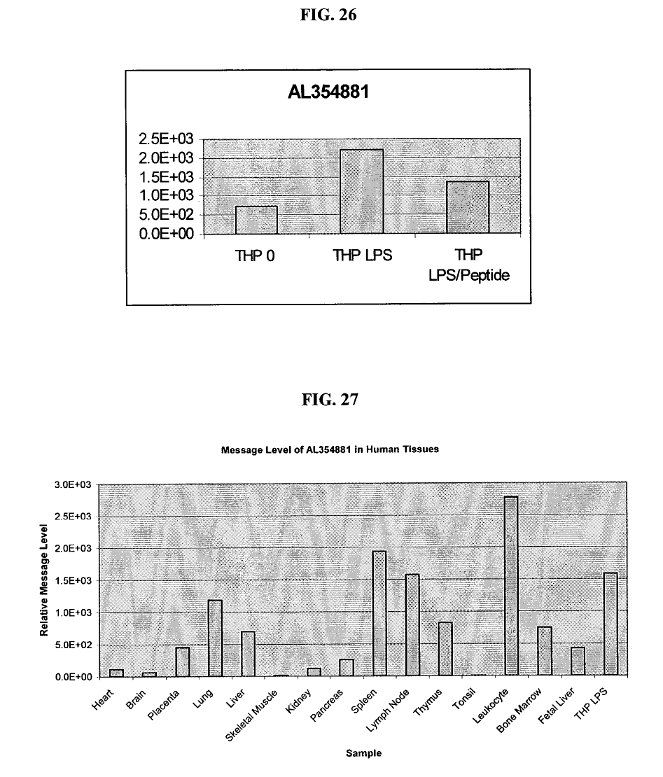

In confirmation that the Ac354881 (SEQ ID NO:9; SEQ ID NO:265; Table II) polynucleotide and/or its encoded polypeptide are involved in the NF-kB pathway, real-time PCR analyses was used to show that Ac354881 expression is NF-kB-dependent, as shown in FIG. 26. Ac354881 was expressed in unstimulated THP-1 monocytes as a control. In response to stimulation with LPS, steady-state levels of Ac354881 mRNA increased. This increase in expression was inhibited by inclusion of the selective NF-kB inhibitor, BMS-205820.

In an effort to identify additional associations of the Ac354881 polynucleotide and/or its encoded polypeptide with the NF-kB pathway in other human tissues, RT-PCR was performed on a variety of tissues. The results of these experiments indicate that Ac354881 mRNA is expressed at predominately high levels in immune and hematopoietic tissues including leukocytes, spleen, lymph node, LPS treated THP cells, and to a lesser extent in thymus, bone marrow, and fetal liver. Significant expression was also detected in lung, placemta.liver, in addition to other tissues as shown (see FIG. 27). The increased expression levels in immune tissues is consistent with the Ac354881 representing a NFkB modulated polynucleotide and polypeptide.

The confirmation that the expression of the Ac354881 polynucleotide and encoded peptide are inhibited by NFkB suggests that antagonists directed against the Ac354881 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the Ac354881 polynucleotide and/or encoded peptide are useful for decreasing NF-kB activity, decreasing apoptotic events, and/or increasing IkBa expression or activity levels.

The AC354881 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AC354881 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The predominate expression in leukocytes, spleen, lymph node, LPS treated THP cells, thymus, bone marrow, and fetal liver tissue, in combination with its association with the NFkB pathway suggests the Ac354881 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No: 10

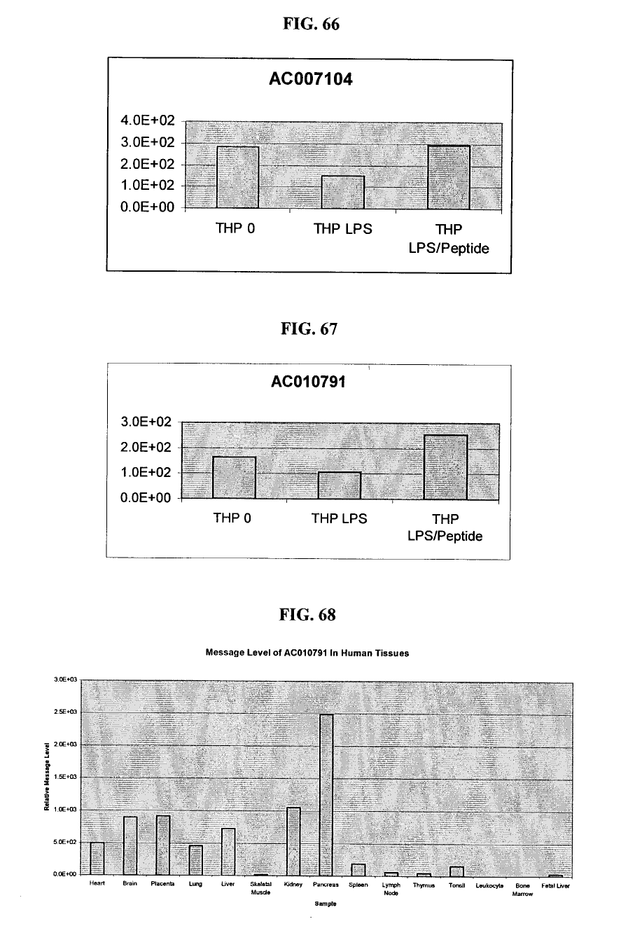

In confirmation that the AC007104 (SEQ ID NO:10; SEQ ID NO:280; Table II) polynucleotide and/or its encoded polypeptide are involved in the NF-kB pathway, real-time PCR analyses was used to show that AC007104 expression is NF-kB-dependent, as shown in FIG. 66. AC007104 was expressed in unstimulated THP-1 monocytes as a control. In response to stimulation with LPS, steady-state levels of AC007104 mRNA increased. This increase in expression was specifically increased by inclusion of the selective NF-kB inhibitor, BMS-205820.

The confirmation that the expression of the AC007104 polynucleotide and encoded peptide are inhibited by NFkB suggests that agonists directed against the AC007104 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the AC007104 polynucleotide and/or encoded peptide are useful for decreasing NF-kB activity, decreasing apoptotic events, and/or increasing IkBa expression or activity levels.

The AC007104 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The AC007104 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

### Features of the Polypeptide Encoded by Gene No:11

In confirmation that the AC010791 (SEQ ID NO:11; SEQ ID NO:281; Table II) polynucleotide and/or its encoded polypeptide are involved in the NF-kB pathway, real-time PCR analyses was used to show that AC010791 expression is NF-kB-dependent, as shown in FIG. 67. AC010791 was expressed in unstimulated THP-1 monocytes as a control. In response to stimulation with LPS, steady-state levels of AC010791 mRNA increased. This increase in expression was specifically increased by inclusion of the selective NF-kB inhibitor, BMS-205820.

In an effort to identify additional associations of the AC010791 polynucleotide and/or its encoded polypeptide with the NF-kB pathway in other human tissues, RT-PCR was performed on a variety of tissues. The results of these experiments indicate that AC010791 mRNA is expressed at predominately high levels in pancreas, and to a lesser extent in kidney, placenta, brain, liver, lung, heart, in addition to other tissues as shown (see FIG. 68).

In further confirmation that the AC010791 is associated with the NFkB pathway, either directly or indirectly, antisense oligonucleotides directed against AC010791 were shown to result in inhibition of E-selectin expression in HMVEC cells stimulated with TNF-alpha according to the assay described in Example 9 herein.

The confirmation that the expression of the AC010791 polynucleotide and encoded peptide are inhibited by NFkB suggests that agonists directed against the AC010791 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the AC010791 polynucleotide and/or encoded peptide are useful for decreasing NF-kB activity, decreasing apoptotic events, and/or increasing IkBa expression or activity levels.

The AC010791 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The AC010791 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The expression in pancreas, in combination with its association with the NFkB pathway suggests the AC010791 polynucleotides and polypeptides, and particularly agonists, may be useful in treating, diagnosing, prognosing, and/or preventing pancreatic, in addition to metabolic and gastrointestinal disorders. In preferred embodiments, 346607 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

### Features of the Polypeptide Encoded by Gene No:14

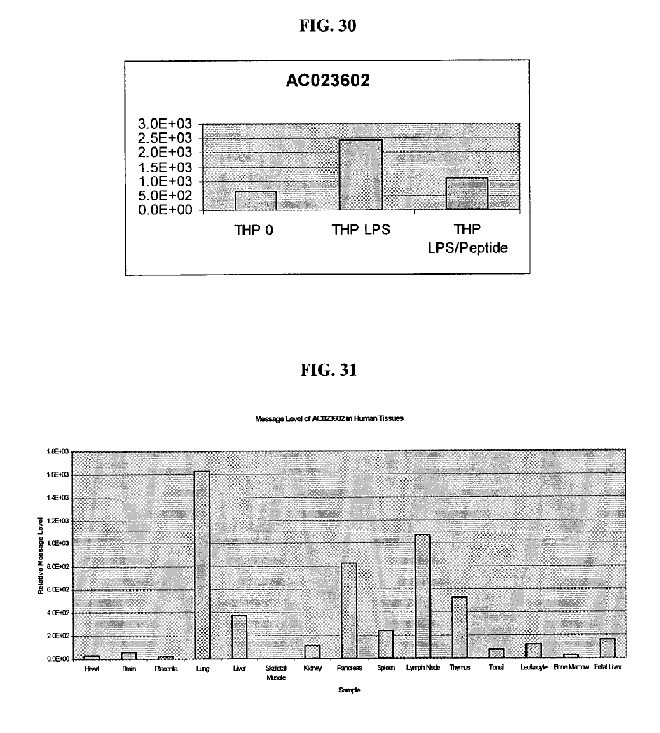

In confirmation that the Ac023602 (SEQ ID NO:14; SEQ ID NO:266; Table II) polynucleotide and/or its encoded polypeptide are involved in the NF-kB pathway, real-time PCR analyses was used to show that Ac023602 expression is NF-kB-dependent, as shown in FIG. 30. Ac023602 was expressed in unstimulated THP-1 monocytes as a control. In response to stimulation with LPS, steady-state levels of Ac023602 mRNA increased. This increase in expression was inhibited by inclusion of the selective NF-kB inhibitor, BMS-205820.

In an effort to identify additional associations of the Ac023602 polynucleotide and/or its encoded polypeptide with the NF-kB pathway in other human tissues, RT-PCR was performed on a variety of tissues. The results of these experiments indicate that Ac023602 mRNA is expressed at predominately high levels in lung, lymph node, pancreas, thymus, and to a lesser extent in liver, spleen, and fetal liver (see FIG. 31). The increased expression levels in immune tissues is consistent with the Ac023602 representing a NFkB modulated polynucleotide and polypeptide.

The confirmation that the expression of the Ac023602 polynucleotide and encoded peptide are inhibited by NFkB suggests that antagonists directed against the Ac023602 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the Ac023602 polynucleotide and/or encoded peptide are useful for decreasing NF-kB activity, decreasing apoptotic events, and/or increasing IkBa expression or activity levels.

The AC023602 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AC023602 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression of Ac023602 transcripts in lung tissue, in combination with its association with the NFkB pathway suggests the potential utility for Ac023602 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

The expression in pancreas tissue, in combination with its association with the NFkB pathway suggests the Ac023602 polynucleotides and polypeptides, preferably antagonists, may be useful in treating, diagnosing, prognosing, and/or preventing pancreatic, in addition to metabolic and gastrointestinal disorders. In preferred embodiments, Ac023602 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

The expression in lymph node, leukocytes, spleen, LPS treated THP cells, thymus, bone marrow, and tonsil tissue, in combination with its association with the NFkB pathway suggests the Ac023602 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:21

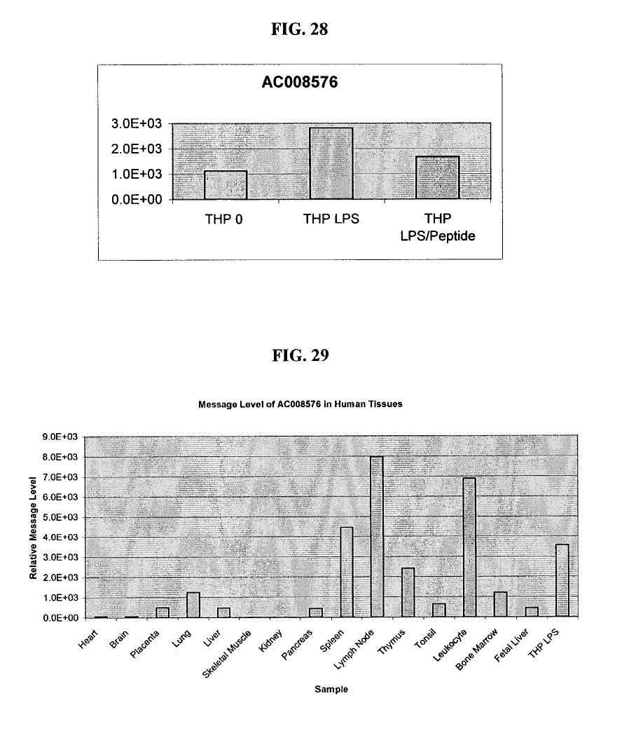

In confirmation that the Ac008576 (SEQ ID NO:21; Table II) polynucleotide and/or its encoded polypeptide are involved in the NF-kB pathway, real-time PCR analyses was used to show that Ac008576 expression is NF-kB-dependent, as shown in FIG. 28. Ac008576 was expressed in unstimulated THP-1 monocytes as a control. In response to stimulation with LPS, steady-state levels of Ac008576 mRNA increased. This increase in expression was inhibited by inclusion of the selective NF-kB inhibitor, BMS-205820.

In an effort to identify additional associations of the Ac008576 polynucleotide and/or its encoded polypeptide with the NF-kB pathway in other human tissues, RT-PCR was performed on a variety of tissues. The results of these experiments indicate that Ac008576 mRNA is expressed at predominately high levels in immune and hematopoietic tissues including lymph node, leukocytes, spleen, LPS treated THP cells, and to a lesser extent in thymus, bone marrow, tonsil, and fetal liver (see FIG. 29). The increased expression levels in immune tissues is consistent with the Ac008576 representing a NFkB modulated polynucleotide and polypeptide.

The confirmation that the expression of the Ac008576 polynucleotide and encoded peptide are inhibited by NFkB suggests that antagonists directed against the Ac008576 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the Ac008576 polynucleotide and/or encoded peptide are useful for decreasing NF-kB activity, decreasing apoptotic events, and/or increasing IkBa expression or activity levels.

The AC008576 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AC008576 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The predominate expression in lymph node, leukocytes, spleen, LPS treated THP cells, thymus, bone marrow, and tonsil tissue, in combination with its association with the NFkB pathway suggests the Ac008576 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:22

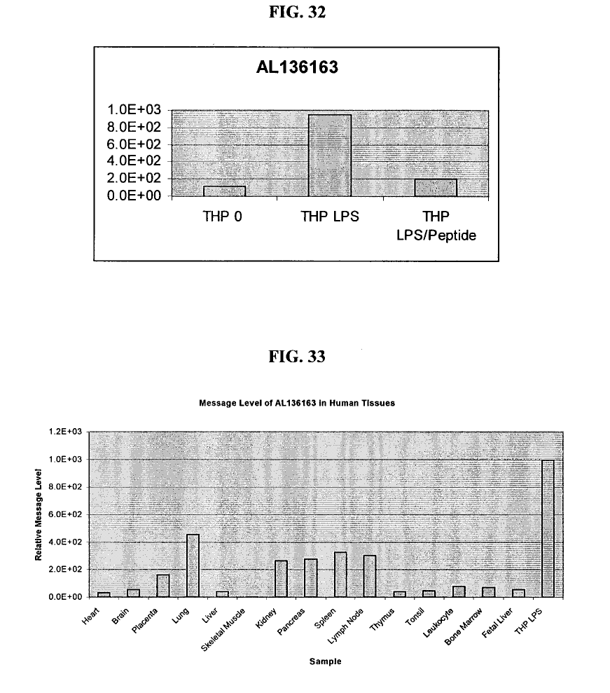

In confirmation that the AL136163 (SEQ ID NO:22; Table II) polynucleotide and/or its encoded polypeptide are involved in the NF-kB pathway, real-time PCR analyses was used to show that AL136163 expression is NF-kB-dependent, as shown in FIG. 32. AL136163 was expressed in unstimulated THP-1 monocytes as a control. In response to stimulation with LPS, steady-state levels of AL136163 mRNA increased. This increase in expression was inhibited by inclusion of the selective NF-kB inhibitor, BMS-205820.

In an effort to identify additional associations of the AL136163 polynucleotide and/or its encoded polypeptide with the NF-kB pathway in other human tissues, RT-PCR was performed on a variety of tissues. The results of these experiments indicate that AL136163 mRNA is expressed at predominately high levels in LPS treated THP cells, and to a lesser extent in lung, spleen, lymph node, pancrease, kidney, in addition to other tissues as shown (see FIG. 33). The increased expression levels in immune tissues is consistent with the AL136163 representing a NFkB modulated polynucleotide and polypeptide.

The confirmation that the expression of the AL136163 polynucleotide and encoded peptide are inhibited by NFkB suggests that antagonists directed against the AL136163 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the AL136163 polynucleotide and/or encoded peptide are useful for decreasing NF-kB activity, decreasing apoptotic events, and/or increasing IkBa expression or activity levels.

The AL136163 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AL136163 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The predominate expression in LPS treated THP cells tissue, in combination with its association with the NFkB pathway suggests the AL136163 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression of AL136163 transcripts in lung tissue, in combination with its association with the NFkB pathway suggests the potential utility for AL136163 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

### Features of the Polypeptide Encoded by Gene No:27

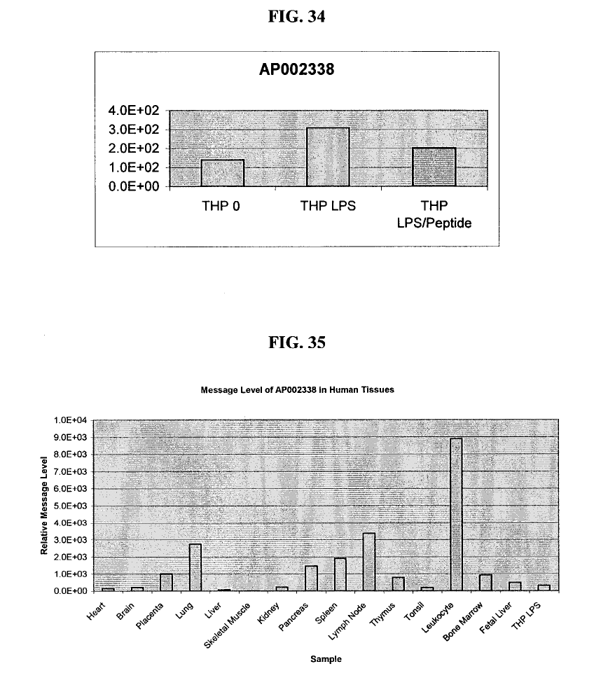

In confirmation that the AP002338 (SEQ ID NO:27; SEQ ID NO:267; Table II) polynucleotide and/or its encoded polypeptide are involved in the NF-kB pathway, real-time PCR analyses was used to show that AP002338 expression is NF-kB-dependent, as shown in FIG. 34. AP002338 was expressed in unstimulated THP-1 monocytes as a control. In response to stimulation with LPS, steady-state levels of AP002338 mRNA increased. This increase in expression was inhibited by inclusion of the selective NF-kB inhibitor, BMS-205820.

In an effort to identify additional associations of the AP002338 polynucleotide and/or its encoded polypeptide with the NF-kB pathway in other human tissues, RT-PCR was performed on a variety of tissues. The results of these experiments indicate that AP002338 mRNA is expressed at predominately high levels in leukocytes, and to a lesser extent in lymph node, lung, spleen, pancrease, in addition to other tissues as shown (see FIG. 35). The increased expression levels in immune tissues is consistent with the AP002338 representing a NFkB modulated polynucleotide and polypeptide.

In further confirmation that the AP002338 is associated with the NFkB pathway, either directly or indirectly, antisense oligonucleotides directed against AP002338 were shown to result in inhibition of E-selectin expression in HMVEC cells stimulated with TNF-alpha according to the assay described in Example 9 herein.

The confirmation that the expression of the AP002338 polynucleotide and encoded peptide are inhibited by NFkB suggests that antagonists directed against the AP002338 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the AP002338 polynucleotide and/or encoded peptide are useful for decreasing NF-kB activity, decreasing apoptotic events, and/or increasing IkBa expression or activity levels.

The AP002338 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AP002338 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The predominate expression in leukocytes and lymph node tissue, in combination with its association with the NFkB pathway suggests the AP002338 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:28

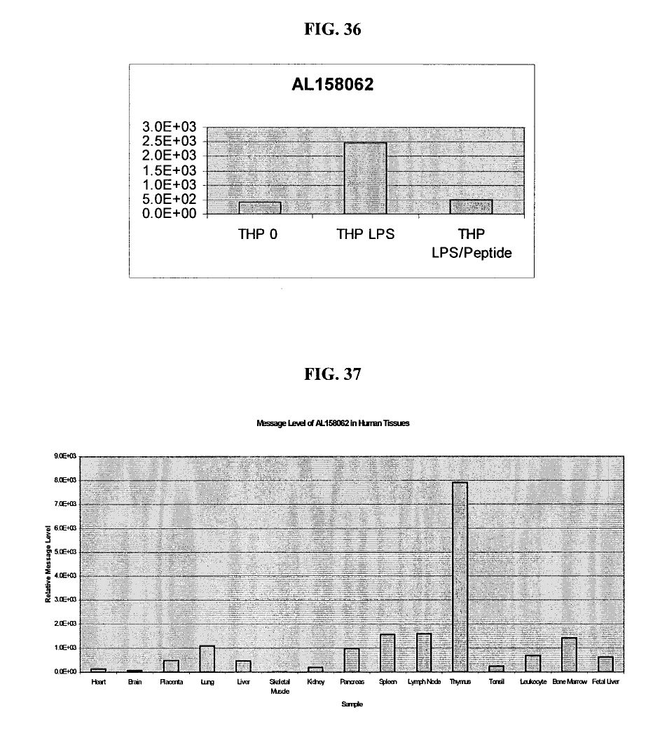

In confirmation that the AL158062 (SEQ ID NO:28; SEQ ID NO:268; Table II) polynucleotide and/or its encoded polypeptide are involved in the NF-kB pathway, real-time PCR analyses was used to show that AL158062 expression is NF-kB-dependent, as shown in FIG. 36. AL158062 was expressed in unstimulated THP-1 monocytes as a control. In response to stimulation with LPS, steady-state levels of AL158062 mRNA increased. This increase in expression was inhibited by inclusion of the selective NF-kB inhibitor, BMS-205820.

In an effort to identify additional associations of the AL158062 polynucleotide and/or its encoded polypeptide with the NF-kB pathway in other human tissues, RT-PCR was performed on a variety of tissues. The results of these experiments indicate that AL158062 mRNA is expressed at predominately high levels in thymus, and to a lesser extent in lymph node, spleen, bone marrow, lung, pancrease, in addition to other tissues as shown (see FIG. 37). The increased expression levels in immune tissues is consistent with the AL158062 representing a NFkB modulated polynucleotide and polypeptide.

The confirmation that the expression of the AL158062 polynucleotide and encoded peptide are inhibited by NFkB suggests that antagonists directed against the AL158062 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the AL158062 polynucleotide and/or encoded peptide are useful for decreasing NF-kB activity, decreasing apoptotic events, and/or increasing IkBa expression or activity levels.

The AL158062 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AL158062 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The predominate expression in thymus tissue, in combination with its association with the NFkB pathway suggests the AL158062 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:33

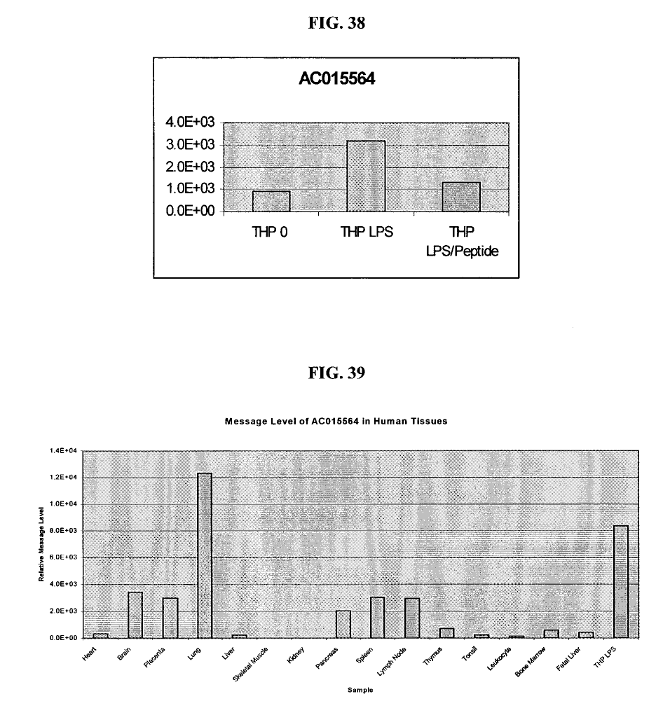

In confirmation that the AC015564 (SEQ ID NO:33; SEQ ID NO:269; Table II) polynucleotide and/or its encoded polypeptide are involved in the NF-kB pathway, real-time PCR analyses was used to show that AC015564 expression is NF-kB-dependent, as shown in FIG. 38. AC015564 was expressed in unstimulated THP-1 monocytes as a control. In response to stimulation with LPS, steady-state levels of AC015564 mRNA increased. This increase in expression was inhibited by inclusion of the selective NF-kB inhibitor, BMS-205820.

In an effort to identify additional associations of the AC015564 polynucleotide and/or its encoded polypeptide with the NF-kB pathway in other human tissues, RT-PCR was performed on a variety of tissues. The results of these experiments indicate that AC015564 mRNA is expressed at predominately high levels in lung, LPS treated THP cells, and to a lesser extent in brain, spleen, lymph node, placenta, pancrease, in addition to other tissues as shown (see FIG. 39). The increased expression levels in immune tissues is consistent with the AC015564 representing a NFkB modulated polynucleotide and polypeptide.

The confirmation that the expression of the AC015564 polynucleotide and encoded peptide are inhibited by NFkB suggests that antagonists directed against the AC015564 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the AC015564 polynucleotide and/or encoded peptide are useful for decreasing NF-kB activity, decreasing apoptotic events, and/or increasing IkBa expression or activity levels.

The AC015564 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AC015564 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression of AC015564 transcripts in lung tissue, in combination with its association with the NFkB pathway suggests the potential utility for AC015564 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

The expression in THP cells, in combination with its association with the NFkB pathway suggests the AC015564 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:36

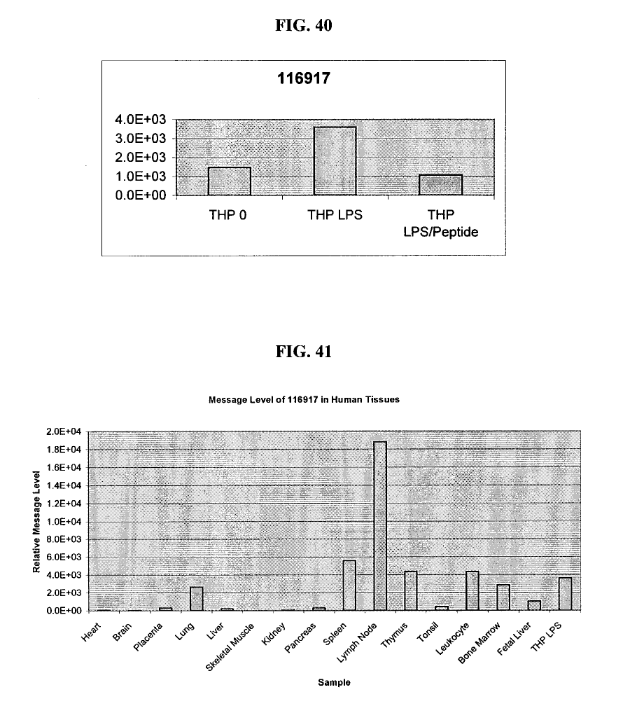

In confirmation that the 116917 (SEQ ID NO:36; SEQ ID NO:270; Table II) polynucleotide and/or its encoded polypeptide are involved in the NF-kB pathway, real-time PCR analyses was used to show that 116917 expression is NF-kB-dependent, as shown in FIG. 40. 116917 was expressed in unstimulated THP-1 monocytes as a control. In response to stimulation with LPS, steady-state levels of 116917 mRNA increased. This increase in expression was inhibited by inclusion of the selective NF-kB inhibitor, BMS-205820.

In an effort to identify additional associations of the 116917 polynucleotide and/or its encoded polypeptide with the NF-kB pathway in other human tissues, RT-PCR was performed on a variety of tissues. The results of these experiments indicate that 116917 mRNA is expressed at predominately high levels in lymph node, and to a lesser extent in, spleen, thymus, leukocyte, LPS treated THP cells, bone marrow, in addition to other tissues as shown (see FIG. 41). The increased expression levels in immune tissues is consistent with the 116917 representing a NFkB modulated polynucleotide and polypeptide.

The confirmation that the expression of the 116917 polynucleotide and encoded peptide are inhibited by NFkB suggests that antagonists directed against the 116917 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 116917 polynucleotide and/or encoded peptide are useful for decreasing NF-kB activity, decreasing apoptotic events, and/or increasing IkBa expression or activity levels.

The 116917 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 116917 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in lymph node, spleen, thymus, leukocyte, LPS treated THP cells, and bone marrow tissue, in combination with its association with the NFkB pathway suggests the 116917 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scieroderma, and modulating cytokine production, antigen presentation, or other processes, such as for boosting immune responses.

Features of the Polypeptide Encoded by Gene No:39

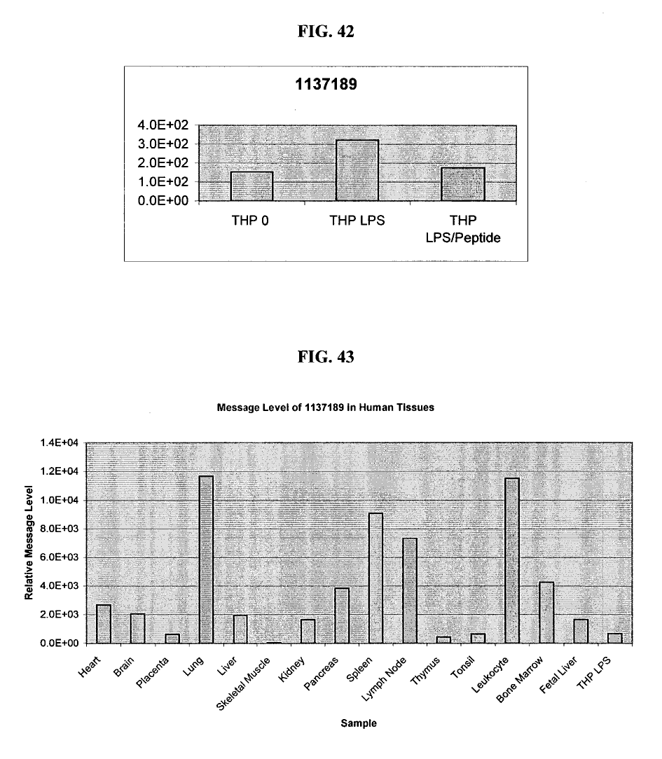

In confirmation that the 1137189 (SEQ ID NO:39; SEQ ID NO:271; Table II) polynucleotide and/or its encoded polypeptide are involved in the NF-kB pathway, real-time PCR analyses was used to show that 1137189 expression is NF-kB-dependent, as shown in FIG. 42. 1137189 was expressed in unstimulated THP-1 monocytes as a control. In response to stimulation with LPS, steady-state levels of 1137189 mRNA increased. This increase in expression was inhibited by inclusion of the selective NF-kB inhibitor, BMS-205820.

In an effort to identify additional associations of the 1137189 polynucleotide and/or its encoded polypeptide with the NF-kB pathway in other human tissues, RT-PCR was performed on a variety of tissues. The results of these experiments indicate that 1137189 mRNA is expressed at predominately high levels in leukocyte, lung, spleen, lymph node, and to a lesser extent in, bone marrow, pancreas, heart, in addition to other tissues as shown (see FIG. 43). The increased expression levels in immune tissues is consistent with the 1137189 representing a NFkB modulated polynucleotide and polypeptide.

The confirmation that the expression of the 1137189 polynucleotide and encoded peptide are inhibited by NFkB suggests that antagonists directed against the 1137189 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 1137189 polynucleotide and/or encoded peptide are useful for decreasing NF-kB activity, decreasing apoptotic events, and/or increasing IkBa expression or activity levels.

The 1137189 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 1137189 NFkB associated polynucleotide and polypeptide of the present invention, including antagonists and/or fragments thereof, have uses that include modulating the phosphorylation of IkB, modulate the activity of IKK-l, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in leukocyte, spleen, lymph node, and bone marrow tissue, in combination with its association with the NFkB pathway suggests the 1137189 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression of 1137189 transcripts in lung tissue, in combination with its association with the NFkB pathway suggests the potential utility for 1137189 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

### Features of the Polypeptide Encoded by Gene No:40

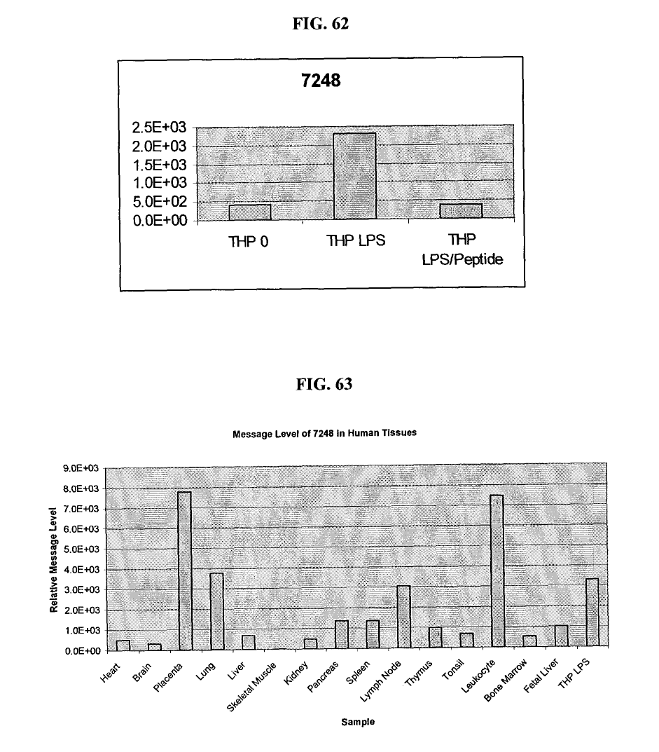

In confirmation that the 7248 (SEQ ID NO:40; SEQ ID NO:279; Table II) polynucleotide and/or its encoded polypeptide are involved in the NF-kB pathway, real-time PCR analyses was used to show that 7248 expression is NF-kB-dependent, as shown in FIG. 62. 7248 was expressed in unstimulated THP-1 monocytes as a control. In response to stimulation with LPS, steady-state levels of 7248 mRNA increased. This increase in expression was inhibited by inclusion of the selective NF-kB inhibitor, BMS-205820.

In an effort to identify additional associations of the 7248 polynucleotide and/or its encoded polypeptide with the NF-kB pathway in other human tissues, RT-PCR was performed on a variety of tissues. The results of these experiments indicate that 7248 mRNA is expressed at predominately high levels in placenta, leukocyte, and to a lesser extent lung, LPS treated THP cells, lymph node, in addition to other tissues as shown (see FIG. 63). The increased expression levels in immune tissues is consistent with the 7248 representing a NFkB modulated polynucleotide and polypeptide.

The confirmation that the expression of the 7248 polynucleotide and encoded peptide are inhibited by NFkB suggests that antagonists directed against the 7248 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 7248 polynucleotide and/or encoded peptide are useful for decreasing NF-kB activity, decreasing apoptotic events, and/or increasing IkBa expression or activity levels.

The 7248 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 7248 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in placenta, in combination with its association with the NFkB pathway suggests the 7248 polynucleotides and polypeptides, preferably antagonists, may be useful in treating, diagnosing, prognosing, and/or preventing reproductive and vascular diseases and/or disorders.

The expression in leukocytes, in combination with its association with the NFkB pathway suggests the 7248 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:46

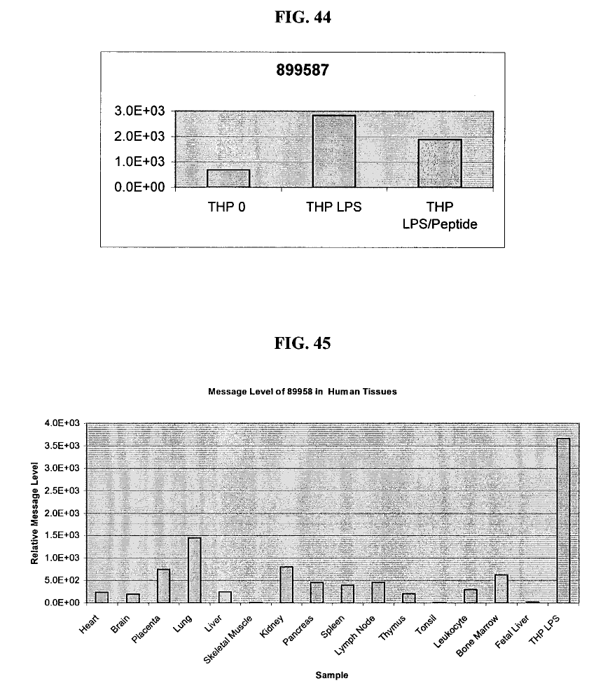

In confirmation that the 899587 (SEQ ID NO:46; SEQ ID NO:272; Table II) polynucleotide and/or its encoded polypeptide are involved in the NF-kB pathway, real-time PCR analyses was used to show that 899587 expression is NF-kB-dependent, as shown in FIG. 44. 899587 was expressed in unstimulated THP-1 monocytes as a control. In response to stimulation with LPS, steady-state levels of 899587 mRNA increased. This increase in expression was inhibited by inclusion of the selective NF-kB inhibitor, BMS-205820.

In an effort to identify additional associations of the 899587 polynucleotide and/or its encoded polypeptide with the NF-kB pathway in other human tissues, RT-PCR was performed on a variety of tissues. The results of these experiments indicate that 899587 mRNA is expressed at predominately high levels in LPS treated THP cells, and to a lesser extent in, lung, placenta, kidney in addition to other tissues as shown (see FIG. 45). The increased expression levels in immune tissues is consistent with the 899587 representing a NFkB modulated polynucleotide and polypeptide.

The confirmation that the expression of the 899587 polynucleotide and encoded peptide are inhibited by NFkB suggests that antagonists directed against the 899587 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 899587 polynucleotide and/or encoded peptide are useful for decreasing NF-kB activity, decreasing apoptotic events, and/or increasing IkBa expression or activity levels.

The 899587 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 899587 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in LPS treated THP cells, in combination with its association with the NFkB pathway suggests the 899587 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression of 899587 transcripts in lung tissue, in combination with its association with the NFkB pathway suggests the potential utility for 899587 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

### Features of the Polypeptide Encoded by Gene No:50

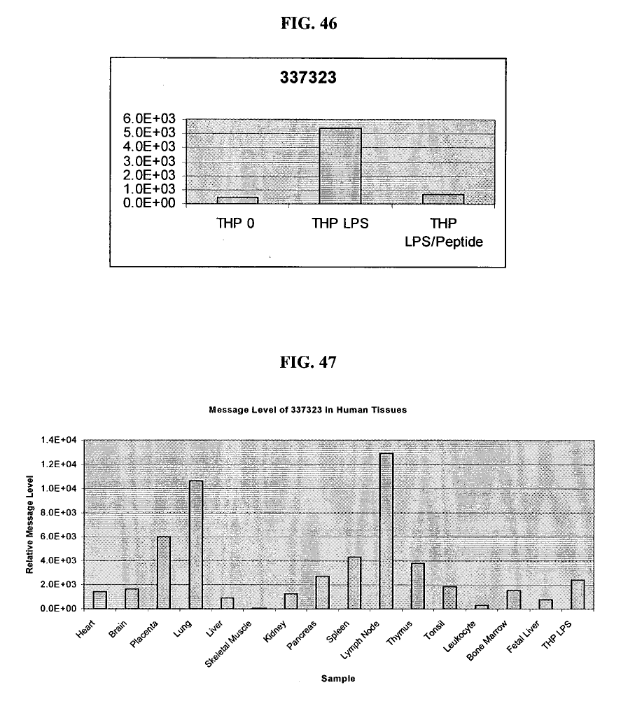

In confirmation that the 337323 (SEQ ID NO:50; SEQ ID NO:273; Table II) polynucleotide and/or its encoded polypeptide are involved in the NF-kB pathway, real-time PCR analyses was used to show that 337323 expression is NF-kB-dependent, as shown in FIG. 46. 337323 was expressed in unstimulated THP-1 monocytes as a control. In response to stimulation with LPS, steady-state levels of 337323 mRNA increased. This increase in expression was inhibited by inclusion of the selective NF-kB inhibitor, BMS-205820.

In an effort to identify additional associations of the 337323 polynucleotide and/or its encoded polypeptide with the NF-kB pathway in other human tissues, RT-PCR was performed on a variety of tissues. The results of these experiments indicate that 337323 mRNA is expressed at predominately high levels in lymph node, lung, and to a lesser extent in, placenta, spleen, thymus, in addition to other tissues as shown (see FIG. 47). The increased expression levels in immune tissues is consistent with the 337323 representing a NFkB modulated polynucleotide and polypeptide.

The confirmation that the expression of the 337323 polynucleotide and encoded peptide are inhibited by NFkB suggests that antagonists directed against the 337323 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 337323 polynucleotide and/or encoded peptide are useful for decreasing NF-kB activity, decreasing apoptotic events, and/or increasing IkBa expression or activity levels.

The 337323 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 337323 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in lymph node, in combination with its association with the NFkB pathway suggests the 337323 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression of 337323 transcripts in lung tissue, in combination with its association with the NFkB pathway suggests the potential utility for 337323 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

### Features of the Polypeptide Encoded by Gene No:52

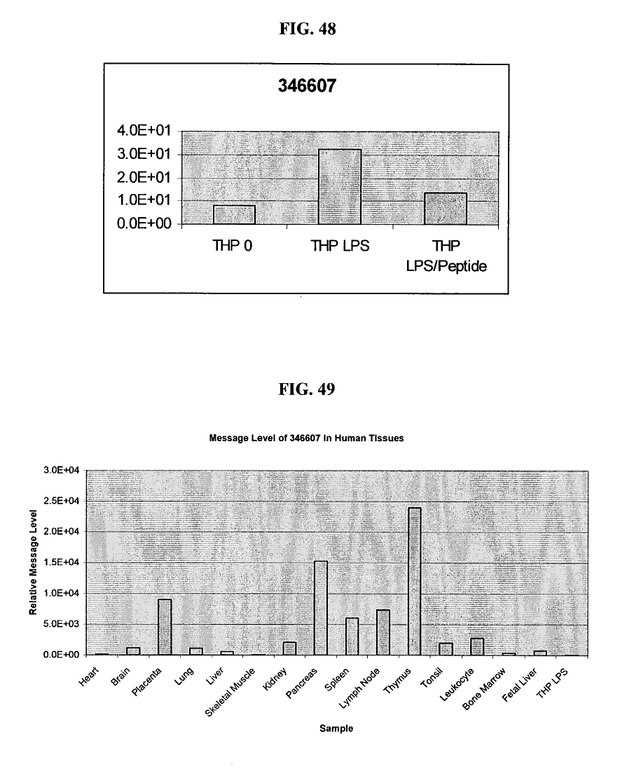

In confirmation that the 346607 (SEQ ID NO:52; SEQ ID NO:274; Table II) polynucleotide and/or its encoded polypeptide are involved in the NF-kB pathway, real-time PCR analyses was used to show that 346607 expression is NF-kB-dependent, as shown in FIG. 48. 346607 was expressed in unstimulated THP-1 monocytes as a control. In response to stimulation with LPS, steady-state levels of 346607 mRNA increased. This increase in expression was inhibited by inclusion of the selective NF-kB inhibitor, BMS-205820.

In an effort to identify additional associations of the 346607 polynucleotide and/or its encoded polypeptide with the NF-kB pathway in other human tissues, RT-PCR was performed on a variety of tissues. The results of these experiments indicate that 346607 mRNA is expressed at predominately high levels in thymus, pancreas, and to a lesser extent in, lung, lymph node, spleen, in addition to other tissues as shown (see FIG. 49). The increased expression levels in immune tissues is consistent with the 346607 representing a NFkB modulated polynucleotide and polypeptide.

The confirmation that the expression of the 346607 polynucleotide and encoded peptide are inhibited by NFkB suggests that antagonists directed against the 346607 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 346607 polynucleotide and/or encoded peptide are useful for decreasing NF-kB activity, decreasing apoptotic events, and/or increasing IkBa expression or activity levels.

The 346607 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 346607 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in thymus, in combination with its association with the NFkB pathway suggests the 346607 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoictic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression in pancreas, in combination with its association with the NFkB pathway suggests the 346607 polynucleotides and polypeptides, preferably antagonists, may be useful in treating, diagnosing, prognosing, and/or preventing pancreatic, in addition to metabolic and gastrointestinal disorders. In preferred embodiments, 346607 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

### Features of the Polypeptide Encoded by Gene No:56

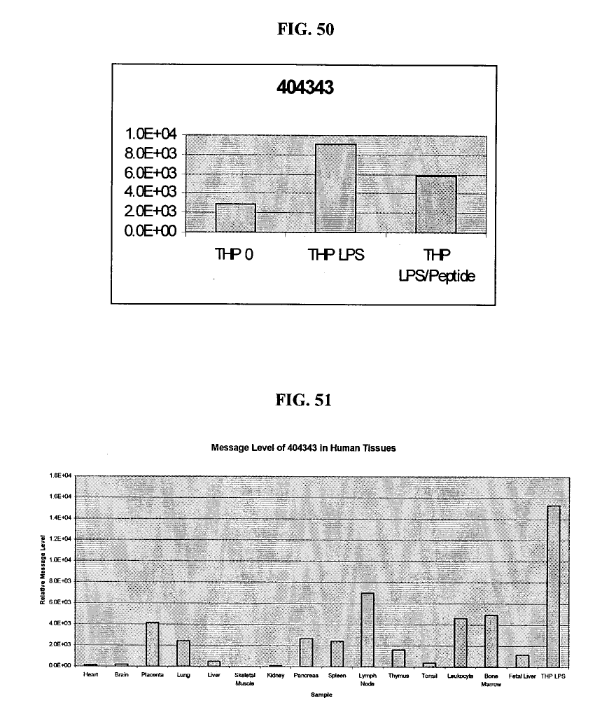

In confirmation that the 404343 (SEQ ID NO:56; SEQ ID NO:275; Table II) polynucleotide and/or its encoded polypeptide are involved in the NF-kB pathway, real-time PCR analyses was used to show that 404343 expression is NF-kB-dependent, as shown in FIG. 50. 404343 was expressed in unstimulated THP-1 monocytes as a control. In response to stimulation with LPS, steady-state levels of 404343 mRNA increased. This increase in expression was inhibited by inclusion of the selective NF-kB inhibitor, BMS-205820.

In an effort to identify additional associations of the 404343 polynucleotide and/or its encoded polypeptide with the NF-kB pathway in other human tissues, RT-PCR was performed on a variety of tissues. The results of these experiments indicate that 404343 mRNA is expressed at predominately high levels in LPS treated THP cells, and to a lesser extent in, lymph node, bone marrow, leukocyte, placenta, in addition to other tissues as shown (see FIG. 51). The increased expression levels in immune tissues is consistent with the 404343 representing a NFkB modulated polynucleotide and polypeptide.

The confirmation that the expression of the 404343 polynucleotide and encoded peptide are inhibited by NFkB suggests that antagonists directed against the 404343 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 404343 polynucleotide and/or encoded peptide are useful for decreasing NF-kB activity, decreasing apoptotic events, and/or increasing IkBa expression or activity levels.

The 404343 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 404343 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in LPS treated THP cells, in combination with its association with the NFkB pathway suggests the 404343 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:57

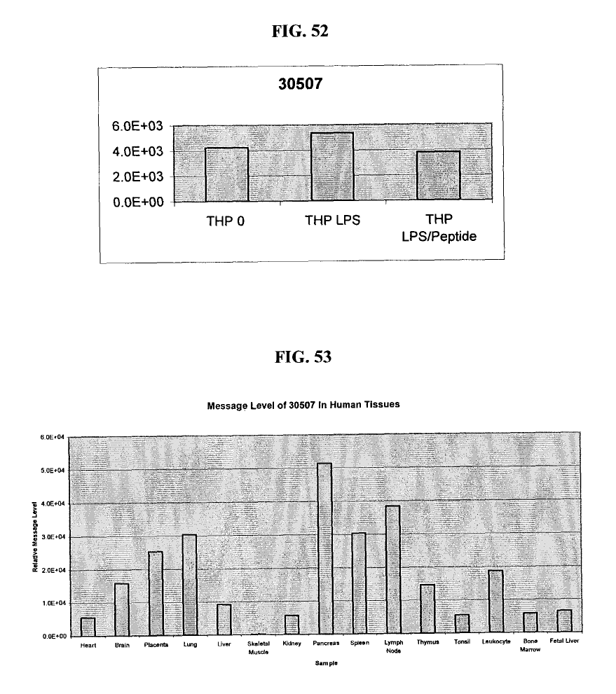

In confirmation that the 30507 (SEQ ID NO:57; SEQ ID NO:276; Table II) polynucleotide and/or its encoded polypeptide are involved in the NF-kB pathway, real-time PCR analyses was used to show that 30507 expression is NF-kB-dependent, as shown in FIG. 52. 30507 was expressed in unstimulated THP-1 monocytes as a control. In response to stimulation with LPS, steady-state levels of 30507 mRNA increased. This increase in expression was inhibited by inclusion of the selective NF-kB inhibitor, BMS-205820.

In an effort to identify additional associations of the 30507 polynucleotide and/or its encoded polypeptide with the NF-kB pathway in other human tissues, RT-PCR was performed on a variety of tissues. The results of these experiments indicate that 30507 mRNA is expressed at predominately high levels in pancreas, lymph node, and to a lesser extent in, spleen, lung, placenta, leukocyte, brain, in addition to other tissues as shown (see FIG. 53). The increased expression levels in immune tissues is consistent with the 30507 representing a NFkB modulated polynucleotide and polypeptide.

In further confirmation that the 30507 is associated with the NFkB pathway, either directly or indirectly, antisense oligonucleotides directed against 30507 were shown to result in inhibition of E-selectin expression in HMVEC cells stimulated with TNF-alpha according to the assay described in Example 9 herein.

The confirmation that the expression of the 30507 polynucleotide and encoded peptide are inhibited by NFkB suggests that antagonists directed against the 30507 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 30507 polynucleotide and/or encoded peptide are useful for decreasing NF-kB activity, decreasing apoptotic events, and/or increasing IkBa expression or activity levels.

The 30507 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 30507 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in lymph node cells, in combination with its association with the NFkB pathway suggests the 30507 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression in pancreas cells, in combination with its association with the NFkB pathway suggests the 30507 polynucleotides and polypeptides, preferably antagonists, may be useful in treating, diagnosing, prognosing, and/or preventing pancreatic, in addition to metabolic and gastrointestinal disorders. In preferred embodiments, 30507 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

### Features of the Polypeptide Encoded by Gene No:62

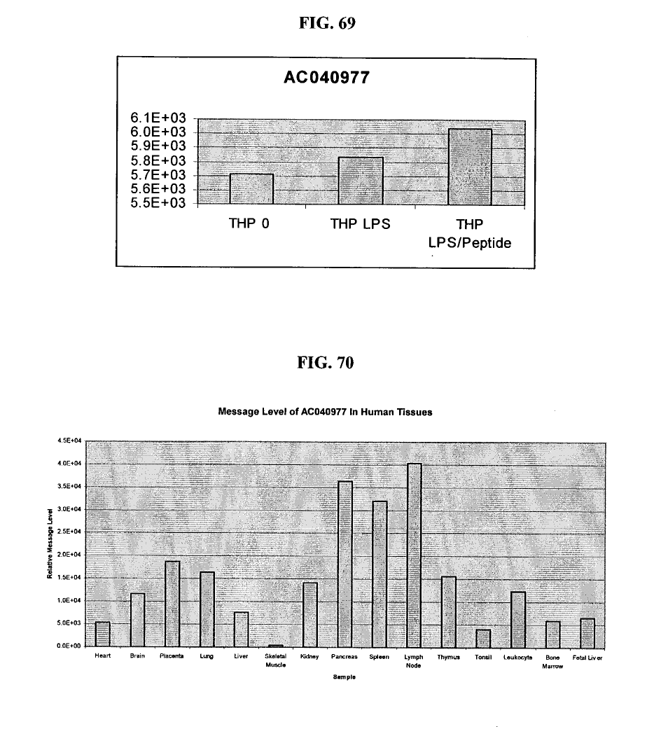

In confirmation that the Ac040977 (SEQ ID NO:62; Table II) polynucleotide and/or its encoded polypeptide are involved in the NF-kB pathway, real-time PCR analyses was used to show that Ac040977 expression is NF-kB-dependent, as shown in FIG. 69. Ac040977 was expressed in unstimulated THP-1 monocytes as a control. In response to stimulation with LPS, steady-state levels of Ac040977 mRNA increased. This increase in expression was specifically increased by inclusion of the selective NF-kB inhibitor, BMS-205820.

In an effort to identify additional associations of the Ac040977 polynucleotide and/or its encoded polypeptide with the NF-kB pathway in other human tissues, RT-PCR was performed on a variety of tissues. The results of these experiments indicate that Ac040977 mRNA is expressed at predominately high levels in lymph node, pancreas, spleen, and to a lesser extent in, placenta, lung, thymus, brain, leukocyte, in addition to other tissues as shown (see FIG. 70). The increased expression levels in immune tissues is consistent with the Ac040977 representing a NFkB modulated polynucleotide and polypeptide.

The confirmation that the expression of the Ac040977 polynucleotide and encoded peptide are inhibited by NFkB suggests that agonists directed against the Ac040977 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the Ac040977 polynucleotide and/or encoded peptide are useful for decreasing NF-kB activity, decreasing apoptotic events, and/or increasing IkBa expression or activity levels.

The AC040977 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The AC040977 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The expression in lymph node and spleen tissue, in combination with its association with the NFkB pathway suggests the Ac040977 polynucleotides and polypeptides, and particularly agonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression in pancreas cells, in combination with its association with the NFkB pathway suggests the Ac040977 polynucleotides and polypeptides, and particularly agonists, may be useful in treating, diagnosing, prognosing, and/or preventing pancreatic, in addition to metabolic and gastrointestinal disorders. In preferred embodiments, Ac040977 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man”syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

### Features of the Polypeptide Encoded by Gene No:67

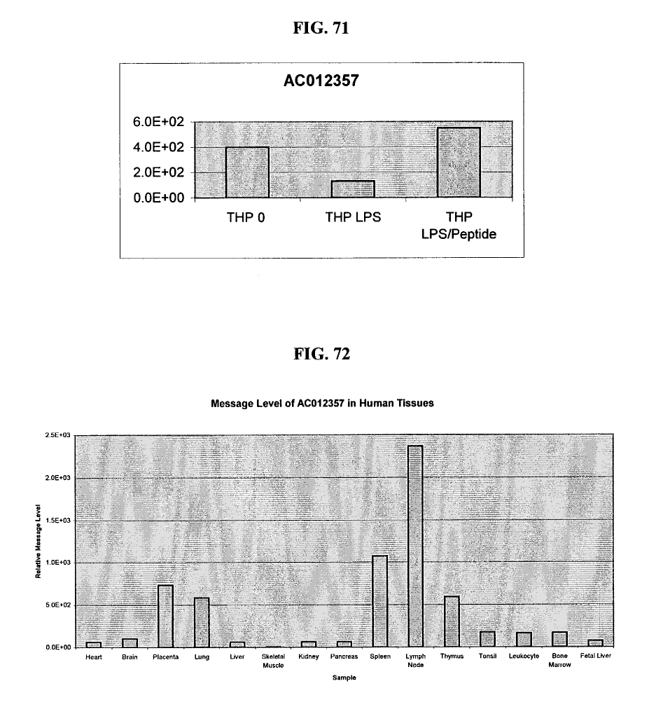

In confirmation that the Ac012357 (SEQ ID NO:67; Table II) polynucleotide and/or its encoded polypeptide are involved in the NF-kB pathway, real-time PCR analyses was used to show that Ac012357 expression is NF-kB-dependent, as shown in FIG. 71. Ac012357 was expressed in unstimulated THP-1 monocytes as a control. In response to stimulation with LPS, steady-state levels of Ac012357 mRNA increased. This increase in expression was specifically increased by inclusion of the selective NF-kB inhibitor, BMS-205820.

In an effort to identify additional associations of the Ac012357 polynucleotide and/or its encoded polypeptide with the NF-kB pathway in other human tissues, RT-PCR was performed on a variety of tissues. The results of these experiments indicate that Ac012357 mRNA is expressed at predominately high levels in lymph node, and to a lesser extent in, spleen, thymus, placenta, in addition to other tissues as shown (see FIG. 72). The increased expression levels in immune tissues is consistent with the Ac012357 representing a NFkB modulated polynucleotide and polypeptide.

The confirmation that the expression of the Ac012357 polynucleotide and encoded peptide are inhibited by NFkB suggests that agonists directed against the Ac012357 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the Ac012357 polynucleotide and/or encoded peptide are useful for decreasing NF-kB activity, decreasing apoptotic events, and/or increasing IkBa expression or activity levels.

The AC012357 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The AC012357 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The expression in lymph node and spleen tissue, in combination with its association with the NFkB pathway suggests the Ac0l2357 polynucleotides and polypeptides, and particularly agonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:70

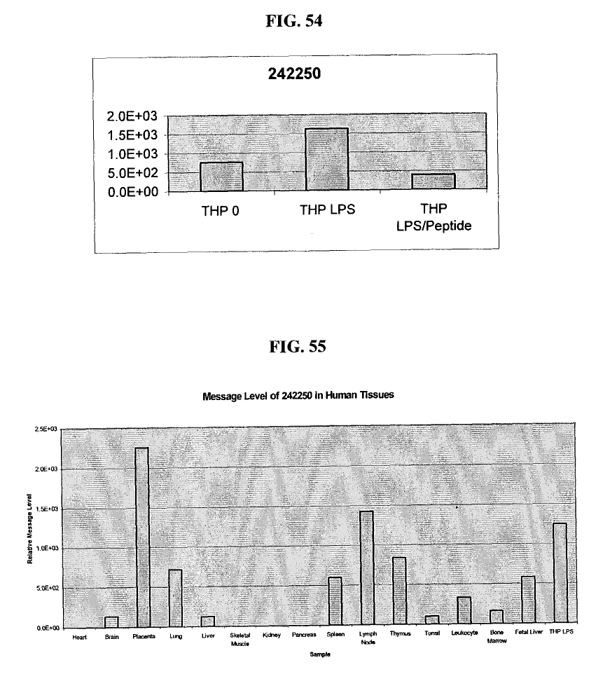

In confirmation that the 242250 (SEQ ID NO:70; SEQ ID NO:277; Table II) polynucleotide and/or its encoded polypeptide are involved in the NF-kB pathway, real-time PCR analyses was used to show that 242250 expression is NF-kB-dependent, as shown in FIG. 54. 242250 was expressed in unstimulated THP-1 monocytes as a control. In response to stimulation with LPS, steady-state levels of 242250 mRNA increased. This increase in expression was inhibited by inclusion of the selective NF-kB inhibitor, BMS-205820.

In an effort to identify additional associations of the 242250 polynucleotide and/or its encoded polypeptide with the NF-kB pathway in other human tissues, RT-PCR was performed on a variety of tissues. The results of these experiments indicate that 242250 mRNA is expressed at predominately high levels in placenta, lymph node, LPS treated THP cells, and to a lesser extent in, thymus, spleen, lung, fetal liver, in addition to other tissues as shown (see FIG. 55). The increased expression levels in immune tissues is consistent with the 242250 representing a NFkB modulated polynucleotide and polypeptide.

The confirmation that the expression of the 242250 polynucleotide and encoded peptide are inhibited by NFkB suggests that antagonists directed against the 242250 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 242250 polynucleotide and/or encoded peptide are useful for decreasing NF-kB activity, decreasing apoptotic events, and/or increasing IkBa expression or activity levels.

The 242250 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 242250 NFkB associated polynucleotide and polypeptide of the present invention, including antagonists and/or fragments thereof, have uses that include modulating the phosphorylation of IkB, modulate the activity of IKK- I, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in placenta, in combination with its association with the NFkB pathway suggests the 242250 polynucleotides and polypeptides, preferably antagonists, may be useful in treating, diagnosing, prognosing, and/or preventing reproductive and vascular diseases and/or disorders.

The expression in lymph node, LPS treated THP cells, in combination with its association with the NFkB pathway suggests the 242250 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:74

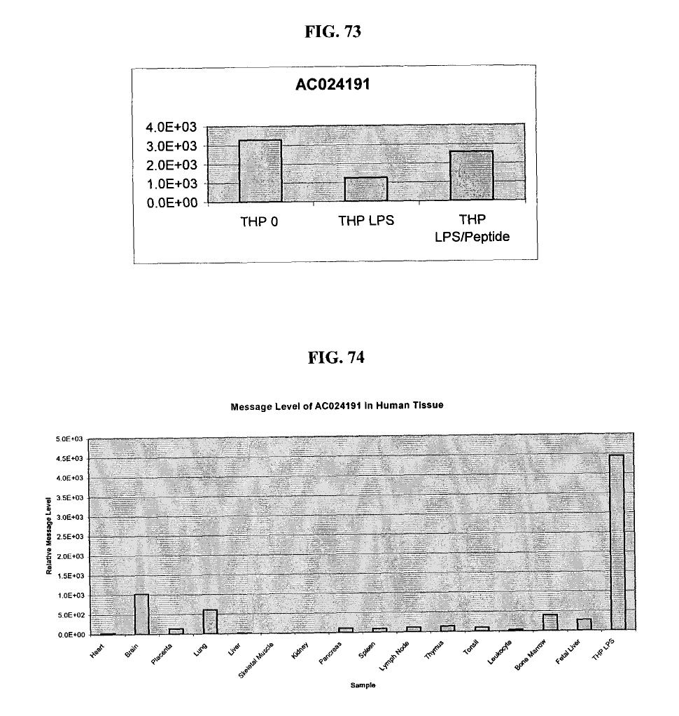

In confirmation that the AC024191 (SEQ ID NO:74; SEQ ID NO:284; Table II) polynucleotide and/or its encoded polypeptide are involved in the NF-kB pathway, real-time PCR analyses was used to show that AC024191 expression is NF-kB-dependent, as shown in FIG. 73. AC024191 was expressed in unstimulated THP-1 monocytes as a control. In response to stimulation with LPS, steady-state levels of AC024191 mRNA increased. This increase in expression was specifically increased by inclusion of the selective NF-kB inhibitor, BMS-205820.

In an effort to identify additional associations of the AC024191 polynucleotide and/or its encoded polypeptide with the NF-kB pathway in other human tissues, RT-PCR was performed on a variety of tissues. The results of these experiments indicate that AC024191 mRNA is expressed at predominately high levels in LPS treated THP cells, and to a lesser extent in other tissues as shown (see FIG. 74). The increased expression levels in immune tissues is consistent with the AC024191 representing a NFkB modulated polynucleotide and polypeptide.

The confirmation that the expression of the AC024191 polynucleotide and encoded peptide are inhibited by NFkB suggests that agonists directed against the AC024191 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the AC024191 polynucleotide and/or encoded peptide are useful for decreasing NF-kB activity, decreasing apoptotic events, and/or increasing IkBa expression or activity levels.

The AC024191 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The AC024191 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The expression in LPS treated THP cells, in combination with its association with the NFkB pathway suggests the AC024191 polynucleotides and polypeptides, and particularly agonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

In preferred embodiments, the following N-terminal AC024191 deletion polypeptides are encompassed by the present invention: M1-L490, D2-L490, G3-L490, N4-L490, D5-L490, N6-L490, V7-L490, T8-L490, L9-L490, L10-L490, F11-L490, A12-L490, P13-L490, L14-L490, L15-L490, R16-L490, D17-L490, N18-L490, Y19-L490, T20-L490, L21-L490, A22-L490, P23-L490, N24-L490, A25-L490, S26-L490, S27-L490, L28-L490, G29-L490, P30-L490, G31-L490, T32-L490, N33-L490, L34-L490, A35-L490, L36-L490, A37-L490, P38-L490, A39-L490, S40-L490, S41-L490, A42-L490, G43-L490, P44-L490, A45-L490, L46-L490, G47-L490, S48-L490, A49-L490, S50-L490, G51-L490, R52-L490, Y53-L490, R54-L490, A55-L490, S56-L490, A57-L490, S58-L490, A59-L490, R60-L490, P61-L490, H62-L490, S63-L490, D64-L490, P65-L490, G66-L490, A67-L490, H68-L490, D69-L490, Q70-L490, R71-L490, P72-L490, R73-L490, G74-L490, R75-L490, R76-L490, G77-L490, E78-L490, P79-L490, R80-L490, P81-L490, F82-L490, P83-L490, V84-L490, P85-L490, S86-L490, A87-L490, L88-L490, G89-L490, A90-L490, P91-L490, R92-L490, A93-L490, P94-L490, V95-L490, L96-L490, G97-L490, H98-L490, A99-L490, A100-L490, E101-L490, P102-L490, R103-L490, A104-L490, E105-L490, R106-L490, V107-L490, R108-L490, G109-L490, R110-L490, R111-L490, L112-L490, C113-L490, I114-L490, T115-L490, M116-L490, L117-L490, G118-1490, L119-L490, G120-L490, C121-L490, T122-L490, V123-L490, D124-L490, V125-L490, N126-L490, H127-L490, F128-L490, G129-L490, A130-L490, H131-L490, V132-L490, R133-L490, R134-1490, P135-L490, V136-L490, A137-L490, A138-L490, L139-L490, L140-L490, A141-L490, A142-L490, L143-L490, P144-L490, V145-L490, R146-L490, P147-L490, P148-L490, A149-L490, A150-L490, A151-L490, G152-L490, L153-L490, P154-L490, A155-L490, G156-L490, P157-L490, R158-L490, L159-L490, Q160-L490, A161-L490, G162-L490, R163-L490, G164-L490, G165-L490, R166-L490, R167-L490, G168-L490, L169-L490, L170-L490, L171-L490, C172-L490, G173-L490, C174-L490, C175-L490, P176-L490, G177-1490, G178-L490, N179-L490, L180-L490, S181-L490, N182-L490, L183-L490, M184-L490, S185-L490, L186-L490, L187-L490, V188-L490, D189-L490, G190-L490, D191-L490, M192-L490, N193-L490, L194-L490, R195-L490, R196-L490, A197-L490, A198-L490, L199-L490, L200-L490, A201-L490, L202-L490, S203-L490, S204-L490, D205-L490, V206-L490, G207-L490, S208-L490, A209-L490, Q210-L490, T211-L490, S212-L490, T213-L490, P214-L490, G215-L490, L216-L490, A217-L490, V218-L490, S219-L490, P220-L490, F221-L490, H222-L490, L223-L490, Y224-L490, S225-L490, T226-L490, Y227-L490, K228-L490, K229-L490, K230-L490, V231-L490, S232-L490, W233-L490, L234-L490, F235-L490, D236-L490, S237-L490, K238-L490, L239-L490, V240-L490, L241-L490, 1242-L490, S243-L490, A244-L490, H245-L490, S246-L490, L247-L490, F248-L490, C249-L490, S250-L490, I251-L490, I252-L490, M253-L490, T254-L490, I255-L490, S256-L490, S257-L490, T258-L490, L259-L490, L260-L490, A261-L490, L262-L490, V263-L490, L264-L490, M265-L490, P266-L490, L267-L490, C268-L490, L269-L490, W270-L490, I271-L490, Y272-L490, S273-L490, W274-L490, A275-L490, W276-L490, I277-L490, N278-L490, T279-L490, P280-L490, I281-L490, V282-L490, Q283-L490, L284-L490, L285-L490, P286-L490, L287-L490, G288-L490, T289-L490, V290-L490, T291-L490, L292-L490, T293-L490, L294-L490, C295-L490, S296-L490, T297-L490, L298-L490, I299-L490, P300-L490, I301-L490, G302-L303-L490, G304-L490, V305-L490, F306-L490, 1307-L490, R308-L490, Y309-L490, K310-L490, Y311-L490, S312-L490, R313-L490, V314-L490, A315-L490, D316-L490, Y317-L490, I318-L490, V319-L490, K320-L490, V321-L490, S322-L490, L323-L490, W324-L490, S325-L490, L326-L490, L327-L490, V328-L490, T329-L490, L330-L490, V331-L490, V332-L490, L333-L490, F334-L490, I335-L490, M336-L490, T337-L490, G338-L490, T339-L490, M340-L490, L341-L490, G342-L490, P343-L490, E344-L490, L345-L490, L346-L490, A347-L490, S338-L490, I349-L490, P350-L490, A351 -L490, A352-L490, V353-L490, Y354-L490, V355-L490, I356-L490, A357-L490, I358-L490, F359-L490, M360-L490, P361-L490, L362-L490, A363-L490, A364-L490, Y365-L490, A366-L490, S367-L490, G368-L490, Y369-L490, G370-L490, L371-L490, A372-L490, T373-L490, L374-L490, F375-L490, H376-L490, L377-L490, P378-L490, P379-L490, N380-L490, C381-L490, K382-L490, R383-L490, T384-L490, V385-L490, C386-L490, L387-L490, E388-L490, T389-L490, G390-L490, S391-L490, Q392-L490, N393-L490, V394-L490, Q395-L490, L396-L490, C397-L490, T398-L490, A399-L490, I400-L490, L401-L490, K402-L490, L403-L490, A404-L490, F405-L490, P406-L490, P407-L490, Q408-L490, F409-L490, I410-L490, G411-L490, S412-L490, M413-L490, Y414-L490, M415-L490, F416-L490, P417-L490, L418-L490, L419-L490, Y420-L490, A421-L490, L422-L490, F423-L490, Q424-L490, S425-L490, A426-L490, E427-L490, A428-L490, G429-L490, I430-L490, F431 -L490, V432-L490, L433-L490, I434-L490, Y435-L490, K436-L490, M437-L490, Y438-L490, G439, L490, S440-L490, E441-L490, M442-L490, L443-L490, H444-L490, K445-L490, R446-L490, D447-L490, P448-L490, L449-L490, D450-L490, E451-L490, D452-L490, E453-L490, D454-L490, T455-L490, D456-L490, I457-L490, S458-L490, Y459-L490, K460-L490, K461-L490, L462-L490, K463-L490, E464-L490, E465-L490, E466-L490, M467-L490, A468-L490, D469-L490, T470-L490, S471-L490, Y472-L490, G473-L490, T474-L490, V475-L490, K476-L490, A477-L490, E478-L490, N479-L490, I480-L490, I481-L490, M482-L490, M483-L490, and/or E484-L490 of SEQ ID NO:109. Polynucleotide sequences encoding these polypeptides are also provided. The present invention also encompasses the use of these N-terminal AC024191 deletion polypeptides as immunogenic and/or antigenic epitopes as described elsewhere herein.

In preferred embodiments, the following C-terminal AC024191 deletion polypeptides are encompassed by the present invention: M1-L490, M1-S489, M1-T488, M1-Q487, M1-A486, M1-T485, M1-E484, M1-M483, M1-M482, M1-I481, M1-I480, M1-N479, M1-E478, M1-A477, M1-K476, M1-V475, M1-T474, M1-G473, M1-Y472, M1-S471, M1-T470, M1-D469, M1-A468, M1-M467, M1-E466, M1-E465, M1-E464, M1-K463, M1-L462, M1-K461, M1-K460, M1-Y459, M1-S458, M1-1457, M1-D456, M1-T455, M1-D454, M1-E453, M1-D452, M1-E451, M1-D450, M1-L449, M1-P448, M1-D447, M1-R446, M1-K445, M1-H444, M1-L443, M1-M442, M1-E441, M1-S440, M1-G439, M1-Y438, M1-M437, M1-K436, M1-Y435, M1-I434, M1-L433, M1-V432, M1-F431, M1-I430, M1-G429, M1-A428, M1-E427, M1-A426, M1-S425, M1-Q424, M1-F423, M1-L422, M1-A421, M1-Y420, M1-L419, M1-L418, M1-P417, M1-F416, M1-M415, M1-Y414, M1-M413, M1-S412, M1-G411, M1-I410, M1-F409, M1-Q408, M1-P407, M1-P406, M1-F405, M1-A404, M1-L403, M1-K402, M1-L401, M1-I400, M1-A399, M1-T398, M1-C397, M1-L396, M1-Q395, M1-V394, M1-N393, M1-Q392, M1-S391, M1-G390, M1-T389, M1-E388, M1-L387, M1-C386, M1-V385, M1-T384, M1-R383, M1-K382, M1-C381, M1-N380, M1-P379, M1-P378, M1-L377, M1-H376, M1-F375, M1-L374, M1-T373, M1-A372, M1-L371, M1-G370, M1-Y369, M1-G368, M1-S367, M1-A366, M1-Y365, M1-A364, M1-A363, M1-L362, MI-P361, M1-M360, M1-F359, M1-I358, M1-A357, M1-I356, M1-V355, M1-Y354, M1-V353, M1-A352, M1-A351, M1-P350, M1-I349, M1-S348, M1-A347, M1-L346, M1-L345, M1-E344, M1-P343, M1-G342, M1-L341, M1-M340, M1-T339, M1-G338, M1-T337, M1-M336, M1-I335, M1-F334, M1-L333, M1-V332, M1-V331, M1-L330, M1-T329, M1-V328, M1-L327, M1-L326, M1-S325, M1-W324, M1-L323, M1-S322, M1-V321, M1-K320, M1-V319, M1-I318, M1-Y317, M1-D316, M1-A315, M1-V314, M1-R313, M1-S312, M1-Y311, M1-K310, M1-Y309, M1-R308, M1-1307, M1-F306, M1-V305, M1-G304, M1-L303, M1-G302, M1-I301, M1-P300, M1-I299, M1-L298, M1-T297, M1-S296, M1-C295, M1-L294, M1-T293, M1-L292, M1-T291, M1-V290, M1-T289, M1-G288, M1-L287, M1-P286, M1-L285, M1-L284, M1-Q283, M1-V282, M1-I281, M1-P280, M1-T279, M1-N278, M1-I277, M1-W276, M1-A275, M1-W274, M1-S273, M1-Y272, M1-I271, M1-W270, M1-L269, M1-C268, M1-L267, M1-P266, M1-M265, M1-L264, M1-V263, M1-L262, M1-A261, M1-L260, M1-L259, M1-T258, M1-S257, M1-S256, M1-I255, M1-T254, M1-M253, M1-I252, M1-I251, M1-S250, M1-C249, M1-F248, M1-L247, M1-S246, M1-H245, M1-A244, M1-S243, M1-I242, M1-L241, M1-V240, M1-L239, M1-K238, M1-S237, M1-D236, M1-F235, M1-L234, M1-W233, M1-S232, M1-V231, M1-K230, M1-K229, M1-K228, M1-Y227, M1-T226, M1-S225, M1-Y224, M1-L223, M1-H222, M1-F221, M1-P220, M1-S219, M1-V218, M1-A217, M1-L216, M1-G215, M1-P214, M1-T213, M1-S212, M1-T211, M1-Q210, M1-A209, M1-S208, M1-G207, M1-V206, M1-D205, M1-S204, M1-S203, M1-L202, M1-A201, M1-L200, M1-L199, M1-A198, M1-A197, M1-R196, M1-R195, M1-L194, M1-N193, M1-M192, M1-D191, M1-G190, M1-D189, M1-V188, M1-L187, M1-L186, M1-S185, M1-M184, M1-L183, M1-N182, M1-S181, M1-L180, M1-N179, M1-G178, M1-G177, M1-P176, M1-C175, M1-C174, M1-G173, M1-C172, M1-L171, M1-L170, M1-L169, M1-G168, M1-R167, M1-R166, M1-G165, M1-G164, M1-R163, M1-G162, M1-A161, M1-Q160, M1-L159, M1-R158, M1-P157, M1-G156, M1-A155, M1-P154, M1-L153, M1-G152, M1-A151, M1-A150, M1-A149, M1-P148, M1-P147, M1-R146, M1-V145, M1-P144, M1-L143, M1-A142, M1-A141, M1-L140, M1-L139, M1-A138, M1-A137, M1-V136, M1-P135, M1-R134, M1-R133, M1-V132, M1-H131, M1-A130, M1-G129, M1-F128, M1-H127, M1-N126, M1-V125, M1-D124, M1-V123, M1-T122, M1-C121, M1-G120, M1-L119, M1-G118, M1-L117, M1-M116, M1-T115, M1-I114, M1-C113, M1-L112, M1-R111, M1-R110, M1-G109, M1-R108, M1-V107, M1-R106, M1-E105, M1-A104, M1-R103, M1-P102, M1-E101, M1-A100, M1-A99, M1-H98, M1-G97, M1-L96, M1-V95, M1-P94, M1-A93, M1-R92, M1-P91, M1-A90, M1-G89, M1-L88, M1-A87, M1-S86, M1-P85, M1-V84, M1-P83, M1-F82, M1-P81, M1-R80, M1-P79, M1-E78, M1-G77, M1-R76, M1-R75, M1-G74, M1-R73, M1-P72, M1-R71, M1-Q70, M1-D69, M1-H68, M1-A67, M1-G66, M1-P65, M1-D64, M1-S63, M1-H62, M1-P61, M1-R60, M1-A59, M1-S58, M1-A57, M1-S56, M1-A55, M1-R54, M1-Y53, M1-R52, M1-G51, M1-S50, M1-A49, M1-S48, M1-G47, M1-L46, M1-A45, M1-P44, M1-G43, M1-A42, M1-S41, M1-S40, M1-A39, M1-P38, M1-A37, M1-L36, M1-A35, M1-L34, M1-N33, M1-T32, M1-G31, M1-P30, M1-G29, M1-L28, M1-S27, M1-S26, M1-A25, M1-N24, M1-P23, M1-A22, M1-L21, M1-T20, M1-Y19, M1-N18, M1-D17, M1-R16, M1-L15, M1-L14, M1-P13, M1-A12, M1-F11, M1-L10, M1-L9, M1-T8, and/or M1-V7 of SEQ ID NO:109. Polynucleotide sequences encoding these polypeptides are also provided. The present invention also encompasses the use of these C-terminal AC024191 deletion polypeptides as immunogenic and/or antigenic epitopes as described elsewhere herein.

### Features of the Polypeptide Encoded by Gene No:78

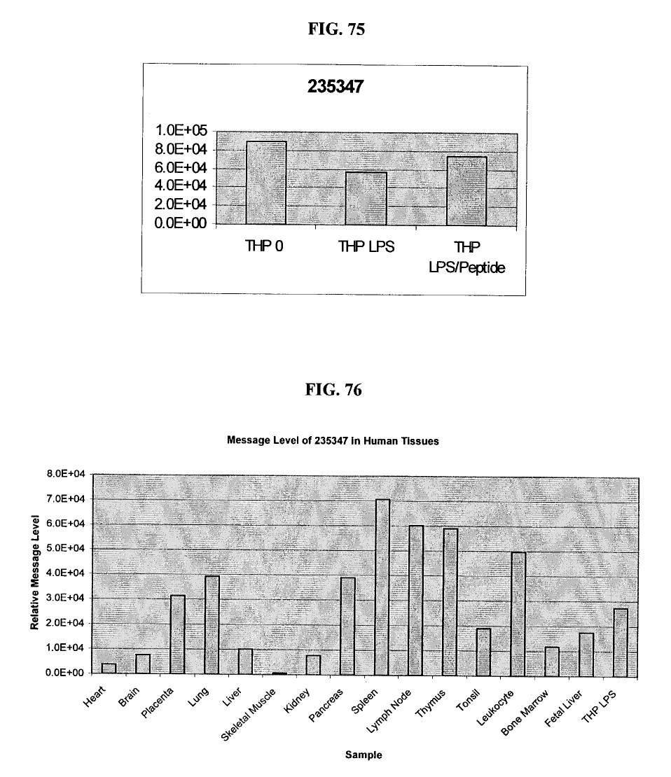

In confirmation that the 235347 (SEQ ID NO:78; SEQ ID NO: 282; Table II) polynucleotide and/or its encoded polypeptide are involved in the NF-kB pathway, real-time PCR analyses was used to show that 235347 expression is NF-kB-dependent, as shown in FIG. 75. 235347 was expressed in unstimulated THP-1 monocytes as a control. In response to stimulation with LPS, steady-state levels of 235347 mRNA increased. This increase in expression was specifically increased by inclusion of the selective NF-kB inhibitor, BMS-205820.

In an effort to identify additional associations of the 235347 polynucleotide and/or its encoded polypeptide with the NF-kB pathway in other human tissues, RT-PCR was performed on a variety of tissues. The results of these experiments indicate that 235347 mRNA is expressed at predominately high levels in spleen, lymph node, thymus, leukocyte, and to a lesser extent in lung, pancreas, placenta, other tissues as shown (see FIG. 76). The increased expression levels in immune tissues is consistent with the 235347 representing a NFkB modulated polynucleotide and polypeptide.

The confirmation that the expression of the 235347 polynucleotide and encoded peptide are inhibited by NFkB suggests that agonists directed against the 235347 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the 235347 polynucleotide and/or encoded peptide are useful for decreasing NF-kB activity, decreasing apoptotic events, and/or increasing IkBa expression or activity levels.

The 235347 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The 235347 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The expression in spleen, lymph node, thymus, leukocyte tissue, in combination with its association with the NFkB pathway suggests the 235347 polynucleotides and polypeptides, and particularly agonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scieroderma, and modulating cytokine production, antigen presentation, or other processes, such as for boosting immune responses.

The expression of 235347 transcripts in lung tissue, in combination with its association with the NFkB pathway suggests the potential utility for 235347 polynucleotides and polypeptides, and particularly agonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

The expression in pancreas, in combination with its association with the NFkB pathway suggests the 235347 polynucleotides and polypeptides, and particularly agonists, may be useful in treating, diagnosing, prognosing, and/or preventing pancreatic, in addition to metabolic and gastrointestinal disorders. In preferred embodiments, 262 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

In preferred embodiments, the following N-terminal clone 235347 deletion polypeptides are encompassed by the present invention: M1-N645, W2-N645, I3-N645, Q4-N645, V5-N645, R6-N645, T7-N645, 18-N645, D9-N645, G10-N645, S11-N645, K12-N645, T13-N645, C14-N645, T15-N645, 116-N645, E17-N645, D18-N645, V19-N645, S20-N645, R21-N645, K22-N645, A23-N645, T24-N645, I25-N645, E26-N645, E27-N645, L28-N645, R29-N645, E30-N645, R31-N645, V32-N645, W33-N645, A34-N645, L35-N645, F36-N645, D37-N645, V38-N645, R39-N645, P40-N645, E41-N645, C42-N645, Q43-N645, R44-N645, L45-N645, F46-N645, Y47-N645, R48-N645, G49-N645, K50-N645, Q5 I-N645, L52-N645, E53-N645, N54-N645, G55-N645, Y56-N645, T57-N645, L58-N645, F59-N645, D60-N645, Y61-N645, D62-N645, V63-N645, G64-N645, L65-N645, N66-N645, D67-N645, I68-N645, I69-N645, Q70-N645, L71-N645, L72-N645, V73-N645, R74-N645, P75-N645, D76-N645, P77-N645, D78-N645, H79-N645, L80-N645, P81-N645, G82-N645, T83-N645, S84-N645, T85-N645, Q86-N645, I87-N645, E88-N645, A89-N645, K90-N645, P91-N645, C92-N645, S93-N645, N94-N645, S95-N645, P96-N645, P97-N645, K98-N645, V99-N645, K100-N645, K101-N645, A102-N645, P103-N645, R104-N645, V105-N645, G106-N645, P107-N645, S108-N645, N109-N645, Q110-N645, P111-N645, S112-N645, T113-N645, S114-N645, A115-N645, R116-N645, A117-N645, R118-N645, L119-N645, I120-N645, D121-N645, P122-N645, G123-N645, F124-N645, G125-N645, I126-N645, Y127-N645, K128-N645, V129-N645, N130-N645, E131-N645, L132-N645, V133-N645, D134-N645, A135-N645, R136-N645, D137-N645, V138-N645, G139-N645, L140-N645, G141-N645, A142-N645, W143-N645, F144-N645, E145-N645, A146-N645, H147-N645, I148-N645, H149-N645, S150-N645, V151-N645, T152-N645, R153-N645, A154-N645, S155-N645, D156-N645, G157-N645, Q158-N645, S159-N645, R160-N645, G161-N645, K162-N645, T163-N645, P164-N645, L165-N645, K166-N645, N167-N645, G168-N645, S169-N645, S170-N645, C171-N645, K172-N645, R173-N645, T174-N645, N175-N645, G176-N645, N177-N645, 1178-N645, K179-N645, H180-N645, K181-N645, S182-N645, K183-N645, E184-N645, N185-N645, T186-N645, N187-N645, K188-N645, L189-N645, D190-N645, S191-N645, V192-N645, P193-N645, S194-N645, T195-N645, S196-N645, N197-N645, S198-N645, D199-N645, C200-N645, V201-N645, A202-N645, A203-N645, D204-N645, E205-N645, D206-N645, V207-N645, I208-N645, Y209-N645, H210-N645, I211-N645, Q212-N645, Y213-N645, D214-N645, E215-N645, Y216-N645, P217-N645, E218-N645, S219-N645, G220-N645, T221-N645, L222-N645, E223-N645, M224-N645, N225-N645, V226-N645, K227-N645, D228-N645, L229-N645, R230-N645, P231-N645, R232-N645, A233-N645, R234-N645, T235-N645, 1236-N645, L237-N645, K238-N645, W239-N645, N240-N645, E241-N645, L242-N645, N243-N645, V244-N645, G245-N645, D246-N645, V247-N645, V248-N645, M249-N645, V250-N645, N251-N645, Y252-N645, N253-N645, V254-N645, E255-N645, S256-N645, P257-N645, G258-N645, Q259-N645, R260-N645, G261-N645, F262-N645, W263-N645, F264-N645, D265-N645, A266-N645, E267-N645, I268-N645, T269-N645, T270-N645, L271-N645, K272-N645, T273-N645, I274-N645, S275-N645, R276-N645, T277-N645, K278-N645, K279-N645, E280-N645, L281-N645, R282-N645, V283-N645, K284-N645, I285-N645, F286-N645, L287-N645, G288-N645, G289-N645, S290-N645, E291-N645, G292-N645, T293-N645, L294-N645, N295-N645, D296-N645, C297-N645, K298-N645, I299-N645, I300-N645, S301-N645, V302-N645, D303-N645, E304-N645, I305-N645, F306-N645, K307-N645, 1308-N645, E309-N645, R310-N645, P311-N645, G312-N645, A313-N645, H314-N645, P315-N645, L316-N645, S317-N645, F318-N645, A319-N645, D320-N645, G321-N645, K322-N645, F323-N645, L324-N645, R325-N645, R326-N645, N327-N645, D328-N645, P329-N645, E330-N645, C331-N645, D332-N645, L333-N645, C334-N645, G335-N645, G336-N645, D337-N645, P338-N645, E339-N645, K340-N645, K341-N645, C342-N645, H343-N645, S344-N645, C345-N645, S346-N645, C347-N645, R348-N645, V349-N645, C350-N645, G351-N645, G352-N645, K353-N645, H354-N645, E355-N645, P356-N645, N357-N645, M358-N645, Q359-N645, L360-N645, L361-N645, C362-N645, D363-N645, E364-N645, C365-N645, N366-N645, V367-N645, A368-N645, Y369-N645, H370-N645, I371-N645, Y372-N645, C373-N645, L374-N645, N375-N645, P376-N645, P377-N645, L378-N645, D379-N645, K380-N645, V381-N645, P382-N645, E383-N645, E384-N645, E385-N645, Y386-N645, W387-N645, Y388-N645, C389-N645, P390-N645, S391-N645, C392-N645, K393-N645, T394-N645, D395-N645, S396-N645, S397-N645, E398-N645, V399-N645, V400-N645, K401-N645, A402-N645, G403-N645, E404-N645, R405-N645, L406-N645, K407-N645, M408-N645, S409-N645, K410-N645, K41 1-N645, K412-N645, A413-N645, K414-N645, M415-N645, P416-N645, S417-N645, A418-N645, S419-N645, T420-N645, E421-N645, S422-N645, R423-N645, R424-N645, D425-N645, W426-N645, G427-N645, R428-N645, G429-N645, M430-N645, A431-N645, C432-N645, V433-N645, G434-N645, R435-N645, T436-N645, R437-N645, E438-N645, C439-N645, T440-N645, I441-N645, V442-N645, P443-N645, S444-N645, N445-N645, H446-N645, Y447-N645, G448-N645, P449-N645, I450-N645, P451-N645, G452-N645, I453-N645, P454-N645, V455-N645, G456-N645, S457-N645, T458-N645, W459-N645, R460-N645, F461-N645, R462-N645, V463-N645, Q464-N645, V465-N645, S466-N645, E467-N645, A468-N645, G469-N645, V470-N645, H471-N645, R472-N645, P473-N645, H474-N645, V475-N645, G476-N645, G477-N645, I478-N645, H479-N645, G480-N645, R481-N645, S482-N645, N483-N645, D484-N645, G485-N645, A486-N645, Y487-N645, S488-N645, L489-N645, V490-N645, L491-N645, A492-N645, G493-N645, G494-N645, F495-N645, A496-N645, D497-N645, E498-N645, V499-N645, D500-N645, R501-N645, G502-N645, D503-N645, E504-N645, F505-N645, T506-N645, Y507-N645, T508-N645, G509-N645, S510-N645, G511-N645, G512-N645, K513-N645, N514-N645, L515-N645, A516-N645, G517-N645, N518-N645, K519-N645, R520-N645, I521-N645, G522-N645, A523-N645, P524-N645, S525-N645, A526-N645, D527-N645, Q528-N645, T529-N645, L530-N645, T531-N645, N532-N645, M533-N645, N534-N645, R535-N645, A536-N645, L537-N645, A538-N645, L539-N645, N540-N645, C541-N645, D542-N645, A543-N645, P544-N645, L545-N645, D546-N645, D547-N645, K548-N645, I549-N645, G550-N645, A551-N645, E552-N645, S553-N645, R554-N645, N555-N645, W556-N645, R557-N645, A558-N645, G559-N645, K560-N645, P561-N645, V562-N645, R563-N645, V564-N645, I565-N645, R566-N645, S567-N645, F568-N645, K569-N645, G570-N645, R571-N645, K572-N645, I573-N645, S574-N645, K575-N645, Y576-N645, A577-N645, P578-N645, E579-N645, E580-N645, G581-N645, N582-N645, R583-N645, Y584-N645, D585-N645, G586-N645, I587-N645, Y588-N645, K589-N645, V590-N645, V591-N645, K592-N645, Y593-N645, W594-N645, P595-N645, E596-N645, I597-N645, S598-N645, S599-N645, S600-N645, H601-N645, G602-N645, F603-N645, L604-N645, V605-N645, W606-N645, R607-N645, Y608-N645, L609-N645, L610-N645, R611-N645, R612-N645, D613-N645, D614-N645, V615-N645, E616-N645, P617-N645, A618-N645, P619-N645, W620-N645, T621-N645, S622-N645, E623-N645, G624-N645, I625-N645, E626-N645, R627-N645, S628-N645, R629-N645, R630-N645, L631-N645, C632-N645, L633-N645, R634-N645, G635-N645, L636-N645, C637-N645, L638-N645, and/or G639-N645 of SEQ ID NO:113. Polynucleotide sequences encoding these polypeptides are also provided. The present invention also encompasses the use of these N-terminal clone 235347 deletion polypeptides as immunogenic and/or antigenic epitopes as described elsewhere herein.

In preferred embodiments, the following C-terminal clone 235347 deletion polypeptides are encompassed by the present invention: M1-N645, M1-V644, M1-P643, M1-G642, M1-V641, M1-K640, M1-G639, M1-L638, M1-C637, M1-L636, M1-G635, M1-R634, M1-L633, M1-C632, M1-L631, M1-R630, M1-R629, M1-S628, M1-R627, M1-E626, M1-I625, M1-G624, M1-E623, M1-S622, M1-T621, M1-W620, M1-P619, M1-A618, M1-P617, M1-E616, M1-V615, M1-D614, M1-D613, M1 -R612, M1-R611, M1-L610, M1-L609, M1-Y608, M1-R607, M1-W606, M1-V605, M1-L604, M1-F603, M1-G602, M1-H601, M1-S600, M1-S599, M1-S598, M1-I597, M1-E596, M1-P595, M1-W594, M1-Y593, M1-K592, M1-V591, M1-V590, M1-K589, M1-Y588, M1-I587, M1-G586, M1-D585, M1-Y584, M1-R583, M1-N582, M1-G581, M1-E580, M1-E579, M1-P578, M1-A577, M1-Y576, M1-K575, M1-S574, M1-I573, M1-K572, M1-R571, M1-G570, M1-K569, M1-F568, M1-S567, M1-R566, M1-I565, M1-V564, M1-R563, M1-V562, M1-P561, M1-K560, M1-G559, M1-A558, M1-R557, M1-W556, M1-N555, M1-R554, M1-S553, M1-E552, M1-A551, M1-G550, M1-I549, M1-K548, M1-D547, M1-D546, M1-L545, M1-P544, M1-A543, M1-D542, M1-C541, M1-N540, M1-L539, M1-A538, M1-L537, M1-A536, M1-R535, M1-N534, M1-M533, M1-N532, M1-T531, M1-L530, M1-T529, M1-Q528, M1-D527, M1-A526, M1-S525, M1-P524, M1-A523, M1-G522, M1-I521, M1-R520, M1-K519, M1-N518, M1-G517, M1-A516, M1-L515, M1-N514, M1-K513, M1-G512, M1-G511, M1-S510, M1-G509, M1-T508, M1-Y507, M1-T506, M1-F505, M1-E504, M1-D503, M1-G502, M1-R501, M1-D500, M1-V499, M1-E498, M1-D497, M1-A496, M1-F495, M1-G494, M1-G493, M1-A492, M1-L491, M1-V490, M1-L489, M1-S488, M1-Y487, M1-A486, M1-G485, M1-D484, M1-N483, M1-S482, M1-R481, M1-G480, M1-H479, M1-I478, M1-G477, M1-G476, M1-V475, M1-H474, M1-P473, M1-R472, M1-H471, M1-V470, M1-G469, M1-A468, M1-E467, M1-S466, M1-V465, M1-Q464, M1-V463, M1-R462, M1-F461, M1-R460, M1-W459, M1-T458, M1-S457, M1-G456, M1-V455, M1-P454, M1-I453, M1-G452, M1-P451, M1-1450, M1-P449, M1-G448, M1-Y447, M1-H446, M1-N445, M1-S444, M1-P443, M1-V442, M1-I441, M1-T440, M1-C439, M1-E438, M1-R437, M1-T436, M1-R435, M1-G434, M1-V433, M1-C432, M1-A431, M1-M430, M1-G429, M1-R428, M1-G427, M1-W426, M1-D425, M1-R424, M1-R423, M1-S422, M1-E421, M1-T420, M1-S419, M1-A418, M1-S417, M1-P416, M1-M415, M1-K414, M1-A413, M1-K412, M1-K411, M1-K410, M1-S409, M1-M408, M1-K407, M1-L406, M1-R405, M1-E404, M1-G403, M1-A402, M1-K401, M1-V400, M1-V399, M1-E398, M1-S397, M1-S396, M1-D395, M1-T394, M1-K393, M1-C392, M1-S391, M1-P390, M1-C389, M1-Y388, M1-W387, M1-Y386, M1-E385, M1-E384, M1-E383, M1-P382, M1-V381, M1-K380, M1-D379, M1-L378, M 1-P377, M1-P376, M1-N375, M1-L374, M1-C373, M1-Y372, M1-I371, M1-H370, M1-Y369, M1-A368, M1-V367, M1-N366, M1-C365, M1-E364, M1-D363, M1-C362, M1-L361, M1-L360, M1-Q359, M1-M358, M1-N357, M1-P356, M1-E355, M1-H354, M1-K353, M1-G352, M1-G351, M1-C350, M1-V349, M1-R348, M1-C347, M1-S346, M1-C345, M1-S344, M1-H343, M1-C342, M1-K341, M1-K340, M1-E339, M1-P338, M1-D337, M1-G336, M1-G335, M1-C334, M1-L333, M1-D332, M1-C331, M1-E330, M1-P329, M1-D328, M1-N327, M1-R326, M1-R325, M1-L324, M1-F323, M1-K322, M1-G321, M1-D320, M1-A319, M1-F318, M1-S317, M1-L316, M1-P315, M1-H314, M1-A313, M1-G312, M1-P311, M1-R310, M1-E309, M1-I308, M1-K307, M1-F306, M1-I305, M1-E304, M1-D303, M1-V302, M1-S301, M1-I300, M1-I299, M1-K298, M1-C297, M1-D296, M1-N295, M1-L294, M1-T293, M1-G292, M1-E291, M1-S290, M1-G289, M1-G288, M1-L287, M1-F286, M1-I285, M1-K284, M1-V283, M1-R282, M1-L281, M1-E280, M1-K279, M1-K278, M1-T277, M1-R276, M1-S275, M1-1274, M1-T273, M1-K272, M1-L271, M1-T270, M1-T269, M1-1268, M1-E267, M1-A266, M1-D265, M1-F264, M1-W263, M1-F262, M1-G261, M1-R260, M1-Q259, M1-G258, M1-P257, M1-S256, M1-E255, M1-V254, M1-N253, M1-Y252, M1-N251, M1-V250, M1-M249, M1-V248, M1-V247, M1-D246, M1-G245, M1-V244, M1-N243, M1-L242, M1-E241, M1-N240, M1-W239, M1-K238, M1-L237, M1-I236, M1-T235, M1-R234, M1-A233, M1-R232, M1-P231, M1-R230, M1-L229, M1-D228, M1-K227, M1-V226, M1-N225, M1-M224, M1-E223, M1-L222, M1-T221, M1-G220, M1-S219, M1-E218, M1-P217, M1-Y216, M1-E215, M1-D214, M1-Y213, M1-Q212, M1-I211, M1-H210, M1-Y209, M1-I208, M1-V207, M1-D206, M1-E205, M1-D204, M1-A203, M1-A202, M1-V201, M1-C200, M1-D199, M1-S198, M1-N197, M1-S196, M1-T195, M1-S194, M1-P193, M1-V192, M1-S191, M1-D190, M1-L189, M1-K188, M1-N187, M1-T186, M1-N185, M1-E184, M1-K183, M1-S182, M1-K181, M1-H180, M1-K179, M1-I178, M1-N177, M1-G176, M1-N175, M1-T174, M1-R173, M1-K172, M1-C171, M1-S170, M1-S169, M1-G168, M1-N167, M1-K166, M1-L165, M1-P164, M1-T163, M1-K162, M1-G161, M1-R160, M1-S159, M1-Q158, M1-G157, M1-D156, M1-S155, M1-A154, M1-R153, M1-T152, M1-V151, M1-S150, M1-H149, M1-I148, M1-H147, M1-A146, M1-E145, M1-F144, M1-W143, M1-A142, M1-G141, M1-L140, M1-G139, M1-V138, M1-D137, M1-R136, M1-A135, M1-D134, M1-V133, M1-L132, M1-E131, M1-N130, M1-V129, M1-K128, M1-Y127, M1-I126, M1-G125, M1-F124, M1-G123, M1-P122, M1-D121, M1-I120, M1-L119, M1-R118, M1-A117, M1-R116, M1-A115, M1-S114, M1-T113, M1-S112, M1-P111, M1-Q110, M1-N109, M1-S108, M1-P107, M1-G106, M1-V105, M1-R104, M1-P103, M1-A102, M1-K101, M1-K100, M1-V99, M1-K98, M1-P97, M1-P96, M1-S95, M1-N94, M1-S93, M1-C92, M1-P91, M1-K90, M1-A89, M1-E88, M1-187, M1-Q86, M1-T85, M1-S84, M1-T83, M1-G82, M1-P81, M1-L80, M1-H79, M1-D78, M1-P77, M1-D76, M1-P75, M1-R74, M1-V73, M1-L72, M1-L71, M1-Q70, M1-I69, M1-I68, M1-D67, M1-N66, M1-L65, M1-G64, M1-V63, M1-D62, M1-Y61, M1-D60, M1-F59, M1-L58, M1-T57, M1-Y56, M1-G55, M1-N54, M1-E53, M1-L52, M1-Q51, M1-K50, M1-G49, M1-R48, M1-Y47, M1-F46, M1-L45, M1-R44, M1-Q43, M1-C42, M1-E41, M1-P40, M1-R39, M1-V38, M1-D37, M1-F36, M1-L35, M1-A34, M1-W33, M1-V32, M1-R31, M1-E30, M1-R29, M1-L28, M1-E27, M1-E26, M1-125, M1-T24, M1-A23, M1-K22, M1-R21, M1-S20, M1-V19, M1-D18, M1-E17, M1-I16, M1-T15, M1-C14, M1-T13, M1-K12, M1-S11, M1-G10, M1-D9, M1-18, and/or M1-T7 of SEQ ID NO: 113. Polynucleotide sequences encoding these polypeptides are also provided. The present invention also encompasses the use of these C-terminal clone 235347 deletion polypeptides as immunogenic and/or antigenic epitopes as described elsewhere herein.

### Features of the Polypeptide Encoded by Gene No:81

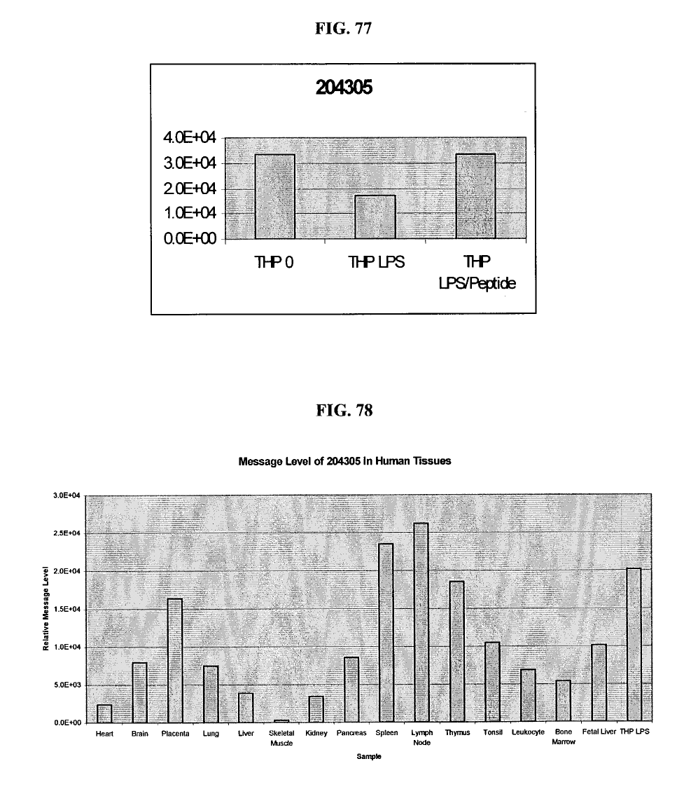

In confirmation that the 204305 (SEQ ID NO:81; Table II) polynucleotide and/or its encoded polypeptide are involved in the NF-kB pathway, real-time PCR analyses was used to show that 204305 expression is NF-kB-dependent, as shown in FIG. 77. 204305 was expressed in unstimulated THP-1 monocytes as a control. In response to stimulation with LPS, steady-state levels of 204305 mRNA increased. This increase in expression was specifically increased by inclusion of the selective NF-kB inhibitor, BMS-205820.

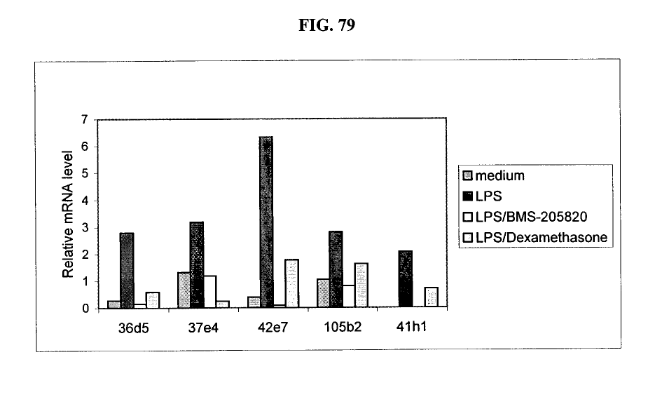

In an effort to identify additional associations of the 204305 polynucleotide and/or its encoded polypeptide with the NF-kB pathway in other human tissues, RT-PCR was performed on a variety of tissues. The results of these experiments indicate that 204305 mRNA is expressed at predominately high levels in lymph node, spleen, LPS treated THP cells; thymus, and to a lesser extent in placenta, tonsil, and other tissues as shown (see FIG. 78). The increased expression levels in immune tissues is consistent with the 204305 representing a NFkB modulated polynucleotide and polypeptide.

The confirmation that the expression of the 204305 polynucleotide and encoded peptide are inhibited by NFkB suggests that agonists directed against the 204305 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, agonists directed against the 204305 polynucleotide and/or encoded peptide are useful for decreasing NF-kB activity, decreasing apoptotic events, and/or increasing IkBa expression or activity levels.

The 204305 NFkB associated polynucleotide and polypeptide of the present invention, including agonists, and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, and EAE.

The 204305 NFkB associated polynucleotide and polypeptide of the present invention, including agonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., as described herein).

The expression in lymph node, spleen, LPS treated THP cells, thymus, in combination with its association with the NFkB pathway suggests the 204305 polynucleotides and polypeptides, and particularly agonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

In preferred embodiments, the following N-terminal clone 204305 deletion polypeptides are encompassed by the present invention: M1-I812, E2-I812, A3-I812, F4-I812, Q5-I812, E6-I812, L7-I812, R8-I812, K9-I812, P10-I812, S11-I812, A12-I812, R13-I812, L14-I812, E15-I812, C16-I812, D17-I812, H18-I812, C19-I812, S20-I812, F21-I812, R22-I812, G23-I812, T24-I812, D25-I812, Y26-I812, E27-I812, N28-I812, V29-I812, Q30-I812, I31-I812, H32-I812, M33-I812, G34-I812, T35-I812, I36-I812, H37-I812, P38-I812, E39-I812, F40-I812, C41-I812, D42-I812, E43-I821, M44-I812, D45-I812, A46-I812, G47-I812, G48-I812, L49-I812, G50-I812, K51-I812, M52-I812, 153-I812, F54-I812, Y55-I812, Q56-I812, K57-I812, S58-I812, A59-I812, K60-I812, L61-I812, F62-I812, H63-I812, C64-I812, H65-I812, K66-I812, C67-I812, F68-I812, F69-I812, T70-I812, S71-I812, K72-I812, M73-I812, Y74-I812, S75-I812, N76-I812, V77-I812, Y78-I812, Y79-I812, H80-I812, I81-I812, T82-I812, S83-I812, K84-I812, H85-I812, A86-I812, S87-I812, P88-I812, D89-I812, K90-I812, W91-I812, N92-I812, D93-I812, K94-I812, P95-I812, K96-I812, N97-I812, Q98-I812, L99-I812, N100-I812, K101-I812, E102-I812, T103-I812, D104-I812, P105-I812, V106-I812, K107-I812, S108-I812, P109-I812, P110-I812, L111-I812, P112-I812, E113-I812, H114-I812, Q115-I812, K116-I812, I117-I812, P118-I812, C119-I812, N120-I812, S121-I812, A122-I812, E123-I812, P124-I812, K125-I812, S126-I812, I127-I812, P128-I812, A129-I812, L130-I812, S131-I812, M132-I812, E133-I812, T134-I812, Q135-I812, K136-I812, L137-I812, G138-I812, S139-I812, V140-I812, L141-I812, S142-I812, P143-I812, E144-I812, S145-I812, P146-812, K147-I812, P148-I812, T149-I812, P150-I812, L151-I812, T152-I812, P153-I812, L154-I812, E155-I812, P156-I812, Q157-I812, K158-I812, P159-I812, G160-I812, S161-I812, V162-I812, V163-I812, S164-I812, P165-I812, E166-I812, L167-I812, Q168-I812, T169-I812, P170-I812, L171-I812, P172-I812, S173-I812, P174-I812, E175-I812, P176-I812, S177-I812, K178-I812, P179-I812, A180-I812, S181-I812, V182-I812, S183-I812, S184-I812, P185-I812, E186-I812, P187-I812, P188-I812, K189-I812, S190-I812, V191-I812, P192-I812, V193-I812, C194-I812, E195-I812, S196-I812, Q197-I812, K198-I812, L199-I812, A200-I812, P201-I812, V202-I812, P203-I812, S204-I812, P205-I812, E206-I812, P207-I812, Q208-I812, K209-I812, P210-I812, A211-I812, P212-I812, V213-I812, S214-I812, P215-I812, E216-I812, S217-I812, V218-I812, K219-I812, A220-I812, T221-I812, L222-I812, S223-I812, N224-I812, P225-I812, K226-I812, P227-I812, Q228-I812, K229-I812, Q230-I812, S231-I812, H232-I812, F233-I812, P234-I812, E235-I812, T236-I812, L237-I812, G238-I812, P239-I812, P240-I812, S241-I812, A242-I812, S243-I812, S244-I812, P245-I812, E246-I812, S247-I812, P248-I812, V249-I812, L250-I812, A251-I812, A252-I812, S253-I812, P254-I812, E255-I812, P256-I812, W257-I812, G258-I812, P259-I812, S260-I812, P261-I812, A262-I812, A263-I812, S264-I812, P265-I812, E266-I812, S267-I812, R268-I812, K269-I812, S270-I812, A271-I812, R272-I812, T273-I812, T274-I812, S275-I812, P276-I812, E277-I812, P278-I812, R279-I812, K280-I812, P281-I812, S282-I812, P283-I812, S284-I812, E285-I812, S286-I812, P287-I812, E288-I812, P289-I812, W290-I812, K291-I812, P292-I812, F293-I812, P294-I812, A295-I812, V296-I812, S297-I812, P298-I812, E299-I812, P300-I812, R301-I812, R302-I812, P303-I812, A304-I812, P305-I812, A306-I812, V307-I812, S308-I812, P309-I812, G310-I812, S311-I812, W312-I812, K313-I812, P314-I812, G315-I812, P316-I812, P317-I812, G318-I812, S319-I812, P320-I812, R321-I812, P322-I812, W323-I812, K324-I812, S325-I812, N326-I812, P327-I812, S328-I812, A329-I812, S330-I812, S331-I812, G332-I812, P333-I812, W334-I812, K335-I812, P336-I812, A337-I812, K338-I812, P339-I812, A340-I812, P341-I812, S342-I812, V343-I812, S344-I812, P345-I812, G346-I812, P347-I812, W348-I812, K349-I812, P350-I812, I351-I812, P352-I812, S353-I812, V354-I812, S355-I812, P356-I812, G357-I812, P358-I812, W359-I812, K360-I812, P361-I812, T362-I812, P363-I812, S364-I812, V365-I812, S366-I812, S367-I812, A368-I812, S369-I812, W370-I812, K371-I812, S372-I812, S373-I812, S374-I812, V375-I812, S376-I812, P377-I812, S378-I812, S379-I812, W380-I812, K381-I812, S382-I812, P383-I812, P384-I812, A385-I812, S386-I812, P387-I812, E388-I812, S389-I812, W390-I812, K391-I812, S392-I812, G393-I812, P394-I812, P395-I812, E396-I812, L397-I812, R398-I812, K399-I812, T400-I812, A401-I812, P402-I812, T403-I812, L404-I812, S405-I812, P406-I812, E407-I812, H408-I812, W409-I812, K410-I812, A411-I812, V412-I812, P413-I812, P414-I812, V415-I812, S416-I812, P417-I812, E418-I812, L419-I812, R420-I812, K421-I812, P422-I812, G423-I812, P424-I812, P425-I812, L426-I812, S427-I812, P428-I812, E429-I812, I430-I812, R431-I812, S432-I812, P433-I812, A434-I812, G435-I812, S436-I812, P437-I812, E438-I812, L439-I812, R440-I812, K441-I812, P442-I812, S443-I812, G444-I812, S445-I812, P446-I812, D447-I812, L448-I812, W449-I812, K450-I812, L451-I812, S452-I812, P453-I812, D454-I812, Q455-I812, R456-I812, K457-I812, T458-I812, S459-I812, P460-I812, A461-I812, S462-I812, L463-I812, D464-I812, F465-I812, P466-I812, E467-I812, S468-I812, Q469-I812, K470-I812, S471-I812, S472-I812, R473-I812, G474-I812, G475-I812, S476-I812, P477-I812, D478-I812, L479-I812, W480-I812, K481-I812, S482-I812, S483-I812, F484-I812, F485-I812, I486-I812, E487-I812, P488-I812, Q489-I812, K490-I812, P491-I812, V492-I812, F493-I812, P494-I812, E495-I812, T496-I812, R497-I812, K498-I812, P499-I812, G500-I812, P501-I812, S502-I812, G503-I812, P504-I812, S505-I812, E506-I812, S507-I812, P508-I812, K509-I812, A510-I812, A511-I812, S512-I812, D513-I812, I514-I812, W515-I812, K516-I812, P517-I812, V518-I812, L519-I812, S520-I812, I521-I812, D522-I812, T523-I812, E524-I812, P525-I812, R526-I812, K527-I812, P528-I812, A529-I812, L530-I812, F531-I812, P532-I812, E533-I812, P534-I812, A535-I812, K536-I812, T537-I812, A538-I812, P539-I812, P540-I812, A541-I812, S542-I812, P543-I812, E544-I812, A545-I812, R546-I812, K547-I812, R548-I812, A549-I812, L550-I812, F551-I812, P552-I812, E553-I812, P554-I812, R555-I812, K556-I812, H557-I812, A558-I812, L559-I812, F560-I812, P561-I812, E562-I812, L563-I812, P564-I812, K565-I812, S566-I812, A567-I812, L568-I812, F569-I812, S570-I812, E571-I812, S572-I812, Q573-I812, K574-I812, A575-I812, V576-I812, E577-I812, L578-I812, G579-I812, D580-I812, E581-I812, L582-I812, Q583-I812, I584-I812, D585-I812, A586-I812, I587-I812, D588-I812, D589-I812, Q590-I812, K591-I812, C592-I812, D593-I812, I594-I812, L595-I812, V596-I812, Q597-I812, E598-I812, E599-I812, L600-I812, L601-I812, A602-I812, S603-I812, P604-I812, K605-I812, K606-I812, L607-I812, L608-I812, E609-I812, D610-I812, T611-I812, L612-I812, F613-I812, P614-I812, S615-I812, S616-I812, K617-I812, K618-I812, L619-I812, K620-I812, K621-I812, D622-I812, N623-I812, Q624-I812, E625-I812, S626-I812, S627-I812, D628-I812, A629-I812, E630-I812, L631-I812, S632-I812, S633-I812, S634-I812, E635-I812, Y636-I812, I637-I812, K638-I812, T639-I812, D640-I812, L641-I812, D642-I812, A643-I812, M644-I812, D645-I812, I646-I812, K647-I812, G648-I812, Q649-I812, E650-I812, S651-I812, S652-I812, S653-I812, D654-I812, Q655-I812, E656-I812, Q657-I812, V658-I812, D659-I812, V660-I812, E661-I812, S662-I812, I663-I812, D664-I812, F665-I812, S666-I812, K667-I812, E668-I812, N669-I812, K670-I812, M671-I812, D672-I812, M673-I812, T674-I812, S675-I812, P676-I812, E677-I812, Q678-I812, S679-I812, R680-I812, N681-I812, V682-I812, L683-I812, Q684-I812, F685-I812, T686-I812, E687-I812, E688-I812, K689-I812, E690-I812, A691-I812, F692-I812, I693-I812, S694-I812, E695-I812, E696-I812, E697-I812, I698-I812, A699-I812, K700-I812, Y701-I812, M702-I812, K703-I812, R704-I812, G705-I812, K706-I812, G707-I812, K708-I812, Y709-I812, Y710-I812, C711-I812, K712-I812, I713-I812, C714-I812, C715-I812, C716-I812, R717-I812, A718-I812, M719-I812, K720-I812, K721-I812, G722-I812, A723-I812, V724-I812, L725-I812, H726-I812, H727-I812, L728-I812, V729-I812, N730-I812, K731-I812, H732-I812, N733-I812, V734-I812, H735-I812, S736-I812, P737-I812, Y738-I812, K739-I812, C740-I812, T741-I812, I742-I812, C743-I812, G744-I812, K745-I812, A746-I812, F747-I812, L748-I812, L749-I812, E750-I812, S751-I812, L752-I812, L753-I812, K754-I812, N755-I812, H756-I812, V757-I812, A758-I812, A759-I812, H760-I812, G761-I812, Q762-I812, S763-I812, L764-I812, L765-I812, K766-I812, C767-I812, P768-I812, R769-I812, C770-I812, N771-I812, F772-I812, E773-I812, S774-I812, N775-I812, F776-I812, P777-I812, R778-I812, G779-I812, F780-I812, K781-I812, K782-I812, H783-I812, L784-I812, T785-I812, H786-I812, C787-I812, Q788-I812, S789-I812, R790-I812, H791-I812, N792-I812, E793-I812, E794-I812, A795-I812, N796-I812, K797-I812, K798-I812, L799-I812, M800-I812, E801-I812, A802-I812, L803-I812, E804-I812, P805-I812, and/or P806-I812 of SEQ ID NO: 116. Polynucleotide sequences encoding these polypeptides are also provided. The present invention also encompasses the use of these N-terminal clone 204305 deletion polypeptides as immunogenic and/or antigenic epitopes as described elsewhere herein.

In preferred embodiments, the following C-terminal clone 204305 deletion polypeptides are encompassed by the present invention: M1-I812, M1-Q811, M1-Q810, M1-E809, M1-E808, M1-L807, M1-P806, M1-P805, M1-E804, M1-L803, M1-A802, M1-E801, M1-M800, M1-L799, M1-K798, M1-K797, M1-N796, M1-A795, M1-E794, M1-E793, M1-N792, M1-H791, M1-R790, M1-S789, M1-Q788, M1-C787, M1-H786, M1-T785, M1-L784, M1-H783, M1-K782, M1-K781, M1-F780, M1-G779, M1-R778, M1-P777, M1-F776, M1-N775, M1-S774, M1-E773, M1-F772, M1-N771, M1-C770, M1-R769, M1-P768, M1-C767, M1-K766, M1-L765, M1-L764, M1-S763, M1-Q762, M1-G761, M1-H760, M1-A759, M1-A758, M1-V757, M1-H756, M1-N755, M1-K754, M1-L753, M1-L752, M1-S751, M1-E750, M1-L749, M1-L748, M1-F747, M1-A746, M1-K745, M1-G744, M1-C743, M1-1742, M1-T741, M1-C740, M1-K739, M1-Y738, M1-P737, M1-S736, M1-H735, M1-V734, M1-N733, M1-H732, M1-K731, M1-N730, M1-V729, M1-L728, M1-H727, M1-H726, M1-L725, M1-V724, M1-A723, M1-G722, M1-K721, M1-K720, M1-M719, M1-A718, M1-R717, M1-C716, M1-C715, M1-C714, M1-I713, M1-K712, M1-C711, M I -Y710, M1-Y709, M1-K708, M1-G707, M1-K706, M1-G705, M1-R704, M1-K703, M1-M702, M1-Y701, M1-K700, M1-A699, M1-1698, M1-E697, M1-E696, M1-E695, M1-S694, M1-1693, M1-F692, M1-A691, M1-E690, M1-K689, M1-E688, M1-E687, M1-T686, M1-F685, M1-Q684, M1-L683, M1-V682, M1-N681, M1-R680, M1-S679, M1-Q678, M1-E677, M1-P676, M1-S675, M1-T674, M1-M673, M1-D672, M1-M671, M1-K670, M1-N669, M1-E668, M1-K667, M1-S666, M1-F665, M1-D664, M1-I663, M1-S662, M1-E661, M1-V660, M1-D659, M1-V658, M1-Q657, M1-E656, M1-Q655, M1-D654, M1-S653, M1-S652, M1-S651, M1-E650, M1-Q649, M1-G648, M1-K647, M1-I646, M1-D645, M1-M644, M1-A643, M1-D642, M1-L641, M1-D640, M1-T639, M1-K638, M1-I637, M1-Y636, M1-E635, M1-S634, M1-S633, M1-S632, M1-L631, M1-E630, M1-A629, M1-D628, M1-S627, M1-S626, M1-E625, M1-Q624, M1-N623, M1-D622, M1-K621, M1-K620, M1-L619, M1-K618, M1-K617, M1-S616, M1-S615, M1-P614, M1-F613, M1-L612, M1-T611, M1-D610, M1-E609, M1-L608, M1-L607, M1-K606, M1-K605, M1-P604, M1-S603, M1-A602, M1-L601, M1-L600, M1-E599, M1-E598, M1-Q597, M1-V596, M1-L595, M1-I594, M1-D593, M1-C592, M1-K591, M1-Q590, M1-D589, M1-D588, M1-I587, M1-A586, M1-D585, M1-I584, M1-Q583, M1-L582, M1-E581, M1-D580, M1-G579, M1-L578, M1-E577, M1-V576, M1-A575, M1-K574, M1-Q573, M1-S572, M1-E571, M1-S570, M1-F569, M1-L568, M1-A567, M1-S566, M1-K565, M1-P564, M1-L563, M1-E562, M1-P561, M1-F560, M1-L559, M1-A558, M1-H557, M1-K556, M1-R555, M1-P554, M1-E553, M1-P552, M1-F551, M1-L550, M1-A549, M1-R548, M1-K547, M1-R546, M1-A545, M1-E544, M1-P543, M1-S542, M1-A541, M1-P540, M1-P539, M1-A538, M1-T537, M1-K536, M1-A535, M1-P534, M1-E533, M1-P532, M1-F531, M1-L530, M1-A529, M1-P528, M1-K527, M1-R526, M1-P525, M1-E524, M1-T523, M1-D522, M1-I521, M1-S520, M1-L519, M1-V518, M1-P517, M1-K516, M1-W515, M1-I514, M1-D513, M1-S512, M1-A511, M1-A510, M1-K509, M1-P508, M1-S507, M1-E506, M1-S505, M1-P504, M1-G503, M1-S502, M1-P501, M1-G500, M1-P499, M1-K498, M1-R497, M1-T496, M1-E495, M1-P494, M1-F493, M1-V492, M1-P491, M1-K490, M1-Q489, M1-P488, M1-E487, M1-I486, M1-F485, M1-F484, M1-S483, M1-S482, M1-K481, M1-W480, M1-L479, M1-D478, M1-P477, M1-S476, M1-G475, M1-G474, M1-R473, M1-S472, M1-S471, M1-K470, M1-Q469, M1-S468, M1-E467, M1-P466, M1-F465, M1-D464, M1-L463, M1-S462, M1-A461, M1-P460, M1-S459, M1-T458, M1-K457, M1-R456, M1-Q455, M1-D454, M1-P453, M1-S452, M1-L451, M1-K450, M1-W449, M1-L448, M1-D447, M1-P446, M1-S445, M1-G444, M1-S443, M1-P442, M1-K441, M1-R440, M1-L439, M1-E438, M1-P437, M1-S436, M1-G435, M1-A434, M1-P433, M1-S432, M1-R431, M1-1430, M1-E429, M1-P428, M1-S427, M1-L426, M1-P425, M1-P424, M1-G423, M1-P422, M1-K421, M1-R420, M1-L419, M1-E418, M1-P417, M1-S416, M1-V415, M1-P414, M1-P413, M1-V412, M1-A411, M1-K410, M1-W409, M1-H408, M1-E407, M1-P406, M1-S405, M1-L404, M1-T403, M1-P402, M1-A401, M1-T400, M1-K399, M1-R398, M1-L397, M1-E396, M1-P395, M1-P394, M1-G393, M1-S392, M1-K391, M1-W390, M1-S389, M1-E388, M1-P387, M1-S386, M1-A385, M1-P384, M1-P383, M1-S382, M1-K381, M1-W380, M1-S379, M1-S378, M1-P377, M1-S376, M1-V375, M1-S374, M1-S373, M1-S372, M1-K371, M1-W370, M1-S369, M1-A368, M1-S367, M1-S366, M1-V365, M1-S364, M1-P363, M1-T362, M1-P361, M1-K360, M1-W359, M1-P358, M1-G357, M1-P356, M1-S355, M1-V354, M1-S353, M1-P352, M1-I351, M1-P350, M1-K349, M1-W348, M1-P347, M1-G346, M1-P345, M1-S344, M1-V343, M1-S342, M1-P341, M1-A340, M1-P339, M1-K338, M1-A337, M1-P336, M1-K335, M1-W334, M1-P333, M1-G332, M1-S331, M1-S330, M1-A329, M1-S328, M1-P327, M1-N326, M1-S325, M1-K324, M1-W323, M1-P322, M1-R321, M1-P320, M1-S319, M1-G318, M1-P317, M1-P316, M1-G315, M1-P314, M1-K313, M1-W312, M1-S311, M1-G310, M1-P309, M1-S308, M1-V307, M1-A306, M1-P305, M1-A304, M1-P303, M1-R302, M1-R301, M1-P300, M1-E299, M1-P298, M1-S297, M1-V296, M1-A295, M1-P294, M1-F293, M1-P292, M1-K291, M1-W290, M1-P289, M1-E288, M1-P287, M1-S286, M1-E285, M1-S284, M1-P283, M1-S282, M1-P281, M1-K280, M1-R279, M1-P278, M1-E277, M1-P276, M1-S275, M1-T274, M1-T273, M1-R272, M1-A271, M1-S270, M1-K269, M1-R268, M1-S267, M1-E266, M1-P265, M1-S264, M1-A263, M1-A262, M1-P261, M1-S260, M1-P259, M1-G258, M1-W257, M1-P256, M1-E255, M1-P254, M1-S253, M1-A252, M1-A251, M1-L250, M1-V249, M1-P248, M1-S247, M1-E246, M1-P245, M1-S244, M1-S243, M1-A242, M1-S241, M1-P240, M1-P239, M1-G238, M1-L237, M1-T236, M1-E235, M1-P234, M1-F233, M1-H232, M1-S231, M1-Q230, M1-K229, M1-Q228, M1-P227, M1-K226, M1-P225, M1-N224, M1-S223, M1-L222, M1-T221, M1-A220, M1-K219, M1-V218, M1-S217, M1-E216, M1-P215, M1-S214, M1-V213, M1-P212, M1-A211, M1-P210, M1-K209, M1-Q208, M1-P207, M1-E206, M1-P205, M1-S204, M1-P203, M1-V202, M1-P201, M1-A200, M1-L199, M1-K198, M1-Q197, M1-S196, M1-E195, M1-C194, M1-V193, M1-P192, M1-V191, M1-S190, M1-K189, M1-P188, M1-P187, M1-E186, M1-P185, M1-S184, M1-S183, M1-V182, M1-S181, M1-A180, M1-P179, M1-K178, M1-S177, M1-P176, M1-E175, M1-P174, M1-S173, M1-P172, M1-L171, M1-P170, M1-T169, M1-Q168, M1-L167, M1-E166, M1-P165, M1-S164, M1-V163, M1-V162, M1-S161, M1-G160, M1-P159, M1-K158, M1-Q157, M1-P156, M1-E155, M1-L154, M1-P153, M1-T152, M1-L151, M1-P150, M1-T149, M1-P148, M1-K147, M1-P146, M1-S145, M1-E144, M1-P143, M1-S142, M1-L141, M1-V140, M1-S139, M1-G138, M1-L137, M1-K136, M1-Q135, M1-T134, M1-E133, M1-M132, M1-S131, M1-L130, M1-A129, M1-P128, M1-I127, M1-S126, M1-K125, M1-P124, M1-E123, M1-A122, M1-S121, M1-N120, M1-C119, M1-P118, M1-I117, M1-K116, M1-Q115, M1-H114, M1-E113, M1-P112, M1-L111, M1-P110, M1-P109, M1-S108, M1-K107, M1-V106, M1-P105, M1-D104, M1-T103, M1-E102, M1-K101, M1-N100, M1-L99, M1-Q98, M1-N97, M1-K96, M1-P95, M1-K94, M1-D93, M1-N92, M1-W91, M1-K90, M1-D89, M1-P88, M1-S87, M1-A86, M1-H85, M1-K84, M1-S83, M1-T82, M1-I81, M1-H80, M1-Y79, M1-Y78, M1-V77, M1-N76, M1-S75, M1-Y74, M1-M73, M1-K72, M1-S71, M1-T70, M1-F69, M1-F68, M1-C67, M1-K66, M1-H65, M1-C64, M1-H63, M1-F62, M1-L61, M1-K60, M1-A59, M1-S58, M1-K57, M1-Q56, M1-Y55, M1-F54, M1-I53, M1-M52, M1-K51, M1-G50, M1-L49, M1-G48, M1-G47, M1-A46, M1-D45, M1-M44, M1-E43, M1-D42, M1-C41, M1-F40, M1-E39, M1-P38, M1-H37, M1-I36, M1-T35, M1-G34, M1-M33, M1-H32, M1-I31, M1-Q30, M1-V29, M1-N28, M1-E27, M1-Y26, M1-D25, M1-T24, M1-G23, M1-R22, M1-F21, M1-S20, M1-C19, M1-H18, M1-D17, M1-C16, M1-E15, M1-L14, M1-R13, M1-A12, M1-S11, M1-P10, M1-K9, M1-R8, and/or M1-L7 of SEQ ID NO:116. Polynucleotide sequences encoding these polypeptides are also provided. The present invention also encompasses the use of these C-terminal clone 204305 deletion polypeptides as immunogenic and/or antigenic epitopes as described elsewhere herein.

### Features of the Polypeptide Encoded by Gene No:92

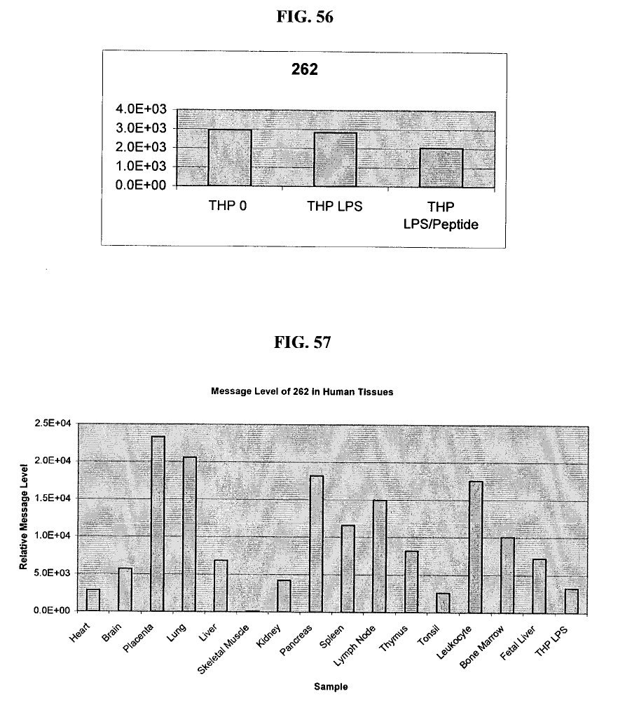

In confirmation that the 262 (SEQ ID NO:92; SEQ ID NO: 262; Table II) polynucleotide and/or its encoded polypeptide are involved in the NF-kB pathway, real-time PCR analyses was used to show that 262 expression is NF-kB-dependent, as shown in FIG. 56. 262 was expressed in unstimulated THP-1 monocytes as a control. In response to stimulation with LPS, steady-state levels of 262 mRNA increased. This increase in expression was inhibited by inclusion of the selective NF-kB inhibitor, BMS-205820.

In an effort to identify additional associations of the 262 polynucleotide and/or its encoded polypeptide with the NF-kB pathway in other human tissues, RT-PCR was performed on a variety of tissues. The results of these experiments indicate that 262 mRNA is expressed at predominately high levels in placenta, lung, pancreas, leukocyte, and to a lesser extent in, lymph node, spleen, bone marrow, thymus, in addition to other tissues as shown (see FIG. 57). The increased expression levels in immune tissues is consistent with the 262 representing a NFkB modulated polynucleotide and polypeptide.

The confirmation that the expression of the 262 polynucleotide and encoded peptide are inhibited by NFkB suggests that antagonists directed against the 262 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 262 polynucleotide and/or encoded peptide are useful for decreasing NF-kB activity, decreasing apoptotic events, and/or increasing IkBa expression or activity levels.

The 262 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 262 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in placenta, in combination with its association with the NFkB pathway suggests the 262 polynucleotides and polypeptides, preferably antagonists, may be useful in treating, diagnosing, prognosing, and/or preventing reproductive and vascular diseases and/or disorders.

The expression of 262 transcripts in lung tissue, in combination with its association with the NFkB pathway suggests the potential utility for 262 polynucleotides and polypeptides, preferably antagonists, in treating, diagnosing, prognosing, and/or preventing pulmonary diseases and disorders which include the following, not limiting examples: ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immumocompromised, HIV, etc.), for example.

Moreover, polynucleotides and polypeptides, including fragments and/or antagonists thereof, have uses which include, directly or indirectly, treating, preventing, diagnosing, and/or prognosing the following, non-limiting, pulmonary infections: pnemonia, bacterial pnemonia, viral pnemonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pnemonia (for example, as caused by Mycobacterium tuberculosis, etc.) mycoplasma pnemonia, fungal pnemonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pnemonia, aspiration pnemonia, Nocordia sp. Infections, parasitic pnemonia (for example, as caused by Strongyloides, Toxoplasma gondii, etc.) necrotizing pnemonia, in addition to any other pulmonary disease and/or disorder (e.g., non-pneumonia) implicated by the causative agents listed above or elsewhere herein.

The expression in pancreas cells, in combination with its association with the NFkB pathway suggests the 262 polynucleotides and polypeptides, preferably antagonists, may be useful in treating, diagnosing, prognosing, and/or preventing pancreatic, in addition to metabolic and gastrointestinal disorders. In preferred embodiments, 262 polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of -cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

The expression in leukocyte, in combination with its association with the NFkB pathway suggests the 262 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:97

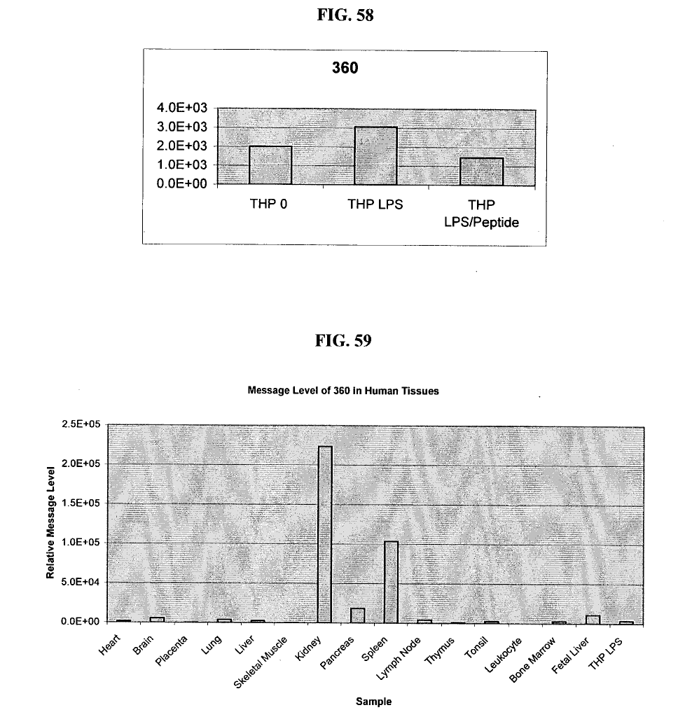

In confirmation that the 360 (SEQ ID NO:97; Table II) polynucleotide and/or its encoded polypeptide are involved in the NF-kB pathway, real-time PCR analyses was used to show that 360 expression is NF-kB-dependent, as shown in FIG. 58. 360 was expressed in unstimulated THP-1 monocytes as a control. In response to stimulation with LPS, steady-state levels of 360 mRNA increased. This increase in expression was inhibited by inclusion of the selective NF-kB inhibitor, BMS-205820.

In an effort to identify additional associations of the 360 polynucleotide and/or its encoded polypeptide with the NF-kB pathway in other human tissues, RT-PCR was performed on a variety of tissues. The results of these experiments indicate that 360 mRNA is expressed at predominately high levels in kidney, spleen, and to a lesser extent in other tissues as shown (see FIG. 59). The increased expression levels in immune tissues is consistent with the 360 representing a NFkB modulated polynucleotide and polypeptide.

The confirmation that the expression of the 360 polynucleotide and encoded peptide are inhibited by NFkB suggests that antagonists directed against the 360 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 360 polynucleotide and/or encoded peptide are useful for decreasing NF-kB activity, decreasing apoptotic events, and/or increasing IkBa expression or activity levels.

The 360 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 360 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in kidney cells, in combination with its association with the NFkB pathway suggests the 360 polynucleotides and polypeptides, preferably antagonists, may be useful in treating, diagnosing, prognosing, and/or preventing renal diseases and/or disorders, which include, but are not limited to: nephritis, renal failure, nephrotic syndrome, urinary tract infection, hematuria, proteinuria, oliguria, polyuria, nocturia, edema, hypertension, electrolyte disorders, sterile pyuria, renal osteodystrophy, large kidneys, renal transport defects, nephrolithiasis, azotemia, anuria, urinary retention, slowing of urinary stream, large prostate, flank tenderness, full bladder sensation after voiding, enuresis, dysuria, bacteriuria, kidney stones, glomerulonephritis, vasculitis, hemolytic uremic syndromes, thrombotic thrombocytopenic purpura, malignant hypertension, casts, tubulointerstitial kidney diseases, renal tubular acidosis, pyelonephritis, hydronephritis, nephrotic syndrome, crush syndrome, and/or renal colic, in addition to Wilm's Tumor Disease, and congenital kidney abnormalities such as horseshoe kidney, polycystic kidney, and Falconi's syndrome for example.

The expression in spleen, in combination with its association with the NFkB pathway suggests the 360 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

### Features of the Polypeptide Encoded by Gene No:101

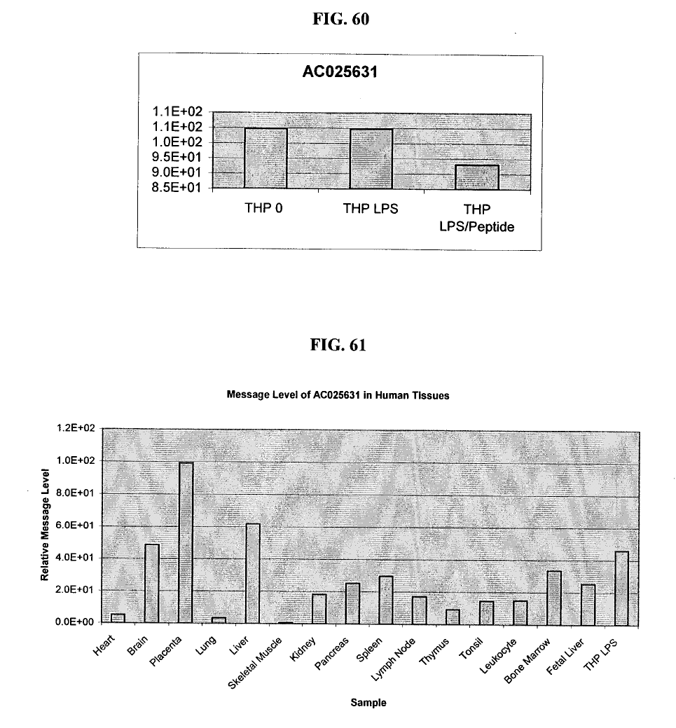

In confirmation that the AC025631 (SEQ ID NO:101; Table II) polynucleotide and/or its encoded polypeptide are involved in the NF-kB pathway, real-time PCR analyses was used to show that AC025631 expression is NF-kB-dependent, as shown in FIG. 60. AC025631 was expressed in unstimulated THP-1 monocytes as a control. In response to stimulation with LPS, steady-state levels of AC025631 mRNA increased. This increase in expression was inhibited by inclusion of the selective NF-kB inhibitor, BMS-205820.

In an effort to identify additional associations of the AC025631 polynucleotide and/or its encoded polypeptide with the NF-kB pathway in other human tissues, RT-PCR was performed on a variety of tissues. The results of these experiments indicate that AC025631 mRNA is expressed at predominately high levels in placenta, liver, brain, and to a lesser extent in other tissues as shown (see FIG. 61).

The confirmation that the expression of the AC025631 polynucleotide and encoded peptide are inhibited by NFkB suggests that antagonists directed against the AC025631 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the AC025631 polynucleotide and/or encoded peptide are useful for decreasing NF-kB activity, decreasing apoptotic events, and/or increasing IkBa expression or activity levels.

The AC025631 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AC025631 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in placenta, in combination with its association with the NFkB pathway suggests the AC025631 polynucleotides and polypeptides, preferably antagonists, may be useful in treating, diagnosing, prognosing, and/or preventing reproductive and vascular diseases and/or disorders.

The expression in liver tissue, in combination with its association with the NFkB pathway suggests the AC025631 polynucleotides and polypeptides, preferably antagonists, may be useful in treating, diagnosing, prognosing, and/or preventing hepatic disorders. Representative uses are described in the “Hyperproliferative Disorders”, “Infectious Disease”, and “Binding Activity” sections below, and elsewhere herein. Briefly, the protein can be used for the detection, treatment, amelioration, and/or prevention of hepatoblastoma, jaundice, hepatitis, liver metabolic diseases and conditions that are attributable to the differentiation of hepatocyte progenitor cells, cirrhosis, hepatic cysts, pyrogenic abscess, amebic abcess, hydatid cyst, cystadenocarcinoma, adenoma, focal nodular hyperplasia, hemangioma, hepatocellulae carcinoma, cholangiocarcinoma, and angiosarcoma, granulomatous liver disease, liver transplantation, hyperbilirubinemia, jaundice, parenchymal liver disease, portal hypertension, hepatobiliary disease, hepatic parenchyma, hepatic fibrosis, anemia, gallstones, cholestasis, carbon tetrachloride toxicity, beryllium toxicity, vinyl chloride toxicity, choledocholithiasis, hepatocellular necrosis, aberrant metabolism of amino acids, aberrant metabolism of carbohydrates, aberrant synthesis proteins, aberrant synthesis of glycoproteins, aberrant degradation of proteins, aberrant degradation of glycoproteins, aberrant metabolism of drugs, aberrant metabolism of hormones, aberrant degradation of drugs, aberrant degradation of drugs, aberrant regulation of lipid metabolism, aberrant regulation of cholesterol metabolism, aberrant glycogenesis, aberrant glycogenolysis, aberrant glycolysis, aberrant gluconeogenesis, hyperglycemia, glucose intolerance, hyperglycemia, decreased hepatic glucose uptake, decreased hepatic glycogen synthesis, hepatic resistance to insulin, portal-systemic glucose shunting, peripheral insulin resistance, hormonal abnormalities, increased levels of systemic glucagon, decreased levels of systemic cortisol, increased levels of systemic insulin, hypoglycemia, decreased gluconeogenesis, decreased hepatic glycogen content, hepatic resistance to glucagon, elevated levels of systemic aromatic amino acids, decreased levels of systemic branched-chain amino acids, hepatic encephalopathy, aberrant hepatic amino acid transamination, aberrant hepatic amino acid oxidative deamination, aberrant ammonia synthesis, aberant albumin secretion, hypoalbuminemia, aberrant cytochromes b5 function, aberrant P450 function, aberrant glutathione S-acyltransferase function, aberrant cholesterol synthesis, and aberrant bile acid synthesis.

Moreover, polynucleotides and polypeptides, including fragments and/or antagonists thereof, have uses which include, directly or indirectly, treating, preventing, diagnosing, and/or prognosing the following, non-limiting, hepatic infections: liver disease caused by sepsis infection, liver disease caused by bacteremia, liver disease caused by Pneomococcal pneumonia infection, liver disease caused by Toxic shock syndrome, liver disease caused by Listeriosis, liver disease caused by Legionnaries' disease, liver disease caused by Brucellosis infection, liver disease caused by Neisseria gonorrhoeae infection, liver disease caused by Yersinia infection, liver disease caused by Salmonellosis, liver disease caused by Nocardiosis, liver disease caused by Spirochete infection, liver disease caused by Treponema pallidum infection, liver disease caused by Brrelia burgdorferi infection, liver disease caused by Leptospirosis, liver disease caused by Coxiella burnetii infection, liver disease caused by Rickettsia richettsii infection, liver disease caused by Chlamydia trachomatis infection, liver disease caused by Chlamydia psittaci infection, liver disease caused by hepatitis virus infection, liver disease caused by Epstein-Barr virus infection in addition to any other hepatic disease and/or disorder implicated by the causative agents listed above or elsewhere herein.

### Features of the Polypeptide Encoded by Gene No: 102

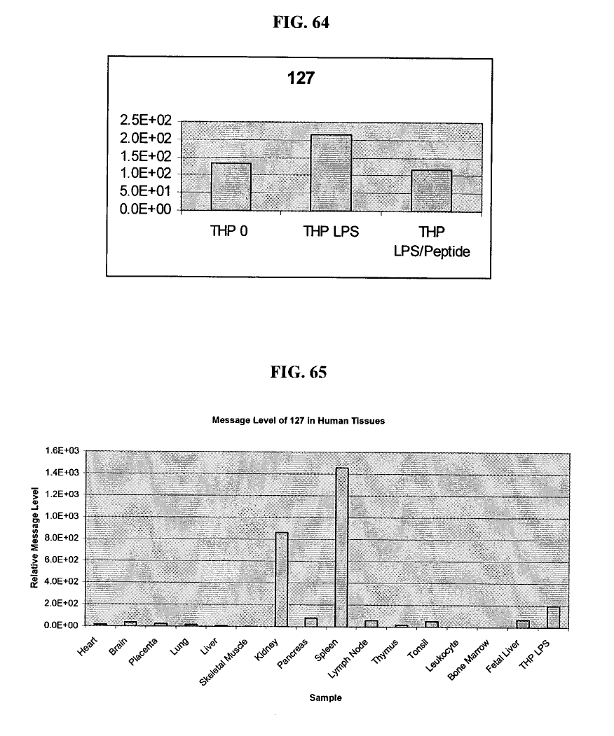

In confirmation that the 127 (SEQ ID NO:101; Table II) polynucleotide and/or its encoded polypeptide are involved in the NF-kB pathway, real-time PCR analyses was used to show that 127 expression is NF-kB-dependent, as shown in FIG. 64. 127 was expressed in unstimulated THP-1 monocytes as a control. In response to stimulation with LPS, steady-state levels of 127 mRNA increased. This increase in expression was inhibited by inclusion of the selective NF-kB inhibitor, BMS-205820.

In an effort to identify additional associations of the 127 polynucleotide and/or its encoded polypeptide with the NF-kB pathway in other human tissues, RT-PCR was performed on a variety of tissues. The results of these experiments indicate that 127 mRNA is expressed at predominately high levels in spleen, kidney, and to a lesser extent in other tissues as shown (see FIG. 65).

The confirmation that the expression of the 127 polynucleotide and encoded peptide are inhibited by NFkB suggests that antagonists directed against the 127 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 127 polynucleotide and/or encoded peptide are useful for decreasing NF-kB activity, decreasing apoptotic events, and/or increasing IkBa expression or activity levels.

The 127 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 127 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

The expression in spleen, in combination with its association with the NFkB pathway suggests the 127 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

The expression in kidney, in combination with its association with the NFkB pathway suggests the 127 polynucleotides and polypeptides, preferably antagonists, may be useful in treating, diagnosing, prognosing, and/or preventing renal diseases and/or disorders, which include, but are not limited to: nephritis, renal failure, nephrotic syndrome, urinary tract infection, hematuria, proteinuria, oliguria, polyuria, nocturia, edema, hypertension, electrolyte disorders, sterile pyuria, renal osteodystrophy, large kidneys, renal transport defects, nephrolithiasis, azotemia, anuria, urinary retention, slowing of urinary stream, large prostate, flank tenderness, full bladder sensation after voiding, enuresis, dysuria, bacteriuria, kidney stones, glomerulonephritis, vasculitis, hemolytic uremic syndromes, thrombotic thrombocytopenic purpura, malignant hypertension, casts, tubulointerstitial kidney diseases, renal tubular acidosis, pyelonephritis, hydronephritis, nephrotic syndrome, crush syndrome, and/or renal colic, in addition to Wilm's Tumor Disease, and congenital kidney abnormalities such as horseshoe kidney, polycystic kidney, and Falconi's syndrome for example.

In preferred embodiments, the following N-terminal clone 127 deletion polypeptides are encompassed by the present invention: M1-V510, E2-V510, L3-V510, K4-V510, K5-V510, S6-V510, P7-V510, D8-V510, G9-V510, G10-V510, W11-V510, G12-V510, W13-V510, V14-V510, I15-V510, V16-V510, F17-V510, V18-V510, S19-V510, F20-V510, L21-V510, M22-V510, P23-V510, F24-V510. I25-V510, A26-V510, Q27-V510, G28-V510, Q29-V510, G30-V510, N31-V510, L32-V510, I33-V510, N34-V510, S35-V510, P36-V510, T37-V510, S38-V510, P39-V510, L40-V510, A41-V510, I42-V510, G43-V510, L44-V510, I45-V510, Y46-V510, I47-V510, L48-V510, K49-V510, K50-V510, E51-V510, V52-V510, E53-V510, H54-V510, H55-V510, Y56-V510, K57-V510, K58-V510, G59-V510, E60-V510, M61-V510, K62-V510, A63-V510, S64-V510, L65-V510, F66-V510, I67-V510, K68-V510, S69-V510, P70-V510, Y71-V510, A72-V510, V73-V510, Q74-V510, N75-V510, I76-V510, R77-V510, K78-V510, T79-V510, A80-V510, A81-V510, V82-V510, G83-V510, V84-V510, L85-V510, Y86-V510, I87-V510, E88-V510, W89-V510, L90-V510, D91-V510, A92-V510, F93-V510, G94-V510, E95-V510, G96-V510, K97-V510, G98-V510, K99-V510, T 100-V510, A101-V510, W102-V510, V103-V510, G104-V510, S105-V510, L106-V510, A107-V510, S108-V510, G109-V510, V110-V510, G111-V510, L112-V510, L113-V510, A114-V510, S115-V510, L116-V510, G117-V510, C118-V510, G119-V510, L120-V510, L121-V510, Y122-V510, T123-V510, A124-V510, T125-V510, V126-V510, T127-V510, I128-V510, T129-V510, C130-V510, Q131-V510, Y132-V510, F133-V510, D134-V510, D135-V510, R136-V510, R137-V510, G138-V510, L139-V510, A140-V510, L141-V510, G142-V510, L143-V510, I144-V510, S145-V510, T146-V510, G147-V510, S148-V510, S149-V510, V150-V510, G151-V510, L152-V510, F153-V50, I154-V510, Y155-V510, A156-V510, A157-V510, L158-V510, Q159-V510, R160-V510, M161-V510, L162-V510, V163-V510, E164-V510, F165-V510, Y166-V510, G167-V510, L168-V510, D169-V510, G170-V510, C171-V510, L172-V510, L173-V510, I174-V510, V175-V510, G176-V510, A177-V510, L178-V510, A179-V510, L180-V510, N181-V510, I182-V510, L183-V510, A184-V510, C185-V510, G186-V510, S187-V510, L188-V510, M189-V510, R190-V510, P191-V510, L192-V510, Q193-V510, S194-V510, S195-V510, D196-V510, C197-V510, P198-V510, L199-V510, P200-V510, K201-V510, K202-V510, I203-V510, A204-V510, P205-V510, E206-V510, D207-V510, L208-V510, P209-V510, D210-V510, K211-V510, Y212-V510, S213-V510, I214-V510, Y215-V510, N216-V510, E217-V510, K218-V510, G219-V510, K220-V510, N221-V510, L222-V510, E223-V510, E224-V510, N225-V510, I226-V510, N227-V510, I228-V510, L229-V510, D230-V510, K231-V510, S232-V510, Y233-V510, S234-V510, S235-V510, E236-V510, E237-V510, K238-V510, C239-V510, R240-V510, I241-V510, T242-V510, L243-V510, A244-V510, N245-V510, G246-V510, D247-V510, W248-V510, K249-V510, Q250-V510, D251-V510, S252-V510, L253-V510, L254-V510, H255-V510, K256-V510, N257-V510, P258-V510, T259-V510, V260-V510, T261-V510, H262-V510, T263-V510, K264-V510, E265-V510, P266-V510, E267-V510, T268-V510, Y269-V510, K270-V510, K271-V510, K272-V510, V273-V510, A274-V510, E275-V510, Q276-V510, T277-V510, Y278-V510, F279-V510, C280-V510, K281-V510, Q282-V510, L283-V510, A284-V510, K285-V510, R286-V510, K287-V510, W288-V510, Q289-V510, L290-V510, Y291-V510, K292-V510, N293-V510, Y294-V510, C295-V510, G296-V510, E297-V510, T298-V510, V299-V510, A300-V510, L301-V510, F302-V510, K303-V510, N304-V510, K305-V510, V306-V510, F307-V510, S308-V510, A309-V510, L310-V510, F311-V510, I312-V510, A313-V510, I314-V510, L315-V510, L316-V510, F317-V510, D318-V510, I319-V510, G320-V510, G321-V510, F322-V510, P323-V510, P324-V510, S325-V510, L326-V510, L327-V510, M328-V510, E329-V510, D330-V510, V331-V510, A332-V510, R333-V510, S334-V510, S335-V510, N336-V510, V337-V510, K338-V510, E339-V510, E340-V510, E341-V510, F342-V510, I343-V510, M344-V510, P345-V510, L346-V510, I347-V510, S348-V510, I349-V510, I350-V510, G351-V510, I352-V510, M353-V510, T354-V510, A355-V510, V356-V510, G357-V510, K358-V510, L359-V510, L360-V510, L361-V510, G362-V510, I363-V510, L364-V510, A365-V510, D366-V510, F367-V510, K368-V510, W369-V510, I370-V510, N371-V510, T372-V510, L373-V510, Y374-V510, L375-V510, Y376-V510, V377-V510, A378-V510, T379-V510, L380-V510, I381-V510, I382-V510, M383-V510, G384-V510, L385-V510, A386-V510, L387-V510, C388-V510, A389-V510, I390-V510, P391-V510, F392-V510, A393-V510, K394-V510, S395-V510, Y396-V510, V397-V510, T398-V510, L399-V510, A400-V510, L401-V510, L402-V510, S403-V510, G404-V510, I405-V510, L406-V510, G407-V510, F408-V510, L409-V510, T410-V510, G411-V510, N412-V510, W413-V510, S414-V510, I415-V510, F416-V510, P417-V510, Y418-V510, V419-V510, T420-V510, T421-V510, K422-V510, T423-V510, V424-V510, G425-V510, I426-V510, E427-V510, K428-V510, L429-V510, A430-V510, H431-V510, A432-V510, Y433-V510, G434-V510, I435-V510, L436-V510, M437-V510, F438-V510, F439-V510, A440-V510, G441-V510, L442-V510, G443-V510, N444-V510, S445-V510, L446-V510, G447-V510, P448-V510, P449-V510, I450-V510, V451-V510, G452-V510, W453-V510, F454-V510, Y455-V510, D456-V510, W457-V510, T458-V510, Q459-V510, T460-V510, Y461-V510, D462-V510, I463-V510, A464-V510, F465-V510, Y466-V510, F467-V510, S468-V510, G469-V510, F470-V510, C471-V510, V472-V510, L473-V510, L474-V510, G475-V510, G476-V510, F477-V510, I478-V510, L479-V510, L480-V510, L481-V510, A482-V510, A483-V510, L484-V510, P485-V510, S486-V510, W487-V510, D488-V510, T489-V510, C490-V510, N491-V510, K492-V510, Q493-V510, L494-V510, P495-V510, K496-V510, P497-V510, A498-V510, P499-V510, T500-V510, T501-V510, F502-V510, L503-V510, and/or Y504-V510 of SEQ ID NO:118. Polynucleotide sequences encoding these polypeptides are also provided. The present invention also encompasses the use of these N-terminal clone 127 deletion polypeptides as immunogenic and/or antigenic epitopes as described elsewhere herein.

In preferred embodiments, the following C-terminal clone 127 deletion polypeptides are encompassed by the present invention: M1-V510, M1-N509, M1-S508, M1-A507, M1-V506, M1-K505, M1-Y504, M1-L503, M1-F502, M1-T501, M1-T500, M1-P499, M1-A498, M1-P497, M1-K496, M1-P495, M1-L494, M1-Q493, M1-K492, M1-N491, M1-C490, M1-T489, M1-D488, M1-W487, M1-S486, M1-P485, M1-L484, M1-A483, M1-A482, M1-L481, M1-L480, M1-L479, M1-I478, M1-F477, M1-G476, M1-G475, M1-L474, M1-L473, M1-V472, M1-C471, M1-F470, M1-G469, M1-S468, M1-F467, M1-Y466, M1-F465, M1-A464, M1-I463, M1-D462, M1-Y461, M1-T460, M1-Q459, M1-T458, M1-W457, M1-D456, M1-Y455, M1-F454, M1-W453, M1-G452, M1-V451, M1-I450, M1-P449, M1-P448, M1-G447, M1-L446, M1-S445, M1-N444, M1-G443, M1-L442, M1-G441, M1-A440, M1-F439, M1-F438, M1-M437, M1-L436, M1-I435, M1-G434, M1-Y433, M1A432, M1-H431, M1-A430, M1-L429, M1-K428, M1-E427, M1I-426, M1-G425, M1-V424, M1-T423, M1-K422, M1-T421, M1-T420, M1-V419, M1-Y418, M1-P417, M1-F416, M1-I415, M1-S414, M1-W413, M1-N412, M1-G411, M1-T410, M1-L409, M1-F408, M1-G407, M1-L406, M1-I405, M1-G404, M1-S403, M1-L402, M1-L401, M1-A400, M1-L399, M1-T398, M1-V397, M1-Y396, M1-S395, M1-K394, M1-A393, M1-F392, M1-P391, M1-I390, M1-A389, M1-C388, M1-L387, M1-A386, M1-L385, M1-G384, M1-M383, M1-I382, M1-I381, M1-L380, M1-T379, M1-A378, M1-V377, M1-Y376, M1-L375, M1-Y374, M1-L373, M1-T372, M1-N371, M1-I370, M1-W369, M1-K368, M1-F367, M1-D366, M1-A365, M1-L364, M1-I363, M1-G362, M1-L361, M1-L360, M1-L359, M1-K358, M1-G357, M1-V356, M1-A355, M1-T354, M1-M353, M1-I352, M1-G351, M1-I350, M1-I349, M1-S348, M1-I347, M1-L346, M1-P345, M1-M344, M1-I343, M1-F342, M1-E341, M1-E340, M1-E339, M1-K338, M1-V337, M1-N336, M1-S335, M1-S334, M1-R333, M1-A332, M1-V331, M1-D330, M1-E329, M1-M328, M1-L327, M1-L326, M1-S325, M1-P324, M1-P323, M1-F322, M1-G321, M1-G320, M1-I319, M1-D318, M1-F317, M1-L316, M1-L315, M1-I314, M1-A313, M1-I312, M1-F311, M1-L310, M1-A309, M1-S308, M1-F307, M1-V306, M1-K305, M1-N304, M1-K303, M1-F302, M1-L301, M1-A300, M1-V299, M1-T298, M1-E297, M1-G296, M1-C295, M1-Y294, M1-N293, M1-K292, M1-Y291, M1-L290, M1-Q289, M1-W288, M1-K287, M1-R286, M1-K285, M1-A284, M1-L283, M1-Q282, M1-K281, M1-C280, M1-F279, M1-Y278, M1-T277, M1-Q276, M1-E275, M1-A274, M1-V273, M1-K272, M1-K271, M1-K270, M1-Y269, M1-T268, M1-E267, M1-P266, M1-E265, M1-K264, M1-T263, M1-H262, M1-T261, M1-V260, M1-T259, M1-P258, M1-N257, M1-K256, M1-H255, M1-L254, M1-L253, M1-S252, M1-D251, M1-Q250, M1-K249, M1-W248, M1-D247, M1-G246, M1-N245, M1-A244, M1-L243, M1-T242, M1-I241, M1-R240, M1-C239, M1-K238, M1-E237, M1-E236, M1-S235, M1-S234, M1-Y233, M1-S232, M1-K231, M1-D230, M1-L229, M1-I228, M1-N227, M1-I226, M1-N225, M1-E224, M1-E223, M1-L222, M1-N221, M1-K220, M1-G219, M1-K218, M1-E217, M1-N216, M1-Y215, M1-I214, M1-S213, M1-Y212, M1-K211, M1-D210, M1-P209, M1-L208, M1-D207, M 1-E206, M1-P205, M1-A204, M1-I203, M1-K202 M1-K201, M1-P200, M1-L199, M1-P198, M1-C197, M1-D196, M1-S195, M1-S194, M1-Q193, M1-L192, M1-P191, M1-R190, M1-M189, M1-L188, M1-S187, M1-G186, M1-C185, M1-A184, M1-L183, M1-I182, M1-N181, M1-L180, M1-A179, M1-L178, M1-A177, M1-G176, M1-V175, M1-I174, M1-L173, M1-L172, M1-C171, M1-G170, M1-D169, M1-L168, M1-G167, M1-Y166, M1-F165, M1-E164, M1-V163, M1-L162, M1-M161, M1-R160, M1-Q159, M1-L158, M1-A157, M1-A156, M1-Y155, M1-I154, M1-F153, M1-L152, M1-G151, M1-V150, M1-S149, M1-S148, M1-G147, M1-T146, M1-S145, M1-I144, M1-L143, M1-G142, M1-L141, M1-A140, M1-L139, M1-G138, M1-R137, M1-R136, M1-D135, M1-D134, M1-F133, M1-Y132, M1-Q131, M1-C130, M1-T129, M1-I128, M1-T127, M1-V126, M1-T125, M1-A124, M1-T123, M1-Y122, M1-L121, M1-L120, M1-G119, M1-C118, M1-G117, M1-L116, M1-S115, M1-A114, M1-L113, M1-L112, M1-G111, M1-V110, M1-G109, M1-S108, M1-A107, M1-L106, M1-S105, M1-G104, M1-V103, M1-W102, M1-A101, M1-T100, M1-K99, M1-G98, M1-K97, M1-G96, M1-E95, M1-G94, M1-F93, M1-A92, M1-D91, M1-L90, M1-W89, M1-E88, M1-I87, M1-Y86, M1-L85, M1-V84, M1-G83, M1-V82, M1-A81, M1-A80, M1-T79, M1-K78, M1-R77, M1-I76, M1-N75, M1-Q74, M1-V73, M1-A72, M1-Y71, M1-P70, M1-S69, M1-K68, M1-167, M1-F66, M1-L65, M1-S64, M1-A63, M1-K62, M1-M61, M1-E60, M1-G59, M1-K58, M1-K57, M1-Y56, M1-H55, M1-H54, M1-E53, M1-V52, M1-E51, M1-K50, M1-K49, M1-L48, M1-I47, M1-Y46, M1-I45, M1-L44, M1-G43, M1-I42, M1-A41, M1-L40, M1-P39, M1-S38, M1-T37, M1-P36, M1-S35, M1-N34, M1-I33, M1-L32, M1-N31, M1-G30, M1-Q29, M1-G28, M1-Q27, M1-A26, M1-I25, M1-F24, M1-P23, M1-M22, M1-L21, M1-F20, M1-S19, M1-V18, M1-F17, M1-V16, M1-I15, M1-V14, M1-W13, M1-G12, M1-W11, M1-G10, M1-G9, M1-D8, and/or M1-P7 of SEQ ID NO: 118. Polynucleotide sequences encoding these polypeptides are also provided. The present invention also encompasses the use of these C-terminal clone 127 deletion polypeptides as immunogenic and/or antigenic epitopes as described elsewhere herein.

### Features of the Polypeptide Encoded by Gene No:103

In confirmation that the 36d5 (SEQ ID NO:103; SEQ ID NO: 283; Table IV) polynucleotide and/or its encoded polypeptide are involved in the NF-kB pathway, real-time PCR analyses was used to show that 36d5 expression is NF-kB-dependent, as shown in FIG. 79. 36d5 was expressed in unstimulated THP-1 monocytes as a control. In response to stimulation with LPS, steady-state levels of 36d5 mRNA increased. This increase in expression was inhibited by inclusion of the selective NF-kB inhibitor, BMS-205820, in addition to LPS/dexamethasone treatment.

The confirmation that the expression of the 36d5 polynucleotide and encoded peptide are inhibited by NFkB suggests that antagonists directed against the 36d5 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 36d5 polynucleotide and/or encoded peptide are useful for decreasing NF-kB activity, decreasing apoptotic events, and/or increasing IkBa expression or activity levels.

### Features of the Polypeptide Encoded by Gene No:104

In confirmation that the 37e4 (SEQ ID NO:104; Table IV) polynucleotide and/or its encoded polypeptide are involved in the NF-kB pathway, real-time PCR analyses was used to show that 37e4 expression is NF-kB-dependent, as shown in FIG. 79. 37e4 was expressed in unstimulated THP-1 monocytes as a control. In response to stimulation with LPS, steady-state levels of 37e4 mRNA increased. This increase in expression was inhibited by inclusion of the selective NF-kB inhibitor, BMS-205820, in addition to LPS/dexamethasone treatment.

The confirmation that the expression of the 37e4 polynucleotide and encoded peptide are inhibited by NFkB suggests that antagonists directed against the 37e4 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 37e4 polynucleotide and/or encoded peptide are useful for decreasing NF-kB activity, decreasing apoptotic events, and/or increasing IkBa expression or activity levels.

The 37E4 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 37E4 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

### Features of the Polypeptide Encoded by Gene No:106

In confirmation that the 42e7 (SEQ ID NO: 106; Table IV) polynucleotide and/or its encoded polypeptide are involved in the NF-kB pathway, real-time PCR analyses was used to show that 42e7 expression is NF-kB-dependent, as shown in FIG. 79. 42e7 was expressed in unstimulated THP-1 monocytes as a control. In response to stimulation with LPS, steady-state levels of 42e7 mRNA increased. This increase in expression was inhibited by inclusion of the selective NF-kB inhibitor, BMS-205820, in addition to LPS/dexamethasone treatment.

The confirmation that the expression of the 42e7 polynucleotide and encoded peptide are inhibited by NFkB suggests that antagonists directed against the 42e7 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 42e7 polynucleotide and/or encoded peptide are useful for decreasing NF-kB activity, decreasing apoptotic events, and/or increasing IkBa expression or activity levels.

The 42E7 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 42E7 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

### Features of the Polypeptide Encoded by Gene No:107

In confirmation that the 105b2 (SEQ ID NO: 107; Table IV) polynucleotide and/or its encoded polypeptide are involved in the NF-kB pathway, real-time PCR analyses was used to show that 105b2 expression is NF-kB-dependent, as shown in FIG. 79. 105b2 was expressed in unstimulated THP-1 monocytes as a control. In response to stimulation with LPS, steady-state levels of 105b2 mRNA increased. This increase in expression was inhibited by inclusion of the selective NF-kB inhibitor, BMS-205820, in addition to LPS/dexamethasone treatment.

The confirmation that the expression of the 105b2 polynucleotide and encoded peptide are inhibited by NFkB suggests that antagonists directed against the 105b2 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 105b2 polynucleotide and/or encoded peptide are useful for decreasing NF-kB activity, decreasing apoptotic events, and/or increasing IkBa expression or activity levels.

The 105B2 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 105B2 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

### Features of the Polypeptide Encoded by Gene No:108

In confirmation that the 41h1 (SEQ ID NO:108; Table IV) polynucleotide and/or its encoded polypeptide are involved in the NF-kB pathway, real-time PCR analyses was used to show that 41h1 expression is NF-kB-dependent, as shown in FIG. 79. 41h1 was expressed in unstimulated THP-1 monocytes as a control. In response to stimulation with LPS, steady-state levels of 41h1 mRNA increased. This increase in expression was inhibited by inclusion of the selective NF-kB inhibitor, BMS-205820, in addition to LPS/dexamethasone treatment.

The confirmation that the expression of the 41h1 polynucleotide and encoded peptide are inhibited by NFkB suggests that antagonists directed against the 41h1 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the 41h1 polynucleotide and/or encoded peptide are useful for decreasing NF-kB activity, decreasing apoptotic events, and/or increasing IkBa expression or activity levels.

The 41H1 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The 41H1 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

### Features of the Polypeptide Encoded by Gene No:109

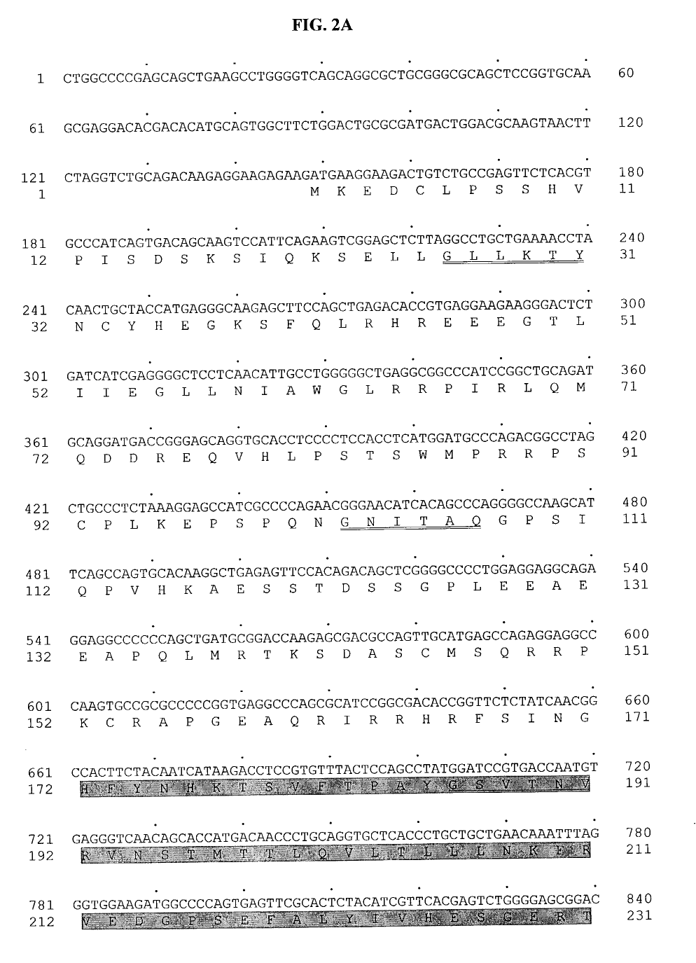

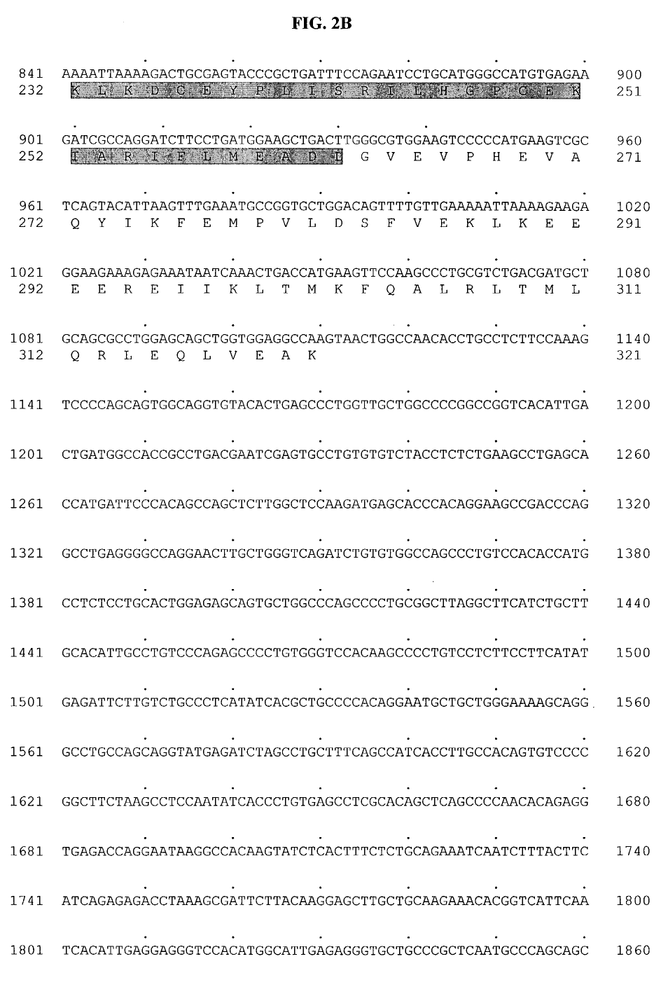

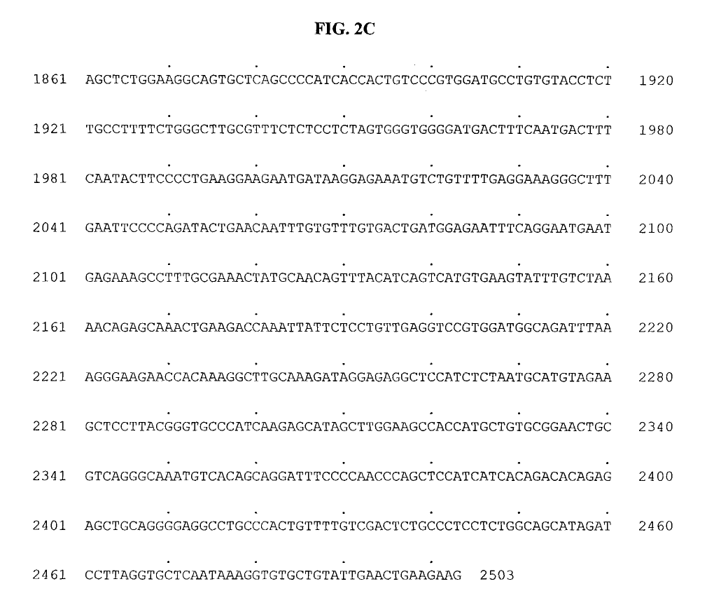

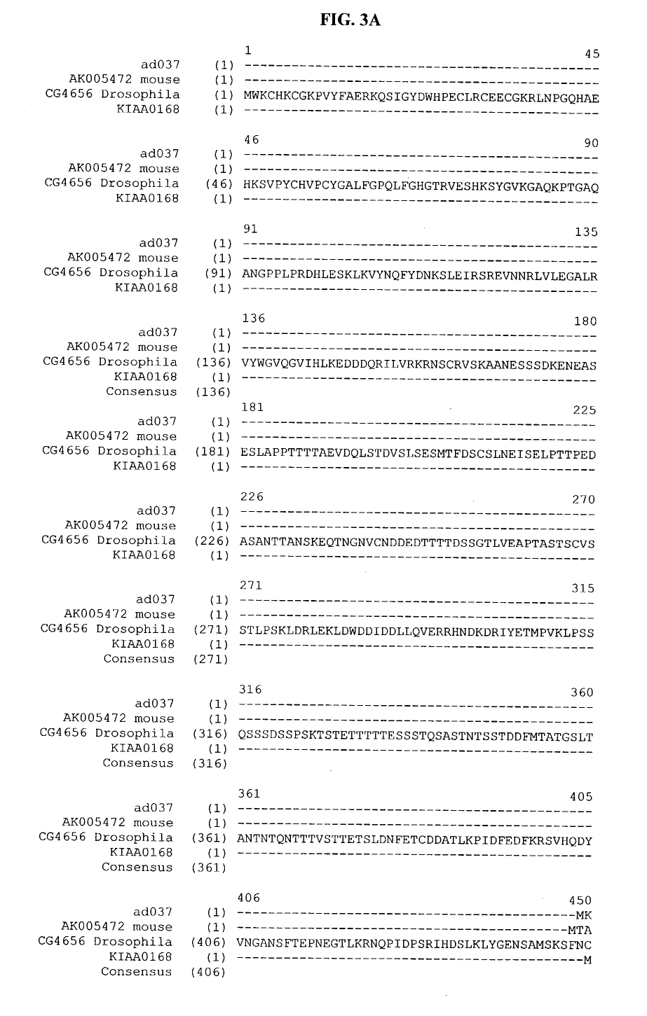

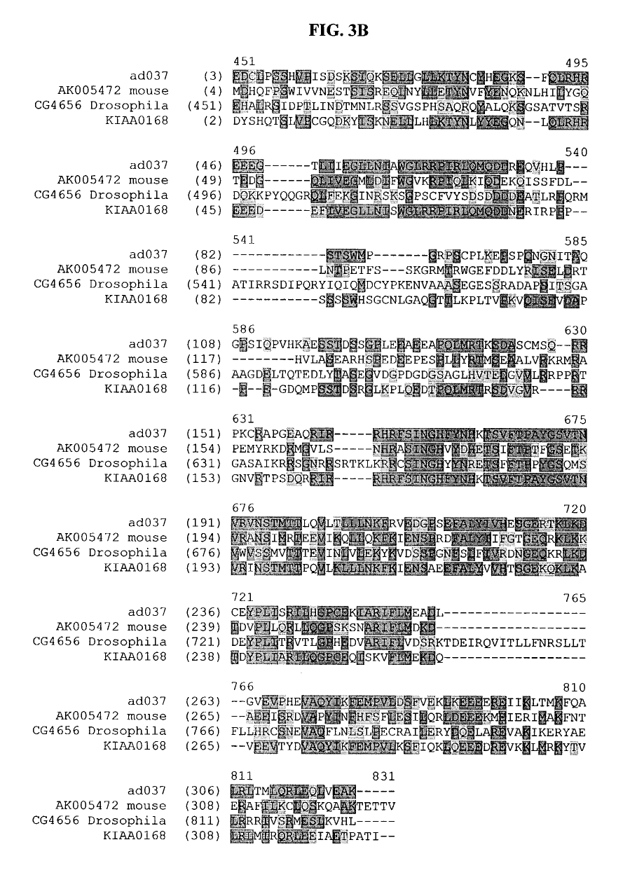

The polypeptide of this gene provided as SEQ ID NO:125 (FIGS. 2A-C), encoded by the polynucleotide sequence according to SEQ ID NO:126 (FIGS. 2A-C), has significant homology at the nucleotide and amino acid level to the hypothetical protein KIAA0168, also referred to as the Ras association RalGDS/AF-6 domain family 2 protein (KIAA0168; Genbank Accession No. gil13274205; SEQ ID NO:129), the hypothetical mouse protein AK005472 (AK005472; Genbank Accession No. gil12838052; SEQ ID NO:130), and the Drosophila protein CG4656 (CG4656; Genbank Accession No. gil7300961; SEQ ID NO:131). An alignment of the AD037 polypeptide with these proteins is provided in FIGS. 3A-B.

The determined nucleotide sequence of the AD037 cDNA in FIGS. 2A-C (SEQ ID NO:125) contains an open reading frame encoding a protein of about 321 amino acid residues, with a deduced molecular weight of about 36.7 kDa. The amino acid sequence of the predicted AD037 polypeptide is shown in FIGS. 2A-C (SEQ ID NO:126). The AD037 protein shown in FIGS. 2A-C was determined to share significant identity and similarity to several proteins. Specifically, the AD037 protein shown in FIGS. 2A-C was determined to be about 59% identical and 67% similar to the hypothetical protein KIAA0168, also referred to as the Ras association RaIGDS/AF-6 domain family 2 protein (KIAA0168; Genbank Accession No. gil13274205; SEQ ID NO:129), to be about 38% identical and 52% similar to the hypothetical mouse protein AK005472 (AK005472; Genbank Accession No. gil12838052; SEQ ID NO:130), and to be about 31% identical and 42% similar to the Drosophila protein CG4656 (CG4656; Genbank Accession No. gil7300961; SEQ ID NO:131).

Analysis of the AD037 polypeptide determined that it contains a Ras association motif which is a domain shared by members of the RasGTP effectors family located at about amino acid 172 to about amino acid 262 of SEQ ID NO:126. The presence of this domain is consistent with the shared identity with the human Ras association RalGDS/AF-6 protein.

In preferred embodiments, the following Ras association motif polypeptide is encompassed by the present invention: HFYNHKTSVFTPAYGSVTNVRVNSTMTTLQVLTLLLNKFRVEDGPSEFALYIV HESGERTKLKDCEYPLISRILHGPCEKIARIFLMEADL (SEQ ID NO:141). Polynucleotides encoding this polypeptide are also provided. The present invention also encompasses the use of this AD037 Ras association motif polypeptide as an immunogenic and/or antigenic epitope as described elsewhere herein.

The present invention encompassess the coding region of the AD037 polynucleotide. Specifically, the present invention encompasses the polynucleotide corresponding to nucleotides 149 thru 1121 of SEQ ID NO:125, and the polypeptide corresponding to amino acids 2 thru 321 of SEQ ID NO:126. Also encompassed are recombinant vectors comprising said encoding sequence, and host cells comprising said vector.

In preferred embodiments, the present invention encompasses a polynucleotide lacking the initiating start codon, in addition to, the resulting encoded polypeptide of AD037. Specifically, the present invention encompasses the polynucleotide corresponding to nucleotides 152 thru 1121 of SEQ ID NO:125, and the polypeptide corresponding to amino acids 2 thru 321 of SEQ ID NO:126. Also encompassed are recombinant vectors comprising said encoding sequence, and host cells comprising said vector.

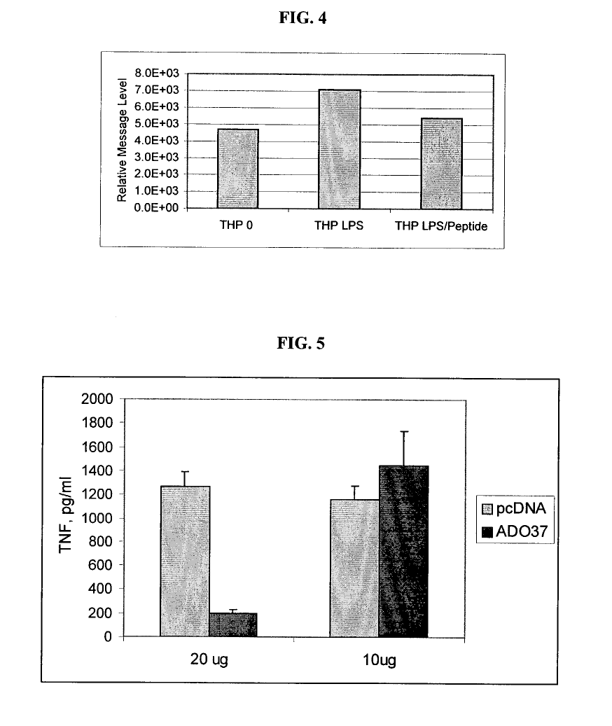

In confirmation that the AD037 polypeptide is involved in the NF-kB pathway, real-time PCR analyses was used to show that AD037 expression is NF-kB-dependent, as shown in FIG. 4. AD037 was expressed in unstimulated THP-1 monocytes as a control. In response to stimulation with LPS, steady-state levels of AD037 mRNA increased. This increase in expression was inhibited by inclusion of the selective NF-kB inhibitor, BMS-205820. When AD037 was overexpressed in THP-1 monocytes, AD037 significantly inhibited TNFα secretion, suggesting that it plays a role in this NF-kB-dependent response, as shown in FIG. 5.

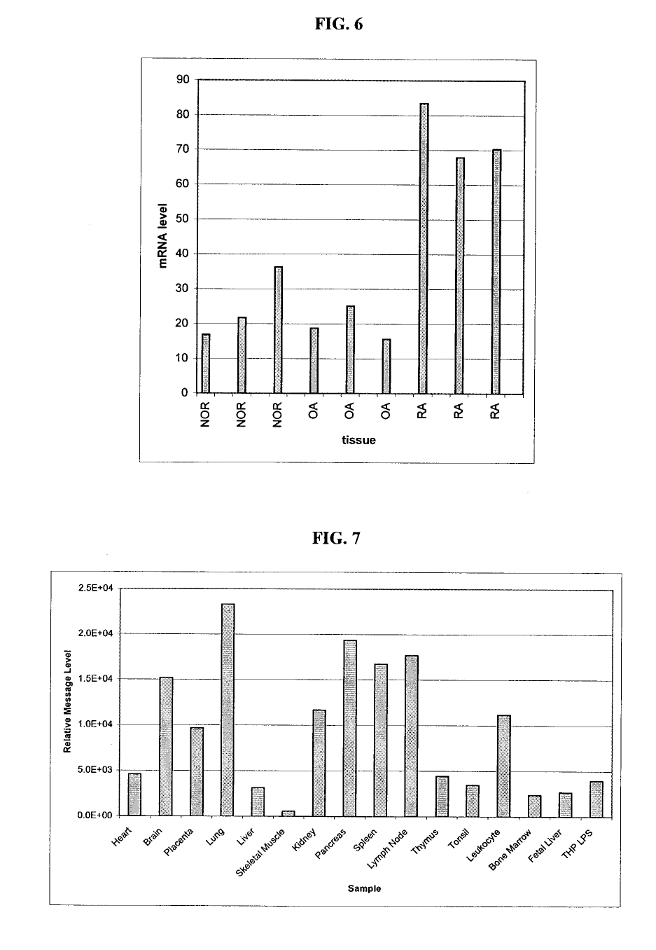

Additional real-time PCR experiments have provided additional evidence that AD037 is involved in the NF-kB pathway. Specifically, it has been discovered that expression of AD037 mRNA was elevated in synovial samples derived from rheumatoid arthritis patients as compared to osteoarthritis synovium, and synovium derived from joint trauma controls (see FIG. 6).

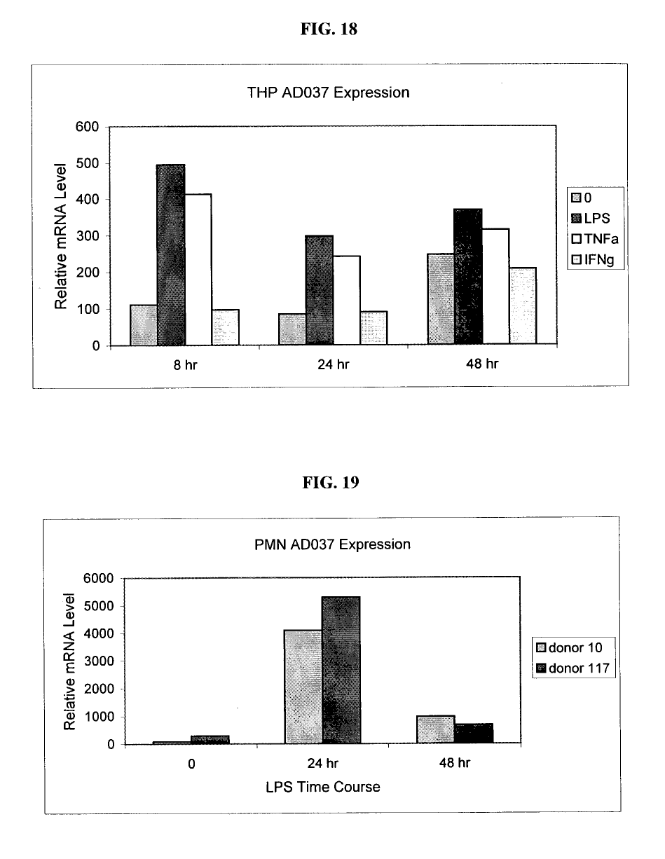

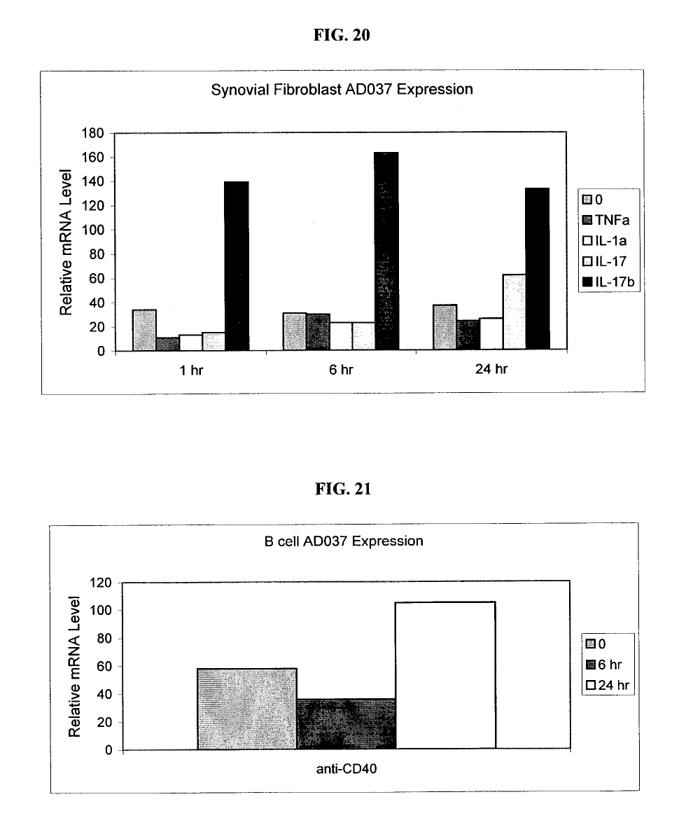

In further confirmation of the association of AD037 with the NF-kB pathway, AD037 mRNA was elevated in various human primary cell lines in response to NF-kB stimuli. Specifically, AD037 mRNA was upregulated in THP-1 cells in response to LPS and TNFα stimuli, as shown in FIG. 18. Consistent with the role of AD037 in NF-kB, little upregulation was observed in response to IFN-γ, which fails to activate the NF-kB pathway. As shown in FIG. 19, AD037 mRNA was strongly upregulated in human peripheral blood neutrophils in response to LPS stimulation. As shown in FIG. 20, AD037 mRNA was selectively upregulated in synovial fibroblasts in response to stimulation with an IL-17B-Ig fusion protein. No upregulation was observed in response to IL-1α, TNF-α, or IL-17. As shown in FIG. 21, AD037 mRNA was induced in human peripheral blood B cells in response to CD40 crosslinking, another pathway known to activate NF-kB.

In an effort to identify additional associations with the NF-kB pathway in other human tissues, RT-PCR was performed on a variety of tissues. The results of these experiments indicate that AD037 mRNA is expressed at predominately high levels in hematopoietic tissues including lymph node, spleen and leukocytes. High levels of expression were also detected in non-hematopoietic tissues including lung, pancreas, brain, kidney, and placenta. Lower levels of expression were detected in heart, liver, thymus, tonsil, bone marrow, fetal liver, and skeletal muscle (see FIG. 7). The increased expression levels in immune tissues is consistent with the AD037 representing a NFkB modulated polynucleotide and polypeptide.

The predominate expression in lymph node, spleen and leukocytes tissue, in combination with its association with the NFkB pathway suggests the AD037 polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

Since many proteins involved in the NF-kB pathway, and signalling proteins, in general are cell surface proteins and/or receptors, experiments were performed to assess where AD037 localizes in the cell. The full length AD037 sequence was cloned into a Flag-tagged expression vector which was transfected into Cos7 cells. To determine if the protein was expressed, lysates from Cos transfectants were electrophoresed and blotted with anti-Flag antibodies (see FIG. 8). A specific band of the expected size (approximately 40 kD) was detected in cells transfected with AD037 relative to cells transfected with vector alone.

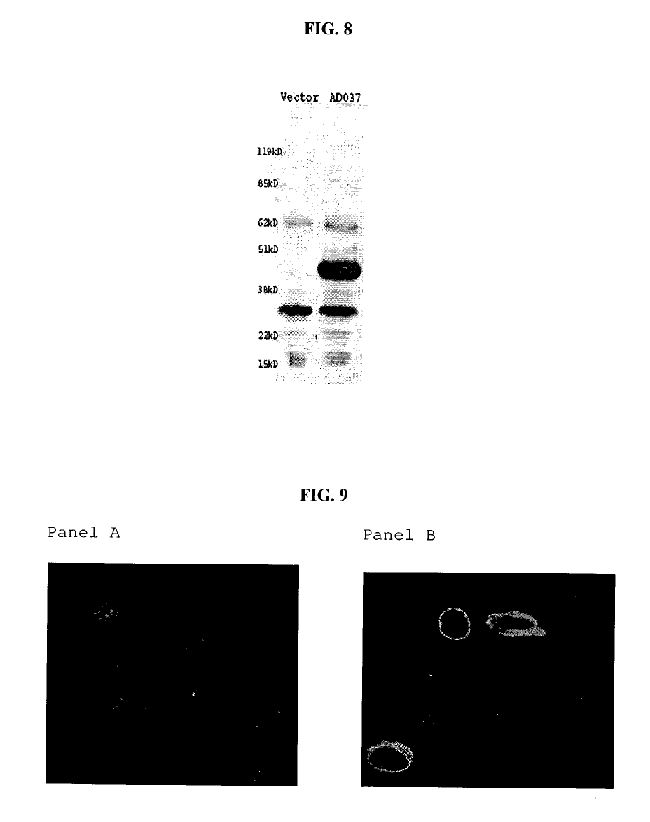

In order to localize AD037 in cells, Cos transfectants were stained with anti-Flag antibodies, detected with FITC-labeled secondary antibodies, and analyzed by confocal microscopy (see FIG. 9). Specific fluorescence was detected in cells transfected with AD037, but not in cells transfected with vector alone. The expressed AD037 localized to the plasma membrane in the transfectants. Since AD037 lacks a transmembrane domain, this suggests that it associate with a membrane-localized protein.

Moreover, in an effort to further confirm the NF-κB-dependent expression of AD037, two approaches were investigated. In the first approach, additional inhibitors of the NF-κB pathway were used to assess their affect on AD037 expression. Although it has other transcriptional effects, dexamethasone inhibits NF-κB activity via glucocorticoid receptor-mediated transrepression (Reichardt et al. (2001) EMBO J. 20:7168-7173). The compound 4(2′-aminoethyl)amino-1,8-dimethylimidazo(1,2-a) quinoxaline, is a selective IKK-2 inhibitor (International Publication No. WO 02/60386, Published Oct. 10, 2002; Burke et al. (2003) J. Biol. Chem. 278:1450-1456).

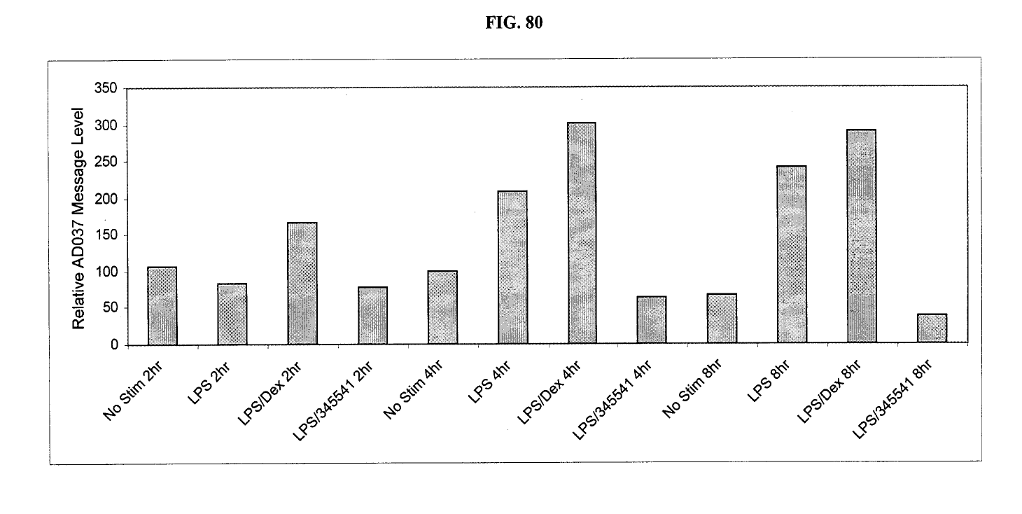

As shown in FIG. 80, LPS-mediated induction of AD037 mRNA peaked between 4 and 8 hours post stimulation. At these time points, addition of the 4(2′-aminoethyl)amino-1,8-dimethylimidazo(1,2-a) quinoxaline compound significantly inhibited AD037 expression. In contrast, addition of dexamethasone failed to inhibit expression. Since dexamethasone is a glucocorticoid receptor agonist, the AD037 promoter may contain a glucocorticoid response element that overrides the effects of transrepression (Hoffman et al. (2002) Biol. Chem. 383:1947-1951).

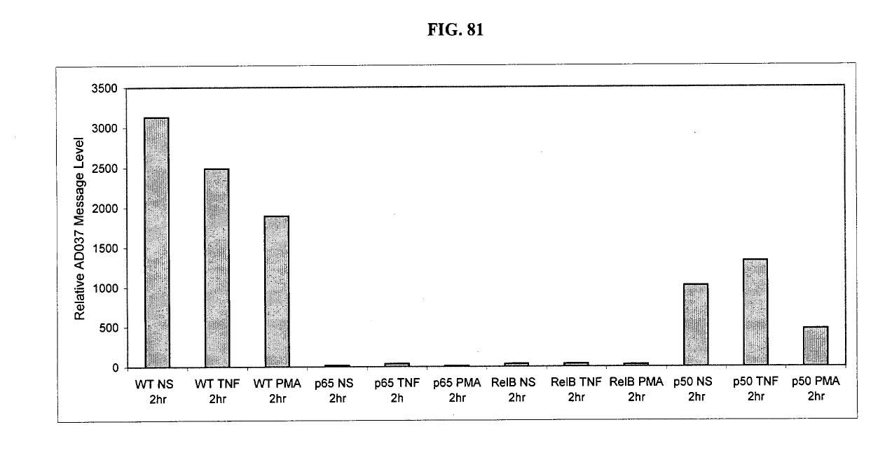

In the second approach, the expression of AD037 was profiled in mouse embryonic fibroblasts derived from germline knockouts of different NF-κB family members. Wild type 3T3 cells, and embryonic fibroblasts derived from germline knockouts of p65, RelB, and p50 were stimulated for 2 hours with either TNFα or PMA. At each time point, mRNA was isolated and real time PCR was performed. Expression of the mouse homologue of AD037 was constitutive in wild type fibroblasts (see FIG. 81). In contrast, no expression was detected in fibroblasts derived from either p65 or RelB deficient fibroblasts. Reduced levels of AD037 were detected in fibroblasts derived from p50 knockouts. The data suggests that complexes containing p65, RelB, and p50 are required for AD037 expression.

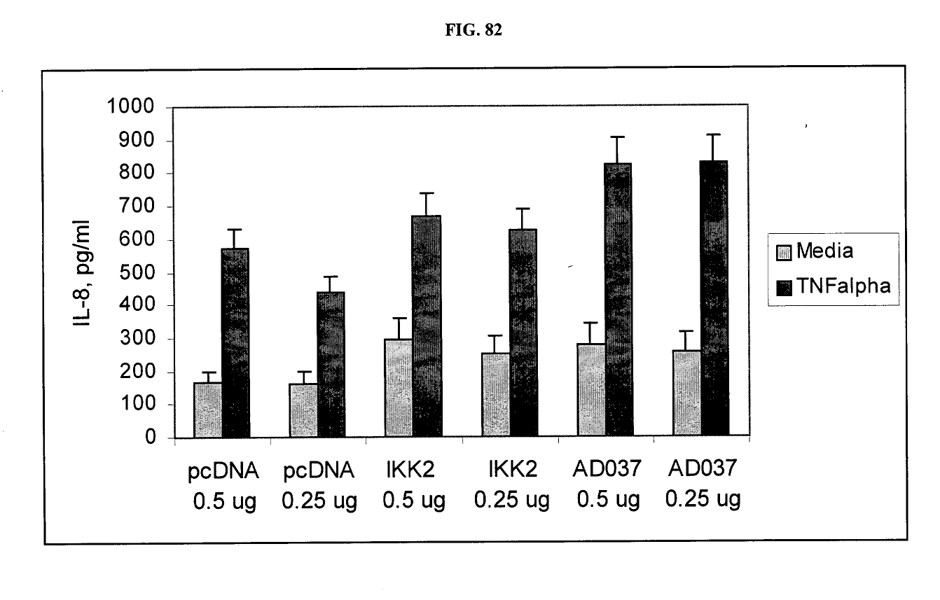

To further characterize the function of AD037, H292 epithelial cells were transfected with expression constructs encoding either wild type IKK2 or wild type AD037. The transfectants were stimulated with TNFα to induce IL-8, a response dependent on NF-κB activity (Hoffmann et al. (2002) J. Leukoc. Biol. 72:847-855). As expected, transfection of wild type IKK2 significantly increased both basal and induced levels of IL-8 as compared to transfection with vector alone (see FIG. 82). Transfection of wild type AD037 also increased both basal and induced levels of IL-8 above that stimulated by vector, or by IKK2. The data suggests that AD037 can functionally interact with the NF-κB pathway.

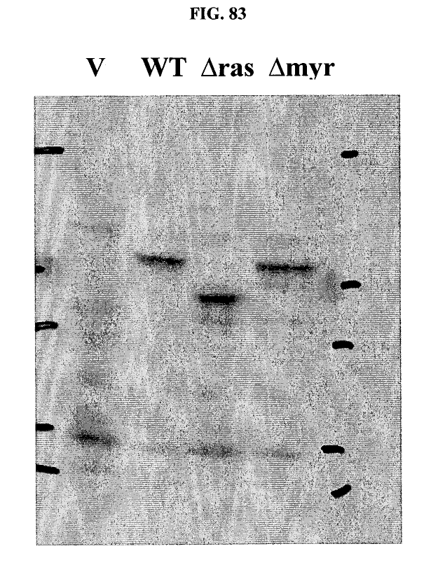

As described above, the AD037 sequence contains several functional motifs including several consensus myristoylation sites near the amino terminus located at amino acid 26-31, amino acid 102-107, and amino acid 186 to 191 of SEQ ID NO:126, in addition to a Ras association motif located at about 172 to about amino acid 262 of SEQ ID NO:126. To determine if these motifs were functional, two deletion mutants (Δmyr, Δras) were generated that deleted either the myristolation site located at amino acid 26-31 or the Ras association motif. The other myristolation sites within the AD037 polypeptide were not investigated relative to their role in AD037 function. As shown in FIG. 83, all three constructs expressed proteins of the appropriate sizes after transfection in Cos cells.

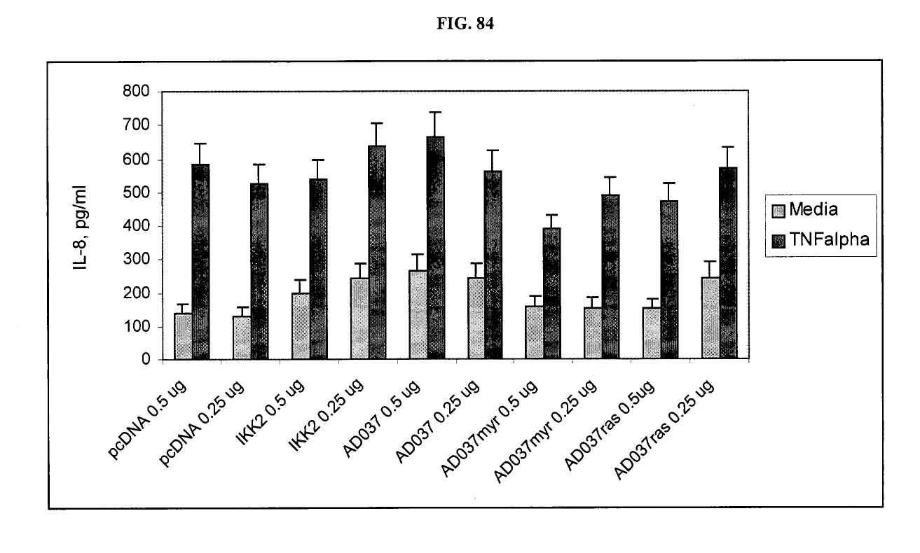

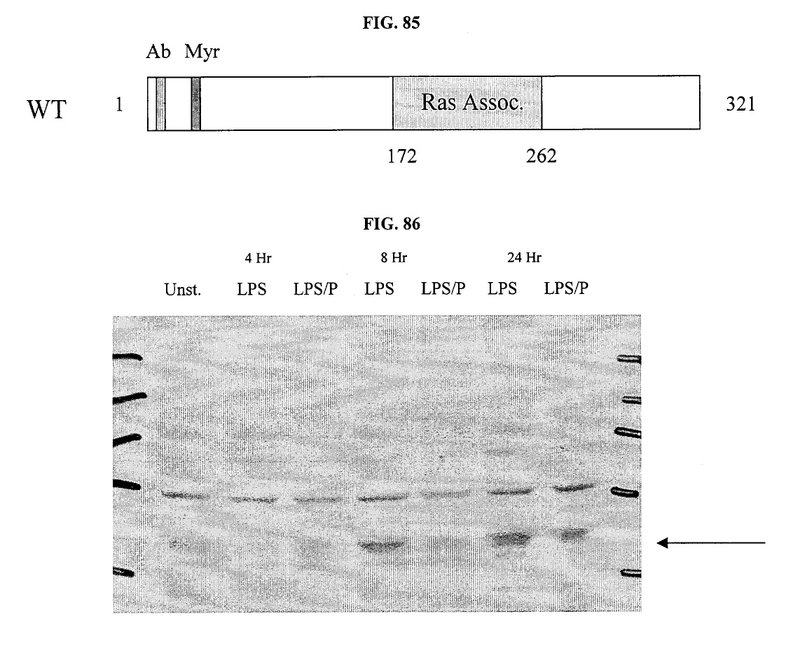

In order to assess whether the Δmyr and Δras AD037 deletion mutants were functional, expression vectors containing the coding regions of each mutant were transfected into H292 epithelial cells and the level of TNFα-induced IL-8 production was measured. Consistent with earlier results, the expression of wild type IKK-2 and wild type AD037 significantly increased basal and induced levels of IL-8 above that detected in cells transfected with vector alone (see FIG. 84). Expression of either the aa26-31 myristoylation site deletion or the Ras Association motif mutant failed to increase IL-8 levels above that detected in the vector controls. The data indicates that both motifs are required for AD037 function.

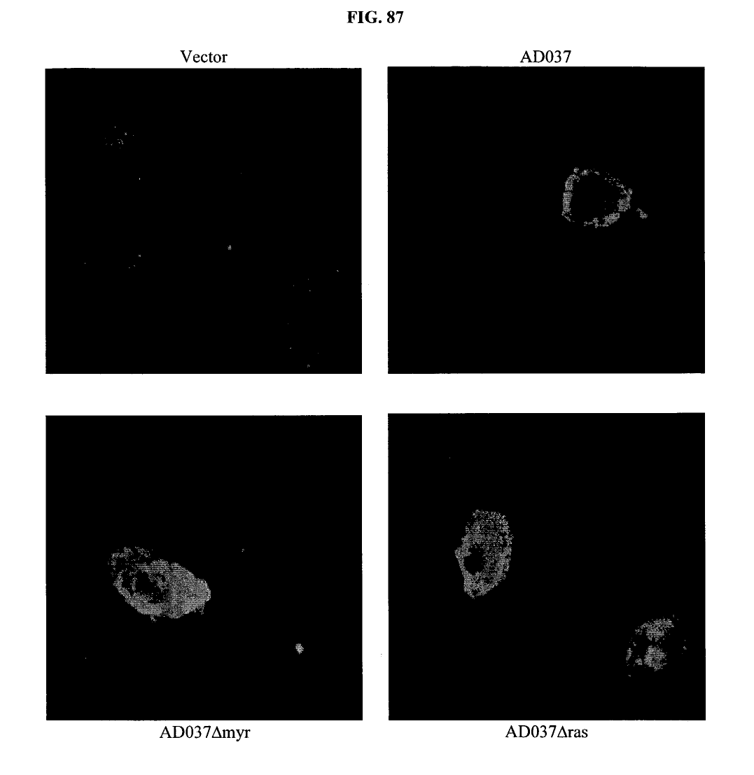

Although the modulation of AD037 expression by NFkB and/or members of the NFkB pathway has been demonstrated at the mRNA level, additional analysis was performed to assess whether the NFkB-dependent regulation was also observable at the level of the AD037 protein. THP-1 monocytes were stimulated with LPS (100 ng/ml) in the presence and absence of BMS-205820 (pep) for 4 to 24 hours. At each time point, cells were harvested and lysed in RIPA buffer as described. Whole cell lysates were electrophoresed through a 4-20% Tris-glycine gel, transferred to nitrocellulose, blocked overnight with 5% non fat dry milk in Tris-buffered saline, and probed with rabbit antisera raised to a peptide containing amino acids 11-24 of SEQ ID NO: 126 (SEQ ID NO:289) AD037. Bands were detected with HRP-tagged anti-rabbit antibodies followed by ECL. As shown in FIG. 86, the level of AD037 protein was inhibited in the presence of the NFkB inhibitory peptide (SEQ ID NO:124). The arrow on FIG. 86 indicates a specific band that was blocked upon preincubation with the rabbit antisera generated with the immunizing AD037 peptide (SEQ ID NO:289). The immunizing peptide was conjugated to KLH through an NH₂-terminal Cys for injection into rabbits. This band corresponds to the AD037 protein.

The association of AD037 to modulating IL-8 expression is consistent with the association of AD037 to the NFkB pathway since IL-8 expression is dependent upon NFkB (Hoffmann et al. (2002) J. Leukoc. Biol. 72:847-855). Thus, in preferred embodiments, AD037 polynucleotides and polypeptides, including modulators and fragments thereof are useful for treating, ameliorating, and/or detecting disorders associated with IL-8, disorders associated with aberrant IL-8 expression, disorders associated with aberrant IL-8 activity, asthma, pulmonary disorders, pulmonary fibrosis, Behcet's disease, bacterial infections, viral infections, gynaecological diseases, psoriasis, inflammatory bowel disease, IgA nephropathy, chronic obstructive pulmonary disease, Kawasaki disease, Crohn's disease, peripheral arterial occlusive disease, Hodgkin's disease, idiopathic intermediate uveitis, hyaline membrane disease, acute rheumatic fever, chronic rheumatic heart disease, ulcerative colitis, autoimmune disorders, and autoimmune thyroid disease.

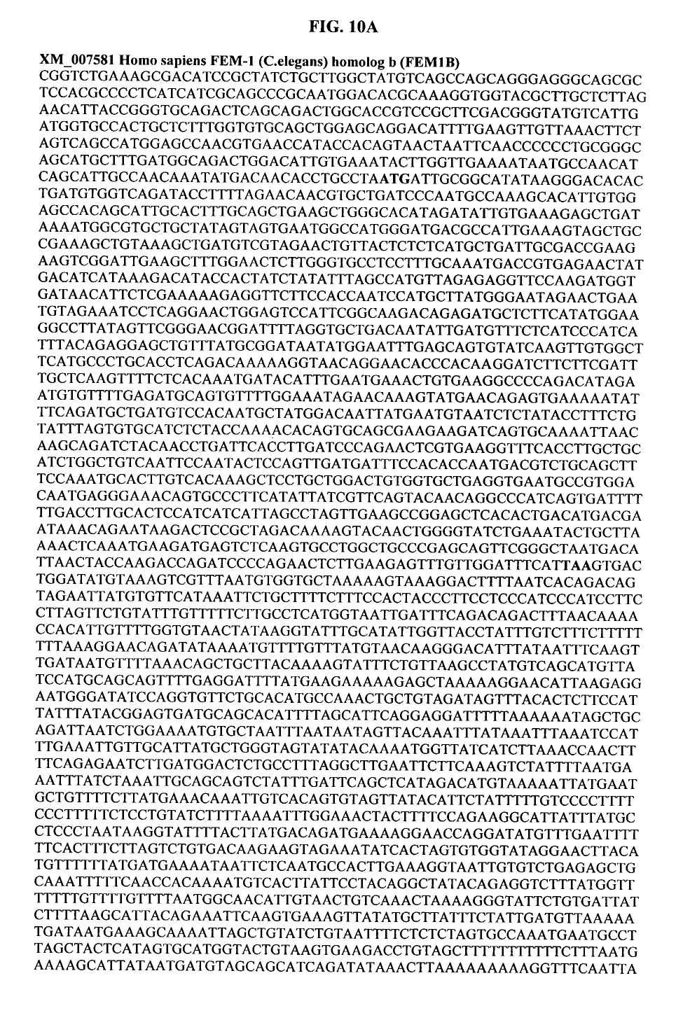

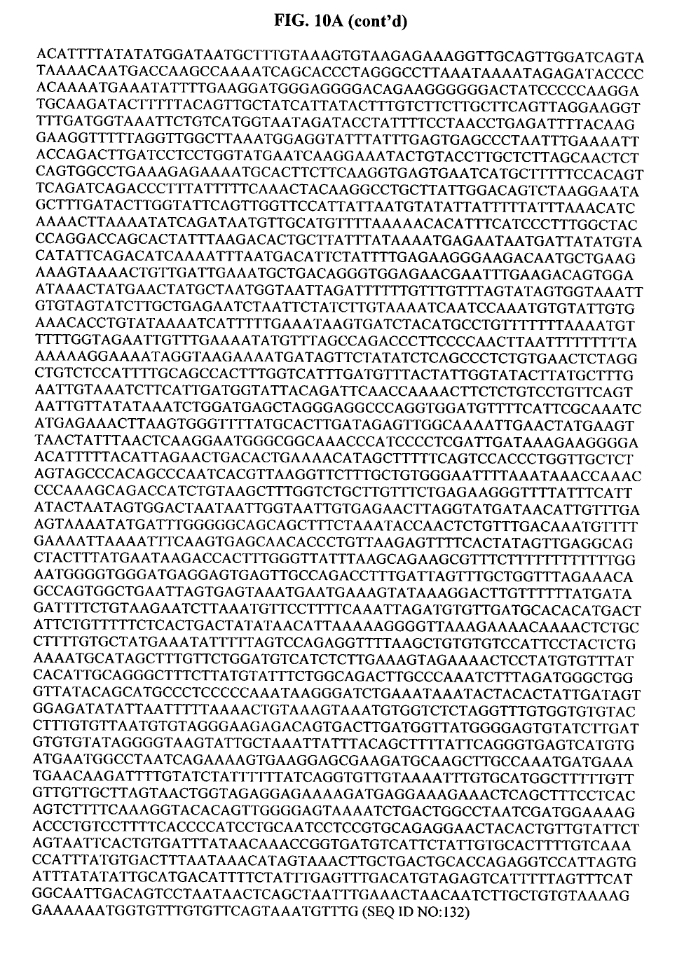

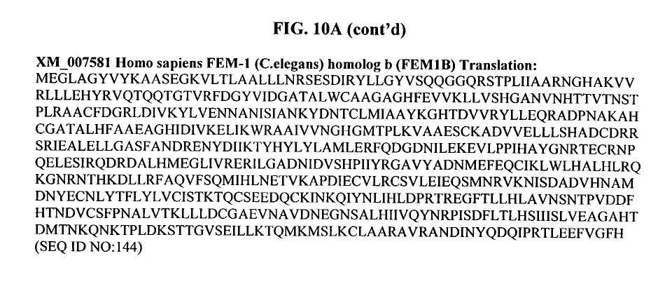

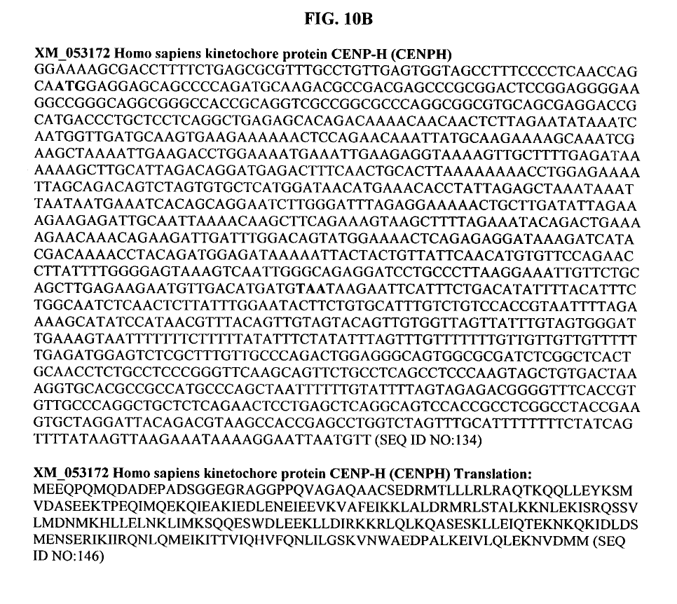

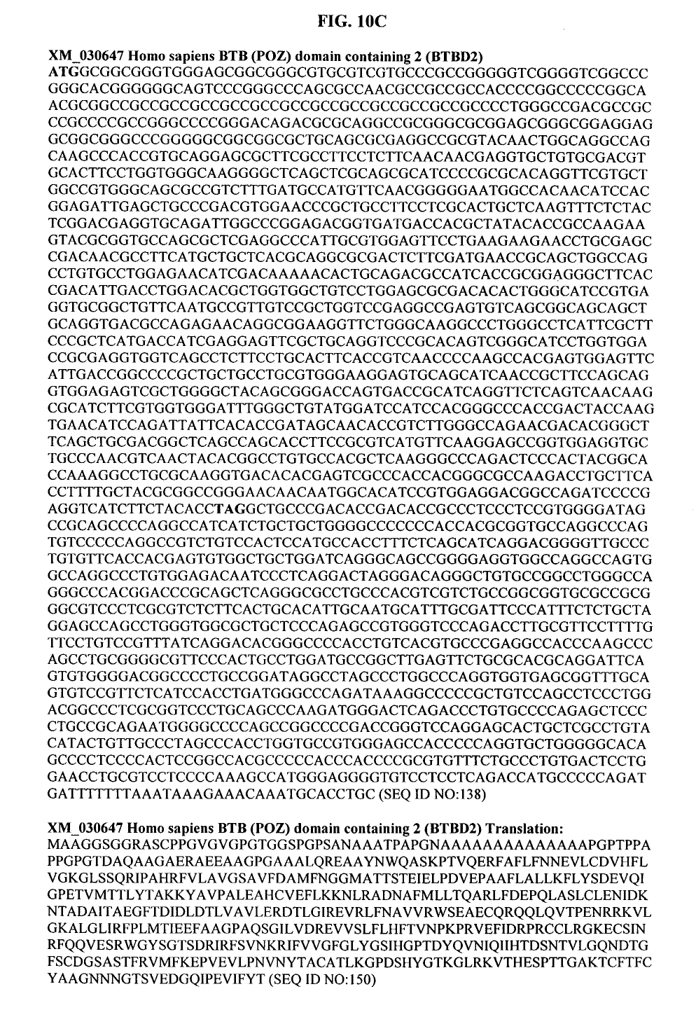

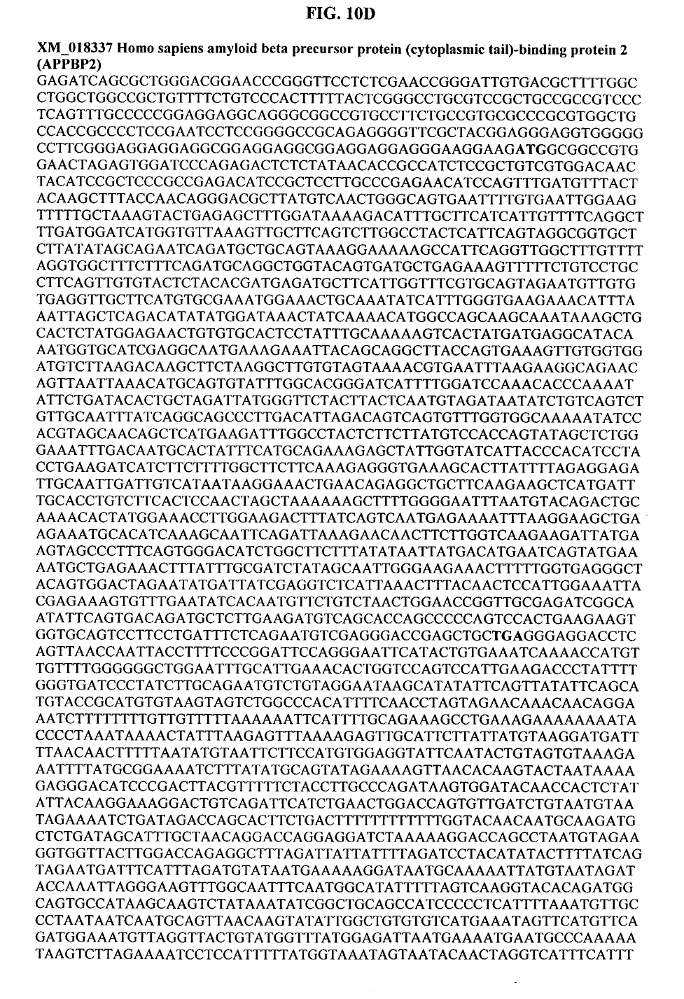

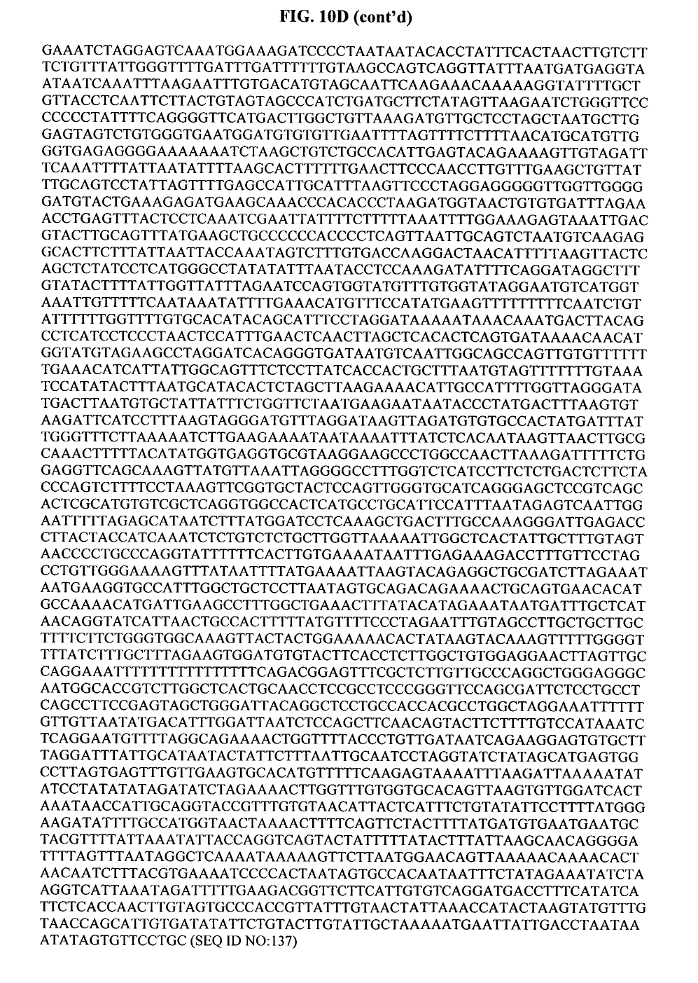

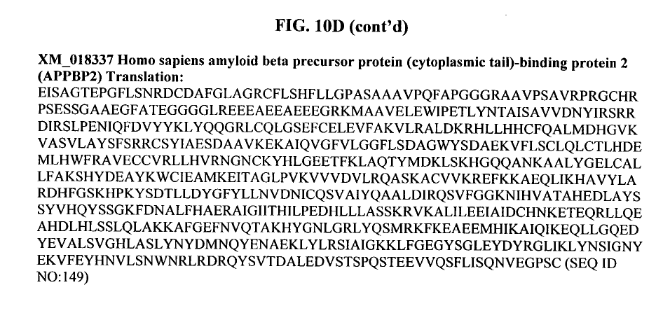

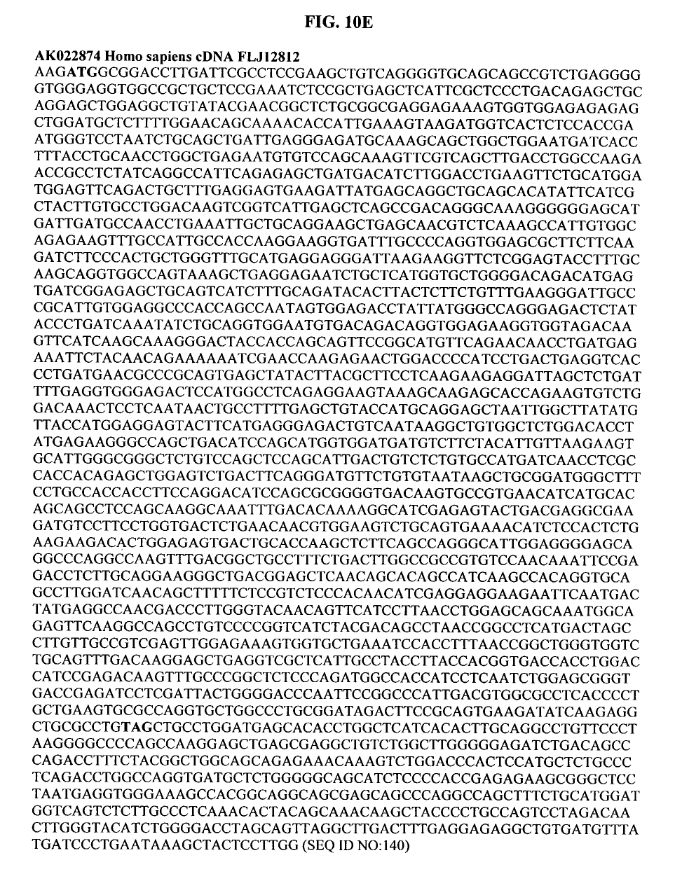

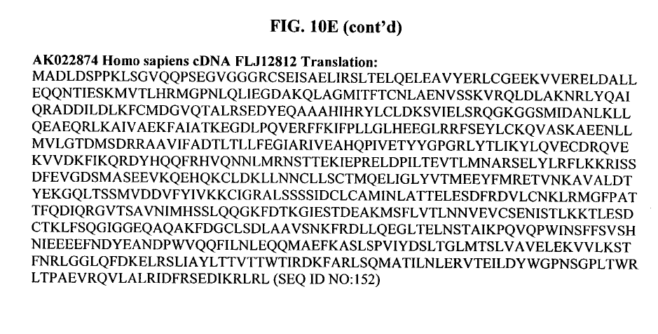

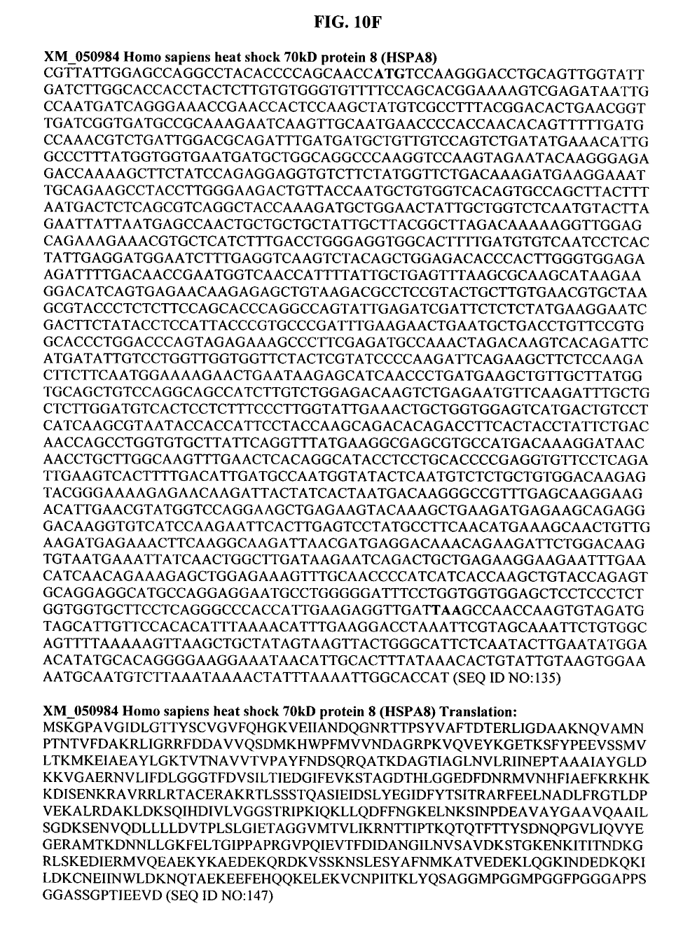

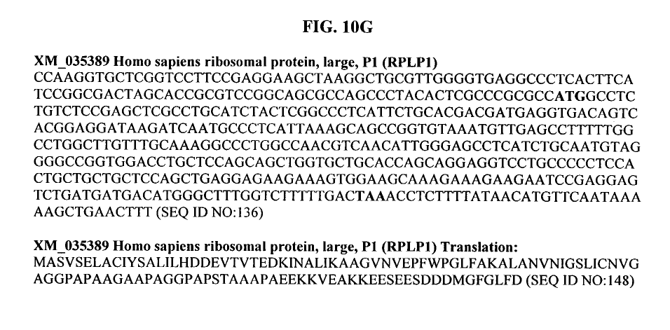

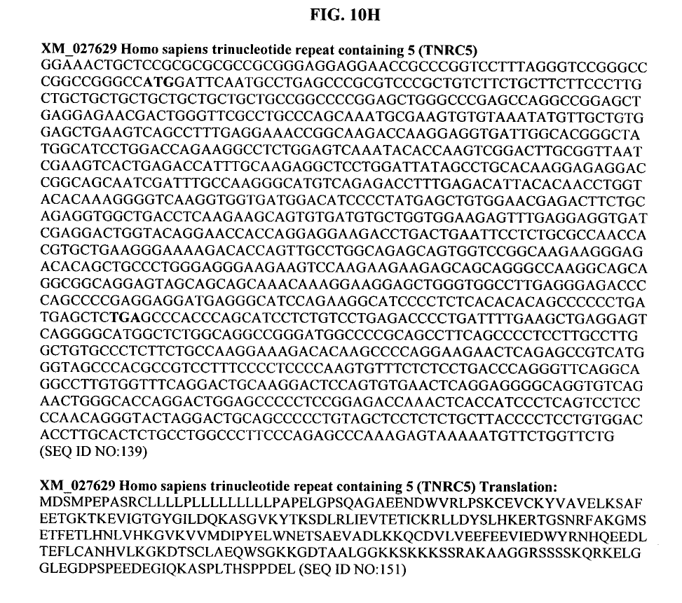

In order to identify pathways/proteins associated with AD037, a yeast two-hybrid screen was performed. Full length AD037 was cloned into a bait vector that was used to screen a library derived from LPS-stimulated THP-1 cells. Eight different interacting clones were isolated and are as follows: FEM-1b, the human homologue to C. elegans FEM-1 (Genbank Accession No: XM_{—}007581; SEQ ID NO:132 and 144); the human kinetochore protein CENP-H (Genbank Accession No: XM_{—}053172; SEQ ID NO:134 and 146); the human heat shock 70 kD protein (HSP70) (Genbank Accession No: XM_{—}050984; SEQ ID NO: 135 and 147); the human large P1 ribosomal protein (Genbank Accession No: XM_{—}035389; SEQ ID NO:136 and 148); the human microtubule binding protein PAT1 (Genbank Accession No: XM_{—}018337; SEQ ID NO: 137 and 149); the human BTB/POZ domain containing protein (Genbank Accession No: XM_{—}030647; SEQ ID NO:138 and 150); the human trinucleotide repeat containing 5 protein (Genbank Accession No: XM_{—}027629; SEQ ID NO:139 and 151); and the human FLJ12812 (Genbank Accession No: AK022874; SEQ ID NO:140 and 152) (see FIGS. 10A-H).

The C. elegans FEM-1 protein is a signal transduction regulator of the sex determination pathway (Ventura-Holman et al. (1998) Genomics 54:221-230). The human FEM-1b homologue contains 8 ankyrin repeats.

CENP-H is a constitutive centrosome component that colocalizes with inner kinetochore plate proteins CENP-A and CENP-C throughout the cell cycle suggesting that it may play a role in kinetochore organization and function (Sugata et al. (2000) Hum. Mol. Genet. 9:2919-2926).

HSP70 is a molecular chaperone involved in protein folding (Bukau et al. (1998) Cell 92:351-366).

The acidic ribosomal P1 protein plays an important role in the elongation step of protein synthesis (Remacha et al. (1995) Biochem. Cell. Biol. 73:959-968).

PAT1 is a microtubule-interacting protein that is involved in the translocation of amyloid precursor protein along microtubules toward the cell surface (Zheng et al. (1998) Proc. Natl. Acad. Sci. USA 95:14745-14750).

The BTB/POZ domain mediates homomeric dimerization, and in some cases heterodimeric dimerization. This domain is found in several zinc finger containing proteins that function as transcriptional repressors (Zollman et al. (1994) Proc. Natl. Acad. Sci. USA 91:10717-10721).

Trinucleotide repeat containing 5 protein is a member of a family of trinucleotide repeat expansion mutants, twelve of which have been associated with human diseases (Margolis et al. (1997) Hum. Genet. 100:114-122).

The hypothetical protein FLJ12812 contains a domain shared by the Bcl-2 interactor beclin 1, and the Schizosaccharomyces pombe protein required for chromosome condensation and segregation.

The ability of AD037 to interact with proteins that regulate kinetochore function, protein elongation, and protein translocation suggests that AD037 may regulate protein synthesis and transport in response to cell cycle signals. In addition, it is clear that the pathway associated with AD037 is important in inflammatory diseases. Such a use is consistent with the elevation of AD037 expression levels in synovial samples derived from rheumatoid arthritis patients as compared to osteoarthritis synovium, and in comparison to synovium derived from joint trauma controls (see FIG. 6). Increased expression of an NF-kB target gene in rheumatoid arthritis synovium is consistent with the constitutive activation of NF-kB that has been previously described in rheumatoid arthritis. This result further suggests that the target genes identified using the yeast two-hybrid system may play important roles in diseases associated with aberrant NF-kB activation including rheumatoid arthritis, inflammatory bowel disease, asthma, atherosclerosis, cachexia, stroke, and cancer, among others.

The confirmation that the expression of the AD037 polynucleotide and encoded peptide are inhibited by NFkB suggests that antagonists directed against the AD037 polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the AD037 polynucleotide and/or encoded peptide are useful for decreasing NF-kB activity, decreasing apoptotic events, and/or increasing IkBa expression or activity levels.

The AD037 NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The AD037 NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

In preferred embodiments, the following N-terminal AD037 deletion polypeptides are encompassed by the present invention: M1-K321, K2-K321, E3-K321, D4-K321, C5-K321, L6-K321, P7-K321, S8-K321, S9-K321, H10-K321, V11-K321, P12-K321, I13-K321, S14-K321, D15-K321, S16-K321, K17-K321, S18-K321, I19-K321, Q20-K321, K21-K321, S22-K321, E23-K321, L24-K321, L25-K321, G26-K321, L27-K321, L28-K321, K29-K321, T30-K321, Y31-K321, N32-K321, C33-K321, Y34-K321, H35-K321, E36-K321, G37-K321, K38-K321, S39-K321, F40-K321, Q41-K321, L42-K321, R43-K321, H44-K321, R45-K321, E46-K321, E47-K321, E48-K321, G49-K321, T50-K321, L51-K321, I52-K321, I53-K321, E54-K321, G55-K321, L56-K321, L57-K321, N58-K321, I59-K321, A60-K321, W61-K321, G62-K321, L63-K321, R64-K321, R65-K321, P66-K321, I67-K321, R68-K321, L69-K321, Q70-K321, M71-K321, Q72-K321, D73-K321, D74-K321, R75-K321, E76-K321, Q77-K321, V78-K321, H79-K321, L80-K321, P81-K321, S82-K321, T83-K321, S84-K321, W85-K321, M86-K321, P87-K321, R88-K321, R89-K321, P90-K321, S91-K321, C92-K321, P93-K321, L94-K321, K95-K321, E96-K321, P97-K321, S98-K321, P99-K321, Q100-K321, N101-K321, G102-K321, N103-K321, I104-K321, T105-K321, A106-K321, K107-K321, G108-K321, P109-K321, S110-K321, I111-K321, Q112-K321, P113-K321, V114-K321, H115-K321, K116-K321, A117-K321, E118-K321, S119-K321, S120-K321, T121-K321, D122-K321, S123-K321, S124-K321, G125-K321, P126-K321, L127-K321, E128-K321, E129-K321, A130-K321, E131-K321, E132-K321, A133-K321, P134-K321, Q135-K321, L136-K321, M137-K321, R138-K321, T139-K321, K140-K321, S141-K321, D142-K321, A143-K321, S144-K321, C145-K321, M146-K321, S147-K321, Q148-K321, R149-K321, R150-K321, P151-K321, K152-K321, C153-K321, R154-K321, A155-K321, P156-K321, G157-K321, E158-K321, A159-K321, Q160-K321, R161-K321, I162-K321, R163-K321, R164-K321, H165-K321, R166-K321, F167-K321, S168-K321, I169-K321, N170-K321, G171-K321, H172-K321, F173-K321, Y174-K321, N175-K321, H176-K321, K177-K321, T178-K321, S179-K321, V180-K321, F181-K321, T182-K321, P183-K321, A184-K321, Y185-K321, G186-K321, S187-K321, V188-K321, T189-K321, N190-K321, V191-K321, R192-K321, V193-K321, N194-K321, S195-K321, T196-K321, M197-K321, T198-K321, T199-K321, L200-K321, Q201-K321, V202-K321, L203-K321, T204-K321, L205-K321, L206-K321, L207-K321, N208-K321, K209-K321, F210-K321, R211-K321, V212-K321, E213-K321, D214-K321, G215-K321, P216-K321, S217-K321, E218-K321, F219-K321, A220-K321, L221-K321, Y222-K321, I223-K321, V224-K321, H225-K321, E226-K321, S227-K321, G228-K321, E229-K321, R230-K321, T231-K321, K232-K321, L233-K321, K234-K321, D235-K321, C236-K321, E237-K321, Y238-K321, P239-K321, L240-K321, 1241-K321, S242-K321, R243-K321, 1244-K321, L245-K321, H246-K321, G247-K321, P248-K321, C249-K321, E250-K321, K251-K321, I252-K321, A253-K321, R254-K321, I255-K321, F256-K321, L257-K321, M258-K321, E259-K321, A260-K321, D261-K321, L262-K321, G263-K321, V264-K321, E265-K321, V266-K321, P267-K321, H268-K321, E269-K321, V270-K321, A271-K321, Q272-K321, Y273-K321, I274-K321, K275-K321, F276-K321, E277-K321, M278-K321, P279-K321, V280-K321, L281-K321, D282-K321, S283-K321, F284-K321, V285-K321, E286-K321, K287-K321, L288-K321, K289-K321, E290-K321, E291-K321, E292-K321, E293-K321, R294-K321, E295-K321, I296-K321, I297-K321, K298-K321, L299-K321, T300-K321, M301-K321, K302-K321, F303-K321, Q304-K321, A305-K321, L306-K321, R307-K321, L308-K321, T309-K321, M310-K321, L311-K321, Q312-K321, R313-K321, L314-K321, and/or E315-K321 of SEQ ID NO: 126. Polynucleotide sequences encoding these polypeptides are also provided. The present invention also encompasses the use of these N-terminal AD037 deletion polypeptides as immunogenic and/or antigenic epitopes as described elsewhere herein.

In preferred embodiments, the following C-terminal AD037 deletion polypeptides are encompassed by the present invention: M1-K321, M1-A320, M1-E319, M1-V318, M1-L317, M1-Q316, M1-E315, M1-L314, M1-R313, M1-Q312, M1-L311, M1-M310, M1-T309, M1-L308, M1-R307, M1-L306, M1-A305, M1-Q304, M1-F303, M1-K302, M1-M301, M1-T300, M1-L299, M1-K298, M1-I297, M1-I296, M1-E295, M1-R294, M1-E293, M1-E292, M1-E291, M1-E290, M1-K289, M1-L288, M1-K287, M1-E286, M1-V285, M1-F284, M1-S283, M1-D282, M1-L281, M1-V280, M1-P279, M1-M278, M1-E277, M1-F276, M1-K275, M1-I274, M1-Y273, M1-Q272, M1-A271, M1-V270, M1-E269, M1-H268, M1-P267, M1-V266, M1-E265, M1-V264, M1-G263, M1-L262, M1-D261, M1-A260, M1-E259, M1-M258, M1-L257, M1-F256, M1-I255, M1-R254, M1-A253, M1-I252, M1-K251, M1-E250, M1-C249, M1-P248, M1-G247, M1-H246, M1-L245, M1-I244, M1-R243, M1-S242, M1-I241, M1-L240, M1-P239, M1-Y238, M1-E237, M1-C236, M1-D235, M1-K234, M1-L233, M1-K232, M1-T231, M1-R230, M1-E229, M1-G228, M1-S227, M1-E226, M1-H225, M1-V224, M1-I223, M1-Y222, M1-L221, M1-A220, M1-F219, M1-E218, M1-S217, M1-P216, M1-G215, M1-D214, M1-E213, M1-V212, M1-R211, M1-F210, M1-K209, M1-N208, M1-L207, M1-L206, M1-L205, M1-T204, M1-L203, M1-V202, M1-Q201, M1-L200, M1-T199, M1-T198, M1-M197, M1-T196, M1-S195, M1-N194, M1-V193, M1-R192, M1-V191, M1-N190, M1-T189, M1-V188, M1-S187, M1-G186, M1-Y185, M1-A184, M1-P183, M1-T182, M1-F181, M1-V180, M1-S179, M1-T178, M1-K177, M1-H176, M1-N175, M1-Y174, M1-F173, M1-H172, M1-G171, M1-N170, M1-I169, M1-S168, M1-F167, M1-R166, M1-H165, M1-R164, M1-R163, M1-I162, M1-R161, M1-Q160, M1-A159, M1-E158, M1-G157, M1-P156, M1-A155, M1-R154, M1-C153, M1-K152, M1-P151, M1-R150, M1-R149, M1-Q148, M1-S147, M1-M146, M1-C145, M1-S144, M1-A143, M1-D142, M1-S141, M1-K140, M1-T139, M1-R138, M1-M137, M1-L136, M1-Q135, M1-P134, M1-A133, M1-E132, M1-E131, M1-A130, M1-E129, M1-E128, M1-L127, M1-P126, M1-G125, M1-S124, M1-S123, M1-D122, M1-T121, M1-S120, M1-S119, M1-E118, M1-A117, M1-K116, M1-H115, M1-V114, M1-P113, M1-Q112, M1-I111, M1-S110, M1-P109, M1-G108, M1-K107, M1-A106, M1-T105, M1-I104, M1-N103, M1-G102, M1-N101, M1-Q100, M1-P99, M1-S98, M1-P97, M1-E96, M1-K95, M1-L94, M1-P93, M1-C92, M1-S91, M1-M1-R89, M1-R88, M1-P87, Ml-M86, M1-W85, M1-S84, M1-T83, M1-S82, M1-P81, M1-L80, M1-H79, M1-V78, M1-Q77, M1-E76, M1-R75, M1-D74, M1-D73, M1-Q72, M1-M71, M1-Q70, M1-L69, M1-R68, M1-167, M1-P66, M1-R64, M1-L63, M1-G62, M1-W61, M1-A60, M1-159, M1-N58, M1-L57, M1-L56, M1-G55, M1-E54, M1-153, M1-I52, M1-L51, M1-T50, M1-G49, M1-E48, M1-E47, M1-E46, M1-R45, M1-H44, M1-R43, M1-L42, M1-Q41, M1-F40, M1-S39, M1-K38, M1-G37, M1-E36, M1-H35, M1-Y34, M1-C33, M1-N32, M1-Y31, M1-T30, M1-K29, M1-L28, M1-L27, M1-G26, M1-L25, M1-L24, M1-E23, M1-S22, M1-K21, M1-Q20, M1-I19, M1-S18, M1-K17, M1-S16, M1-D15, M1-S14, M1-I13, M1-P12, M1-V11, M1-H10, M1-S9, M1-S8, and/or M1-P7 of SEQ ID NO:126. Polynucleotide sequences encoding these polypeptides are also provided. The present invention also encompasses the use of these C-terminal AD037 deletion polypeptides as immunogenic and/or antigenic epitopes as described elsewhere herein.

Alternatively, preferred polypeptides of the present invention may comprise polypeptide sequences corresponding to, for example, internal regions of the AD037 polypeptide (e.g., any combination of both N- and C-terminal AD037 polypeptide deletions) of SEQ ID NO:126. For example, internal regions could be defined by the equation: amino acid NX to amino acid CX, wherein NX refers to any N-terminal deletion polypeptide amino acid of AD037 (SEQ ID NO:126), and where CX refers to any C-terminal deletion polypeptide amino acid of AD037 (SEQ ID NO:126). Polynucleotides encoding these polypeptides are also provided. The present invention also encompasses the use of these polypeptides as an immunogenic and/or antigenic epitope as described elsewhere herein.

The present invention also encompasses immunogenic and/or antigenic epitopes of the AD037 polypeptide.

The AD037 polypeptides of the present invention were determined to comprise several phosphorylation sites based upon the Motif algorithm (Genetics Computer Group, Inc.). The phosphorylation of such sites may regulate some biological activity of the AD037 polypeptide. For example, phosphorylation at specific sites may be involved in regulating the proteins ability to associate or bind to other molecules (e.g., proteins, ligands, substrates, DNA, etc.). In the present case, phosphorylation may modulate the ability of the AD037 polypeptide to associate with other polypeptides, particularly cognate ligand for AD037, or its ability to modulate certain cellular signal pathways.

The AD037 polypeptide was predicted to comprise three PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: QNGNITAKGPSIQ (SEQ ID NO:290), DASCMSQRRPKCR (SEQ ID NO:291), and/or EIIKLTMKFQALR (SEQ ID NO:292). Polynucleotides encoding these polypeptides are also provided. The present invention also encompasses the use of the AD037 PKC phosphorylation site polypeptides as immunogenic and/or antigenic epitopes as described elsewhere herein.

The AD037 polypeptide was predicted to comprise three casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to aminoacyl-transfer RNA synthetases class-II domains may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: VHKAESSTDSSGPL (SEQ ID NO:293), PQLMRTKSDASCMS (SEQ ID NO:294), and/or MPVLDSFVEKLKEE (SEQ ID NO:295). Polynucleotides encoding these polypeptides are also provided. The present invention also encompasses the use of this casein kinase II phosphorylation site polypeptide as an immunogenic and/or antigenic epitope as described elsewhere herein.

The AD037 polypeptide was predicted to comprise two cAMP- and cGMP-dependent protein kinase phosphorylation site using the Motif algorithm (Genetics Computer Group, Inc.). There has been a number of studies relative to the specificity of cAMP- and cGMP-dependent protein kinases. Both types of kinases appear to share a preference for the phosphorylation of serine or threonine residues found close to at least two consecutive N-terminal basic residues.

A consensus pattern for cAMP- and cGMP-dependent protein kinase phosphorylation sites is as follows: [RK](2)-x-[ST], wherein “x” represents any amino acid, and S or T is the phosphorylation site.

Additional information specific to cAMP- and cGMP-dependent protein kinase phosphorylation sites may be found in reference to the following publication: Fremisco J. R., Glass D. B., Krebs E. G, J. Biol. Chem. 255:4240-4245(1980); Glass D. B., Smith S. B., J. Biol. Chem. 258:14797-14803(1983); and Glass D. B., El-Maghrabi M. R., Pilkis S. J., J. Biol. Chem. 261:2987-2993(1986); which is hereby incorporated herein in its entirety.

In preferred embodiments, the following cAMP- and cGMP-dependent protein kinase phosphorylation site polypeptide is encompassed by the present invention: TSWMPRRPSCPLKE (SEQ ID NO:296). Polynucleotides encoding this polypeptide are also provided. The present invention also encompasses the use of this cAMP- and cGMP-dependent protein kinase phosphorylation site polypeptide as an immunogenic and/or antigenic epitope as described elsewhere herein.

Specifically, the AD037 polypeptide was predicted to comprise one tyrosine phosphorylation site using the Motif algorithm (Genetics Computer Group, Inc.). Such sites are phosphorylated at the tyrosine amino acid residue. The consensus pattern for tyrosine phosphorylation sites are as follows: [RK]-x(2)-[DE]-x(3)-Y, or or [RK]-x(3)-[DE]-x(2)-Y, where Y represents the phosphorylation site and ‘x’ represents an intervening amino acid residue. Additional information specific to tyrosine phosphorylation sites can be found in Patschinsky T., Hunter T., Esch F. S., Cooper J. A., Sefton B. M., Proc. Nati. Acad. Sci. U.S.A. 79:973-977(1982); Hunter T., J. Biol. Chem. 257:4843-4848(1982), and Cooper J. A., Esch F. S., Taylor S. S., Hunter T., J. Biol. Chem. 259:7835-7841(1984), which are hereby incorporated herein by reference.

In preferred embodiments, the following tyrosine phosphorylation site polypeptide is encompassed by, the present invention: SGERTKLKDCEYPLISR (SEQ ID NO:302). Polynucleotides encoding these polypeptides are also provided. The present invention also encompasses the use of this AD037 tyrosine phosphorylation site polypeptide as an immunogenic and/or antigenic epitopes as described elsewhere herein.

The AD037 polypeptide has been shown to comprise two glycosylation site according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following asparagine glycosylation site polypeptides are encompassed by the present invention: SPQNGNITAKGPSI (SEQ ID NO:297), and/or TNVRVNSTMTTLQV (SEQ ID NO:298). Polynucleotides encoding these polypeptides are also provided. The present invention also encompasses the use of these AD037 asparagine glycosylation site polypeptide as immunogenic and/or antigenic epitopes as described elsewhere herein.

The AD037 polypeptide was predicted to comprise three N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to cAMP- and cGMP-dependent protein kinase phosphorylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: KSELLGLLKTYNCYHE (SEQ ID NO:299), PSPQNGNITAKGPSIQ (SEQ ID NO:300), and/or FTPAYGSVTNVRVNST (SEQ ID NO:301). Polynucleotides encoding these polypeptides are also provided. The present invention also encompasses the use of these N-myristoylation site polypeptides as immunogenic and/or antigenic epitopes as described elsewhere herein.

### Features of the Polypeptide Encoded by Gene No:110

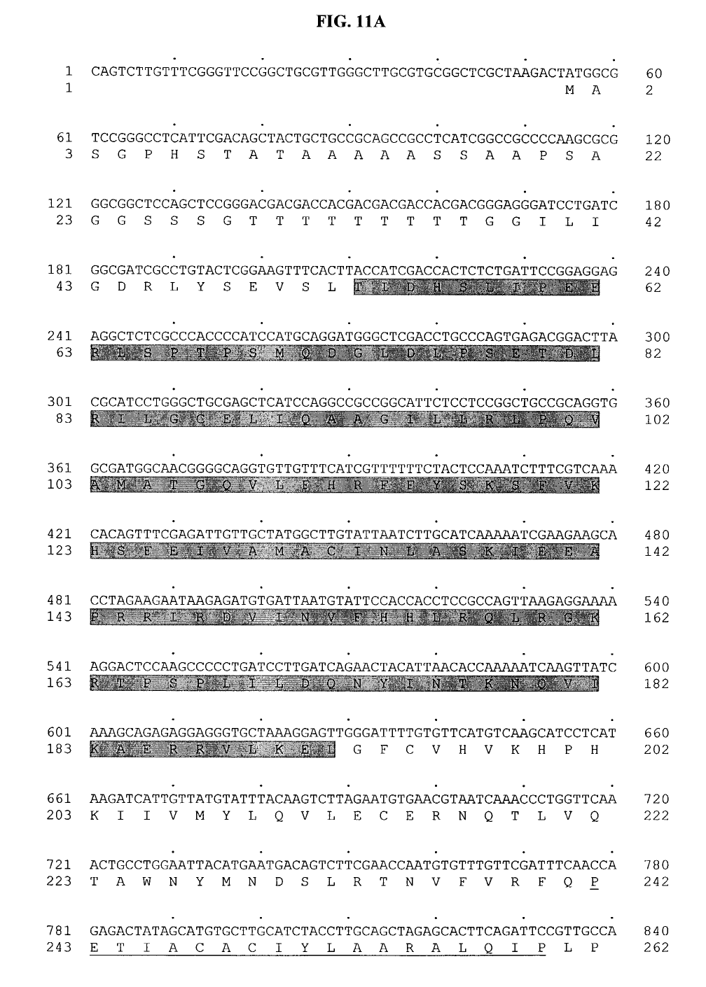

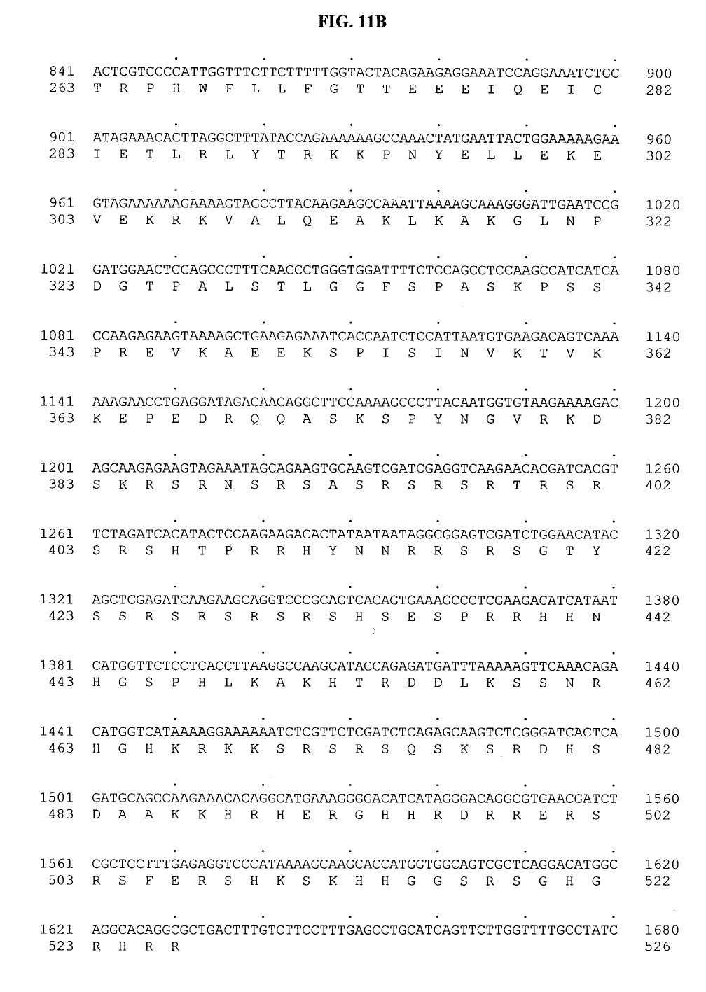

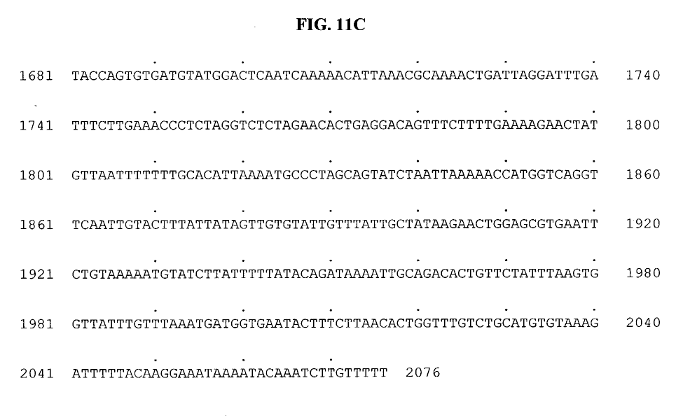

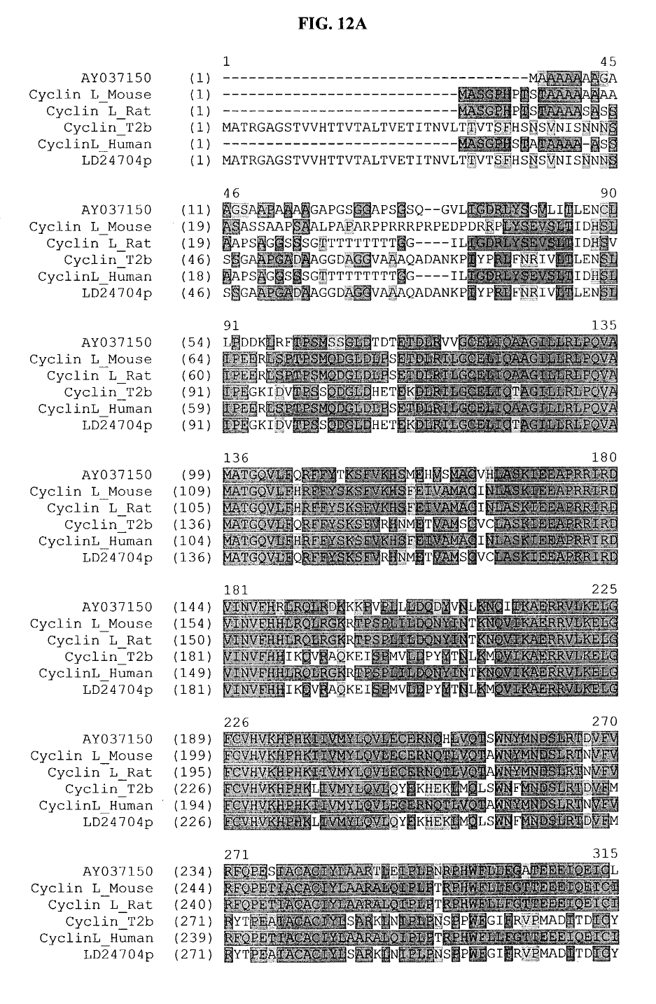

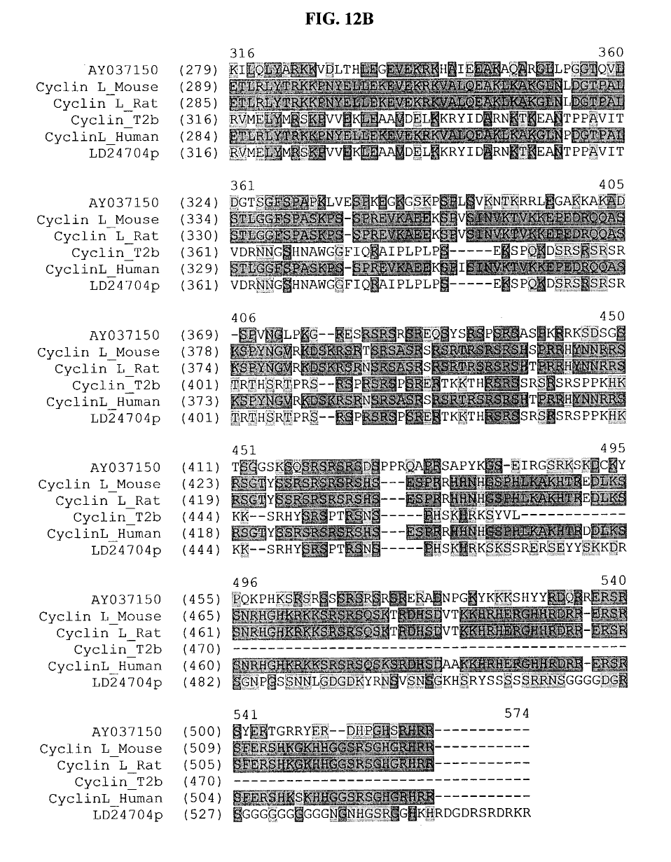

The polypeptide of this gene provided as SEQ ID NO:127 (FIGS. 11A-C), encoded by the polynucleotide sequence according to SEQ ID NO:128 (FIGS. 11A-C), has significant homology at the nucleotide and amino acid level to the rat cyclin L ortholog (Cyclin_L_Rat; Genbank Accession No. gil16758476; SEQ ID NO:153), the mouse cyclin L ortholog (Cyclin_L_Mou; Genbank Accession No. gil5453421; SEQ ID NO:154), the human protein AY037150 (AY037150; Genbank Accession No. gil14585859; SEQ ID NO:155), the Drosophila protein LD24704p (LD24704p; Genbank Accession No. gil16198007; SEQ ID NO:156), and the human cyclin T2b protein (Cyclin_T2b; Genbank Accession No. gil6691833; SEQ ID NO:157). An alignment of the Cyclin L polypeptide with these proteins is provided in FIGS. 12A-B.

The determined nucleotide sequence of the Cyclin L cDNA in FIGS. 11A-C (SEQ ID NO: 127) contains an open reading frame encoding a protein of about 526 amino acid residues, with a deduced molecular weight of about 59.6 kDa. The amino acid sequence of the predicted Cyclin L polypeptide is shown in FIGS. 11A-C (SEQ ID NO: 128). The Cyclin L protein shown in FIGS. 11A-C was determined to share significant identity and similarity to several proteins. Specifically, the AD037 protein shown in FIGS. 2A-C was determined to be about 98% identical and 98% similar to the rat cyclin L ortholog (Cyclin_L_Rat; Genbank Accession No. gil16758476; SEQ ID NO:153), to be about 93% identical and 93% similar to the mouse cyclin L ortholog (Cyclin_L_Mou; Genbank Accession No. gil5453421; SEQ ID NO:154), to be about 62% identical and 69% similar to the human protein AY037150 (AY037150; Genbank Accession No. gil14585859; SEQ ID NO:155), to be about 52% identical and 61% similar to the Drosophila protein LD24704p (LD24704p; Genbank Accession No. gil16198007; SEQ ID NO:156), and to be about 48% identical and 56% similar to the human cyclin T2b protein (Cyclin_T2b; Genbank Accession No. gil6691833; SEQ ID NO:157).

The human cyclin T2b pairs with the cyclin-dependent kinase CDK9 to form the positive transcription elongation factor b (FIG. 3, Peng et al. (1998) Genes Dev. 12:755-762).

Analysis of the Cyclin L polypeptide determined that it contained an N-terminal cyclin motif located at about amino acid 53 to about amino acid 197 of SEQ ID NO:128. The presence of this domain is consistent with cyclin L representing a cyclin protein and its potential involvement in cell cycle processes. Additionally, it was also determined that the Cyclin L polypeptide contained a factor TFIIB repeat motif located at about amino acid 242 to about amino acid 260 of SEQ ID NO:128. The presence of this domain further suggests the involvment of cyclin L in cell cycle processes and specifically with transcription.

In preferred embodiments, the following N-terminal cyclin motif polypeptide is encompassed by the present invention: TIDHSLIPEERLSPTPSMQDGLDLPSETDLRILGCELIQAAGILLRLPQVAMATG QVLFHRFFYSKSFVKHSFEIVAMACINLASKIEEAPRRIRDVINVFHHLRQLRG KRTPSPLILDQNYINTKNQVIKAERRVLKELGFCVH (SEQ ID NO: 142). Polynucleotides encoding this polypeptide are also provided. The present invention also encompasses the use of this Cyclin L N-terminal cyclin motif polypeptide as an immunogenic and/or antigenic epitope as described elsewhere herein.

In preferred embodiments, the following factor TFIIB repeat motif polypeptide is encompassed by the present invention: PETIACACIYLAARALQIP (SEQ ID NO:143). Polynucleotides encoding this polypeptide are also provided. The present invention also encompasses the use of this Cyclin L factor TFIIB repeat motif polypeptide as an immunogenic and/or antigenic epitope as described elsewhere herein.

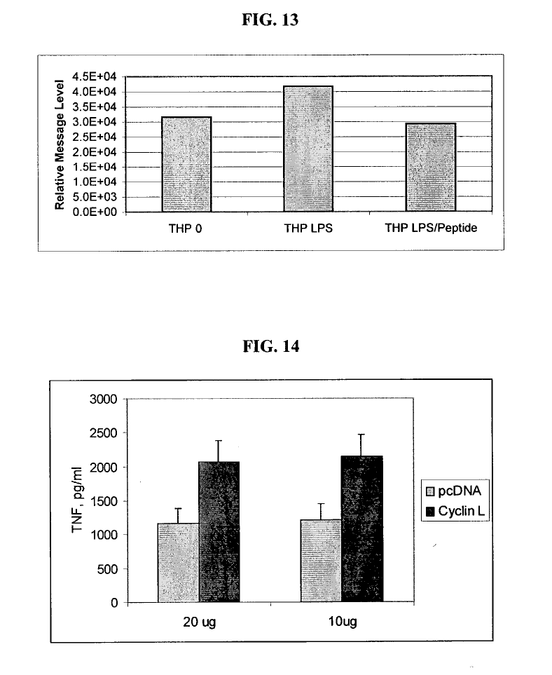

In confirmation that the Cyclin L polypeptide is involved in the NF-kB pathway, real-time PCR analyses was used to show that Cyclin L expression is NF-kb-dependent, as shown in FIG. 13. Cyclin L was expressed in unstimulated THP-1 monocytes as a control. In response to stimulation with LPS, steady-state levels of Cyclin L mRNA increased. This increase in expression was inhibited by inclusion of the selective NF-kB inhibitor, BMS-205820. When Cyclin L was overexpressed in THP-1 monocytes, Cyclin L significantly inhibited TNFα secretion, suggesting that it plays a role in this NF-kB-dependent response, as shown in FIG. 14.

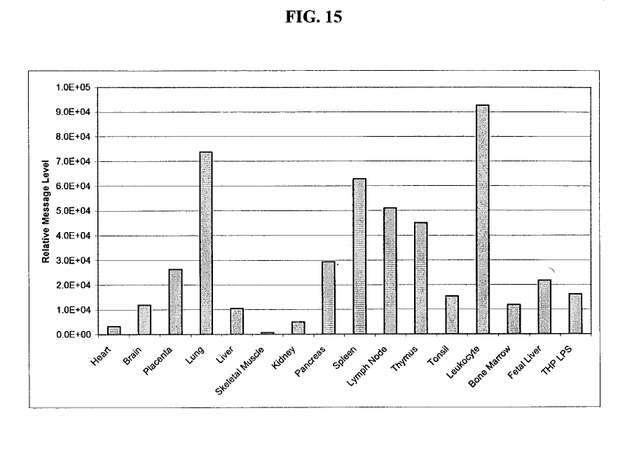

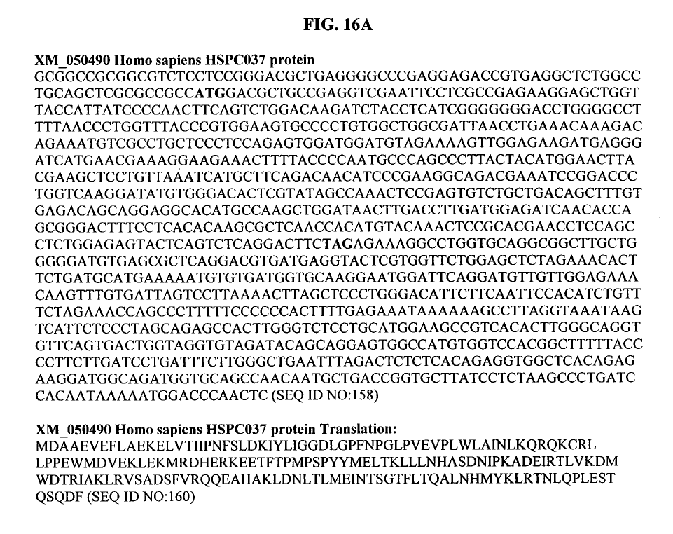

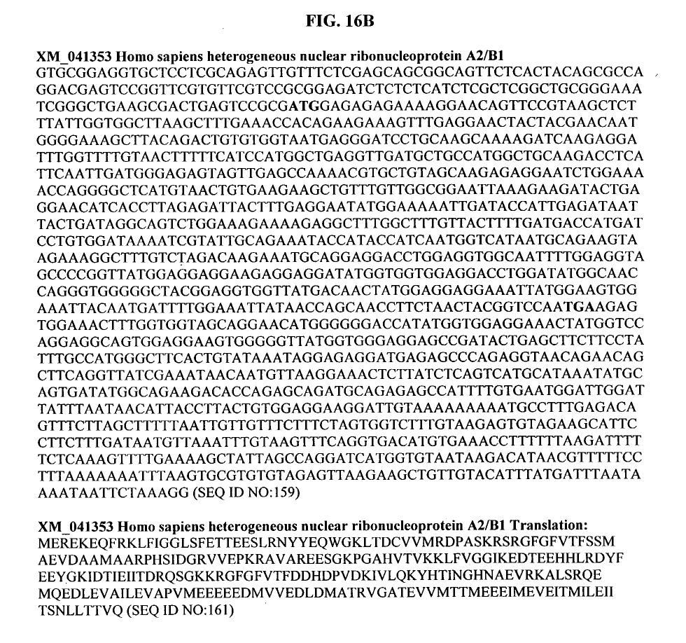

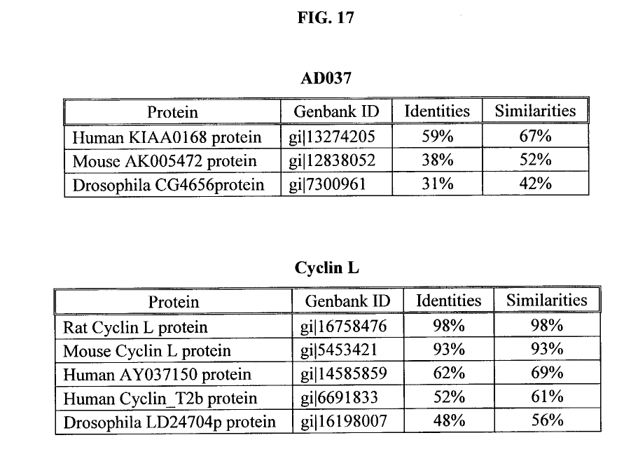

In an effort to further identify additional associations with the NF-kB pathway in other tissues, RT-PCR was performed on a variety of tissues. The results of these experiments indicate that Cyclin L mRNA is expressed at predominately high levels in hematopoietic tissues including leukocytes, spleen, lymph node and thymus. Significant levels were detected in tonsil, bone marrow, and fetal liver, and to a lesser extent in lung, followed by lower levels in pancreas, placenta, liver, brain, kidney, heart, and skeletal muscle (see FIG. 15). The increased expression levels in immune tissues is consistent with the Cyclin L representing a NFkB modulated polynucleotide and polypeptide.

The predominate expression in leukocytes, spleen, lymph node and thymus tissue, in combination with its association with the NFkB pathway suggests the Cyclin L polynucleotides and polypeptides, preferably antagonists, may be useful in treating, diagnosing, prognosing, and/or preventing immune diseases and/or disorders. Representative uses are described in the “Immune Activity”, “Chemotaxis”, and “Infectious Disease” sections below, and elsewhere herein. Briefly, the strong expression in immune tissue indicates a role in regulating the proliferation; survival; differentiation; activation of hematopoietic cell lineages, including blood stem cells, immune deficiencies, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma, and modulating cytokine production, antigen presentation, or other processes, such as for boosting immune responses.

In order to identify pathways/proteins associated with Cyclin L, a yeast two-hybrid screen was performed. Full length Cyclin L was cloned into a bait vector that was used to screen a library derived from LPS-stimulated THP-1 cells. Two different interacting clones were isolated and are as follows: the human HSPC037 protein (Genbank Accession No: XM_{—}050490; SEQ ID NO:158 and 160); and the human heterogeneous nuclear ribonucleoprotein A2/B1 (Genbank Accession No: XM_{—}041353; SEQ ID NO:159 and 161) (FIGS. 16A-B).

The heterogeneous ribonucleoprotein A2/B1 shuttles between the nucleus and cytosol, and plays a role in mRNA packaging, processing and export (Mili et al. (2001) Mol. Cell. Biol. 21:7307-7319). The association with hnRNP A2/B1 suggests that cyclin L may play a role in NF-kB-dependent regulation of mRNA processing or transport.

The confirmation that the expression of the Cyclin L polynucleotide and encoded peptide are inhibited by NFkB suggests that antagonists directed against the Cyclin L polynucleotide and/or encoded peptide would be useful for treating, diagnosing, and/or ameliorating disorders associated with aberrant NFkB activity, autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, HIV propagation in cells infected with other viruses, in addition to other NFkB associated diseases or disorders known in the art or described herein.

Moreover, antagonists directed against the Cyclin L polynucleotide and/or encoded peptide are useful for decreasing NF-kB activity, decreasing apoptotic events, and/or increasing IkBa expression or activity levels.

The Cyclin L NFkB associated polynucleotide and polypeptide of the present invention, including antagonists and/or fragments thereof, have uses that include detecting, prognosing, treating, preventing, and/or ameliorating the following diseases and/or disorders: immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis, inflammatory bowel disease, colitis, asthma, atherosclerosis, cachexia, euthyroid sick syndrome, stroke, and EAE.

The Cyclin L NFkB associated polynucleotide and polypeptide of the present invention, including antagonists and/or fragments thereof, have uses that include modulating the phosphorylation of IkB, modulate the activity of IKK-1, IKK-2, IKK-γ, modulate developmental processes, modulate epidermal differentiation, modulate the activity and/or expression levels of various cytokines, cytokine receptors, chemokines, adhesion molecules, acute phase proteins, anti-apoptotic proteins, and enzymes including iNOS and COX-2. Representative examples of cytokines, chemokines, cytokine receptors, and anti-apoptotic proteins are provided elsewhere herein or are otherwise known in the art (e.g., see as described herein).

In preferred embodiments, the following N-terminal Cyclin L deletion polypeptides are encompassed by the present invention: M1-R526, A2-R526, S3-R526, G4-R526, P5-R526, H6-R526, S7-R526, T8-R526, A9-R526, T10-R526, A11-R526, A12-R526, A13-R526, A14-R526, A15-R526, S16-R526, S17-R526, A18-R526, A19-R526, P20-R526, S21-R526, A22-R526, G23-R526, G24-R526, S25-R526, S26-R526, S27-R526, G28-R526, T29-R526, T30-R526, T31-R526, T32-R526, T33-R526, T34-R526, T35-R526, T36-R526, T37-R526, G38-R526, G39-R526, I40-R526, L41-R526, I42-R526, G43-R526, D44-R526, R45-R526, L46-R526, Y47-R526, S48-R526, E49-R526, V50-R526, S51-R526, L52-R526, T53-R526, I54-R526, D55-R526, H56-R526, S57-R526, L58-R526, I59-R526, P60-R526, E61-R526, E62-R526, R63-R526, L64-R526, S65-R526, P66-R526, T67-R526, P68-R526, S69, R526, M70-R526, Q71-R526, D72-R526, G73-R526, L74-R526, D75-R526, L76-R526, P77-R526, S78-R526, E79-R526, T80-R526, D81-R526, L82-R526, R83-R526, I84-R526, L85-R526, G86-R526, C87-R526, E88-R526, L89-R526, I90-R526, Q91-R526, A92-R526, A93-R526, G94-R526, I95-R526, L96-R526, L97-R526, R98-R526, L99-R526, P100-R526, Q101-R526, V102-R526, A103-R526, M104-R526, A105-R526, T106-R526, G107-R526, Q108-R526, V109-R526, L110-R526, F111-R526, H112-R526, R113-R526, F114-R526, F115-R526, Y116-R526, S117-R526, K118-R526, S119-R526, F120-R526, V121-R526, K122-R526, H123-R526, S124-R526, F125-R526, E126-R526, I117-R526, V128-R526, A129-R526, M130-R526, A131-R526, C132-R526, I133-R526, N134-R526, L135-R526, A136-R526, S137-R526, K138-R526, I139-R526, E140-R526, E141-R526, A142-R526, P143-R526, R144-R526, R145-R526, I146-R526, R147-R526, D148-R526, V149-R526, I150-R526, N151-R526, V152-R526, F153-R526, H154-R526, H155-R526, L156-R526, R157-R526, Q158-R526, L159-R526, R160-R526, G161-R526, K162-R526, R163-R526, T164-R526, P165-R526, S166-R526, P167-R526, L168-R526, I169-R526, L170-R526, D171-R526, Q172-R526, N173-R526, Y174-R526, I175-R526, N176-R526, T177-R526, K178-R526, N179-R526, Q180-R526, V181-R526, I182-R526, K183-R526, A184-R526, E185-R526, R186-R526, R187-R526, V188-R526, L189-R526, K190-R526, E191-R526, L192-R526, G193-R526, F194-R526, C195-R526, V196-R526, H197-R526, V198-R526, K199-R526, H200-R526, P201-R526, H202-R526, K203-R526, I204-R526, I205-R526, V206-R526, M207-R526, Y208-R526, L209-R526, Q210-R526, V211-R526, L212-R526, E213-R526, C214-R526, E215-R526, R216-R526, N217-R526, Q218-R526, T219-R526, L220-R526, V221-R526, Q222-R526, T223-R526, A224-R526, W225-R526, N226-R526, Y227-R526, M228-R526, N229-R526, D230-R526, S231-R526, L232-R526, R233-R526, T234-R526, N235-R526, V236-R526, F237-R526, V238-R526, R239-R526, F240-R526, Q241-R526, P242-R526, E243-R526, T244-R526, I245-R526, A246-R526, C247-R526, A248-R526, C249-R526, I250-R526, Y251-R526, L252-R526, A253-R526, A254-R526, R255-R526, A256-R526, L257-R526, Q258-R526, I259-R526, P260-R526, L261-R526, P262-R526, T263-R526, R264-R526, P265-R526, H266-R526, W267-R526, F268-R526, L269-R526, L270-R526, F271-R526, G272-R526, T273-R526, T274-R526, E275-R526, E276-R526, E277-R526, I278-R526, Q279-R526, E280-R526, I281-R526, C282-R526, I283-R526, E284-R526, T285-R526, L286-R526, R287-R526, L288-R526, Y289-R526, T290-R526, R291-R526, K292-R526, K293-R526, P294-R526, N295-R526, Y296-R526, E297-R526, L298-R526, L299-R526, E300-R526, K301-R526, E302-R526, V303-R526, E304-R526, K305-R526, R306-R526, K307-R526, V308-R526, A309-R526, L310-R526, Q311-R526, E312-R526, A313-R526, K314-R526, L315-R526, K316-R526, A317-R526, K318-R526, G319-R526, L320-R526, N321-R526, P322-R526, D323-R526, G324-R526, T325-R526, P326-R526, A327-R526, L328-R526, S329-R526, T330-R526, L331-R526, G332-R526, G333-R526, F334-R526, S335-R526, P336-R526, A337-R526, S338-R526, K339-R526, P340-R526, S341-R526, S342-R526, P343-R526, R344-R526, E345-R526, V346-R526, K347-R526, A348-R526, E349-R526, E350-R526, K351-R526, S352-R526, P353-R526, I354-R526, S355-R526, I356-R526, N357-R526, V358-R526, K359-R526, T360-R526, V361-R526, K362-R526, K363-R526, E364-R526, P365-R526, E366-R526, D367-R526, R368-R526, Q369-R526, Q370-R526, A371-R526, S372-R526, K373-R526, S374-R526, P375-R526, Y376-R526, N377-R526, G378-R526, V379-R526, R380-R526, K381-R526, D382-R526, S383-R526, K384-R526, R385-R526, S386-R526, R387-R526, N388-R526, S389-R526, R390-R526, S391-R526, A392-R526, S393-R526, R394-R526, S395-R526, R396-R526, S397-R526, R398-R526, T399-R526, R400-R526, S401-R526, R402-R526, S403-R526, R404-R526, S405-R526, H406-R526, T407-R526, P408-R526, R409-R526, R410-R526, H411-R526, Y412-R526, N413-R526, N414-R526, R415-R526, R416-R526, S417-R526, R418-R526, S419-R526, G420-R526, T421-R526, Y422-R526, S423-R526, S424-R526, R425-R526, S426-R526, R427-R526, S428-R526, R429-R526, S430-R526, R431-R526, S432-R526, H433-R526, S434-R526, E435-R526, S436-R526, P437-R526, R438-R526, R439-R526, H440-R526, H441-R526, N442-R526, H443-R526, G444-R526, S445-R526, P446-R526, H447-R526, L448-R526, K449-R526, A450-R526, K451-R526, H452-R526, T453-R526, R454-R526, D455-R526, D456-R526, L457-R526, K458-R526, S459-R526, S460-R526, N461-R526, R462-R526, H463-R526, G464-R526, H465-R526, K466-R526, R467-R526, K468-R526, K469-R526, S470-R526, R471-R526, S472-R526, R473-R526, S474-R526, Q475-R526, S476-R526, K477-R526, S478-R526, R479-R526, D480-R526, H481-R526, S482-R526, D483-R526, A484-R526, A485-R526, K486-R526, K487-R526, H488-R526, R489-R526, H490-R526, E491-R526, R492-R526, G493-R526, H494-R526, H495-R526, R496-R526, D497-R526, R498-R526, R499-R526, E500-R526, R501-R526, S502-R526, R503-R526, S504-R526, F505-R526, E506-R526, R507-R526, S508-R526, H509-R526, K510-R526, S511-R526, K512-R526, H513-R526, H514-R526, G515-R526, G516-R526, S517-R526, R518-R526, S519-R526, and/or G520-R526 of SEQ ID NO:128. Polynucleotide sequences encoding these polypeptides are also provided. The present invention also encompasses the use of these N-terminal Cyclin L deletion polypeptides as immunogenic and/or antigenic epitopes as described elsewhere herein.

In preferred embodiments, the following C-terminal Cyclin L deletion polypeptides are encompassed by the present invention: M1-R526, M1-R525, M1-H524, M1-R523, M1-G522, M1-H521, M1-G520, M1-S519, M1-R518, M1-S517, M1-G516, M1-G515, M1-H514, M1-H513, M1-K512, M1-S511, M1-K510, M1-H509, M1-S508, M1-R507, M1-E506, M1-F505, M1-S504, M1-R503, M1-S502, M1-R501, M1-E500, M1-R499, M1-R498, M1-D497, M1-R496, M1-H495, M1-H494, M1-G493, M1-R492, M1-E491, M1-H490, M1-R489, M1-H488, M1-K487, M1-K486, M1-A485, M1-A484, M1-D483, M1-S482, M1-H481, M1-D480, M1-R479, M1-S478, M1-K477, M1-S476, M1-Q475, M1-S474, M1-R473, M1-S472, M1-R471, M1-S470, M1-K469, M1-K468, M1-R467, M1-K466, M1-H465, M1-G464, M1-H463, M1-R462, M1-N461, M1-S460, M1-S459, M1-K458, M1-L457, M1-D456, M1-D455, M1-R454, M1-T453, M1-H452, M1-K451, M1-A450, M1-K449, M1-L448, M1-H447, M1-P446, M1-S445, M1-G444, M1-H443, M1-M1-H441, M1-H440, M1-R439, M1-R438, M1-P437, M1-S436, M1-E435, M1-M1-H433, M1-S432, M1-R431, M1-S430, M1-R429, M1-S428, M1-R427, M1-S434, M1-R425, M1-S424, M1-S423, M1-Y422, M1-T421, M1-G420, M1-S419, M1-R418, M1-S417, M1-R416, M1-R415, M1-N414, M1-N413, M1-Y412, M1-H411, M1-R410, M1-R409, M1-P408, M1-T407, M1-H406, M1-S405, M1-R404, M1-S403, M1-R402, M1-S401, M1-R400, M1-T399, M1-R398, M1-S397, M1-R396, M1-S395, M1-R394, M1-S393, M1-A392, M1-S391, M1-R390, M1-S389, M1-N388, M1-R387, M1-S386, M1-R385, M1-K384, M1-S383, M1-D382, M1-K381, M1-R380, M1-V379, M1-G378, M1-N377, M1-Y376, M1-P375, M1-S374, M1-K373, M1-S372, M1-A371, M1-Q370, M1-Q369, M1-R368, M1-D367, M1-E366, M1-P365, M1-E364, M1-K363, M1-K362, M1-V361, M1-T360, M1-K359, M1-V358, M1-N357, M1-1356, M1-S355, M1-1354, M1-P353, M1-S352, M1-K351, M1-E350, M1l-E349, M1-A348, M1-K347, M1-V346, M1-E345, M1-R344, M1-P343, M1-S342, M1-S341, M1-P340, M1-K339, M1-S338, M1-A337, M1-P336, M1-S335, M1-F334, M1-G333, M1-G332, M1-L331, M1-T330, M1-S329, M1-L328, M1-A327, M1-P326, M1-T325, M1-G324, M1-D323, M1-P322, M1-N321, M1-L320, M1-G319, M1-K318, M1-A317, M1-K316, M1-L315, M1-K314, M1-A313, M1-E312, M1-Q311, M1-L310, M1-A309, M1-V308, M1-K307, M1-R306, M1-K305, M1-E304, M1-V303, M1-E302, M1-K301, M1-E300, M1-L299, M1-L298, M1-E297, M1-Y296, M1-N295, M1-P294, M1-K293, M1-K292, M1-R291, M1-T290, M1-Y289, M1-L288, M1-R287, M1-L286, M1-T285, M1-E284, M1-I283, M1-C282, M1-I281, M1-E280, M1-Q279, M1-I278, M1-E277, M1-E276, M1-E275, M1-T274, M1-T273, M1-G272, M1-F271, M1-L270, M1-L269, M1-F268, M1-W267, M1-H266, M1-P265, M1-R264, M1-T263, M1-P262, M1-L261, M1-P260, M1-I259, M1-Q258, M1-L257, M1-A256, M1-R255, M1-A254, M1-A253, M1-L252, M1-Y251, M1-I250, M1-C249, M1-A248, M1-C247, M1-A246, M1-I245, M1-T244, M1-E243, M1-P242, M1-Q241, M1-F240, M1-R239, M1-V238, M1-F237, M1-V236, M1-N235, M1-T234, M1-R233, M1-L232, M1-S231, M1-D230, M1-N229, M1-M228, M1-Y227, M1-N226, M1-W225, M1-A224, M1-T223, M1-Q222, M1-V221, M1-L220, M1-T219, M1-Q218, M1-N217, M1-R216, M1-E215, M1-C214, M1-E213, M1-L212, M1-V211, M1-Q210, M1-L209, M1-Y208, M1-M207, M1-V206, M1-I205, M1-I204, M1-K203, M1-H202, M1-P201, M1-H200, M1-K199, M1-V198, M1-H197, M1-V196, M1-C195, M1-F194, M1-G193, M1-L192, M1-E191, M1-K190, M1-L189, M1-V188, M1-R187, M1-R186, M1-E185, M1-A184, M1-K183, M1-I182, M1-V181, M1-Q180, M1-N179, M1-K178, M1-T177, M1-N176, M1-I175, M1-Y174, M1-N173, M1-Q172, M1-D171, M1-L170, M1-I169, M1-L168, M1-P167, M1-S166, M1-P165, M1-T164, M1-R163, M1-K162, M1-G161, M1-R160, M1-L159, M1-Q158, M1-R157, M1-L156, M1-H155, M1-H154, M1-F153, M1-V152, M1-N151, M1-1150, M1-V149, M1-D148, M1-R147, M1-I146, M1-R145, M1-R144, M1-P143, M1-A142, M1-E141, M1-E410, M1-I139, M1-K138, M1-S137, M1-A136, M1-L135, M1-N134, M1-I133, M1-C132, M1-A131, M1-M130, M1-A129, M1-V128, M1-I127, M1-E126, M1-F125, M1-S124, M1-H123, M1-K122, M1-V121, M1-F120, M1-S119, M1-K118, M1-S117, M1-Y116, M1-F115, M1-F114, M1-R113, M1-H112, M1-F111, M1-L110, M1-V109, M1-Q108, M1-G107, M1-T106, M1-A105, M1-M104, M1-A103, M1-V102, M1-Q101, M1-P100, M1-L99, M1-R98, M1-L97, M1-L96, M1-I95, M1-G94, M1-A93, M1-A92, M1-Q91, M1-I90, M1-L89, M1-E88, M1-C87, M1-G86, M1-L85, M1-I84, M1-R83, M1-L82, M1-D81, M1-T80, M1-E79, M1-S78, M1-P77, M1-L76, M1-D75, M1-L74, M1-G73, M1-D72, M1-Q71, M1-M70, M1-S69, M1-P68, M1-T67, M1-P66, M1-S65, M1-L64, M1-R63, M1-E62, M1-E61, M1-P60, M1-159, M1-L58, M1-S57, M1-H56, M1-D55, M1-154, M1-T53, M1-L52, M1-S51, M1-V50, M1-E49, M1-S48, M1-Y47, M1-L46, M1-R45, M1-D44, M1-G43, M1-142, M1-L41, M1-I40, M1-G39, M1-G38, M1-T37, M1-T36, M1-T35, M1-T34, M1-T33, M1-T32, M1-T31, M1-T30, M1-T29, M1-G28, M1-S27, M1-S26, M1-S25, M1-G24, M1-G23, M1-A22, M1-S21, M1-P20, M1-A19, M1-A18, M1-S17, M1-S16, M1-A15, M1-A14, M1-A13, M1-A12, M1-A11, M1-T10, M1-A9, M1-T8, and/or M1-S7 of SEQ ID NO:128. Polynucleotide sequences encoding these polypeptides are also provided. The present invention also encompasses the use of these C-terminal Cyclin L deletion polypeptides as immunogenic and/or antigenic epitopes as described elsewhere herein.

Table I and III summarizes the information corresponding to each “Gene No.” described above. Unless otherwise described, the nucleotide sequence identified as “NT SEQ ID NO:1-108, 125, 127, 132-140, 158-159, or 264-284” was assembled from partially homologous (“overlapping”) sequences obtained from the “Clone Name” identified in Table I and III and, in some cases, from additional related DNA clones. The overlapping sequences were assembled into a single contiguous sequence of high redundancy (usually several overlapping sequences at each nucleotide position), resulting in a final sequence identified as SEQ ID NO:X.

“Total NT Seq. Of Clone” refers to the total number of nucleotides in the clone contig identified by “Gene No.” The nucleotide position of SEQ ID NO:X of the putative start codon (methionine) is identified as “5′ NT of Start Codon of ORF.”

The translated amino acid sequence, beginning with the methionine, is identified as “SEQ ID NO:Y” although other reading frames can also be easily translated using known molecular biology techniques. The polypeptides produced by these alternative open reading frames are specifically contemplated by the present invention.

The total number of amino acids within the open reading frame of SEQ ID NO:Y is identified as “Total AA of ORF”.

SEQ ID NO:X (where X may be any of the polynucleotide sequences disclosed in the sequence listing) and the translated SEQ ID NO:Y (where Y may be any of the polypeptide sequences disclosed in the sequence listing) are sufficiently accurate and otherwise suitable for a variety of uses well known in the art and described further herein. For instance, SEQ ID NO:1-108, 125, 127, 132-140, 158-159, or 264-284 is useful for designing nucleic acid hybridization probes that will detect nucleic acid sequences contained in SEQ ID NO:1-108, 125, 127, 132-140, 158-159, or 264-284. These probes will also hybridize to nucleic acid molecules in biological samples, thereby enabling a variety of forensic and diagnostic methods of the invention. Similarly, polypeptides identified from 109-118, 126, 128, 144-152, or 160-161 may be used, for example, to generate antibodies which bind specifically to proteins containing the polypeptides and the proteins encoded by the cDNA clones identified in Table I and III.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides may cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1-108, 125, 127, 132-140, 158-159, or 264-284 and the predicted translated amino acid sequence identified as 109-118, 126, 128, 144-152, or 160-161. The nucleotide sequence of each clone can readily be determined by sequencing the clone in accordance with known methods. The predicted amino acid sequence can then be verified from such clones. Moreover, the amino acid sequence of the protein encoded by a particular clone can also be directly determined by peptide sequencing or by expressing the protein in a suitable host cell containing the cDNA, collecting the protein, and determining its sequence.

The present invention also relates to the genes corresponding to SEQ ID NO:1-108, 125, 127, 132-140, 158-159, or 264-284, 109-118, 126, 128, 144-152, or 160-161. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologs, allelic variants, and/or orthologs. The skilled artisan could, using procedures well-known in the art, obtain the polynucleotide sequence corresponding to full-length genes (including, but not limited to the full-length coding region), allelic variants, splice variants, orthologs, and/or species homologues of genes corresponding to SEQ ID NO:1-108, 125, 127, 132-140, 158-159, or 264-284, 109-118, 126, 128, 144-152, or 160-161. For example, allelic variants and/or species homologues may be isolated and identified by making suitable probes or primers which correspond to the 5′, 3′, or internal regions of the sequences provided herein and screening a suitable nucleic acid source for allelic variants and/or the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the protein, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using protocols described herein or otherwise known in the art, such as, for example, antibodies of the invention raised against the full-length form of the protein.

The present invention provides a polynucleotide comprising, or alternatively consisting of, the sequence identified as SEQ ID NO:1-108, 125, 127, 132-140, 158-159, or 264-284. The present invention also provides a polypeptide comprising, or alternatively consisting of, the sequence identified as 109-118, 126, 128, 144-152, or 160-161. The present invention also provides polynucleotides encoding a polypeptide comprising, or alternatively consisting of the polypeptide sequence of 109-118, 126, 128, 144-152, or 160-161.

Preferably, the present invention is directed to a polynucleotide comprising, or alternatively consisting of, the sequence identified as SEQ ID NO:1-108, 125, 127, 132-140, 158-159, or 264-284 that is less than, or equal to, a polynucleotide sequence that is 5 mega basepairs, 1 mega basepairs, 0.5 mega basepairs, 0.1 mega basepairs, 50,000 basepairs, 20,000 basepairs, or 10,000 basepairs in length.

The present invention encompasses polynucleotides with sequences complementary to those of the polynucleotides of the present invention disclosed herein. Such sequences may be complementary to the sequence disclosed as SEQ ID NO:1-108, 125, 127, 132-140, 158-159, or 264-284, and/or the nucleic acid sequence encoding the sequence disclosed as 109-118, 126, 128, 144-152, or 160-161.

The present invention also encompasses polynucleotides capable of hybridizing, preferably under reduced stringency conditions, more preferably under stringent conditions, and most preferably under highly stringent conditions, to polynucleotides described herein. Examples of stringency conditions are shown in Table VI below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE VI

| | Poly- | Hybrid | Hybridization | Wash |
|---|---|---|---|---|
| Stringency | nucleotide | Length | Temperature | Temperature |
| Condition | Hybrid ± | (bp)‡ | and Buffer† | and Buffer† |
| A | DNA:DNA | > or equal | 65° C.; 1xSSC- | 65° C.; 0.3xSSC |
| | | to 50 | or −42° C.; | |
| | | | 1xSSC, 50% | |
| | | | formamide | |
| B | DNA:DNA | <50 | Tb*; 1xSSC | Tb*; 1xSSC |
| C | DNA:RNA | > or equal | 67° C.; 1xSSC- | 67° C.; 0.3xSSC |
| | | to 50 | or −45° C.; | |
| | | | 1xSSC, 50% | |
| | | | formamide | |
| D | DNA:RNA | <50 | Td*; 1xSSC | Td*; 1xSSC |
| E | RNA:RNA | > or equal | 70° C.; 1xSSC- | 70° C.; 0.3xSSC |
| | | to 50 | or −50° C.; | |
| | | | 1xSSC, 50% | |
| | | | formamide | |
| F | RNA:RNA | <50 | Tf*; 1xSSC | Tf*; 1xSSC |
| G | DNA:DNA | > or equal | 65° C.; 4xSSC- | 65° C.; 1xSSC |
| | | to 50 | or −45° C.; | |
| | | | 4xSSC, 50% | |
| | | | formamide | |
| H | DNA:DNA | <50 | Th*; 4xSSC | Th*; 4xSSC |
| I | DNA:RNA | > or equal | 67° C.; 4xSSC- | 67° C.; 1xSSC |
| | | to 50 | or −45° C.; | |
| | | | 4xSSC, 50% | |
| | | | formamide | |
| J | DNA:RNA | <50 | Tj*; 4xSSC | Tj*; 4xSSC |
| K | RNA:RNA | > or equal | 70° C.; 4xSSC- | 67° C.; 1xSSC |
| | | to 50 | or −40° C.; | |
| | | | 6xSSC, 50% | |
| | | | formamide | |
| L | RNA:RNA | <50 | Tl*; 2xSSC | Tl*; 2xSSC |
| M | DNA:DNA | > or equal | 50° C.; 4xSSC- | 50° C.; 2xSSC |
| | | to 50 | or −40° C. | |
| | | | 6xSSC, 50% | |
| | | | formamide | |
| N | DNA:DNA | <50 | Tn*; 6xSSC | Tn*; 6xSSC |
| O | DNA:RNA | > or equal | 55° C.; 4xSSC- | 55° C.; 2xSSC |
| | | to 50 | or −42° C.; | |
| | | | 6xSSC, 50% | |
| | | | formamide | |
| P | DNA:RNA | <50 | Tp*; 6xSSC | Tp*; 6xSSC |
| Q | RNA:RNA | > or equal | 60° C.; 4xSSC- | 60° C.; 2xSSC |
| | | to 50 | or −45° C.; | |
| | | | 6xSSC, 50% | |
| | | | formamide | |
| R | RNA:RNA | <50 | Tr*; 4xSSC | Tr*; 4xSSC |

‡—The “hybrid length” is the anticipated length for the hybridized region(s) of the hybridizing polynucleotides. When hybridizing a polynucleotide of unknown sequence, the hybrid is assumed to be that of the hybridizing polynucleotide of the present invention. When polynucleotides of known sequence are hybridized, the hybrid length can be determined by aligning the sequences of the polynucleotides and identifying the region or regions of optimal sequence complementarity. Methods of aligning two or more polynucleotide sequences and/or determining the percent identity between two polynucleotide sequences are well known in the art (e.g., MegAlign program of the DNA*Star suite of programs, etc).

†—SSPE (1×SSPE is 0.15M NaCl, 10 mM NaH2PO4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. The hydridizations and washes may additionally include 5× Denhardt's reagent, 0.5-1.0% SDS, 100 ug/ml denatured, fragmented salmon sperm DNA, 0.5% sodium pyrophosphate, and up to 50% formamide.

*Tb-Tr: The hybridization temperature for hybrids anticipated to be less than 50 base pairs in length should be 5-10° C. less than the melting temperature Tm of the hybrids there Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log₁₀[Na+])+0.41(% G+C)−(600/N), where N is the number of bases in the hybrid, and [Na+] is the concentration of sodium ions in the hybridization buffer ([NA+] for 1×SSC=0.165 M).

±—The present invention encompasses the substitution of any one, or more DNA or RNA hybrid partners with either a PNA, or a modified polynucleotide. Such modified polynucleotides are known in the art and are more particularly described elsewhere herein.

Additional examples of stringency conditions for polynucleotide hybridization are provided, for example, in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M., Ausubel et al., eds, John Wiley and Sons, Inc., sections 2.10 and 6.3-6.4, which are hereby incorporated by reference herein.

Preferably, such hybridizing polynucleotides have at least 70% sequence identity (more preferably, at least 80% identity; and most preferably at least 90% or 95% identity) with the polynucleotide of the present invention to which they hybridize, where sequence identity is determined by comparing the sequences of the hybridizing polynucleotides when aligned so as to maximize overlap and identity while minimizing sequence gaps. The determination of identity is well known in the art, and discussed more specifically elsewhere herein.

The invention encompasses the application of PCR methodology to the polynucleotide sequences of the present invention, and/or the cDNA encoding the polypeptides of the present invention. PCR techniques for the amplification of nucleic acids are described in U.S. Pat. No. 4, 683, 195 and Saiki et al., Science, 239:487-491 (1988). PCR, for example, may include the following steps, of denaturation of template nucleic acid (if double-stranded), annealing of primer to target, and polymerization. The nucleic acid probed or used as a template in the amplification reaction may be genomic DNA, cDNA, RNA, or a PNA. PCR may be used to amplify specific sequences from genomic DNA, specific RNA sequence, and/or cDNA transcribed from mRNA. References for the general use of PCR techniques, including specific method parameters, include Mullis et al., Cold Spring Harbor Symp. Quant. Biol., 51:263, (1987), Ehrlich (ed), PCR Technology, Stockton Press, N.Y., 1989; Ehrlich et al., Science, 252:1643-1650, (1991); and “PCR Protocols, A Guide to Methods and Applications”, Eds., Innis et al., Academic Press, New York, (1990).

### Signal Sequences

The present invention also encompasses mature forms of the polypeptide comprising, or alternatively consisting of, the polypeptide sequence of 109-118, 126, 128, 144-152, or 160-161, the polypeptide encoded by the polynucleotide described as SEQ ID NO:1-108, 125, 127, 132-140, 158-159, or 264-284. The present invention also encompasses polynucleotides encoding mature forms of the present invention, such as, for example the polynucleotide sequence of SEQ ID NO:1-108, 125, 127, 132-140, 158-159, or 264-284.

According to the signal hypothesis, proteins secreted by eukaryotic cells have a signal or secretary leader sequence which is cleaved from the mature protein once export of the growing protein chain across the rough endoplasmic reticulum has been initiated. Most eukaryotic cells cleave secreted proteins with the same specificity. However, in some cases, cleavage of a secreted protein is not entirely uniform, which results in two or more mature species of the protein. Further, it has long been known that cleavage specificity of a secreted protein is ultimately determined by the primary structure of the complete protein, that is, it is inherent in the amino acid sequence of the polypeptide.

Methods for predicting whether a protein has a signal sequence, as well as the cleavage point for that sequence, are available. For instance, the method of McGeoch, Virus Res. 3:271-286 (1985), uses the information from a short N-terminal charged region and a subsequent uncharged region of the complete (uncleaved) protein. The method of von Heinje, Nucleic Acids Res. 14:4683-4690 (1986) uses the information from the residues surrounding the cleavage site, typically residues -13 to +2, where +1 indicates the amino terminus of the secreted protein. The accuracy of predicting the cleavage points of known mammalian secretory proteins for each of these methods is in the range of 75-80%. (von Heinje, supra.) However, the two methods do not always produce the same predicted cleavage point(s) for a given protein.

The established method for identifying the location of signal sequences, in addition, to their cleavage sites has been the SignalP program (v1.1) developed by Henrik Nielsen et al., Protein Engineering 10:1-6 (1997). The program relies upon the algorithm developed by von Heinje, though provides additional parameters to increase the prediction accuracy.

More recently, a hidden Markov model has been developed (H. Neilson, et al., Ismb 1998;6:122-30), which has been incorporated into the more recent SignalP (v2.0). This new method increases the ability to identify the cleavage site by discriminating between signal peptides and uncleaved signal anchors. The present invention encompasses the application of the method disclosed therein to the prediction of the signal peptide location, including the cleavage site, to any of the polypeptide sequences of the present invention.

As one of ordinary skill would appreciate, however, cleavage sites sometimes vary from organism to organism and cannot be predicted with absolute certainty. Accordingly, the polypeptide of the present invention may contain a signal sequence. Polypeptides of the invention which comprise a signal sequence have an N-terminus beginning within 5 residues (i.e., + or −5 residues, or preferably at the −5, −4, −3, −2, −1, +1, +2, +3, +4, or +5 residue) of the predicted cleavage point. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. Nonetheless, the present invention provides the mature protein produced by expression of the polynucleotide sequence of SEQ ID NO:1-108, 125, 127, 132-140, 158-159, or 264-284 in a mammalian cell (e.g., COS cells, as described below). These polypeptides, and the polynucleotides encoding such polypeptides, are contemplated by the present invention.

### Polynucleotide and Polypeptide Variants

The present invention also encompasses variants (e.g., allelic variants, orthologs, etc.) of the polynucleotide sequence disclosed herein in SEQ ID NO:1-108, 125, 127, 132-140, 158-159, or 264-284, and/or the complementary strand thereto.

The present invention also encompasses variants of the polypeptide sequence, and/or fragments therein, disclosed in 109-118, 126, 128, 144-152, or 160-161, a polypeptide encoded by the polynucleotide sequence in SEQ ID NO:1-108, 125, 127, 132-140, 158-159, or 264-284.

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

Thus, one aspect of the invention provides an isolated nucleic acid molecule comprising, or alternatively consisting of, a polynucleotide having a nucleotide sequence selected from the group consisting of: (a) a nucleotide sequence encoding a NFKB related polypeptide having an amino acid sequence as shown in the sequence listing and described in SEQ ID NO:1-108, 125, 127, 132-140, 158-159, or 264-284; (b) a nucleotide sequence encoding a mature NFKB related polypeptide having the amino acid sequence as shown in the sequence listing and described in SEQ ID NO:1-108, 125, 127, 132-140, 158-159, or 264-284; (c) a nucleotide sequence encoding a biologically active fragment of a NFKB related polypeptide having an amino acid sequence shown in the sequence listing and described in SEQ ID NO:1-108, 125, 127, 132-140, 158-159, or 264-284; (d) a nucleotide sequence encoding an antigenic fragment of a NFKB related polypeptide having an amino acid sequence sown in the sequence listing and described in SEQ ID NO:1-108, 125, 127, 132-140, 158-159, or 264-284; (e) a nucleotide sequence encoding a NFKB related polypeptide comprising the complete amino acid sequence encoded by a human cDNA plasmid contained in SEQ ID NO:1-108, 125, 127, 132-140, 158-159, or 264-284; (f) a nucleotide sequence encoding a mature NFKB related polypeptide having an amino acid sequence encoded by a human cDNA plasmid contained in SEQ ID NO:1-108, 125, 127, 132-140, 158-159, or 264-284; (g) a nucleotide sequence encoding a biologically active fragment of a NFKB related polypeptide having an amino acid sequence encoded by a human cDNA plasmid contained in SEQ ID NO:1-108, 125, 127, 132-140, 158-159, or 264-284; (h) a nucleotide sequence encoding an antigenic fragment of a NFKB related polypeptide having an amino acid sequence encoded by a human cDNA plasmid contained in SEQ ID NO:1-108, 125, 127, 132-140, 158-159, or 264-284; (i) a nucleotide sequence complimentary to any of the nucleotide sequences in (a), (b), (c), (d), (e), (f), (g), or (h), above.

The present invention is also directed to polynucleotide sequences which comprise, or alternatively consist of, a polynucleotide sequence which is at least about 80%, 85%, 90%, 91%, 92%, 93%, 94%, 95%, 96%, 97%, 98%, 99%, 99.1% 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

Another aspect of the invention provides an isolated nucleic acid molecule comprising, or alternatively, consisting of, a polynucleotide having a nucleotide sequence selected from the group consisting of: (a) a nucleotide sequence encoding a NFKB related polypeptide having an amino acid sequence as shown in the sequence listing and descried in Table I and III; (b) a nucleotide sequence encoding a mature NFKB related polypeptide having the amino acid sequence as shown in the sequence listing and descried in Table I and III; (c) a nucleotide sequence encoding a biologically active fragment of a NFKB related polypeptide having an amino acid sequence as shown in the sequence listing and descried in Table I and III; (d) a nucleotide sequence encoding an antigenic fragment of a NFKB related polypeptide having an amino acid sequence as shown in the sequence listing and descried in Table I and III; (e) a nucleotide sequence encoding a NFKB related polypeptide comprising the complete amino acid sequence encoded by a human cDNA described in Table I and III; (f) a nucleotide sequence encoding a mature NFKB related polypeptide having an amino acid sequence encoded by a human cDNA described in Table I and III: (g) a nucleotide sequence encoding a biologically active fragment of a NFKB related polypeptide having an amino acid sequence encoded by a human cDNA described in Table I and III; (h) a nucleotide sequence encoding an antigenic fragment of a NFKB related polypeptide having an amino acid sequence encoded by a human cDNA in a cDNA plasmid described in Table I and III; (i) a nucleotide sequence complimentary to any of the nucleotide sequences in (a), (b), (c), (d), (e), (f), (g), or (h) above.

The present invention is also directed to nucleic acid molecules which comprise, or alternatively, consist of, a nucleotide sequence which is at least about 80%, 85%, 90%, 91%, 92%, 93%, 94%, 95%, 96%, 97%, 98%, 99%, 99.1% 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above.

The present invention encompasses polypeptide sequences which comprise, or alternatively consist of, an amino acid sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, the following non-limited examples, the polypeptide sequence identified as 109-118, 126, 128, 144-152, or 160-161, and/or polypeptide fragments of any of the polypeptides provided herein. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

The present invention is also directed to polypeptides which comprise, or alternatively consist of, an amino acid sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, the polypeptide sequence shown in 109-118, 126, 128, 144-152, or 160-161, a polypeptide sequence encoded by the nucleotide sequence in SEQ ID NO:1-108, 125, 127, 132-140, 158-159, or 264-284, a polypeptide sequence encoded by the cDNA in cDNA plasmid:Z, and/or polypeptide fragments of any of these polypeptides (e.g., those fragments described herein). Polynucleotides which hybridize to the complement of the nucleic acid molecules encoding these polypeptides under stringent hybridization conditions or alternatively, under lower stringency conditions, are also encompasses by the present invention, as are the polypeptides encoded by these polynucleotides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence referenced in Table I and III, the ORF (open reading frame), or any fragment specified as described herein.

As a practical matter, whether any particular nucleic acid molecule or polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced in Table 1 (SEQ ID NO:2) can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both amino acid sequences. The result of said global sequence alignment is in percent identity. Preferred parameters used in a CLUSTALW alignment of DNA sequences to calculate percent identity via pairwise alignments are: Matrix=BLOSUM, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global polypeptide alignments are often preferred since they reflect the percent identity between the polypeptide molecules as a whole (i.e., including any polypeptide overhangs, not just overlapping regions), as opposed to, only local matching polypeptides. Manual corrections for global percent identity determinations are required since the CLUSTALW program does not account for N- and C-terminal truncations of the subject sequence when calculating percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more polynucleotide or polypeptide sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modifed CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the mRNA to those preferred by a bacterial host such as E. coli).

Naturally occurring variants are called “allelic variants” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)).

Moreover, ample evidence demonstrates that variants often retain a biological activity similar to that of the naturally occurring protein. For example, Gayle and coworkers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the protein will likely be retained when less than the majority of the residues of the protein are removed from the N-terminus or C-terminus. Whether a particular polypeptide lacking N- or C-terminal residues of a protein retains such immunogenic activities can readily be determined by routine methods described herein and otherwise known in the art.

Alternatively, such N-terminus or C-terminus deletions of a polypeptide of the present invention may, in fact, result in a significant increase in one or more of the biological activities of the polypeptide(s). For example, biological activity of many polypeptides are governed by the presence of regulatory domains at either one or both termini. Such regulatory domains effectively inhibit the biological activity of such polypeptides in lieu of an activation event (e.g., binding to a cognate ligand or receptor, phosphorylation, proteolytic processing, etc.). Thus, by eliminating the regulatory domain of a polypeptide, the polypeptide may effectively be rendered biologically active in the absence of an activation event.

Thus, the invention further includes polypeptide variants that show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved.

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1990).

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1990).

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in Table VII below.

TABLE VII

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., β or γ amino acids.

Both identity and similarity can be readily calculated by reference to the following publications: Computational Molecular Biology, Lesk, A. M., ed., Oxford University Press, New York, 1988; Biocomputing: Informatics and Genome Projects, Smith, D. W., ed., Academic Press, New York, 1993; Informatics Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991.

In addition, the present invention also encompasses substitution of amino acids based upon the probability of an amino acid substitution resulting in conservation of function. Such probabilities are determined by aligning multiple genes with related function and assessing the relative penalty of each substitution to proper gene function. Such probabilities are often described in a matrix and are used by some algorithms (e.g., BLAST, CLUSTALW, GAP, etc.) in calculating percent similarity wherein similarity refers to the degree by which one amino acid may substitute for another amino acid without lose of function. An example of such a matrix is the PAM250 or BLOSUM62 matrix.

Aside from the canonical chemically conservative substitutions referenced above, the invention also encompasses substitutions which are typically not classified as conservative, but that may be chemically conservative under certain circumstances. Analysis of enzymatic catalysis for proteases, for example, has shown that certain amino acids within the active site of some enzymes may have highly perturbed pKa's due to the unique microenvironment of the active site. Such perturbed pKa's could enable some amino acids to substitute for other amino acids while conserving enzymatic structure and function. Examples of amino acids that are known to have amino acids with perturbed pKa's are the Glu-35 residue of Lysozyme, the Ile-16 residue of Chymotrypsin, the His-159 residue of Papain, etc. The conservation of function relates to either anomalous protonation or anomalous deprotonation of such amino acids, relative to their canonical, non-perturbed pKa. The pKa perturbation may enable these amino acids to actively participate in general acid-base catalysis due to the unique ionization environment within the enzyme active site. Thus, substituting an amino acid capable of serving as either a general acid or general base within the microenvironment of an enzyme active site or cavity, as may be the case, in the same or similar capacity as the wild-type amino acid, would effectively serve as a conservative amino substitution.

Besides conservative amino acid substitution, variants of the present invention include, but are not limited to, the following: (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as, for example, an IgG Fc fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

Moreover, the invention further includes polypeptide variants created through the application of molecular evolution (“DNA Shuffling”) methodology to the polynucleotide disclosed as SEQ ID NO:1-108, 125, 127, 132-140, 158-159, or 264-284, and/or the cDNA encoding the polypeptide disclosed as 109-118, 126, 128, 144-152, or 160-161. Such DNA Shuffling technology is known in the art and more particularly described elsewhere herein (e.g., WPC, Stemmer, PNAS, 91:10747, (1994)), and in the Examples provided herein).

A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5, 5-10, 5-25, 5-50, 10-50 or 50-150, conservative amino acid substitutions are preferable.

### Polynucleotide and Polypeptide Fragments

The present invention is directed to polynucleotide fragments of the polynucleotides of the invention, in addition to polypeptides encoded therein by said polynucleotides and/or fragments.

In the present invention, a “polynucleotide fragment” refers to a short polynucleotide having a nucleic acid sequence which: is a portion of that shown in SEQ ID NO:1-108, 125, 127, 132-140, 158-159, or 264-284 or the complementary strand thereto, or is a portion of a polynucleotide sequence encoding the polypeptide of 109-118, 126, 128, 144-152, or 160-161. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length” for example, is intended to include 20 or more contiguous bases from the nucleotide sequence shown in SEQ ID NO:1-108, 125, 127, 132-140, 158-159, or 264-284. In this context “about” includes the particularly recited value, a value larger or smaller by several (5, 4, 3, 2, or 1) nucleotides, at either terminus, or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:1-108, 125, 127, 132-140, 158-159, or 264-284, or the complementary strand thereto. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Also encompassed by the present invention are polynucleotides which hybridize to these nucleic acid molecules under stringent hybridization conditions or lower stringency conditions, as are the polypeptides encoded by these polynucleotides.

In the present invention, a “polypeptide fragment” refers to an amino acid sequence which is a portion of that contained in 109-118, 126, 128, 144-152, or 160-161. Protein (polypeptide) fragments may be “free-standing” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Preferred polypeptide fragments include the full-length protein. Further preferred polypeptide fragments include the full-length protein having a continuous series of deleted residues from the amino or the carboxy terminus, or both. For example, any number of amino acids, ranging from 1-60, can be deleted from the amino terminus of the full-length polypeptide. Similarly, any number of amino acids, ranging from 1-30, can be deleted from the carboxy terminus of the full-length protein. Furthermore, any combination of the above amino and carboxy terminus deletions are preferred. Similarly, polynucleotides encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of 109-118, 126, 128, 144-152, or 160-161 falling within conserved domains are specifically contemplated by the present invention. Moreover, polynucleotides encoding these domains are also contemplated.

Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity. Polynucleotides encoding these polypeptide fragments are also encompassed by the invention.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

The present invention encompasses polypeptides comprising, or alternatively consisting of, an epitope of the polypeptide having an amino acid sequence of 109-118, 126, 128, 144-152, or 160-161, or encoded by a polynucleotide that hybridizes to the complement of the sequence of SEQ ID NO:1-108, 125, 127, 132-140, 158-159, or 264-284 under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO: 1), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least I1, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length, or longer. Additional non-exclusive preferred antigenic epitopes include the antigenic epitopes disclosed herein, as well as portions thereof. Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of polynucleotides corresponding to SEQ ID NO:1-108, 125, 127, 132-140, 158-159, or 264-284 and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

### Antibodies

Further polypeptides of the invention relate to antibodies and T-cell antigen receptors (TCR) which immunospecifically bind a polypeptide, polypeptide fragment, or variant of 109-118, 126, 128, 144-152, or 160-161, and/or an epitope, of the present invention (as determined by immunoassays well known in the art for assaying specific antibody-antigen binding). Antibodies of the invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti--Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F(ab′)2 fragments) which are capable of specifically binding to protein. Fab and F(ab′)2 fragments lack the Fc fragment of intact antibody, clear more rapidly from the circulation of the animal or plant, and may have less non-specific tissue binding than an intact antibody (Wahl et al., J. Nucl. Med. . . 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10-2 M, 10-2 M, 5×10-3 M, 10-3 M, 5×10-4 M, 10-4 M, 5×10-5 M, 5×10 -6 M, 10-6M, 5×10-7 M, 107 M, 5×10-8 M, 10-8 M, 5×10-9 M, 10-9 M, 5×10 -10 M, 10-10 M, 5×10-11 M, 10-11 M, 5×10-12 M, 10-12 M, 5×10-13 M, 10-13 M, 5×10-14 M, 10-14 M, 5×10-15 M, or 10-15 M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85 %, at least 80%, at least 75%, at least 70%, at least 60%, or at least 50%.

Antibodies of the present invention may act as agonists or antagonists of the polypeptides of the present invention. For example, the present invention includes antibodies which disrupt the receptorAigand interactions with the polypeptides of the invention either partially or fully. Preferably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9): 1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art.

The antibodies of the present invention may comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988); and Current Protocols, Chapter 2; which are hereby incorporated herein by reference in its entirety). In a preferred method, a preparation of the NF-kB-associated polypeptides protein is prepared and purified to render it substantially free of natural contaminants. Such a preparation is then introduced into an animal in order to produce polyclonal antisera of greater specific activity. For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd} ed. (1988), by Hammerling, et al., Monoclonal Antibodies and T-Cell Hybridomas (Elsevier, N.Y., pp. 563-681 (1981); Köhler et al., Eur. J. Immunol. 6:511 (1976); Köhler et al., Eur. J. Immunol. 6:292 (1976), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include polypeptides of the present invention or a fusion protein thereof. Preferably, the immunizing agent consists of an NF-kB-associated polypeptides polypeptide or, more preferably, with a NF-KB-associated polypeptides polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C), and supplemented with about 10 g/l of nonessential amino acids, about 1,000 U/ml of penicillin, and about 100 ug/ml of streptomycin. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. More preferred are the parent myeloma cell line (SP2O) as provided by the ATCC. As inferred throughout the specification, human myeloma and mouse-human heteromycloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoabsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra, and/or according to Wands et al. (Gastroenterology 80:225-232 (1981)). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples described herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; Cabilly et al., Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985); U.S. Pat. Nos. 5,807,715; 4,816,567; and 4,816,397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988)1 and Presta, Curr. Op. Struct. Biol., 2:593-596 (1992).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147(1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Such anti-idiotypic antibodies capable of binding to the NF-kB-associated polypeptides polypeptide can be produced in a two-step procedure. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

The antibodies of the present invention may be bispecific antibodies. Bispecific antibodies are monoclonal, Preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of 109-118, 126, 128, 144-152, or 160-161.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeler et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, N.Y., which are both incorporated by reference herein in their entireties ), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

More preferably, a clone encoding an antibody of the present invention may be obtained according to the method described in the Example section herein.

### Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N.Y. (1993); Kriegler, Gene Transfer and Expression, A Laboratory Manual, Stockton Press, N.Y. (1990); and in Chapters 12 and 13, Dracopoli et al. (eds), Current Protocols in Human Genetics, John Wiley & Sons, N.Y. (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of 109-118, 126, 128, 144-152, or 160-161 may be fused or conjugated to the above antibody portions to increase the in vivo half life of the polypeptides or for use in immunoassays using methods known in the art. Further, the polypeptides corresponding to 109-118, 126, 128, 144-152, or 160-161 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fe portion may hinder therapy and diagnosis if the fusion protein is used as an antigen for immunizations. In drug discovery, for example, human proteins, such as hIL-5, have been fused with Fe portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

The present invention also encompasses the creation of synthetic antibodies directed against the polypeptides of the present invention. One example of synthetic antibodies is described in Radrizzani, M., et al., Medicina, (Aires), 59(6):753-8, (1999)). Recently, a new class of synthetic antibodies has been described and are referred to as molecularly imprinted polymers (MIPs) (Semorex, Inc.). Antibodies, peptides, and enzymes are often used as molecular recognition elements in chemical and biological sensors. However, their lack of stability and signal transduction mechanisms limits their use as sensing devices. Molecularly imprinted polymers (MIPs) are capable of mimicking the function of biological receptors but with less stability constraints. Such polymers provide high sensitivity and selectivity while maintaining excellent thermal and mechanical stability. MIPs have the ability to bind to small molecules and to target molecules such as organics and proteins' with equal or greater potency than that of natural antibodies. These “super” MIPs have higher affinities for their target and thus require lower concentrations for efficacious binding.

During synthesis, the MIPs are imprinted so as to have complementary size, shape, charge and functional groups of the selected target by using the target molecule itself (such as a polypeptide, antibody, etc.), or a substance having a very similar structure, as its “print” or “template.” MIPs can be derivatized with the same reagents afforded to antibodies. For example, fluorescent ‘super’ MIPs can be coated onto beads or wells for use in highly sensitive separations or assays, or for use in high throughput screening of proteins.

Moreover, MIPs based upon the structure of the polypeptide(s) of the present invention may be useful in screening for compounds that bind to the polypeptide(s) of the invention. Such a MIP would serve the role of a synthetic “receptor” by minimicking the native architecture of the polypeptide. In fact, the ability of a MIP to serve the role of a synthetic receptor has already been demonstrated for the estrogen receptor (Ye, L., Yu, Y., Mosbach, K, Analyst., 126(6):760-5, (2001); Dickert, F, L., Hayden, O., Halikias, K, P, Analyst., 126(6):766-71, (2001)). A synthetic receptor may either be mimicked in its entirety (e.g., as the entire protein), or mimicked as a series of short peptides corresponding to the protein (Rachkov, A., Minoura, N, Biochim, Biophys, Acta., 1544(1-2):255-66, (2001)). Such a synthetic receptor MIPs may be employed in any one or more of the screening methods described elsewhere herein.

MIPs have also been shown to be useful in “sensing” the presence of its mimicked molecule (Cheng, Z., Wang, E., Yang, X, Biosens, Bioelectron., 16(3):179-85, (2001); Jenkins, A, L., Yin, R., Jensen, J. L, Analyst., 126(6):798-802, (2001); Jenkins, A, L., Yin, R., Jensen, J. L, Analyst., 126(6):798-802, (2001)). For example, a MIP designed using a polypeptide of the present invention may be used in assays designed to identify, and potentially quantitate, the level of said polypeptide in a sample. Such a MIP may be used as a substitute for any component described in the assays, or kits, provided herein (e.g., ELISA, etc.).

A number of methods may be employed to create MIPs to a specific receptor, ligand, polypeptide, peptide, organic molecule. Several preferred methods are described by Esteban et al in J. Anal, Chem., 370(7):795-802, (2001), which is hereby incorporated herein by reference in its entirety in addition to any references cited therein. Additional methods are known in the art and are encompassed by the present invention, such as for example, Hart, B, R., Shea, K, J. J. Am. Chem, Soc., 123(9):2072-3, (2001); and Quaglia, M., Chenon, K., Hall, A, J., De, Lorenzi, E., Sellergren, B, J. Am. Chem, Soc., 123(10):2146-54, (2001); which are hereby incorporated by reference in their entirety herein.

### Uses for Antibodies Directed Against Polypeptides of the Invention

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219(1981); and Nygren, J. Histochem. And Cytochem., 30:407 (1982).

Antibodies directed against the polypeptides of the present invention are useful for the affinity purification of such polypeptides from recombinant cell culture or natural sources. In this process, the antibodies against a particular polypeptide are immobilized on a suitable support, such as a Sephadex resin or filter paper, using methods well known in the art. The immobilized antibody then is contacted with a sample containing the polypeptides to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide from the antibody.

In a preferred embodiment, antibodies directed against the polynucleotides and polypeptides of the present invention are useful for the treatment, diagnosed, and/or amelioration of immune disorders, inflammatory disorders, aberrant apoptosis, hepatic disorders, Hodgkins lymphomas, hematopoietic tumors, hyper-IgM syndromes, hypohydrotic ectodermal dysplasia, X-linked anhidrotic ectodermal dysplasia, Immunodeficiency, al incontinentia pigmenti, viral infections, HIV-1, HTLV-1, hepatitis B, hepatitis C, EBV, influenza, viral replication, host cell survival, and evasion of immune responses, rheumatoid arthritis inflammatory bowel disease, colitis, asthma, atherosclerosis, cachexia, euthyroid sick syndrome, stroke, EAE, in addition to other disorder described herein or otherwise associated with NFkB.

### Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

### Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

### Therapeutic Uses Of Antibodies

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6 M, 5×10-7 M, 10-7 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, and 10-15 M.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the gene encoding an antibody directed against a polypeptide of the present invention, said polypeptide having the potential to elicit an allergic and/or immune response in an organism, and transforming the organism with said antibody gene such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or gene therapy applications of the present invention are provided elsewhere herein.

Alternatively, antibodies of the present invention could be produced in a plant (e.g., cloning the gene of the antibody directed against a polypeptide of the present invention, and transforming a plant with a suitable vector comprising said gene for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

In another embodiment, antibodies of the present invention, preferably polyclonal antibodies, more preferably monoclonal antibodies, and most preferably single-chain antibodies, can be used as a means of inhibiting gene expression of a particular gene, or genes, in a human, mammal, and/or other organism. See, for example, International Publication Number WO 00/05391, published Feb. 3, 2000, to Dow Agrosciences LLC. The application of such methods for the antibodies of the present invention are known in the art, and are more particularly described elsewhere herein.

In yet another embodiment, antibodies of the present invention may be useful for multimerizing the polypeptides of the present invention. For example, certain proteins may confer enhanced biological activity when present in a multimeric state (i.e., such enhanced activity may be due to the increased effective concentration of such proteins whereby more protein is available in a localized location).

### Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, N.Y. (1993); and Kriegler, Gene Transfer and Expression, A Laboratory Manual, Stockton Press, N.Y. (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Nat]. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expressions of the nucleic acid is controllable by controlling the presence or absence of the appropriate inducer of transcription. Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diagnosis and Imaging With Antibodies

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell . Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

### Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

### Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because certain proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Peptide moieties may be added to the polypeptide to facilitate purification. Such regions may be removed prior to final preparation of the polypeptide. Similarly, peptide cleavage sites can be introduced in-between such peptide moieties, which could additionally be subjected to protease activity to remove said peptide(s) from the protein of the present invention. The addition of peptide moieties, including peptide cleavage sites, to facilitate handling of polypeptides are familiar and routine techniques in the art.

Moreover, polypeptides of the present invention, including fragments, and specifically epitopes, can be combined with parts of the constant domain of immunoglobulins (IgA, IgE, IgG, IgM) or portions thereof (CH1, CH2, CH3, and any combination thereof, including both entire domains and portions thereof), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of the constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the polypeptides of the present invention can be fused to marker sequences (also referred to as “tags”). Due to the availability of antibodies specific to such “tags”, purification of the fused polypeptide of the invention, and/or its identification is significantly facilitated since antibodies specific to the polypeptides of the invention are not required. Such purification may be in the form of an affinity purification whereby an anti-tag antibody or another type of affinity matrix (e.g., anti-tag antibody attached to the matrix of a flow-thru column) that binds to the epitope tag is present. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984)).

The skilled artisan would acknowledge the existence of other “tags” which could be readily substituted for the tags referred to supra for purification and/or identification of polypeptides of the present invention (Jones C., et al., J Chromatogr A. 707(1):3-22 (1995)). For example, the c-myc tag and the 8F9, 3C7, 6E10, G4m B7 and 9E10 antibodies thereto (Evan et al., Molecular and Cellular Biology 5:3610-3616 (1985)); the Herpes Simplex virus glycoprotein D (gD) tag and its antibody (Paborsky et al., Protein Engineering, 3(6):547-553 (1990), the Flag-peptide—i.e., the octapeptide sequence DYKDDDDK (SEQ ID NO: 122), (Hopp et al., Biotech. 6:1204-1210 (1988); the KT3 epitope peptide (Martin et al., Science, 255:192-194 (1992)); a-tubulin epitope peptide (Skinner et al., J. Biol. Chem., 266:15136-15166, (1991)); the T7 gene 10 protein peptide tag (Lutz-Freyermuth et al., Proc. Natl. Sci. USA, 87:6363-6397 (1990)), the FITC epitope (Zymed, Inc.), the GFP epitope (Zymed, Inc.), and the Rhodamine epitope (Zymed, Inc.).

The present invention also encompasses the attachment of up to nine codons encoding a repeating series of up to nine arginine amino acids to the coding region of a polynucleotide of the present invention. The invention also encompasses chemically derivitizing a polypeptide of the present invention with a repeating series of up to nine arginine amino acids. Such a tag, when attached to a polypeptide, has recently been shown to serve as a universal pass, allowing compounds access to the interior of cells without additional derivitization or manipulation (Wender, P., et al., unpublished data).

Protein fusions involving polypeptides of the present invention, including fragments and/or variants thereof, can be used for the following, non-limiting examples, subcellular localization of proteins, determination of protein-protein interactions via immunoprecipitation, purification of proteins via affinity chromatography, functional and/or structural characterization of protein. The present invention also encompasses the application of hapten specific antibodies for any of the uses referenced above for epitope fusion proteins. For example, the polypeptides of the present invention could be chemically derivatized to attach hapten molecules (e.g., DNP, (Zymed, Inc.)). Due to the availability of monoclonal antibodies specific to such haptens, the protein could be readily purified using immunoprecipation, for example.

Polypeptides of the present invention, including fragments and/or variants thereof, in addition to, antibodies directed against such polypeptides, fragments, and/or variants, may be fused to any of a number of known, and yet to be determined, toxins, such as ricin, saporin (Mashiba H, et al., Ann. N.Y. Acad. Sci. 1999;886:233-5), or HC toxin (Tonukari N.J., et al., Plant Cell. 2000 Feb;12(2):237-248), for example. Such fusions could be used to deliver the toxins to desired tissues for which a ligand or a protein capable of binding to the polypeptides of the invention exists.

The invention encompasses the fusion of antibodies directed against polypeptides of the present invention, including variants and fragments thereof, to said toxins for delivering the toxin to specific locations in a cell, to specific tissues, and/or to specific species. Such bifunctional antibodies are known in the art, though a review describing additional advantageous fusions, including citations for methods of production, can be found in P. J. Hudson, Curr. Opp. In. Imm. 11:548-557, (1999); this publication, in addition to the references cited therein, are hereby incorporated by reference in their entirety herein. In this context, the term “toxin” may be expanded to include any heterologous protein, a small molecule, radionucleotides, cytotoxic drugs, liposomes, adhesion molecules, glycoproteins, ligands, cell or tissue-specific ligands, enzymes, of bioactive agents, biological response modifiers, anti-fungal agents, hormones, steroids, vitamins, peptides, peptide analogs, anti-allergenic agents, anti-tubercular agents, anti-viral agents, antibiotics, anti-protozoan agents, chelates, radioactive particles, radioactive ions, X-ray contrast agents, monoclonal antibodies, polyclonal antibodies and genetic material. In view of the present disclosure, one skilled in the art could determine whether any particular “toxin” could be used in the compounds of the present invention. Examples of suitable “toxins” listed above are exemplary only and are not intended to limit the “toxins” that may be used in the present invention.

Thus, any of these above fusions can be engineered using the polynucleotides or the polypeptides of the present invention.

### Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlsbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express the polypeptide of the present invention in a eukaryotic system. Pichia pastoris is a methylotrophic yeast which can metabolize methanol as its sole carbon source. A main step in the methanol metabolization pathway is the oxidation of methanol to formaldehyde using O2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a polypeptide of the invention, as set forth herein, in a Pichea yeast system essentially as described in “Pichia Protocols: Methods in Molecular Biology” D.R. Higgins and J. Cregg, eds. The Humana Press, Totowa, N.J., 1998. This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1I/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG, as required.

In another embodiment, high-level expression of a heterologous coding sequence, such as, for example, a polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670, issued Jun. 24, 1997; U.S. Pat. No. 5,733,761, issued Mar. 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

In addition, polypeptides of the invention can be chemically synthesized using techniques known in the art (e.g., see Creighton, 1983, Proteins: Structures and Molecular Principles, W.H. Freeman & Co., N.Y., and Hunkapiller et al., Nature, 310:105-111 (1984)). For example, a polypeptide corresponding to a fragment of a polypeptide sequence of the invention can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein, the addition of epitope tagged peptide fragments (e.g., FLAG, HA, GST, thioredoxin, maltose binding protein, etc.), attachment of affinity tags such as biotin and/or streptavidin, the covalent attachment of chemical moieties to the amino acid backbone, N- or C-terminal processing of the polypeptides ends (e.g., proteolytic processing), deletion of the N-terminal methionine residue, etc.

Also provided by the invention are chemically modified derivatives of the polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The invention further encompasses chemical derivitization of the polypeptides of the present invention, preferably where the chemical is a hydrophilic polymer residue. Exemplary hydrophilic polymers, including derivatives, may be those that include polymers in which the repeating units contain one or more hydroxy groups (polyhydroxy polymers), including, for example, poly(vinyl alcohol); polymers in which the repeating units contain one or more amino groups (polyamine polymers), including, for example, peptides, polypeptides, proteins and lipoproteins, such as albumin and natural lipoproteins; polymers in which the repeating units contain one or more carboxy groups (polycarboxy polymers), including, for example, carboxymethylcellulose, alginic acid and salts thereof, such as sodium and calcium alginate, glycosaminoglycans and salts thereof, including salts of hyaluronic acid, phosphorylated and sulfonated derivatives of carbohydrates, genetic material, such as interleukin-2 and interferon, and phosphorothioate oligomers; and polymers in which the repeating units contain one or more saccharide moieties (polysaccharide polymers), including, for example, carbohydrates.

The molecular weight of the hydrophilic polymers may vary, and is generally about 50 to about 5,000,000, with polymers having a molecular weight of about 100 to about 50,000 being preferred. The polymers may be branched or unbranched. More preferred polymers have a molecular weight of about 150 to about 10,000, with molecular weights of 200 to about 8,000 being even more preferred.

For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

Additional preferred polymers which may be used to derivatize polypeptides of the invention, include, for example, poly(ethylene glycol) (PEG), poly(vinylpyrrolidine), polyoxomers, polysorbate and poly(vinyl alcohol), with PEG polymers being particularly preferred. Preferred among the PEG polymers are PEG polymers having a molecular weight of from about 100 to about 10,000. More preferably, the PEG polymers have a molecular weight of from about 200 to about 8,000, with PEG 2,000, PEG 5,000 and PEG 8,000, which have molecular weights of 2,000, 5,000 and 8,000, respectively, being even more preferred. Other suitable hydrophilic polymers, in addition to those exemplified above, will be readily apparent to one skilled in the art based on the present disclosure. Generally, the polymers used may include polymers that can be attached to the polypeptides of the invention via alkylation or acylation reactions.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved.

As with the various polymers exemplified above, it is contemplated that the polymeric residues may contain functional groups in addition, for example, to those typically involved in linking the polymeric residues to the polypeptides of the present invention. Such functionalities include, for example, carboxyl, amine, hydroxy and thiol groups. These functional groups on the polymeric residues can be further reacted, if desired, with materials that are generally reactive with such functional groups and which can assist in targeting specific tissues in the body including, for example, diseased tissue. Exemplary materials which can be reacted with the additional functional groups include, for example, proteins, including antibodies, carbohydrates, peptides, glycopeptides, glycolipids, lectins, and nucleosides.

In addition to residues of hydrophilic polymers, the chemical used to derivatize the polypeptides of the present invention can be a saccharide residue. Exemplary saccharides which can be derived include, for example, monosaccharides or sugar alcohols, such as erythrose, threose, ribose, arabinose, xylose, lyxose, fructose, sorbitol, mannitol and sedoheptulose, with preferred monosaccharides being fructose, mannose, xylose, arabinose, mannitol and sorbitol; and disaccharides, such as lactose, sucrose, maltose and cellobiose. Other saccharides include, for example, inositol and ganglioside head groups. Other suitable saccharides, in addition to those exemplified above, will be readily apparent to one skilled in the art based on the present disclosure. Generally, saccharides which may be used for derivitization include saccharides that can be attached to the polypeptides of the invention via alkylation or acylation reactions.

Moreover, the invention also encompasses derivitization of the polypeptides of the present invention, for example, with lipids (including cationic, anionic, polymerized, charged, synthetic, saturated, unsaturated, and any combination of the above, etc.). stabilizing agents.

The invention encompasses derivitization of the polypeptides of the present invention, for example, with compounds that may serve a stabilizing function (e.g., to increase the polypeptides half-life in solution, to make the polypeptides more water soluble, to increase the polypeptides hydrophilic or hydrophobic character, etc.). Polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof Accordingly, suitable polymers include, for example, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, polyethylenes (such as, for example, polyethylene glycol (including for example, the class of compounds referred to as Pluronics.RTM., commercially available from BASF, Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof. Methods for the preparation of derivatized polypeptides of the invention which employ polymers as stabilizing compounds will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

Moreover, the invention encompasses additional modifications of the polypeptides of the present invention. Such additional modifications are known in the art, and are specifically provided, in addition to methods of derivitization, etc., in U.S. Pat. No. 6,028,066, which is hereby incorporated in its entirety herein.

The polypeptides of the invention may be in monomers or multimers (i.e., dimers, trimers, tetramers and higher multimers). Accordingly, the present invention relates to monomers and multimers of the polypeptides of the invention, their preparation, and compositions (preferably, Therapeutics) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only polypeptides corresponding to the amino acid sequence of 109-118, 126, 128, 144-152, or 160-161 (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the sequence listing). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, osteoprotegerin (see, e.g., International Publication NO: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occurring trimeric proteins may be employed in preparing trimeric polypeptides of the invention.

In another example, proteins of the invention are associated by interactions between Flag® polypeptide sequence contained in fusion proteins of the invention containing Flag® polypeptide sequence. In a further embodiment, associations proteins of the invention are associated by interactions between heterologous polypeptide sequence contained in Flag® fusion proteins of the invention and anti-Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hydrophobic or signal peptide) and which can be incorporated by membrane reconstitution techniques into liposomes (see, e.g., U.S. Pat. No. 5,478,925, which is herein incorporated by reference in its entirety).

In addition, the polynucleotide insert of the present invention could be operatively linked to “artificial” or chimeric promoters and transcription factors. Specifically, the artificial promoter could comprise, or alternatively consist, of any combination of cis-acting DNA sequence elements that are recognized by trans-acting transcription factors. Preferably, the cis acting DNA sequence elements and trans-acting transcription factors are operable in mammals. Further, the trans-acting transcription factors of such “artificial” promoters could also be “artificial” or chimeric in design themselves and could act as activators or repressors to said “artificial” promoter.

### Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in SEQ ID NO:1-108, 125, 127, 132-140, 158-159, or 264-284. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:1-108, 125, 127, 132-140, 158-159, or 264-284 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. Disease mapping data are known in the art. Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected organisms can be examined. First, visible structural alterations in the chromosomes, such as deletions or translocations, are examined in chromosome spreads or by PCR. If no structural alterations exist, the presence of point mutations are ascertained. Mutations observed in some or all affected organisms, but not in normal organisms, indicates that the mutation may cause the disease. However, complete sequencing of the polypeptide and the corresponding gene from several normal organisms is required to distinguish the mutation from a polymorphism. If a new polymorphism is identified, this polymorphic polypeptide can be used for further linkage analysis.

Furthermore, increased or decreased expression of the gene in affected organisms as compared to unaffected organisms can be assessed using polynucleotides of the present invention. Any of these alterations (altered expression, chromosomal rearrangement, or mutation) can be used as a diagnostic or prognostic marker.

Thus, the invention also provides a diagnostic method useful during diagnosis of a disorder, involving measuring the expression level of polynucleotides of the present invention in cells or body fluid from an organism and comparing the measured gene expression level with a standard level of polynucleotide expression level, whereby an increase or decrease in the gene expression level compared to the standard is indicative of a disorder.

By “measuring the expression level of a polynucleotide of the present invention” is intended qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). Preferably, the polypeptide level or mRNA level in the first biological sample is measured or estimated and compared to a standard polypeptide level or mRNA level, the standard being taken from a second biological sample obtained from an individual not having the disorder or being determined by averaging levels from a population of organisms not having a disorder. As will be appreciated in the art, once a standard polypeptide level or mRNA level is known, it can be used repeatedly as a standard for comparison.

By “biological sample” is intended any biological sample obtained from an organism, body fluids, cell line, tissue culture, or other source which contains the polypeptide of the present invention or mRNA. As indicated, biological samples include body fluids (such as the following non-limiting examples, sputum, amniotic fluid, urine, saliva, breast milk, secretions, interstitial fluid, blood, serum, spinal fluid, etc.) which contain the polypeptide of the present invention, and other tissue sources found to express the polypeptide of the present invention. Methods for obtaining tissue biopsies and body fluids from organisms are well known in the art. Where the biological sample is to include mRNA, a tissue biopsy is the preferred source.

The method(s) provided above may Preferably be applied in a diagnostic method and/or kits in which polynucleotides and/or polypeptides are attached to a solid support. In one exemplary method, the support may be a “gene chip” or a “biological chip” as described in U.S. Pat. Nos. 5,837,832, 5,874,219, and 5,856,174. Further, such a gene chip with polynucleotides of the present invention attached may be used to identify polymorphisms between the polynucleotide sequences, with polynucleotides isolated from a test subject. The knowledge of such polymorphisms (i.e. their location, as well as, their existence) would be beneficial in identifying disease loci for many disorders, including proliferative diseases and conditions. Such a method is described in U.S. Pat. Nos. 5,858,659 and 5,856,104. The U.S. patent referenced supra are hereby incorporated by reference in their entirety herein.

The present invention encompasses polynucleotides of the present invention that are chemically synthesized, or reproduced as peptide nucleic acids (PNA), or according to other methods known in the art. The use of PNAs would serve as the preferred form if the polynucleotides are incorporated onto a solid support, or gene chip. For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by P. E. Nielsen, M. Egholm, R. H. Berg and O. Buchardt, Science 254, 1497 (1991); and M. Egholm, O. Buchardt, L. Christensen, C. Behrens, S. M. Freier, D. A. Driver, R. H. Berg, S. K. Kim, B. Norden, and P. E. Nielsen, Nature 365, 666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the stronger binding characteristics of PNA:DNA hybrids. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

In addition to the foregoing, a polynucleotide can be used to control gene expression through triple helix formation or antisense DNA or RNA. Antisense techniques are discussed, for example, in Okano, J. Neurochem. 56: 560 (1991); “Oligodeoxynucleotides as Antisense Inhibitors of Gene Expression, CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). Both methods rely on binding of the polynucleotide to a complementary DNA or RNA. For these techniques, preferred polynucleotides are usually oligonucleotides 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat or prevent disease.

The present invention encompasses the addition of a nuclear localization signal, operably linked to the 5′ end, 3′ end, or any location therein, to any of the oligonucleotides, antisense oligonucleotides, triple helix oligonucleotides, ribozymes, PNA oligonucleotides, and/or polynucleotides, of the present invention. See, for example, G. Cutrona, et al., Nat. Biotech., 18:300-303, (2000); which is hereby incorporated herein by reference.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell. In one example, polynucleotide sequences of the present invention may be used to construct chimeric RNA/DNA oligonucleotides corresponding to said sequences, specifically designed to induce host cell mismatch repair mechanisms in an organism upon systemic injection, for example (Bartlett, R. J., et al., Nat. Biotech, 18:615-622 (2000), which is hereby incorporated by reference herein in its entirety). Such RNA/DNA oligonucleotides could be designed to correct genetic defects in certain host strains, and/or to introduce desired phenotypes in the host (e.g., introduction of a specific polymorphism within an endogenous gene corresponding to a polynucleotide of the present invention that may ameliorate and/or prevent a disease symptom and/or disorder, etc.). Alternatively, the polynucleotide sequence of the present invention may be used to construct duplex oligonucleotides corresponding to said sequence, specifically designed to correct genetic defects in certain host strains, and/or to introduce desired phenotypes into the host (e.g., introduction of a specific polymorphism within an endogenous gene corresponding to a polynucleotide of the present invention that may ameliorate and/or prevent a disease symptom and/or disorder, etc). Such methods of using duplex oligonucleotides are known in the art and are encompassed by the present invention (see EP1007712, which is hereby incorporated by reference herein in its entirety).

The polynucleotides are also useful for identifying organisms from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an organisms genome. These sequences can be used to prepare PCR primers for amplifying and isolating such selected DNA, which can then be sequenced. Using this technique, organisms can be identified because each organism will have a unique set of DNA sequences. Once an unique ID database is established for an organism, positive identification of that organism, living or dead, can be made from extremely small tissue samples. Similarly, polynucleotides of the present invention can be used as polymorphic markers, in addition to, the identification of transformed or non-transformed cells and/or tissues.

There is also a need for reagents capable of identifying the source of a particular tissue. Such need arises, for example, when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination. Moreover, as mentioned above, such reagents can be used to screen and/or identify transformed and non-transformed cells and/or tissues.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

### Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell . Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detecTable I and IIImaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Moreover, polypeptides of the present invention can be used to treat, prevent, and/or diagnose disease. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B, SOD, catalase, DNA repair proteins), to inhibit the activity of a polypeptide (e.g., an oncogene or tumor suppressor), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth inhibition, enhancement of the immune response to proliferative cells or tissues).

Similarly, antibodies directed to a polypeptide of the present invention can also be used to treat, prevent, and/or diagnose disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

### Gene Therapy Methods

Another aspect of the present invention is to gene therapy methods for treating or preventing disorders, diseases and conditions. The gene therapy methods relate to the introduction of nucleic acid (DNA, RNA and antisense DNA or RNA) sequences into an animal to achieve expression of a polypeptide of the present invention. This method requires a polynucleotide which codes for a polypeptide of the invention that operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a polynucleotide of the invention ex vivo, with the engineered cells then being provided to a patient to be treated with the polypeptide. Such methods are well-known in the art. For example, see Belldegrun et al., J. Natl. Cancer Inst., 85:207-216 (1993); Ferrantini et al., Cancer Research, 53:107-1112 (1993); Ferrantini et al., J. Immunology 153: 4604-4615 (1994); Kaido, T., et al., Int. J. Cancer 60: 221-229 (1995); Ogura et al., Cancer Research 50: 5102-5106 (1990); Santodonato, et al., Human Gene Therapy 7:1-10 (1996); Santodonato, et al., Gene Therapy 4:1246-1255 (1997); and Zhang, et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the polynucleotide constructs can be delivered by any method that delivers injectable materials to the cells of an animal, such as, injection into the interstitial space of tissues (heart, muscle, skin, lung, liver, and the like). The polynucleotide constructs may be delivered in a pharmaceutically acceptable liquid or aqueous carrier.

In one embodiment, the polynucleotide of the invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the polynucleotides of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The polynucleotide vector constructs of the invention used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of polynucleotide sequence of the invention. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for the polynucleotides of the invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct of the invention can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs may also be delivered with delivery vehicles such as viral sequences, viral particles, liposome formulations, lipofectin, precipitating agents, etc. Such methods of delivery are known in the art.

In certain embodiments, the polynucleotide constructs of the invention are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Feigner et al., Proc. Natl. Acad. Sci. USA, 84:7413-7416 (1987), which is herein incorporated by reference); mRNA (Malone et al., Proc. Natl. Acad. Sci. USA, 86:6077-6081 (1989), which is herein incorporated by reference); and purified transcription factors (Debs et al., J. Biol. Chem., 265:10189-10192 (1990), which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Feigner et al., Proc. Natl. Acad. Sci. USA, 84:7413-7416 (1987), which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication NO: WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., Felgner et al., Proc. Natl. Acad. Sci. USA, 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), diolcoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology, 101:512-527 (1983), which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca2+-EDTA chelation (Papahadjopoulos et al., Biochim. Biophys. Acta, 394:483 (1975); Wilson et al., Cell, 17:77 (1979)); ether injection (Deamer et al., Biochim. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch et al., Proc. Natl. Acad. Sci. USA, 76:145 (1979)); and reverse-phase evaporation (REV) (Fraley et al., J. Biol. Chem., 255:10431 (1980); Szoka et al., Proc. Natl. Acad. Sci. USA, 75:145 (1978); Schaefer-Ridder et al., Science, 215:166 (1982)), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals.

In certain embodiments, cells are engineered, ex vivo or in vivo, using a retroviral particle containing RNA which comprises a sequence encoding polypeptides of the invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy, 1:5-14 (1990), which is incorporated herein by reference in its entirety. The vector may transduce the packaging cells through any means known in the art. Such means include, but are not limited to, electroporation, the use of liposomes, and CaPO4 precipitation. In one alternative, the retroviral plasmid vector may be encapsulated into a liposome, or coupled to a lipid, and then administered to a host.

The producer cell line generates infectious retroviral vector particles which include polynucleotide encoding polypeptides of the invention. Such retroviral vector particles then may be employed, to transduce eukaryotic cells, either in vitro or in vivo. The transduced eukaryotic cells will express polypeptides of the invention.

In certain other embodiments, cells are engineered, ex vivo or in vivo, with polynucleotides of the invention contained in an adenovirus vector. Adenovirus can be manipulated such that it encodes and expresses polypeptides of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartzet al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in human cancer were uniformly negative (Green et al. Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, Curr. Topics in Microbiol. Immunol., 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct containing polynucleotides of the invention is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct of the invention. These viral particles are then used to transduce eukaryotic cells, either ex vivo or in vivo. The transduced cells will contain the polynucleotide construct integrated into its genome, and will express the desired gene product.

Another method of gene therapy involves operably associating heterologous control regions and endogenous polynucleotide sequences (e.g. encoding the polypeptide sequence of interest) via homologous recombination (see, e.g., U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotides encoding polypeptides of the present invention may be administered along with other polynucleotides encoding angiogenic proteins. Angiogenic proteins include, but are not limited to, acidic and basic fibroblast growth factors, VEGF-1, VEGF-2 (VEGF-C), VEGF-3 (VEGF-B), epidermal growth factor alpha and beta, platelet-derived endothelial cell growth factor, platelet-derived growth factor, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding a polypeptide of the invention contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers. (Kaneda et al., Science, 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA, 189:11277-11281 (1992), which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention can be administered to any animal, preferably to mammals and birds. Preferred mammals include humans, dogs, cats, mice, rats, rabbits sheep, cattle, horses and pigs, with humans being particularly preferred.

### Biological Activities

The polynucleotides or polypeptides, or agonists or antagonists of the present invention can be used in assays to test for one or more biological activities. If these polynucleotides and polypeptides do exhibit activity in a particular assay, it is likely that these molecules may be involved in the diseases associated with the biological activity. Thus, the polynucleotides or polypeptides, or agonists or antagonists could be used to treat the associated disease.

### Immune Activity

The polynucleotides or polypeptides, or agonists or antagonists of the present invention may be useful in treating, preventing, and/or diagnosing diseases, disorders, and/or conditions of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune diseases, disorders, and/or conditions may be genetic, somatic, such as cancer or some autoimmune diseases, disorders, and/or conditions, acquired (e.g., by chemotherapy or toxins), or infectious. Moreover, a polynucleotides or polypeptides, or agonists or antagonists of the present invention can be used as a marker or detector of a particular immune system disease or disorder.

A polynucleotides or polypeptides, or agonists or antagonists of the present invention may be useful in treating, preventing, and/or diagnosing diseases, disorders, and/or conditions of hematopoietic cells. A polynucleotides or polypeptides, or agonists or antagonists of the present invention could be used to increase differentiation and proliferation of hematopoietic cells, including the pluripotent stem cells, in an effort to treat or prevent those diseases, disorders, and/or conditions associated with a decrease in certain (or many) types hematopoietic cells. Examples of immunologic deficiency syndromes include, but are not limited to: blood protein diseases, disorders, and/or conditions (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a polynucleotides or polypeptides, or agonists or antagonists of the present invention could also be used to modulate hemostatic (the stopping of bleeding) or thrombolytic activity (clot formation). For example, by increasing hemostatic or thrombolytic activity, a polynucleotides or polypeptides, or agonists or antagonists of the present invention could be used to treat or prevent blood coagulation diseases, disorders, and/or conditions (e.g., afibrinogenemia, factor deficiencies, arterial thrombosis, venous thrombosis, etc.), blood platelet diseases, disorders, and/or conditions (e.g. thrombocytopenia), or wounds resulting from trauma, surgery, or other causes. Alternatively, a polynucleotides or polypeptides, or agonists or antagonists of the present invention that can decrease hemostatic or thrombolytic activity could be used to inhibit or dissolve clotting. Polynucleotides or polypeptides, or agonists or antagonists of the present invention are may also be useful for the detection, prognosis, treatment, and/or prevention of heart attacks (infarction), strokes, scarring, fibrinolysis, uncontrolled bleeding, uncontrolled coagulation, uncontrolled complement fixation, and/or inflammation.

A polynucleotides or polypeptides, or agonists or antagonists of the present invention may also be useful in treating, preventing, and/or diagnosing autoimmune diseases, disorders, and/or conditions. Many autoimmune diseases, disorders, and/or conditions result from inappropriate recognition of self as foreign material by immune cells. This inappropriate recognition results in an immune response leading to the destruction of the host tissue. Therefore, the administration of a polynucleotides or polypeptides, or agonists or antagonists of the present invention that inhibits an immune response, particularly the proliferation, differentiation, or chemotaxis of T-cells, may be an effective therapy in preventing autoimmune diseases, disorders, and/or conditions.

Examples of autoimmune diseases, disorders, and/or conditions that can be treated, prevented, and/or diagnosed or detected by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated, prevented, and/or diagnosed by polynucleotides or polypeptides, or agonists or antagonists of the present invention. Moreover, these molecules can be used to treat anaphylaxis, hypersensitivity to an antigenic molecule, or blood group incompatibility.

A polynucleotides or polypeptides, or agonists or antagonists of the present invention may also be used to treat, prevent, and/or diagnose organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a polynucleotides or polypeptides, or agonists or antagonists of the present invention that inhibits an immune response, particularly the proliferation, differentiation, or chemotaxis of T-cells, may be an effective therapy in preventing organ rejection or GVHD.

Similarly, a polynucleotides or polypeptides, or agonists or antagonists of the present invention may also be used to modulate inflammation. For example, the polypeptide or polynucleotide or agonists or antagonist may inhibit the proliferation and differentiation of cells involved in an inflammatory response. These molecules can be used to treat, prevent, and/or diagnose inflammatory conditions, both chronic and acute conditions, including chronic prostatitis, granulomatous prostatitis and malacoplakia, inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

### Hyperproliferative Disorders

A polynucleotides or polypeptides, or agonists or antagonists of the invention can be used to treat, prevent, and/or diagnose hyperproliferative diseases, disorders, and/or conditions, including neoplasms. A polynucleotides or polypeptides, or agonists or antagonists of the present invention may inhibit the proliferation of the disorder through direct or indirect interactions. Alternatively, a polynucleotides or polypeptides, or agonists or antagonists of the present invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative diseases, disorders, and/or conditions can be treated, prevented, and/or diagnosed. This immune response may be increased by either enhancing an existing immune response, or by initiating a new immune response. Alternatively, decreasing an immune response may also be a method of treating, preventing, and/or diagnosing hyperproliferative diseases, disorders, and/or conditions, such as a chemotherapeutic agent.

Examples of hyperproliferative diseases, disorders, and/or conditions that can be treated, prevented, and/or diagnosed by polynucleotides or polypeptides, or agonists or antagonists of the present invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative diseases, disorders, and/or conditions can also be treated, prevented, and/or diagnosed by a polynucleotides or polypeptides, or agonists or antagonists of the present invention. Examples of such hyperproliferative diseases, disorders, and/or conditions include, but are not limited to: hypergammaglobulinemia, lymphoproliferative diseases, disorders, and/or conditions, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

One preferred embodiment utilizes polynucleotides of the present invention to inhibit aberrant cellular division, by gene therapy using the present invention, and/or protein fusions or fragments thereof.

Thus, the present invention provides a method for treating or preventing cell proliferative diseases, disorders, and/or conditions by inserting into an abnormally proliferating cell a polynucleotide of the present invention, wherein said polynucleotide represses said expression.

Another embodiment of the present invention provides a method of treating or preventing cell-proliferative diseases, disorders, and/or conditions in individuals comprising administration of one or more active gene copies of the present invention to an abnormally proliferating cell or cells. In a preferred embodiment, polynucleotides of the present invention is a DNA construct comprising a recombinant expression vector effective in expressing a DNA sequence encoding said polynucleotides. In another preferred embodiment of the present invention, the DNA construct encoding the polynucleotides of the present invention is inserted into cells to be treated utilizing a retrovirus, or more Preferably an adenoviral vector (See G J. Nabel, et. al., PNAS 1999 96: 324-326, which is hereby incorporated by reference). In a most preferred embodiment, the viral vector is defective and will not transform non-proliferating cells, only proliferating cells. Moreover, in a preferred embodiment, the polynucleotides of the present invention inserted into proliferating cells either alone, or in combination with or fused to other polynucleotides, can then be modulated via an external stimulus (i.e. magnetic, specific small molecule, chemical, or drug administration, etc.), which acts upon the promoter upstream of said polynucleotides to induce expression of the encoded protein product. As such the beneficial therapeutic affect of the present invention may be expressly modulated (i.e. to increase, decrease, or inhibit expression of the present invention) based upon said external stimulus.

Polynucleotides of the present invention may be useful in repressing expression of oncogenic genes or antigens. By “repressing expression of the oncogenic genes” is intended the suppression of the transcription of the gene, the degradation of the gene transcript (pre-message RNA), the inhibition of splicing, the destruction of the messenger RNA, the prevention of the post-translational modifications of the protein, the destruction of the protein, or the inhibition of the normal function of the protein.

For local administration to abnormally proliferating cells, polynucleotides of the present invention may be administered by any method known to those of skill in the art including, but not limited to transfection, electroporation, microinjection of cells, or in vehicles such as liposomes, lipofectin, or as naked polynucleotides, or any other method described throughout the specification. The polynucleotide of the present invention may be delivered by known gene delivery systems such as, but not limited to, retroviral vectors (Gilboa, J. Virology 44:845 (1982); Hocke, Nature 320:275 (1986); Wilson, et al., Proc. Natl. Acad. Sci. U.S.A. 85:3014), vaccinia virus system (Chakrabarty et al., Mol. Cell Biol. 5:3403 (1985) or other efficient DNA delivery systems (Yates et al., Nature 313:812 (1985)) known to those skilled in the art. These references are exemplary only and are hereby incorporated by reference. In order to specifically deliver or transfect cells which are abnormally proliferating and spare non-dividing cells, it is preferable to utilize a retrovirus, or adenoviral (as described in the art and elsewhere herein) delivery system known to those of skill in the art. Since host DNA replication is required for retroviral DNA to integrate and the retrovirus will be unable to self replicate due to the lack of the retrovirus genes needed for its life cycle. Utilizing such a retroviral delivery system for polynucleotides of the present invention will target said gene and constructs to abnormally proliferating cells and will spare the non-dividing normal cells.

The polynucleotides of the present invention may be delivered directly to cell proliferative disorder/disease sites in internal organs, body cavities and the like by use of imaging devices used to guide an injecting needle directly to the disease site. The polynucleotides of the present invention may also be administered to disease sites at the time of surgical intervention.

By “cell proliferative disease” is meant any human or animal disease or disorder, affecting any one or any combination of organs, cavities, or body parts, which is characterized by single or multiple local abnormal proliferations of cells, groups of cells, or tissues, whether benign or malignant.

Any amount of the polynucleotides of the present invention may be administered as long as it has a biologically inhibiting effect on the proliferation of the treated cells. Moreover, it is possible to administer more than one of the polynucleotide of the present invention simultaneously to the same site. By “biologically inhibiting” is meant partial or total growth inhibition as well as decreases in the rate of proliferation or growth of the cells. The biologically inhibitory dose may be determined by assessing the effects of the polynucleotides of the present invention on target malignant or abnormally proliferating cell growth in tissue culture, tumor growth in animals and cell cultures, or any other method known to one of ordinary skill in the art.

The present invention is further directed to antibody-based therapies which involve administering of anti-polypeptides and anti-polynucleotide antibodies to a mammalian, preferably human, patient for treating, preventing, and/or diagnosing one or more of the described diseases, disorders, and/or conditions. Methods for producing anti-polypeptides and anti-polynucleotide antibodies polyclonal and monoclonal antibodies are described in detail elsewhere herein. Such antibodies may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

In particular, the antibodies, fragments and derivatives of the present invention are useful for treating, preventing, and/or diagnosing a subject having or developing cell proliferative and/or differentiation diseases, disorders, and/or conditions as described herein. Such treatment comprises administering a single or multiple doses of the antibody, or a fragment, derivative, or a conjugate thereof.

The antibodies of this invention may be advantageously utilized in combination with other monoclonal or chimeric antibodies, or with lymphokines or hematopoietic growth factors, for example, which serve to increase the number or activity of effector cells which interact with the antibodies.

It is preferred to use high affinity and/or potent in vivo inhibiting and/or neutralizing antibodies against polypeptides or polynucleotides of the present invention, fragments or regions thereof, for both immunoassays directed to and therapy of diseases, disorders, and/or conditions related to polynucleotides or polypeptides, including fragments thereof, of the present invention. Such antibodies, fragments, or regions, will preferably have an affinity for polynucleotides or polypeptides, including fragments thereof. Preferred binding affinities include those with a dissociation constant or Kd less than 5×10-6M, 10-6M, 5×10-7M, 10-7M, 5×10-8M, 10-8M, 5×10-9M, 10-9M, 5×10-10M, 10-10M, 5×10-11M, 10-11M, 5×10-12M, 10-12M, 5×10-13M, 10-13M, 5×10-14M, 10-14M, 5×10-15M, and 10-15M.

Moreover, polypeptides of the present invention may be useful in inhibiting the angiogenesis of proliferative cells or tissues, either alone, as a protein fusion, or in combination with other polypeptides directly or indirectly, as described elsewhere herein. In a most preferred embodiment, said anti-angiogenesis effect may be achieved indirectly, for example, through the inhibition of hematopoietic, tumor-specific cells, such as tumor-associated macrophages (See Joseph I B, et al. J Natl Cancer Inst, 90(21):1648-53 (1998), which is hereby incorporated by reference). Antibodies directed to polypeptides or polynucleotides of the present invention may also result in inhibition of angiogenesis directly, or indirectly (See Witte L, et al., Cancer Metastasis Rev. 17(2):155-61 (1998), which is hereby incorporated by reference)).

Polypeptides, including protein fusions, of the present invention, or fragments thereof may be useful in inhibiting proliferative cells or tissues through the induction of apoptosis. Said polypeptides may act either directly, or indirectly to induce apoptosis of proliferative cells and tissues, for example in the activation of a death-domain receptor, such as tumor necrosis factor (TNF) receptor-1, CD95 (Fas/APO-1), TNF-receptor-related apoptosis-mediated protein (TRAMP) and TNF-related apoptosis-inducing ligand (TRAIL) receptor-1 and -2 (See Schulze-Osthoff K, et al., Eur J Biochem 254(3):439-59 (1998), which is hereby incorporated by reference). Moreover, in another preferred embodiment of the present invention, said polypeptides may induce apoptosis through other mechanisms, such as in the activation of other proteins which will activate apoptosis, or through stimulating the expression of said proteins, either alone or in combination with small molecule drugs or adjuvants, such as apoptonin, galectins, thioredoxins, antiinflammatory proteins (See for example, Mutat. Res. 400(1-2):447-55 (1998), Med Hypotheses.50(5):423-33 (1998), Chem. Biol. Interact. April 24;1 11-112:23-34 (1998), J Mol Med.76(6):402-12 (1998), Int. J. Tissue React. 20(l):3-15 (1998), which are all hereby incorporated by reference).

Polypeptides, including protein fusions to, or fragments thereof, of the present invention are useful in inhibiting the metastasis of proliferative cells or tissues. Inhibition may occur as a direct result of administering polypeptides, or antibodies directed to said polypeptides as described elsewhere herein, or indirectly, such as activating the expression of proteins known to inhibit metastasis, for example alpha 4 integrins, (See, e.g., Curr Top Microbiol Immunol 1998;231:125-41, which is hereby incorporated by reference). Such therapeutic affects of the present invention may be achieved either alone, or in combination with small molecule drugs or adjuvants.

In another embodiment, the invention provides a method of delivering compositions containing the polypeptides of the invention (e.g., compositions containing polypeptides or polypeptide antibodies associated with heterologous polypeptides, heterologous nucleic acids, toxins, or prodrugs) to targeted cells expressing the polypeptide of the present invention. Polypeptides or polypeptide antibodies of the invention may be associated with heterologous polypeptides, heterologous nucleic acids, toxins, or prodrugs via hydrophobic, hydrophilic, ionic and/or covalent interactions.

Polypeptides, protein fusions to, or fragments thereof, of the present invention are useful in enhancing the immunogenicity and/or antigenicity of proliferating cells or tissues, either directly, such as would occur if the polypeptides of the present invention ‘vaccinated’ the immune response to respond to proliferative antigens and immunogens, or indirectly, such as in activating the expression of proteins known to enhance the immune response (e.g. chemokines), to said antigens and immunogens.

### Cardiovascular Disorders

Polynucleotides or polypeptides, or agonists or antagonists of the invention may be used to treat, prevent, and/or diagnose cardiovascular diseases, disorders, and/or conditions, including peripheral artery disease, such as limb ischemia.

Cardiovascular diseases, disorders, and/or conditions also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular diseases, disorders, and/or conditions include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboanguitis obliterans, hypersensitivity vasculitis, Schoenlein-Henoch purpura, allergic cutaneous vasculitis, and Wegener's granulomatosis.

Polynucleotides or polypeptides, or agonists or antagonists of the invention, are especially effective for the treatment of critical limb ischemia and coronary disease.

Polypeptides may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Polypeptides of the invention may be administered as part of a Therapeutic, described in more detail below. Methods of delivering polynucleotides of the invention are described in more detail herein.

### Diseases at the Cellular Level

Diseases associated with increased cell survival or the inhibition of apoptosis that could be treated, prevented, and/or diagnosed by the polynucleotides or polypeptides and/or antagonists or agonists of the invention, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune diseases, disorders, and/or conditions (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection. In preferred embodiments, the polynucleotides or polypeptides, and/or agonists or antagonists of the invention are used to inhibit growth, progression, and/or metastasis of cancers, in particular those listed above.

Additional diseases or conditions associated with increased cell survival that could be treated, prevented or diagnosed by the polynucleotides or polypeptides, or agonists or antagonists of the invention, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated, prevented, and/or diagnosed by the polynucleotides or polypeptides, and/or agonists or antagonists of the invention, include AIDS; neurodegenerative diseases, disorders, and/or conditions (such as Alzheimer's disease, Parkinson's disease, Amyotrophic lateral sclerosis, Retinitis pigmentosa, Cerebellar degeneration and brain tumor or prior associated disease); autoimmune diseases, disorders, and/or conditions (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

### Wound Healing and Epithelial Cell Proliferation

In accordance with yet a further aspect of the present invention, there is provided a process for utilizing the polynucleotides or polypeptides, and/or agonists or antagonists of the invention, for therapeutic purposes, for example, to stimulate epithelial cell proliferation and basal keratinocytes for the purpose of wound healing, and to stimulate hair follicle production and healing of dermal wounds. Polynucleotides or polypeptides, as well as agonists or antagonists of the invention, may be clinically useful in stimulating wound healing including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from heat exposure or chemicals, and other abnormal wound healing conditions such as uremia, malnutrition, vitamin deficiencies and complications associated with systemic treatment with steroids, radiation therapy and antineoplastic drugs and antimetabolites. Polynucleotides or polypeptides, and/or agonists or antagonists of the invention, could be used to promote dermal reestablishment subsequent to dermal loss

The polynucleotides or polypeptides, and/or agonists or antagonists of the invention, could be used to increase the adherence of skin grafts to a wound bed and to stimulate re-epithelialization from the wound bed. The following are a non-exhaustive list of grafts that polynucleotides or polypeptides, agonists or antagonists of the invention, could be used to increase adherence to a wound bed: autografts, artificial skin, allografts, autodermic graft, autoepidermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft. The polynucleotides or polypeptides, and/or agonists or antagonists of the invention, can be used to promote skin strength and to improve the appearance of aged skin.

It is believed that the polynucleotides or polypeptides, and/or agonists or antagonists of the invention, will also produce changes in hepatocyte proliferation, and epithelial cell proliferation in the lung, breast, pancreas, stomach, small intestine, and large intestine. The polynucleotides or polypeptides, and/or agonists or antagonists of the invention, could promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. The polynucleotides or polypeptides, and/or agonists or antagonists of the invention, may promote proliferation of endothelial cells, keratinocytes, and basal keratinocytes.

The polynucleotides or polypeptides, and/or agonists or antagonists of the invention, could also be used to reduce the side effects of gut toxicity that result from radiation, chemotherapy treatments or viral infections. The polynucleotides or polypeptides, and/or agonists or antagonists of the invention, may have a cytoprotective effect on the small intestine mucosa. The polynucleotides or polypeptides, and/or agonists or antagonists of the invention, may also stimulate healing of mucositis (mouth ulcers) that result from chemotherapy and viral infections.

The polynucleotides or polypeptides, and/or agonists or antagonists of the invention, could further be used in full regeneration of skin in full and partial thickness skin defects, including burns, (i.e., repopulation of hair follicles, sweat glands, and sebaceous glands), treatment of other skin defects such as psoriasis. The polynucleotides or polypeptides, and/or agonists or antagonists of the invention, could be used to treat epidermolysis bullosa, a defect in adherence of the epidermis to the underlying dermis which results in frequent, open and painful blisters by accelerating reepithelialization of these lesions. The polynucleotides or polypeptides, and/or agonists or antagonists of the invention, could also be used to treat gastric and doudenal ulcers and help heal by scar formation of the mucosal lining and regeneration of glandular mucosa and duodenal mucosal lining more rapidly. Inflamamatory bowel diseases, such as Crohn's disease and ulcerative colitis, are diseases which result in destruction of the mucosal surface of the small or large intestine, respectively. Thus, the polynucleotides or polypeptides, and/or agonists or antagonists of the invention, could be used to promote the resurfacing of the mucosal surface to aid more rapid healing and to prevent progression of inflammatory bowel disease. Treatment with the polynucleotides or polypeptides, and/or agonists or antagonists of the invention, is expected to have a significant effect on the production of mucus throughout the gastrointestinal tract and could be used to protect the intestinal mucosa from injurious substances that are ingested or following surgery. The polynucleotides or polypeptides, and/or agonists or antagonists of the invention, could be used to treat diseases associate with the under expression of the polynucleotides of the invention.

Moreover, the polynucleotides or polypeptides, and/or agonists or antagonists of the invention, could be used to prevent and heal damage to the lungs due to various pathological states. A growth factor such as the polynucleotides or polypeptides, and/or agonists or antagonists of the invention, which could stimulate proliferation and differentiation and promote the repair of alveoli and brochiolar epithelium to prevent or treat acute or chronic lung damage. For example, emphysema, which results in the progressive loss of aveoli, and inhalation injuries, i.e., resulting from smoke inhalation and burns, that cause necrosis of the bronchiolar epithelium and alveoli could be effectively treated, prevented, and/or diagnosed using the polynucleotides or polypeptides, and/or agonists or antagonists of the invention. Also, the polynucleotides or polypeptides, and/or agonists or antagonists of the invention, could be used to stimulate the proliferation of and differentiation of type II pneumocytes, which may help treat or prevent disease such as hyaline membrane diseases, such as infant respiratory distress syndrome and bronchopulmonary displasia, in premature infants.

The polynucleotides or polypeptides, and/or agonists or antagonists of the invention, could stimulate the proliferation and differentiation of hepatocytes and, thus, could be used to alleviate or treat liver diseases and pathologies such as fulminant liver failure caused by cirrhosis, liver damage caused by viral hepatitis and toxic substances (i.e., acetaminophen, carbon tetraholoride and other hepatotoxins known in the art).

In addition, the polynucleotides or polypeptides, and/or agonists or antagonists of the invention, could be used treat or prevent the onset of diabetes mellitus. In patients with newly diagnosed Types I and II diabetes, where some islet cell function remains, the polynucleotides or polypeptides, and/or agonists or antagonists of the invention, could be used to maintain the islet function so as to alleviate, delay or prevent permanent manifestation of the disease. Also, the polynucleotides or polypeptides, and/or agonists or antagonists of the invention, could be used as an auxiliary in islet cell transplantation to improve or promote islet cell function.

### Neurological Diseases

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

### Infectious Disease

A polypeptide or polynucleotide and/or agonist or antagonist of the present invention can be used to treat, prevent, and/or diagnose infectious agents. For example, by increasing the immune response, particularly increasing the proliferation and differentiation of B and/or T cells, infectious diseases may be treated, prevented, and/or diagnosed. The immune response may be increased by either enhancing an existing immune response, or by initiating a new immune response. Alternatively, polypeptide or polynucleotide and/or agonist or antagonist of the present invention may also directly inhibit the infectious agent, without necessarily eliciting an immune response.

Viruses are one example of an infectious agent that can cause disease or symptoms that can be treated, prevented, and/or diagnosed by a polynucleotide or polypeptide and/or agonist or antagonist of the present invention. Examples of viruses, include, but are not limited to Examples of viruses, include, but are not limited to the following DNA and RNA viruses and viral families: Arbovirus, Adenoviridae, Arenaviridae, Arterivirus, Birnaviridae, Bunyaviridae, Caliciviridae, Circoviridae, Coronaviridae, Dengue, EBV, HIV, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza A, Influenza B, and parainfluenza), Papiloma virus, Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, respiratory syncytial virus, encephalitis, eye infections (e.g., conjunctivitis, keratitis), chronic fatigue syndrome, hepatitis (A, B, C, E, Chronic Active, Delta), Japanese B encephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. polynucleotides or polypeptides, or agonists or antagonists of the invention, can be used to treat, prevent, and/or diagnose any of these symptoms or diseases. In specific embodiments, polynucleotides, polypeptides, or agonists or antagonists of the invention are used to treat, prevent, and/or diagnose: meningitis, Dengue, EBV, and/or hepatitis (e.g., hepatitis B). In an additional specific embodiment polynucleotides, polypeptides, or agonists or antagonists of the invention are used to treat patients nonresponsive to one or more other commercially available hepatitis vaccines. In a further specific embodiment polynucleotides, polypeptides, or agonists or antagonists of the invention are used to treat, prevent, and/or diagnose AIDS.

Similarly, bacterial or fungal agents that can cause disease or symptoms and that can be treated, prevented, and/or diagnosed by a polynucleotide or polypeptide and/or agonist or antagonist of the present invention include, but not limited to, include, but not limited to, the following Gram-Negative and Gram-positive bacteria and bacterial families and fungi: Actinomycetales (e.g., Corynebacterium, Mycobacterium, Norcardia), Cryptococcus neoformans, Aspergillosis, Bacillaceae (e.g., Anthrax, Clostridium), Bacteroidaceae, Blastomycosis, Bordetella, Borrelia (e.g., Borrelia burgdorferi), Brucellosis, Candidiasis, Campylobacter, Coccidioidomycosis, Cryptococcosis, Dermatocycoses, E. coli (e.g., Enterotoxigenic E. coli and Enterohemorrhagic E. coli), Enterobacteriaceae (Klebsiella, Salmonella (e.g., Salmonella typhi, and Salmonella paratyphi), Serratia, Yersinia), Erysipelothrix, Helicobacter, Legionellosis, Leptospirosis, Listeria, Mycoplasmatales, Mycobacterium leprae, Vibrio cholerae, Neisseriaceae (e.g., Acinetobacter, Gonorrhea, Menigococcal), Meisseria meningitidis, Pasteurellacea Infections (e.g., Actinobacillus, Heamophilus (e.g., Heamophilus influenza type B), Pasteurella), Pseudomonas, Rickettsiaceae, Chlamydiaceae, Syphilis, Shigella spp., Staphylococcal, Meningiococcal, Pneumococcal and Streptococcal (e.g., Streptococcus pneumoniae and Group B Streptococcus). These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis (e.g., mengitis types A and B), Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. Polynucleotides or polypeptides, agonists or antagonists of the invention, can be used to treat, prevent, and/or diagnose any of these symptoms or diseases. In specific embodiments, polynucleotides, polypeptides, agonists or antagonists of the invention are used to treat, prevent, and/or diagnose: tetanus, Diptheria, botulism, and/or meningitis type B.

Moreover, parasitic agents causing disease or symptoms that can be treated, prevented, and/or diagnosed by a polynucleotide or polypeptide and/or agonist or antagonist of the present invention include, but not limited to, the following families or class: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas and Sporozoans (e.g., Plasmodium virax, Plasmodium falciparium, Plasmodium malariae and Plasmodium ovale). These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), malaria, pregnancy complications, and toxoplasmosis. polynucleotides or polypeptides, or agonists or antagonists of the invention, can be used totreat, prevent, and/or diagnose any of these symptoms or diseases. In specific embodiments, polynucleotides, polypeptides, or agonists or antagonists of the invention are used to treat, prevent, and/or diagnose malaria.

Preferably, treatment or prevention using a polypeptide or polynucleotide and/or agonist or antagonist of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

### Chemotaxis

A polynucleotide or polypeptide and/or agonist or antagonist of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide and/or agonist or antagonist of the present invention may increase chemotaxic activity of particular cells. These chemotactic molecules can then be used to treat, prevent, and/or diagnose inflammation, infection, hyperproliferative diseases, disorders, and/or conditions, or any immune system disorder by increasing the number of cells targeted to a particular location in the body. For example, chemotaxic molecules can be used to treat, prevent, and/or diagnose wounds and other trauma to tissues by attracting immune cells to the injured location. Chemotactic molecules of the present invention can also attract fibroblasts, which can be used to treat, prevent, and/or diagnose wounds.

It is also contemplated that a polynucleotide or polypeptide and/or agonist or antagonist of the present invention may inhibit chemotactic activity. These molecules could also be used to treat, prevent, and/or diagnose diseases, disorders, and/or conditions. Thus, a polynucleotide or polypeptide and/or agonist or antagonist of the present invention could be used as an inhibitor of chemotaxis.

### Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors),or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

Additionally, the receptor to which a polypeptide of the invention binds can be identified by numerous methods known to those of skill in the art, for example, ligand panning and FACS sorting (Coligan, et al., Current Protocols in Immun., 1(2), Chapter 5, (1991)). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labeled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a CDNA library to identify the genes encoding the putative receptors.

Moreover, the techniques of gene-shuffling, motif-shuffling, exon-shuffling, and/or codon-shuffling (collectively referred to as “DNA shuffling”) may be employed to modulate the activities of polypeptides of the invention thereby effectively generating agonists and antagonists of polypeptides of the invention. See generally, U.S. Pat. Nos. 5,605,793, 5,811,238, 5,830,721, 5,834,252, and 5,837,458, and Patten, P. A., et al., Curr. Opinion Biotechnol. 8:724-33 (1997); Harayama, S. Trends Biotechnol. 16(2):76-82 (1998); Hansson, L. O., et al., J. Mol. Biol. 287:265-76 (1999); and Lorenzo, M. M. and Blasco, R. Biotechniques 24(2):308-13 (1998) (each of these patents and publications are hereby incorporated by reference). In one embodiment, alteration of polynucleotides and corresponding polypeptides of the invention may be achieved by DNA shuffling. DNA shuffling involves the assembly of two or more DNA segments into a desired polynucleotide sequence of the invention molecule by homologous, or site-specific, recombination. In another embodiment, polynucleotides and corresponding polypeptides of the invention may be altered by being subjected to random mutagenesis by error-prone PCR, random nucleotide insertion or other methods prior to recombination. In another embodiment, one or more components, motifs, sections, parts, domains, fragments, etc., of the polypeptides of the invention may be recombined with one or more components, motifs, sections, parts, domains, fragments, etc. of one or more heterologous molecules. In preferred embodiments, the heterologous molecules are family members. In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic(dpp), 60A, OP-2, dorsalin, growth differentiation factors (GDFs), nodal, MIS, inhibin-alpha, TGF-betal, TGF-beta2, TGF-beta3, TGF-beta5, and glial-derived neurotrophic factor (GDNF).

Other preferred fragments are biologically active fragments of the polypeptides of the invention. Biologically active fragments are those exhibiting activity similar, but not necessarily identical, to an activity of the polypeptide. The biological activity of the fragments may include an improved desired activity, or a decreased undesirable activity.

Additionally, this invention provides a method of screening compounds to identify those which modulate the action of the polypeptide of the present invention. An example of such an assay comprises combining a mammalian fibroblast cell, a the polypeptide of the present invention, the compound to be screened and 3[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of 3[H] thymidine in each case. The amount of fibroblast cell proliferation is measured by liquid scintillation chromatography which measures the incorporation of 3[H] thymidine. Both agonist and antagonist compounds may be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat, prevent, and/or diagnose disease or to bring about a particular result in a patient (e.g., blood vessel growth) by activating or inhibiting the polypeptide/molecule. Moreover, the assays can discover agents which may inhibit or enhance the production of the polypeptides of the invention from suitably manipulated cells or tissues. Therefore, the invention includes a method of identifying compounds which bind to the polypeptides of the invention comprising the steps of: (a) incubating a candidate binding compound with the polypeptide; and (b) determining if binding has occurred. Moreover, the invention includes a method of identifying agonists/antagonists comprising the steps of: (a) incubating a candidate compound with the polypeptide, (b) assaying a biological activity, and (b) determining if a biological activity of the polypeptide has been altered.

Also, one could identify molecules bind a polypeptide of the invention experimentally by using the beta-pleated sheet regions contained in the polypeptide sequence of the protein. Accordingly, specific embodiments of the invention are directed to polynucleotides encoding polypeptides which comprise, or alternatively consist of, the amino acid sequence of each beta pleated sheet regions in a disclosed polypeptide sequence. Additional embodiments of the invention are directed to polynucleotides encoding polypeptides which comprise, or alternatively consist of, any combination or all of contained in the polypeptide sequences of the invention. Additional preferred embodiments of the invention are directed to polypeptides which comprise, or alternatively consist of, the amino acid sequence of each of the beta pleated sheet regions in one of the polypeptide sequences of the invention. Additional embodiments of the invention are directed to polypeptides which comprise, or alternatively consist of, any combination or all of the beta pleated sheet regions in one of the polypeptide sequences of the invention.

### Targeted Delivery

In another embodiment, the invention provides a method of delivering compositions to targeted cells expressing a receptor for a polypeptide of the invention, or cells expressing a cell bound form of a polypeptide of the invention.

As discussed herein, polypeptides or antibodies of the invention may be associated with heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering polypeptides of the invention (including antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering polypeptides of the invention (e.g., polypeptides of the invention or antibodies of the invention) in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

### Drug Screening

Further contemplated is the use of the polypeptides of the present invention, or the polynucleotides encoding these polypeptides, to screen for molecules which modify the activities of the polypeptides of the present invention. Such a method would include contacting the polypeptide of the present invention with a selected compound(s) suspected of having antagonist or agonist activity, and assaying the activity of these polypeptides following binding.

This invention is particularly useful for screening therapeutic compounds by using the polypeptides of the present invention, or binding fragments thereof, in any of a variety of drug screening techniques. The polypeptide or fragment employed in such a test may be affixed to a solid support, expressed on a cell surface, free in solution, or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. One may measure, for example, the formulation of complexes between the agent being tested and a polypeptide of the present invention.

Thus, the present invention provides methods of screening for drugs or any other agents which affect activities mediated by the polypeptides of the present invention. These methods comprise contacting such an agent with a polypeptide of the present invention or a fragment thereof and assaying for the presence of a complex between the agent and the polypeptide or a fragment thereof, by methods well known in the art. In such a competitive binding assay, the agents to screen are typically labeled. Following incubation, free agent is separated from that present in bound form, and the amount of free or uncomplexed label is a measure of the ability of a particular agent to bind to the polypeptides of the present invention.

Another technique for drug screening provides high throughput screening for compounds having suitable binding affinity to the polypeptides of the present invention, and is described in great detail in European Patent Application 84/03564, published on Sep. 13, 1984, which is incorporated herein by reference herein. Briefly stated, large numbers of different small peptide test compounds are synthesized on a solid substrate, such as plastic pins or some other surface. The peptide test compounds are reacted with polypeptides of the present invention and washed. Bound polypeptides are then detected by methods well known in the art. Purified polypeptides are coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies capable of binding polypeptides of the present invention specifically compete with a test compound for binding to the polypeptides or fragments thereof. In this manner, the antibodies are used to detect the presence of any peptide which shares one or more antigenic epitopes with a polypeptide of the invention.

The human NFKB polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a NFKB polypeptide, or a bindable peptide fragment, of this invention, comprising providing a plurality of compounds, combining the NFKB polypeptide, or a bindable peptide fragment, with each of a plurality of compounds for a time sufficient to allow binding under suitable conditions and detecting binding of the NFKB polypeptide or peptide to each of the plurality of test compounds, thereby identifying the compounds that specifically bind to the NFKB polypeptide or peptide.

Methods of identifying compounds that modulate the activity of the novel human NFKB polypeptides and/or peptides are provided by the present invention and comprise combining a potential or candidate compound or drug modulator of calpain biological activity with an NFKB polypeptide or peptide, for example, the NFKB amino acid sequence as set forth in 109-118, 126, 128, 144-152, or 160-161, and measuring an effect of the candidate compound or drug modulator on the biological activity of the NFKB polypeptide or peptide. Such measurable effects include, for example, physical binding interaction; the ability to cleave a suitable calpain substrate; effects on native and cloned NFKB-expressing cell line; and effects of modulators or other calpain-mediated physiological measures.

Another method of identifying compounds that modulate the biological activity of the novel NFKB polypeptides of the present invention comprises combining a potential or candidate compound or drug modulator of a calpain biological activity with a host cell that expresses the NFKB polypeptide and measuring an effect of the candidate compound or drug modulator on the biological activity of the NFKB polypeptide. The host cell can also be capable of being induced to express the NFKB polypeptide, e.g., via inducible expression. Physiological effects of a given modulator candidate on the NFKB polypeptide can also be measured. Thus, cellular assays for particular calpain modulators may be either direct measurement or quantification of the physical biological activity of the NFKB polypeptide, or they may be measurement or quantification of a physiological effect. Such methods preferably employ a NFKB polypeptide as described herein, or an overexpressed recombinant NFKB polypeptide in suitable host cells containing an expression vector as described herein, wherein the NFKB polypeptide is expressed, overexpressed, or undergoes upregulated expression.

Another aspect of the present invention embraces a method of screening for a compound that is capable of modulating the biological activity of a NFKB polypeptide, comprising providing a host cell containing an expression vector harboring a nucleic acid sequence encoding a NFKB polypeptide, or a functional peptide or portion thereof (e.g., SEQ ID NOS:2); determining the biological activity of the expressed NFKB polypeptide in the absence of a modulator compound; contacting the cell with the modulator compound and determining the biological activity of the expressed NFKB polypeptide in the presence of the modulator compound. In such a method, a difference between the activity of the NFKB polypeptide in the presence of the modulator compound and in the absence of the modulator compound indicates a modulating effect of the compound.

Essentially any chemical compound can be employed as a potential modulator or ligand in the assays according to the present invention. Compounds tested as calpain modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel NFKB polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptides (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCTJUS96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a NFKB polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a NFKB polypeptide or peptide to measure a biological binding or ligand binding activity, the source may be a whole cell lysate that can be prepared by successive freeze-thaw cycles (e.g., one to three) in the presence of standard protease inhibitors. The NFKB polypeptide may be partially or completely purified by standard protein purification methods, e.g., affinity chromatography using specific antibody described infra, or by ligands specific for an epitope tag engineered into the recombinant NFKB polypeptide molecule, also as described herein. Binding activity can then be measured as described.

Compounds which are identified according to the methods provided herein, and which modulate or regulate the biological activity or physiology of the NFKB polypeptides according to the present invention are a preferred embodiment of this invention. It is contemplated that such modulatory compounds may be employed in treatment and therapeutic methods for treating a condition that is mediated by the novel NFKB polypeptides by administering to an individual in need of such treatment a therapeutically effective amount of the compound identified by the methods described herein.

In addition, the present invention provides methods for treating an individual in need of such treatment for a disease, disorder, or condition that is mediated by the NFKB polypeptides of the invention, comprising administering to the individual a therapeutically effective amount of the NFKB-modulating compound identified by a method provided herein.

### Antisense And Ribozyme (Antagonists)

In specific embodiments, antagonists according to the present invention are nucleic acids corresponding to the sequences contained in SEQ ID NO:1-108, 125, 127, 132-140, 158-159, or 264-284, or the complementary strand thereof. In one embodiment, antisense sequence is generated internally by the organism, in another embodiment, the antisense sequence is separately administered (see, for example, O'Connor, Neurochem., 56:560 (1991). Oligodeoxynucleotides as Antisense Inhibitors of Gene Expression, CRC Press, Boca Raton, FL (1988). Antisense technology can be used to control gene expression through antisense DNA or RNA, or through triple-helix formation. Antisense techniques are discussed for example, in Okano, Neurochem., 56:560 (1991); Oligodeoxynucleotides as Antisense Inhibitors of Gene Expression, CRC Press, Boca Raton, FL (1988). Triple helix formation is discussed in, for instance, Lee et al., Nucleic Acids Research, 6:3073 (1979); Cooney et al., Science, 241:456 (1988); and Dervan et al., Science, 251:1300 (1991). The methods are based on binding of a polynucleotide to a complementary DNA or RNA.

For example, the use of c-myc and c-myb antisense RNA constructs to inhibit the growth of the non-lymphocytic leukemia cell line HL-60 and other cell lines was previously described. (Wickstrom et al. (1988); Anfossi et al. (1989)). These experiments were performed in vitro by incubating cells with the oligoribonucleotide. A similar procedure for in vivo use is described in WO 91/15580. Briefly, a pair of oligonucleotides for a given antisense RNA is produced as follows: A sequence complimentary to the first 15 bases of the open reading frame is flanked by an EcoR1 site on the 5 end and a HindlIl site on the 3 end. Next, the pair of oligonucleotides is heated at 90° C. for one minute and then annealed in 2× ligation buffer (20 mM TRIS HCl pH 7.5, 10 mM MgCl2, 10 MM dithiothreitol (DTT) and 0.2 mM ATP) and then ligated to the EcoR1/Hind III site of the retroviral vector PMV7 (WO 91/15580).

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the antisense nucleic acid of the invention. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding a polypeptide of the invention, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature, 29:304-310 (1981), the promoter contained in the 3′ long terminal repeat of Rous sarcoma virus (Yamamoto et al., Cell, 22:787-797 (1980), the herpes thymidine promoter (Wagner et al., Proc. Natl. Acad. Sci. U.S.A., 78:1441-1445 (1981), the regulatory sequences of the metallothionein gene (Brinster et al., Nature, 296:39-42 (1982)), etc.

The antisense nucleic acids of the invention comprise a sequence complementary to at least a portion of an RNA transcript of a gene of interest. However, absolute complementarity, although preferred, is not required. A sequence “complementary to at least a portion of an RNA” referred to herein, means a sequence having sufficient complementarity to be able to hybridize with the RNA, forming a stable duplex; in the case of double stranded antisense nucleic acids of the invention, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a RNA sequence of the invention it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature, 372:333-335 (1994). Thus, oligonucleotides complementary to either the 5′ - or 3′ - non-translated, non-coding regions of a polynucleotide sequence of the invention could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′ -, 3′ - or coding region of mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double- stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci., 84:648-652 (1987); PCT'Publication NO: W088/09810, published Dec. 15, 1988) or the blood-brain barrier (see, e.g., PCT Publication NO: W089/10134, published Apr. 25, 1988), hybridization-triggered cleavage agents. (See, e.g., Krol et al., BioTechniques, 6:958-976 (1988)) or intercalating agents. (See, e.g., Zon, Pharm. Res., 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An a-anomeric oligonucleotide forms specific double-stranded hybrids with complementary RNA in which, contrary to the usual b-units, the strands run parallel to each other (Gautier et al., Nucl. Acids Res., 15:6625-6641 (1987)). The oligonucleotide is a 2-0-methylribonucleotide (Inoue et al., Nucl. Acids Res., 15:6131-6148 (1987)), or a chimeric RNA-DNA analogue (Inoue et al., FEBS Lett. 215:327-330 (1987)).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (Nucl. Acids Res., 16:3209 (1988)), methylphosphonate oligonucleotides can be prepared by use of controlled pore glass polymer supports (Sarin et al., Proc. Natl. Acad. Sci. U.S.A., 85:7448-7451 (1988)), etc.

While antisense nucleotides complementary to the coding region sequence of the invention could be used, those complementary to the transcribed untranslated region are most preferred.

Potential antagonists according to the invention also include catalytic RNA, or a ribozyme (See, e.g., PCT International Publication WO 90/11364, published Oct. 4, 1990; Sarver et al, Science, 247:1222-1225 (1990). While ribozymes that cleave mRNA at site specific recognition sequences can be used to destroy mRNAs corresponding to the polynucleotides of the invention, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature, 334:585-591 (1988). There are numerous potential hammerhead ribozyme cleavage sites within each nucleotide sequence disclosed in the sequence listing. Preferably, the ribozyme is engineered so that the cleavage recognition site is located near the 5′ end of the mRNA corresponding to the polynucleotides of the invention; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express the polynucleotides of the invention in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous messages and inhibit translation. Since ribozymes unlike antisense molecules, are catalytic, a lower intracellular concentration is required for efficiency.

Antagonist/agonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat, prevent, and/or diagnose the diseases described herein.

Thus, the invention provides a method of treating or preventing diseases, disorders, and/or conditions, including but not limited to the diseases, disorders, and/or conditions listed throughout this application, associated with overexpression of a polynucleotide of the present invention by administering to a patient (a) an antisense molecule directed to the polynucleotide of the present invention, and/or (b) a ribozyme directed to the polynucleotide of the present invention.

### Other Activities

The polypeptide of the present invention, as a result of the ability to stimulate vascular endothelial cell growth, may be employed in treatment for stimulating re-vascularization of ischemic tissues due to various disease conditions such as thrombosis, arteriosclerosis, and other cardiovascular conditions. These polypeptide may also be employed to stimulate angiogenesis and limb regeneration, as discussed above.

The polypeptide may also be employed for treating wounds due to injuries, burns, post-operative tissue repair, and ulcers since they are mitogenic to various cells of different origins, such as fibroblast cells and skeletal muscle cells, and therefore, facilitate the repair or replacement of damaged or diseased tissue.

The polypeptide of the present invention may also be employed stimulate neuronal growth and to treat, prevent, and/or diagnose neuronal damage which occurs in certain neuronal disorders or neuro-degenerative conditions such as Alzheimer's disease, Parkinson's disease, and AIDS-related complex. The polypeptide of the invention may have the ability to stimulate chondrocyte growth, therefore, they may be employed to enhance bone and periodontal regeneration and aid in tissue transplants or bone grafts.

The polypeptides of the present invention may be employed to stimulate growth and differentiation of hematopoietic cells and bone marrow cells when used in combination with other cytokines.

The polypeptide of the invention may also be employed to maintain organs before transplantation or for supporting cell culture of primary tissues.

The polypeptide of the present invention may also be employed for inducing tissue of mesodermal origin to differentiate in early embryos.

The polypeptide or polynucleotides and/or agonist or antagonists of the present invention may also increase or decrease the differentiation or proliferation of embryonic stem cells, besides, as discussed above, hematopoietic lineage.

The polypeptide or polynucleotides and/or agonist or antagonists of the present invention may also be used to modulate mammalian characteristics, such as body height, weight, hair color, eye color, skin, percentage of adipose tissue, pigmentation, size, and shape (e.g., cosmetic surgery). Similarly, polypeptides or polynucleotides and/or agonist or antagonists of the present invention may be used to modulate mammalian metabolism affecting catabolism, anabolism, processing, utilization, and storage of energy.

Polypeptide or polynucleotides and/or agonist or antagonists of the present invention may be used to change a mammal's mental state or physical state by influencing biorhythms, caricadic rhythms, depression (including depressive diseases, disorders, and/or conditions), tendency for violence, tolerance for pain, reproductive capabilities (preferably by Activin or Inhibin-like activity), hormonal or endocrine levels, appetite, libido, memory, stress, or other cognitive qualities.

Polypeptide or polynucleotides and/or agonist or antagonists of the present invention may also be used as a food additive or preservative, such as to increase or decrease storage capabilities, fat content, lipid, protein, carbohydrate, vitamins, minerals, cofactors or other nutritional components.

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

Polypeptide or polynucleotides and/or agonist or antagonists of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. Such a use may be administered either prior to an extraterrestrial event, during an extraterrestrial event, or both. Moreover, such a use may result in a number of beneficial changes in the recipient, such as, for example, any one of the following, non-limiting, effects: an increased level of hematopoietic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoietic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid the recipient by decreasing their basal daily energy requirements; (ii) effectively lower the level of oxidative and/or metabolic stress in recipient (i.e., to enable recipient to cope with increased extraterrestial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Also preferred is a method of treatment of an individual in need of an increased level of a protein activity, which method comprises administering to such an individual a pharmaceutical composition comprising an amount of an isolated polypeptide, polynucleotide, or antibody of the claimed invention effective to increase the level of said protein activity in said individual.

Having generally described the invention, the same will be more readily understood by reference to the following examples, which are provided by way of illustration and are not intended as limiting.

### EXAMPLES

### Example 1

### Method of Creating the NFkB Subtraction Library

### Cell Culture

For the subtraction library, duplicate flasks of THP-1 cells (108) were cultured at 10⁶/ml in RPMI containing 10% heat inactivated fetal calf serum, 2 mM L-glutamine with either medium, or with BMS-205820 (2 uM) for 30 minutes at 37° C. in 5% CO₂. LPS (100 ng/ml) was added to both groups and the cells were cultured for an additional 2 hours. At the end of the incubation, cells were pelleted, washed one time with 10 ml PBS, and stored at −80° C.

For the microarray procedure, 10⁸ THP-1 cells were cultured at 10⁶/ml as above with either medium, BMS-205820 (2 uM), or dexamethasone (1 uM) for 30 minutes at 37° C. in 5% CO₂. LPS (100 ng/ml) was added to each group, and the incubation continued for an additional two hours. At the end of this incubation, cells were pelleted, washed one time with 10 ml PBS, and stored at −80° C.

### RNA Isolation

Poly A+ mRNA was isolated using the FastTrack 2.0 kit (Invitrogen, Carlsbad, Calif.) according to manufacturer's instructions.

### Construction of the Subtraction Library

For first strand synthesis, Oligo d(t) Not (5′-AAGCAGTGGTAACAACGCAGAGTGCGGCCGA(T)₁₅A/G-3′ (SEQ ID NO: 119)) and CapSal (5′-AAGCAGTGGTAACAACGCAGAGTCGACrGrGrG-3′ (SEQ ID NO:120)) primers were added to the RNA, and incubated for 2 minutes at 72° C., followed by 2 minutes on ice. The reaction was initiated with dNTPs and SuperScript II (Life Technologies, Baltimore, Md.). The second strand was synthesized using KlenTaq (Clontech, Palo Alto, Calif.), dNTPs, and primer (5′-AAGCAGTGGTAACAACGCAGAGTCGAC-3′ (SEQ ID NO:121)). The reaction was purified using a Microspin S-40010 HR column (Amersham Inc., Chicago, Ill.), and double digested with Not I and Sal I. The digested products were size fractionated using a ChromaSpin 100 column (Clontech).

The digested cDNA from the LPS group (tester) was cloned into the vector pSPORT1 precut with Not I and Sal I. The digested cDNA from the LPS plus BMS-205820 group (driver) was cloned into the pSPORT2 vector that was also cut with Not I and Sal I. The tester cDNA library in pSPORT 1 was electroporated into DH12S cells for single strand DNA isolation, and the driver cDNA library was electroporated into DH10B cells. The primary transformants were amplified in semi-solid agar.

Single stranded cDNA from the tester pSPORT1 library was rescued using M13K07 helper phage. DNA was isolated from the amplified driver pSPORT2 library using a Qiagen maxi-prep plasmid kit. The driver library was linearized using Sal I and reverse transcribed with T7 RNA polymerase, rNTPs, and biotin-16-UTP. The biotinylated RNA was treated with RNAse-free DNAse, precipitated, and purified using G-50 spin columns (Bio-Rad, Hercules, Calif.).

Prior to hybridizing the single stranded DNA with the biotinylated RNA, the poly dA region of the single stranded DNA was blocked using a d(T)-Not I oligonucleotide, dTTP nucleotides, and Taq polymerase. The single stranded cDNA was further blocked using Cot-1 DNA (Life Technologies).

For the subtractive hybridization, 600 ng of single stranded tester cDNA (poly dA, Cot-1 blocked pSPORTI) and 80 ug biotinylated driver RNA were used. The biotinylated driver RNA was incubated with hybridization buffer (40% formamide, 50 mM HEPES, 1 mM EDTA, 0.1% SDS) at 65° C. for 10 minutes, followed by I minute at 4° C. After this incubation, the tester cDNA was added and the sample was incubated for 24 hours at 42° C. Hybrids were removed by addition of streptavidin followed by phenol/chloroform extractions. The remaining single stranded DNA was precipitated, and used in repair reactions.

The single stranded DNA was repaired using T7 pSPORT primer, dNTPs and Precision-Taq polymerase. The repaired DNA was electroporated into DH12S cells, and then amplified to generate single stranded DNA for a second round of subtraction with the biotinylated driver RNA.

### Example 2

### Method of Identifying Differentially Expressed NFkB Modulated Genes Using Microarray Methodology

Colony purified, sequence verified clones were obtained from Research Genetics. Inserts were PCR amplified, purified by silica binding using MAFB N0B glass filter plates (Millipore), dried and resuspended in 50% DMSO. A Gen III micrarray spotter (Molecular Dynamics, Sunnyvale, Calif.) was used to array the PCR products onto glass slides. The slides (5 GAPS, Amersham) were washed for 5 minutes in 80° C. water before spotting. After spotting, the slides were dried at 50% humidity for two hours, UV crosslinked (50 millijoules), and baked for one hour in a vacuum oven.

Probes were synthesized by reverse transcribing RNA isolated from each of the treatment groups. For each group, reactions contained 1 ug poly A+ mRNA, 0.5 ug [CGA] anchored Oligo dT (25), and RNAse free water. Following a five minute incubation at 70° C., and a ten minute incubation at room temperature, SuperScript II reverse transcriptase (Life Technologies), DTT, dNTPs, and Cy3-dCTP were added. The reaction was incubated for 90 minutes at 42° C., followed by purification over GFX columns (Pharmacia Biotech Inc, Piscataway, N.J.) according to manufacturer's instructions. The eluate was dried in a speedvac, and resuspended in Hybridization buffer (50% formamide, 1× Amersham Hyb Version 2 buffer, and 2.5 ug Poly-A (80). The samples were incubated for 30 minutes at 70° C., followed by 10 minutes at room temperature. The probe mix was added to each microarray slide, covered, and incubated at 42° C. overnight in a humid chamber. Duplicate slides were probed for each group.

The slides were washed by shaking gently at 32° C. in 1×SSC/0.2% SDS for 5 minutes. The slides were then shaken gently for 10 minutes in 0.1×SSC/0.2% SDS. The slides were dipped quickly in water, dried with compressed air, and stored in the dark at room temperature until analysis.

The slides were scanned in a Molecular Dynamics GenIII scanner using Cy3 emission filters. The image files obtained were analyzed by integrating the spot values using the Arrayvision (Imaging Research Inc., Saint Catharines, Ontario) software. The values for each spot were normalized by the median value for all spots in an image. A median and median average deviation was determined using four replicate spots (duplicate spots on duplicate slides) for each gene analyzed.

The NFkB associated clones that were identified by microarray methodology are summarized in Table III and IV herein.

### Example 3

### Expression Profiling of the Novel NFkB Associated Polypeptides

A number of methods may be employed to identify the tissue expression profile of the NFkB associated polypeptides of the present invention. Once exemplary method would be to measure the steady state mRNA levels of the NFkB associated sequences using quantitative PCR. A PCR primer pair corresponding to one of the polynucleotide sequences provided in Table I, II, III, and/or IV could be designed. Such primers would preferably be at least 17 bp in length and correspond to non-repetitive elements within the target sequence. Briefly, first strand cDNA can be made from commercially available mRNA (Clontech) and subjected to real time quantitative PCR using a PE 5700 instrument (Applied Biosystems, Foster City, Calif.) which detects the amount of DNA amplified during each cycle by the fluorescent output of SYBR green, a DNA binding dye specific for double strands. The specificity of the primer pair for its target could be verified by performing a thermal denaturation profile at the end of the run which gives an indication of the number of different DNA sequences present by determining melting Tm. In the case of the NFkB associated sequence primer pair, experiments resulting in only a single DNA fragment representing the presence of a homogeneous melting point would be utilitzed. Contributions of contaminating genomic DNA to the assessment of tissue abundance would be controlled for by performing the PCR with first strand made with and without reverse transcriptase. In all cases, the contribution of material amplified in the no reverse transcriptase controls should be negligible.

Small variations in the amount of cDNA used in each tube could be determined by performing a parallel experiment using a primer pair for a gene expressed in equal amounts in all tissues, cyclophilin. Such data could be used to normalize the data obtained with the NFkB associated sequence primer pair. The PCR data could then be converted into a relative assessment of the difference in transcript abundance amongst the tissues tested and the data are presented in bar graph form.

Nonetheless, the following methods were used to assess the expression profile of the NFkB assocaited polypeptides of the present invention. Briefly, poly (A)⁺ mRNA was isolated from THP-1 cells that were either unstimulated, or stimulated with 100 ng/ml LPS for two hours in the presence and absence of BMS-205820 (2 uM) using the Fast Track isolation kit (Invitrogen, Carlsbad, Calif.) according to the manufacturer's instructions. RNA quality and quantity were evaluated using UV spectrometry and capillary electrophoresis with the RNA 6000 Assay (Agilent). Five-hundred nanograms of polyA RNA was used for first-strand cDNA synthesis using the SuperScriptTm First-Strand Synthesis System for RT-PCR (Invitrogen) according to the manufacturer's instructions with 250 ng of random hexamers.

PCR reactions were performed in a total volume of 40 ul containing master mix (SYBR Green I dye, 50 mM Tris-Cl pH 8.3, 75 mM KCl, DMSO, Rox reference dye, 5 mM MgCl₂, 2 mM dNTP, I unit Platinum Taq High Fidelity enzyme), 0.5 uM each of forward and reverse gene-specific primers, and cDNA (8 ul of a 1:36 dilution of the first strand reaction mix). For tissue expression analyses, PCR reactions included 2 ul of cDNA derived from the Human Multiple Tissue cDNA panel I and Human Immune System MTC Panel (Clontech, Palo Alto, Calif.). The amplification program consisted of a 10 minute incubation at 95° C., followed by 40 cycles of incubations at 95° C. for 15 seconds, 60° C. for 1 minute. The amplification was followed by melting curve analysis at 60° C. to determine the specificity of the amplification reaction. A negative control without cDNA template was run to assess the overall specificity. The data were analyzed using the TaqMan 5700 software with the threshold value set to 0.5. The message levels of GAPDH were used to normalize the amounts of cDNA for each reaction.

Gene specific primers were designed using the Primer Express software and synthesized by Sigma Genosys (The Woodlands, Tex.). Primer names and sequences are below:

| Primer Name | Primer Sequence | SEQ ID NO: |
|---|---|---|
| CyclinLF | GCTTGCATCTACCTTGCAGCTA | 164 |
| CyclinLR | ACGAGTTGGCAACGGAATCT | 165 |
| AD037F | CCATTCAGAAGTCGGAGCTCTTAG | 162 |
| AD037R | GAAGCTCTTGCCCTCATGGTA | 163 |
| HGAPDH-F3 | AGCCGAGCCACATCGCT | 166 |
| HGAPDH-R1 | GTGACCAGGCGCCCAATAC | 167 |
| AP002338F2 | GGTCTTTCCTCCAGTGTCACAATA | 206 |
| AP002338R2 | GAACCTCCTACCTTTCAGGCACTA | 207 |
| AL136163F | CACATGCAACATTTGGATTCAGT | 208 |
| AL136163R | ACGGTTACTTTCTTGTGAGTCTTTGA | 209 |
| AC008435F2 | GAGACAATGCGAATGCAAAGAG | 210 |
| AC008435R2 | CCACCATATCTGACCCAAGAGAGT | 211 |
| 346607F2 | GGAAGGATGAAGCGGAGAAAGT | 212 |
| 346607R2 | GACTGAGTCCAGAGAAATGTGTGAA | 213 |
| 337323F | GGCTGGCAATTCGAAAGGA | 214 |
| 337323R | GGAATCACCATCAGCTTGTTTAGC | 215 |
| AL354881F | GGTCCTTGATGTCGATATTCTTAACAC | 216 |
| AL354881R | CCATGCTTTAGTTGCCATTTACTTCT | 217 |
| 127F | TTGCAAGTCTTGGATGTGGTTT | 218 |
| 127R | CTGGCACGTAATGGTCACTGTT | 219 |
| 7248F2 | GCTGATGGAAGGGAGTCAACA | 220 |
| 7248R2 | CTCCATAAGGGAGCTCACCTACTT | 221 |
| 404343F | GTGGTACAGTGCAATGTCTTCCAT | 222 |
| 404343R | CATGACCTTTGCAAGACCTCCTA | 223 |
| AC015564F | CCACAGTAGCCATGGGTCAAT | 224 |
| AC015564R | CTATGGCAGGGCTTGGACAA | 225 |
| 242250F | CCTGGCAGATTTGCATGACA | 226 |
| 242250R | CAAGTGGAAGGAAGAGCAATCAA | 227 |
| AC024191F | GGCGTCTTCATTCGCTACAAA | 228 |
| AC024191R | ACAGGGAAACCTTCACAATGTAGTC | 229 |
| 204305F | CCATCAGCACGTTTGGAGTGT | 230 |
| 204305R | CTAGCCCACCAGCATCCATT | 231 |
| 235347F2 | CCTCAACAGCAACATCTCATCAG | 232 |
| 235347R2 | CCCACAGCTTCTGGTTTTGAC | 233 |
| AC005625F | GCTCAGGAGGCCAGACTATTCA | 234 |
| AC005625R | TGGAGTGCAGTGGTGTGATCA | 235 |
| AC007014F | CCTTTGGAGGTGATGTCATTGA300 | 236 |
| AC007014R | TGCGCTCTTGGAGTTTCCTACT | 237 |
| AC010791F2 | GGGAACAGATTGCTCCATGGT | 238 |
| AG010791R2 | TGCATTGACGCTAGGAAGAAAG | 239 |
| AC023602F | TGTGGGACCAGAGGAAGAAATG | 240 |
| AC023602R | CAACCCATAGTTTTGCTGAGTCAT | 241 |
| AC008576F | GAAGGGTGGAGGTGGATGAA | 242 |
| AC008576R | CACGCAAGTCCCTAAGCTGTAA | 243 |
| AL158062F2 | GAGTACAGCAACAGTGGCTCCAT | 244 |
| AL158062R2 | CAGCTAGCATCCATCCCATCA | 245 |
| 116917F | GAAGGTCACACCCTCTGGTCTT | 246 |
| 116917R | TGGATGCCGTCAATTCAGATT | 247 |
| 1137189F | GCCTCGTCCTTCACCATTTGT | 248 |
| 1137189R | GGATTTCCAGCCTCATCTTAACA | 249 |
| 899587F | CCCAACCAAACAAAGACAGTTACTC | 250 |
| 899587R | CCTTTTCCTTTCCTGCACACA | 251 |
| 30507F | CCATTGCTCAGTGGATGTTCA | 252 |
| 30507R | GGGAGGCTGAGGAATTTGAGT | 253 |
| AC040977F | GGGCTCTTAGTATCGGAGGATTG | 254 |
| AC040977R | CCCAACACAGGAGAGACTAAGGA | 255 |
| AG012357F2 | CTGATTGTGCACCTGTGGTTAAA | 256 |
| AG012357R2 | GAGGGCAGATGCTGTCTAAACAT | 257 |
| 360F | GCCTAGCCTTGTGTGGAATTC | 258 |
| 360R | ACCCTAGGATCCCAGAAAGCA | 259 |
| AC025631F | GGTGGAGGATAAGCAAGAGCATA | 260 |
| AC025631R | CATCTTGGTCTTCTGGCTCATTT | 261 |
| 262F | CATGATTGAGGGCTTGGTGTT | 262 |
| 262R | CCAGTCATAAGCAAGCCTGTCA | 263 |

### Example 4

### Method of Assessing the Expression Profile of the Novel NFkB Associated Polypeptides in Primary Cell Lines

The expression profile of each NFkB associated polypeptide may be obtained by isolating mRNA from specific cell lines, either under control, or treated conditions, and subjecting the mRNA to quantitative RT-PCR reactions. The RT-PCR conditions may be essentially as described in Example 3, or as otherwise described herein or known in the art. Some representative cell lines and conditions are provided below.

### THP-1 Human Monocyte Lines

The THP-1 human monocyte line was stimulated with 100 ng/ml LPS, 10 ng/ml TNFα (Peprotech, Rocky Hill, N.J.), or 100 units/ml interferon-γ (IFN-γ, Peprotech) for either 8, 24, or 48 hours. Controls were cultured with medium alone. Following stimulation, mRNA was isolated from the cultured cell line, and used to prepare cDNA as described in Example 3. The levels of each NFkB associated polypeptide mRNA may be measured by RT-PCR analysis using the primers described herein for each gene. The values are normalized to the GAPDH housekeeping gene.

In the case of the AD037 NFkB associated polypeptide, the same primer pairs described in Example 3 were used for RT-PCR (SEQ ID NO:162 and 163). As shown in FIG. 18, AD037 mRNA was upregulated in THP-1 cells in response to stimuli that activate the NF-kB pathway including LPS and TNFα. Little upregulation was observed in response to IFN-γ, which fails to activate the NF-KB pathway.

### Human Peripheral Blood Neutrophils

Human peripheral blood neutrophils were isolated from two different donors by differential centrifugation through ficoll, followed by sedimentation through dextran sulfate. The cells were stimulated for 24 or 48 hours with 100 ng/ml LPS. Controls were cultured with medium alone. Following stimulation, nRNA was isolated from the cultured cell line, and used to prepare cDNA as described in Example 3. The levels of each NFkB associated polypeptide mRNA may be measured by RT-PCR analysis using the primers described herein for each gene. The values are normalized to the GAPDH housekeeping gene.

In the case of the AD037 NFkB associated polypeptide, the same primer pairs described in Example 3 were used for RT-PCR (SEQ ID NO:162 and 163). As shown in FIG. 19, AD037 was also strongly upregulated in human peripheral blood neutrophils in response to LPS stimulation.

### Synovial Fibroblasts

Synovial fibroblasts were obtained from Cell Applications, INC. (San Diego, Calif.), and cultured for either 1, 6, or 24 hours with TNFα (10 ng/ml), IL-1α (10 ng/ml, Peprotech), IL-17 (10 ng/ml, R&D Systems, Minneapolis, Minn.), or IL-17B-Ig fusion protein (5 ng/ml). The IL-17B protein was produced by fusing the full length IL-17B sequence (Shi et al. (2000) J. Biol. Chem. 275:19167-19176) to the human IgG1 Fc region. Controls were cultured with medium alone. Following stimulation, mRNA was isolated from the cultured cell line, and used to prepare cDNA as described in Example 3. The levels of each NFkB associated polypeptide mRNA may be measured by RT-PCR analysis using the primers described herein for each gene. The values are normalized to the GAPDH housekeeping gene.

In the case of the AD037 NFkB associated polypeptide, the same primer pairs described in Example 3 were used for RT-PCR (SEQ ID NO: 162 and 163). As shown in FIG. 20, AD037 mRNA was selectively upregulated in synovial fibroblasts in response to IL-17B. No upregulation was observed in response to IL-1α, TNF-α, or IL-17.

### Human Peripheral Blood B Cells

Human peripheral blood B cells were isolated from one donor by centrifugation through ficoll followed by T cell removal. B cells were stimulated for 6 or 24 hours with 2.4 micrograms/ml anti-CD40 antibody. Controls were cultured with medium alone. Following stimulation, mRNA was isolated from the cultured cell line, and used to prepare cDNA as described in Example 3. The levels of each NFkB associated polypeptide mRNA may be measured by RT-PCR analysis using the primers described herein for each gene. The values are normalized to the GAPDH housekeeping gene.

In the case of the AD037 NFkB associated polypeptide, the same primer pairs described in Example 3 were used for RT-PCR (SEQ ID NO:162 and 163). As shown in FIG. 21, AD037 mRNA was induced in response to CD40 crosslinking in human peripheral blood B cells, another pathway known to activate NF-kB

### Example 5

### Method of Assessing Effect of Overexpressing the NFkB Associated Polypeptides of the Present Invention on the Level of TNF-Alpha Secretion

THP-1 cells (10⁷/group) were electroporated with 20 ug of either pcDNA3.1 mychis (Invitrogen), or pcDNA3. 1 mychis with the encoding sequence of a full length NF-kB associated polynucleotide of the present invention (e.g., cyclin L, AD037, etc.). All groups also included 5 ug of CMV-β-galactosidase to control for differences in transfection efficiency. Cells were electroporated in serum-free RPMI 1640 with 975 uFd and 320 volts. Following electroporation, cells were pelleted, and resuspended in 10 ml RPMI containing 10% FBS. Cells were cultured for 48 hours at 37° C., and then harvested for stimulation. A fraction of cells (10%) from each culture was stained for LacZ expression to estimate transfection efficiency. All groups had similar efficiencies, approximately 20%. The remainder of cells from each group were stimulated for 6 hours with 100 ng/ml LPS. At the end of the stimulation, supernatants were collected and analyzed for TNFα by ELISA (Pharmingen, San Diego, Calif.).

### Example 6

### Method of Creating Expression Vectors and Mutant Constructs for AD037 NFkB Associated Polypeptide of the Present Invention

### AD037 Expression Vector and Mutant Constructs

The sequence encoding full length, wild type AD037 (SEQ ID NO:125) was amplified by PCR and cloned into pcDNA3.1 (Invitrogen) containing an amino terminal FLAG tag. This vector was used as a template for the transformer site-directed mutagenesis protocol (Clontech). Briefly, the plasmid was denatured followed by annealing of the mutagenic and selection primers. The mutant strand was synthesized by addition of T4 DNA polymerase and T4 DNA ligase. The parental DNA was selectively linearized using MfeI, the site mutated in the selection primer. The digested DNA was transformed into a repair-deficient E. coli strain (mutS). Plasmid DNA was isolated from selected colonies and subjected to another round of digestion with MfeI to completely remove parental DNA. The digested DNA was transformed into E. coli; selected colonies were amplified; plasmid DNA was isolated and sequenced.

### Cloning Primers

| | |
|---|---|
| 5′ with EcoRI site: | |
| 5′GAATTCTTGTCTGCAGACAAGAGGAAGAG3′ | (SEQ ID NO:303) |
| 3′ with NotI site: | |
| 5′GCGGCCGCTTACTTGGCCTCCACCAGCTG3′ | (SEQ ID NO:304) |

### Primers for Mutagenesis

| | | |
|---|---|---|
| Selection primer: | 5′GCTTGACCGACAGTTGCATGAAG3′ | (SEQ ID NO:305) |
| 409 (Δmyr): | 5′GAAGTCGGAGCTCTTAAACTGCTACCATGAGG3′ | (SEQ ID NO:306) |
| 410 (Δras): | 5′TTCTCTATCAACGGCGTGGAAGTCCCCCAT3′ | (SEQ ID NO:307) |

### Example 7

### Method of Expressing the NFkB Associated Polypeptides of the Present Invention in Mammalian Cells for Western Blot or Confocal Microscopy

Cos7 cells were transfected using Lipofectamine PLUS reagent (Invitrogen) with 5 ug of either the pcDNA3.1 vector alone, or the pcDNA3.1 containing the full-length encoding region of a NFkB associated polypeptide of the present invention operably linked to the Flag epitope tag. After resting for 24 hours, the cells were harvested using trypsin, washed with PBS, and either lysed in RIPA buffer (10 mM sodium phosphate pH 7.2, 0.25 M sodium chloride, 0.1% SDS, 1% NP40, 1% sodium deoxycholate, 2 mM EDTA, protease inhibitor cocktail) for Western blot analysis, or fixed in 1% paraformaldehyde for confocal analysis.

For Western blotting, whole cell lysates were electrophoresed through 4-20% Tris-glycine gels (Novex, San Diego, Calif.), transferred to nitrocellulose, and blocked overnight in 5% BSA in Tris buffered saline. Blots were probed with a mouse monoclonal IgG specific for the Flag epitope tag (Sigma, St. Louis, Mo.), followed by detection with HRP-conjugated antibodies specific for mouse IgG, and ECL (Amersham Pharmacia Biotech, Piscataway, N.J.).

In the case of the Western blot provided in FIG. 86 specifically, THP-1 monocytes were stimulated with LPS (100 ng/ml) in the presence and absence of BMS-205820 (pep) for 4 to 24 hours. At each time point, cells were harvested and lysed in RIPA buffer as described. Whole cell lysates were electrophoresed through a 4-20% Tris-glycine gel, transferred to nitrocellulose, blocked overnight with 5% non fat dry milk in Tris-buffered saline, and probed with rabbit antisera raised to a peptide containing amino acids 11-24 of AD037 (SEQ ID NO:289). The immunizing peptide contained an additional cysteine at the N-terminus to facilitate conjugation to KLH for injection into rabbits. Bands were detected with HRP-tagged anti-rabbit antibodies followed by ECL as shown in FIG. 86. The arrow in FIG. 86 indicates a specific band that is blocked when the rabbit antisera is preincubated with immunizing peptide and which correspondes to the expressed AD037 polypeptide.

For confocal analysis, cells were fixed for 20 minutes on ice. The cells were incubated with 1 ug mouse IgG₁ specific for Flag in 50 ul staining buffer (0.1% saponin, 5 mg/ml BSA in PBS) for 30 minutes on ice. The cells were washed two times with PBS containing 2% FBS, and then incubated with FITC-conjugated antibodies specific for mouse IgG (Jackson ImmunoResearch, West Grove, Pa.) in staining buffer for 30 minutes on ice. The cells were washed two times, resuspended in PBS/FBS, and analyzed with a BioRad MRC1024 confocal microscope. Negative controls were stained with secondary antibody alone.

In the case of the confocal analysis provided in FIG. 87 specifically, Cos cells were transfected with either vector containing a FLAG epitope tag, or a FLAG-tagged vector encoding either wild type AD037, or AD037 with the myristoylation site deletion (AD037Δmyr), or AD037 with a deletion of the Ras association motif (AD037Δras). Transfectants were permeabilized, and stained with mouse monoclonal antibodies specific for FLAG. The antibodies were detected with FITC-anti-mouse secondary antibodies and visualized on the confocal microscope as shown in FIG. 87.

### Example 8

### Method of Identifying a Binding Partner of the NFkB Associated Polypeptides of the Present Invention Using the Yeast Two Hybrid System

A library was generated using a ZAP-cDNA synthesis kit (Stratagene) in the vector pJG4.5 (Mendelshohn et al. (1994) Curr. Opin. Biotechnol. 5:482-486). The cDNA was generated from poly (A)⁺ mRNA isolated from THP-1 cells stimulated for two hours with 100 ng/ml LPS (S. typhosa 0901, Sigma, St. Louis, Mo.). The bait constructs used for screening the library were generated by fusing the full length encoding polynucleotide sequence of a NFkB associated polypeptide of the present invention (e.g., AD037, or cyclinL) to the DNA binding and dimerization domains of the bacterial repressor Lex A in the vector pJK202 (Mendelshohn et al. (1994) Curr. Opin. Biotechnol. 5:482-486). These baits were transformed into the yeast strain EGY48, which harbors reporter plasmids containing 6 Lex A operators upstream of the leu2 gene, and 8 Lex A operators upstream of the lacZ gene (Estojak et al. (1995) Mol. Cell. Biol. 15:5820-5829). On their own, the NFkB associated polypeptide fusions constructs (e.g., AD037, or cyclinL) failed to activate either reporter. The EGY48 strains containing the baits were transformed with 1 ug of the THP-1 cDNA library using lithium acetate (Clontech, Palo Alto, Calif.). Approximately 100 interacting clones were selected for each bait based on their ability to grow on medium lacking leucine, as well as by LacZ activity. Individual library plasmids were isolated by transforming KC8 bacteria carrying trpC, leuB, and hisB mutations with DNA isolated from positive yeast colonies, and selecting on medium lacking tryptophan. Plasmids encoding the interactors were isolated and sequenced. The isolated plasmids were transformed into EGY48 strains harboring unrelated bait plasmids including the S. cerevisiae RNA polymerase and NF-kappaB p50 Rel domain (amino acids 245-367) to test the specificity of the interactions.

### Example 9

### Method of Assessing Additional Expression Profiles of the Novel NFkB Associated Polypeptides in Primary Cell Lines

Expression profiling was also performed using quantitative RT-PCT using Taqman analysis. Specifically, the following was performed for the AD037 NFkB associated polypeptide of the present invention, although the same assays could be applied to the other NFkB associated polypeptides of the present invention using the primer pairs provided herein, as applicable.

PolyA+ mRNA was isolated from THP1 cells that were either unstimulated, stimulated with LPS for 2 hours, or stimulated with LPS for 2 hours in the presence of the peptide BMS-205820 (2 EM). In some experiments, THP-1 cells were stimulated with LPS in the presence of the glucocorticoid dexamethasone (100 nM), and the IKK-2 inhibitor, BMS-345541 (10 μM). RNA quality and quantity were evaluated using UV spectrometry and capillary electrophoresis with the RNA 6000 Assay by Agilent. Five-hundred nanograms of polyA RNA was used for first-strand cDNA synthesis using the SuperScript™ First-Strand Synthesis System for RT-PCR (Life Technologies) following the manufacturer's instructions with 250 ng of random hexamers.

For the NF-KB knockout studies, wild type 3T3 cells, 3T3 fusions of embryonic fibroblasts derived from p65 knockouts, or embryonic fibroblasts derived from p50 and RelB knockouts were stimulated for 2 hours with 10 ng/ml TNFα or 10 ng/ml PMA. RNA isolation and cDNA synthesis were performed as described above.

PCR Reactions were performed in a total volume of 40 μl. The master mix contained SYBR Green I Dye, 50 mM Tris-HCl pH8.3, 75 mM KCl, DMSO, Rox reference dye, 5 mM MgCl₂, 2 mM dNTP, Platinum Taq High Fidelity (1U/reaction), and 0.5 μM of each primer. The cDNA was diluted 1:36 from the synthesis reaction and eight microliters was used in each PCR reaction. The amplification program consisted of a 10 minute incubation at 95° C. followed by forty cycles of incubations at 95° C. for 15 seconds and 60° C. for 1 minute. Amplification was followed by melting curve analysis at 60° C. to demonstrate that the amplification was specific to a single amplicon. A negative control without cDNA template was run to assess the overall specificity.

A relative value for the initial target concentration in each reaction was determined using the TaqMan 5700 software. The threshold value was set to 0.5 to obtain cycle threshold values that were used to assign relative message levels for each target. The message levels of GAPDH were determined for each cDNA sample and were used to normalize all other genes tested from the same cDNA sample.

### Primers

| | |
|---|---|
| Mouse AD037 | |
| F5′ CCTATGGGTCTGTGACCAACGT 3′ | (SEQ ID NO:285) |
| Mouse AD037R | |
| 5′ CCATCTTCTACCCGGAACTTGT 3′ | (SEQ ID NO:286) |
| Mouse GAPDHF | |
| 5′ CATGGCCTTCCGTGTTCCTA 3′ | (SEQ ID NO:287) |
| Mouse GAPDHR | |
| 5′ CCTGCTTCACCACCTTCTTGA 3′ | (SEQ ID NO:288) |
| AD037F | |
| 5′ CCATTCAGAAGTCGGAGCTCTTAG 3′ | (SEQ ID NO:162) |
| AD037R | |
| 5′ GAAGCTCTTGCCCTCATGGTA 3′ | (SEQ ID NO:163) |
| hGAPDH-F3 | |
| 5′ AGCCGAGCCACATCGCT 3′ | (SEQ ID NO:166) |
| hGAPDH-R1 | |
| 5′ GTGACCAGGCGCCCAATAC 3′ | (SEQ ID NO:167) |

The results of the additional expression profile with LPS, Dexamethosone, and the IKK-2 inhibitor BMS-345541 for AD037 are provided in FIG. 80.

The results of the mouse embryonic fibroblast p65, p50, and RelB knock-outs for AD037 are provided in FIG. 81.

### Example 10

### Method of Assessing the Effect of the NFkB Associated Sequences of the Present Invention on IL-8 Expression

H292 cells were plated in 48 well plates (65,000 cells/well) and cultured overnight. The cells were transfected using Lipofectamine 2000 (Invitrogen) according to manufacturer's instructions. Cells were transfected with either pcDNA vector containing a FLAG epitope tag, or pcDNA-FLAG encoding wild type IKK2, wild type AD037, AD037 with a deletion in a putative myristoylation site (AA26-31), or AD037 with a deletion of the ras association domain (AA167-263). The mutants were generated using the Transformer Site-Directed Mutagenesis Kit according to manufacturer's instructions (Clontech). Cells were cultured overnight with DNA complexes. The following day, the media was replaced with RPMI containing 0.25% FCS. The cells were stimulated with and without 1 ng/ml TNFα for 6 hours. Supernatants were assayed for IL-8 by ELISA. The cells were cultured for an additional 2 hours with MTS reagent (Promega) to monitor cell number. The IL-8 values are corrected for cell number using the MTS assay results.

Cos7 cells were transfected using Lipofectamine PLUS reagent (Invitrogen) with 5 ug of either vector, wild type AD037, or the two mutants described above. After resting for 24 hours, the cells were harvested using trypsin, washed with PBS, and either lysed in RIPA buffer (10 mM sodium phosphate pH 7.2, 0.25 M sodium chloride, 0.1% SDS, 1% NP40, 1% sodium deoxycholate, 2 mM EDTA, protease inhibitor cocktail) for Western blot analysis.

Cos7 cells were transfected using Lipofectamine PLUS reagent (Invitrogen) with 5 ug of either vector, wild type AD037, or the two mutants described above. After resting for 24 hours, the cells were harvested using trypsin, washed with PBS, and either lysed in RIPA buffer (10 mM sodium phosphate pH 7.2, 0.25 M sodium chloride, 0.1% SDS, 1% NP40, 1% sodium deoxycholate, 2 mM EDTA, protease inhibitor cocktail) for Western blot analysis.

For Western blotting, whole cell lysates were electrophoresed through 4-20% Tris-glycine gels (Novex, San Diego, Calif.), transferred to nitrocellulose, and blocked overnight in 5% BSA in Tris buffered saline. Blots were probed with a mouse monoclonal IgG specific for the Flag epitope tag (Sigma, St. Louis, Mo.), followed by detection with HRP-conjugated antibodies specific for mouse IgG, and ECL (Amersham Pharmacia Biotech, Piscataway, N.J.).

In the case of AD037, the results of AD037 expression on IL-8 expression are provided in FIG. 82.

In the case of AD037, the results of Western blotting for the expression of the wild type AD037, the ras deletion AD037 mutant, and the myristoylation site deletion AD037 mutant are provided in FIG. 83.

In the case of AD037, the results of expression of the wild type AD037, the ras deletion AD037 mutant, and the myristoylation site deletion AD037 mutant on IL-8 expression are provided in FIG. 84.

### Example 11

### Method of Assessing the Expression Profile of the NFkB Associated Sequences of the Present Invention Using Northern Blots

Other methods of assessing the expression profile of the NFkB associated sequences of the present invention are known in the art or otherwise referenced herein. For example. The tissue distribution of mRNA expression of polynucleotides of the present invention is determined using protocols for Northern blot analysis, described by, among others, Sambrook et al. For example, a cDNA probe produced by the method described in Example 2 is labeled with p32 using the rediprime(tm) DNA labeling system (Amersham Life Science), according to manufacturer's instructions. After labeling, the probe is purified using CHROMA SPINO-100 column (Clontech Laboratories, Inc.) according to manufacturer's protocol number PT1200-1. The purified labeled probe is then used to examine various tissues for mRNA expression.

Tissue Northern blots containing the bound mRNA of various tissues are examined with the labeled probe using ExpressHybtm hybridization solution (Clonetech according to manufacturers protocol number PT1190-1. Northern blots can be produced using various protocols well known in the art (e.g., Sambrook et al). Following hybridization and washing, the blots are mounted and exposed to film at −70° C. overnight, and the films developed according to standard procedures.

### Example 12

### Method of Confirming the Functional Relevance of the Polynucleotides and Polypeptides of the Present Invention to the NFkB Pathway through the Application of Antisense Oligonucleotide Methodology

Antisense oligonucleotides specific for each sequence may be synthesized. The oligonucleotides may be clectroporated into THP-1 cells. The cells may be cultured at 37°, 5% CO2 in RPMI 1640 (Life Technologies) supplemented with 2 mM L-glutamine, and 10% fetal calf serum at a density of 10⁶/ml for 24 hours following the electroporation. The cells may be collected by centrifugation and cultured for 6 hours with 100 ng/ml LPS (S. typhosa 0901, Sigma) at a density of 10⁶/ml in each well of a 96 well plate. At the end of the incubation, the plates are centrifuged, and supernatants are assayed for TNFα levels using an ELISA kit (Pharmingen).

Alternatively, another antisense olignucleotide assay for confirming the association of any one or more of the NFkB associated polynucleotides and polypeptide of the present invention to modulation of or modulation by NFkB , or the NFkB pathway, in general, may be applied. The assay is described below, in brief.

### Day 0

Plates are coated with Collagen. For one plate, Collagen is stored at 4° at 0.4 mg/ml until needed. 112.5 ul of glacial acetic acid is added to 13.5 ml of H2O, and then 84.35 ul of collagen is added to 13.5 ml of acetic acid. 250 ul is addedto each well and incubated for 2 hr at room temperature (final concentration is 2.5 ug/ml). Collagen is removed amd rinsed with 500 ul of PBS 2×. 200 ul of media is added and kept at 37° until read for use. HMVEC cells are then plated at 30 k/well in 48 well plates.

### Day 1

HMVEC cells are transfected using lug/ml Lipofectamine 2000 lipid and 25 nM antisense oligonucleotide according to the following protocol.

### Materials needed

HMVEC cells maintained in EBM-2 (Clonetics) supplemented with EGM-2 MV (Clonetics).

Opti-MEM (Gibco-BRL)

Lipofectamine 2000 (Invitrogen)

Antisense oligomers (Sequitur)

Polystyrene tubes

Tissue culture treated plates

A 10× stock of Lipofectamine 2000 (10 ug/ml is 10×) is prepared, and the diluted lipid is allowed to stand at RT for 15 minutes. Stock solution of Lipofectamine 2000 is 1 mg/ml. 10× solution for transfection is 10 ug/ml. To prepare 10× solution, dilute 10 ul of Lipofectamine 2000 stock per 1 ml of Opti-MEM (serum free media).

A 10× stock of each oligomer to be used in the transfection is then prepared. Stock solutions of oligomers are at 100 uM in 20 mM HEPES, pH 7.5. 10× concentration of oligomer is 0.25 uM. To prepare the 10× solutions, dilute 2.5 ul of oligomer per 1 ml of Opti-MEM.

Equal volumes of the 10× Lipofectamine 2000 stock and the 10× oligomer solutions. Mix well and incubate for 15 minutes at RT to allow complexation of the oligomer and lipid. The resulting mixture is 5×. After the 15 minute complexation, 4 volumes of full growth media is added to the oligomer/lipid complexes (solution is now 1×). The media is then aspirated from the cells, and 0.5 ml of the 1× oligomer/lipid complexes is added to each well.

The cells are incubated for 16-24 hours at 37° C. in a humidified CO₂ incubator. Oligomer update is evaluated by fluorescent microscopy. In addition, the cell viability is evaluated by performing dead stain analysis

### Day 2: Begin TNF Stimulation

TNF stored in −70° bottom shelf in 10 ul aliquots at concentration of 50 ug/ml. Two fold dilutions of TNF are made by first adding 10 ul to 1 mI to give 500 ng/ml of the TNF aliquots. Then 300 ul is added to 15 ml to give 10 ng/ml. 250 ul of this final solution is added to each well, and the cells are stimulated for 6 hours at 37°.

After stimulation, 100 ul of supernatant is removed from each well and stored at −70°. The remaining media is then removed from each well.

The cells are then titered. 200 ul of fresh media is added to each well. 50 ul CTR (cell titer reagent) is added to each well. Two blank wells are included for controls with just media and CTR. The cells are Incubated at 37° for about 90 minutes. 100 ul is removed from each well and moved to a 96 well plate. The absorbance is then read at 490 nm on spectrophotometer.

During the 90 minute incubation, a glutaraldehyde solution is prepared. 140 ul glutaraldehyde is added to 14 ml PBS (0.5% glutaraldehyde). Blocking buffer is also prepared. For one plate, make 50 ml: add 46.5 ml PBS, 1.5 ml goat serum (aliquots in −20° freezer) and 2 ml 0.5M EDTA.

Once cell titer is done, the remaining media is removed and 250 ul glutaraldehyde solution is added to each well, and incubated for 10 minutes at 4°. The plates are then flicked, and 500 ul blocking buffer is added to each well. The plates are then Incubated at 4° overnight.

### Day 3: Prepare E-selectin Solution

22.5 ul of 100 ug/ml stock is added to 9 ml blocking buffer. 150 ul is added to each well, and incubated for 1 hour at 37°. The wells are washed 4× with cold PBS, the plates are flicked between washes and then aspirated at the end to remove remaining PBS.

Prep HRP by adding 2.25 ul HRP (stored at 4°; top shelf) to 9 ml blocking buffer. 150 ul is added to each well, and incubated for 1 hour at 37°. The wells are washed 4× with cold PBS, and plates are flicked between washes and then aspirated at the end to remove remaining PBS. 150 ul peroxidase color reagent is added to each well for development. The plates are allowed to develop for about 5 minutes and stoped with 150 ul in H2SO4. 100 ul/wellis then transferred from each well to a 96 well plate, and the OD read at 450 nm.

The positives are then noted. It is expected that at least one or more of the NFkB associated polynucleotides and polypeptides of the present invention show a positive result in this assay. Any positives would provide convincing evidence that the sequences are involved in the NFkB pathway, either directly or indirectly. Specifically, AP002338, 30507, and AC010791 were all shown to result in inhibition of E-selectin expression in HMVEC cells in the above assay.

### Example 13

### Additional Methods of Confirming the Functional Relevance of the Polynucleotides and Polypeptides of the Present Invention to the NFkB Pathway through the Application of Antisense Oligonucleotide Methodology

Jurkat T cells will be transfected with antisense oligonucleotides specific for the clones, the transfected cells will then be stimulated with antibodies specific to both CD3 and CD28; and the level of IL-2 secretion in the supernatant measured using methods well known in the art (e.g., ELISA, immunoprecipitation, etc.). Antisense reagents that inhibit IL-2 secretion would suggest that the corresponding polynucleotides of the present invention are involved in an NF-kB dependent response.

The antisense oligonucleotides will also be used to identify the polynucleotides of the present invention that are involved in a B cell NF-kB dependent response. The human Raji B cell line will be transfected with antisense oligonucleotides, and then stimulated with anti-CD40 antibodies to induce homotypic aggregation. Inhibition of aggregation by an antisense oligonucleotide would suggest that the corresponding polynucleotides of the present invention are involved in an NF-kB response.

Moreover, the selectivity of the inhibition of homotypic aggregation in THP-1 cells. The cells will be transfected with antisense oligonucleotides and stimulated with either LPS or IFN-γ overnight to induce ICAM-1 expression. Induction by IFN-γ is mediated by the transcription factor STAT-1. Induction by LPS is mediated by NF-kB. Antisense oligonucleotides that inhibit LPS-induced, but not IFN-γ induced ICAM-1 suggest that the corresponding polynucleotides of the present invention are involved in an NF-kB pathway.

Additional methods for characterizing the NFkB associated polynucleotide and polypeptides of the invention are provided in U.S. Pat. No. 6,150,090 which is hereby incorporated herein in its entirety.

### Example 14

### Method of Isolating the Full-Length Polynucleotide of a NFkB Associated Polynucleotide of the Present Invention

The polynucleotide(s) of the present invention, the polynucleotide encoding the polypeptide of the present invention may represent partial, or incomplete versions of the complete coding region (i.e., full-length gene). Several methods are known in the art for the identification of the 5′ or 3′ non-coding and/or coding portions of a gene which may not be present in a clone. The methods that follow are exemplary and should not be construed as limiting the scope of the invention. These methods include but are not limited to, filter probing, clone enrichment using specific probes, and protocols similar or identical to 5′ and 3′ “RACE” protocols that are well known in the art. For instance, a method similar to 5′ RACE is available for generating the missing 5′ end of a desired full-length transcript. (Fromont-Racine et al., Nucleic Acids Res. 21(7):1683-1684 (1993)).

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full-length gene.

This above method starts with total RNA isolated from the desired source, although poly-A+ RNA can be used. The RNA preparation can then be treated with phosphatase if necessary to eliminate 5′ phosphate groups on degraded or damaged RNA that may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene. Moreover, it may be advantageous to optimize the RACE protocol to increase the probability of isolating additional 5′ or 3′ coding or non-coding sequences. Various methods of optimizing a RACE protocol are known in the art, though a detailed description summarizing these methods can be found in B.C. Schaefer, Anal. Biochem., 227:255-273, (1995).

An alternative method for carrying out 5′ or 3′ RACE for the identification of coding or non-coding sequences is provided by Frohman, M.A., et al., Proc.Nat'l.Acad.Sci.USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation, therefor. The following briefly describes a modification of this original 5′ RACE procedure. Poly A+ or total RNAs reverse transcribed with Superscript II (Gibco/BRL) and an antisense or I complementary primer specific to the cDNA sequence. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoIJ Sail and ClaI) at the 5′ end and a primer containing just these restriction sites. This double-stranded cDNA is PCR amplified for 40 cycles with the same primers as well as a nested cDNA-specific antisense primer. The PCR products are size-separated on an ethidium bromide-agarose gel and the region of gel containing cDNA products the predicted size of missing protein-coding DNA is removed. cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length genes. A second kit is available from Clontech which is a modification of a related technique, SLIC (single-stranded ligation to single-stranded cDNA), developed by Dumas et al., Nucleic Acids Res., 19:5227-32(1991). The major differences in procedure are that the RNA is alkaline hydrolyzed after reverse transcription and RNA ligase is used to join a restriction site-containing anchor primer to the first-strand cDNA. This obviates the necessity for the dA-tailing reaction which results in a polyT stretch that is difficult to sequence past.

An alternative to generating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

### RNA Ligase Protocol For Generating The 5′ or 3′ End Sequences to Obtain full Length Genes

Once a gene of interest is identified, several methods are available for the identification of the 5′ or 3′ portions of the gene which may not be present in the original cDNA plasmid. These methods include, but are not limited to, filter probing, clone enrichment using specific probes and protocols similar and identical to 5′ and 3′RACE. While the full-length gene may be present in the library and can be identified by probing, a useful method for generating the 5′ or 3′ end is to use the existing sequence information from the original cDNA to generate the missing information. A method similar to 5′RACE is available for generating the missing 5′ end of a desired full-length gene. (This method was published by Fromont-Racine et al., Nucleic Acids Res., 21(7): 1683-1684 (1993)). Briefly, a specific RNA oligonucleotide is ligated to the 5′ ends of a population of RNA presumably 30 containing full-length gene RNA transcript and a primer set containing a primer specific to the ligated RNA oligonucleotide and a primer specific to a known sequence of the gene of interest, is used to PCR amplify the 5′ portion of the desired full length gene which may then be sequenced and used to generate the full length gene. This method starts with total RNA isolated from the desired source, poly A RNA may be used but is not a prerequisite for this procedure. The RNA preparation may then be treated with phosphatase if necessary to eliminate 5′ phosphate groups on degraded or damaged RNA which may interfere with the later RNA ligase step. The phosphatase if used is then inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a gene specific oligonucleotide. The first strand synthesis reaction can then be used as a template for PCR amplification of the desired 5′ end using a primer specific to the ligated RNA oligonucleotide and a primer specific to the known sequence of the apoptosis related of interest. The resultant product is then sequenced and analyzed to confirm that the 5′ end sequence belongs to the relevant apoptosis related.

### Example 15

### Chromosomal Mapping of the Polynucleotides

An oligonucleotide primer set is designed according to the sequence at the 5′ end of SEQ ID NO:1-108, 125, 127, 132-140, 158-159, or 264-284. This primer preferably spans about 100 nucleotides. This primer set is then used in a polymerase chain reaction under the following set of conditions: 30 seconds,95 degree C.; 1 minute, 56 degree C.; 1 minute, 70 degree C. This cycle is repeated 32 times followed by one 5 minute cycle at 70 degree C. Mammalian DNA, preferably human DNA, is used as template in addition to a somatic cell hybrid panel containing individual chromosomes or chromosome fragments (Bios, Inc). The reactions are analyzed on either 8% polyacrylamide gels or 3.5% agarose gels. Chromosome mapping is determined by the presence of an approximately 100 bp PCR fragment in the particular somatic cell hybrid.

### Example 16

### Bacterial Expression of A Polypeptide

A polynucleotide encoding a polypeptide of the present invention is amplified using PCR oligonucleotide primers corresponding to the 5′ and 3′ ends of the DNA sequence, as outlined in Example 14, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the laci repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni—NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni—NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 17

### Purification of A Polypeptide from an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 18

### Cloning and Expression of A Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in Example 14, to synthesize insertion fragments. The primers used to amplify the cDNA insert should preferably contain restriction sites at the 5′ end of the primers in order to clone the amplified product into the expression vector. Specifically, the cDNA sequence contained in a clone, including the AUG initiation codon and the naturally associated leader sequence identified elsewhere herein (if applicable), is amplified using the PCR protocol described in Example 14. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with I ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 19

### Expression of A Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### Example 20

### Method of Creating N- and C-Terminal Deletion Mutants Corresponding to the NFkB-Associated Polypeptides of the Present Invention

As described elsewhere herein, the present invention encompasses the creation of N- and C-terminal deletion mutants, in addition to any combination of N- and C-terminal deletions thereof, corresponding to the NFkB-associated polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length NFkB-associated polypeptide sequence (as described in Example 14, Table I, or Table III, for example), appropriate primers of about 15-25 nucleotides derived from the desired 5′ and 3′ positions of, for example, SEQ ID NO:125 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the P12 to K321 AD037 N-terminal deletion mutant, the following primers could be used to amplify a cDNA fragment corresponding to this deletion mutant:

5′ Primer

(SEQ ID NO:168)

5′-GCAGCA GCGGCCGC CCCATCAGTGACAGCAAGTCCATTC-3′

[symbol]

3′ Primer

(SEQ ID NO:169)

5′-GCAGCA GTCGAC CTTGGCCTCCACCAGCTGCTCCAGG-3′

[symbol]

For example, in the case of the M1 to K289 AD037 C-terminal deletion mutant, the following primers could be used to amplify a cDNA fragment corresponding to this deletion mutant:

5′ Primer

(SEQ ID NO:170)

5′-GCAGCA GCGGCCGC ATGAAGGAAGACTGTCTGCCGAG-3′

[symbol]

3′ Primer

(SEQ ID NO:171)

5′-GCAGCA GTCGAC TTTTAATTTTTCAACAAAACTGTCC-3′

[symbol]

Representative PCR amplification conditions are provided below, although the skilled artisan would appreciate that other conditions may be required for efficient amplification. A 100 ul PCR reaction mixture may be prepared using long of the template DNA (cDNA clone of a NFkB-associated clone), 200 uM 4dNTPs, 1 uM primers, 0.25 U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5 U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula: (S+(X * 3)) to ((S+(X * 3))+25), wherein ‘S’ is equal to the nucleotide position of the initiating start codon of a NFkB-associated gene (e.g., AD037; SEQ ID NO:125), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of, for example, SEQ ID NO:125. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula: (S+(X * 3)) to ((S+(X * 3))−25), wherein ‘S’ is equal to the nucleotide position of the initiating start codon of a NFkB-associated gene (e.g., AD037; SEQ ID NO:125), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of, for example, e.g., SEQ ID NO:125. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any N- or C-terminal deletion mutant of the present invention (e.g., corresponding to the polypeptides provided as SEQ ID NO:109-118, 126, 128, 144-152, 160, and 161). Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

Primer sequences required to create N- and/or C-terminal deletions of the other NFkB associated sequences of the present invention could be designed based upon the teachings of the present invention and the application of methods well known in the art of molecular biology.

### Example 21

### Protein Fusions

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example described herein; see also EP A 394,827; Traunecker, et al., Nature 331:84-86 (1988).) Similarly, fusion to IgG-1, IgG-3, and albumin increases the half-life time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector. Note that the polynucleotide is cloned without a stop codon, otherwise a fusion protein will not be produced.

The naturally occurring signal sequence may be used to produce the protein (if applicable). Alternatively, if the naturally occurring signal sequence is not used, the vector can be modified to include a heterologous signal sequence. (See, e.g., WO 96/34891 and/or U.S. Pat. No. 6,066,781, supra.)

### Human IgG Fc Region

(SEQ ID NO:123)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCAAGCGACATCGCCGTG
GAGTGGGAGAGCAATGGGCAGCCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAATGAGTGCGACGGCCGCGACTCTAGAGGAT

### Example 22

### Regulation of Protein Expression via Controlled Aggregation in the Endoplasmic Reticulum

As described more particularly herein, proteins regulate diverse cellular processes in higher organisms, ranging from rapid metabolic changes to growth and differentiation. Increased production of specific proteins could be used to prevent certain diseases and/or disease states. Thus, the ability to modulate the expression of specific proteins in an organism would provide significant benefits.

Numerous methods have been developed to date for introducing foreign genes, either under the control of an inducible, constitutively active, or endogenous promoter, into organisms. Of particular interest are the inducible promoters (see, M. Gossen, et al., Proc. Natl. Acad. Sci. USA., 89:5547 (1992); Y. Wang, et al., Proc. Natl. Acad. Sci. USA, 91:8180 (1994), D. No., et al., Proc. Natl. Acad. Sci. USA, 93:3346 (1996); and V. M. Rivera, et al., Nature Med, 2:1028 (1996); in addition to additional examples disclosed elsewhere herein). In one example, the gene for erthropoietin (Epo) was transferred into mice and primates under the control of a small molecule inducer for expression (e.g., tetracycline or rapamycin) (see, D. Bohl, et al., Blood, 92:1512, (1998); K. G. Rendahl, et al., Nat. Biotech, 16:757, (1998); V. M. Rivera, et al., Proc. Natl. Acad. Sci. USA, 96:8657 (1999); and X.Ye et al., Science, 283:88 (1999). Although such systems enable efficient induction of the gene of interest in the organism upon addition of the inducing agent (i.e., tetracycline, rapamycin, etc.), the levels of expression tend to peak at 24 hours and trail off to background levels after 4 to 14 days. Thus, controlled transient expression is virtually impossible using these systems, though such control would be desirable.

A new alternative method of controlling gene expression levels of a protein from a transgene (i.e., includes stable and transient transformants) has recently been elucidated (V. M. Rivera., et al., Science, 287:826-830, (2000)). This method does not control gene expression at the level of the mRNA like the aforementioned systems. Rather, the system controls the level of protein in an active secreted form. In the absence of the inducing agent, the protein aggregates in the ER and is not secreted. However, addition of the inducing agent results in dis-aggregation of the protein and the subsequent secretion from the ER. Such a system affords low basal secretion, rapid, high level secretion in the presence of the inducing agent, and rapid cessation of secretion upon removal of the inducing agent. In fact, protein secretion reached a maximum level within 30 minutes of induction, and a rapid cessation of secretion within 1 hour of removing the inducing agent. The method is also applicable for controlling the level of production for membrane proteins.

Detailed methods are presented in V. M. Rivera., et al., Science, 287:826-830, (2000)), briefly:

Fusion protein constructs are created using polynucleotide sequences of the present invention with one or more copies (preferably at least 2, 3, 4, or more) of a conditional aggregation domain (CAD) a domain that interacts with itself in a ligand- reversible manner (i.e., in the presence of an inducing agent) using molecular biology methods known in the art and discussed elsewhere herein. The CAD domain may be the mutant domain isolated from the human FKBP12 (Phe³⁶ to Met) protein (as disclosed in V. M. Rivera., et al., Science, 287:826-830, (2000), or alternatively other proteins having domains with similar ligand-reversible, self-aggregation properties. As a principle of design the fusion protein vector would contain a furin cleavage sequence operably linked between the polynucleotides of the present invention and the CAD domains. Such a cleavage site would enable the proteolytic cleavage of the CAD domains from the polypeptide of the present invention subsequent to secretion from the ER and upon entry into the trans-Golgi (J. B. Denault, et al., FEBS Lett., 379:113, (1996)). Alternatively, the skilled artisan would recognize that any proteolytic cleavage sequence could be substituted for the furin sequence provided the substituted sequence is cleavable either endogenously (e.g., the furin sequence) or exogenously (e.g., post secretion, post purification, post production, etc.). The preferred sequence of each feature of the fusion protein construct, from the 5′ to 3′ direction with each feature being operably linked to the other, would be a promoter, signal sequence, “X” number of (CAD)x domains, the furin sequence (or other proteolytic sequence), and the coding sequence of the polypeptide of the present invention. The artisan would appreciate that the promotor and signal sequence, independent from the other, could be either the endogenous promotor or signal sequence of a polypeptide of the present invention, or alternatively, could be a heterologous signal sequence and promotor.

The specific methods described herein for controlling protein secretion levels through controlled ER aggregation are not meant to be limiting are would be generally applicable to any of the polynucleotides and polypeptides of the present invention, including variants, homologues, orthologs, and fragments therein.

### Example 23

### Alteration of Protein Glycosylation Sites to Enhance Characteristics of Polypeptides of the Invention

Many eukaryotic cell surface and proteins are post-translationally processed to incorporate N-linked and O-linked carbohydrates (Kornfeld and Kornfeld (1985) Annu. Rev. Biochem. 54:631-64; Rademacher et al., (1988) Annu. Rev. Biochem. 57:785-838). Protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion (Fieldler and Simons (1995) Cell, 81:309-312; Helenius (1994) Mol. Biol. Of the Cell 5:253-265; Olden et al., (1978) Cell, 13:461-473; Caton et al., (1982) Cell, 37:417-427; Alexamnder and Elder (1984), Science, 226:1328-1330; and Flack et al., (1994), J. Biol. Chem., 269:14015-14020). In higher organisms, the nature and extent of glycosylation can markedly affect the circulating half-life and bio-availability of proteins by mechanisms involving receptor mediated uptake and clearance (Ashwell and Morrell, (1974), Adv. Enzymol., 41:99-128; Ashwell and Harford (1982), Ann. Rev. Biochem., 51:531-54). Receptor systems have been identified that are thought to play a major role in the clearance of serum proteins through recognition of various carbohydrate structures on the glycoproteins (Stockert (1995), Physiol. Rev., 75:591-609; Kery et al., (1992), Arch. Biochem. Biophys., 298:49-55). Thus, production strategies resulting in incomplete attachment of terminal sialic acid residues might provide a means of shortening the bioavailability and half-life of glycoproteins. Conversely, expression strategies resulting in saturation of terminal sialic acid attachment sites might lengthen protein bioavailability and half-life.

In the development of recombinant glycoproteins for use as pharmaceutical products, for example, it has been speculated that the pharmacodynamics of recombinant proteins can be modulated by the addition or deletion of glycosylation sites from a glycoproteins primary structure (Berman and Lasky (1985a) Trends in Biotechnol., 3:51-53). However, studies have reported that the deletion of N-linked glycosylation sites often impairs intracellular transport and results in the intracellular accumulation of glycosylation site variants (Machamer and Rose (1988), J. Biol Chem., 263:5955-5960; Gallagher et al., (1992), J. Virology., 66:7136-7145; Collier et al., (1993), Biochem., 32:7818-7823; Claffey et al., (1995) Biochemica et Biophysica Acta, 1246:1-9; Dube et al., (1988), J. Biol. Chem. 263:17516-17521). While glycosylation site variants of proteins can be expressed intracellularly, it has proved difficult to recover useful quantities from growth conditioned cell culture medium.

Moreover, it is unclear to what extent a glycosylation site in one species will be recognized by another species glycosylation machinery. Due to the importance of glycosylation in protein metabolism, particularly the secretion and/or expression of the protein, whether a glycosylation signal is recognized may profoundly determine a proteins ability to be expressed, either endogenously or recombinately, in another organism (i.e., expressing a human protein in E.coli, yeast, or viral organisms; or an E.coli, yeast, or viral protein in human, etc.). Thus, it may be desirable to add, delete, or modify a glycosylation site, and possibly add a glycosylation site of one species to a protein of another species to improve the proteins functional, bioprocess purification, and/or structural characteristics (e.g., a polypeptide of the present invention).

A number of methods may be employed to identify the location of glycosylation sites within a protein. One preferred method is to run the translated protein sequence through the PROSITE computer program (Swiss Institute of Bioinformatics). Once identified, the sites could be systematically deleted, or impaired, at the level of the DNA using mutagenesis methodology known in the art and available to the skilled artisan, Preferably using PCR-directed mutagenesis (See Maniatis, Molecular Cloning: A Laboratory Manual, Cold Spring Harbor Press, Cold Spring, N.Y. (1982)). Similarly, glycosylation sites could be added, or modified at the level of the DNA using similar methods, preferably PCR methods (See, Maniatis, supra). The results of modifying the glycosylation sites for a particular protein (e.g., solubility, secretion potential, activity, aggregation, proteolytic resistance, etc.) could then be analyzed using methods know in the art.

The skilled artisan would acknowledge the existence of other computer algorithms capable of predicting the location of glycosylation sites within a protein. For example, the Motif computer program (Genetics Computer Group suite of programs) provides this function, as well.

### Example 24

### Method of Enhancing the Biological Activity/Functional Characteristics of Invention through Molecular Evolution

Although many of the most biologically active proteins known are highly effective for their specified function in an organism, they often possess characteristics that make them undesirable for transgenic, therapeutic, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered NFkB associated protein may be constitutively active upon binding of its cognate ligand. Alternatively, an engineered NFkB associated protein may be constitutively active in the absence of ligand binding. In yet another example, an engineered NFkB associated protein may be capable of being activated with less than all of the regulatory factors and/or conditions typically required for NFkB associated protein activation (e.g., ligand binding, phosphorylation, conformational changes, etc.). Such NFkB associated protein would be useful in screens to identify NFkB modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, DE, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments—further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatmann) or could be purified using Microcon concentrators (Amicon) of the appropriate molecular weight cutoff, or could use oligonucleotide purification columns (Qiagen), in addition to other methods known in the art. If using DE81 ion-exchange paper, the 10-50 bp fragments could be eluted from said paper using 1M NaCl, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris·HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C. for 60s; 94 C. for 30s, 50-55 C. for 30 s, and 72 C. for 30 s using 30-45 cycles, followed by 72 C. for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C. for 30 s, 50 C. for 30 s, and 72 C. for 30 s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self′ molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 25

### Method of Determining Alterations in a Gene Corresponding to A Polynucleotide

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1-108, 125, 127, 132-140, 158-159, or 264-284. Suggested PCR conditions consist of 35 cycles at 95 degrees C. for 30 seconds; 60-120 seconds at 52-58 degrees C.; and 60-120 seconds at 70 degrees C., using buffer solutions described in Sidransky et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products is cloned into T-tailed vectors as described in Holton et al., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 14 are nick-translated with digoxigenindeoxy-uridine 5′-triphosphate (Boehringer Manheim), and FISH performed as described in Johnson et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 26

### Method of Detecting Abnormal Levels of A Polypeptide in A Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/ml. The antibodies are either monoclonal or polyclonal and are produced by the method described elsewhere herein. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 27

### Formulation

The invention also provides methods of treatment and/or prevention diseases, disorders, and/or conditions (such as, for example, any one or more of the diseases or disorders disclosed herein) by administration to a subject of an effective amount of a Therapeutic. By therapeutic is meant a polynucleotides or polypeptides of the invention (including fragments and variants), agonists or antagonists thereof, and/or antibodies thereto, in combination with a pharmaceutically acceptable carrier type (e.g., a sterile carrier).

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Therapeutics can be administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

In yet an additional embodiment, the Therapeutics of the invention are delivered orally using the drug delivery technology described in U.S. Pat. No. 6,258,789, which is hereby incorporated by reference herein.

Therapeutics of the invention are also suitably administered by sustained-release systems. Suitable examples of sustained-release Therapeutics are administered orally, rectally, parenterally, intracisternally, intravaginally, intraperitoneally, topically (as by powders, ointments, gels, drops or transdermal patch), bucally, ′or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention may also be suitably administered by sustained-release systems. Suitable examples of sustained-release Therapeutics include suitable polymeric materials (such as, for example, semi-permeable polymer matrices in the form of shaped articles, e.g., films, or microcapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

Sustained-release Therapeutics also include liposomally entrapped Therapeutics of the invention (see, generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing the Therapeutic are prepared by methods known per se: DE 3,218,121; Epstein et al., Proc. Natl. Acad. Sci. (USA) 82:3688-3692 (1985); Hwang et al., Proc. Natl. Acad. Sci.(USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The Therapeutic will typically be formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

The Therapeutics of the invention may be administered alone or in combination with adjuvants. Adjuvants that may be administered with the Therapeutics of the invention include, but are not limited to, alum, alum plus deoxycholate (ImmunoAg), MTP-PE (Biocine Corp.), QS21 (Genentech, Inc.), BCG, and MPL. In a specific embodiment, Therapeutics of the invention are administered in combination with alum. In another specific embodiment, Therapeutics of the invention are administered in combination with QS-21. Further adjuvants that may be administered with the Therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the Therapeutics of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The Therapeutics of the invention may be administered alone or in combination with other therapeutic agents. Therapeutic agents that may be administered in combination with the Therapeutics of the invention, include but not limited to, other members of the TNF family, chemotherapeutic agents, antibiotics, steroidal and non-steroidal anti-inflammatories, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the Therapeutics of the invention are administered in combination with members of the TNF family. TNF, TNF-related or TNF-like molecules that may be administered with the Therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-IBBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-EBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892),TR10 (International Publication No. WO 98/54202), 312C2 (International Publication No. WO 98/06842), and TR12, and soluble forms CD154, CD70, and CD153.

In certain embodiments, Therapeutics of the invention are administered in combination with antiretroviral agents, nucleoside reverse transcriptase inhibitors, non-nucleoside reverse transcriptase inhibitors, and/or protease inhibitors. Nucleoside reverse transcriptase inhibitors that may be administered in combination with the Therapeutics of the invention, include, but are not limited to, RETROVIR (zidovudine/AZT), VIDEX (didanosine/ddl), HIVID (zalcitabine/ddC), ZERIT (stavudine/d4T), EPIVIR (lamivudine/3TC), and COMBIVIR (zidovudine/lamivudine). Non-nucleoside reverse transcriptase inhibitors that may be administered in combination with the Therapeutics of the invention, include, but are not limited to, VIRAMUNE (nevirapine), RESCRIPTOR (delavirdine), and SUSTIVA (efavirenz). Protease inhibitors that may be administered in combination with the Therapeutics of the invention, include, but are not limited to, CRIXIVAN (indinavir), NORVIR (ritonavir), INVIRASE (saquinavir), and VIRACEPT (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In other embodiments, Therapeutics of the invention may be administered in combination with anti-opportunistic infection agents. Anti-opportunistic agents that may be administered in combination with the Therapeutics of the invention, include, but are not limited to, TRIMETHOPRIM-SULFAMETHOXAZOLE, DAPSONE, PENTAMIDINE, ATOVAQUONE, ISONIAZID, RIFAMPIN, PYRAZINAMIDE, ETHAMBUTOL, RIFABUTIN, CLARITHROMYCIN, AZITHROMYCIN, GANCICLOVIR, FOSCARNET, CIDofOVIR, FLUCONAZOLE, ITRACONAZOLE, KETOCONAZOLE, ACYCLOVIR, FAMCICOLVIR, PYRIMETHAMINE, LEUCOVORIN, NEUPOGEN (filgrastim/G-CSF), and LEUKINE (sargramostim/GM-CSF). In a specific embodiment, Therapeutics of the invention are used in any combination with TRIMETHOPRIM-SULFAMETHOXAZOLE, DAPSONE, PENTAMIDINE, and/or ATOVAQUONE to prophylactically treat or prevent an opportunistic Pneumocystis carinii pneumonia infection. In another specific embodiment, Therapeutics of the invention are used in any combination with ISONIAZID, RIFAMPIN, PYRAZINAMIDE, and/or ETHAMBUTOL to prophylactically treat or prevent an opportunistic Mycobacterium avium complex infection. In another specific embodiment, Therapeutics of the invention are used in any combination with RIFABUTIN, CLARITHROMYCIN, and/or AZITHROMYCIN to prophylactically treat or prevent an opportunistic Mycobacterium tuberculosis infection. In another specific embodiment, Therapeutics of the invention are used in any combination with GANCICLOVIR, FOSCARNET, and/or CIDOFOVIR to prophylactically treat or prevent an opportunistic cytomegalovirus infection. In another specific embodiment, Therapeutics of the invention are used in any combination with FLUCONAZOLE, ITRACONAZOLE, and/or KETOCONAZOLE to prophylactically treat or prevent an opportunistic fungal infection. In another specific embodiment, Therapeutics of the invention are used in any combination with ACYCLOVIR and/or FAMCICOLVIR to prophylactically treat or prevent an opportunistic herpes simplex virus type I and/or type II infection. In another specific embodiment, Therapeutics of the invention are used in any combination with PYRIMETHAMINE and/or LEUCOVORIN to prophylactically treat or prevent an opportunistic Toxoplasma gondii infection. In another specific embodiment, Therapeutics of the invention are used in any combination with LEUCOVORIN and/or NEUPOGEN to prophylactically treat or prevent an opportunistic bacterial infection.

In a further embodiment, the Therapeutics of the invention are administered in combination with an antiviral agent. Antiviral agents that may be administered with the Therapeutics of the invention include, but are not limited to, acyclovir, ribavirin, amantadine, and remantidine.

In a further embodiment, the Therapeutics of the invention are administered in combination with an antibiotic agent. Antibiotic agents that may be administered with the Therapeutics of the invention include, but are not limited to, amoxicillin, beta-lactamases, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

Conventional nonspecific immunosuppressive agents, that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, Therapeutics of the invention are administered in combination with immunosuppressants. Immunosuppressants preparations that may be administered with the Therapeutics of the invention include, but are not limited to, ORTHOCLONE (OKT3), SANDIMMUNE/NEORAL/SANGDYA (cyclosporin), PROGRAF (tacrolimus), CELLCEPT (mycophenolate), Azathioprine, glucorticosteroids, and RAPAMUNE (sirolimus). In a specific embodiment, immunosuppressants may be used to prevent rejection of organ or bone marrow transplantation.

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR, IVEEGAM, SANDOGLOBULIN, GAMMAGARD S/D, and GAMIMUNE. In a specific embodiment, Therapeutics of the invention are administered in combination with intravenous immune globulin preparations in transplantation therapy (e.g., bone marrow transplant).

In an additional embodiment, the Therapeutics of the invention are administered alone or in combination with an anti-inflammatory agent. Anti-inflammatory agents that may be administered with the Therapeutics of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the Therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, Therapeutics of the invention are administered in combination with CHOP (cyclophosphamide, doxorubicin, vincristine, and prednisone) or any combination of the components of CHOP. In another embodiment, Therapeutics of the invention are administered in combination with Rituximab. In a further embodiment, Therapeutics of the invention are administered with Rituxmab and CHOP, or Rituxmab and any combination of the components of CHOP.

In an additional embodiment, the Therapeutics of the invention are administered in combination with cytokines. Cytokines that may be administered with the Therapeutics of the invention include, but are not limited to, IL2, IL3, IL, IL5, IL6, IL7, IL10, IL12, IL13, IL15, anti-CD40, CD40L, IFN-gamma and TNF-alpha. In another embodiment, Therapeutics of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21.

In an additional embodiment, the Therapeutics of the invention are administered in combination with other immune factors. Immune factors that may be administered with the Therapeutics of the invention include, but are not limited to, Ly9, CD2, CD48, CD58, 2B4, CD84, CDw15O, CTLA4, CTLA4Ig, Bsl1, Bsl2, Bsl3, BLYS, TRAIL, APRIL, B7, B7 antagonists, B7 agonists, Ret16, APEX1, APEX2, APEX3, and APEX4.

In an additional embodiment, the Therapeutics of the invention are administered in combination with angiogenic proteins. Angiogenic proteins that may be administered with the Therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-6821 10; Platelet Derived Growth Factor-B (PDGF-B), as disclosed in European Patent Number EP-282317; Placental Growth Factor (PIGF), as disclosed in International Publication Number WO 92/06194; Placental Growth Factor-2 (PIGF-2), as disclosed in Hauser et al., Gorwth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the Therapeutics of the invention are administered in combination with hematopoietic growth factors. Hematopoietic growth factors that may be administered with the Therapeutics of the invention include, but are not limited to, LEUKINE (SARGRAMOSTIM) and NEUPOGEN (FILGRASTIM).

In an additional embodiment, the Therapeutics of the invention are administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In a specific embodiment, formulations of the present invention may further comprise antagonists of P-glycoprotein (also referred to as the multiresistance protein, or PGP), including antagonists of its encoding polynucleotides (e.g., antisense oligonucleotides, ribozymes, zinc-finger proteins, etc.). P-glycoprotein is well known for decreasing the efficacy of various drug administrations due to its ability to export intracellular levels of absorbed drug to the cell exterior. While this activity has been particularly pronounced in cancer cells in response to the administration of chemotherapy regimens, a variety of other cell types and the administration of other drug classes have been noted (e.g., T-cells and anti-HIV drugs). In fact, certain mutations in the PGP gene significantly reduces PGP function, making it less able to force drugs out of cells. People who have two versions of the mutated gene—one inherited from each parent—have more than four times less PGP than those with two normal versions of the gene. People may also have one normal gene and one mutated one. Certain ethnic populations have increased incidence of such PGP mutations. Among individuals from Ghana, Kenya, the Sudan, as well as African Americans, frequency of the normal gene ranged from 73% to 84%. In contrast, the frequency was 34% to 59% among British whites, Portuguese, Southwest Asian, Chinese, Filipino and Saudi populations. As a result, certain ethnic populations may require increased administration of PGP antagonist in the formulation of the present invention to arrive at the an efficacious dose of the therapeutic (e.g., those from African descent). Conversely, certain ethnic populations, particularly those having increased frequency of the mutated PGP (e.g., of Caucasian descent, or non-African descent) may require less pharmaceutical compositions in the formulation due to an effective increase in efficacy of such compositions as a result of the increased effective absorption (e.g., less PGP activity) of said composition.

Moreover, in another specific embodiment, formulations of the present invention may further comprise antagonists of OATP2 (also referred to as the multiresistance protein, or MRP2), including antagonists of its encoding polynucleotides (e.g., antisense oligonucleotides, ribozymes, zinc-finger proteins, etc.). The invention also further comprises any additional antagonists known to inhibit proteins thought to be attributable to a multidrug resistant phenotype in proliferating cells.

Preferred antagonists that formulations of the present may comprise include the potent P-glycoprotein inhibitor elacridar, and/or LY-335979. Other P-glycoprotein inhibitors known in the art are also encompassed by the present invention.

In additional embodiments, the Therapeutics of the invention are administered in combination with other therapeutic or prophylactic regimens, such as, for example, radiation therapy.

### Example 28

### Method of Treating Decreased Levels of the Polypeptide

The present invention relates to a method for treating an individual in need of an increased level of a polypeptide of the invention in the body comprising administering to such an individual a composition comprising a therapeutically effective amount of an agonist of the invention (including polypeptides of the invention). Moreover, it will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a Therapeutic comprising an amount of the polypeptide to increase the activity level of the polypeptide in such an individual.

For example, a patient with decreased levels of a polypeptide receives a daily dose 0.1-100 ug/kg of the polypeptide for six consecutive days. Preferably, the polypeptide is in the secreted form. The exact details of the dosing scheme, based on administration and formulation, are provided herein.

### Example 29

### Method of Treating Increased Levels of the Polypeptide

The present invention also relates to a method of treating an individual in need of a decreased level of a polypeptide of the invention in the body comprising administering to such an individual a composition comprising a therapeutically effective amount of an antagonist of the invention (including polypeptides and antibodies of the invention).

In one example, antisense technology is used to inhibit production of a polypeptide of the present invention. This technology is one example of a method of decreasing levels of a polypeptide, preferably a secreted form, due to a variety of etiologies, such as cancer. For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided herein.

### Example 30

### Method of Treatment Using Gene Therapy-ex vivo

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 14 using primers and having appropriate restriction sites and initiation/stop codons, if necessary. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 31

### Gene Therapy using Endogenous Genes Corresponding to Polynucleotides of the Invention

Another method of gene therapy according to the present invention involves operably associating the endogenous polynucleotide sequence of the invention with a promoter via homologous recombination as described, for example, in U.S. Pat. No.: 5,641,670, issued Jun. 24, 1997; International Publication NO:WO 96/29411, published Sep. 26, 1996; International Publication NO:WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous polynucleotide sequence, flanking the promoter. The targeting sequence will be sufficiently near the 5′ end of the polynucleotide sequence so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous polynucleotide sequence. This results in the expression of polynucleotide corresponding to the polynucleotide in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×106 cells/ml. Electroporation should be performed immediately following resuspension.

Plasmid DNA is prepared according to standard techniques. For example, to construct a plasmid for targeting to the locus corresponding to the polynucleotide of the invention, plasmid pUC18 (MBI Fermentas, Amherst, N.Y.) is digested with HindIII. The CMV promoter is amplified by PCR with an XbaI site on the 5′ end and a BamHI site on the 3′end. Two non-coding sequences are amplified via PCR: one non-coding sequence (fragment 1) is amplified with a HindIII site at the 5′ end and an Xba site at the 3′end; the other non-coding sequence (fragment 2) is amplified with a BamHI site at the 5′end and a HindIII site at the 3′end. The CMV promoter and the fragments (1 and 2) are digested with the appropriate enzymes (CMV promoter—XbaI and BamHI; fragment 1—XbaI; fragment 2—BamHI) and ligated together. The resulting ligation product is digested with HindIII, and ligated with the HindIII-digested pUC 18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5.×106 cells) is then added to the cuvette, and the cell suspension and DNA solutions are gently mixed. Electroporation is performed with a Gene-Pulser apparatus (Bio-Rad). Capacitance and voltage are set at 960 VF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 degree C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 32

### Method of Treatment Using Gene therapy—in vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. No. 5693622, 5705151, 5580859; Tabata et al., Cardiovasc. Res. 35(3):470-479 (1997); Chao et al., Pharmacol. Res. 35(6):517-522 (1997); Wolff, Neuromuscul. Disord. 7(5):314-318 (1997); Schwartz et al., Gene Ther. 3(5):405-411 (1996); Tsurumi et al., Circulation 94(12):3281-3290 (1996) (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 33

### Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR(RT-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 34

### Knock-Out Animals

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 35

### Method of Isolating Antibody Fragments Directed Against NF-kB-Associated Polypeptides from a Library of scFvs

Naturally occurring V-genes isolated from human PBLs are constructed into a library of antibody fragments which contain reactivities against NF-kB-associated polypeptides to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to inoculate 50 ml of 2×TY-AMP-GLU, 2×108 TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 pg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/mI (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 36

### Identification and Cloning of VH and VL Domains of Antibodies Directed Against the NF-kB-Associated Polypeptides Polypeptide

VH and VL domains may be identified and cloned from cell lines expressing an antibody directed against a NF-kB-associated polypeptides epitope by performing PCR with VH and VL specific primers on cDNA made from the antibody expressing cell lines. Briefly, RNA is isolated from the cell lines and used as a template for RT-PCR designed to amplify the VH and VL domains of the antibodies expressed by the EBV cell lines. Cells may be lysed using the TRIzol reagent (Life Technologies, Rockville, Md.) and extracted with one fifth volume of chloroform. After addition of chloroform, the solution is allowed to incubate at room temperature for 10 minutes, and then centrifuged at 14, 000 rpm for 15 minutes at 4 C. in a tabletop centrifuge. The supernatant is collected and RNA is precipitated using an equal volume of isopropanol. Precipitated RNA is pelleted by centrifuging at 14, 000 rpm for 15 minutes at 4 C. in a tabletop centrifuge.

Following centrifugation, the supernatant is discarded and washed with 75% ethanol. Follwing the wash step, the RNA is centrifuged again at 800 rpm for 5 minutes at 4 C. The supernatant is discarded and the pellet allowed to air dry. RNA is the dissolved in DEPC water and heated to 60 C. for 10 minutes. Quantities of RNA can be determined using optical density measurements. CDNA may be synthesized, according to methods well-known in the art and/or described herein, from 1. 5-2. 5 micrograms of RNA using reverse transciptase and random hexamer primers. CDNA is then used as a template for PCR amplification of VH and VL domains.

Primers used to amplify VH and VL genes are shown below. Typically a PCR reaction makes use of a single 5′ primer and a single 3′ primer. Sometimes, when the amount of available RNA template is limiting, or for greater efficiency, groups of 5′ and/or 3′ primers may be used. For example, sometimes all five VH-5′ primers and all JH3′ primers are used in a single PCR reaction. The PCR reaction is carried out in a 50 microliter volume containing 1×PCR buffer, 2 mM of each dNTP, 0. 7 units of High Fidelity Taq polymerse, 5′ primer mix, 3′ primer mix and 7. 5 microliters of cDNA. The 5′ and 3′ primer mix of both VH and VL can be made by pooling together 22 pmole and 28 pmole, respectively, of each of the individual primers. PCR conditions are: 96 C. for 5 minutes ; followed by 25 cycles of 94 C. for 1 minute, 50 C. for 1 minute, and 72 C. for 1 minute; followed by an extension cycle of 72 C. for 10 minutes. After the reaction has been completed, sample tubes may be stored at 4 C.

| | | |
|---|---|---|
| | | SEQ ID |
| Primer name | Primer Sequence | NO: |

Primer Sequences Used to Amplify VH domains

| | | |
|---|---|---|
| Hu VH1-5′ | CAGGTGCAGCTGGTGCAGTCTGG | 170 |
| Hu VH2-5′ | CAGGTCAACTTAAGGGAGTCTGG | 171 |
| Hu VH3-5′ | GAGGTGCAGCTGGTGGAGTCTGG | 172 |
| Hu VH4-5′ | CAGGTGCAGCTGCAGGAGTCGGG | 173 |
| Hu VH5-5′ | GAGGTGCAGCTGTTGCAGTCTGC | 174 |
| Hu VH6-5′ | CAGGTACAGCTGCAGCAGTCAGG | 175 |
| Hu JH1-5′ | TGAGGAGACGGTGACCAGGGTGCC | 176 |
| Hu JH3-5′ | TGAAGAGACGGTGACCATTGTCCC | 177 |
| Hu JH4-5′ | TGAGGAGACGGTGACCAGGGTTCC | 178 |
| Hu JH6-5′ | TGAGGAGACGGTGACCGTGGTCCC | 179 |

Primer Sequences Used to Amplify VL domains

| | | |
|---|---|---|
| Hu Vkappa1-5′ | GACATCCAGATGACCCAGTCTCC | 180 |
| Hu Vkappa2a-5′ | GATGTTGTGATGACTCAGTCTCC | 181 |
| Hu Vkappa2b-5′ | GATATTGTGATGACTCAGTCTCC | 182 |
| Hu Vkappa3-5′ | GAAATTGTGTTGACGCAGTCTCC | 183 |
| Hu Vkappa4-5′ | GACATCGTGATGACCCAGTCTCC | 184 |
| Hu Vkappa5-5′ | GAAACGACACTCACGCAGTCTCC | 185 |
| Hu Vkappa6-5′ | GAAATTGTGCTGACTCAGTCTCC | 186 |
| Hu Vlambda1-5′ | CAGTCTGTGTTGACGCAGCCGCC | 187 |
| Hu Vlambda2-5′ | CAGTCTGCCCTGACTCAGCCTGC | 188 |
| Hu Vlambda3-5′ | TCCTATGTGCTGACTCAGCCACC | 189 |
| Hu Vlambda3b-5′ | TCTTCTGAGCTGACTCAGGACCC | 190 |
| Hu Vlambda4-5′ | CACGTTATACTGACTCAACCGCC | 191 |
| Hu Vlambda5-5′ | CAGGCTGTGCTCACTCAGCCGTC | 192 |
| Hu Vlambda6-5′ | AATTTTATGCTGACTCAGCCCCA | 193 |
| Hu Jkappa1-3′ | ACGTTTGATTTCCACCTTGGTCCC | 194 |
| Hu Jkappa2-3′ | ACGTTTGATCTCCAGCTTGGTCCC | 195 |
| Hu Jkappa3-3′ | ACGTTTGATATCCACTTTGGTCCC | 196 |
| Hu Jkappa4-3′ | ACGTTTGATCTCCACCTTGGTCCC | 197 |
| Hu Jkappa5-3′ | ACGTTTAATCTCCAGTCGTGTCCC | 198 |
| Hu Vlambda1-3′ | CAGTCTGTGTTGACGCAGCCGCC | 199 |
| Hu Vlambda2-3′ | CAGTCTGCCCTGACTCAGCCTGC | 200 |
| Hu Vlambda3-3′ | TCCTATGTGCTGACTCAGCCACC | 201 |
| Hu Vlambda3b-3′ | TCTTCTGAGCTGACTCAGGACCC | 202 |
| Hu Vlambda4-3′ | CACGTTATACTGACTCAACCGCC | 203 |
| Hu Vlambda5-3′ | CAGGCTGTGCTCACTCAGCCGTC | 204 |
| Hu Vlambda6-3′ | AATTTTATGCTGACTCAGCCCCA | 205 |

PCR samples are then electrophoresed on a 1. 3% agarose gel. DNA bands of the expected sizes (−506 base pairs for VH domains, and 344 base pairs for VL domains) can be cut out of the gel and purified using methods well known in the art and/or described herein.

Purified PCR products can be ligated into a PCR cloning vector (TA vector from Invitrogen Inc., Carlsbad, Calif.). Individual cloned PCR products can be isolated after transfection of E. coli and blue/white color selection. Cloned PCR products may then be sequenced using methods commonly known in the art and/or described herein.

The PCR bands containing the VH domain and the VL domains can also be used to create full-length Ig expression vectors. VH and VL domains can be cloned into vectors containing the nucleotide sequences of a heavy (e. g., human IgG1 or human IgG4) or light chain (human kappa or human ambda) constant regions such that a complete heavy or light chain molecule could be expressed from these vectors when transfected into an appropriate host cell. Further, when cloned heavy and light chains are both expressed in one cell line (from either one or two vectors), they can assemble into a complete functional antibody molecule that is secreted into the cell culture medium. Methods using polynucleotides encoding VH and VL antibody domain to generate expression vectors that encode complete antibody molecules are well known within the art.

### Example 37

### Assays Detecting Stimulation or Inhibition of B Cell Proliferation and Differentiation

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL10 , IL-13, IL-14 and IL-15. Interestingly, these signals are by themselves weak effectors but can, in combination with various co-stimulatory proteins, induce activation, proliferation, differentiation, homing, tolerance and death among B cell populations.

One of the best studied classes of B-cell co-stimulatory proteins is the TNF-superfamily. Within this family CD40, CD27, and CD30 along with their respective ligands CD154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In Vitro Assay-Purified polypeptides of the invention, or truncated forms thereof, is assessed for its ability to induce activation, proliferation, differentiation or inhibition and/or death in B-cell populations and their precursors. The activity of the polypeptides of the invention on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 105 B-cells suspended in culture medium (RPMI 1640 containing 10% FBS, 5×10-5M 2ME, 100 U/ml penicillin, 10 ug/ml streptomycin, and 10-5 dilution of SAC) in a total volume of 150 ul. Proliferation or inhibition is quantitated by a 20 h pulse (1 uCi/well) with 3H-thymidine (6.7 Ci/mM) beginning 72 h post factor addition. The positive and negative controls are IL2 and medium respectively.

In Vivo Assay-BALB/c mice are injected (i.p.) twice per day with buffer only, or 2 mg/Kg of a polypeptide of the invention, or truncated forms thereof. Mice receive this treatment for 4 consecutive days, at which time they are sacrificed and various tissues and serum collected for analyses. Comparison of H&E sections from normal spleens and spleens treated with polypeptides of the invention identify the results of the activity of the polypeptides on spleen cells, such as the diffusion of peri-arterial lymphatic sheaths, and/or significant increases in the nucleated cellularity of the red pulp regions, which may indicate the activation of the differentiation and proliferation of B-cell populations. Immunohistochemical studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from mice treated with polypeptide is used to indicate whether the polypeptide specifically increases the proportion of ThB+, CD45R(B220)dull B cells over that which is observed in control mice.

Likewise, a predicted consequence of increased mature B-cell representation in vivo is a relative increase in serum Ig titers. Accordingly, serum IgM and IgA levels are compared between buffer and polypeptide-treated mice.

One skilled in the art could easily modify the exemplified studies to test the activity of polynucleotides of the invention (e.g., gene therapy), agonists, and/or antagonists of polynucleotides or polypeptides of the invention.

### Example 38

### T Cell Prolferation Assay

A CD3-induced proliferation assay is performed on PBMCs and is measured by the uptake of 3H-thymidine. The assay is performed as follows. Ninety-six well plates are coated with 100 (1/well of mAb to CD3 (HIT3a, Pharmingen) or isotype-matched control mAb (B33.1) overnight at 4 degrees C. (1 (g/ml in 0.05M bicarbonate buffer, pH 9.5), then washed three times with PBS. PBMC are isolated by F/H gradient centrifugation from human peripheral blood and added to quadruplicate wells (5×104/well) of mAb coated plates in RPMI containing 10% FCS and P/S in the presence of varying concentrations of polypeptides of the invention (total volume 200 ul). Relevant protein buffer and medium alone are controls. After 48 hr. culture at 37 degrees C., plates are spun for 2 min. at 1000 rpm and 100 (1 of supernatant is removed and stored −20 degrees C. for measurement of IL-2 (or other cytokines) if effect on proliferation is observed. Wells are supplemented with 100 ul of medium containing 0.5 uCi of 3H-thymidine and cultured at 37 degrees C. for 18-24 hr. Wells are harvested and incorporation of 3H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of polypeptides of the invention.

One skilled in the art could easily modify the exemplified studies to test the activity of polynucleotides of the invention (e.g., gene therapy), agonists, and/or antagonists of polynucleotides or polypeptides of the invention.

### Example 39

### Effect of Polypeptides of the Invention on the Expression of MHC Class II, Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and MHC class II antigens). Treatment with activating factors, such as TNF-, causes a rapid change in surface phenotype (increased expression of MHC class I and II, costimulatory and adhesion molecules, downregulation of FC(RII, upregulation of CD83). These changes correlate with increased antigen-presenting capacity and with functional maturation of the dendritic cells.

FACS analysis of surface antigens is performed as follows. Cells are treated 1-3 days with increasing concentrations of polypeptides of the invention or LPS (positive control), washed with PBS containing 1% BSA and 0.02 mM sodium azide, and then incubated with 1:20 dilution of appropriate FITC- or PE-labeled monoclonal antibodies for 30 minutes at 4 degrees C. After an additional wash, the labeled cells are analyzed by flow cytometry on a FACScan (Becton Dickinson).

Effect on the production of cytokines. Cytokines generated by dendritic cells, in particular L-12, are important in the initiation of T-cell dependent immune responses. IL-12 strongly influences the development of Thl helper T-cell immune response, and induces cytotoxic T and NK cell function. An ELISA is used to measure the IL-12 release as follows. Dendritic cells (106/ml) are treated with increasing concentrations of polypeptides of the invention for 24 hours. LPS (100 ng/ml) is added to the cell culture as positive control. Supernatants from the cell cultures are then collected and analyzed for IL-12 content using commercial ELISA kit(e.g., R & D Systems (Minneapolis, Minn.)). The standard protocols provided with the kits are used.

Effect on the expression of MHC Class II, costimulatory and adhesion molecules. Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of polypeptides of the invention or LPS (positive control), washed with PBS containing 1% BSA and 0.02 mM sodium azide, and then incubated with 1:20 dilution of appropriate FITC- or PE-labeled monoclonal antibodies for 30 minutes at 4 degrees C. After an additional wash, the labeled cells are analyzed by flow cytometry on a FACScan (Becton Dickinson).

Monocyte activation and/or increased survival. Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. Polypeptides, agonists, or antagonists of the invention can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×106/ml in PBS containing PI at a final concentration of 5 (g/ml, and then incubated at room temperature for 5 minutes before FACScan analysis. PI uptake has been demonstrated to correlate with DNA fragmentation in this experimental paradigm.

Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×105 cells/ml with increasing concentrations of the a polypeptide of the invention and under the same conditions, but in the absence of the polypeptide. For IL-12 production, the cells are primed overnight with WFN (100 U/ml) in presence of a polypeptide of the invention. LPS (10 ng/ml) is then added. Conditioned media are collected after 24 h and kept frozen until use. Measurement of TNF-alpha, IL-10, MCP-1 and IL-8 is then performed using a commercially available ELISA kit(e.g., R & D Systems (Minneapolis, Minn.)) and applying the standard protocols provided with the kit.

Oxidative burst. Purified monocytes are plated in 96-w plate at 2−1×105 cell/well. Increasing concentrations of polypeptides of the invention are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 nM PMA). The plates are incubated at 37(C. for 2 hours and the reaction is stopped by adding 20 μl in NaOH per well. The absorbance is read at 610 nm. To calculate the amount of H2O2 produced by the macrophages, a standard curve of a H2O2 solution of known molarity is performed for each experiment.

One skilled in the art could easily modify the exemplified studies to test the activity of polynucleotides of the invention (e.g., gene therapy), agonists, and/or antagonists of polynucleotides or polypeptides of the invention.

### Example 40

### The Effect of the NFkB Associated Polypeptides of the Invention on the Growth of Vascular Endothelial Cells

On day 1, human umbilical vein endothelial cells (HUVEC) are seeded at 2-5×104 cells/35 mm dish density in M199 medium containing 4% fetal bovine serum (FBS), 16 units/ml heparin, and 50 units/ml endothelial cell growth supplements (ECGS, Biotechnique, Inc.). On day 2, the medium is replaced with M199 containing 10% FBS, 8 units/ml heparin. A polypeptide having the amino acid sequence of 109-118, 126, 128, 144-152, or 160-161, and positive controls, such as VEGF and basic FGF (bFGF) are added, at varying concentrations. On days 4 and 6, the medium is replaced. On day 8, cell number is determnined with a Coulter Counter.

An increase in the number of HUVEC cells indicates that the polypeptide of the invention may proliferate vascular endothelial cells.

One skilled in the art could easily modify the exemplified studies to test the activity of polynucleotides of the invention (e.g., gene therapy), agonists, and/or antagonists of polynucleotides or polypeptides of the invention.

### Example 41

### Stimulatory Effect of Polypeptides of the Invention on the Proliferation of Vascular Endothelial Cells

For evaluation of mitogenic activity of growth factors, the colorimetric MTS (3-(4,5-dimethylthiazol-2-yl)-5-(3-carboxymethoxyphenyl)-2-(4-sulfophenyl)2H-tetrazolium) assay with the electron coupling reagent PMS (phenazine methosulfate) was performed (CellTiter 96 AQ, Promega). Cells are seeded in a 96-well plate (5,000 cells/well) in 0.1 mL serum-supplemented medium and are allowed to attach overnight. After serum-starvation for 12 hours in 0.5% FBS, conditions (bFGF, VEGF165 or a polypeptide of the invention in 0.5% FBS) with or without Heparin (8 U/ml) are added to wells for 48 hours. 20 mg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. In Vitro Cell. Dev. Biol. 30A:512-518 (1994).

One skilled in the art could easily modify the exemplified studies to test the activity of polynucleotides of the invention (e.g., gene therapy), agonists, and/or antagonists of polynucleotides or polypeptides of the invention.

### Example 42

### Stimulation of Endothelial Migration

This example will be used to explore the possibility that a polypeptide of the invention may stimulate lymphatic endothelial cell migration.

Endothelial cell migration assays are performed using a 48 well microchemotaxis chamber (Neuroprobe Inc., Cabin John, M D; Falk, W., et al., J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×105 cells suspended in 50 ul M199 containing 1% FBS are seeded in the upper compartment. The apparatus is then incubated for 5 hours at 37° C. in a humidified chamber with 5% CO2 to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

One skilled in the art could easily modify the exemplified studies to test the activity of polynucleotides of the invention (e.g., gene therapy), agonists, and/or antagonists of polynucleotides or polypeptides of the invention.

### Example 43

### Stimulation of Nitric Oxide Production by Endothelial Cells

Nitric oxide released by the vascular endothelium is believed to be a mediator of vascular endothelium relaxation. Thus, activity of a polypeptide of the invention can be assayed by determining nitric oxide production by endothelial cells in response to the polypeptide.

Nitric oxide is measured in 96-well plates of confluent microvascular endothelial cells after 24 hours starvation and a subsequent 4 hr exposure to various levels of a positive control (such as VEGF-1) and the polypeptide of the invention. Nitric oxide in the medium is determined by use of the Griess reagent to measure total nitrite after reduction of nitric oxide-derived nitrate by nitrate reductase. The effect of the polypeptide of the invention on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2KNO2+2KI+2H2SO4 62NO+I2+2H2O+2K2SO4

The standard calibration curve is obtained by adding graded concentrations of KNO2 (0, 5, 10, 25, 50, 100, 250, and 500 nmol/L) into the calibration solution containing KI and H2SO4. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×106 endothelial cells. All values reported are means of four to six measurements in each group (number of cell culture wells). See, Leak et al. Biochem. and Biophys. Res. Comm. 217:96-105 (1995).

One skilled in the art could easily modify the exemplified studies to test the activity of polynucleotides of the invention (e.g., gene therapy), agonists, and/or antagonists of polynucleotides or polypeptides of the invention.

### Example 44

### Suppression of TNF Alpha-Induced Adhesion Molecule Expression by a Polypeptide of the Invention

The recruitment of lymphocytes to areas of inflammation and angiogenesis involves specific receptor-ligand interactions between cell surface adhesion molecules (CAMs) on lymphocytes and the vascular endothelium. The adhesion process, in both normal and pathological settings, follows a multi-step cascade that involves intercellular adhesion molecule-1 (ICAM-1), vascular cell adhesion molecule-1 (VCAM-1), and endothelial leukocyte adhesion molecule-1 (E-selectin) expression on endothelial cells (EC). The expression of these molecules and others on the vascular endothelium determines the efficiency with which leukocytes may adhere to the local vasculature and extravasate into the local tissue during the development of an inflammatory response. The local concentration of cytokines and growth factor participate in the modulation of the expression of these CAMs.

Tumor necrosis factor alpha (TNF-a), a potent proinflammatory cytokine, is a stimulator of all three CAMs on endothelial cells and may be involved in a wide variety of inflammatory responses, often resulting in a pathological outcome.

The potential of a polypeptide of the invention to mediate a suppression of TNF-a induced CAM expression can be examined. A modified ELISA assay which uses ECs as a solid phase absorbent is employed to measure the amount of CAM expression on TNF-a treated ECs when co-stimulated with a member of the FGF family of proteins.

To perform the experiment, human umbilical vein endothelial cell (HUVEC) cultures are obtained from pooled cord harvests and maintained in growth medium (EGM-2; Clonetics, San Diego, Calif.) supplemented with 10% FCS and 1% penicillin/streptomycin in a 37 degree C. humidified incubator containing 5% CO2. HUVECs are seeded in 96-well plates at concentrations of 1×104 cells/well in EGM medium at 37 degree C. for 18-24 hrs or until confluent. The monolayers are subsequently washed 3 times with a serum-free solution of RPMI-1640 supplemented with 100 U/ml penicillin and 100 mg/ml streptomycin, and treated with a given cytokine and/or growth factor(s) for 24 h at 37 degree C. Following incubation, the cells are then evaluated for CAM expression.

Human Umbilical Vein Endothelial cells (HUVECs) are grown in a standard 96 well plate to confluence. Growth medium is removed from the cells and replaced with 90 ul of 199 Medium (10% FBS). Samples for testing and positive or negative controls are added to the plate in triplicate (in 10 ul volumes). Plates are incubated at 37 degree C. for either 5 h (selectin and integrin expression) or 24 h (integrin expression only). Plates are aspirated to remove medium and 100 p1 of 0.1% paraformaldehyde-PBS(with Ca++ and Mg++) is added to each well. Plates are held at 4oC. for 30 min.

Fixative is then removed from the wells and wells are washed 1× with PBS(+Ca,Mg)+0.5% BSA and drained. Do not allow the wells to dry. Add 10 μl of diluted primary antibody to the test and control wells. Anti-ICAM-1-Biotin, Anti-VCAM-1-Biotin and Anti-E-selectin-Biotin are used at a concentration of 10 μg/ml (1:10 dilution of 0.1 mg/ml stock antibody). Cells are incubated at 37oC. for 30 min. in a humidified environment. Wells are washed ×3 with PBS(+Ca,Mg)+0.5% BSA.

Then add 20 μl of diluted ExtrAvidin-Alkaline Phosphatase (1:5,000 dilution) to each well and incubated at 37oC. for 30 min. Wells are washed ×3 with PBS(+Ca,Mg)+0.5% BSA. 1 tablet of p-Nitrophenol Phosphate pNPP is dissolved in 5 ml of glycine buffer (pH 10.4). 100 μl of pNPP substrate in glycine buffer is added to each test well. Standard wells in triplicate are prepared from the working dilution of the ExtrAvidin-Alkaline Phosphatase in glycine buffer: 1:5,000 (100)>10-0.5>10-1>10-1.5. 5 μl of each dilution is added to triplicate wells and the resulting AP content in each well is 5.50 ng, 1.74 ng, 0.55 ng, 0.18 ng. 100 μl of pNNP reagent must then be added to each of the standard wells. The plate must be incubated at 37oC. for 4 h. A volume of 50 μl of 3M NaOH is added to all wells. The results are quantified on a plate reader at 405 nm. The background subtraction option is used on blank wells filled with glycine buffer only. The template is set up to indicate the concentration of AP-conjugate in each standard well [5.50 ng; 1.74 ng; 0.55 ng; 0.18 ng]. Results are indicated as amount of bound AP-conjugate in each sample.

One skilled in the art could easily modify the exemplified studies to test the activity of polynucleotides of the invention (e.g., gene therapy), agonists, and/or antagonists of polynucleotides or polypeptides of the invention.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

TABLE I

| | | | | Total | 5′ NT | | | |
|---|---|---|---|---|---|---|---|---|
| | | Genbank | NT SEQ | NT Seq | of Start | 3′ NT | | Total |
| Gene | Clone | Accession | ID. No. | of | Codon | of | AA Seq | AA of |
| No. | Name | No. | X | Clone | of ORF | ORF | ID No. Y | ORF |
| 1 | | Ac024562 | 1 | 588 | | | | |
| 2 | | Al158013 | 2 | 678 | | | | |
| 3 | | AP000780 | 3 | 567 | | | | |
| 4 | | Al355483 | 4 | 1026 | | | | |
| 5 | | Al137848 | 5 | 474 | | | | |
| 6 | | Al357992 | 6 | 529 | | | | |
| 7 | | Ac008435 | 7 | 454 | | | | |
| 7 | | Ac008435 | 264 | 455 | | | | |
| 8 | | Ac005625 | 8 | 247 | | | | |
| 9 | | Al354881 | 9 | 254 | | | | |
| 9 | | Al354881 | 265 | 912 | | | | |
| 10 | | Ac007014 | 10 | 3308 | | | | |
| 10 | | Ac007014 | 280 | 3329 | | | | |
| 11 | | Ac010791 | 11 | 755 | | | | |
| 11 | | Ac010791 | 281 | 2182 | | | | |
| 12 | | Al008730 | 12 | 393 | | | | |
| 13 | | Ac068709 | 13 | 359 | | | | |
| 14 | | Ac023602 | 14 | 643 | | | | |
| 14 | | Ac023602 | 266 | 1412 | | | | |
| 15 | | Ac0l1244 | 15 | 211 | | | | |
| 16 | | Ac026974 | 16 | 138 | | | | |
| 17 | | Ac026843 | 17 | 628 | | | | |
| 18 | | Al096868 | 18 | 403 | | | | |
| 19 | | Al136528 | 19 | 582 | | | | |
| 20 | | Ac011236 | 20 | 317 | | | | |
| 21 | | Ac008576 | 21 | 269 | | | | |
| 22 | | Al136163 | 22 | 354 | | | | |
| 23 | | Ac026314 | 23 | 368 | | | | |
| 24 | | Al354926 | 24 | 459 | | | | |
| 25 | | Ac004168 | 25 | 149 | | | | |
| 26 | | Ac068619 | 26 | 90 | | | | |
| 27 | | Ap002338 | 27 | 408 | | | | |
| 27 | | Ap002338 | 267 | 1925 | | | | |
| 28 | | Al158062 | 28 | 697 | | | | |
| 28 | | Al158062 | 268 | 2632 | | | | |
| 29 | | Al132777 | 29 | 179 | | | | |
| 30 | | Ac008762 | 30 | 277 | | | | |
| 31 | | Al157402 | 31 | 98 | | | | |
| 32 | | Ac022795 | 32 | 241 | | | | |
| 33 | | Ac015564 | 33 | 1880 | | | | |
| 33 | | Ac015564 | 269 | 1935 | | | | |
| 34 | | Ac022862 | 34 | 1199 | | | | |
| 35 | | Al035683 | 35 | 336 | | | | |
| 36 | 116917 | | 36 | 700 | | | | |
| 36 | 116917 | | 270 | 1302 | | | | |
| 37 | 22946 | | 37 | 855 | | | | |
| 38 | 206416 | | 38 | 544 | | | | |
| 39 | 1137189 | | 39 | 560 | | | | |
| 39 | 1137189 | | 271 | 581 | | | | |
| 40 | 7248 | | 40 | 467 | | | | |
| 40 | 7248 | | 279 | 2842 | | | | |
| 41 | 1101000 | | 41 | 1391 | | | | |
| 42 | 421725 | | 42 | 593 | | | | |
| 43 | 14249 | | 43 | 767 | | | | |
| 44 | 1336656 | | 44 | 1145 | | | | |
| 45 | 459363 | | 45 | 338 | | | | |
| 46 | 899587 | | 46 | 440 | | | | |
| 46 | 899587 | | 272 | 460 | | | | |
| 47 | 334519 | | 47 | 1098 | | | | |
| 48 | 185587 | | 48 | 1477 | | | | |
| 49 | 436375 | | 49 | 619 | | | | |
| 50 | 337323 | | 50 | 789 | | | | |
| 50 | 337323 | | 273 | 1335 | | | | |
| 51 | 251758 | | 51 | 1530 | | | | |
| 52 | 346607 | | 52 | 1310 | | | | |
| 52 | 346607 | | 274 | 1466 | | | | |
| 53 | 402834 | | 53 | 2538 | | | | |
| 54 | 328027 | | 54 | 763 | | | | |
| 55 | 213757 | | 55 | 934 | | | | |
| 56 | 404343 | | 56 | 838 | | | | |
| 56 | 404343 | | 275 | 2539 | | | | |
| 57 | 30507 | | 57 | 1319 | | | | |
| 57 | 30507 | | 276 | 1563 | | | | |
| 58 | 436679 | | 58 | 709 | | | | |
| 59 | 899656 | | 59 | 1187 | | | | |
| 60 | 386674 | | 60 | 408 | | | | |
| 61 | | Ac036181 | 61 | 2907 | | | | |
| 62 | | Ac040977 | 62 | 650 | | | | |
| 63 | | Ab014087 | 63 | 3853 | | | | |
| 64 | | Al136332 | 64 | 376 | | | | |
| 65 | | Ac010532 | 65 | 283 | | | | |
| 66 | | Ac010611 | 66 | 1574 | | | | |
| 67 | | Ac016461 | 67 | 430 | | | | |
| 68 | | Ac012357 | 68 | 677 | | | | |
| 69 | | Ac016008 | 69 | 554 | | | | |
| 70 | 242250 | | 70 | 1702 | | | | |
| 70 | 242250 | | 277 | 2683 | | | | |
| 71 | 331938 | | 71 | 567 | | | | |
| 72 | 215056 | | 72 | 1465 | | | | |
| 73 | 14359 | | 73 | 965 | | | | |
| 74 | | Ac024191 | 74 | 1807 | 1 | 1468 | 109 | 490 |
| 74 | | Ac024191 | 284 | 1775 | 1 | 1468 | 109 | 490 |
| 75 | | Ac022137 | 75 | 535 | 76 | | 110 | 153 |
| 76 | | Ac005027 | 76 | 2450 | 37 | 1190 | 111 | 385 |
| 77 | | Ac022694 | 77 | 2395 | 708 | 2322 | 112 | 568 |
| 78 | 235347 | | 78 | 5075 | 323 | 2258 | 113 | 645 |
| 78 | 235347 | | 282 | 5085 | 323 | 2258 | 113 | 645 |
| 79 | 360380 | | 79 | 2259 | 229 | 1078 | 114 | 284 |
| 80 | 246666 | | 80 | 1519 | 792 | 1374 | 115 | 195 |
| 81 | 204305 | | 81 | 3818 | 338 | 2771 | 116 | 812 |
| 82 | 899425 | | 82 | 2900 | 157 | 2170 | 117 | 672 |
| 83 | 283 | | 83 | 635 | | | | |
| 84 | 404 | | 84 | 1046 | | | | |
| 85 | 75 | | 85 | 284 | | | | |
| 86 | 110 | | 86 | 1632 | | | | |
| 87 | 70 | | 87 | 480 | | | | |
| 88 | 310 | | 88 | 1583 | | | | |
| 89 | 212 | | 89 | 742 | | | | |
| 90 | 67 | | 90 | 2729 | | | | |
| 91 | 371 | | 91 | 470 | | | | |
| 92 | 262 | | 92 | 597 | | | | |
| 92 | 262 | | 262 | 769 | | | | |
| 93 | 65 | | 93 | 1140 | | | | |
| 94 | 325 | | 94 | 520 | | | | |
| 95 | 297 | | 95 | 501 | | | | |
| 96 | 103 | | 96 | 1760 | | | | |
| 97 | 360 | | 97 | 217 | | | | |
| 98 | 151 | | 98 | 1311 | | | | |
| 99 | 17 | | 99 | 144 | | | | |
| 100 | 255 | | 100 | 528 | | | | |
| 101 | | Ac025631 | 101 | 1287 | | | | |
| 102 | 127 | | 102 | 3670 | 130 | 1657 | 118 | 510 |
| 109 | AD037 | | 125 | 2503 | 149 | 1111 | 126 | 321 |
| 110 | Cyclin L | NM_020307 | 127 | 2076 | 55 | 1632 | 128 | 526 |

TABLE II

| | | Genbank | NT SEQ ID: | AA | | |
|---|---|---|---|---|---|---|
| Gene No. | Clone Name | Accession No. | No. X | Seq ID No. Y | Polynucleotide Sequence | Polypeptide Sequence |
| 1 | | Ac024562 | 1 | | GGGACAGTGGTTCTTTCATTTCAATGATCAAAGTTC | |
| | | | | | CCAGCTTTTTGACACCACAGGGGCACCCTGACAATT | |
| | | | | | CTGGCAATAAGAACATGAAAGGCCTGGTCTTTATTT | |
| | | | | | CACTCAATTCCTGCTATGTGTGGTGAGTGTGGGTGA | |
| | | | | | GCCAAGGGGAAGGTGATCCTATTGTCAGGAGGTAA | |
| | | | | | TTTACCATGAATAGGGGATGATATGGAAATAATGT | |
| | | | | | GTGTGATCCTTCCCCTGCCACTGTTGGGATGTCTTTT | |
| | | | | | TAATTTCCTTCCCTCATTTGTCACAGCCGTGAAAAT | |
| | | | | | ACTTTTTCTGATATGATGAATGACAGATGGCAGGGT | |
| | | | | | GCCGGCAGCCCTTCTGGAGGGATGGGAGGTTGTGT | |
| | | | | | GTGTCCACGATAGGGGCCCAATAAGTACTGGCTGA | |
| | | | | | ATGAGAAAATGAGGAGCCTCACTGTGGGCTTTCTTT | |
| | | | | | GGGGTGAATGGAGGTGCTGAGTGACCTCTCAGCTT | |
| | | | | | CCTAGAAGTCACAGGCCAGAAGCCGTGGAATCTCA | |
| | | | | | GTGGTGGAAAGTCCTACTGATTTGAGGATCAGGGA | |
| | | | | | GGGAGAGAATCAGCAATGGTGTGCTGATAAATGTT | |
| | | | | | TAGTAGTTGGCTCTCTGGT | |
| 2 | | A1158013 | 2 | | ATTTCATTAATGTTTGATTGAAAGTAAATTGAAGTG | |
| | | | | | TAGCTCAAGGTGGATCATACACATAGCAACATTATT | |
| | | | | | GCAGAGGAATTATTGCCATTTAGGTAATAGAGCAA | |
| | | | | | TGGAATCAAAATAAAATACTGATTATATGGATTGAT | |
| | | | | | GGAGCTTTTTAAATTTAATGCTGATTTCAAAATGTT | |
| | | | | | TTGATGATTATTTGGCAAGTGAGTGTTTGTATGTTA | |
| | | | | | CGCTAAAAGAGGATTTTCCCCCCTAAGATGCAGCTC | |
| | | | | | ACCATAAGAAAGGTTGTATACTATTTGTATATGAAA | |
| | | | | | TCTGGTCTCCCAACATCAACTGAGAAAATAAATAA | |
| | | | | | CCCTATCCTTCTGTAAACATGGTATTTACTCTCTTTG | |
| | | | | | AGGTATTTTCTTGTCTGAATTTGAATACCTTGATAA | |
| | | | | | AGTACTAGAACAAACAAGTAAAATTTAAAATTG | |
| | | | | | ACATCAATTAATCTATATTCAAAGCATGACAAGAA | |
| | | | | | GAAGAAAGGTGATTTATTGAATTGTAATCAAGATA | |
| | | | | | TAAGGAATAAGTAACTACAATATAATTTTTCCACCA | |
| | | | | | TATTTAGAACTTAGGAGTTGCACTGGTTTTGTTGGT | |
| | | | | | GTTTTATTGTACAAATAATGTATTTACTCTTTAATAT | |
| | | | | | GCCGATTTATATTTCCTATGTTTCTAATGGATATTTA | |
| | | | | | AATATAACTTAAAAGAAACAAGTTCTTTTTTC | |
| 3 | | AP000780 | 3 | | GCCCTGTGAGAAGAGAAGTCTTTTCTCTGACCAGAT | |
| | | | | | GTCATCTTTCCTTTTCTAATACTTCAGGTCTTATGCC | |
| | | | | | CTGTTGTATGAGTGGCATAGTTCATTGATCTTATCA | |
| | | | | | CAGGAAATCAGTGCCTTGAGTATACGTATATGGTTG | |
| | | | | | TTGAAAGAATTCAGTTCAGTTCATGTTATCAGACAT | |
| | | | | | CATAAATGAAAAATCTTCAGTGTCGTAAAGGATAG | |
| | | | | | GAAGTGTTAATTTCTCCTTTTTACTCTTGTGACTTTT | |
| | | | | | CTAGAGGGTCCTTATATATTGGGGCAATTTTTAAAT | |
| | | | | | TACAATTAAAAAAATACCTAGCTTAGGCTGGGTGC | |
| | | | | | GTCGGCTCAAGCTTGTAATCCCAGCACTTTGGGAGG | |
| | | | | | CCGAGGTGGGTGGATCACTTGAGGTCAGAAGTTCG | |
| | | | | | AGACCAGGCTGGCCATCACGGTGAAACCCTGTCTC | |
| | | | | | CATTAAAAATACAAAAATTGGCCAGGCGCGGTGGC | |
| | | | | | TCACGCCTGTAATCCCAGCACTTTGGGAGGCCAACA | |
| | | | | | TGGGTGGATCACGAGGTCAGGAGATCGAGACCATC | |
| | | | | | CTGGCTAACACGGTGAAAACCCATCTCTACT | |
| 4 | | A1355483 | 4 | | TGCATTCACACATCCCAGTCACGATGACAGTAAAGT | |
| | | | | | GTGGCTTGCAGGCTGTGCTGGGGCCTCTCTTCCTTT | |
| | | | | | CCAGGCGTCCCTCTTTGCCAGCACCTGCTAGTGGGT | |
| | | | | | GTGCCAACTCCCTCCTGAGCAGCCCAGCCCCTTGGG | |
| | | | | | CGCCCTCCAGCATGAGCTGGGTCCCCCGGCAGCGG | |
| | | | | | TTTTAATTATCAGCCCTGCTCACCCCAGCTCCTCTCA | |
| | | | | | CAAGCTGCCATATGTCATAGACTCCAGTAATCACCC | |
| | | | | | CGCAGCCGGAGTGGCAGGGGAGGGGCTGAGGGCCT | |
| | | | | | TCAGGGGAATCCTGCTCAGTCTTGACCGAGTTCCTC | |
| | | | | | ACTGACTGTACCCGCTCTGACCTCTTTGTCTCTGGT | |
| | | | | | GGGGCCCAGCCTAGGTACCCACAATGGGAGAGCCG | |
| | | | | | GGCCTAGCTGCTTTGGGGGCATAGAATGCGGCATG | |
| | | | | | CTCTCAGGCGCCATGGAGTGTCCTTGGGAAACTGA | |
| | | | | | GAGTCACCCAGCGAGCCCAGGGCTGTGGGGCTCAT | |
| | | | | | GTGGTGCACACAGTTCCCATGACCCCTCATGGCCTC | |
| | | | | | TACACGCCTGCCCCTTGGAACGTGGCATGTGGCAG | |
| | | | | | GACAGACACCCCAAAGCTGTCTGCCAGTCTGTCTAG | |
| | | | | | GAGTCCACGGGAGTGGTCATTTGGCCCCCATCCTCC | |
| | | | | | CCTGGTCACTGGCCTTGAGGTACCACAGGGGACTTC | |
| | | | | | ATCCCAGCCACTCTGGAGGGCATCTTAGTTTCCAGC | |
| | | | | | CCTCTCAACCTGCCGTAATCCTTGGATGGCTTTTCC | |
| | | | | | AGTTGGTGCCTCACAGGTGTGCTCCTGGGAGGCAG | |
| | | | | | GCGGTGCAGGAGTTCATTATGATCCCCATTCCTTGA | |
| | | | | | TGAGGAAAACGAGGCTCAGAGAGGATAAGAGACTC | |
| | | | | | ACCCAGTTATTGGTAGTTCTGGAGCTAAAACTCACT | |
| | | | | | TCAACTGATTTTACTTATTTAGTTTTCCAGGGTAAGT | |
| | | | | | AACTTCTGGTTAGCTGAAAGTAACTTTACACTTGTA | |
| | | | | | ATGAAAAACATAGTTAATAAAGAACAGGAAACGAA | |
| | | | | | GGTTGCAGTGAGCCGAGATCACACCA | |
| 5 | | A1137848 | 5 | | AAAGCAAAACAAAACAAAGACTTAAAAGATATATC | |
| | | | | | AACTTATGACATCTGTGTGGGCCTTATGTGGATACT | |
| | | | | | GACTCAACAGACAAACGAGTTTAAAAATTGTGGAA | |
| | | | | | CAGTTGGCAAGTTGAACATTTGCTGGGTTTGATGAT | |
| | | | | | AGTAAGGAAATATTGTCAATTATTTTTTGGTATGGT | |
| | | | | | AATTGTATTGTAGTTAATGTTTTAAAAAGTAGAGAG | |
| | | | | | AGGTATTCTTTCTAAGGCCGAAATAACCCCTACCCC | |
| | | | | | AAAATTTGACAGGTGCATCACAAGAAAATAGAATT | |
| | | | | | ACAGTCCAGTAAACACACAAATAGTAAATAAAACA | |
| | | | | | TTATAAGTTAAAATTTAGATATATATAAAAACAAG | |
| | | | | | GCCGGGCACAGTGGCTCACACCTGTAATCCCAGAA | |
| | | | | | CTGTGGGAGGCCAAGGCGGGCAAATCTCCTGAGGT | |
| | | | | | CAGGAGTTCGAGACCAGCCTGACCAACATGGAGAA | |
| | | | | | ACCCCGTCTCTACT | |
| 6 | | A1357992 | 6 | | GCCTCCCAGGTTCAAGCAATTCTCCTGTCTCAGCCT | |
| | | | | | CCAGAGTAGCTGGGATTACAGGCACCTGCCACCAT | |
| | | | | | GCCCAGTTGATTTTTTGTATTTTTAGTAGAGATGGG | |
| | | | | | GTTTCACTATGTTGGCCAGGCTGGTCTTGAACTCCT | |
| | | | | | GACCTCGTGATCCACCCACCTTGGCCTCCCAAAGTG | |
| | | | | | CTGGGATTACAGGTGTGAGCCACCACGCCTGGACTT | |
| | | | | | TTTTTTTTGTATTTTTAGTAGAGACGAGCTTTTGCTA | |
| | | | | | TGTTGCTCAGGCTAGTCTCAAACTCCTAGCCTCAAG | |
| | | | | | TGATCTGTCTGCCTTGGCTTCCCAAAATGGTAGGAT | |
| | | | | | TACAGGTGCAAGTCACTATACCTGGCCTCAGTTTCT | |
| | | | | | CATTTTTAAAAGGTGATAAGTAATAAACAAACATA | |
| | | | | | ATAAGGATTAATCAATAAAAAATAATTATGTATAA | |
| | | | | | GATGACATATGTGATCATATGTAATAATTATGTATA | |
| | | | | | TGTTCAACCAGTGAGGTTGCTTCTACCGAGTAAACC | |
| | | | | | TGCTGGGGCCTTGGTGCTCCCTAATTC | |
| 7 | | Ac008435 | 7 | | CTACAAGTGGTCAAAGATCTACCTGTAACTGTCTAG | |
| | | | | | ATATTTGCCTCTAAATAATGAGACAATGCGAATGCA | |
| | | | | | AAGAGCCAGTATGATTAAGAATATGACCATTTTCA | |
| | | | | | GAAAAAGCATATTGACTCTCTTGGGTCAGATATGGT | |
| | | | | | GGCTCACACCTATAATCCCAGTACTATGGGAGGCTG | |
| | | | | | AGGCTGGAGAATCTCTTGAGGCCAGGAGTTTGAGA | |
| | | | | | ACAGCCTGGGCAACATGGTGAAACCCTGCCTCTCTA | |
| | | | | | CAAAAGTAAATTAAATAAATGAAAATTTTCACACA | |
| | | | | | GATTAAGAGTTTATTTAAAAATATCTTTCTCATAAA | |
| | | | | | TACTAGTTAATTTCTTTTCACTTATGAAATTTTTTAT | |
| | | | | | AGTAATTTATACTTTTGGTTCAGGCAAGCTGTGTTC | |
| | | | | | ATTTTGATTTAAAGTAATTCCTATAGGTGTTTTGACT | |
| | | | | | TTTCTAGACTATAAGACCTGTGT | |
| 7 | | Ac008435 | 264 | | CTACAAGTGGTCAAAGATCTACCTGTAACTGTCTAG | |
| | | | | | ATATTTGCCTCTAAATAATGAGACAATGCGAATGCA | |
| | | | | | AAGAGCCAGTATGATTAAGAATATGACCATTTTCA | |
| | | | | | GAAAAAGCATATTGACTCTCTTGGGTCAGATATGGT | |
| | | | | | GGCTCACACCTATAATCCCAGTACTATGGGAGGCTG | |
| | | | | | AGGCTGGAGAATCTCTTGAGGCCAGGAGTTTGAGA | |
| | | | | | ACAGCCTGGGCAACATGGTGAAACCCTGCCTCTCTA | |
| | | | | | CAAAAGTAAATTAAATAAATGAAAATTTTCACACA | |
| | | | | | GATTAAGAGTTTATTTAAAAATATCTTTCTCATAAA | |
| | | | | | TACTAGTTAATTTCTTTTCACTTATGAAATTTTTTAT | |
| | | | | | AGTAATTTATACTTTTGGTTCAGGCAAGCTGTGTTC | |
| | | | | | ATTTTGATTTAAAGTAATTCCTATAGGTGTTTTGACT | |
| | | | | | TTTCTAGACTATAAGACCTGTGTA | |
| 8 | | Ac005625 | 8 | | CAGAGGGAGAGGGGCATGGCAAATCAGAAAGACA | |
| | | | | | GAGCGGGAGGAGAGAGAGAAACAGATGGGCAAAG | |
| | | | | | CCTCAAGGGAAACTCATTGGAGAGGAAAAAAGAGA | |
| | | | | | GTCTAGGCACAGTGGCTCAGGAGGCCAGACTATTC | |
| | | | | | AAGAGGCTGACGGGAGGGGGCATCGCATGAGCCCA | |
| | | | | | GGGGTTTGAGGCTGCAGTGAGCTATGATCACACCA | |
| | | | | | CTGCACTCCAGCCTGGGCGACAGAGCAAGACCCTG | |
| | | | | | TTCC | |
| 9 | | A1354881 | 9 | | TGGTCCTTGATGTCGATATTCTTAACACTCTTTGATG | |
| | | | | | GGTAAGAAAATTAAGACTATCAAAGGTAACAGAAA | |
| | | | | | AGAAGTAAATGGCAACTAAAGCATGGAAAGTGAGT | |
| | | | | | TTTATAAAGAAAGTAAAAAAAAAAAAAACAAGTGC | |
| | | | | | AAATATCCATACTTCAATTGTGACTCAAAGCCAACA | |
| | | | | | TGACTCTGTCTACATTTCAGCATCTCACTTAAGATT | |
| | | | | | CTTGAAGAGGGTAAGCTGATACTCAAGAAGAATTA | |
| | | | | | GTCTT | |
| 9 | | A1354881 | 265 | | GATCTATCCTTTTAACTCTTAAAATGGTCCTTGATGT | |
| | | | | | CGATATTCTTAACACTCTTTGATGGGTAAGAAAATT | |
| | | | | | AAGACTATCAAAGGTAACAGAAAAGAAGTAAATGG | |
| | | | | | CAACTAAAGCATGGAAAGTGAGTTTTATAAAGAAA | |
| | | | | | GTAAAAAAAAAAATAACAAGTGCAAATATCCATAC | |
| | | | | | TTCAATTGTGACTCAAAGCCAACATGACTCTGTCTA | |
| | | | | | CATTTCAGCATCTCACTTAAGATTCTTGAAGAGGGT | |
| | | | | | AAGCTGATACTCAAGAAGAATTAGTCTTTATATTTA | |
| | | | | | GCCTCTTTTTTCTCATTGTTATCACAAATTGGTTTTCT | |
| | | | | | TTTAGTTACCATCAGAAAATAATATTTTTTTAAATG | |
| | | | | | TGAAGATTCCCAAATATTATAACAGACAAATAACA | |
| | | | | | GATAATTATAATTTAAAAAATCCATCGAGAGATTGT | |
| | | | | | GGTATTAAATTTGCTACAGAGTGGCCTTTAGTTAAA | |
| | | | | | TCTCTCATGCCTTCACAAAGCCAGAATTATTCTCTA | |
| | | | | | TAAAAGTTATTCTTAATTGGCTTCTTAATCAGGAAT | |
| | | | | | TTTTAAATTGTACATAGTTGTGTATACTTTCTATTTA | |
| | | | | | TTCAGAGGAAAATGCAATTAAGTTTTTAGACCATTT | |
| | | | | | GCTTTACTTCTGTCCCCAGATAAAAATGTAAATTGT | |
| | | | | | TTAGTCACTATCCACAACTATGAATAGATTATTTTA | |
| | | | | | AAAAATAAACCTGACTTAATTTTAAGCAAAAAGCA | |
| | | | | | AGTCTTGAGTATTTTGCCAATCTACTTTTTTTAATTT | |
| | | | | | GTAATATTGTTTAATCTACTGTCACTTGTAAGTACTT | |
| | | | | | CGGTGTAATTGTAAAATGGCTCCCAAGATTTTGGAA | |
| | | | | | ATGAGGGAAATAGAATTCCAGCTGGAGAGTTCATTA | |
| | | | | | AATCATTACTTAATGAGTTCATGCAAAGCCCCAGAA | |
| | | | | | GGAGATAAAATAG | |
| 10 | | Ac007014 | 10 | | CCCTGCGCTGTCGGGCGGGGAGGTCGGAAACCCCC | |
| | | | | | TGGCGAGACCACGGGCGGACGCTTCCCGAAGAGCT | |
| | | | | | GCCTGGGCTGCAGCCGCGGAAGCTGCGTTCTGGGG | |
| | | | | | AGCGGGGAGCGTGCTCCGGCGCCTTCGGGCCGCTG | |
| | | | | | CTGGAAGCCGGAACCGAGCCCGGGCCGCTGCCCCT | |
| | | | | | CACCGGACGCCGCGCGCCACCGGCCCTCCGCGGGG | |
| | | | | | CAGGGGCTGCTGCGAGCTCGCCGGGCGCCCTTTAG | |
| | | | | | ACAGTCGTCCTTGTCTACTCCACTACCAAATGTTGA | |
| | | | | | AGTTCTTCAAGAATCAGTCCTTTGGAGGTGATGTCA | |
| | | | | | TTGAAAATGATGAGTAGGAAACTCCAAGAGCGCAT | |
| | | | | | TTCTCCACAAAACCAGTGAATACATTGGCACAAATT | |
| | | | | | GTCAGAATCAATTTTATATAAATTCTGGAAATTAGT | |
| | | | | | CAAAGGTTTATAGTAACCAAGGAAACATCTTTTTAA | |
| | | | | | AAAGATGGCTGAGTGGACCTTCTTTTCAAAGAATTA | |
| | | | | | TGGAGGCTTATTTTAGTTCCCCTAACTTGGAAATCT | |
| | | | | | CCTGAGGAAGAAAGGTGACTACAGGCATTTGTCAA | |
| | | | | | AAATTTGTAAAGGCAAGTTTATTAGCCTCTGCCATC | |
| | | | | | GGGGGCAAAGAATAATAGCTAAGGCAAACAATAG | |
| | | | | | ACACACCAAAAAGCCTGGGAGGAAAAGCTGGAAA | |
| | | | | | GTAAGATATTTTGGAGAATAAAGGCTTTTAAAACTT | |
| | | | | | CCACATATTCTTGGGAATCCAAAAGGCCACATGTAC | |
| | | | | | ATGCAGGGTGAGCAAATAGAGAAGACTTGAGAAAG | |
| | | | | | CCTTAAACTCTCACCTCTGGCTAACCATGAGGCTTG | |
| | | | | | CTCAAATAGGAAGTGAAAACTAAGGTGAAATTGTT | |
| | | | | | GCTTAGCTGAATGTTGAAGGTGTGCCCCAACACTTA | |
| | | | | | CACAGAGCCTACTGGTAAAGACAGAGTGTTTTCTTT | |
| | | | | | TTGTCTTGGTTTCAGGCATTTAAGGAAATCTTTCT | |
| | | | | | CTTTTGGATCACTAGCTGCAAATTAAGCTAACAGAA | |
| | | | | | CAGGAGCTCAGCTGGTCACACACAGCAACGAATAC | |
| | | | | | AGACTTTATAAAGTTCAGAAAAGTTACCAAACAGT | |
| | | | | | GGTAACCATAACAAGTACCAACAATGAACTATGGG | |
| | | | | | GAGGGAGGAGAATCTGATTTCCAGAGTTACCACAT | |
| | | | | | TATAATACTATTCAAAATGTCACATTTTTAGCAAAG | |
| | | | | | ATTACATGACAAGGAAAAACCAGAAAAGTATGGCC | |
| | | | | | CATACACAGGTAAAAAAAGAAATTAATAGAAACTA | |
| | | | | | CCCCTGAAGAAGCACAGACTTCGGATGTACAAAAC | |
| | | | | | AAAGACTTTTCATCAACTCTTTTAGATATGCTAGAA | |
| | | | | | GAGCTAAAGGAAACCATGGACAGAGAACAAAAAA | |
| | | | | | ATTAGGAAAGCAATGTCTCATCCAATACAGAATAT | |
| | | | | | CAATAAAGAGATTGAAATTGTAGAAAAGAACCAAA | |
| | | | | | TAGAAATTCTGGAGTTGAAAAGTATTATAACTAAA | |
| | | | | | ACTGAAAATTCACTAGAGGTATTCAGCAGCAGACT | |
| | | | | | GGAGAAGTCAGAAGAAAGAATCAACAGGCTTCAAG | |
| | | | | | ATAGGTCAATTAAGATTATACAGTCTGAGGAGCAG | |
| | | | | | AAAGGAAAAAGAATGAAGAAAAATGAACAGAGCA | |
| | | | | | TAAAAGACCTCTGGGACTCTATCAAGCATACCAGT | |
| | | | | | ATATGCATGAGGGGAGTCCCAGAAGGAGAAGAAA | |
| | | | | | GAGAGAAAGGGACATAATATTTGAAGAAATAATGG | |
| | | | | | TAGAAAATGTCCCAGCTTTGATGAAATACATGAATC | |
| | | | | | TAGATATTCAAGAGGCTCAAAGAACCCTAAATAGG | |
| | | | | | GTAAACTCAAAAAGACCCACACCGGAATGCAAAAG | |
| | | | | | TGAGCTGGGTGTGGTGGCACGTGCCTGTGGTCCCAG | |
| | | | | | CTACTCGAGAGGCTAAGGCAGGAAAATCGCTTGAA | |
| | | | | | CCCAGGAGGCAGAGATTGCGGTGAGCCGGGATTGC | |
| | | | | | GCCAGTGCACTCCAGCTGGGCGACAGAGCGAGATT | |
| | | | | | CCATCTCGCTATTGCTGCAGTCATTCAGATGGAAAT | |
| | | | | | GGGGAAAGAATAATATTAACTGATTTCAAAAAGGA | |
| | | | | | CTTGAAGATGTGAATCATCTATTTTGCTGAAGAAAT | |
| | | | | | CTTAACTCTTTGAAATTACTTTTTGTTGCTGTTGTCA | |
| | | | | | TACTCTTAGGTGCCAAACTGCGGTAAATTTTTTATC | |
| | | | | | AGTGAAGTGGAAGCATGTGTTTTGTTGTTTTGGGAA | |
| | | | | | TTTTTATCAAGTATCTTCAGAGAAGATTATTTCCTG | |
| | | | | | CTTTATCTTCAAAAACTGGAAAGGAAGGGTCAAAG | |
| | | | | | AAAAGACAGTAGCTGGCCGGTCATGGTGGCTCATG | |
| | | | | | CCTGTAATCCCAACACTTTGGGAGGCTGAGGTGGG | |
| | | | | | CAGATCACCTGAGGTTGGGAGTTCGAGGCCAGCCT | |
| | | | | | GACCAACGTGGAGAAATGCCATCTCTACTAAAGAT | |
| | | | | | GCAAGGATTGGCCGGGCATGGTGGCGCGTGCCTGT | |
| | | | | | GATCCCAGCTGCTCAGGAGGCTGAGGCAGGAGAAT | |
| | | | | | CGCTTGGACCTGGGAGGTGGAGGTTGCGGTGAGCT | |
| | | | | | GAGATCACGCCATTGCACTCCAGCCTGGGCAACAA | |
| | | | | | GCGAAACTCTGTCTCAAAAAAAAAAGAAAAGACAG | |
| | | | | | TAGCTTATGTTCATGTCAAGCACCTCTCATCACAGT | |
| | | | | | CTAGTTCCAAGGAAAAAATTCCCAGCGTTTTCTACA | |
| | | | | | TTCGGTGCTGCGTCATCTGAAATCGGCACATTCCAT | |
| | | | | | GGAGGAAGGAGTCCTGCTTTGTTGCATGTATCCTAG | |
| | | | | | GGTTTAATGTTGGTAAATGAGTCACTCTAGCATTTG | |
| | | | | | TAGAAGGCTCCCTGAGACTCCTGCAGCAGTCGACC | |
| | | | | | AAGCCCAAGGACATAATTGAATCTGGAGAGTCCTG | |
| | | | | | GGGCCTTGTTTTGAAAAAGACTTGAAATACACATA | |
| | | | | | GGAAGAAAGGCATAAAAATAAATGTTCACTTGTCT | |
| | | | | | CTGCTGTGAGTATGTGTTCCAACTTTTCAGTGATGG | |
| | | | | | CTTTGAGAATTCTCAAACTTGACTGGCTCTAAGTGT | |
| | | | | | ATCTGGTGGCTTTTGTATCGTAACCTGAAACTGGCT | |
| | | | | | TAGTACTTTTTCCTAAAAGCTCAGGATTTGAGAATG | |
| | | | | | AGGACCCCTTCGCCAGGAAAACATGTATACACTCA | |
| | | | | | AAATTTTGCTTGCAGTTCTAGGGTGTTTAGACCTTT | |
| | | | | | CTCAGATACCTGTGCATCTTATGGGTTTTGTTTTTCT | |
| | | | | | CTTTGAGACAGTCTCACCCTGTTGCCCAGGCTGGAG | |
| | | | | | TGCAGTGGCATGGTCTCAGCTCATTGCAGCCTCCGC | |
| | | | | | CTCCTGGGTTCAGGTGGTTCTGCCTCAGCCCCTTGA | |
| | | | | | TCGGCTGGGATTGCATGCATGTGCCACCATGCCCGG | |
| | | | | | CTGATTTTTGTATTTTTAGTGGAGATGGAGACAGAG | |
| | | | | | TTTCACCATGTTGG | |
| 10 | | AcO07014 | 280 | | CCCTGCGCTGTCGGGCGGGGAGGTCGGAAACCCCC | |
| | | | | | TGGCGAGACCACGGGCGGACGCTTCCCGAAGAGCT | |
| | | | | | GCCTGGGCTGCAGCCGCGGAAGCTGCGTTCTGGGG | |
| | | | | | AGCGGGGAGCGTGCTCCGGCGCCTTCGGGCCGCTG | |
| | | | | | CTGGAAGCCGGAACCGAGCCCGGGCCGCTGCCCCT | |
| | | | | | CACCGGACGCCGCGCGCCACCGGCCCTCCGCGGGG | |
| | | | | | CAGGGGCTGCTGCGAGCTCGCCGGGCGCCCTTTAG | |
| | | | | | ACAGTCGTCCTTGTCTACTCCACTACCAAATGTTGA | |
| | | | | | AGTTCTTCAAGAATCAGTCCTTTGGAGGTGATGTCA | |
| | | | | | TTGAAAATGATGAGTAGGAAACTCCAAGAGCGCAT | |
| | | | | | TTCTCCACAAAACCAGTGAATACATTGGCACAAATT | |
| | | | | | GTCAGAATCAATTTTATATAAATTCTGGAAATTAGT | |
| | | | | | CAAAGGTTTATAGTAACCAAGGAAACATCTTTTTAA | |
| | | | | | AAAGATGGCTGAGTGGACCTTCTTTTTCAAAGAATTA | |
| | | | | | TGGAGGCTTATTTTAGTTCCCCTAACTTGGAAATCT | |
| | | | | | CCTGAGGAAGAAAGGTGACTACAGGCATTTGTCAA | |
| | | | | | AAATTTGTAAAGGCAAGTTTATTAGCCTCTGCCATC | |
| | | | | | GGGGGCAAAGAATAATAGCTAAGGCAAACAATAG | |
| | | | | | ACACACCAAAAAGCCTGGGAGGAAAAGCTGGAAA | |
| | | | | | GTAAGATATTTTGGAGAATAAAGGCTTTTAAAACTT | |
| | | | | | CCACATATTCTTGGGAATCCAAAAGGCCACATGTAC | |
| | | | | | ATGCAGGGTGAGCAAATAGAGAAGACTTGAGAAAG | |
| | | | | | CCTTAAACTCTCACCTCTGGCTAACCATGAGGCTTG | |
| | | | | | CTCAAATAGGAAGTGAAAACTAAGGTGAATTTGTTT | |
| | | | | | GCTTAGCTGAATGTTGAAGGTGTGCCCCAACACTTA | |
| | | | | | CACAGAGCCTACTGGTAAAGACAGAGTGTTTTCTTT | |
| | | | | | TTGTCTTGGTTTCAGGCATTTAAGGAAATCTGTTTCT | |
| | | | | | CTTTTGGATCACTAGCTGCAAATTAAGCTAACAGAA | |
| | | | | | CAGGAGCTCAGCTGGTCACACACAGCAACGAATAC | |
| | | | | | AGACTTTATAAAGTTCAGAAAAGTTACCAAACAGT | |
| | | | | | GGTAACCATAACAAGTACCAACAATGAACTATGGG | |
| | | | | | GAGGGAGGAGAATCTGATTTCCAGAGTTACCACAT | |
| | | | | | TATAATACTATTCAAAATGTCACATTTTTAGCAAAG | |
| | | | | | ATTACATGACAAGGAAAAACCAGAAAAGTATGGCC | |
| | | | | | CATACACAGGTAAAAAAAGAAATTAATAGAAACTA | |
| | | | | | CCCCTGAAGAAGCACAGACTTCGGATGTACAAAAC | |
| | | | | | AAAGACTTTTCATCAACTCTTTTAGATATGCTAGAA | |
| | | | | | GAGCTAAAGGAAACCATGGACAGAGAACAAAAAA | |
| | | | | | ATTAGGAAAGCAATGTCTCATCCAATACAGAATAT | |
| | | | | | CAATAAAGAGATTGAAATTGTAGAAAAGAACCAAA | |
| | | | | | TAGAAATTTCTGGAGTTGAAAAGTATTATAACTAAA | |
| | | | | | ACTGAAAATTCACTAGAGGTATTCAGCAGCAGACT | |
| | | | | | GGAGAAGTCAGAAGAAAGAATCAACAGGCTTCAAG | |
| | | | | | ATAGGTCAATTAAGATTATACAGTCTGAGGAGCAG | |
| | | | | | AAAGGAAAAAGAATGAAGAAAAATGAACAGAGCA | |
| | | | | | TAAAAGACCTCTGGGACTCTATCAAGCATACCAGT | |
| | | | | | ATATGCATGAGGGGAGTCCCAGAAGGAGAAGAAA | |
| | | | | | GAGAGAAAGGGACATAATATTTGAAGAAATAATGG | |
| | | | | | TAGAAAATGTCCCAGCTTTGATGAAATACATGAATC | |
| | | | | | TAGATATTCAAGAGGCTCAAAGAACCCTAAATAGG | |
| | | | | | GTAAACTCAAAAAGACCCACACCGGAATGCAAAAG | |
| | | | | | TGAGCTGGGTGTGGTGGCACGTGCCTGTGGTCCCAG | |
| | | | | | CTACTCGAGAGGCTAAGGCAGGAAAATCGCTTGAA | |
| | | | | | CCCAGGAGGCAGAGATTGCGGTGAGCCGGGATTGC | |
| | | | | | GCCAGTGCACTCCAGCTGGGCGACAGAGCGAGATT | |
| | | | | | CCATCTCGAAAAAAAAAAAAAACAAAAAACTATTG | |
| | | | | | CTGCAGTCATTCAGATGGAAATGGGGAAAGAATAA | |
| | | | | | TATTAACTGATTTCAAAAAGGACTTGAAGATGTGA | |
| | | | | | ATCATCTATTTTGCTGAAGAAATCTTAACTCTTTGA | |
| | | | | | AATTACTTTTTGTTGCTGTTGTCATACTCTTAGGTGC | |
| | | | | | CAAACTGCGGTAAATTTTTTATCAGTGAAGTGGAAG | |
| | | | | | CATGTGTTTTGTTGTTTGGGAATTTTTATCAAGTAT | |
| | | | | | CTTCAGAGAAGATTTATTTCCTGCTTATCTCAAAA | |
| | | | | | ACTGGAAAGGAAGGGTCAAAGAAAAGACAGTAGC | |
| | | | | | TGGCCGGTCATGGTGGCTCATGCCTGTAATCCCAAC | |
| | | | | | ACTTTGGGAGGCTGAGGTGGGCAGATCACCTGAGG | |
| | | | | | TTGGGAGTTCGAGGCCAGCCTGACCAACGTGGAGA | |
| | | | | | AATGCCATCTCTACTAAAGATGCAAGGATTGGCCG | |
| | | | | | GGCATGGTGGCGCGTGCCTGTGATCCCAGCTGCTCA | |
| | | | | | GGAGGCTGAGGCAGGAGAATCGCTTGGACCTGGGA | |
| | | | | | GGTGGAGGTTGCGGTGAGCTGAGATCACGCCATTG | |
| | | | | | CACTCCAGCCTGGGCAACAAGCGAAACTCTGTCTC | |
| | | | | | AAAAAAAAAAGAAAAGACAGTAGCTTATGTTCATG | |
| | | | | | TCAAGCACCTCTCATCACAGTCTAGTTCCAAGGAAA | |
| | | | | | AAATTCCCAGCGTTTTTACATTCGGTGCTGCGTCA | |
| | | | | | TCTGAAATCGGCACATTCCATGGAGGAAGGAGTCC | |
| | | | | | TGCTTTGTGCATGTATCCTAGGGTTTAATGTTGGT | |
| | | | | | AAATGAGTCACTCTAGCATTGTAGAAGGCTCCCTG | |
| | | | | | AGACTCCTGCAGCAGTCGACCAAGCCCAAGGACAT | |
| | | | | | AATTGAATCTGGAGAGTCCTGGGGCCTGTTTTGAA | |
| | | | | | AAAGACTTGAAATACACATAGGAAGAAAGGCATAA | |
| | | | | | AAATAAATTGCACTTGTCTCTGCTGTGAGTATGTG | |
| | | | | | TTCCAACTTTTCAGTGATGGCTTTGAGAATTCTTCAA | |
| | | | | | ACTTGACTGGCTCTAAGTGTATCTGGTGGCTTTTGT | |
| | | | | | ATCGTAACCTGAAACTGGCTTAGTACTTTTTCCTAA | |
| | | | | | AAGCTCAGGATTTGAGAATGAGGACCCCTTCGCCA | |
| | | | | | GGAAAACATGTATACACTCAAAATTTTGCTTGCAGT | |
| | | | | | TCTAGGGTGTTTAGACCTTTCTCAGATACCTGTGCA | |
| | | | | | TCTTATGGGTTTGTTTTTCTCTTGAGACAGTCTCA | |
| | | | | | CCCTGTGCCCAGGCTGGAGTGCAGTGGCATGGTCT | |
| | | | | | CAGCTCATTTGCAGCCTCCGCCTCCTGGGTTCAGGTG | |
| | | | | | GTTCTGCCTCAGCCCCTTGATCGGCTGGGATTGCAT | |
| | | | | | GCATGTGCCACCATGCCCGGCTGATTTTTGTATTTTT | |
| | | | | | AGTGGAGATGGAGACAGAGTTTCACCATGTTGG | |
| 11 | | Ac010791 | 11 | | ACATTTCAGTTGGGAACAGATTGCTCCATGGTAATG | |
| | | | | | TGATCACTATGTACCCAACAATGGCTCTTTCTTCCT | |
| | | | | | AGCGTCAATGCAGATGTTATTTTCACCTTAACTGTT | |
| | | | | | ATCATTGTTGTTTCTAACCACATGAAAGTGTATCCT | |
| | | | | | TTATATATCTGAAGTAAATTCATACTAGTGGTGTAA | |
| | | | | | CATCTCCAGCCATTTAAGTGTAAAAACAGAAAACG | |
| | | | | | TATGATGTGTTTACGTACTGTTTTATACTCCTAACGC | |
| | | | | | ATGAAGAGAAGATCCTTTTATTCATTGCCTATACTT | |
| | | | | | TTATTTCTAAACTTTCTGTAACACTTTATCTTATATC | |
| | | | | | CAGCATAGAATTAAGATTTGCTTTTTCGATTTAATCT | |
| | | | | | GACAATATTTTTTCCTCTAATAAGAGTCAAGTCCAC | |
| | | | | | TTACTTTTAATGATAAGTTGTGTTTGGTTATATTTTG | |
| | | | | | ATTACAGTATATTATGCTATGATTTATATGCACATA | |
| | | | | | TCTGTCTTTTGCTGTCTTGTTTGTTTTTATTGCTTTTG | |
| | | | | | TTTTGATGTTGTGATATTTGGAAGAGTTAAACTTTT | |
| | | | | | ATTCTGATGGCTACCTTATGTAATTTCATAAAATCA | |
| | | | | | TCTCTTTTCTTTTGGACAGTAGCTAATGTCTCTAAACT | |
| | | | | | AAGAACAATGGTATTAGCTGTATTCTCTTTCTTGTC | |
| | | | | | CTCCCTATGTGATTTTTTCATCCCACAATTTTGATTTAA | |
| | | | | | TCATATTAACTTTGTTTCCCCTGGTGCCATTAAGTAT | |
| | | | | | GCTTACATTTCTATAAACAATATCCTTTG | |
| 11 | | Ac010791 | 281 | | GAAAGGGCCAAATACGACTCTTAATGATACAACAG | |
| | | | | | CTAAATATAGGTCTGATGCTCATTCCGTGTGGACAA | |
| | | | | | CAATAGCAGCCATTCCCACAAATGGCTGATTTGTAG | |
| | | | | | GAAGTAAACACTACTTTTGCAGAATCTTACATGATT | |
| | | | | | TCAGTAGAAGGGCAAGGACATTTCAGTTGGGAACA | |
| | | | | | GATTGCTCCATGGTAATGTGATCACTGTGTACCCAA | |
| | | | | | CAATGGCTCTTTCTTCCTAGCGTCAATGCAGATGTT | |
| | | | | | ATTTTCACCTTAACTGTTATCATTGTTGTTTCTAACC | |
| | | | | | ACATGAAAGTGTATCCTTTATATATCTGAAGTAAAT | |
| | | | | | TCATACTAGTGGTGTAACATCTCCAGCCATTTAAGT | |
| | | | | | GTAAAAACAGAAAACGTATGATGTGTTTACGTACT | |
| | | | | | GTTTTATACTCCTAACGCATGAAGAGAAGATCCTTT | |
| | | | | | TATTCATTGCCTATACTTTTATTTCTAAACTTTCTGT | |
| | | | | | AACACTTTATCTTATATCCAGCATAGAATTAAGATT | |
| | | | | | TGCTTTTCGATTTAATCTGACAATATTTTTTCCTCTA | |
| | | | | | ATAAGAGTCAAGTCCTTACTACTTTTAATGATAAGTT | |
| | | | | | GTGTTTGGTTATATTTTGATTACAGTATATTATGCTA | |
| | | | | | TGATTTTATATGCACATATCTGTCTTTTGCTGTCTTGT | |
| | | | | | TTGTTTTTATTGCTTTTGTTTTGATGTTGTGATATTT | |
| | | | | | GGAAGAGTTAAACTTTTATTCTGATGGCTACCTTAT | |
| | | | | | GTAATTTCATAAAATCATCTCTTTCTTTGGACAGTA | |
| | | | | | GCTAATGTCTCTAAACTAAGAACAATGGTATTAGCT | |
| | | | | | GTATTCTCTTTCTTGTCCTCCCTATGTGATTTTTCAT | |
| | | | | | CCCACAATTTGATTTAATCATATTAACTTTGTTTCCC | |
| | | | | | CTGGTGCCATTAAGTATGCTTACATTTCTATAAACA | |
| | | | | | ATATCCTTTGACTCCCAGGCATTTACAGATGAGCAGT | |
| | | | | | CAGTAAAATCATTCTGAGGAATACTTTCTCTTTCCT | |
| | | | | | TTTCTTCCATTTTTTCTTAGTTGTATCATTTCTCTGAT | |
| | | | | | GGGTCTATTTCTTTAAAACAAAGGGAGGGGAGTCT | |
| | | | | | CTCATTTACATTAGTTTTTTTCATAGCCTTTTGGACT | |
| | | | | | TTGCAATTTCTATGTTTTGGAACCTATTTCTTACAGT | |
| | | | | | TTTTCTATGCTAAACTCTGTCCTGGTCAGTTCCAGA | |
| | | | | | GTGTATGAAGAACCAAATCATGTAATTGTATGTGAC | |
| | | | | | CTGGCTGTAGTGGAACAAATTTGACTCTTAAGTATG | |
| | | | | | CAGGCTCTAATTTTCCTGTCTGGTTTTGGTAAGTATT | |
| | | | | | CCTTACATAGGTTTTTTTCTTTGAAAATCTGGGATTG | |
| | | | | | AGAGGTTGATGAATGAAAATTAAACCTTCACTTTTG | |
| | | | | | TTGTATATAGGTTTGCAATATTTAGGTCAGAGTGGA | |
| | | | | | GTTTTAAGGTCATGAAGGGGGCTGATGACTTACAA | |
| | | | | | ATAATGGGCTCTGATTGGGCAACTACTCATCTGAGT | |
| | | | | | TCCTTCCATTTGACCTAATTAAGCTTGTGAAATTTA | |
| | | | | | CACTAAGCCATGAGCTCATCTTTAAAAAGTTTTGTT | |
| | | | | | AAAAGATTTTCAGCTGTTCCAAATGGGACTTATTAG | |
| | | | | | TGGAATGTGTTTTAAAGGATCATATCAGATGAATGA | |
| | | | | | AAGGTATTTGATCCTTTCTTTCCTTAATAATAAAAT | |
| | | | | | GATGGTTTGGAAAAATAGGCTACAGTCTAACCACA | |
| | | | | | GTGCTATTATTAGGCTTTCTTGTTAAACATAGGTCT | |
| | | | | | AAGCCTAAGTATGTCAATACAACAAATACTTACTGT | |
| | | | | | TTCATTTCTAGTAATAAAAAAAAAAGTCTTTCTGGC | |
| | | | | | TTTGTAAAATAAATTTACATCTATAAAGAACATTTT | |
| | | | | | TATTCGTAAGGAGGGGTATGTCTCTGTGCACTGGAA | |
| | | | | | GAGAGGGAGGACTAAATCACTGGGAAGTCTTATGA | |
| | | | | | TAAAGAAGCCATTGGCTTAAATCAGCAAAGCAAGC | |
| | | | | | CATCCCTTGGTTTTAAGGTGTTTTTCCTGGCCATCCTG | |
| | | | | | TCTTGACTAGAACTTTACCTACACCTTCCTTTTTGGT | |
| | | | | | TTAGGCAAATTATAGTATCTAAACCTGAAGTCTCAG | |
| | | | | | CTCTGTGTCTTTTGAGATATAAATGTTCTACCATGTCT | |
| | | | | | TCTCTGGAACCTGATAACTATCTATCTCTTTAAAAT | |
| | | | | | GGAAGTCTAGGGAGATGACTCATCAGAAAGTCTAG | |
| | | | | | GAAGATGACTCATCAG | |
| 12 | | A1008730 | 12 | | ATAGTCCCATTTTATGGATGTACATCTTAGTATTCA | |
| | | | | | CGTAGACTCAAGATGATTTTTATGCAGATTTCTGGA | |
| | | | | | GCTCTGTCTCTTGACAGCTTTCTCTTTCTCTTGTGGTG | |
| | | | | | CTCTCTTTCTCAAATTGTGGTTGCCCTCCTAAGTTCC | |
| | | | | | TGTCTCTCTCTTAAGCCCAGCAAAACCACTGTACTC | |
| | | | | | TGCTAGGTTCTCCTTCTTTGTATATCAGTCGATAGA | |
| | | | | | GTGCCTCCAGGCAGAAGGCTGGAACTCAGTTCATTT | |
| | | | | | TTCTTTTCCCAAGGGATCACAGTCCTCCTGTACTACC | |
| | | | | | TGTTGTTCAGATTTTCAAAGCGGTTACTTTATATATT | |
| | | | | | TTGTCTACTTTTACTATTTTTTATAGCAGATGCTAGT | |
| | | | | | CCCATATTAGTTACTCCATCATTGATTC | |
| 13 | | Ac068709 | 13 | | CGGGAGACTAGAGATGAGCTGACGCAGGAAAATAA | |
| | | | | | GGCAACTTCCACACCAGGAAGAATCAAAAGAGGGC | |
| | | | | | GAGCAGAAAATGTGCAAAGATCACCCAGGCTTTGC | |
| | | | | | TTCCCACACGAGCAATTACAATGCTCCTTGCGGAA | |
| | | | | | TTCTCAACCACACCAGAAGACCAACAGATCAATTT | |
| | | | | | GAGTTACTCTTTTTAAGGAAAAAGTGACCTACATTT | |
| | | | | | CATGAAGCAAAGAGATACAGCCACACACAGGAGCC | |
| | | | | | GTTTGTTTTAATTAGATTGCTGGTTTCCCTGGCCAG | |
| | | | | | GACCCAAAACCACTGTGTTTCCCCATAGATACAATT | |
| | | | | | GACAAATAAAATACATGACACTCATGTGAATCAGA | |
| | | | | | ATTTC | |
| 14 | | Ac023602 | 14 | | GATATTATTAATTTCTTAAAACTGAATCCTCCATAGA | |
| | | | | | ATCCTAAAATTTGTCATGGACTATAACATATATCAC | |
| | | | | | ATTTAATTTTCTCAAAGGTCTTGTAGGGTACATAAA | |
| | | | | | GGAGGGACTGCCCCTGATTTTACATTAAATTGCA | |
| | | | | | TTAGGTGAGAGAATTTTTGTGGGACCAGAGGAAGA | |
| | | | | | AATGCGTTATATGTCTCAGTGCTCTTGGCATAATTG | |
| | | | | | TGTATGCAGAGTACATCTTATTTTGGTGATGTTTTTG | |
| | | | | | TATGAAAGACTTTTGAGCTCATTGTTATGACTCAGC | |
| | | | | | AAAACTATGGGGTTATAGTTAATCTGACTCATTC | |
| | | | | | CTTAATGGACATAATTATTTTACAAGGGTAAATACT | |
| | | | | | GTTTCTCCATCAAGACTGGTAAACTATTCCATGTA | |
| | | | | | TAAAGGTCAGCTACATCAGTTTTGGTTAGAGGTGTG | |
| | | | | | GACATTTAAAATAGGTGGATTAAAATAAAGAATAT | |
| | | | | | TCCAAAGATAATTGCCCAAAATATCCAAACCAGTA | |
| | | | | | TTTGCAGCTCAAGTGTATACCTGCCGTGATGGTTAT | |
| | | | | | CTGAACATCATTTTGTACCTTTGTTTGCATTTATTTA | |
| | | | | | TGTTTTATTTTATATTAAACATATGCAGCCCATGTA | |
| | | | | | AGTTTCAAAACAGTTAATAATTCTATCTTCTC | |
| 14 | | Ac023602 | 266 | | GATATTATTAATTCTTAAAACTGAATCCTCCATAGA | |
| | | | | | ATCCTAAAATTTGTCATGGACTATAACATATATCAC | |
| | | | | | ATTTAATTTTCTCAAAGGTCTTGTAGGGTACATAAA | |
| | | | | | GGAGGGACTGCCCCTGATTTTACATTAAATTGCTTA | |
| | | | | | TTAGGTGAGAGAATTTTTGTGGGACCAGAGGAAGA | |
| | | | | | AATGCGTATATGTCTCAGTGCTCTTGGCATAATTG | |
| | | | | | TGTATGCAGAGTACATCTTATTTTGGTGATGTTTTTG | |
| | | | | | TATGAAAGACTTTTGAGCTCATTGTTATGACTCAGC | |
| | | | | | AAAACTATGGGTTGTATTAGTTAATCTGACTCATTC | |
| | | | | | CTTAATGGACATAATTATTTTACAAGGGTAAATACT | |
| | | | | | GTTTCTCCATCAAGACTGGTTTAAACTATTCCATGTA | |
| | | | | | TAAAGGTCAGCTACATCAGTTTTGGTTAGAGGTGTG | |
| | | | | | GACATTTAAAATAGGTGGATTAAAATAAAGAATAT | |
| | | | | | TCCAAAGATAATTGCCCAAAATATCCAAACCAGTA | |
| | | | | | TTTGCAGCTCAAGTGTATACCTGCCGTGATGGTTAT | |
| | | | | | CTGAACATCATTTTGTACCTTTGTTTGCATTTATTTA | |
| | | | | | TGTTTTATTTTATATTAAACATATGCAGCCCATGTA | |
| | | | | | AGTTTCAAAACAGTTAATAATTCTATCTTCTCAATG | |
| | | | | | AAAAAAAAATCTGATTCCTAGAGCTCTACCCTTTCA | |
| | | | | | TTTTTACTCATATGGCTCTCTCTTATGAAGGATTT | |
| | | | | | TCTGTAATCAAATATTTACGTGAGACTTGTATAAAA | |
| | | | | | ATTTATTCTTCGTAGACAAAAAATATAGATATTGGTA | |
| | | | | | GAATATGGCCAAGGAAATGTTATTTTGAATGTAATC | |
| | | | | | CTGAAACATCTGAATATGCTTGTGTTTAAATGTATT | |
| | | | | | ATTATTTTAATTTTTAGGAAAAGCCCGATGGCTCCC | |
| | | | | | CAGTATTTATTGCCTTCAGATCCTCTACAAAGAAAA | |
| | | | | | GTGTGCAGTACGACGATGTACCAGAATACAAAGAC | |
| | | | | | AGATTGAACCTCTCAGAAAACTACACTTTGTCTATC | |
| | | | | | AGTAATGCAAGGATCAGTGATGAAAAGAGATTTGT | |
| | | | | | GTGCATGCTAGTAACTGAGGACAACGTGTTTGAGG | |
| | | | | | CACCTACAATAGTCAAGGTGTTCAAGCAACCATCTA | |
| | | | | | AACCTGAAATTGTAAGCAAAGCACTGTTTCTCGAA | |
| | | | | | ACAGAGCAGCTAAAAAAGTTGGGTGACTGCATTTC | |
| | | | | | AGAAGACAGTTATCCAGATGGCAATATCACATGGT | |
| | | | | | ACAGGAATGGAAAAGTGCTACATCCCCTTGAAGGA | |
| | | | | | GCGGTGGTCATAATTTTTAAAAAGGAAATGGACCC | |
| | | | | | AGTGACTCAGCTCTATACCATGACTTCCACCCTGGA | |
| | | | | | GTACNAGACAACCAAGGCTGACATACAAATGCCAT | |
| | | | | | TCACCTGCTCGGTGACATATTATGGACCATCTGGCC | |
| | | | | | AGAAAACAATTCATTCT | |
| 15 | | Ac011244 | 15 | | GTAGTAGACATTTTTCCATCTCTTACCTTTATAAAGT | |
| | | | | | AAATATATATAAGAATGAAGAATTAAACTAATAGA | |
| | | | | | ATTGTCGAATTTTATTTCATTTATAATATAAGTAAG | |
| | | | | | CAAATAGACCGAGACAGGTTGGTTACACACTTAGT | |
| | | | | | GACAGAACTAAGACTCCATCCTACAATCTTCTGTTA | |
| | | | | | TAGCCACAGGTAAAATAATAACTGCCATCCT | |
| 16 | | Ac026974 | 16 | | TTGTTTTTTGATCATTTGCATCTTCATTATAAAGGAA | |
| | | | | | GTCCAGAGAATGTATGGCTATGTCACATTTTGGGCA | |
| | | | | | ATCTCTCTGGGCTAACTTTCTTTAAAAGGTCAGATT | |
| | | | | | CTCCTGGCAACAGAGAGAGACTCCGTCTC | |
| 17 | | Ac026843 | 17 | | CAAATTAATTTAAAAAGTAAACAGAGACAGGGTTT | |
| | | | | | GCTGTCGCCCAGGCTGGAGTGCAGTGGCGAGATCA | |
| | | | | | TAGCTCGTTCCAGCCTCAAACTCCTCGGCCCAAGAG | |
| | | | | | ATCTTTCCACCGTGGCCTCTCAAAGGCTTGGGATTA | |
| | | | | | CAGGGGTGAGCCACCCCACCCAGGCCCTGTTATTCC | |
| | | | | | ATACATTTTCCATAAAATTATTTTATAATTTTTGTTT | |
| | | | | | TGTTTTGTTTTTATTTTATAAATTGTGTGTGTGTGT | |
| | | | | | CTCGCTTTGTTGCCCAGGCTGGAGTGCAGTGACGCG | |
| | | | | | ATCTTGGCTCGCTGCAACCTCCACCTCCCAGGTTCA | |
| | | | | | AGTGATCAGCTCTTGCCTCAGCCTCTGGAGTAGTTG | |
| | | | | | GGACTACAGAGACATGCCCCACCGCACCGGCTAAT | |
| | | | | | TTTGTATTTTTAGTAGAGGCGGGGTTTCACCATAT | |
| | | | | | TGGCCAGGCTGGTCTCGAACCCCTGACTTCAAGAG | |
| | | | | | ATCCATCCGCCTCGGCCTCCCAAAGTGCTGGGATTA | |
| | | | | | CAGGCGTAGCTGCCGCGCCGGCCAAAATTATTCCA | |
| | | | | | TAAATTTATCCATAAAAATTCCACATAAATTTTCTG | |
| | | | | | GAGTTTGATTATGTATTAGGCTTGTTGGGAAATTTA | |
| | | | | | TACCCTTGTGAAGAATT | |
| 18 | | A1096868 | 18 | | ACGGGTTGATGGGTGCAACAAACCACCATGGCACA | |
| | | | | | TGTGTGTAACACATCTATGTAACAAACCTACATGTT | |
| | | | | | CTGCACATGTATCCCAGAACTTAAAGCATAATTTTT | |
| | | | | | AGAAAAGTATTCAGCTGAATGTGAATACAGTCATG | |
| | | | | | TGATCTATGTCAATCCTATGGCTTTGTTAACCTGCA | |
| | | | | | GCAAATTCACAATCACAGAACAATTAATTGATCAG | |
| | | | | | ATTTAGGCAAAGTAACTGCCTCTTAATTATTTTGGA | |
| | | | | | GGCCAATAACATCTTTTGACAGAGCATGGTGGCTCA | |
| | | | | | CACCTGTAATCCCAGCACTTTGGGAGGCCGAGGCA | |
| | | | | | GGCAGATCACGAGGTCAGGAGTTTGAGACCAGCCT | |
| | | | | | GGCCAATATGGTGAAACCCCATCTCTACTAAAAAG | |
| | | | | | ACAAAAATTAGCC | |
| 19 | | A1136528 | 19 | | GGCCTCCAGAACCAAGAGAAGACAGGGGAGTAGG | |
| | | | | | GATTCTCCCAGGGCCCCCCAAAGACAGGAAGAGGG | |
| | | | | | GGAAATGTATTCTCCCGGGGTCTCCAGAAGCAGCC | |
| | | | | | AGCCCTGCCCGCAGTTTGGCTTTAGCTCCCTGGTAC | |
| | | | | | CCATCTCGGACTCTGACCTACAGAACTGTAAGAGA | |
| | | | | | GTAAATTTATCTCATTCTGTGCTGCTCATTGTGTGGT | |
| | | | | | CATTGGTTACGGCAGCCACAGAAAACAGACAGTGC | |
| | | | | | GCACATCCGCATGGTCCCCTCTCCAGCTCTTGCCTG | |
| | | | | | ATAGGCATAAACGAGGGCAGCTGGGCGCGGTGGCT | |
| | | | | | CACGCTTGCAATCCCAGCACTTTGGGAGGCCGAGG | |
| | | | | | CGGGTGGATCATGAGGTCAGAAGATTGAAACTATC | |
| | | | | | CTGGCCCACATGGTGAAACCCCGTTTCTACTAAAAA | |
| | | | | | TACAAAAAATTAGCCAGGCGTGGTGGCACGTGCCT | |
| | | | | | GTAGTCCCAGCTATTCAGGAGGCTGAGGCATGAGA | |
| | | | | | ATCGCTTGAACCTGGGAGGCAAAGGTTGCAGTGAG | |
| | | | | | CCAAGATGGAGCCACTGCACTCCAGCCTGGGCGAC | |
| | | | | | AGAGAGAGATTCTGTCTC | |
| 20 | | Ac011236 | 20 | | GAAGTGCAGTGGTGTGATCACAGCTCATTGCAACCT | |
| | | | | | TGAACTCCTGGGCTCAAGTGATCCTCCTGCCTCAGC | |
| | | | | | CTCCCGAGTAAGTGGGATACAGGCATGCACTACCA | |
| | | | | | TCCTTGGCTAATTTTTTTTAAATTTTTTGTAGAGAA | |
| | | | | | TTTTTGTTTCTCTACCAAGTTTTTGTTGCCCAGGCTG | |
| | | | | | GTCTTGAACTCATGGCCTCAAGCAATCCTCCCACCT | |
| | | | | | CAGCCTCATAAAGCACCAGGATTACAGGCATAAGC | |
| | | | | | CACTGTGCCCGCTCTGTCTTATCTAACTGGGTAATC | |
| | | | | | ACTCAATAAAATTAAGTTCTTATTTTTTC | |
| 21 | | Ac008576 | 21 | | TCCCCATGAGAAGTGATGGTGGCCTCGACTGGGAG | |
| | | | | | TCGGGAGTCATGGATCCAGCTCACATTTTCGTTGAG | |
| | | | | | GAGGAAGGGTGGAGGTGGATGAAAAGAGGAGGCA | |
| | | | | | GGTCTCATATTCCAGGAAGGCAAGAATTAAAAAAA | |
| | | | | | AAAAGGAATGAAATGAAATGAAAAGAGGAGGCAG | |
| | | | | | GGTGGTGTCTAGGTTTACAGCTTAGGGACTTGCGTG | |
| | | | | | AATTAGGGTATCTTCTACTGTAGTAGGAAGACTAGG | |
| | | | | | GGAGGAACAGGTCTTGGGGAGTT | |
| 22 | | A1136163 | 22 | | ATACTGGACTTCTTCCACGACTCTGTTTACTTCATCT | |
| | | | | | TATGTAAAGTGCAGATTTACTGCGCACAAGGCATA | |
| | | | | | CATGATTGAGGGTTCCTCTACCCTCTCCTTTGCACA | |
| | | | | | TGCAACATTTGGATTCAGTGCACACTAATCAAAGAC | |
| | | | | | TCACAAGAAAGTAACCGTTTGTCTCATTTTTTCTAC | |
| | | | | | CCTCCTCTTTTCTCCTTCCTCTCCAGCCCACTTTTCC | |
| | | | | | CCCTTTAAATACTGAAGCCCTCAAAACCCTCTTTGG | |
| | | | | | AAAAAGTGCAGGACACAGATCCTACTGTGGCTTGT | |
| | | | | | GTCTCTTTTTCCCTCCCTAACCAGATGCATCCTCAAC | |
| | | | | | CTTAGCAAAATAAACCTCTAAATTGATTG | |
| 23 | | Ac026314 | 23 | | ATTCCTAGAAAAATACAAACTACCAAAACTGACTG | |
| | | | | | AAGAAGAAATAGATAGCATGAATAGAACTATAACA | |
| | | | | | GGAAATTGATCTAGTATTCAAAAACTATGCACAAG | |
| | | | | | CCAGGCACGGTGGCTCACACCTGTAATCCCAGCACT | |
| | | | | | TTAGGAGGCTGAGGCAGGTGGATTGCCTGAGCCCA | |
| | | | | | GAAGAGACCAGCCTGGGTAACATGGTGAAACCCTG | |
| | | | | | TCTATACAAAAATTAATTGAGTGTGGTGGCATACAC | |
| | | | | | CTGTAGTCCCAGCTACTCAGGAGGCTGAGGTAGGA | |
| | | | | | GGATCATTTGAGTCTGGGAGGTCGATGCTGCAGTG | |
| | | | | | AACTGTGATTACACCACTGCACTCCAGCCCGAGTGA | |
| | | | | | CAGAGCAGCACCCCA | |
| 24 | | A1354926 | 24 | | TCCAGAGTTCTAGAACAAGTAGATCTAGAACAATT | |
| | | | | | GGATATCCAAATGCAAAAATCCCAGACATATACCT | |
| | | | | | CCAAGCTTATATAAAAATTATTTAAAATGGATTAT | |
| | | | | | AGAACTAAGTAACTGTAAAATGTGAAACTTACAAA | |
| | | | | | AGAAAACAGAATATCTGCACGACCTTGGGTTTGGT | |
| | | | | | GTGTTCCCTGAAAGAAAACAGTGATAAATTAGACT | |
| | | | | | TTACCAAATTAAAAATTTTGCTCTGTAAAAGACAG | |
| | | | | | CTTTAAGAGAACAAGATAAGCCACAGACTGGAAGA | |
| | | | | | AAATATTTGCAAATCATAAATTTCATAAAAGATGTG | |
| | | | | | AATCCAAAAGATATAAAGAACTCTCAAAACTCAGT | |
| | | | | | AATTAGAAAACAGTTTTTTAAACGGGCAAAACATTT | |
| | | | | | GAGTAGACAGTTCACCAAAGAAAAAGTGTGAATGG | |
| | | | | | TAAATATAAGCACATGAAAAAATATAGCTCATTAG | |
| 25 | | Ac004168 | 25 | | GCACCATGTAATAATAGATAAAATATTAACTGTTAT | |
| | | | | | AAGTTAATATTGTATACATTTATGTATTAAGCAAAG | |
| | | | | | TATACATCTCAATTCCAAACATAATTTTCAGAGTGA | |
| | | | | | AAACGATACAGTAACTAGTAAAACAATATGCCGAG | |
| | | | | | AATCGT | |
| 26 | | Ac068619 | 26 | | GGAATGCTATCATTTTAAATTATTTTGGAGCTCATT | |
| | | | | | AAAGTAAGTCTGCACTGGCCAACTTTTTATTTATTA | |
| | | | | | ATTAAATTTTTGCCTAGC | |
| 27 | | Ap002338 | 27 | | ATGCCCTTGACCTAAGGCCTCTCCTTTCTTTTCCTC | |
| | | | | | TCTGGGGTGCTGCCTCATCCTTCTGGTCTTCAAAAC | |
| | | | | | CGTTTCCCTGGGAAAACATCTTTGACTCAGCAGGCA | |
| | | | | | GGGATCATGCCCCTGCTGTGTCTGTGCATAACTTTC | |
| | | | | | TGTGGCTACTCTGTCTTGGTCTGTGATGTACTTTAT | |
| | | | | | AATAATTTTGGTCTTTCCTCCAGTGTCACAATACTG | |
| | | | | | GAAGTCTGTTTCTTTTTCTCTGTGTTGTATCCTTAGT | |
| | | | | | GCCTGAAAGGTAGGAGGTTCTCAATAAATATTTGTT | |
| | | | | | AAATAATCAAGTAAATGGAGTCTGGTGGAAAAGAG | |
| | | | | | AAAAAATAAGTGTAGAATGTGTGTGCAAGAAAGGA | |
| | | | | | GGGGTAGGGGGATGAAAAAGATAACAAAAGCACA | |
| | | | | | TAACAAAACAACA | |
| 27 | | Ap002338 | 267 | | TTTCACGATTAGTTTTAGCTTAAAAATGTCAGCTCT | |
| | | | | | GGGCTTAATGAAGAAAATATGGATATACTTTATGTC | |
| | | | | | AATGCATTAAAGTGAATGGCCATAAAAGCTTATCC | |
| | | | | | CAGAGACAAAACAATTCAGATATAAGAGAAGTGGG | |
| | | | | | AGAGTGGAAGGTTTATCTAATCTTCTGTAGGCAACT | |
| | | | | | CCACAGCTACAACCAGAAGGCCATTTTGTTACAGG | |
| | | | | | CCTGAAAGCCCCGTTTTCTTTTTATTCTTCTTTGAAA | |
| | | | | | CCTTTAGAAGGAACAAAGTATTGGCTACTTTTTACC | |
| | | | | | GCTGATGTCAGTGTAAGAATCTTGTGATAACATAG | |
| | | | | | ATTTACTCTCCCTGCTGAAAATCACTATGTGGCTCA | |
| | | | | | TCAGTAACACAACTAGACATGATGACTTAATGCAA | |
| | | | | | AGGAAGTCCTATGTAAATGAGCAATGAAATTGCAA | |
| | | | | | CTGTGTATAAGGAACAAAATAGAATATGAAACTCC | |
| | | | | | AGAATCTTTTGTTTTCATTTCTGTTTCTCCCAAGGCT | |
| | | | | | CTATCATTTCAAAACTCCAGAATCTTTCAGCATGCAA | |
| | | | | | TTGTCTCCTGATATCAGCCCCTCTCTTGTTTTGTTTT | |
| | | | | | CTTTTTTTTTTTTTTAATCACAGTGAGCCACAACCTA | |
| | | | | | GGAGTCTTTTAGTGGTTTCTACTTGGTTTGCTCTGCA | |
| | | | | | GCCTACCAGCAGATTTCCTACATTCCGGTCTTGTTC | |
| | | | | | CCCTCTAGCCCATTCTCCACACTGCAGTCATAATGA | |
| | | | | | AATTTCTTTCTTTTTTGGGGGGGATGGAGTCTCACT | |
| | | | | | CTGTCACCCAGGTTGGAGTGCAGTGGCATGATCTCG | |
| | | | | | GCTCACTGCCACCTTTGCCTCCTGGGTTCAAGCGAT | |
| | | | | | TCTCATGCCTCAGCCTCCCAAGTAGCTGAGATTATA | |
| | | | | | CGCACCTGCTACCACGCCCAGCTAATCTTGTATTTT | |
| | | | | | TAGCAGAGACAGGGTTTTGCCACGTTGGCCAGGCT | |
| | | | | | GGTCTCTAACTCCTGACCTCAAGTGATCGCCCACCT | |
| | | | | | TGGCTTTCTCTCTCTTTTTTTTTTTTTGGATTTTGAGA | |
| | | | | | CAGGATCTGGCCTCGTTGCCTAGGCTGGAGTGCAGT | |
| | | | | | GGCACGATATCAGCTCACTGCAACCTCTGCCTCCTG | |
| | | | | | AGCTCAAGCCATCCTCCCACCTCAGCCTCCTGAGCA | |
| | | | | | GCTGGGACTGCAGGTGTACACCACCACGCCTGGCT | |
| | | | | | AATTTTTGTATTTTATTTTATTTATTTTTTTTGGTAGA | |
| | | | | | GACGGGGTTTTGCTGTGTTGCCCAAGCTTGTCTTGA | |
| | | | | | ACTCCTGGGGCTCAAGCGATCTACCCATCTGGCCT | |
| | | | | | CCCAAGGTGCTGGGATGACAGGCATGAGCCACCAC | |
| | | | | | AGCTGGCCTATAATGAAATTTCCAACTTACAGCTAT | |
| | | | | | TGCCATTATCCAAAGCCCAGAATCCCTGATTTCCTT | |
| | | | | | CCATAGCCCTTCATGGCCTGACCAGTGCCTGACTCT | |
| | | | | | CCAGCCTCACACTTCATATTCTCTCTGTACTGCTCTG | |
| | | | | | CACTGTAGCCTCATTGAGTTGCTTTCACGTCTTTAA | |
| | | | | | GTGTTGTGTTCTATTTTTTGTGGAATTCAGCATATGT | |
| | | | | | TATGCCCTTGACCTAAGGCCTCTCCTTTCTTTTCCTT | |
| | | | | | CTCTGGGGTGCTGCCTCATCCTTCTGGTCTTCAAAA | |
| | | | | | CCGTTTCCCTGGGAAAACATCTTTGACTCAGCAGGC | |
| | | | | | AGGGATCATGCCCCTGCTGTGTCTGTGCATAACTTT | |
| | | | | | CTGTGGCTACTTCTGTCTTGGTCTGTGATGTACTTTA | |
| | | | | | TAATAATTTTGGTCTTTCCTCCAGTGTCACAATACT | |
| | | | | | GGAAGTCTGTTTCTTTTTCTCTGTGTTGTATCCTTAG | |
| | | | | | TGCCTGAAAGGTAGGAGGTTCTCAATAAATATTTGT | |
| | | | | | TAAATAATCAAGTAAATGGAGTCTGGTGGAAAAGA | |
| | | | | | GAAAAAATAAGTGTAGAATGTGTGTGCAAGAAAGG | |
| | | | | | AGGGGTAGGGGGATGAAAAAGATAACAAAAGCAC | |
| | | | | | ATAACAAAACAACAAAA | |
| 28 | | A1158062 | 28 | | TTGCAAATATGTTTTGAAATATATTTTTGGCTTTTGA | |
| | | | | | ATTCCCTTGAGAAGTGTAGAGAAGAATATACA | |
| | | | | | AATCAAAGAGGATTTAATATATTATTCATGCATAT | |
| | | | | | CTTTCCTCTGAGATTTTGTTTTGTTTTAAATCTTGG | |
| | | | | | AAAGTATGTTACTCATTTCAGTATTTCCACTGACTTT | |
| | | | | | CACTGGTAGATGGTTCTTACTAAATTAATTTCCTTGC | |
| | | | | | CATACTATGTTAAAAATTTTATTCTCAATAGATATT | |
| | | | | | AGCCCCATATTGTTTTAACCACCATTGCTTTATGTTA | |
| | | | | | CTAATCTTTTTGATGGTCCTGGAAAGAACTGATTTT | |
| | | | | | AATTTCTATTTATTAATGAATTTTTGTTTTTACAGTT | |
| | | | | | TfAACTCATGTTACCTAATCATAGCATAAGAGGACT | |
| | | | | | GTTGCACAGTGCTCCTGCATAGAGTACAGCAACAG | |
| | | | | | TGGCTCCATGCATGTTACCTGCTGATGGGATGGATG | |
| | | | | | CTAGCTGAGTGTTTGAGTAGACTAATCATGATAGAT | |
| | | | | | ATATTTCCTGTTGTGTGCCAGACACTGTTTAGGAAC | |
| | | | | | TGATGATACAGAAATATGCCTTCAGGTACCTGACAC | |
| | | | | | CCTCGTGGGGAAGCAGACAGCCATCAATTGTGTGA | |
| | | | | | TGTAATGTGTCACTGTCACGAAAAAAAGAAGACTG | |
| | | | | | GGAAAGGGGACAGAGGATGAGGGAGTTGCTAGTTC | |
| | | | | | ATATGTCAGTCA | |
| 28 | | A1158062 | 268 | | TTGCAAATATGTTTTGAAATATATTTTTGGCTTTTTGA | |
| | | | | | ATTTTCCCTTGAGAATTGTGTAGAGAAGAATATACA | |
| | | | | | AATCAAAGAGGATTTAATATATTATTTCATGCATAT | |
| | | | | | CTTTCCTTCTGAGATTTTGTTTGTTTTAAATCTTTGG | |
| | | | | | AAAGTATGTTACTCATTTCAGTATTTCCACTGACTTT | |
| | | | | | CACTGGTAGATGGTTCTTACTAATTAATTTCCTGC | |
| | | | | | CATACTATGTTAAAAATTTTATTCTCAATAGATATT | |
| | | | | | AGCCCCATATTGTTTTTTGAGACAGGGTCTTGCTCT | |
| | | | | | ATTACCCATGCTGGAGTGCAGTAGTAGAATCAAAA | |
| | | | | | ATTTTTAGAGTCAGTATACTCATGTAAGCTAACATA | |
| | | | | | AATGAGAAAGAGAGAGAGCGAGAGAAAGAAAGGA | |
| | | | | | AAGGAGGAAGTGGGAAGGGGAAAAGAGGGGAGAG | |
| | | | | | GAGTGGAGGGAGGGGAGGGGAGGGGAGGGAGATA | |
| | | | | | CTCTTACTCAGAAATTTTCTTTCTTTGAAAATCCTT | |
| | | | | | ATGACATTTCTAAGAAGAAGCAAGAATAGTGTGAC | |
| | | | | | CTTTGCAAATTACCTTAAAGACAAAGAGGAGAAGA | |
| | | | | | AAGAGCCAAGCTAATACATGAAGAGGGAAAACAA | |
| | | | | | CCAGAAAAAATGACATTTCAGACACAATCATGGAC | |
| | | | | | AGAAATCCTACAAGTCAGTAGGGGCCACCTTTACCT | |
| | | | | | GCCAGGGGGACCACAAAAATAGGGGATTTCTGTCA | |
| | | | | | AGAAGGCAGGAATGTTCAGCAGAACACAGCTTCTG | |
| | | | | | AATCATCTGACTCTCTCAGAACCAAGACAAAACAG | |
| | | | | | TTTCAAATGCCTACAAGCCACAGGACCCAGGAAATA | |
| | | | | | CCGCAGAGTGGACACTTTCCCCCTCTACATAAAAGA | |
| | | | | | ACCTATTTCTTTTTCTATGCATCAGCTTCTCCAGTCCA | |
| | | | | | TCTTTCATTAAAAGGACTTGCCAATGGAATGAAAACT | |
| | | | | | CATATTTCAGGACTAAGATGGACAACAGGCCTCTC | |
| | | | | | CAGCTCTTCTCTGAAAAGTGAGCTTTTCGGTAGAGA | |
| | | | | | ACGAGCTTCCTTCACAAGAAGGGCACTCCCGCTGG | |
| | | | | | GTGTGAGCCAAACGCACATGCACGACACTTGCGCA | |
| | | | | | GCTAAGAATACGCACAGTGGGGAAAAGGCACAGA | |
| | | | | | AGCAGCCCCCGTCCTGCCCGAGTGCCACATCCCTTT | |
| | | | | | CTGGGCTTTTCATCCCCCACCCCCACCGCCTGCAAA | |
| | | | | | ATGAAAGAAAGATTGCAATAAACAAGGTGTAAGTC | |
| | | | | | TCAAACCTGCTCTTCACCTGGAGCTTGTAATCAGGT | |
| | | | | | GTCAGGCTCCCATCCACCCACAAGGAACAGAGAGA | |
| | | | | | TTTTGGTGTTGAAGCTTCAACCTGCCCTGCGAGCCA | |
| | | | | | ATCTTTATTTCAAAGTACTTTGTGCTGTAAGCTAAC | |
| | | | | | GGGAAAAAATGATCAAATGCCTCAAATCTCCCGTA | |
| | | | | | AGCAGGGACTGTGCCTGGGGGGAAAGGTGCTCACC | |
| | | | | | AAGGTGGGGGCACATCGGGTGTCTCCTGGTGCTTTC | |
| | | | | | TGCTGGCACTAACATTCTAAAACATGAAGCATTAA | |
| | | | | | GTACAGCAACATGGATCTTTTCCTTTTTAACATGGAA | |
| | | | | | AATACGTTTTCATAGAGCAGGAGGGAAAAGAACTC | |
| | | | | | TCTAAAAAACAGAGCTGAATAGGCTTAGCAAGAAA | |
| | | | | | AGAAATTCAGGAGATGGAGAGGAGGAGCTCTAAAA | |
| | | | | | CATCCACAAAAAAATAAACCATTTCATAGCAATGC | |
| | | | | | TGACCATTTTAATTGATTCTCGACGACAGAAGAACA | |
| | | | | | CAAGAAAAGGTAGATGATGTAATGCGATGGCTGCT | |
| | | | | | GAAGGCAAAAGTCACAAAACAAATTTAGCCCTTCG | |
| | | | | | AATACCACAGTAGCCACGGGTCAATATAAAAAGCT | |
| | | | | | TCAACGGTCAGGAGCAAAACTGGGGTGAAGGGGCT | |
| | | | | | ACTCCCCCATACATGTAATTTGTCCAAGCCCTGCCA | |
| | | | | | TAGCCACCACCTCCCTGGATCCTCAAAGCAACCCTA | |
| | | | | | TTATGCAAGACATGCTGATCCAGGTGCATCTGACGA | |
| | | | | | TTCAGAAAACCAGGACCAAGCCGTGGGGCACCGAG | |
| | | | | | CCTGAGCTAATAAGCGCAGAGTCGACCCTGGCAC | |
| | | | | | GAAGGTCTCCCAGCTCCATGAAGATGCATCATCAA | |
| | | | | | GAAGGTTGGGCCTCAAATTCTTTCCATTACACTTCA | |
| | | | | | TGTTTCTCCCTGGATTATCTCCATAAAGGAGAAAAA | |
| | | | | | CAATACCCAGAACACAATTCCAACTCTGAGAAATT | |
| | | | | | GTCTGATCTTCCTCCTTGTCTCTGCCCCTCAAAAAA | |
| | | | | | AATTTTAACCACCATTGCTTTATGTTACTAATCTTTT | |
| | | | | | TGATGGTCCTGGAAAGAACTGATTTTAATTTCTATT | |
| | | | | | TATTAATGAATTTTTGTTTTTACAGTTTTAACTCATG | |
| | | | | | TTACCTAATCATAGCATAAGAGGACTGTTGCACAGT | |
| | | | | | GCTCCTGCATAGAGTACAGCAACAGTGGCTCCATG | |
| | | | | | CATGTTACCTGCTGATGGGATGGATGCTAGCTGAGT | |
| | | | | | GTTTGAGTAGACTAATCATGATAGATATATTTCCTG | |
| | | | | | TTGTGTGCCAGACACTGTTAGGAACTGATGATACA | |
| | | | | | GAAATATGCCTTCAGGTACCTGACACCCATCGTGGGG | |
| | | | | | AAGCAGACAGCCATCAATGTGTGATGTAATGTGTC | |
| | | | | | ACTGTCACGAAAAAAAGAAGACTGGGAAAGGGGA | |
| | | | | | CAGAGGATGAGGGAGTGCTAGTCATATGTCAGT | |
| | | | | | CA | |
| 29 | | A1132777 | 29 | | AGATAACAACAGAGATATTTTTTTCATTTTAACCTG | |
| | | | | | AAGGAATGCAGTTAATATGGTTATAGAAACAGGTA | |
| | | | | | GATTGATGGCATTGGTGTTTAGAAATGAGATTATTT | |
| | | | | | TGTCTCTATAGTATGAGGCTAGGTCACTAGCTATG | |
| | | | | | ATTGAGGTGAGAATGGGAAATGTGAGAAGTCTGAG | |
| | | | | | G | |
| 30 | | AC008762 | 30 | | TAATTTGTGGATAGCTATGGCAAGAATAGATGGCA | |
| | | | | | TGTGGCTGGGCAGGTGGATTACAAGGTTAGGAGTT | |
| | | | | | TGAGACCAGCCTGGCCAACATGGTGAAACCCCGTC | |
| | | | | | TCTACTACAAACACAAAAAATTTAGCCGGGCGTGG | |
| | | | | | TGGTGCATGCTGTAATCCCAGCTATTCAGGTGGCTG | |
| | | | | | AGGCAGAATTGCTTGAACCTGGGAGGTAGAGGTTG | |
| | | | | | CAGTGAGCCGAGATGACACCACTGCACTCTAGCCT | |
| | | | | | GGGCGACAGAGTGAGACTCTGTCTCAAAATT | |
| 31 | | A1157402 | 31 | | ATCTTACACACTGTGTGCCCTTTAACACAGATTTA | |
| | | | | | TCTTGACTGATTTATGCTTTTGCTGTCTTTTAATCAT | |
| | | | | | AGACAAAGTAAAAGCATTTCTAAACC | |
| 32 | | Ac022795 | 32 | | AGACTTAACCCTAACATACTACCAATAATGACATTA | |
| | | | | | AATGGAAATTAAATGGAATACCAATCAAAAGAGGT | |
| | | | | | GGTAGGGGTAGATTTTTTTAAATCCCCCATTTATAT | |
| | | | | | ATCTGTCAGAAACTCTTCAAATATAACAATATAGGC | |
| | | | | | AAGTTGAACATCGGAAGATGTGAAGAGATAACATA | |
| | | | | | ACAAATATTAAAAAGAAAGCAGCATATTGGCAATG | |
| | | | | | TTAATACCAATTAAAGTAGACTTCAGAG | |
| 33 | | Ac015564 | 33 | | AGTAGAATCAAAAATTTTTAGAGTCAGTATACTCAT | |
| | | | | | GTAAGCTAACATAAATGAGAAAGAGAGAGAGCGA | |
| | | | | | GAGAAAGAAAGGAAAGGAGGAAGTGGGAAGGGGA | |
| | | | | | AAAGAGGGGAGAGGAGTGGAGGGAGGGGAGGGGA | |
| | | | | | GGGGAGGGAGATACTCTTACTCAGAAATTTTCTTTC | |
| | | | | | TTTGAAAATCCCTTATGACATTTCTAAGAAGAAGCA | |
| | | | | | AGAATAGTGTGACCTTTGCAAATTACCTTAAAGACA | |
| | | | | | AAGAGGAGAAGAAAGAGCCAAGCTAATACATGAA | |
| | | | | | GAGGGAAAACAACCAGAAAAAATGACATTTCAGAC | |
| | | | | | ACAATCATGGACAGAAATCCTACAAGTCAGTAGGG | |
| | | | | | GCCACCTTTACCTGCCAGGGGGACCACAAAAATAG | |
| | | | | | GGGATTTCTGTCAAGAAGGCAGGAATGTTCAGCAG | |
| | | | | | AACACAGCTTCTGAATCATCTGACTCTCTCAGAACC | |
| | | | | | AAGACAAAACAGTTCAAATGCCTACAAGCCACAGG | |
| | | | | | ACCCAGGAAATACCGCAGAGTGGACACTTTCCCCC | |
| | | | | | TCTACATAAAAAGAACCTATTTCTTTTCTATGCATCA | |
| | | | | | GCTTCTCCAGTCCATCTTTTCATTAAAAGGACTGCC | |
| | | | | | ATGGAATGAAAAACTCATATTTCAGGACTAAGATGG | |
| | | | | | ACAACAGGCCTTCTCCAGCTCTTCTCTGAAAAGTGA | |
| | | | | | GCTTTTCGGTAGAGAACGAGCTTCCTTCACAAGAAG | |
| | | | | | GGCACTCCCGCTGGGTGTGAGCCAAACGCACATGC | |
| | | | | | ACGACACTTGCGCAGCTAAGAATACGCACAGTGGG | |
| | | | | | GAAAAGGCACAGAAGCAGCCCCCGTCCTGCCCGAG | |
| | | | | | TGCCACATCCCTTTCTGGGCTTTCATTCCCCCACCCC | |
| | | | | | CACCGCCTGCAAAATGAAAGAAAGATTTGCAATAAA | |
| | | | | | CAAGGTGTAAGTCTCAAACCTGCTCTTCACCTGGAG | |
| | | | | | CTTGTAATCAGGTGTCAGGCTCCCATCCACCCACAA | |
| | | | | | GGAACAGAGAGATTTTGGTGTTGAAGCTTCAACCT | |
| | | | | | GCCCTGCGAGCCAATCTTTATTTCAAAGTACTTTGT | |
| | | | | | GCTGTAAGCTAACGGGAAAAAATGATCAAATGCCT | |
| | | | | | CAAATCTCCCGTAAGCAGGGACTGTGCCTGGGGGG | |
| | | | | | AAAGGTGCTCACCAAGGTGGGGGCACATCGGGTGT | |
| | | | | | CTCCTGGTGCTTTCTGCTGGCACTAACATTCTAAAA | |
| | | | | | CATGAAGCAAAGTACAGCAACATGGATCTTCCTT | |
| | | | | | TTTTAACATGGAAAATACGTTTTCATAGAGCAGGAG | |
| | | | | | GGAAAAGAACTCTCTAAAAAACAGAGCTGAATAGG | |
| | | | | | CTTAGCAAGAAAAGAAATTCAGGAGATGGAGAGGA | |
| | | | | | GGAGCTCTAAAACATCCACAAAAAAATAAACCATT | |
| | | | | | TCATAGCAATGCTGACCATTTTAATTGATTCTCGAC | |
| | | | | | GACAGAAGAACACAAGAAAAGGTAGATGATGTAAT | |
| | | | | | GCGATGGCTGCTGAAGGCAAAAGTCACAAAACAAA | |
| | | | | | TTTAGCCCTTCGAATACCACAGTAGCCATGGGTCAA | |
| | | | | | TATAAAAAGCTTTTCAACGGTCAGGAGCAAAACTGGG | |
| | | | | | GTGAAGGGGCTACTCCCCCATACATGTAATTTGTCC | |
| | | | | | AAGCCCTGCCATAGCCACCACCTCCCTGGATCCTCA | |
| | | | | | AAGCAACCCTATTATGCAAGACATGCTGATCCAGG | |
| | | | | | TGCATCTGACGATTCAGAAAACCAGGACCAAGCCG | |
| | | | | | TGGGGCACCGAGCCTGAGCTAATAAGCAGCAGAGT | |
| | | | | | CGACCCTGGCACGAAGGTCTCCCAGCTCCATGAAG | |
| | | | | | ATGCATCATCAAGAAGGTTGGGCCTCAAATTCTTTC | |
| | | | | | CATTACACTTCATGTTTCTCCCTGGATTATCTCCATA | |
| | | | | | AAGGAGAAAAACAATACCCAGAACACAATTCCAAC | |
| | | | | | TCTGAGAAATTGTCTGATCTTCCTCCTTGTCTCTGCC | |
| | | | | | CCT | |
| 33 | | Ac015564 | 269 | | TTTTTTGAGACAGGGTCTTGCTCTATTACCCATGCT | |
| | | | | | GGAGTGCAGTAGTAGAATCAAAAATTTTTAGAGTC | |
| | | | | | AGTATACTCATGTAAGCTAACATAAATGAGAAAGA | |
| | | | | | GAGAGAGCGAGAGAAAGAAAGGAAAGGAGGAAGT | |
| | | | | | GGGAAGGGGAAAAGAGGGGAGAGGAGTGGAGGGA | |
| | | | | | GGGGAGGGGAGGGGAGGGAGATACTCTTACTCAGA | |
| | | | | | AATTTTCTTTCTTTGAAAATCCTTATGACATTTCTA | |
| | | | | | AGAAGAAGCAAGAATAGTGTGACCTTTGCAAATTA | |
| | | | | | CCTTTAAAGACAAAGAGGAGAAGAAAGAGCCAAGC | |
| | | | | | TAATACATGAAGAGGGAAAACAACCAGAAAAAAT | |
| | | | | | GACATTTCAGACACAATCATGGACAGAAATCCTAC | |
| | | | | | AAGTCAGTAGGGGCCACCTTTACCTGCCAGGGGGA | |
| | | | | | CCACAAAAATAGGGGATTTCTGTCAAGAAGGCAGG | |
| | | | | | AATGTTCAGCAGAACACAGCTTCTGAATCATCTGAC | |
| | | | | | TCTCTCAGAACCAAGACAAAACAGTTCAAATGCCT | |
| | | | | | ACAAGCCACAGGACCCAGGAAATACCGCAGAGTGG | |
| | | | | | ACACTTTCCCCCTCTACATAAAAGAACCTATTTCTT | |
| | | | | | TTCTATGCATCAGCTTCTCCAGTCCATCTTTCATTAA | |
| | | | | | AAGGACTTGCCATGGAATGAAAACTCATATTTCAG | |
| | | | | | GACTAAGATGGACAACAGGCCTTCTCCAGCTCTTCT | |
| | | | | | CTGAAAAGTGAGCTTTTCGGTAGAGAACGAGCTTC | |
| | | | | | CTTCACAAGAAGGGCACTCCCGCTGGGTGTGAGCC | |
| | | | | | AAACGCACATGCACGACACTTGCGCAGCTAAGAAT | |
| | | | | | ACGCACAGTGGGGAAAAGGCACAGAAGCAGCCCCC | |
| | | | | | GTCCTGCCCGAGTGCCACATCCCTTTCTGGGCTTTC | |
| | | | | | ATTCCCCCACCCCCACCGCCTGCAAAATGAAAGAA | |
| | | | | | AGATTGCAATAAACAAGGTGTAAGTCTCAAACCTG | |
| | | | | | CTCTTCACCTGGAGCTTGTAATCAGGTGTCAGGCTC | |
| | | | | | CCATCCACCCACAAGGAACAGAGAGATTTTGGTGT | |
| | | | | | TGAAGCTTCAACCTGCCCTGCGAGCCAATCTTTATT | |
| | | | | | TCAAAGTACTTTGTGCTGTAAGCTAACGGGAAAAA | |
| | | | | | ATGATCAAATGCCTCAAATCTCCCGTAAGCAGGGA | |
| | | | | | CTGTGCCTGGGGGGAAAGGTGCTCACCAAGGTGGG | |
| | | | | | GGCACATCGGGTGTCTCCTGGTGCTTTCTGCTGGCA | |
| | | | | | CTAACATTCTAAAACATGAAGCATTAAGTACAGCA | |
| | | | | | ACATGGATCTTCCTTTTTTTAACATGGAAAATACGTT | |
| | | | | | TTCATAGAGCAGGAGGGAAAAGAACTCTCTAAAAA | |
| | | | | | ACAGAGCTGAATAGGCTTAGCAAGAAAAGAAATTC | |
| | | | | | AGGAGATGGAGAGGAGGAGCTCTAAAACATCCACA | |
| | | | | | AAAAAATAAACCATTTCATAGCAATGCTGACCATTT | |
| | | | | | TAATTGATTCTCGACGACAGAAGAACACAAGAAAA | |
| | | | | | GGTAGATGATGTAATGCGATGGCTGCTGAAGGCAA | |
| | | | | | AAGTCACAAAACAAATTTAGCCCTTCGAATACCAC | |
| | | | | | AGTAGCCACGGGTCAATATAAAAAGCTCAACGGT | |
| | | | | | CAGGAGCAAAACTGGGGTGAAGGGGCTACTCCCCC | |
| | | | | | ATACATGTAATTTGTCCAAGCCCTGCCATAGCCACC | |
| | | | | | ACCTCCCTGGATCCTCAAAGCAACCCTATTATGCAA | |
| | | | | | GACATGCTGATCCAGGTGCATCTGACGATTCAGAA | |
| | | | | | AACCAGGACCAAGCCGTGGGGCACCGAGCCTGAGC | |
| | | | | | TAATAAGCAGCAGAGTCGACCCTGGCACGAAGGTC | |
| | | | | | TCCCAGCTCCATGAAGATGCATCATCAAGAAGGTT | |
| | | | | | GGGCCTCAAATTCTCCATTACACTTCATGTTTCTC | |
| | | | | | CCTGGATTATCTCCATAAAGGAGAAAAACAATACC | |
| | | | | | CAGAACACAATTCCAACTCTGAGAAATTGTCTGATC | |
| | | | | | TTCCTCCTTGTCTCTGCCCCTCAAAAAAAA | |
| 34 | | Ac022862 | 34 | | CTATCCAGTAGTATATCTGAGTAAATCCTGTCCCT | |
| | | | | | CAGTAGATCATCTCTTGGGATCTGGTTTCTTGATCT | |
| | | | | | GTATTTCAATATATTCTATATTCCATATAGATCAAG | |
| | | | | | ACTTTCTAACATAAAGCAGTGTGGAATAGACTTACT | |
| | | | | | TTTTATCTTCTCTGTTACTCTTTTGATTTGTGACTTTT | |
| | | | | | ACCAATTTATTGAACTTCTTAAGTGTCAGTGTTTTTA | |
| | | | | | ATCCATTTAGGTTATCGCCAAGGCCTCTAAAAGCTCT | |
| | | | | | AAGATTCAGTGATATGAATACATATTTGCAGTATTA | |
| | | | | | GAGACATTGTACTGTTTTCACTTGGCTTCTAGGACA | |
| | | | | | TTAGATTTTCTATTCTCCCTTTCCTATGCTCACTCCC | |
| | | | | | AGATTCCTTAACCAGTTCCTTGCATCTTTGTGTATTA | |
| | | | | | GAATGCCTCAGGGATAAGTCTTGGATTTCTGCTCCT | |
| | | | | | TTCTAGCTGCACTCACTTCCTTGGTAAGCTCATCTG | |
| | | | | | ATTTCATCATAACTTCACCTTTACATACTGCAAACT | |
| | | | | | CACAAATTATCTTTCCCTGAACTTGAGACTCCTATCC | |
| | | | | | TGCTGCCTGCTTATCATCTTTACTTGACTATATAACG | |
| | | | | | AACATATCAAACATAAACTGAACTGATAGTCTCCTA | |
| | | | | | ACCTGAAACCTGCTTCTATAGTCTTCCCCAACTAAG | |
| | | | | | TTATTGGCAAATACGTCCTTGCATTTTCTCAGGCCA | |
| | | | | | AAATCACATCATGATCCTTGGCATTTCTTTCTCTGGT | |
| | | | | | ACCCCATGCCCTGTCTGCAGATCTATTGGCAAAACC | |
| | | | | | TCCCAACATCTTAACAGCAGCTTTACTACCACACTT | |
| | | | | | TTCCAAACGGATTACCTCTAGCCTGCATGATTGCAT | |
| | | | | | TAGTCTGCCTCCCTGCTTCTGGCTTTTACCTACTCAG | |
| | | | | | GCTATCCCAGCACCCAGAATGACAACTTTGAAAA | |
| | | | | | CAAAGCTTGCCGCCACGTGCAGTGGCTCATGCCTGT | |
| | | | | | AATTCCAACGCTTTAGAAGGCGGAAGTGGGCAGAT | |
| | | | | | CGCTTGAGGTCAGAAGTTTGAGACCAGCCTGGCCA | |
| | | | | | ACATGGTGAAACCCCATCTCTACCAAAAATAAATA | |
| | | | | | AATTAGCTGGGCATGGTGGTGCATACCTGTGATCCC | |
| | | | | | AGCTACTTGGGAGGCTGAGGCAGGAGAATCGCTTG | |
| | | | | | AACCTGGGAGGCGGAAGTTGCAGTTAGCAGAGATC | |
| | | | | | ATGCCATTGCACTCTAGCCTGGGCGACGGAGTGAG | |
| | | | | | ACCCCATCTC | |
| 35 | | A1035683 | 35 | | GTATGTTAATGTATGTAATGCATAGTATGAGTATCC | |
| | | | | | AGCATTTTAAGCAGATTTAAAATGGAAAAATTCAT | |
| | | | | | GATTCACATTAGAGCTTCAAACTTATAAAATTTGGG | |
| | | | | | GGATGCATTATAGCGTGAGTATTGGCACCCACTCCT | |
| | | | | | GAAGTGGAATATTGGAAGCCTGAAATATATGACAT | |
| | | | | | GTTGACAGTAAAGATCCAGGTAATATTGGCCATGC | |
| | | | | | GGGGTGGCTCACACCTATAATCCCAGCACTTTGGGA | |
| | | | | | GGCCAAAGTGTGAGGACTGCTTGAGCCAGGGAGGT | |
| | | | | | TAAGACTGCAGTGAGCCATGATCGTGCCACTGCACT | |
| | | | | | CCAGCCTGAGTGACAG | |
| 36 | 116917 | | 36 | | CTTGTTGGCACTGAGGTACCGGTTTGGAATTCCCGA | |
| | | | | | GCGTCGACGGGGGGAAAAATAAGAGGAATGAATAT | |
| | | | | | TTTAAGCTTGCTATATAATTAAAATATTCTTAGAA | |
| | | | | | GTCTGGAGTCTGTGAAGGTCACACCCTCTGGTCTC | |
| | | | | | TCCCAGCCCATAGGGTATAAATAATCTGATTGACG | |
| | | | | | GCATCCAGGGATCTCAGAAATTATTAGTACATCCCA | |
| | | | | | CAGTGAATACCACCTTACTAAAATATTCATGGGTA | |
| | | | | | TATACTATGGATTTGTTTTATCCTATTTAGTCTTAAA | |
| | | | | | AACTATAAAGAAATCTGCAGGCTTATTTAACATATA | |
| | | | | | CTCAGAATCATATTGTCTCCAAAGCACAAACTGAAT | |
| | | | | | CAGTTACAAGATATTGGACTAGAGATCATGGCAAA | |
| | | | | | TCAGAGGTACATAAGACCTAGTTCCGTTGTGGAGCT | |
| | | | | | AAACAAACTGCAGAGACCTAAAGGGAAGCCTTGCA | |
| | | | | | CCACACTCTAGGTTTGGAGCTCAGGTTTTGAGTGGT | |
| | | | | | GTCAGCACTCCAGAACACATGGGATCCCCGGGAGG | |
| | | | | | TGGAAATTGAGCCGTCTTTGGAGAATCAGCTAATG | |
| | | | | | AGACAGATGCATGTTAAATGTCTGTTGTGGCCCAGG | |
| | | | | | CACTCTGCTAGGCAGAGGGGTGAACCAGAAGAATG | |
| | | | | | AGATTCATGGGGCCAAAGAATTTGCCTTCTGGTGTA | |
| | | | | | AGAAAAGATGGAGGCAGCTTG | |
| 36 | 116917 | | 20 | | CTTGTGGCACTGAGGTACCGGTTTGGAATTCCCGA | |
| | | | | | GCGTCGACGGGGGGAAAAATAAGAGGAATGAATAT | |
| | | | | | TTTAAGCTTTGCTATATAATTAAAATATTCTTAGAA | |
| | | | | | GTCTGGAGTCTGTGAAGGTCACACCCTCTGGTCTTC | |
| | | | | | TCCCAGCCCATAGGGTATAAATAATCTGAATTGACG | |
| | | | | | GCATCCAGGGATCTCAGAAATTATTAGTACATCCCA | |
| | | | | | CAGTGAATTACCACCTTACTAAAATATTCATGGGTA | |
| | | | | | TATACTATGGATTTGTTTTATCCTATTTAGTCTTAAA | |
| | | | | | AACTATAAAGAAATCTGCAGGCTTATTAACATATTA | |
| | | | | | CTCAGAATCATATTGTCTCCAAAGCACAAACTGAAT | |
| | | | | | CAGTTACAAGATATTGGACTAGAGATCATGGCAAA | |
| | | | | | TCAGAGGTACATAAGACCTAGTTCCGTTGTGGAGCT | |
| | | | | | AAACAAACTGCAGAGACCTAAAGGGAAGCCTTGCA | |
| | | | | | CCACACTCTAGGTTTGGAGCTCAGGTTTTGAGTGGT | |
| | | | | | GTCAGCACTCCAGAACACATGGGATCCCCGGGAGG | |
| | | | | | TGGAAATTGAGCCGTCTTTGGAGAATCAGCTAATG | |
| | | | | | AGACAGATGCATGTAAATGTCTGTGTGGCCCAGG | |
| | | | | | CACTCTGCTAGGCAGAGGGGTGAACCAGAAGAATG | |
| | | | | | AGATTCATGGGGCCAAAGAATTTGCCTTCTGGTGTA | |
| | | | | | AGAAAAGATGGAGGCAGCTTGGCAGAAAAAAAAA | |
| | | | | | AAAGGTAAAAGATAGAAATGAAATACAGATGTGAG | |
| | | | | | GCACCGTATCCAGGCTGTATGGAGTCTTTCTAATCA | |
| | | | | | GGACATAGGCAGACAGTCCTAGCCCAGCTTTATGC | |
| | | | | | CTTATGAGACGCAACAACGTTTGAACAGTCCTTGTT | |
| | | | | | TGAGGGACCAGAGGTTTTACCAGATGGATGATAAC | |
| | | | | | TAGCATCTGTGGAACATATTTGTGAAATATAGAAA | |
| | | | | | TCAGAAATTCCCAGCGTAGCACTGTCCCAAGGGGA | |
| | | | | | ACATAATTTGACCTGCATATTTGCTGGTCCATTTTTA | |
| | | | | | GTAGTCACATTAAAAAAGAAAAATGACACAGGTGA | |
| | | | | | AATTAATTTGAATATATTTTCTTAATTCAGTATGCTT | |
| | | | | | AAATATTATTAAGTATGTACTCAATATAAGCAATT | |
| | | | | | GTTTAATGAAATATTTTACTCTTTTTGAACTATGTGTT | |
| | | | | | TGAAACCCCGGATGTATTTTTTTTTTATCTTCACCAC | |
| | | | | | ACATTTCAATTTGGGTTGGTCACATTTCAAGTGCTC | |
| | | | | | AGGAGTCACATGCAGCTAGGGGTACCTATTGGAC | |
| | | | | | AGGCAGGCAGATCTTGAGAGCTCCAAAGAACTGTG | |
| | | | | | TGTCATTATATTGTGG | |
| 37 | 22946 | | 37 | | CAACAAGGTAGGCCCAGGGAAGGGGTTTGTAGGGA | |
| | | | | | GGTGGAATAGGATAGGGGAAGGGAGGAGGCACTG | |
| | | | | | AGCGACAGTGAAATCAGGACAGGACGTGGAGAGG | |
| | | | | | ATGAGGTGTGTGGGAGAGAGCAGAAGGGCTTTAAT | |
| | | | | | TCTGAGACCTGGGATTATAAAGCCCCAAGAGGGGA | |
| | | | | | GGCTGGGAAGTGCCGGCCCTCAAATGTCCTTACTCT | |
| | | | | | GCACAGACCTAGCAAGGGCTCTGCCTGCCCCTGGC | |
| | | | | | CGGGTGTGGACATGGAGAAGGGGAGCCAAGAGGT | |
| | | | | | ACGTTCTTGTGAGGCGCCTTCTCCTCGGAGCCCGTC | |
| | | | | | CCGCAGATGTGGACTCACAGCCGCCCACCTGGTCC | |
| | | | | | ATGTGCCTCCGCAGCCTGGACCGGTTCCCTCCTCTG | |
| | | | | | CGGGGCGGAGACCAGAACACAGACTTCCTGAGACT | |
| | | | | | GAGTAATAATAGGAAGGATGTGATTTCCATAATGG | |
| | | | | | AAATAATGGAACAAGGAAATGATCCTCCTTATTATT | |
| | | | | | ATCTCCAAGGGACAGCGTGGGAAAATACAGCAGCT | |
| | | | | | TCTCCTACCTAATAAGAAGAAAATGAGTATATAAA | |
| | | | | | AATGTACTGCAGTTTGGCCCAGGGGCTCACGCCTGT | |
| | | | | | AATCCCAACACCTTGGGAGACCAAAGTCGGGGGAT | |
| | | | | | AGCTTGAGCCCAGGAGTTCGAGACCATCCTGGGCA | |
| | | | | | ACATGTCAAGACCCCATCTCTACAAAAGAAAAAAA | |
| | | | | | TTTTTTTTAATTAGCCAGGTGTGGTGGCACACCTGT | |
| | | | | | AGTCTGAACTACTCGGAAGGCTGAGCTGGGAGGAT | |
| | | | | | CGCTTGAACACGGGAGGGAGAGGCTGCAGTGAGCC | |
| | | | | | AAGATCACACCACTGTGCTCCAGCCTGGGCGACAG | |
| | | | | | AGCAAGACACTG | |
| 38 | 206416 | | 38 | | GTGTTGCATCTGCAGTGCCACTAGAACAAGGATAG | |
| | | | | | CAGACTGAGGTGGTAGAAAGCAGACTCAACAGGGC | |
| | | | | | AAAAGGCAAGAGATCTGTTTCAAGTGCAAGGGCCT | |
| | | | | | TGAGCCTTTTGTCCAGTGGCAGGATGGGGTGGGGT | |
| | | | | | GAGCAGGAGACAGGTGGCTAGTGTGATAAAGAGTA | |
| | | | | | CGGGGCCGGTTGGAGAAGAGTCATTAGAAAAAGCC | |
| | | | | | TCTCTGAGGAAGTGACCTTTGAGCTGAACCAGCAC | |
| | | | | | GGGGAGAGCACAGAGAAGAACTCAGCAAATACAC | |
| | | | | | AGAAAGCACATATCACATGCAAAGGCCCTGGGGCT | |
| | | | | | AGAGTGAATTTGATGATCAAGAGACAGTGAGTAGA | |
| | | | | | GGATGGGTCAGTAGGTGTGCAGCAAACCACCATGG | |
| | | | | | CACATGTATACCTGTGTAACAAAACCTACACGTTCT | |
| | | | | | GCACATGTATCCCAGAACTTAAAGTGGAAGAAAAA | |
| | | | | | AAAGGGGAAAGAAGGAAGGAAGGAAGGAGAAAGA | |
| | | | | | AAGAAGGAAGGAAGGAAACAAAGGTAGGTATAAT | |
| | | | | | GACACGGCCGGGGGAACCCTC | |
| 39 | 1137189 | | 39 | | TGGCTGAAAACTTTAAAAGCTCAGGTTAGTTCAGAT | |
| | | | | | AGATTCAGGGTGAGCTGAAAGCCAGCCCCCTGGCC | |
| | | | | | CTGCGGTGACTTTTTCCAAAAGATAAATGAGTGAG | |
| | | | | | GCCAGGAGTGTCATGCAGACGGGCTTTGGGCCGGC | |
| | | | | | TATGGGTGTTGGCATCTTGTTTTGAAACCCCCTTCC | |
| | | | | | ACATCTGCTCAGGGGTCACAATCTTAAGTGCTGAAG | |
| | | | | | GGGTGCAGCTGACGAATGAGAAAAGCAGACAGTGT | |
| | | | | | GGAGCCTGGGGAGCTGGTCCTTGCCTCGTCCTTCAC | |
| | | | | | CATTTGTTGCCCTGTGGGAGTGCTAAGTTAGTGTTT | |
| | | | | | CCAGATCTTCTGATTGTTAAGAGAGGCTGGAAATCC | |
| | | | | | GTATTTTTCAAGAGGATTGAGTTGCCAACTCATTGA | |
| | | | | | AATCTTCTCCAAGCCCCTTGCGAGTCAGCATTGGTT | |
| | | | | | AGCATGTCTCGAACACATGGTAGCTCAAACACACA | |
| | | | | | CGGTAGCTTGCCATGGTGGCAATTTCAAATTGCATT | |
| | | | | | CATTGATTTCAAAAGACCATCAATTTCAAATTGCAT | |
| | | | | | TCATCTTTTTGAGTTGCGAAATAAT | |
| 39 | 1137189 | | 271 | | TGGCTGAAAACTTTAAAAGCTCAGGTTAGTTCAGAT | |
| | | | | | AGATTCAGTGTGAGCTGAAAGCCAGCCCCCTGGCC | |
| | | | | | CTGCGGTGACTTTTTCCAAAAGATAAATGAGTGAG | |
| | | | | | GCCAGGAGTGTCATGCAGACGGGCTTTGGGCCGGC | |
| | | | | | TATGGGTGTTGGCATTCTTGTTTTGAAACCCCCTTCC | |
| | | | | | ACATCTGCTCAGGGGTCACAATCTTAAGTGCTGAAG | |
| | | | | | GGGTGCAGCTGATGAATGAGAAAAGCAGACAGTGT | |
| | | | | | GGAGCCTGGGGAGCTGGTCCTTGCCTCGTCCTTCAC | |
| | | | | | CATTTATTGCCCTGTGGGAGTGCAAAGTTAGTGTTT | |
| | | | | | CCAGATCTTCTGATTGTTAAGAGAGGCTGGAAATCC | |
| | | | | | GTATTTTTCAAGAGGATTGAGTTGCCAACTCATTGA | |
| | | | | | AATCTTCTCCAAGCCCCTTGCGAGTCAGCATTGGTT | |
| | | | | | AGCATGTCTCGAACACATGGTAGCTCAAACACACA | |
| | | | | | CGGTAGCTTGCCATGGTGGCAATTTCAAATTGCAT | |
| | | | | | CATTGATTTCAAAAGACCATCAATTTCAAATTGCAT | |
| | | | | | TCATCTTTTGAGTTGCGAAATAATAAACACGAAAA | |
| | | | | | AAAAAAAAAA | |
| 40 | 7248 | | 40 | | CAGGAAGACCCTCTCAGAAAAAAAAAAAAAAGAA | |
| | | | | | TTTGGCCGTTATGTGGAGGACTGGAATTGAGAAGG | |
| | | | | | GCAAGAGCGAGGTAGAAGAGTGGTCTAGGGAGAA | |
| | | | | | CAGTTAGGGGCTATTGCAATTATCCAGCAAGAGAT | |
| | | | | | CTTGGACCAGGATGGCAGCAGTGGAGGTGGTAAAA | |
| | | | | | TGTGGTGGATGAAGCGTACGCTTTGAAGGTATCAA | |
| | | | | | CAGGACCAGCTGATGGAAGGGAGTCAACAGGACTA | |
| | | | | | GCTGATGGCTGTAAACTGGGGGGTCACTAGCTATC | |
| | | | | | AGATGGCATTTACTTAAAGCCATGGAAGTAGGTGA | |
| | | | | | GCTCCCTTATGGAGAGGGAATAGGAAGGAGGTAGA | |
| | | | | | CCATTCTATCAAAATGCTCTTTCTACAGGGCACTTC | |
| | | | | | TCACTGAGATATTATTTATCTGGGATTTATATTATTT | |
| | | | | | ATTCAATTTGTTTTGTGTTTGGTTCTATTAGAAAAGC | |
| | | | | | TCCATAGG | |
| 40 | 7248 | | 279 | | AATAAGTACATCAGACAACAAGTCAAGTCAAGTCT | |
| | | | | | TGCCTCATGGAGCTAACATTTCTAAGAGGAGAAAC | |
| | | | | | ATGCAGTAAACAAGTAAAGAAATGTATGCTCTATT | |
| | | | | | CAGGGAGTAGTTTGTGCTATGAGGAAAAGCAAAAC | |
| | | | | | AGGTTGAAGAGATAGCTATGTGGTGGGAGTGGGAC | |
| | | | | | TATTTCGTACAGGGCACTGATTGTAGACCTCTGATG | |
| | | | | | AGATAACATTTGACAAGAGATCTGCAGGGAGCTAT | |
| | | | | | GTGTCATGGGGGAAGGCATTGGAGGGTTTTGTGCA | |
| | | | | | GGACAGTGATGTGTGATCAGATTTAGTTTAAAAGA | |
| | | | | | ATAATTTGGGCTGGGCATGGTGGTTCCTGCCTGTAA | |
| | | | | | TCCCAGCACTTTGGGAGGGTGAGGTGGGCGAATCA | |
| | | | | | CTTGAATCTGGGAGTTTGATACCAGTTCGGGCAACA | |
| | | | | | TGGCGAAATCCCGTCTCTACAAAAAATACAAAAAT | |
| | | | | | TAGCCAGTGTGGTGGCACGCGCCTGCAGTCCCAGCT | |
| | | | | | ACTTGGGAGGCTGAGGTGGGAGAATTGCTTGGATC | |
| | | | | | TGGGAGGTGGAGGTTGCAGTGAACTCAGATTGCGC | |
| | | | | | CACTGCACTCCAGCCTGAGATTGTGCCACTGCACTC | |
| | | | | | CAGCCACTGCACTCCAGGAAGACCCTCTCAGAAAA | |
| | | | | | AAAAAAAAAAGAATTTGGCCGTTATGTGGAGGACT | |
| | | | | | GGAATTGAGAAGGGCAAGAGCGAGGTAGAAGAGT | |
| | | | | | GGTCTAGGGAGAACAGTTAGGGGCTATGCAATTA | |
| | | | | | TCCAGCAAGAGATCTTGGACCAGGATGGCAGCAGT | |
| | | | | | GGAGGTGGTAAAATGTGGTTGGATGAAGCGTACGC | |
| | | | | | TTTGAAGGTATCAACAGGACCAGCTGATGGAAGGG | |
| | | | | | AGTCAACAGGACTAGCTGATGGCTGTAAACTGGGG | |
| | | | | | GGTCACTAGCTATCAGATGGCATTTACTAAAGCCA | |
| | | | | | TGGAAGTAGGTGAGCTCCCTTATGGAGAGGGAATA | |
| | | | | | GGAAGGAGGTAGACCATTCTATCAAAATGCTCTTTC | |
| | | | | | TACAGGGCACTTCTCACTGAGATATTATTTATCTGG | |
| | | | | | GATTTATATTATTTATTCAATTTGTTTTGTGTTTGGT | |
| | | | | | TCTATTAGAAAAGCTCCATAGGGGCCGGGCACGTT | |
| | | | | | GGCTTTTGCCTGTAATCCCAACACTTGGAAGGCCG | |
| | | | | | AGGCAGGCGGATTACCTGGGGTCAGGAGTTTGAGA | |
| | | | | | CCAGCCTGGCCAACATGGTGAAACTCTGTCTCTACT | |
| | | | | | AAAAACACAAAAATTAGCCGGGCGTGGTGGTGCGC | |
| | | | | | CTGTAATCCCAGATGCTGAGGAGGAGAATCGCTTG | |
| | | | | | AACCCGGGAGGTGGAGGTTGCAGTGAGCCGAGATC | |
| | | | | | GCGCCACTGCACTCCAGCCTGGGCAACAAGAGCGA | |
| | | | | | AACTCCCTCTCAAAAACAAACAAACAAACAAACAA | |
| | | | | | ACAAACAAAAAACAAAAAAAAGAAAGAAAGAAAG | |
| | | | | | AAAAGGGCCAGGTGTGGTGGCTCACACCTGTAATC | |
| | | | | | CCAGCACTTTGGGAGGCTGAGGCAGGCGGATCACG | |
| | | | | | AGGTCAGGAGATCGAGACCATCCTCACCAACACGG | |
| | | | | | TGAAACCCCGTCTCTACTAAAAATACAAAAATTAGT | |
| | | | | | CGGGTGTGGTGGCGGGCGCCTGTAGTCCCAGGTAC | |
| | | | | | TCCGCAGGCTGAGGCAGGAGAATCGCTTGAACCCG | |
| | | | | | GGAGGCGGAGGTTGTAGTGAGCCGAGATTGAGCCA | |
| | | | | | CTGCACTCCAGCCTGGGTGACAAAGTGAGACTCCA | |
| | | | | | TCTCAAAAGAAAAAAGCTCCATAGGAGAAGGAACC | |
| | | | | | TTGTCTCTCACCACATAAACTGTGTTTGGATTCGC | |
| | | | | | AATCGAGTTGGGAAAAAAAAATCAGTCTGGAAGAG | |
| | | | | | CCACACCAAACCGCTAACAGCTACTGTCTCTGGGA | |
| | | | | | ATAGAACAAGGAGTTTGGTTGGCGCGATATACCGC | |
| | | | | | CCCTGAACCTCTAGCCACAATAAGGCTTAATTAATG | |
| | | | | | ACCGGACGACTTGAAAGCGCCTCCACTGTTTATCT | |
| | | | | | CTTAAATCTGCAACGAAATGCAACAAAAACGCAAG | |
| | | | | | AAATAAACAATAGAAGCCAGTCTTACTGCACACTG | |
| | | | | | CAGAAGCCAATAAACCCCAAATGTAGCTCAAAACA | |
| | | | | | AGGTGTCACGCAAACTTCTGATTTTTTTTTGTTTTAC | |
| | | | | | ACTGAATCTCTGTCACTCTGACTAGAGGGCAGTGGC | |
| | | | | | GCGATCTCGGATCACTACAACCTCCGTCTTCTAGAG | |
| | | | | | TCAAGAGACTCTCCCGCCCCAGGAGTCTCTGCCACT | |
| | | | | | CTGACTAGAGGGCAGTGGCGCGATCTCGGATCACT | |
| | | | | | ACAACCTCCGTCTTCTAGAGTCAAGAGACTCTCCCG | |
| | | | | | CCCCAGCTTCTCCAATAGGTGGGATTGCAAACAGG | |
| | | | | | CACCACCACGCCCGAATAAGTTTGTGAACATTTGGT | |
| | | | | | ACAAATAACAAATGACCAAGTTCCTTGGTTGTAGTT | |
| | | | | | GCCTAACTTTTAATACTTAAAAATGTAGCCTCAGGA | |
| | | | | | AATAAGAGGCCTCAAAAAATTGAATAAAAACTCAC | |
| | | | | | AACTTTCTCTCCACGGAAATCTTTAGTAAAAGGCGA | |
| | | | | | AAGATTTATGCGCTTTGAAGAGAAACCCGAGTATA | |
| | | | | | TTCGTGACTTCCGCTTCGAACCTCGCAGGGAGAACT | |
| | | | | | AACACTTAACAQCACTTATGGTTGTTGGATGCCTGCG | |
| | | | | | TGGTACGCACTCCCTATATGTAGTTTATGCACACAG | |
| | | | | | ATGCGTGTAAGAGGCATCATGCTCTAAAACAGTGC | |
| | | | | | AGAAATGCGCGCACAGAGGGAGTGCAAGCATCTTG | |
| | | | | | AGGGTATCTTTTCGTGGTGCACCATGGGTATGCAAA | |
| | | | | | TCACAGGCGGCTCCGGGCTGTTCCGCGCCACCGGG | |
| | | | | | GAAGCCATGGGTCACTGATCTCCTTTGCTCTCCTAT | |
| | | | | | GCTCCTCTCTGCTGGTCCTCCTGGGACCCGCACCCC | |
| | | | | | GTGGGCGGCGCC | |
| 41 | 1101000 | | 41 | | GAAAATTGTTTTTAAGTAACTTTATTGTATACCAAA | |
| | | | | | ACAAAGCTCAAAGAATTTTAACACAAAATGCAAAA | |
| | | | | | AAATCCAGCACCCAATAAGTACAATGCTCAATGTC | |
| | | | | | TAACCCCAAATAAAATAATGTTAGGAATGCAGAGA | |
| | | | | | AACAGAAAACTGTAATCCATGATAAGAAGGGGGAA | |
| | | | | | AAAAATCAATCTACTTAAACTGACTTAGAAAAGAC | |
| | | | | | ACATCAGGTGAGAATTAAAAAACAATAAAAAGGAC | |
| | | | | | ACAGATGAGAGAATCTGTAGATAAGCACATGAAA | |
| | | | | | CAAATATAACTGTATACCTTGTATTAAAGAAGCTAG | |
| | | | | | GCCAGTGTGGTGGCTCATGCTTGTAATCCCAGCACT | |
| | | | | | TTGCCAGGCCAATGTGGGTCACATGAGGCTGATCTC | |
| | | | | | AAACTCCCAACCTCAGGTGATCCTCCCAAAGTGCTG | |
| | | | | | GGATTACAGGCTCAAGCCACCAAGCCTGGCCAAAA | |
| | | | | | AAAATTTCTAATTGCAATTCTGAACAAGTTATGGGT | |
| | | | | | TGTGAAATCAATATAGTGGACTGCTTGCTACTACAG | |
| | | | | | GCTTTATTTAAATACTAGGAAGGTTGGATTACACAT | |
| | | | | | AATGAAAGATTTTTAAAAACTGATCACAAAGAAT | |
| | | | | | TGTATATTTCTCACTGCATCTTGTGGTCAGATAAGT | |
| | | | | | TTGAGAAACAAAACCATGGCGAGAGGAAGGAAAA | |
| | | | | | TTCCATCAATGGGTGGTGTTAAGCCTTTTCTATGAG | |
| | | | | | GTAGCTGTACATTTTGGGACACTTCTATGTGGCGAC | |
| | | | | | TTGACATTCTAATAGATAGATGGTCCTTTCATCTCT | |
| | | | | | AGCCACATGTGAAAATTACTTGGGAGCTTTTTAAGA | |
| | | | | | CTACTAGTGGCTTCCACCCACCTGGAAGCATTTAAA | |
| | | | | | TCAGAATCTCTAGGTGTAGAGTCCAGGCACTTGTGT | |
| | | | | | TAAAACCTCACCAGGCTTTATAATATGACAGAATG | |
| | | | | | GTTTAAAGCTACTGAGTAGACCCACCCTATTTCCCA | |
| | | | | | CCATTCTCTTTGTTTCTCTTTCACCATAGGCTTCTTT | |
| | | | | | CCCATGAGAAAGTAAAGATTTAGTCTCTCTTTTCCA | |
| | | | | | CAGTCTGAAGTAAATCACTATCTTTCTCAACTGGAC | |
| | | | | | TTCCAAGGCAAAGATTTCTTTCCATTTATCTATCTG | |
| | | | | | GAGTTTTACAAAGTTGGCCTCTGGATTCCCTTTTCC | |
| | | | | | CAAAGCTAATTCACCACAAAGGCACCCCTCAAGTC | |
| | | | | | AAGGAGCTGGACTTTCATACACCTGCACCTGTCAAT | |
| | | | | | CATGGGTAAATAATTTGCAGGCAAGGTTGCTGGGT | |
| | | | | | GCTGTGGGATTGACATAAACTCCCAGGTATTGCCAG | |
| | | | | | CTCTGAGCCTCAGGCAAGCTTGTGACTAAATGACTC | |
| | | | | | CAGTAGTCTGAGGACAGTCCTTACTCAGAAGGGTCT | |
| | | | | | TTGGAAGCAAAAGCAGACATAGGCATGAGAGGGT | |
| 42 | 427125 | | 42 | | AATAATAGATAAACCAATGCCATGTGCCTCCTAATG | |
| | | | | | ACATGCACTGAGAAGGATACATCATTGCTGTAATG | |
| | | | | | GTATTTCTGTTTAAAATGTATAACCTGTATCTAAAA | |
| | | | | | TGAGGAAACATCAGATAAATCCAAATTGAGGTTAT | |
| | | | | | TGAGAACAATGATTCTAATTAAATAGTATGAAATA | |
| | | | | | GAGAAAACGTAAGTAAATACTCTATATTCCTGAATT | |
| | | | | | TTAATTGGTGGGAGTATCACTTTGACCAGTCCAGCA | |
| | | | | | GCAATACACATCACTAGCACATACATTATGGTATTT | |
| | | | | | ATGGACCATTTCCTGCTAAAAGAAACCAGGATTTCT | |
| | | | | | TGGGAGAAGTGGCTGATTCCAAGTATGGGCAGAAA | |
| | | | | | ATTTTTAATGAGCCTGCAGTATTTCTCATACCAGA | |
| | | | | | TAATAACAAAGCTAATTTAAAAAATCAGTAGATTA | |
| | | | | | ATGACAAAGCACTGCCAACTTGGAAAGGTTTCCAA | |
| | | | | | TGACCAAGGATAGGACAAATCAAGCTTAAATATAA | |
| | | | | | AAATAATTTATATTTGAAACACACCAAATACATTTA | |
| | | | | | TAGTTGAATAAATACAAATTTACATTTATAGTTGAA | |
| | | | | | TAAATATAAATCTACATTTATAGT | |
| 43 | 14249 | | 43 | | GAAATGACTTCCATAAGGTTGTGCAGCTAGTTTGCA | |
| | | | | | ACAGGTCCCCTGACTTCCAGGCCCGTGGTGTTTCTG | |
| | | | | | TTACCTCCCAGTGGTTACTTGCCTGCAGCTAGAAGG | |
| | | | | | GCTTTCTGCAGTGCTGCTGCTGGAGTTGGGGGGAAA | |
| | | | | | AGGCTGACACTCAGCACAGCCTTCTGCATCCACTTG | |
| | | | | | AGTCATGCAGGACACTTAGCTTTGTTCTTTCTCCAC | |
| | | | | | AGTTAATATTATGCCAAACCTACCTGTAATTAGTAA | |
| | | | | | TTTTCAAAGAATATTATAAGTTCCAGTAACCAAATG | |
| | | | | | TTTGGGCATAATTATATGCCAAAAGACTACTTTTTA | |
| | | | | | ATTGATAATTTTTAACTGCTTTTTATATATTTGCAGC | |
| | | | | | CTGAGAAGGCTGTTTGGATACTGAGGTTCAGCAAA | |
| | | | | | GTGGGTCTGAAGATACTTGTTTATGCAAATGGGACT | |
| | | | | | TTGTAACCTGGGAAATCTACAGGATTTATACAAATT | |
| | | | | | ATTATTGAAATAGGCTTAACTGTCCGGGCACGGCA | |
| | | | | | GCTCATGCCTGTAATCCTAGCACTTTGGGAGGCCAA | |
| | | | | | GGTGGATGGATTGCTTGAGCCCAGGAGTTCAAGAC | |
| | | | | | CAGCCTGGGCAACATGGTGAAACCCTGTCTCTACA | |
| | | | | | AAAAATACAAAAATTAGTCAGGCGTGATGGTGCAT | |
| | | | | | GCCTGTGGTTCCAGCTACTCTGGAGACTGAGGTGGG | |
| | | | | | AGGATCACTGGAGCCCAGGGAGTTAGGGCTGTAGT | |
| | | | | | GAGCCAAAATCATGCCACTGCACTCCAGCATGGGC | |
| | | | | | AACAGAGTAAGACTCTG | |
| 44 | 1336656 | | 44 | | GCTTTGAACAGCTTCCCCTTCCATCTGTAACTATTG | |
| | | | | | GGTGAGGTGGAATTATTTTAATTTGTTCTACATGC | |
| | | | | | TGACCAGTTGCCCCTCTGTTTACTGAATTATTATGTC | |
| | | | | | TTCTCCATGAGTTTGAAATGCCATTTAATTATATGT | |
| | | | | | TGTGTATGTGTATTTATACGTATATGTTTATCAGCTC | |
| | | | | | TCTGTCATTGATTTTTCTTCTTGCACACATAGTATAA | |
| | | | | | CATTTTAATTACTGTACCTTTATACCAGGTCTTGGC | |
| | | | | | AAACAATGGCCCATGGGCGAAATCCAGCCCTACCA | |
| | | | | | CCTGGTTTTTATAAATAAAGCTTTATTGGAAAGCAG | |
| | | | | | CCATACTTACGTATTGTTTATGGCTGCTTTTGAGCTA | |
| | | | | | CTATGGCAGTGTAGTTGCAACAGAGACTGTATGGG | |
| | | | | | CCAGAAATCCAGAAATATTTACAATCTGGCCCTTCA | |
| | | | | | TACAGAGTTTACCAGGCTCTGCTTTATACTGTGTAT | |
| | | | | | TGATATCTGATAGGGCAAGTTCATCCTCATTCTTTTT | |
| | | | | | CAACAATTTCTTGGCAGGCCTAACATGTTTATTTCT | |
| | | | | | CCAGATGAACTTTAGAATCAATCTGCCAAGCTTGCC | |
| | | | | | TGACTTCCTTCTTTTCCCCACCTCTTTTTGGGGTGGA | |
| | | | | | GAACTGGGGAGCCAGCAGAATAGGAATTTTGATTG | |
| | | | | | CATTAATTTGTGGTTTAGTGAAGGGAGAAGTGATTG | |
| | | | | | CTTTACACGTTGGGTCTTTCTATTCCAGAAATATC | |
| | | | | | TCTTTACGTGTATATCTTTCAGTAAACTTTAATTGTT | |
| | | | | | CATTCTGAACAATAAAATCATACATATTGGAGTTTA | |
| | | | | | TTCCTATATGTATTGTTGCTTTTTGTTGCCATTATAA | |
| | | | | | TTGCGTTCTTGTCCCAGTTATATTTTGCAAGTGACTA | |
| | | | | | TGGTATAAAGGGAAGTTTTTGCTTTTTATGTATTTA | |
| | | | | | AATTCTGTTTCTAACCCTCTTATGAGAGTAAACTAT | |
| | | | | | TAGGACTGTTAATTTTTGTTTCTTTTGATTGAGAGTC | |
| | | | | | ATTGTCTGAACTTACCAATAATTGTTTTATTAATGTT | |
| | | | | | TATGTCTCCCCCTGTATTGTGTAGTTTTCTTACCTAG | |
| | | | | | AGTAGTTTGGGGGAATGGACTTTGACCCCCTCAATG | |
| | | | | | GCATTCATTTTTTTTTCTTTTGTGTAGGTCACAGCAA | |
| | | | | | ATGGTAGTTAAAACAAGC | |
| 45 | 459363 | | 45 | | GGAAAGAAAATGTAGAAATAACAGAGATCAAACA | |
| | | | | | AAAAAACAAAAACGGCAGACTTAACCCTAACATAC | |
| | | | | | TACCAATAATGACATTAAATGGAAATTAAATGGAG | |
| | | | | | TACCAATCAAAAGAGGTGGTAGGGGTAGATTTTTTT | |
| | | | | | AAATCCCCCATTTATATATCTGTCAGAAACTCTTCA | |
| | | | | | AATATAACAATATAGGCAAGTTGAACATCGGAAGA | |
| | | | | | TGTGAAGAGATAACATAACAAATATTAAAAAGAAA | |
| | | | | | GCAGCATATTGGCAATGTTAATACCAATTAAAGTA | |
| | | | | | GACTTCAGAGCAAAGAAAATTACCATGAACATAGA | |
| | | | | | GGAATATTTACATAATGATAAGA | |
| 46 | 899587 | | 46 | | AATGATGGATCATTGGTGATAAATACACAAAAACC | |
| | | | | | CAACCAAACAAAGACAGTTACTCCAGGAATAACAA | |
| | | | | | AAATGTGTGCAGGAAAGGAAAAGGATTCCAAGTAC | |
| | | | | | ACAAGGAACTCAGCTGCCCCTATAGCACTTAGAAA | |
| | | | | | GTCATGATAAAGTCAACAGTGAACACAGAGTTAAA | |
| | | | | | ACTCTGTGGGGACAGGGGAAAATATTTGTCATGGG | |
| | | | | | AAGTGAGGGGATATTTGAGTAAGTGAATGTTGGAT | |
| | | | | | CTTTATCTTCCATAATGGCAGGTTCATAACAATGGC | |
| | | | | | TACAAACTATAGCAGTTAAAAGAATTAGCCGGGGC | |
| | | | | | CCGGTGTGGTGGCTTACACCCATAATCTTAGCACTC | |
| | | | | | TAGGAGGCCAAGGCAGGCAGATCACTCGAGGTCCG | |
| | | | | | GAGTTCAAGACCAGCCTGGCCAACATGGTGAAACC | |
| | | | | | TGTCTCTACTAAAAATAC | |
| 46 | 899587 | | 22 | | AGAATGATGGATCATTGGTGATAAATACACAAAAA | |
| | | | | | CCCAACCAAACAAAGACAGTACTTCCAGGAATAAC | |
| | | | | | AAAAATGTGTGCAGGAAAGGAAAAGGATTCCAAGT | |
| | | | | | ACACAAGGAACTCAGCTGCCCCTATAGCACTTAGA | |
| | | | | | AAGTCATGATAAAGTCAACAGTGAACACAGAGTTA | |
| | | | | | AAACTCTGTGGGGACAGGGGAAAATATTTGTCATG | |
| | | | | | GGAAGTGAGGGGATATTTGAGTAAGTGAATGTTGG | |
| | | | | | ATCTTTATCTTCCATAATGGCAGGTTCATAACAATG | |
| | | | | | GCTACAAACTATAGCAGTTAAAAGAATTAGCCGGG | |
| | | | | | GCCCGGTGTGGTGGCTTACACCCATAATCTTAGCAC | |
| | | | | | TCTAGGAGGCCAAGGCAGGCAGATCACTCGAGGTC | |
| | | | | | CGGAGTTCAAGACCAGCCTGGCCAACATGGTGAAA | |
| | | | | | CCTGTCTCTACTAAAAATACAAAAAAAAAAAAGAC | |
| | | | | | GAG | |
| 47 | 334519 | | 47 | | AACCCCTCTCCCCAGAGGATGCCTTGCCTGGTGAGG | |
| | | | | | TCAAAGTACAAGATGGTGCCAGTTACTGAGATTTG | |
| | | | | | GCCGAAATGGTCTTGGGGTAGTCGCGGGAGTTTGG | |
| | | | | | AAGTGGGGGTAAGGTTGCTGGAAGGTTTCAAGGTC | |
| | | | | | TCTCATCTGCTCCCTCTCCGTTTCCCATGAAATGCCC | |
| | | | | | TTGTTTAACGGGCTGTGGTGCCGAACTCCGGGATCA | |
| | | | | | CTCCCACAGCCTGGAAGGGAGCCGTTGCCTCCAGCT | |
| | | | | | GCAGTGCATCAAGGGAGCTCGGAATAGACCCTGCC | |
| | | | | | CTCTGTCAGCTGCACCAGTGGCTGTCCATGGGGGGA | |
| | | | | | GAGGCAGAAGCCTACCAGAATTTCCTGTCTTGGCTC | |
| | | | | | CCCAGATCAGAATCAAGGGACTTCTGGCCTCTGGA | |
| | | | | | CTGAGGAAGTGACATTCTGTTTTTTCAAAGGAAGTGT | |
| | | | | | TGTTGTTGCGGAGTACAAGTGTGTGTCAATGAAATC | |
| | | | | | AGGCTCTTAGGTAGATGTTTGCTGGGGGAAAAAAA | |
| | | | | | TCTAAGGATTTAGCACATGAGTTTTGAAGTGGACG | |
| | | | | | TGGATTTATAGGAGGAATGAAGCAGTGAATTGTTC | |
| | | | | | ATCTCAGTTCGGAAGCTCATTTTTAGGAGTGTCTAT | |
| | | | | | GTAGCCAAAGTATAATTATTAAGAAATAAACTTTTT | |
| | | | | | TCCTCTTTCAGGGTTGTATCAGTTCGTTAGGAAGGT | |
| | | | | | GAATATTTTAATTAGGATTAAGGAGCAGTGATTTAC | |
| | | | | | TATTAACAATTTTATAAATAATTTAAAAACTTTGTC | |
| | | | | | CCGAAGAGCTTCCAAAAATTATCTATACAAATAGA | |
| | | | | | TTTCCATACAAGCTAGTGGAATACAGTGTCCACAGT | |
| | | | | | AAAAAAAAAAAAAAAAAAAAGTGACCCTTAATTTTC | |
| | | | | | AAGTTTGAACACTATACACTAAAGAACCTTGAAAG | |
| | | | | | TTGTTTTTGAAACAATTTGCAAACAGTATGACACTG | |
| | | | | | TATCTACATTTGACTTATCGCTCCTTGAACTCTCACC | |
| | | | | | CAGACTCTATGACCCATTTCTTGGGTGTTTTTGTTCC | |
| | | | | | CAAACAACTTTAGTTCAAAATAACCAGGTTTGGAG | |
| | | | | | GCATTGGGTCAAGCACCTTTTTCACTGATTTGAAC | |
| | | | | | GAATCTAGTCGTATGATGGCCTTAGC | |
| 48 | 185587 | | 48 | | GTGCAATGGCACAATCTTGACTCACCACAACCTCCG | |
| | | | | | CCTCCCGGGTTTAAGCGATTCTCCTGCCTCAGCCTC | |
| | | | | | CCAAGAAGCTGGGATTACAGGTGCACGCCACCACG | |
| | | | | | CCCAGCTAATTTTGTATTTTTAGCACAGACGGGGTT | |
| | | | | | TCTCCATGTTGGTCAGGCTGGTCTCAAACTCCTGAC | |
| | | | | | CTCAGGTGATCCGCCCACCTTGGGCTCCCAAAATGC | |
| | | | | | TGGGATTACAGGCATGAGCCACCGCACCCGGCTGG | |
| | | | | | GGTTTCTTTGTATCTTTTATTTATTGAACCTTTGTTTT | |
| | | | | | TGGAGTGTTCATAGTTTCTTGTTAAAAGTGTTTTTGT | |
| | | | | | TTGTTTTTTAATGATAGCTGCTTTAAAAATCCTTGCC | |
| | | | | | AGACAATCCCAACATCAGTACCATCTTGGTACTGGC | |
| | | | | | ATCTGTTGATTGCCTGTTCTCATTCTGGTTGACTTTT | |
| | | | | | TCTGTTTTCTGACATGACAAGTAATATTCAATATTA | |
| | | | | | TCAGTACTTTGGGTATTATGAAACTCTGATTCCTTTT | |
| | | | | | TATATTTTCTACTTTAGCATGCATTCAACCTGCTTCA | |
| | | | | | ATTCAGAATGCACATCATGACTCACTTCTGTGGTCT | |
| | | | | | GTGAGTTTGAATGTCAGTTTGGTTTCAAATTCAGCG | |
| | | | | | TTATCTTGGTCTGCTCTGCCTGTGTGCTACCCAGAG | |
| | | | | | ACCAGTGGATACCCAGAAACCCGAGTGGTATTCCA | |
| | | | | | CAGCATAGCTCAGTTCTTAAAGCTTTTGCTGTGTTA | |
| | | | | | ATTCTGATGAGTTTCACACATAGGCCACTTGGGGAT | |
| | | | | | GTGCACAAATTGAAAGACGCTTTTTCCGCAGCTCCC | |
| | | | | | TCCTCTCTGTTATTCTGCCCACACTCTCTGTGAGGG | |
| | | | | | GGTAGGTGCTGCCTCTGTTACTGCAGGACAGGTGGT | |
| | | | | | AGTCAACAGGGCTCTACCCTAGAGTGTCCATAGCAT | |
| | | | | | CCCATGGGAAGAAGGAGGAGGGAGGGGGTGTCAC | |
| | | | | | CTCTTATCCCATTAGTGCAGGATGGGGCTCATTAAT | |
| | | | | | AGAGCTCCACTTGTCTCCAGAATCACTGGTGAGGA | |
| | | | | | AGGGGAGTGTTGCCCCCACATTCGTGCACAGCAGG | |
| | | | | | GATGGTTCACCGAACTCCACACCAGTCTCTGCAGAG | |
| | | | | | CCTGTTGGGGAGAGGAGGGCTGTGGTTTCTTTGATG | |
| | | | | | GTGTTCACCTGGAGTAGAGCAAGTATTGTCAAAAG | |
| | | | | | GGTCATCCTCGGAGGTTGCAGTGAGCCGAGATCGC | |
| | | | | | ACCATTGCACTGCAGCCTGGGAGACAGAGCAAGAC | |
| | | | | | TCCATCTCAAAAAAAAAAAAAAAAAAGGCCATCCT | |
| | | | | | TCATTACTGTCCTCTTCTAGGTCCTTTGACTAGAGA | |
| | | | | | AAGCATTTTCCTTAGGACTTTTGTTGTCTGTGCTTGT | |
| | | | | | TGGTCATTTCAGATTGTGCCTTCCTCTAGTGCCCAG | |
| | | | | | GTTGAAATACGTGAACACTACGAAAGCCTCTGGGA | |
| | | | | | ACTCCCTGCCAGGTCATCCCTGAGACTTAAGTTC | |
| | | | | | CTTGCCAGTCTGCCTAGTTTACTTCACCTTTAGAGG | |
| | | | | | TTTCT | |
| 49 | 436375 | | 49 | | CTTAAAATGATACCACTTCATAGTTAATACCAGCAA | |
| | | | | | ACTGCTTAGTCAAACCATACTATGCGGCCCTCCACC | |
| | | | | | CAAGAGCATTGTTTGTGCAGGTAGGATCTGCAGTGT | |
| | | | | | GGATGGAGGGTAATGGAAAATTGTGGCCACTGTTA | |
| | | | | | GCTGGTCAGACTGATATTTACTGTATGTCAGGTACT | |
| | | | | | GTGCTGAGTCCTTCATGGGTATCATTTCGTTTGGTC | |
| | | | | | CTTGCAATAACCCTATAGGGCAGGTCCTATTATTAG | |
| | | | | | ATGCATTTTTTAGCTGGAGGTGATCACACTGCTGAA | |
| | | | | | AAGTGACAACCAGATTCAAATGCAGAGTTTCTGAC | |
| | | | | | TACTGTGATATAGGGTCCCGGATGGCACGTGCTAC | |
| | | | | | CAGCAGCCAAAGAGAGTCCATTTGGCCTTGGAATTT | |
| | | | | | CTAACTCAGAGACTGAAACACAGAGGGACTTGTAG | |
| | | | | | GTGGAACCAAGGTTTAAATGACTTAATTGGATGGG | |
| | | | | | CTTACCTTTGGAGGAACACCATAGAAGCAAATGTG | |
| | | | | | TTTTTCAAAGATCTTCCACTAGATGTCACCAAAAGG | |
| | | | | | ACTGAGAACTAGAGAAAAGGGCTGCGATTTCTGCT | |
| | | | | | CTCCTGTTAAGATTGCACAAAAGAATAAATGCATT | |
| | | | | | TATCGCTGTTTGC | |
| 50 | 337323 | | 50 | | ACATTGTATGTGTGCCTCTAGGAGGGTCACTGAGAT | |
| | | | | | TTATGATAAACATATATATTGATTGTCCAAGAAAAG | |
| | | | | | GTGAAGAAACATTAACCATAAGTCACAATTCCATG | |
| | | | | | AACACATTTAAAAGTAATTAGTAAATGTGCAGAGA | |
| | | | | | CACTGTTAGGGGAGTGGATGTTACTACTGTCATTTA | |
| | | | | | TGAAGGATTTGCTAGAGATGGTAGATTTCACCTGTT | |
| | | | | | GTGAATTGGAGGAGGAGCATGGCTGGCAATTCGAA | |
| | | | | | AGGAGGTAATCTCTCTGGGGTACAATGGAGTAGAA | |
| | | | | | AACTTAGGGACAGAAGGAATATACGAATGGAGAAA | |
| | | | | | TTCGATTTGCCCAATCTTTATTGCTCACCTATTAAAG | |
| | | | | | TGCTAAACAAGCTGATGGTGATTCCTGTTCTCAGAA | |
| | | | | | GCCTGTGTTCTAGCAGGTTATAAGAAGATGAGTCTG | |
| | | | | | GTTAAAGAGAAGAGCAGGGAAGTGGCTTAGATTAT | |
| | | | | | GGCATAAACTGAAGTTGAAACTCAGAATGAAAAGT | |
| | | | | | AGGAGTTTGCTGAGGGGAAAGCAATATATAAAGTG | |
| | | | | | ATTTGTGCTATAGGACTAAGACAGATTATAGATA | |
| | | | | | AGAGAACTCAGAAATAGTAAGGACAGTGGTAAAAA | |
| | | | | | GTTAAAGGATCCTCCCTTTCCCCAGTTAACCAGGAG | |
| | | | | | ACCAAATAAGGGACTTGGTGGTAGGAGTGGTAGGA | |
| | | | | | GCAGGATCAATCACTTATTATTAAGCACCTGCACA | |
| | | | | | TGATTCAAAGAAGGATAAGACGGCCCCTACCCTTA | |
| | | | | | AGGAGTTTATGTTCTTTCTAGTTGTGAATAGAGAAA | |
| | | | | | GCATATGC | |
| 50 | 337323 | | 273 | | CCTGTGACATTTCTTTCAGGAAGTCTTACACCTTAC | |
| | | | | | TTGACTCCACAGTCTAATTAGATGTTACCTCCTTGG | |
| | | | | | GATCCCACAGTAGTGTATGTGCCTCTTTCACAGCAA | |
| | | | | | TGCTGTCTATACTGACCCCTGATTTGCATGTATACC | |
| | | | | | TTTCCTCAGGATATGAACTTTCTGAATGTAGTGACC | |
| | | | | | ATACCCTATTTATTTTGATATCGCCAAATTCTAGCTT | |
| | | | | | GTGCTTGACATAGAGTTGTTTCCCAGCTAAATGTTG | |
| | | | | | AATAAAAAAACCAAACTGAAAAAAACATAGGTAGCAT | |
| | | | | | TATGTGTAATATTTACTATATAGGTTCTGATTTAAA | |
| | | | | | TGCTTTACATATATTAACTTATTTAATCATCATAGC | |
| | | | | | AACACTATGGGGTAAGTACTATTATTCCTGCCTCCA | |
| | | | | | TTTTACAGGTGAGGAAACTGAGGCTTGCAGAGATT | |
| | | | | | AAATAACTCTCCCAAAGCCACACAGCTAGTAAGTG | |
| | | | | | GTGGAGCTAGGATTCAAACCCAGGTAGTGTGGCTT | |
| | | | | | CACAGTTAGTGCTTTAACCACTACATTGTATGTGTG | |
| | | | | | CCTCTAGGAGGGTCACTGAGATTTATGATAAACATA | |
| | | | | | TATATTGATTGTCCAAGAAAAGGTGAAGAAACATT | |
| | | | | | AACCATAAGTCACAATTCCATGAACACATTTAAAA | |
| | | | | | GTAATTAGTAAATGTGCAGAGACACTGTTAGGGGA | |
| | | | | | GTGGATGTTACTACTGTCATTTATGAAGGATTTGCT | |
| | | | | | AGAGATGGTAGATTTCACCTGTTGTGAATTGGAGG | |
| | | | | | AGGAGCATGGCTGGCAATTCGAAAGGAGGTAATCT | |
| | | | | | CTCTGGGGTACAATGGAGTAGAAAACTTAGGGACA | |
| | | | | | GAAGGAATATACGAATGGAGAAATTCGATTTGCCC | |
| | | | | | AATCTTTATTGCTCACCTATTAAAGTGCTAAACAAG | |
| | | | | | CTGATGGTGATTCCTGTTCTCAGAAGCCTGTGTTCT | |
| | | | | | AGCAGGTTATAAGAAGATGAGTCTGGTTAAAGAGA | |
| | | | | | AGAGCAGGGAAGTGGCTTAGATTATGGCATAAACT | |
| | | | | | GAAGTTGAAACTCAGAATGAAAAGTAGGAGTTTGC | |
| | | | | | TGAGGGGAAAGCAATATATAAAGTGATTTGTGCTA | |
| | | | | | TAGGACATAAGACAGATTATAGATAAGAGAACTCA | |
| | | | | | GAAATAGTAAGGACAGTGGTAAAAAGTTAAAGGAT | |
| | | | | | CCTCCCTTTCCCCAGTTAACCAGGAGACCAAATAAG | |
| | | | | | GGACTTGGTGGTAGGAGTGGTAGGAGCAGGATCAA | |
| | | | | | TCACTTATTTATTAAGCACCTGCACATGATTCAAAG | |
| | | | | | AAGGATAAGACGGCCCCTACCCTTAAGGAGTTTAT | |
| | | | | | GTTCTTTCTAGTTGTGAATAGAGAAAGCATATGCAA | |
| | | | | | AAAAAAAAAAAAAAAAAGGAC | |
| 51 | 251758 | | 51 | | GAGACGGAGTTTCGCTCTTATCGTCCAGGCTGGAGT | |
| | | | | | GAGTGTAGTGGCTTGATCTCAGCTCACTGCAACCTT | |
| | | | | | TGCCTCCCGGGCTCAAGCGATTCTCCTGCCTCAGCC | |
| | | | | | TCCCAAGTAGCTGGGATTACAAGCATGTGCCACCAT | |
| | | | | | GCCCAGCTAATTTTCTGTATTTTTAGTAGAGACGGG | |
| | | | | | GTTTCACCATGTTGGCCAGGCTGGTCTCGAACTCCT | |
| | | | | | GACCTCAAGTGATCTGCCCGCCTCAGCTTCCCAAAA | |
| | | | | | TGCTGGAATTACAGGCATGAGCCATCACGCCTAGC | |
| | | | | | CTACTCTCTGAATTTCTAAAAGTCAGTAGGTTGACC | |
| | | | | | AAAAAGTCTAGAAACTGGCTTTAAGTCAGTATGGG | |
| | | | | | ACGTACTTATAAAGAGTCCATGGTTTTGCACGTTTC | |
| | | | | | GGTAGACAAGTAAATCTGAGTTATTTTTCAATGACT | |
| | | | | | TACCAATATTTGAATAGTAACTAAGATCGTCAGTGT | |
| | | | | | ATCTGGACTTCTTTTTTTGAAGTTCTAAACAATTAT | |
| | | | | | AGTAGGGATTTATTATTTTGGGCCTCCATCCAGATG | |
| | | | | | TTTTTCCAAGATCATTTTTAAAATTCATTTGTCTTCT | |
| | | | | | GTTTCCAGATAACATACTTTCCGTTCTATAGGAATC | |
| | | | | | TTCACTGCCAATCATAGTATCTACCAGTGGCTTTCT | |
| | | | | | TAGACTATTCACTCCAAAGCTGGGACTGATGTCCTG | |
| | | | | | CCAGTAGAGAATCTACAGAAATAATTTGAATGAAT | |
| | | | | | TAAAACCAAATCTTGATAGCAGGAGACAGCTTCCT | |
| | | | | | GATCTAGATGTACAATTAGAGTTTAGGTTGGAAATT | |
| | | | | | ACTTTAAAATGTGTTTTTTTGGGGATGTCTTCAATCTC | |
| | | | | | TGTGTAAATACCCACATGCTTATGCATTGTAAACCA | |
| | | | | | AGTGTGTATTCCTGTGTATGAATTTGTAGAACTGAT | |
| | | | | | TTCTGCTTCAAGAGAAGCTGCACCTTTAATTTTATA | |
| | | | | | AGGTCCCCTCCACCTGTAACCCTATAAATGTCTGTA | |
| | | | | | AATAAAACACTAAAATTTGTAGTGATAGGATCAAT | |
| | | | | | TTGGGAATATCTGCTGAGAGACCAAAAAAGTTCATTT | |
| | | | | | TTTTAAGTACCTTGGTTAAAGAGTAAAGATTATTCC | |
| | | | | | TCTTATTTTTTAAAGAAGAATGCACTTTAACAAACA | |
| | | | | | TAGAGCTGCATGGGCAATTCAAACAAATCTGTGAA | |
| | | | | | GTGCAGTACCCATTCAGAAATCACACTTCCTGAAAA | |
| | | | | | CCGTTCAAAAGCAGAGTCCAGACGGGCTGTTGATC | |
| | | | | | TCACTGCCTGTAGGTTGAAGCTCAGATTCTGATCCAA | |
| | | | | | TTTTGAGAGGAGCAGGGCTGCTTCAAAAGAGCAAT | |
| | | | | | GTGAATACAGTCAGAAGCTTCAGACTGGTCTGTAA | |
| | | | | | AAATGGCGGGTCCCGTATTTACCACTAACTAGCAA | |
| | | | | | AACTGACAGAAAAACTCACAGAGAAAAAATGTAAG | |
| | | | | | AATCCTTCCTGCTGGTGTGCACTCCTTACAATAGAC | |
| | | | | | TTTTGCAAATGGAGTTTTACAGTCTATATTTAAAAA | |
| | | | | | AAATTGTATGTTTGTAACAAATAAAGTATGCAGAA | |
| | | | | | AAGTGAATGACAATCTTGTGCTTGTGT | |
| 52 | 346607 | | 52 | | GAATCGATTGAAATAGTATATGAAGTGGTTTGAAA | |
| | | | | | ATAGGTACAAACTATTGACATTTCAATGTCAGAGA | |
| | | | | | GTGATACCTGTAGTAGTATAGGCAAAGGTCCAACC | |
| | | | | | CCATCGAAAGGCTTAAACATTTACCTTTTCTGAAAA | |
| | | | | | ACTATTGAAATATAAAGAGAGTCCCCAGTCACAGG | |
| | | | | | GGCAACTTCTGTAACCAAATCCAGATCTGAGGAAA | |
| | | | | | CTCCTGTAACCCCATTTGGGGTTTCTTTCTAAGCCA | |
| | | | | | ATAGGGTTACAGGTTGGTACAGTGACACATTGAGA | |
| | | | | | ATGGGGCTACAAATACTTTTCCCACCATCTAGGATG | |
| | | | | | AAATACACGAAATCCTGTTGAAATCTTGGTTTTTAT | |
| | | | | | GCCTTTGCTCATCAGAATAAACGTAAATGCTGAAA | |
| | | | | | AACAAATAACCTCCTGATCCACTGTCTTGCCTCCTG | |
| | | | | | GTGAGAAATGATTCTATCCCCTGTTTATTGGGAAAT | |
| | | | | | TTCCAAAGTTGTTCATCACTTAAATGCCGTATTCAA | |
| | | | | | AGGGAACATGGAAGGATGAAGCGGAGAAAGTGCC | |
| | | | | | TTCGAGACATTCACACATTTCTCTGGACTCAGTCTG | |
| | | | | | TTAACATATCAGGGAGCTTGTCAGATCACACCTTTT | |
| | | | | | TGCCTTGGAAATCCTACAGATTTCCTGTACGCCTTC | |
| | | | | | ATATCTGATTCTTCCCTAAAACCTTTGGGTATGATTT | |
| | | | | | CCTCCCTGGTCTTGATAATGTCCTGCAGTCTGTGTTT | |
| | | | | | TATAATTATTCTTTGTATTTATTGAATCTAGACTTTA | |
| | | | | | AGTTATTCAGAGATCAGACCAGAACCTTAGAGTTTC | |
| | | | | | TAAACTGTATGTGGATATTAAATAATATTAATAATG | |
| | | | | | AAAGAGCTACCAAAATAGTCTATATTGTGTGAACA | |
| | | | | | ATCTCTTGGGATATTAGACGTGTTTAAAGACCAGTG | |
| | | | | | TTGCTGCTATTTTTAATATTTTGGTTAATTTAAGTGA | |
| | | | | | AATGTACATATTTTAATTTGAAGATTTATCTTGCCC | |
| | | | | | ATCAGAATGTGAAGATATACTTGCATATATTTTGAC | |
| | | | | | ATATTTCATGGAAAATAAAAATGATAATCCACTTTG | |
| | | | | | TGAGTGTAAGTGAATGTATTCATATGTATGTTATTA | |
| | | | | | TAAATGATTTTTGTTTGCACTGATGATGAAATGAGA | |
| | | | | | GTTTTGGGGGTTTTTATACATTTATATCGACTGGTC | |
| | | | | | TCTAAATCTCCTATTTTGTTTTCTTATCATTTTTGAA | |
| | | | | | ATACAGTTCCCATTACATGAGTTTAAATAGATTGG | |
| | | | | | TGTTTCATTTTGTATTATGCTACTACTAGATGTTGAT | |
| | | | | | TCTCTGGTATTGTAAAATAAATGTGCTCCAAAAAC | |
| | | | | | CCAAAAAAAAAAAAAAA | |
| 52 | 346607 | | 274 | | GCCAAGTTCTGCAAAAGATCCATACCAGTTCACTCG | |
| | | | | | TGTGCGACTGTGGACAGGTAAGTCACTTTGGTCTCT | |
| | | | | | ATGAACCTCAGTTTTCCAGATCTTTGAAATGAGCAC | |
| | | | | | TTGGATGCCTATCCTTGCTTCCACACAAGTGTTTTT | |
| | | | | | TTTTTTTTTTTTTTTTTTTGTGAGAATCGATTGAAAT | |
| | | | | | AGTATATGAAGTGGTTTGAAAATAGGTACAAACTA | |
| | | | | | TTGACATTTCAATGTCAGAGAGTGATACCTGTAGTA | |
| | | | | | GTATAGGCAAAGGTCCAACCCCATCGAAAGGCTTA | |
| | | | | | AACATTTACCTTTTCTGAAAAACTATTGAAATATAA | |
| | | | | | AGAGAGTCCCCAGTCACAGGGGCAACTTCTGTAAC | |
| | | | | | CAAATCCAGATCTGAGGAAACTCCTGTAACCCCATT | |
| | | | | | TGGGGTTTCTTTCTAAGCCAATAGGGTTACAGGTTG | |
| | | | | | GTACAGTGACACATTGAGAATGGGGCTACAAATAC | |
| | | | | | TTTTCCCACCATCTAGGATGAAATACACGAAATCCT | |
| | | | | | GTTGAAATCTGGTTTTTTATGCCTTTGCTCATCAGA | |
| | | | | | ATAAACGTAAATGCTGAAAAACAAATAACCTCCTG | |
| | | | | | ATCCACTGTCTTGCCTCCTGGTGAGAAATGATTCTA | |
| | | | | | TCCCCTGTTTATTGGGAAATTTCCAAAGTTGTTCAT | |
| | | | | | CACTTAAATGCCGTATTCAAAGGGAACATGGAAGG | |
| | | | | | ATGAAGCGGAGAAAGTGCCTTCGAGACATTCACAC | |
| | | | | | ATTTCTCTGGACTCAGTCTGTTAACATATCAGGGAG | |
| | | | | | CTTGTCAGATCACACCTTTTGCCTTGGAAATCCTA | |
| | | | | | CAGATTTCCTGTACGCCTTCATATCTGATTCTTCCCT | |
| | | | | | AAAACCTTTGGGTATGATTTCCTCCCTGGTCTTGAT | |
| | | | | | AATGTCCTGCAGTCTGTGTTTTATAATTATTCTTTGT | |
| | | | | | ATTTATTGAATCTAGACTTTAAGTTATTCAGAGATC | |
| | | | | | AGACCAGAACCTTAGAGTTTCTAAACTGTATGTGGA | |
| | | | | | TATTAAATAATATTAATAATGAAAGAGCTACCAAA | |
| | | | | | ATAGTCTATATTGTGTGAACAATCTCTTGGGATATT | |
| | | | | | AGACGTGTTTAAAGACCAGTGTTGCTGCTATTTTTA | |
| | | | | | ATATTTTGGTTATTTAAGTGAAATGTACATATTTT | |
| | | | | | AATTTGAAGATTTATCTTGCCCATCAGAATGTGAAG | |
| | | | | | ATATACTTGCATATATTTTGACATATTTCATGGAAA | |
| | | | | | ATAAAAATGATAATCCACTTTGTGAGTGTAAGTGA | |
| | | | | | ATGTATTCATATGTATGTTATTATAAATGATTTTTGT | |
| | | | | | TTGCACTGATGATGAAATGAGAGTTTTGGGGGCTTT | |
| | | | | | TTATACATTTATATCGACTGGTCTCTAAATCTCCTAT | |
| | | | | | TTTGTTTTCTTATCATTTTTGAAATACAGTTCCCATT | |
| | | | | | ACATGAGTTTTAAATAGATTGGTGTTTCATTTTGTA | |
| | | | | | TTATGCTACTACTAGATGTTGATTCTCTGGTATTGTA | |
| | | | | | AAATAAAATGTGCTCCAAAAACCCAAA | |
| 53 | 402834 | | 53 | | AGAAACTTCACTGCTATTTCCAGATGTCATTTTAAA | |
| | | | | | ATATTTTAGAATACCTGATTTCTCCATGACCTATCC | |
| | | | | | ATGCTTTTCTAAGGTTCCAAACTAAAATGCAGAATC | |
| | | | | | TTGAGTTATTCCAGAACATAGATTTAAAATTTGATC | |
| | | | | | AGAAAATAACCTTACATTTAAGAAATGAGGGGTCAG | |
| | | | | | GCGTGAGCCACCACGCCTGGCCACCAATTTTTATTA | |
| | | | | | TATGATTTTATAACTAAAATTTCATAACTAGCTAAT | |
| | | | | | GAAATTCTTCTTCTCTCTTTTTTGTTTATTTATCTTCC | |
| | | | | | TTTTAGTCTTTCTTTCTCCTCGGATCTTTCCCCTTCTA | |
| | | | | | TCTGTCTCAGTTCCTTCATTTTCCTTAGCTCTCCATT | |
| | | | | | TCTCCCAGCATCTGCTACTAGTCTAGTCTCCTGGCT | |
| | | | | | CTTAACCTTTTTGAGACACAGACTCCTTTAATAAAG | |
| | | | | | TGATGAAGAAAGTTATCTCCCCAGAAGAATACACA | |
| | | | | | CAGAGAACACAGAATATTTTGCGTATATTTCAAAG | |
| | | | | | GTAAAGAATGCCAAGAAGCCAGGGGCAGTAGTTCA | |
| | | | | | TGCCTGTGATCCCAGTGCTTTCGGAGGCTGAGGTGG | |
| | | | | | AAGAATCACTTGAGCCCAGGAGTTCGAGGCTGGCC | |
| | | | | | TGGGCAACATGGTGAGACCTCCTCTCTACAAAAAA | |
| | | | | | ATTTTAAAATTAGCCAGGTGTGCTGGCACGTGCCTG | |
| | | | | | TGGTCCCAGCTACTCAGGAGGCTGAGGTGGGTGGA | |
| | | | | | TTGCTTGAGCTCAGGAGGTGAAGGCTGCAGTGAGC | |
| | | | | | CATGAGTGCCACTGCACTTTCAGCCTGGGTGACAG | |
| | | | | | AATGAGACCCTAGCTCTAAAAAACAAAGGATGCCA | |
| | | | | | AGTATCTAAACTTTGAGCTCCTGAGGACAAAAACT | |
| | | | | | AGGCGTTTTTCATCCTATATGCCCAGTATTTAGTTG | |
| | | | | | ATGTTTCTTGAGTGTATATAAGTGTGCACATGCCCA | |
| | | | | | GAAACATGTAAATATTAGTACATGTTGTAGAAAAG | |
| | | | | | CTGTTGTCAGGAAGATATTTGTACACTCTGGCTTTC | |
| | | | | | CACTATGATAGTCACCAGGCACATGTGGGTACTGA | |
| | | | | | GCACTGGAAATGTGGATTGTCCAGATTGGAATGTA | |
| | | | | | CTAATTGTAAAATACGCACTGGATTGCACAGGCTTG | |
| | | | | | GGGCAGTACAAACAAAAGAATGAAGATATCTCATT | |
| | | | | | AATAGTTTTTATGATTATTACACATTAAAATGATCA | |
| | | | | | TATCTTGGATATATGAGTTAAAATATATTATTAAA | |
| | | | | | TTAATTTTACCTCTTTATTGTTACTTTTCTAAAAGCA | |
| | | | | | GCTACTAGAAAATTTTAAATTATACATGTAACTGCT | |
| | | | | | CATAGAAGGTTGGTATCTGGGTTCATTCATTAGTGG | |
| | | | | | ACATTCATAAACATAGTAATTTTCTTTAATTTCATG | |
| | | | | | GATTCGTTGAACTAAAGATCCCATAGGTCACCGCCT | |
| | | | | | TCCCTGTCCCTCCTCTACCACCAAAAACTTAATGAG | |
| | | | | | AACAAATGGGAAGAATTTACTCTGCTTTTCAAGGTA | |
| | | | | | CTCTGATACAGATTTTTATCTACTGTCATAAGTATA | |
| | | | | | CCTAGAACAAAAGCACTGTTGACTCAAGTAGTTTCA | |
| | | | | | CTAATGAAAAGGAAGCAGCAGAATGACTAATGTAA | |
| | | | | | ATTGGAGGAGACTCTTTTATTTGGAATGCTTTGGTT | |
| | | | | | CTTCCACTGTGGAACAGGTGTGGCTGCTGTTGAAAC | |
| | | | | | AGCAGAGTCATACTAGGCATATCTGACATGTGAGG | |
| | | | | | AACCGCAGCATTGCTCAGGGGCCCCTGCCTTCCAAT | |
| | | | | | GAATGGATGTAGGATCCATCATACATCAGATTGCTC | |
| | | | | | CTTTCCAATACAAACTCTGATGCAGAAATGCACTTG | |
| | | | | | GTGTATTTGCTTTTTCTTACTTTCTGGTTTAGGGCAG | |
| | | | | | AAATAATATTTTGGCTTGGAGACTTTTGTCCTGAAC | |
| | | | | | TATGACATAATAGGATGAGAATATCGTGTCAAAAA | |
| | | | | | TAGCCTTACAAGGTCCTTTTTGGCATTAAGACTTCT | |
| | | | | | GGAGTGAGTTTGCAGTGGATTATTGAGAATAATTCT | |
| | | | | | GTTCATTAGCAGCTAGCCATCTTTGATGAGTGCTGA | |
| | | | | | CTTCTCTCCTTTCAGCACAGAGCAGGAAATGCCTGC | |
| | | | | | CTCCCATGACTCTGGGTTGGAGTGAAGGGGAATGC | |
| | | | | | ATACCAGCCACCCTCTTGCAGAGGTGGGGCAGGTG | |
| | | | | | CTGGCACAGAGCCTCAGGTTAGGCCGAGGGGATGC | |
| | | | | | AATCTCAGATCAGCAGCCAGCAGTGTTTGTAAACA | |
| | | | | | ACAGGAGGGAGATTGTGCTGGTGATGTCCAACTCA | |
| | | | | | CACCAATGAAGATCAACCGGTTTGTGCTTTGGGCAG | |
| | | | | | CAGGCTGCAGATGGACAGTGCCTCCTGAGGGCATC | |
| | | | | | GCCATGTTTTAGGGATCCGTGTTGCAGGATACCTGT | |
| | | | | | CTGCAAGAGAGAGTCAAGGAGGGCTTTTTAAGCCC | |
| | | | | | CTGGGGTTCAGGCCTGGCATCTGGGTGTTAAGTAGA | |
| | | | | | GTGAATCTCCTGAAGTCCAAACTAACATATGACATT | |
| | | | | | TTAAAATGAGGAAAACAAATGGCTCTGAAAAGGTC | |
| | | | | | TATAGGATTATAGGTAAGTGGTTAATACGGAAGAT | |
| | | | | | GTTATAAAGGTCTCAGGAGGAGATGGGGTGATCCA | |
| 54 | 328027 | | 54 | | AAAATTGTCAATGTGGATGATTCTTTAAACCATAAT | |
| | | | | | TTGGGCCAAAAGCTGAGCATCACACCAAGAAAATA | |
| | | | | | TCTCTGCTTCTAGACATCAAGAAAGAGAGGTGGAG | |
| | | | | | ATAAAGGAAAAAACTTAATCCCGAATTGATAGGAG | |
| | | | | | TGAGAGACAACAAACCTTAGGACAGGGAATTCTTA | |
| | | | | | ACTTGTGGCAGAGCAAACAGTAGAAACTCATGAGA | |
| | | | | | CGTGTTATCCAATAATAGAAAATAGGAACATGAGA | |
| | | | | | TTTATCCACTAGACAGTACTAGGACTCTACATGTA | |
| | | | | | AACTCATGGGAATTGAAATAAAGTTCTCTGCTGTAA | |
| | | | | | TTGGAGCAAGATAGACTGAGGAGAGAGTAAACCAC | |
| | | | | | GAATGCTGGCTCAAGACAAAAAACCTAGCAGAGGT | |
| | | | | | GCATTGCAGACATACCCATGAAGGAAAAACTTACA | |
| | | | | | CAAGGTCACCCTAAAGGAAGGACATTGTTAAGCCC | |
| | | | | | TTTGAAATAATGGGGTGGAGAGGAAAATGAACTGA | |
| | | | | | AAAAATGAAAAACACCCACAGGAAGAAATCAAAG | |
| | | | | | ACGATTGTGTCAACCCCAGGGCTACAGAAGTGAGG | |
| | | | | | AATAAAATTGGCTATTTCCGGACACTGACTTTCTTG | |
| | | | | | ATTTTGTTGAACATACGTGAAAGCAGGACATGCCAT | |
| | | | | | GGTCGCTGGTTGCATCAAATAGAAATGACTCATTGG | |
| | | | | | AATGTTACCTCCAAATCCTTACATGAAGAGTAAGCA | |
| | | | | | AAAGATGAAAGCTTTTATGATTCCTTTAGAAAAGA | |
| | | | | | ATTGCTTTGGGACTTTATCATA | |
| 55 | 213757 | | 55 | | CTCCGCCAGACAGAGGTGCTGGGGCTGTGCAGGAA | |
| | | | | | ACGAAGTGATTAGAAATCCCGGAAAAACACACAAG | |
| | | | | | CAGGCGTTGTCATGGTGACTGGGAAAAACACACAA | |
| | | | | | GCTGGCGTTGTCATGGTAATGGAGTGTAGGACAGG | |
| | | | | | CCTGGAGCCCCTCGGTCTCTTGCTGGCGGCTGGCAC | |
| | | | | | AGAGACGGGCTGCCGTGGGCTCTGACCTTAATACC | |
| | | | | | GGGTCACAGTCGCTTCTAGGACCAAGAGGACAGAG | |
| | | | | | ACCCCATCACCGTATGCAGGGGCCTGTTCCAGGCA | |
| | | | | | GACTGCCCAGTGCCCAGCTGAGCCTCGGGTGCAGT | |
| | | | | | GCGACCCCCGCAGGGCATGTCCAGACCCCAGGACC | |
| | | | | | CCCTCTCAGGTCTAGAAGATCCAGTTGGGCAGTGTT | |
| | | | | | GGTACCACCAAGAGTAGACAGGACAGAGGATCAGA | |
| | | | | | GACAATCCCACCCAGCAGGACCCAAGGACTCAGGC | |
| | | | | | AGTGGCTTTTCAGGTGTGTGGGCCGAGGACTGGGG | |
| | | | | | AGTCGGTGAATTCTGGGGCCCCTGGGGTGGCCGTTC | |
| | | | | | AGGAACTGCAGCAGCTCCCCCCACCACAGATGCTC | |
| | | | | | GCTGCCTACTGAAGCGGCCACGTGTTTGAATGAAG | |
| | | | | | AGCAGTTAGAGGAACGCTTGCAAGAGAATGTGTTT | |
| | | | | | ATTACCTGAGGTTATGACAATACAGAACATACAAT | |
| | | | | | GTTTTCTGTGGAAAATGTGATACTACAGAGGAAAA | |
| | | | | | GGTCACTTTAATTTAAATGGCAATAGAAGTAACAG | |
| | | | | | CATTGCAAGGTGGGGTGCAGCAGCTCACGCTTATA | |
| | | | | | ATCCCAGCACTTTAGGAGGCTGAGGCGGGTGGATC | |
| | | | | | ACTTGAGGTCAGGAGTTCAAGACCAGCCTGGGCAA | |
| | | | | | CATGGTGAAACCTCGTCTCTACTAAAAATATAGAA | |
| | | | | | ATAGCCACGCGTGGTGGTGCGCGCCTGTAGTCCAA | |
| | | | | | GATACTCAGGAGGCTGAGG | |
| 56 | 404343 | | 56 | | CCCACTTTCTCAAAGTTTCTCTCTTTAGTCACTTTGT | |
| | | | | | ATTAGATTCATCCATTTTAAAAATCTTTGCTTTAGA | |
| | | | | | AGCATTGTTAATGTTTTTTGTCCATTTCACTAGAGTCC | |
| | | | | | CTGAGGAACATCATCTTGGGTTTAACAGTATTAATT | |
| | | | | | GACCACCCACTATGTAGCCAGCTATGTGCTAAATGC | |
| | | | | | TGAAAAAAATAAGAATACGTTGCAACCCTGTCATT | |
| | | | | | GAGGAGGCATATTAGTAGATTTCTGCTGTGACAAT | |
| | | | | | ATTGCATATCACACAATCCCAAAATCTCAGTGGCTT | |
| | | | | | ACAATTGCAAACATTTATTTCATGTTCATGGGTGTG | |
| | | | | | GAGGTTGGCTGTGGTTCAGCTGTGTCACTAGGCTGA | |
| | | | | | ACTTACTCAATAAGCCACATAACTTCGAGTCAGGTT | |
| | | | | | CCAGTCCATTGTATGTGTATTTTTCAAAATCTAGGC | |
| | | | | | TAAAGGAGGAACAGTCATGTGGGTCCTACTCTTCCT | |
| | | | | | ATGGTGGAAGGTTTAAGCTTAAAAGGGTTGGTGAT | |
| | | | | | TATTATGCCTTAAAGTCTTAGCTCAACAGTGGTACA | |
| | | | | | GTGCAATGTCTTCCATTTCTGTTACCAAAGCGAGTC | |
| | | | | | ACAGGACCAAGCCCAAAGTCAATGACATTAGTCAA | |
| | | | | | TGTACTCTCCTGGTAGGAGGTCTTGCAAAGGTCAT | |
| | | | | | GTTGCAAAGAGTGAGGATATATAATATTACTAGAG | |
| | | | | | GGAGGAGGTGCCTAATTGGGAAGAATAATCCAGTC | |
| | | | | | TAGGCTGCGCACAGTGGCTGAAGCTTGGAAACCCA | |
| | | | | | GTGCTTTGGGAGGCTGAAGTGGGAGGAGATCGCTT | |
| | | | | | AAGGCCAGAAGTTCGAGACCAGCCTGGGCAACCTA | |
| | | | | | GTTGAGACCCTAGCCC | |
| 56 | 404343 | | 275 | | TCCGGTGGGTTCTTGGTCTTGCTGACTCAAGAATG | |
| | | | | | AAGCTGCAGACCTTCGCAGTGAGTGTTACAGCTCTT | |
| | | | | | AAAGATGATGTGTCTGGAGTTTGTTCCTTCAGATGT | |
| | | | | | GTCTGGAGTTTGGTGGGTTCATAGTCTCGCTGACTT | |
| | | | | | TAAGAATGAAGCCACGGACGTTCGCATGTTACAGC | |
| | | | | | TCTTAAAGGTGGTGTGGACCCAAAGAGTGAGCAGC | |
| | | | | | AGCAAGATTTATTGTGAAGAACAAAAGAACAAAGC | |
| | | | | | TTCCACAGCGTGGAAGGGGACCCAAGTGGGTTGCT | |
| | | | | | GCTGCTGGCTGGGGTGGCCAGTTTTTATTCCCTGAT | |
| | | | | | TTGTCCCCACCCACGTCCTACTGATTGGTCCATCTT | |
| | | | | | ACAGGGTGCTGATTGGTCAGTTTTACAGAGTGTTGA | |
| | | | | | TTGGTGCGTTTACAAACCTTTAGCTAGACACAGAGC | |
| | | | | | GCTGACTGATGCCTTTTTACAGAGTTCTGACTGGTG | |
| | | | | | CATTTACAATCCTTTAGCTAGACGTAGAGCGCTGAT | |
| | | | | | TGGTATGTTTTTACAGAGTGCTGAGTGGTGCGTTTA | |
| | | | | | CAATCCTCCAGCTAGACACAGTGCTGACTGGTGAGT | |
| | | | | | TTTTACAGAGTGCTGATTGGTGTGTTTACAATCCTC | |
| | | | | | TAGCTAGACACAGAGCGCTGATTGGTGTGTTTTTAC | |
| | | | | | AGAGTGCTGATTGGTGCGTTTACAGTCCTCTAGCTA | |
| | | | | | GACACAGAGTGCTGATTGGTGTGTTTTTACAGAGTG | |
| | | | | | CTGATTGGTGCATTTACAATCCTTTAGCTAGACACA | |
| | | | | | CAGCGCTGATGGTGCGTTTTCTACAGAGTGCTGACT | |
| | | | | | GGTGCATTTACAATCCTCTAGCTAGACAGAAAAGTT | |
| | | | | | CTCCAAGTCCCCACTCAACCCAGGAAGTCCAGCTG | |
| | | | | | GTTTCACCTCTCACTAGCACTTTGGGAGGCTAAGGC | |
| | | | | | AGGAGGCTTACTTGAGCCCAGGAGTTTGGGACCAG | |
| | | | | | CCTGGGAGACATAGTGAGACCCTATCTCTTTAAAAT | |
| | | | | | AAAATTAGCCAGGTGTGGTGGTGGTGTGCATCTGTA | |
| | | | | | GTCCCAGCTACACTAGTGGCTGAAACAAAAGGATT | |
| | | | | | GCTTGAGCCTAGGTGGTCAAGGCTGCAGTTTTGAG | |
| | | | | | CTGTGATCATGCCATTTCACTCAAGCCTCGGTGACA | |
| | | | | | AGGCAAAACACTGTCTATATAATAATAATAATAAT | |
| | | | | | AAATAATCCATCTCACATATTCTTGTGAAAACGAAA | |
| | | | | | GGAATGTATGAATAAATGTTTTGTAAGTTGCACAGC | |
| | | | | | ATTATGAGTTTAAGTTGAGGAATTTAGGAGTGTATA | |
| | | | | | TATTTTTATATCCTGCCTGGTTCCAAAGAGGTTTAC | |
| | | | | | AGTGGCTCAGATCTAATGTGTTATTTTTCCTCCATC | |
| | | | | | ACCAGGATACTTGGTGGTTACTTAGTACAGGTTTAT | |
| | | | | | GAAATTAAATTGAATGCAAGTCTTCATGAAGAAGA | |
| | | | | | AAGATTGGGCTGAAAGTTTAGCTTTTTTGCTCTAGCT | |
| | | | | | GCTTCTGGTTTTTGAGTTATATCATTAGAAATACCA | |
| | | | | | GATAACAAGTGAAAAGTCATTCAGCTCCTTTCATTT | |
| | | | | | AAAATCTTGACAGTTTTCTTTTTTTAAGGTCAACCA | |
| | | | | | GCAAATGATATCCTGCCTCTTGAAAACTTAATCATT | |
| | | | | | TTATCTGACAGGAGTTAGATTAGGTGTCTCCAGAGC | |
| | | | | | ATTTGCTTATACTTAAAGTGCCAGAAGAGGTTCTCA | |
| | | | | | GTCCTAACAAAACAAACAAAAAAACCCACTTTCTC | |
| | | | | | AAGTTTCTCTCTTTAGTCACTTTGTATTAGATTCAT | |
| | | | | | CCATTTTAAAAATCTTTGCTTTAGAAGCATTGTTAA | |
| | | | | | TGTTTTTGTCCATTTCACTAGAGTCCCTGAGGAACA | |
| | | | | | TCATCTTGGGTTTAACAGTATTAATTGACCACCCAC | |
| | | | | | TATGTAGCCAGCTATGTGCTAAATGCTGAAAAAAA | |
| | | | | | TAAGAATACGTTGCAACCCTGTCATTGAGGAGGCA | |
| | | | | | TATTAGTTAGATTTCTGCTGTGACAATATTGCATAT | |
| | | | | | CACACAATCCCAAAATCTCAGTGGCTTACAATTGCA | |
| | | | | | AACATTTATTTCATGTTCATGGGTGTGCAGGTTGGC | |
| | | | | | TGTGGTTCAGCTGTGTCACTAGGCTGAACTTACTCA | |
| | | | | | ATAAGCCACATAACTTCGAGTCAGGTTCCAGTCCAT | |
| | | | | | TGTATGTGTTATTTTCAAAATCTAGGCTAAAGGAGG | |
| | | | | | AACAGTCATGTGGGTCCTACTCTTCCTATGGTGGAA | |
| | | | | | GGTTTAAGCTTAAAAGGGTTGGTGATTATTATGCCT | |
| | | | | | TAAAGTCTTAGCTCAACAGTGGTACAGTGCAATGTC | |
| | | | | | TTCCATTTCTGTTACCAAAGCGAGTCACAGGACCAA | |
| | | | | | GCCCAAAGTCAATTGACATAGTCAATGTACTCTTCC | |
| | | | | | TGGTAGGAGGTCTTGCAAAGGTCATGTTGCAAAGA | |
| | | | | | GTGAGGATATATAATATTACTAGAGGGAGGAGGTG | |
| | | | | | CCTTAATTGGGAAGAATAATCCAGTCTAGGCTGCGC | |
| | | | | | ACAGTGGCTGAAGCTTGGAAACCCAGTGCTTTGGG | |
| | | | | | AGGCTGAAGTGGGAGGAGATCGCTTAAGGCCAGAA | |
| | | | | | GTTCGAGACCAGCCTGGGCAACCTAGTTGAGACCC | |
| | | | | | TAGCCCAAAAAAAAAAAAAAAAAAAAAAAAAAAAA | |
| | | | | | AG | |
| 57 | 30507 | | 57 | | CAGGCATGAGCCAATATGACCAGCTCAAACATCTT | |
| | | | | | CTTTTTAAATGTCAGAAGCATGTATAGTGATTATTT | |
| | | | | | CTTATTTTTTCCCCCTTGATCCATCTCACCAGATGTT | |
| | | | | | TGTTGATTTTATAAGAATTTTCAAACTACCAGCTTC | |
| | | | | | TGGCTTTGTTGAACTTGGATTTCTGTTTCACTAATTT | |
| | | | | | TCTTTCTCCTGTCTTTGTACTTACTTTGTTGCTCTTTT | |
| | | | | | TCTAAGTTTTAAAGATGGATGCCAATCTCAGGCTTC | |
| | | | | | TTTTCGTGTGTGTATGTGCGTATGTCCATAAATTCTC | |
| | | | | | TTCTAATTACAGTGTAAGCCGCATCCCACAAGTTTT | |
| | | | | | GATAGTCACAGAACTGTATCGTCACACTATTTTTTA | |
| | | | | | ATTTCAGTAAGTTCTTCACTGATCCCTGTGTAATTTA | |
| | | | | | GAAATGTTTCATAATTTCCCTACATTGGAGGGGAAG | |
| | | | | | ATAGTTTTGTTTTTATTATTAATTTCTAGCTGTATTG | |
| | | | | | AGCTCTTGTCAGAGAATATGGTTTATTTTAGTCGTT | |
| | | | | | TGAAATTTAAGATCTGCTTAATGGCAAAATGTATGG | |
| | | | | | TCAGTTTTTGTAAATGTTGCCAGTAAGCTTGCGAAT | |
| | | | | | CATATGTACTCTAGTTTTGAAATCCATTGCTCAGTG | |
| | | | | | GATGTTCATTAGGCCAATTTGTATAATCATGTTGTA | |
| | | | | | CAAATCTATTCTATTCTTAACTGTTTTTTGTTTTAAA | |
| | | | | | GGTGTGGGGTCTTACTATGTTGCCCCGGGCTGGACTC | |
| | | | | | AAATTTCCTCAGCCTCCCAAGTATCTAGAACTACAGG | |
| | | | | | CACGTGCAGCTTGGTTTAAAAAAAAAAAAAAAAAAT | |
| | | | | | CAGTGAGAAGAGGATTTGTTGATCTCCCCGTTAGGA | |
| | | | | | TTATGGGTTTGTCTGTTCCTCCTTCTCAGCTTATGCT | |
| | | | | | GTATATATTTTGGGGCTGTGTTATTAGGTGCATCCA | |
| | | | | | AGTGTATAGTTGTTATAGTTACCATGTGAGCTCAAC | |
| | | | | | CTTGGATCTTTACATAGAGATTCTCTGTATTTAGTA | |
| | | | | | ATGTTTTGTTCTTAAAATCTGCTTCCATCTAACATTA | |
| | | | | | ATATAAATGTACCAGCTTTATTTTATATGTATGTTTC | |
| | | | | | TTGGACTTTGTCTTTATGTATTACAAGAAATTGTGA | |
| | | | | | TAAAGACCTCATTTAATGGATTGTGAAAGGACTA | |
| | | | | | GGCCATTCTGGGTCATTTACTTTTCTGAAAAATATT | |
| | | | | | TTTATTTTCTTGGTATTTAAAAAAAGGTTTATAAGA | |
| | | | | | CATTCTAATTTATCTTAGTTTTCTTCCTTCATTTATTT | |
| | | | | | AGGGGTCTGGTATCTTAGGGATATCATTCTGAAAAT | |
| | | | | | TAAACTTTTCTACATAGGACCATAGATACAGGGTGA | |
| | | | | | CTAGATGACTGGG | |
| 57 | 30507 | | 276 | | TCTGTCATCGAGGCTGGAGTGCAATGGTGCAATCTT | |
| | | | | | GGCTCACTGCAACCTCCACCTTCCAGACTCAAGTGA | |
| | | | | | TTATCGTGCCTCAGCCTTCTGAGTAGCTGGGATCAC | |
| | | | | | AGGCGTGTGCCACCATTCCCGGCTAATTTTTGTATT | |
| | | | | | TTTAGTAGAGACAGGTTTTTGCCACGTTGGCCAGTC | |
| | | | | | TGGTCTCAAGCTCCTGACCTCAAGTGATCCACATGC | |
| | | | | | CTGGTTTGACCAAATTGCTGGGATTACAGGCATGAG | |
| | | | | | CCAATATGACCAGCTCAAACATCTTCTTTTTAAATG | |
| | | | | | TCAGAAGCATGTATAGTGATTATTTCTTATTTTTCC | |
| | | | | | CCCTTGATCCATCTCACCAGATGTTTGTTGATTTTAT | |
| | | | | | AAGAATTTTCAAACTACCAGCTTCTGGCTTTGTTGA | |
| | | | | | ACTTGGATTTCTGTTTCACTAATTTTCTTTCTCCTGT | |
| | | | | | CTTTGTACTTACTTTGTTGCTCTTTTTCTAAGTTTTA | |
| | | | | | AAGATGGATGCCAATCTCAGGCTTCTTTTCGTGTGT | |
| | | | | | GTATGTGCGTATGTCCATAAATTCTCTTCTAATTAC | |
| | | | | | AGTGTAAGCCGCATCCCACAAGTTTTGATAGTCACA | |
| | | | | | GAACTGTATCGTCACACTATTTTTTAATTTCAGTAA | |
| | | | | | GTTCTTCACTGATCCCTGTGTAATTTAGAAATGTTTC | |
| | | | | | ATAATTTCCCTACATTGGAGGGGAAGATAGTTTTGT | |
| | | | | | TTTTATTATTAATTTCTAGCTGTATTGAGCTCTTGTC | |
| | | | | | AGAGAATATGGTTTATTTTAGTCGTTTGAAATTTAA | |
| | | | | | GATCTGCTTAATGGCAAAATGTATGGTCAGTTTTTG | |
| | | | | | TAAATGTTGCCAGTAAGCTTGCGAATCATATGTACT | |
| | | | | | CTAGTTTTGAAATCCATTGCTCAGTGGATGTTCATT | |
| | | | | | AGGCCAATTTGTATAATCATGTTGTACAAATCTATT | |
| | | | | | CTATTCTTAACTGTTTTTGTTTTAAAGGTGTGGGGT | |
| | | | | | CTTACTATGTTGCCCGGGCTGGACTCAAATTCCTCA | |
| | | | | | GCCTCCCAAGTATCTAGAACTACAGGCACGTGCAG | |
| | | | | | CTTGGTTTAAAAAAAAAAAAAAAAATCAGTGAGAA | |
| | | | | | GAGGATTTGTTGATCTCCCCGTTAGGATTATGGGTT | |
| | | | | | TGTCTGTTCCTCCTTCTCAGCTTATGCTGTATATATT | |
| | | | | | TTGGGGCTGTGTTATTAGGTGCATCCAAGTGTATAG | |
| | | | | | TTGTTATAGTTACCATGTGAGCTCAACCTTGGATCT | |
| | | | | | TTACATAGAGATTCTCTGTATTTAGTAATGTTTTGTT | |
| | | | | | CTTAAAATCTGCTTCCATCTAACATTAATATAAATG | |
| | | | | | TACCAGCTTTATTTTATATGTATGTTTCTTGGACTTT | |
| | | | | | GTCTTTATGTATTACAAGAAATTGTGATAAAGACCT | |
| | | | | | CATTTAACTGGATTGTGAAAGGACTAGGCCATTCTG | |
| | | | | | GGTCATTTACTTTTCTGAAAAATATTTTTATTTTCTT | |
| | | | | | GGTATTTAAAAAAAGGTTTATAAGACATTCTAATTT | |
| | | | | | ATCTTAGTTTTCTTCCTTCATTTATTTAGGGGTCTGG | |
| | | | | | TATCTTAGGGATATCATTCTGAAAATTAAACTTTTC | |
| | | | | | TACATAGGACCATAGATACAGGGTGACTAGATGAC | |
| | | | | | TGGGCT | |
| 58 | 436679 | | 58 | | GGCTTGGAGGTATGGGTAAGCAGGGAGACAAAGGG | |
| | | | | | TACAACACTTCAGATGCAAGAAATGAGTTCTGGTA | |
| | | | | | AGCCACTGCACAGCATGGTGACTACAGTTCATAAA | |
| | | | | | AACGTGAGACACAGTGGCATGCATCCATAGTCCCA | |
| | | | | | GCTGAGAGGCTAAGGGCAAGAAGATCACTTAAGCC | |
| | | | | | CAGGAGTTCAAGTCCAGCCTGAGCAACATAGGGAG | |
| | | | | | ACCCTGTGTCTACTAAATATACAAAAATTAGCTGGA | |
| | | | | | GATGGTGGCAGGCTCCTGTAGTCCCAGCTACACAG | |
| | | | | | GAGGCTGAGGCAGGAGAATCGCTTGAACCTGGGAG | |
| | | | | | GCAGAGGTTGCAGTGAGCTAAGATCGTGCCATTGC | |
| | | | | | ACTTAGTCTGGGCAACAAGAGCAAGACTCCGTCTC | |
| | | | | | AAAAAAAAAAAAAAAAAAAAAAAAGCCCACAAAAACC | |
| | | | | | AGCAAAAAATCCTCTGCCCCATCACCCCAGTTTGCCT | |
| | | | | | CACCAACAGCCTCTCCCAGACCAGGAAGCTGTTTTTT | |
| | | | | | ATTTTAACTTCATGCAAATGTTGCTAATACAAGATA | |
| | | | | | TATTCATTTTTTTAACTTACCCTTTTTTACAAAAAAG | |
| | | | | | ATGGTTCTGAAATTGAACTGTATTTAATGTCTTTAA | |
| | | | | | TGGTGAAAAAAGGAAAAGTCATAGATGACATGTCA | |
| | | | | | TTATTTTGTAAAATAATAAGATCATGGTCTGGTACT | |
| | | | | | CACTTTTGGCAGCACATATAATAAAATTGGAAAGAT | |
| | | | | | C | |
| 59 | 899656 | | 59 | | ACAACTATAATTTGACTTCGGAATAAAATTTCTTTC | |
| | | | | | ATCAGAAATGTATGTTTTGATAGGTGCACTGCATAG | |
| | | | | | GATTCTAATAGCTCTAAAATCTGCTTCAATTCAGAG | |
| | | | | | CTGTGATCTTCATCACCCCTAAGCCTATATCTTACT | |
| | | | | | CTCCACAAATTAGACTGCATCCTTAAAAGGCATCCG | |
| | | | | | CTGACAGATTTCACAGGGACTGAAGTGGGCTGGGA | |
| | | | | | ACTGCATTCCATGGCATCTGAGCTTCCCTTAGACAG | |
| | | | | | GCCAACTTCGTCATTCAGAGCAAGCACTGAATAAA | |
| | | | | | TCTCCTCCAACTTACATGAATGTAACCCACTTCATG | |
| | | | | | ACTGTCAGAGGGAAGAAATAAGCCTTTGAGAATCC | |
| | | | | | TCTGTTTCTAACAGGGCTCCCCTCATGATAATGCCTA | |
| | | | | | GACCGGTGGCCAGAGTTCCCACAGCCGAGGCTCCA | |
| | | | | | GGTACAGATGCTAAATGCTGGCCCAGAGGGTCAGC | |
| | | | | | AGGATGAGCTAGTTTCTAAGTGAAAGACTCTCATTA | |
| | | | | | CGCAAATGAGTGCTTAGGGCCTTAACACTAACCAA | |
| | | | | | TTCACACAGGTCTGACGGGGCATGAGTGTGCAAGT | |
| | | | | | GAAAGCCATGCAGGTTCCTGAGACAGCCACAGTCG | |
| | | | | | GTGGGGATCCATCAGGGGCCGGCCTCAATCCCAGC | |
| | | | | | ATTTTGGGATTTGTTACGCTTGTATGTTCTATGCATT | |
| | | | | | ATGTACAGTATTCTTACCAACAAGTAAGCTAGAGA | |
| | | | | | AAAGACATGCTATTCAGAAAATCAAAAGGAAGCGA | |
| | | | | | AAATATATTTAGCATTCATTCAGTGGAAGCGGATGA | |
| | | | | | TCGTAAAGGTCTTCATCCTCTCATCTTCATGCTGAG | |
| | | | | | TAGGTGAGGAGGAGGAGGAGGAGTTGGTCTTTGCTG | |
| | | | | | TCTCGTGGGTGGCAGAGGCAAAGAAAAGCCACGTA | |
| | | | | | TAAGTGACTCACACACTTCAAATTCGTGTTGTTCAA | |
| | | | | | GGGTCAACTGTAGTTGTTTTTAAGATGCTTCACATC | |
| | | | | | TGCTTCTAAGTCTGCTCCATCCCCATTCCCCAGCTA | |
| | | | | | ACACAACCTTTCTAAGTCAGTCCTGCATGCACTCTG | |
| | | | | | CACTCTTCCCAGGTTATTTTGTCTCTCAATGTTACAA | |
| | | | | | CCAACAGGCTCAAGCAAAGCAGGAGGATGGCTTGA | |
| | | | | | GCCCAGGAAGTGGAGGCTGCAGTGAGCCATGATGA | |
| | | | | | TCCTGCCAAAGCACTCCAGCCCGGGCAACAGAACA | |
| | | | | | AGAACCTATCTC | |
| 60 | 386674 | | 60 | | TGATTCTCTCACAGTCTGGAGGCTAAATGTCAAAAT | |
| | | | | | CAAGGTGTCAGCACAACATGCTCTCACTGAGACCG | |
| | | | | | TTAGGAGAATCCTTCCTTGCCTCTTCCTACCTTCCGA | |
| | | | | | TAGTGGCTGGAAGTCCTTGGTCTTCCTCTCCTTATA | |
| | | | | | GATAAATCACTCCAATTATTTCTTCTGTTGTCATGTA | |
| | | | | | GCCATCTGTCTTCGTGTGGTGTTCTCATATCTTATAA | |
| | | | | | GGACACCGATCATATTGGATTAAGGCCCATTCCTAT | |
| | | | | | TTCCAAGTAAGGTGACATTTAAAAGATACTGGGGGT | |
| | | | | | TAGGGCTTCAACACATGAATTTGGGAAAGGGGGTA | |
| | | | | | TGCACAATTCAACCCATAACACCAACTGTAATAAAT | |
| | | | | | TCTATATTGTTGTAAAATATCTCTTTGGTAGCAAAG | |
| | | | | | TTAGTATATGCC | |
| 61 | | Ac036181 | 61 | | GTACCTTTGTCCTTGGACTTTGGTGATGTGGTTTGA | |
| | | | | | CCCCAGCTAGAGAGTGAGGGGAACAACAGCAAAA | |
| | | | | | GGCAGGACAAAGACTGACTCGTGAGAGGAGGCCCG | |
| | | | | | GGAACAGGGGGCCATTGTGAATGAGGAGGACGTGG | |
| | | | | | GGGCCCAAGAAAGTGAGCAAAAGAGGACAGGGCT | |
| | | | | | TGCGCACTCAGTCACCAGCCCCCTTCTGGGGTCCAA | |
| | | | | | GCTGTGTCCCCCTTCTAAAGAGGTAAGCCCTGAGT | |
| | | | | | CATGGGAAGATGGAAACCGGGGCTCATGAGACAGG | |
| | | | | | ATGTTTTTTAAGCACCGTGGTGTCTTGTTGACTTGC | |
| | | | | | ACATGCACGGGGGTCTTGGGTAACCACAGGGCTCA | |
| | | | | | GGGTATTTGCAGGAACAGTTCAAGTGCTCACTTGTC | |
| | | | | | TTGGGGCTGTTTATGGGGAAGTGGTTTCCACAGTGA | |
| | | | | | GAGGAGGTGAGATATTGTTGTCACCCCGGACCACA | |
| | | | | | CTTAGCTACTTCCTTCTCACTAAAGCTCTGTAGTCAT | |
| | | | | | ATTTTCCCTGGCAGAGCAGAAACTTCTATGTTATCC | |
| | | | | | CACAGCTGTTCTAACGGTGTAGACTTGACTTATGCA | |
| | | | | | ATGATGCCAGGAGTCCTGAGCAGCACAGCCCAACT | |
| | | | | | TCAATCACACACAGATGGACAGAGCTGTATTAGCA | |
| | | | | | AAGCCTGAGCTACTGAGCGATGAGAGTACAGCCAG | |
| | | | | | GCTTTCAGACATCTGTCATTCAAGAGAGATATGCG | |
| | | | | | CTAAGCCAAGGACCTAAAGATGTGTTTAATATGGG | |
| | | | | | TGCTAATATGCATAAGGAACCTTGAAATAAATGTTC | |
| | | | | | TTAGCCTTTGGCCAAGAGGGTCCATGTCTAGGAATC | |
| | | | | | TATTCTCCATAGAAATAAATTCAAATATGGAAAAA | |
| | | | | | ATGAACAATGCATAAGTGTATTTGGTCCCCAGCATA | |
| | | | | | TTTATAGCAACTTAAAATTGGACCCAATTTAAATGC | |
| | | | | | CTATGATATGGAAATGGCTAAGAAAATTATGGGAT | |
| | | | | | CTTCCCTTGATTGGCTATTAGGCAGCCTTTACAAAC | |
| | | | | | AATGCAGTGACATGAGAAATGCTTATGTTATGGTA | |
| | | | | | AGCTTAAAAAACTCAAGATGCAAATCAGCTTATTTT | |
| | | | | | AATCAGGAGCCACCTAGCATTTGGGATGTGGTCAA | |
| | | | | | TCCCACATAATGTATTTTTGTGGGTGCAGTTCCCAG | |
| | | | | | GAAAGAGGAGGAATAAAAACGGCAAGTATGAAGT | |
| | | | | | GTCTCCTTCGCTTGCAGTCTCCTTGTCTACCCCTTTG | |
| | | | | | TCCATCCACTATGAAAGGACTCCCTTTCTGTTCCTTA | |
| | | | | | ATATGGACAATTTCTATTGAGGACTCATTGTTCTAA | |
| | | | | | GAATTGTCTCATCTCCTCCTGCATCCTCAGTGCCCG | |
| | | | | | ATCTTTTGGCTCTATGAAGGAAGGTGGGTAGTGCGT | |
| | | | | | ATGGCAGGTCCAGTTCTACCTTTCTAGTATGTTCT | |
| | | | | | GGCGTGGGTATGTAGCCCCATTTTCTAGTGGTTACC | |
| | | | | | TTGACATCATGAAGAGTTTATGTCTCTTTTGCCCTA | |
| | | | | | GGTTTTGGGCAATAGTCATTCACTGTGCAACAGGAA | |
| | | | | | ATACACGAGTCAGCATCTTATTAAAAATAAAGTCAT | |
| | | | | | TCAGGAAAGTGGACGACAGTTTCTAATCTAGAGAG | |
| | | | | | CATAGGAGAAGAAATGTTTACCACACACAAAGTAT | |
| | | | | | TAGTGCCTTTTATATCACGAAGACAAAAATAACAG | |
| | | | | | GAAAAAGACAAACACATTATAGTGAAAACTTGTTT | |
| | | | | | TTCCTAACCAGCATCTATTCTGCATGTTTCCTGATGC | |
| | | | | | CCGAAACTCACATTTCCTCAGGAAAATCTCCCTTCT | |
| | | | | | GCACCATTCTCAGGCTTTAAGTTTATGTAAAATTCA | |
| | | | | | GTAAACCCAAAGATTCAAGTTATGTGCCTTGATTAA | |
| | | | | | CTTTAAGCAAATCAATGAAACCCATCCCCATAACCA | |
| | | | | | CAGCGACAGGTTAGGAAATTTCGGTTCCTAAGTCAG | |
| | | | | | TCACATCCGAAAGGGCCTAGTGATGTTTTTTTCCAG | |
| | | | | | TGGGATCACAGACTCACTCTTCCTTGCAGAAAATGA | |
| | | | | | ACAAAGGATTCATGTAACACTGGCAGGTACTGGCA | |
| | | | | | GCCACCCAGGGCCTCTCACAGGAAAGGGAGATCAG | |
| | | | | | AAAGAGAAGCAAAGAGGACTCATGAGATACCATAG | |
| | | | | | GGCTGCTGCGTCCAGCCTTGCCTGGAGCTAGGGCCA | |
| | | | | | CCTCGATGCCCTATAGTCTTGGAGCCACAACGTGCA | |
| | | | | | TTTACTCAAAGCCTCTTTGAGTTTGGTTTGCTTGTTT | |
| | | | | | GCTTTCTGCCTGGAAACTGCCAGCATCCTGAGAGAT | |
| | | | | | ACGAGATCTGCATCTGTGCAGAGACACAGGGTTTG | |
| | | | | | TTAAAAGTCACAGGCCCTGACTGAAGTGTGGAACT | |
| | | | | | GGCTGAAATGAGAAAGTGGTAATTTGGGGAGGACC | |
| | | | | | TTGTGAAATGGAAGGAGTTTTAAACCTTACATGCAT | |
| | | | | | CAGAATTACCTGGAGCCTTGTGAAAACACAGGTTG | |
| | | | | | CTGGGCCCTAGTCCATTAAGAAAGGAAGTGGGGCT | |
| | | | | | TAGAATGTTCATTTCTCCCATGTTTCCCAGGTGATAT | |
| | | | | | TCACCATGCTGTCCTGTCTGGGCACTACCTTTTGCC | |
| | | | | | ATACCCATTACAAGGTATTGCACGTGCTGGTTGAAC | |
| | | | | | TATGGTCTGTCTTTATTTGGTGCTAAAAGCCTGTGC | |
| | | | | | CAAATACCAACGCTGCAGCATTAAGGAATGTGATA | |
| | | | | | GAAAAGATTCTGAATATAGGCCAGGCGCAGTGGCT | |
| | | | | | CACGCCTGTAATCCCAGCACTTTGGGAGGCCGAAG | |
| | | | | | CAGGCAGATCACGAGGTCAGGAGATCAAGACCATC | |
| | | | | | CTGGCTAACATGGTGAAACCCCGTCTCTACTAAAAA | |
| | | | | | TACAAAAAATTAGCCGGGCGTAGTGGTGGGCACCT | |
| | | | | | GTAGTCCCAGCTACTTGGGAGGCTGAGGCAGGAGA | |
| | | | | | ATGGCGTGAACCTGGGAGGCGGAACTTGCACTGGG | |
| | | | | | CTGAGATCGCGCTACTGCACTCCACTCCAGCCTGGG | |
| | | | | | CGACAGAGCAAGACTTCGTCTCAAAAAA | |
| 62 | | Ac040977 | 62 | | GGCCATGGGGGAAAAAGTCTAACTGGCGGAACTCC | |
| | | | | | TGGGAACTGGGGCGATGGGCTCTTAGTATCGGAGG | |
| | | | | | ATTGGAGCCATCTGATTTTTACCTGAAATTCCTTAG | |
| | | | | | TCTCTCCTGTGTTGGGGAAATGGTCACCTTGCCTTC | |
| | | | | | AGGGACCTGGGCTTTCAGCTGTCCATACCTGGCCCT | |
| | | | | | GGTTGATGGCGGCATGCTGGGCAGTGCACGTGAAG | |
| | | | | | ACGCACATGAGACAGCATCTCGTATGTTGCCCAGG | |
| | | | | | CTGGCCTTGAAAGCCTGGCCTCAAGCCATCTTCCTG | |
| | | | | | CCTCAGCCTCCCAAGTAGCTGGGATCACAGGGTTGT | |
| | | | | | GGCATCACAGCTGGCTATATTTCTTAACATATTTTG | |
| | | | | | TAACCATTCCAACCCCCAGAAATTTCTCTCTGGCTG | |
| | | | | | ACTTGATCCACAGCGCCTCCATCGCCATCCCTGAGT | |
| | | | | | GCCTTGTTGTGGAAAATCTTACTTTATCTTGGTTCTG | |
| | | | | | TTTGGTATAATCGGGGAAAGTCTGTATTCTTTCATT | |
| | | | | | ATGTAAAACAACTTATCTCTCATTGTTTCATCTCCTT | |
| | | | | | TCTGAGCTCTGCTCTGCCAGCTCTCTTTCCAAAACC | |
| | | | | | AAAATGGCTCTTCAAGTTATTTTGTAAATAATAATG | |
| | | | | | GGCCATCTACTTCTTAACATAAATGAATGATTTTCC | |
| | | | | | AAGG | |
| 63 | | Ab014087 | 63 | | CTTATTGCTGGGCAGGTTCTCATAAGAGGCCATGGG | |
| | | | | | AAAGCCATGTCCTATCTCAGGGACACAGGGTCATCT | |
| | | | | | GGGCCTCTGGCTAATAGAGGCCAAATAATGGGACT | |
| | | | | | ATTTTCCCTGTGAAATCCTGAAAACCAAAAATGGTG | |
| | | | | | GCGTCATTTCTGCATTAGCAGAGGTAATTTGCTCC | |
| | | | | | TTCTTGAAATCCAAGGTCACGTCTACTGTCTGGGGA | |
| | | | | | TTTTGATCCAGGGTCAGTGTGGTTTCTCCTTTACAG | |
| | | | | | GAGAGCCGAGTCTCAGAAAGGTGAGGTGGTTTGTG | |
| | | | | | TTGGTCATTGGCTACCTCAGATTTTAGAGCAGCTCT | |
| | | | | | ACCTTGATTGTGGGGTTGACCTAATTTTTTTTGCTGT | |
| | | | | | CTTCTTTCTTCTCCAGGTGAGGAAAGAGGACTTCCT | |
| | | | | | GTATATCTCTATCCTTTTGTTTCCATTACTCACTTTC | |
| | | | | | TGTGGCTGCTGCTGCAGAAGCCACTGCTGACTGATG | |
| | | | | | TGGATACCTCAATCTTTGGTTTACAAAAAGCCTAGG | |
| | | | | | TGTCTTTTGGCCTCTCTCCAGGTTGATAGCCATGGC | |
| | | | | | TCCTGAAAGAAATAAAAGATGATCATCTTTCTAAA | |
| | | | | | AAGTCTTAAGTCTGAATTATTAGTAACTTAACTGGA | |
| | | | | | GAATCTCACTTTTCCTACTCTCGTATTTTAACCACAG | |
| | | | | | TTGCTCTAACACAGACCTTTGAGGATCTTTTCATGA | |
| | | | | | CTTCATCACAAATACCTATTTATGCTGTACAGATG | |
| | | | | | CTACTAGGAAGGAAATAGGGATGTCTGTTTTGACTG | |
| | | | | | TGGAACTTAACTTGGTCTCGTCTCTTCGTGCATGCA | |
| | | | | | ACCCTGTCCTTGGGATAGCTTTCTTGAGCATATCTA | |
| | | | | | CTTATGTTCAAGAGGTAAATTGTCCTGAAACCCCCA | |
| | | | | | TTGCTATAAGTATTTATTTTATTACTCATAATACTTA | |
| | | | | | ATGCTCCTAAAGTTGGGGTATTTTTTTTTTGGATACC | |
| | | | | | TAAACTTCATTGAGATACTTTGAACTATTTATAGAG | |
| | | | | | AAAACGGAACCTTCTAATACCTGGCTTCTATTTCTT | |
| | | | | | AAAATGTTATGATCATACATGGCTTAGGGCTTTATG | |
| | | | | | GCCAAATAACTTCACTGAACCCAGGAAAAAGAATA | |
| | | | | | GATCCATCTGAAACAGACCTGTAGCTTCCAGAGGC | |
| | | | | | CTAAATTTTCGGCTCCATTTGTATCCTTCATTTTCTG | |
| | | | | | TGAGGTAAAGAAGTGGAAGGAGACAAGCCTCAGCC | |
| | | | | | CTTCCCCTGGCACCTTTACTCTTCGCCCTTCCTCCTG | |
| | | | | | GCATGGTGGAAAGTGCACTGGAGGAGGAGTGAAGG | |
| | | | | | GCCCTAGGTTTGCATCCATGTTCTGCCACTTGCCAA | |
| | | | | | CCTTAATGGCCCTTACAATTGATTTACCCTCATGAA | |
| | | | | | ATTTGGAATGATTTCTAAAGTCTTTCCTCGCCCTGA | |
| | | | | | ATGTTAACATTTTTTGATAGTCAGGACTTTCTGTAG | |
| | | | | | CTTCACCTTCCTTATTTAGTGTTATTTTTTTCTCAAG | |
| | | | | | ACTGAACAGAGAGGGAAGCTGTCAAAGTGTGCTGG | |
| | | | | | GCACACACCCTGCAGTGGGGCAATGGCCAATTCTA | |
| | | | | | ATCTCAAGTCATTAGGCTGCAGTAGCATGACCACTG | |
| | | | | | CTTCCTGTCTACCCTCAGAGGGTAGAGACAGCTGAG | |
| | | | | | CTCCTGTAGTTGGGGTCAGGCCCAGCCACTCTGTGG | |
| | | | | | GGACAGTGATAGTGTTGTGTCACCAATTCAGGGA | |
| | | | | | AGGAGCCACCTTGTCTTATTTTCCCTCTTGAATTATC | |
| | | | | | TTGATATGACCCCATTATAAATTTCCTTTTGTAAAC | |
| | | | | | CTCTGTCTCCCAATTTCTCCTTTTAGCTTACTTTCTA | |
| | | | | | TTGAAGTAGAGGAACAGAGTACAACTTCCATCCTCT | |
| | | | | | TTCATCAGCCCTGAAAGCAGAACGCAAGCGCCGTT | |
| | | | | | ACTGGGAACTATATCCTTGGCTCCCTGGATGTGGCT | |
| | | | | | ATTAACTTCTGGCCTGCCACTCTATCACATACACAT | |
| | | | | | ATGGAGATGGTGTCATCCATGTACCTTACCCCGTAT | |
| | | | | | TTACAACTTCTATCACCCAACAGTGCCAATGGCCCT | |
| | | | | | GATGGTCCCTCTGGGAGGGAGAGAAGAGTAAGCTG | |
| | | | | | GAGTCACCCCTTCCCTGTACTTCCCACCTCGCCAGG | |
| | | | | | CCTGTTGGTGTTAGTGTCCCTTCTGATCTTGGCCTGA | |
| | | | | | CCCCTGTGCCCTGGGCACTGGGCTGCAGGTTGGAG | |
| | | | | | AGGCAGCATGATGGAGTGGGGATAACACATACTCC | |
| | | | | | AAAACCAAACAGAAGCCAGACCTGGGTTGGGTCCT | |
| | | | | | GGCGAAACAGTCTAGAGGCTTGGTGACCTTAACCT | |
| | | | | | CCTAATTAATCTTCCTAAGCATAAGTTTCCTTATCAT | |
| | | | | | AAGTTATGTATGATAAAATTTTCCTTGGATGCATTC | |
| | | | | | ATTTTAGCATGACTTGAAATTATGTGTGAAGGAACC | |
| | | | | | TGGCCCATGGAAGTTGCCCTGTAAATTCAGATTCAC | |
| | | | | | TTTCCCTTGGACATATGGATGACATTAGCTCATTAC | |
| | | | | | AGTTATGACCTCCCTAAAACTCCCAAATATTCTTTA | |
| | | | | | AGTTCTTCTTATTTTCCCTTTAGTTTGTAGTCATA | |
| | | | | | TTTCTTAGTTCTTATATCAGTTGGGATTCCCACATCT | |
| | | | | | TCTAGTTGGACAATATTGGAGAAGACACCACATTTT | |
| | | | | | AACTGAGTTCCAGTGATATGACAGGCTTTCAATTCT | |
| | | | | | CTAATCTCACAGAAGTTAGAAAAAAAGTAGTATAAT | |
| | | | | | CAAAATCCACAGAAAATATAGAAGATTCCATTAAC | |
| | | | | | TCTGAGAATGATTCTCAGGTATCCTTAGGACCTCAA | |
| | | | | | GAAAGCTGTTCTCTCCTGGGCCTGTAGAGAGTTCAA | |
| | | | | | GTGCCAGGAATCTACCACAAAGTAGCCGGGAGGTG | |
| | | | | | CAGGGCAGCAGGGGGCACAGTGAAGTGCTGAAGG | |
| | | | | | GCTTCTCAGTCTTCTTTAATTAGAGTGAGAAGAAAA | |
| | | | | | GAGCACCTCCTCATTTTAGAGTACAAGGTGTGAACT | |
| | | | | | CACTCTCAGCTGCCAAGTGAGCTTCACCTTGGGCTG | |
| | | | | | TTTTGCATGCTTTCTCCTAGTGCTTTAAGCCACCCTG | |
| | | | | | AGATGTACAGACCAATACTGGCCATCACAAAAATA | |
| | | | | | TACTCGAGTACATAGACCATTGACACTATAAAGCA | |
| | | | | | AGTAAACAATGAAGTCTACATAACAGCCAAATAAC | |
| | | | | | AACATGATGATAGGATCAAATCTGCACATATCAAT | |
| | | | | | ATTAACCTTGAATGTAAATGAGCTAAATGCCTCAAT | |
| | | | | | TAATAGGCAGAGAGTGGCAAGTTGGACAGAGAAGC | |
| | | | | | AAGACCCAACTGTATGTCTTCAAGAGACCCATCTCA | |
| | | | | | TATGCAGGGACACCAATAGCCTCAAAGTAAGGGAT | |
| | | | | | GGAGAAAGATCTATCAAGCAAATGGAAAACAAAA | |
| | | | | | AACAGCACTCTCTGTCCAACAAAAACAGAATATAC | |
| | | | | | ATTCTTTTCAGCTGCACATGGTACATACTCTTAAAA | |
| | | | | | TCGACCACAATTGCTTTATTGGCCAGAAAGCAATTC | |
| | | | | | TCAACAAATTCAAGAAACCTGAAATACCGGCCAGG | |
| | | | | | TGTAGTGGCTCACACCTGTAATCCCAACACTTTGGA | |
| | | | | | AGGCTGAGGTGGGCAAATCACTTGAGGTCAAGAGT | |
| | | | | | TTGAGACCAGCCTGGCCAACATGGCAAAAACCCAT | |
| | | | | | CTCTTCTAAAAAATATAAAAATTAGCCGTGCATGGT | |
| | | | | | GGCATGCGCCTGTAGTCCCAGCTACTTCGGAGGTTG | |
| | | | | | AGTCACGAGAATTGCTTGAACCTGGGAGGAGGAGG | |
| | | | | | TTGCAGTGAGCTGAGATCACGCCATTGCACTCCAGT | |
| | | | | | CTGGTTGACAGAGTGAGACTCATCTCAAAAAAACA | |
| | | | | | AAAAAACCCTGAAATACCAACCACACTCTTGGACC | |
| | | | | | ACAGTGCCATAAAAATAAATACCAAGAAGATCTCT | |
| | | | | | CAAAACCATATAATTAAGTGGAAATTAATCTACTCC | |
| | | | | | TGAATGACTTGGGTAAACAAAGAGAAATTAAGGCA | |
| | | | | | GAAATCAAGAAATTGTTTACAACT | |
| 64 | | A1136332 | 64 | | CCGTGGGGCTACTTCCAGTTCAATGTGACCAGCAGA | |
| | | | | | AGGCACAGTACTTTACAGGTCCTGCAGAATGGAGG | |
| | | | | | GCGTGTAACCAGCCTGGAGCAAGGAAAGAGGGCGT | |
| | | | | | CCTGCAGACAGGGGTGCCTGCGCTAGGTTTTGAAG | |
| | | | | | GATAACAGGTTGGCCAGAGCAGACAGGAACAGAA | |
| | | | | | GAACCCCTTCTAGACTATTTGAAGACAATTCTCCAT | |
| | | | | | GGGTCGCTTGCATTTCTGCATGTATAGTGAAAAGTC | |
| | | | | | TTTGACAGCTTTTATTCCAGACTGTCTTTTTAAGAGT | |
| | | | | | ACTTGAGTATCTCAGATGATCCAGATAGTTTTCTCCC | |
| | | | | | TCCTGGAAAGAGAGCAGATTTTTCCTCCTGACCAGG | |
| | | | | | ATAATAAAATCATACCTCTC | |
| 65 | | Ac010532 | 65 | | GGCCGGGCATGGTGGCTCACGCCTGTAATCCCAGC | |
| | | | | | ACTTTGGGAGGCCGAGGTGGGTGGATCACTTGAGG | |
| | | | | | TCAGGAGTTCGAGACCAGCCTGGCCAACATGGTGA | |
| | | | | | AACCCCATCTCTACTAAAAATACAAAAATTAGCCA | |
| | | | | | GGCGTGAGCCACTGCGCCCGGCCAGAATGGCATTA | |
| | | | | | TATTTAAATAGTTCATAAAGAAGCACAAAAGAATA | |
| | | | | | TATTTCATAACATGTAAAAATTATATAAAACGTAA | |
| | | | | | ATTTCCATGTTGATAAATAAAGTTGTATTGGAACAC | |
| | | | | | G | |
| 66 | | Ac010611 | 66 | | CAAGAATATAGACCTTACACATAAATAGTTCAGAA | |
| | | | | | AGGTTGAACAACTAAAAGATAGGGACTTTGATAAG | |
| | | | | | TTATGACATATTTTTTGGAATCAAGGAGATTATGT | |
| | | | | | ACATGCATAAAGCTGTGTGCATACTCAGGAAAAAG | |
| | | | | | CTGAGAAGGCCCTAAACTCTCACCAATGGCTGACCT | |
| | | | | | TGAGGCACTGCATAAGTAGGTGAAGGCTAAGGAGA | |
| | | | | | AGCTGTTAACTTGTGGCTAAGTATTAAAGGTGTGCC | |
| | | | | | CCAACACACAGAGTCCCCAATACAAAGAGAAGTAT | |
| | | | | | TGATTCCAGGCATTTAAGGAAATCTGTCCAATTATT | |
| | | | | | AGCACACTACTAAGCATATGAATCAGATATTTCATA | |
| | | | | | CACAACAAAGAATATAGACTTTACAAATATATAGT | |
| | | | | | TCAGAAAGGTCAGTAAACAGCAAAATATAGCAACA | |
| | | | | | ACAGCAAAACCTGGTGAGGAAAGGGAGTCTGATAT | |
| | | | | | ACAGAGTTGTAACATGTTATTAAAATGTCCAATTT | |
| | | | | | TCACCAAAAAATTATGAGACATGCACAAAAACAAG | |
| | | | | | CAAGAATGGTCCATGCACTGATGGGGAAAAAAGCA | |
| | | | | | ATAGAAATTCCCTGAGGAAGCCCAGACTTCACACTT | |
| | | | | | ACTCAAAAAAGACATTGAAAACGCTATTTTAAATA | |
| | | | | | TGTTCAAAGAACCAAAGTAAACAACGTCTCACCAA | |
| | | | | | ATAGAGAAAATCAATAATGAGATAGAAATTACGAA | |
| | | | | | GAAAAGCCAAAAAGGAATGAACAGATTCTCAGAGA | |
| | | | | | CCTGTGGGACACTGTCAGATGTACCAACATAGGCA | |
| | | | | | TGATGAAAGTCTCATGTCAACCATAATTTTCACCCA | |
| | | | | | TAGCCATACATGCCCAAGAGAATTGAAAACATGTA | |
| | | | | | ATACTTGAATGTGAATGTTCATAGTGGCATAATAGC | |
| | | | | | TAAAAAAAAAGAACCCAGATATCCATCATCTGATG | |
| | | | | | ATGAGTGAACAGTGTGGTTTATGCATACAGTGGACT | |
| | | | | | GGATTCAGGCATAAAAAGGAATGAAGTATTGATAC | |
| | | | | | AGACTACAACGTGAATGGATGAGCCTTAAGAATAT | |
| | | | | | CATGCTAACAAAGAAGCTAAACACACAATATGGTT | |
| | | | | | CCACTTACATGCAATGTCCAAAATAAGTAAATCCAT | |
| | | | | | AGAGACTGAAAATACATCAGTGATTGCTAGGAGCT | |
| | | | | | GGGAGAGGGAAGAATAGTGAGTGCATGCTAATGAG | |
| | | | | | TCTGACATTTACTTTTAGAAAGATGAATGTATTCTG | |
| | | | | | GAATTGGATAGCGCTGATTATACGACCTTGTGAATA | |
| | | | | | TACAGGATCCACTGAACTGCACTTTAAAAGGGTGA | |
| | | | | | ATATTGTGTGAATCATATCTCAATTTAAAAAGATAT | |
| | | | | | ATATAAAGTTCCCTGGGTGAATACTGGTTTCCCTCC | |
| | | | | | TCCCTTCAGTATATGTGAAATGTAGTGAAATTTATA | |
| | | | | | TGGTTCTGACAGTATTTTATTTTAATGATTTTTCCTC | |
| | | | | | CATCCTTGGTAGTTTTTTTTTTTTCCTTTATGTATAT | |
| | | | | | GAAACGGCAACAGTGTTCGTGAAGTCAGAGCTACA | |
| | | | | | CAAAATACTATAATCGGAGGAGTGGCAACTCTCCC | |
| | | | | | CTTTCCCATTGTTGTCTTTCCACCCCATTCCCGCCC | |
| | | | | | GCCCCCTGTAAA | |
| 67 | | Ac0164616 | 67 | | AGCCTCCCGAGTAGTCGGGATTACAGGCGCCCACC | |
| | | | | | ACTAGGCCCAGCCAATTTTTGTATTTTTACTAGATA | |
| | | | | | CGGGGTTTCACGATGTTGGCCAGGCTGGTCTCAAAC | |
| | | | | | TCCTGACCTTGTGATTCACCTGTCTCGGCCTCCCGC | |
| | | | | | CTCAGTCCCCCAAGTAGCTGGGACTACAAGCGCGG | |
| | | | | | GTCACCACACCCAGCTAATTTTTGTATTTTTAGTAG | |
| | | | | | AGATGGGGTTTCACAATGTTGACCAGGCTGGTCTCA | |
| | | | | | AACTCCTGACCTCAGGCAGTCCTCCTGCCTGGCCTC | |
| | | | | | CCAAAGTCCTGGGGTTTACAGGCATGAGCCATTGGG | |
| | | | | | CCTGGCCTACCCTGATTCTTAAGAAAGCATTTTCTT | |
| | | | | | TCTTTCATATTATAAAGTAGTTATGTGTAGGTTTATT | |
| | | | | | TAGTTAGGAATTCCAGCTGTTCAGAGATGGCAAAA | |
| | | | | | C | |
| 68 | | Ac012357 | 68 | | ACAGTTCATCATATTGCTTCATATTTCTAGATTCCTA | |
| | | | | | GGAAATGTATTCTAGATTCATTTCTGGGAGCTAAGC | |
| | | | | | AGGAACTGTGTATACCAGTTGAATTCAGCCCATGCT | |
| | | | | | GATTGTGCACCTGTGGTTAAATAAGGTGCAGCAGG | |
| | | | | | CAAGAGAGAGAAGTATGTTTAGACAGCATCTGCCC | |
| | | | | | TCAAGGAGTTTGTAACCTAGTTGAGAATCTTGAAAT | |
| | | | | | CTGTTTTCTAGTTTGCTAGTTTCTAGTTGGCTTTAAC | |
| | | | | | TAATTAATTAATTTTAGATTCAGGATACTACTGTGT | |
| | | | | | AAGTAAAACTTAATTACATTTGACATGATACAGTTG | |
| | | | | | GCCCTCCATATCTGCGGTTTCCACATCTGTGGATTC | |
| | | | | | AACCAAACTTGGATTGAAAATATTCAGCAAAAGGC | |
| | | | | | CAGGCACTGTGGCTCATGCCTGTAATCCCAGCACTT | |
| | | | | | TGGGAGGCTGAGGCAGGCGAATCACGAGGTCAGGA | |
| | | | | | GATCAAGACCATCCTGGCTAATACGGTGAAACTCC | |
| | | | | | GTCTCTACTAAAAATACAAAAAATTAGCCGGGCGT | |
| | | | | | GGTGGTGAGCACCTATAGTCTCAGCTACTCGGGAG | |
| | | | | | GCTGAGGCAGGAGAATGGCGTGAACCTGGGAGGCA | |
| | | | | | GAGCTTGCAGTGAACCGAGATCACGCCACTGCACT | |
| | | | | | CCATCCAGCCTAGGCAACAGAGTGAGACTCTGTCTC | |
| 69 | | Ac016008 | 69 | | CGGGTTTGAACTGCGAGAGTCCACTTATATGAGGGT | |
| | | | | | TTTTGAAAATAAAAGTTACCCCGAGTGTGCCTGCCT | |
| | | | | | CTCCTGCCTTCCCTTCCACGTCCTCCACCTCTTCTGC | |
| | | | | | CTCTGCCACCCCTGAGACAGCAAGACCAACCCCCG | |
| | | | | | GCTTCTCCTCCTCAGTCTACTCAACCTGACGATCAC | |
| | | | | | AAGGATGAAGACCTTTATGACTCACCTTTATGATTC | |
| | | | | | ACTTCCAACATATGACTTTGTACAGAAATCAGGAA | |
| | | | | | GATGCTTTGAAAGAAACACTGTGCAATGAAAGTGC | |
| | | | | | CACTGATGTGTCTAGCATTGACATGCTTTTGGCTGC | |
| | | | | | AAAGACTTGAGCCCACACGTTGCCTGAAACCTTCA | |
| | | | | | GTCCTTTGAAGCTGTGATTTCAAGACCCAGATGTTG | |
| | | | | | ACAGCTGCTCAGAATGCTTCTCAGGAAGAGGCTGG | |
| | | | | | GACTTCCAAGACCCCATTCCTGGGTTGGGTGATGAG | |
| | | | | | TGGTTCTGATACTGTGAAAACTCACAAAAGACTATG | |
| | | | | | TAATGATACCAACCACGTGAGACTATTTTGAGAATT | |
| | | | | | AAATGAGTTAATATATGC | |
| 70 | 242250 | | 70 | | GCGGCCGCAAGGGCTTGGCTGGGCCGCGGGAGGCG | |
| | | | | | GGAGGTTCTTCGTCCTCCCGAGCCATCTCCCTGAAC | |
| | | | | | TGACAAGCAGGACTCCCGGGTCCAGGGGGCACAGG | |
| | | | | | GCCCGGGGCGGTGACCCTGCGGATCGGGCTGCCGG | |
| | | | | | AGGAGCCCACTGTAAATGCCGCAACTGGCCCCAAA | |
| | | | | | CACTGCGTTCCTGGACTGCACCAGCAGCTCCTGGCG | |
| | | | | | CGGCCGCAGAGTTGGTGGATATTTTCCAAGGGGGA | |
| | | | | | AAAAAATCTTTTAAATGCCATCTGTTTACTTTAAAA | |
| | | | | | ATGTTGATTACTTAAGAAAAACGAATGGATGTCTG | |
| | | | | | GGCAAAGGTATGGACGTCACAATTATTTTGAAGGC | |
| | | | | | GTCCTTTTTAACTTTAAACAGACCACGCCAGGAGGA | |
| | | | | | GACTGCTGACCCAGAGCGCATTACCTAAAATCTGGT | |
| | | | | | ACCCAGAGTGCACCCTTCGCCCTCGTTGGAGTTCTC | |
| | | | | | TCCTCTCTGCCAAGCTTTGCTCCGTGCCAGAGGTGT | |
| | | | | | GCTCCATTGTACCTCCGCTCTGTCCCTGCAGTCAGG | |
| | | | | | CAACCAATTGGAGAAGAGTATAAATAGTAATTAAC | |
| | | | | | CAGGGAGAGTTGTAATTCAGAAACCTAGTTAAAAC | |
| | | | | | AAGTCCTCAAAAACTAGAGAATATGAGAGTGGGGA | |
| | | | | | GACATTTTGAAGGCATTAAGAACAAAAAACGATGG | |
| | | | | | GGACGAATGGTTGAGTCTGAGGATCAGCATCGTAA | |
| | | | | | TCTGTTAGAGAACGAGGTCGTGGCTGTGTCTGTGAG | |
| | | | | | TCGTTAATGGGTTTAATCGGTTGATACACAGCCTGC | |
| | | | | | TAGTGGCCTAACCAGTAACCCAGGGCCTGGCAGAT | |
| | | | | | TTGCATGACATCTCGGAGTTTGATTGCTCTTCCTTCC | |
| | | | | | ACTTGGCAAAAGGAGACACCATCAGCCGGATCAGG | |
| | | | | | AGGGGTCATGGTGAGATGGAACCCACCGAGGTGGT | |
| | | | | | GTACAGAGCTGGCGCTGCCAATGGCCAGAGTGGCA | |
| | | | | | GCCTTTCTACCTCCTTAACCCTGCAAAAATCAAACG | |
| | | | | | TGCTAGTACGCACTGTCCATCCACACTGGAACTCCA | |
| | | | | | GTTGGTTTTAGTCTGCGATGATGACTCTTCTGGGTT | |
| | | | | | GACTTTTCCAGTTCACAGCCTTTCTACCTCCTTAACC | |
| | | | | | CTGCAAAAATCAAACGTGCTAGTACGCACTGTCCAT | |
| | | | | | CCACACTGGAACTCCAGTTGGTTTTAGTCTGCGATG | |
| | | | | | ATGACTCTTCTGGGTTGACTTTTCCAGTTCATATGC | |
| | | | | | AGCCCTCTTGAAGCAGGCCTCCCAAACTTAGCAGA | |
| | | | | | CACCAATGAGAACCTCACAAAGAGGCTCATCAAGC | |
| | | | | | AGGCTGGTGAAACTGGGTGTTACTTCCTGTTCCATG | |
| | | | | | GGTACCCCATAGTGTTTGGGAAACACCGGGCTGTG | |
| | | | | | GTTCAGGAGAATTTCACATATGCTAAGATGGAGAA | |
| | | | | | AGAACCTGCCCTTTACATTAGGCTTGGGATGTTAA | |
| | | | | | TTTAAAGTTTGAATGACCAAAAQATTAAATCTGTAAC | |
| | | | | | TTTTAAAGTTTCTCTTTGTGATTTTACTTAAGTGTTG | |
| | | | | | GTAGATATTCTTAAATTGTAATGACCTCAGTTTGGG | |
| | | | | | AATTAAGTTAGCCAAATATTGTGTAATTATTGTTTG | |
| | | | | | TTATACAAAAATATGCCTTAGACTGTACAGCGGCA | |
| | | | | | GAAACTCCCTCTACCACCTCGGTCCCCCTTTCCATT | |
| | | | | | CTGCGTTATACAAAATAAGCTGACACGTTAATGCTG | |
| | | | | | TGGCCCACAAAACAAAGTATACCGT | |
| 70 | 242250 | | 277 | | GGAATAATGCAGGTTCTGGGCAGGGATGGAAAGAG | |
| | | | | | TGAATGCGCTGGTACGGTAAGGTGCCTCGCAGGCA | |
| | | | | | CGTGAGGGCCTCTCTAATCGTTAGCTATTGTCACCG | |
| | | | | | ATTGTATTGTTATGACTTCTACCACCACCACTCCCC | |
| | | | | | CTCCTCCTGGGATGGTGATTCCAGGGCCAGGCGGC | |
| | | | | | AGGCTATAACTAGCGCCCTTCCAGGTGGAACCCGC | |
| | | | | | CAGAGCCCCGAGGCAGCCTAGGATTTTCTGAGATC | |
| | | | | | AGACACACTTGGGCCGGGTTGGGAGGAACTGGCAG | |
| | | | | | GAAAAGGACTGAACCTTAATGTCAGGCGGTTTTG | |
| | | | | | AAGCACCTGGGGCAAGCTATGGAAATCCCCACAGG | |
| | | | | | AAGGCTCACGCAGTCTCTTAGGCGGCTGCCCTCCAC | |
| | | | | | CTGCCACGTTCTTTTTGATTGACTAAAAAACGCTGA | |
| | | | | | ATGAAGAACGAAGTCGCGTGGAAACCCTCGCCGCG | |
| | | | | | CGCCTGCAGCGGACAGCGCAGCCCGGGAGGTTCGG | |
| | | | | | CTGCCGACTTGCGCCCGGGGGCTGCGCTGCGAGCG | |
| | | | | | GCCACGCATGGCGGCTGGACCCGGGCGGCCGCAAG | |
| | | | | | GGCTTGGCTGGGCCGCGGGAGGCGGGAGGTTCTTC | |
| | | | | | GTCCTCCCGAGCCATCTCCCTGAACTGACAAGCAGG | |
| | | | | | ACTCCCGGGTCCAGGGGGCACAGGGCCCGGGGCGG | |
| | | | | | TGACCCGGCGGATCGGGCTGCCGGAGGAGCCCACT | |
| | | | | | GTAAATGCCGCAACTGGCCCCAAACACTGCGTTCCT | |
| | | | | | GGACTGCACCAGCAGCTCCTGGCGCGGCCGCAGAG | |
| | | | | | TTGGTGGATATTTTCCAAGGGGGAAAAAAATCTTTT | |
| | | | | | AAATGCCATCTGTTTACTTTAAAAATGTTGATTACT | |
| | | | | | TAAGAAAAACGAATGGATGTCTGGGCAAAGGTATG | |
| | | | | | GACGTCACAATTATTTTGAAGGCGTCCTTTTTAACT | |
| | | | | | TTAAACAGACCACGCCAGGAGGAGACTGCTGACCC | |
| | | | | | AGAGCGCATTACCTAAAAATCTGGTACCCAGAGTGC | |
| | | | | | ACCCTTCGCCCTCGTTGGAGTTCTCTCCTCTCTGCCA | |
| | | | | | AGCTTTGCTCCGTGCCAGAGGTGTGCTCCAGTAC | |
| | | | | | CTCCGCTCTGTCCCTGCAGTCAGGCAACCAATTGGA | |
| | | | | | GAAGAGTATAAATAGTAATTAACCAGGGAGAGTTG | |
| | | | | | TAATTCAGAAACCTAGTTAAAACAAGTCCTCAAAA | |
| | | | | | ACTAGAGAATATGAGAGTGGGGAGACATTTTGAAG | |
| | | | | | GCATTAAGAACAAAAAACGATGGGGACGAATGGTT | |
| | | | | | GAGTCTGAGGATCAGCATCGTAATCTGTTAGAGAA | |
| | | | | | CGAGGTCGTGGCTGTGTCTGTGAGTCGTTAATGGGT | |
| | | | | | TTAATCGGTGATTACACAGCCTGCTAGTGGCCTAAC | |
| | | | | | CAGTAACCCAGGGCCTGGCAGATTTGCATGACATCT | |
| | | | | | CGGAGTTTGATTGCTCTTCCTTCCACTTGGCAAAAG | |
| | | | | | GAGACACCATCAGCCGGATCAGGAGGGGTCATGGT | |
| | | | | | GAGATGGAACCCACCGAGGTGGTGTACAGAGCTGG | |
| | | | | | CGCTGCCAATGGCCAGAGTGGCAGCCTTTCTACCTC | |
| | | | | | CTTAACCCTGCAAAAATCAAACGTGCTAGTACGCA | |
| | | | | | CTGTCCATCCACACTGGAACTCCAGTTGGTTTTAGT | |
| | | | | | CTGCGATGATGACTCTTCTGGGTTGACTTTTCCAGT | |
| | | | | | TCATCATGCCTTTCTACCTCCTTAACCCTGCAAAAA | |
| | | | | | TCAAACGTGCTAGTACGCACTGTCCATCCACACTGG | |
| | | | | | AACTCCAGTTTGGTTTTAGTCTGCGATGATGACTCTT | |
| | | | | | CTGGGTTGACTTTTCCAGTTCATTATGCAGCCCTCTT | |
| | | | | | GAAGCAGGCCTCCCAAACTTAGCAGACACCAATGA | |
| | | | | | GAACCTCACAAAGAGGCTCATCAAGCAGGCTGGTG | |
| | | | | | AAACTGGGTGTTACTTCCTGTTCCATGGGTACCCCA | |
| | | | | | TAGTGTTTGGGAAACACCGGGCTGTGGTTCAGGAG | |
| | | | | | AATTTCACATATGCTAAGATGGAGAAAGAACCTGC | |
| | | | | | CCTTTACATTTAGGCTTGGGATGTTAATTTAAAGTT | |
| | | | | | TGAATGACCAAAAATTAAATCTGTAACTTTTAAAGT | |
| | | | | | TTCTCTTTGTGATTTTACTAAGTGTTGGTAGATATT | |
| | | | | | CTTAAATTGTAATGACCTCAGTTTGGGAATTAAGTT | |
| | | | | | AGCCAAATATTGTGTAATTATTGTTTGTTATACAAA | |
| | | | | | AATATGCCTTAGACTGTACAGCGGCAGAAACTCCCT | |
| | | | | | CTACCACCTCGGTCCCCCTTTCCATTCTGCGTTATAC | |
| | | | | | AAAATAAGCTGACACGTTAATGCTGTGGCCCACATT | |
| | | | | | AAACAAAGTATACCGTACGTGTGTGTGTGTGTATGT | |
| | | | | | GGCATAATAAATGGTGGTAGCTAACACTTACCGAA | |
| | | | | | TGTTTTCCCTATGTTCCAGGCACTGTTTCAAGTTTTA | |
| | | | | | CAGGATTAGCAAATTTAATCCTCATTACAGTTCTGT | |
| | | | | | GAAGTAGGTACTTTTACAGGTGAGGAAACACAGGC | |
| | | | | | ACAGAGAGGTTAAGCAATTTGCCCAAGATCTCACA | |
| | | | | | GCTGGGAAGTACCAAAGCTAATATACCAACCCAGG | |
| | | | | | CAGTCCTGCTCCAGAGATCGTTCTGGACCATTCTGG | |
| | | | | | ATCACACTTCCTCGCTTAAGTGATTGAAGCAAGATA | |
| | | | | | TTTATCATATAGCATGGGTCCAAAACTGAGTTTGCT | |
| | | | | | TTAGAAGAGTTTGACAGCTTTCTGACATGCCTTTAG | |
| | | | | | TGGTCTCAGCGCAGACTGCAGATTTTGTCATTCACT | |
| | | | | | TGAAAAGAATATC | |
| 71 | 331938 | | 71 | | CACTGCGCCCAGCAGGAATATTCCTAAATATAAGA | |
| | | | | | GGTGTGTCTGCCACCCGCCCTTCTCAAGTGGAGCTC | |
| | | | | | TGGGTTGAGAGAGGGAGGGGGTGAATTTTGGGCTA | |
| | | | | | AGGAGCCTGCTGATGTCACTTTTCTTGTCTTTTCAAT | |
| | | | | | TATCTGTATTGGCTTTTTGATTGTCAAAGTAAAAAA | |
| | | | | | ATGTGAAGATTACAGGAATCATGTCCTGATAATAG | |
| | | | | | CTACCTCATATCAAGCCCTCACTATGTGCCAGGCAC | |
| | | | | | CTTCTGGGGACTTGGCTGCAGTTGTCTGTTACTCTTC | |
| | | | | | ACACAAGCTCAATGAGGCGGTCCTGTTATTACCATT | |
| | | | | | TTTATTTTAAGAATGAGGAGAATGCAGCTTCAAGA | |
| | | | | | AGGTAAGCAACTTGCCGACCGTCACACAGCTTAGC | |
| | | | | | CGAGGAAGAGCCAGGCTTCACACACGGGCCTTGCC | |
| | | | | | GCCTCTAGACTACGTGTTTATTTTTTTAGACTGAGCA | |
| | | | | | CTTTTAAAAGAGTGGCTTATTTTTTTTGTTTTGAATT | |
| | | | | | TAAAGGTCACAAAGACACACAGAAATTGTTTGCTA | |
| | | | | | TCTCTCCCAAGATAACCTCTGTGATATG | |
| 72 | 215056 | | 72 | | GTTCCCAGGCTGGGGCGATTTGCCGTCACCCCTGAAC | |
| | | | | | TTCCCCGTTCCTCTTCTCGGCTGCCTCCTTTTCCGTT | |
| | | | | | GTCCCTTCGCGCCCCAAACCACATCCTGGAGCGCAC | |
| | | | | | TCTCCAGCGTGGCTGGCAGCGGGGACGGTGCGCCG | |
| | | | | | GGGCGCAGGCCCAAGAGTCGCGTGCGCGGCCCCTT | |
| | | | | | GCACCATCCCCCCGGGCCCACCCCCGGGCCGCGCT | |
| | | | | | GATTGGGCAGGTAGGGACTCTGCCCAGCGGAAAGT | |
| | | | | | TTTGGGTGCCGGGAGGAAGTCTAACCTTTGGGAGA | |
| | | | | | CTCCAAGACAGCAGCTCCGAGGTCGGCGGGGGTCT | |
| | | | | | GGGTGGCCATGGAGGAGCCCCCTGTGCGAGAAGAG | |
| | | | | | GAAGAGGAGGAGGGAGAGGAGGACGAGGAGAGGG | |
| | | | | | ACGAGGTTGGGCCCGAGGGGGCGCTGGGCAAGAGC | |
| | | | | | CCCTTCCAGCTGACCGCCGAGGACGTGTATGACATC | |
| | | | | | TCCTACCTGTTGGGCCGCGAGCTTATGGCCCTGGGC | |
| | | | | | AGCGACCCCCGGGTGACGCAGCTGCAGTTCAAAGT | |
| | | | | | CGTCCGCGTCCTGGAGATGCTGGAGGCGCTGGTGA | |
| | | | | | ATGAGGGCAGCCTGGCGCTGGAGGAGCTGAAGATG | |
| | | | | | GAGAGGGACCACCTCAGGAAGGAGGTGGAGGGGC | |
| | | | | | TGCGGAGACAGAGCCCTCCGGCCAGCGGGGAGGTG | |
| | | | | | AACCTGGGCCCAAACAAAATGGTGGTTGACCTGAC | |
| | | | | | AGATCCCAACCGACCCCGCTTCACTCTGCAGGAGCT | |
| | | | | | AAGGGATGTGCTGCAGGAACGCAACAAACTCAAGT | |
| | | | | | CGCAGCTCCTGGTGGTGCAGGAAGAGCTGCAGTGC | |
| | | | | | TACAAGAGTGGCCTGATTCCACCAAGAGAAGGCCC | |
| | | | | | AGGAGGAAGAAGAGAAAAAGATGCTGTGGTTACTA | |
| | | | | | GTGCCAAAAATGCTGGCAGGAACAAGGAGGAGAA | |
| | | | | | GACAATCATAAAAAAGCTGTTCTTTTTTCGATCGGG | |
| | | | | | GAAACAGACCTAGATCCAAGGCCACAAGTAAGGCT | |
| | | | | | ATGGCTCTGATTCTAGAAGACAACCTTCCAAGATGC | |
| | | | | | CTGGCAAAACCACCTCCCTGTGCCACACAGACACA | |
| | | | | | CTAGGCCTGTGTATTTATTTCCCCTTCAAAGCAGAC | |
| | | | | | TGAGGAGGGAGGAGACGAGGTTCTCTTGGCATCAC | |
| | | | | | TTTCTCCCTGGCTGCAGAACTAGACACCCTTGAAGA | |
| | | | | | TTTGGCCTGGGCCAGTGAGACTGAAATCAAGAAAA | |
| | | | | | ACAGAAGGGATGTGCAGGGTGGGGGGGTCCACTTC | |
| | | | | | CTGCTCCCATGTCAACCCCCAGGGCCTCCAGCGTGC | |
| | | | | | AGACGCGTGTCCTACTCATCTGCTCCCACGGATGAC | |
| | | | | | CCTGGTCTTCAATGGTTAGCAGAAGGGAGAAAAGA | |
| | | | | | AAGCAGGAAAATGTGCTATTGAGATTCCAGTGGTG | |
| | | | | | ACTTCACTGATATTTAGTGAATATTTGATTTAGCCA | |
| | | | | | ACATGCCTTTCTTTATGTGATTTTGTATTAAAGTAA | |
| | | | | | AATGATTTTTATACTTTTC | |
| 73 | 14359 | | 73 | | GATCAAGTTCTAGAGTGGAACTATCACAGGGGCTG | |
| | | | | | TGAGGACTTGGGAGAAGAGATCATATGGTCACTTG | |
| | | | | | TTTTTTGGAAGAGATGAAGAAAGGCATGAAATAGC | |
| | | | | | CTGTTAAAAGTGAAAAGGTTAACGAAGTTTCTCAG | |
| | | | | | GGCAAAGATGAGAATCCAGCTCTGTTTCAATAGTGT | |
| | | | | | TTAGTTGAGGCAACCAGGAGATATACTAACAGTGA | |
| | | | | | TCCTGCCTCAAGGAAAAGATAAACACTTCTGGGAG | |
| | | | | | TCCATTTTATAACCCAGTCTGCCCCTGATACCATAG | |
| | | | | | AAAACTAGTAAAAGCAGCTGTGGGTCCCCAAACTT | |
| | | | | | CTATGGAACAGCTTTGGATATGGCATTTTTAGTTTT | |
| | | | | | TAATAACAGGGAAAAAGTAGAGGAAGCAAAAAGA | |
| | | | | | GCAAGAAGGACCTCCCACAAGGTGCAGCTCTTGGT | |
| | | | | | TGCAACCTTAAGCTAACCTCCCACATGGGGCTGCCT | |
| | | | | | CCTGAGTCTTGGCCTGAACAATAGAAACTGAAAGG | |
| | | | | | TGGGAAGACCAAAGCTGGCCATCTGAGTCACTGTG | |
| | | | | | CCTTGGGCATAAATCAGGGTGCACACTGTAAGAAA | |
| | | | | | ACTGGCCATTGGAAGAGGGATAATCCAGTGTTCTG | |
| | | | | | AAGAGAGCCATCGGCACCCTAACTAATGATGAGTT | |
| | | | | | AAACAGCCAGGCAAGTGCCCAAAAGTGATGGGGCC | |
| | | | | | CGAGACCTTCCACCAAAGCTCCAATCAGACAACTA | |
| | | | | | GCCATATTATCTGGAGAAGCCHGGTAACCTTCACC | |
| | | | | | ATGGCAGGTAAGAATATTAACTTTCACCAGGCATG | |
| | | | | | GTGACTCACACCTATAATTCTAGTATATTGGGAGGC | |
| | | | | | CAAGGTGGGTGGATAACTTGAGGTCAGGAGTTCAA | |
| | | | | | GACTAGCCTGGCCAACATGGTGAATTCCCATCTCTA | |
| | | | | | ATAAAAATGCAAAAAAAAAAAAAAGCCAGAAACTG | |
| | | | | | CTTGAACCCGAGAGGTAGAGGTTGCAGTAAGCTGA | |
| | | | | | GATTGTGCCACTGCA | |
| 74 | | Ac024191 | 74 | 109 | ATGGACGGCAACGACAACGTGACCCTGCTCTTCGC | MDGNDNVTLLFAPLLRDNYTLAPNASSLGPGTNLAL |
| | | | | | CCCTCTGCTGCGGGACAACTACACCCTGGCGCCCAA | APASSAGPALGSASGRYRASASARPHSDPGAHDQRPR |
| | | | | | TGCCAGCAGCCTGGGCCCCGGCACGAACCTCGCCC | GRRGEPRPFPVPSALGAPRAPVLGHAAEPRAERVRGR |
| | | | | | TCGCCCCTGCCTCCAGCGCCGGCCCCGCCCTGGGCT | RLCITMLGLGCTVDVNHFGAHVRRPVAALLAALPVR |
| | | | | | CAGCCTCGGGCCGGTACCGAGCTTCGGCTTCAGCCC | PPAAAGLPAGPRLQAGRGGRRGLLLCGCCPGGNLSNL |
| | | | | | GGCCCCACTCCGACCCCGGAGCCCACGACCAGCGG | MSLLVDGDMNLRRAALLALSSDVGSAQTSTPGLAVS |
| | | | | | CCTCGCGGGCGGCGCGGCGAGCCACGGCCCTTCCC | PFHLYSTYKKKVSWLFDSKLVLISAHSLFCSIIMTISST |
| | | | | | CGTTCCCTCGGCCCTGGGCGCCCCACGCGCTCCCGT | LLALVLMPLCLWIYSWAWINTPTVQLLPLGTVTLTLCS |
| | | | | | TCTGGGACACGCCGCTGAACCACGGGCTGAACGTG | TLIPIGLGVFIRYKYSRVADYIVKVSLWSLLVTLVVLFI |
| | | | | | TTCGTGGGCGCCGCCTGTGCATCACCATGCTGGGCC | MTGTMLGPELLASIPAAVYVIAIFMPLAAYASGYGLA |
| | | | | | TGGGCTGCACGGTGGACGTGAACCACTTTCGGGGCG | TLFHLPPNCKRTVCLETGSQNVQLCTAILKLAFPPQFI |
| | | | | | CACGTCCGTCGGCCCGTGGCGGCGCTGCTGGCAGCT | GSMYMFPLLYALFQSAEAGIFVLIYKMYGSEMLHKR |
| | | | | | CTGCCAGTTCGGCCTCCTGCCGCTGCTGGCCTTCCT | DPLDEDEDTDISYKKLKEEEMADTSYGTVKAENIIMM |
| | | | | | GCTGGCCCTCGCCTTCAAGCTGGACGAGGTGGCCG | ETAQTSL |
| | | | | | CCGTGGGCTGCTCCTGTGTGGCTGCTGTCCCGGCGG | |
| | | | | | CAATCTCTCCAATCTTATGTCCCTGCTGGTTGACGG | |
| | | | | | CGACATGAACCTCAGACGTGCTGCTCTCTTGGCACT | |
| | | | | | CTCCTCGGATGTAGGTTCTGCCCAGACTTCAACCCC | |
| | | | | | GGGACTTGCAGTCTCCCCGTTCCACCTCTACTCAAC | |
| | | | | | ATACAAGAAAAAGGTTAGCTGGCTGTTTGACTCAA | |
| | | | | | AGCTCGTTCTGATTTCTGCACATTCCCTTTTCTGCAG | |
| | | | | | CATCATCATGACCATCTCCTCCACGCTTCTGGCCCT | |
| | | | | | CGTCTTGATGCCCCTGTGCCTGTGGATCTACAGCTG | |
| | | | | | GGCTTGGATCAACACCCCTATCGTGCAGTTACTACC | |
| | | | | | CCTAGGGACCGTGACCCTGACTCTCTGCAGCACTCT | |
| | | | | | CATACCTATCGGGTTGGGCGTCTTCATTCGCTACAA | |
| | | | | | ATACAGCCGGGTGGCTGACTACATTGTGAAGGTTTC | |
| | | | | | CCTGTGGTCTCTGCTAGTGACTCTGGTGGTCCTTTTC | |
| | | | | | ATAATGACCGGCACTATGTTAGGACCTGAACTGCTG | |
| | | | | | GCAAGTATCCCTGCAGCTGTTTATGTGATAGCAATT | |
| | | | | | TTTATGCCTTTGGCAGCGTACGCTTCAGGTTATGGT | |
| | | | | | TTAGCTACTCTCTTCCATCTTCCACCCAACTGCAAG | |
| | | | | | AGGACTGTATGTCTGGAAACAGGTAGTCAGAATGT | |
| | | | | | GCAGCTCTGTACAGCCATTCTAAAACTGGCCTTTCC | |
| | | | | | ACCGCAATTCATAGGAAGCATGTACATGTTTCCTTT | |
| | | | | | GCTGTATGCACTTTTCCAGTCTGCAGAAGCGGGGAT | |
| | | | | | TTTTGTTTTAATCTATAAAATGTATGGAAGTGAAAT | |
| | | | | | GTTGCACAAGCGAGATCCTCTAGATGAAGATGAAG | |
| | | | | | ATACAGATATTTCTTATAAAAAACTAAAAGAAGAG | |
| | | | | | GAAATGGCAGACACTTCCTATGGCACAGTGAAAGC | |
| | | | | | AGAAAATATAATAATGATGGAAACCGCTCAGACTT | |
| | | | | | CTCTCTAAATGTAATAATGATGGAAACCGCTCAGAC | |
| | | | | | TTCTCTCTAAATGTGGAGATACACAGGAGCTCTTAT | |
| | | | | | CTTGCTGAAATATTGCTTCATATTTATAGCCTGTGG | |
| | | | | | TAGTGCACATGGTTAACATAAAAGATAACACTGGT | |
| | | | | | TCACATCATACATGTAACAATTCTGATCTTTTTAAG | |
| | | | | | GTTCACTGGTGTATTAACCAAACGTTGTCACAAATT | |
| | | | | | ACAAATCAATGCTGTAATATAATTTGCACCTGGAAT | |
| | | | | | GGCTAACGTGAAGCCTGAATTAAATGTGGTTTTTAG | |
| | | | | | TTTTTACCATCACCAATTTCTATGACTGTTGCAAAT | |
| | | | | | ACAGAATCTATTTAGAAAAC | |
| 74 | | Ac024191 | 74 | 284 | ATGGACGGCAACGACAACGTGACCCTGCTCTTCGC | MDGNDNVTLLFAPLLRDNYTLAPNASSLGPGTNLAL |
| | | | | | CCCTCTGCTGCGGGACAACTACACCCTGGCGCCCAA | APASSAGPALGSASGRYRASASARPHSDPGAHDQRPR |
| | | | | | TGCCAGCAGCCTGGGCCCCGGCACGGACCTCGCCC | GRRGEPRPFPVPSALGAPRAPVLGHAAEPRAERVRGR |
| | | | | | TCGCCCCTGCCTCCAGCGCCGGCCCCGGCCCTGGGC | RLCITMLGLGCTVDVNHFGAHVRRPVAALLAALPVR |
| | | | | | TCAGCCTCGGGCCGGGTCCGAGCTTCGGCTTTCAGCC | PPAAAGLPAGPRLQAGRGGRRGLLLCGCCPGGNLSNL |
| | | | | | CCGGCCCCACTCCGACCCCGGAGCCCACGACCAGC | MSLLVDGMNLRRAALLALSSDVGSAQTSTPGLAVS |
| | | | | | GGCCTCGCGGGCGGCGCGGCGAGCCACGGCCCTTC | PFHLYSTYKKKVSWLFDSKLVLISAHSLFCSIIMTISST |
| | | | | | CCCGTTTCCCTCGGCCCTGGGCGCCCCACGCGCTCCC | LLALVLMPLCLWIYSWAWINTPIVQLLPLGTVTLTLCS |
| | | | | | GTTCTGGGACACGCCGCTGAACCACGGGCTGAACG | TLIPIGLGVFIRYKYSRVADYIVKVSLWSLLVTLVVLFI |
| | | | | | TGTTCGTGGGCGCCGCCCTGTGCATCACCATGCTGG | MTGTMLGPELLASIPAAVYVIAIFMPLAAYASGYGLA |
| | | | | | GCCTGGGCTGCACGGTGGACGTGAACCACTTCGGG | TLFHLPPNCKRTVCLETGSQNVQLCTAILKLAFPPQFI |
| | | | | | GCGCACGTCCGTCGGCCCGTGGGCGCGCTGCTGGC | GSMYMFPLLYALFQSAEAGIFVLIYKMYGSEMLHKR |
| | | | | | AGCGCTCTGCCAGTTCGGCCTCCTGCCGCTGCTGGC | DPLDEDEDTDISYKKLKEEEMADTSYGTVKAENIIMM |
| | | | | | CTTCCTGCTGGCCCTCGCCTTCAAGCTGGACGAGGT | ETAQTSL |
| | | | | | GGCCGCCGTGGCGGTGCTCCTGTGTGGCTGCTGTCC | |
| | | | | | CGGCGGCAATCTCTCCAATCTTATGTCCCTGCTGGT | |
| | | | | | TGACGGCGACATGAACCTCAGACGTGCTGCTCTCTT | |
| | | | | | GGCACTCTCCTCGGATGTAGGTTCTGCCCAGACTTC | |
| | | | | | AACCCCGGGACTTGCAGTCTCCCCGTTCCACCTCTA | |
| | | | | | CTCAACATACAAGAAAAAGGTTAGCTGGCTGTTTG | |
| | | | | | ACTCAAAGCTCGTTCTGATTTCTGCACATTCCCTTTT | |
| | | | | | CTGCAGCATCATCATGACCATCTCCTCCACGCTTCT | |
| | | | | | GGCCCTCGTCTTGATGCCCCTGTGCCTGTGGATCTA | |
| | | | | | CAGCTGGGCTTGGATCAACACCCCTATCGTGCAGTT | |
| | | | | | ACTACCCCTAGGGACCGTGACCCTGACTCTCTGCAG | |
| | | | | | CACTCTCATACCTATCGGGTTGGGCGTCTTCATTCG | |
| | | | | | CTACAAATACAGCCGGGTGGCTGACTACATTGTGA | |
| | | | | | AGGTTTCCCTGTGGTCTCTGCTAGTGACTCTGGTGG | |
| | | | | | TCCTTTTCATAATGACCGGCACTATGTTAGGACCTG | |
| | | | | | AACTGCTGGCAAGTATCCCTGCAGCTGTTTATGTGA | |
| | | | | | TAGCAATTTTTATGCCTTTGGCAGGCTACGCTTCAG | |
| | | | | | GTTATGGTTTAGCTACTCTCTTCCATCTTCCACCCAA | |
| | | | | | CTGCAAGAGGACTGTATGTCTGGAAACAGGTAGTC | |
| | | | | | AGAATGTGCAGCTCTGTACAGCCATTCTAAAACTGG | |
| | | | | | CCTTTCCACCGCAATTCATAGGAAGCATGTACATGT | |
| | | | | | TTCCTTTGCTGTATGCACTTTTCCAGTCTGCAGAAG | |
| | | | | | CGGGGATTTTTTGTTTTAATCTATAAAATGTATGGAA | |
| | | | | | GTGAAATGTTGCACAAGCGAGATCCTCTAGATGAA | |
| | | | | | GATGAAGATACAGATATTTCTTATAAAAAACTAAA | |
| | | | | | AGAAGAGGAAATGGCAGACACTTCCTATGGCACAG | |
| | | | | | TGAAAGCAGAAAATATAATAATGATGGAAACCGCT | |
| | | | | | CAGACTTCTCTCTAAATGTGGAGATACACAGGAGCT | |
| | | | | | TCTATCTTGCTGAAATATTGCTTCATATTTATAGCCT | |
| | | | | | GTGGTAGTGCACATGGTTAACATAAAAGATAACAC | |
| | | | | | TGGTTCACATCATACATGTAACAATTCTGATCTTTTT | |
| | | | | | AAGGTTCACTGGTGTATTAACCAAACGTTGTCACAA | |
| | | | | | ATTACAAATCAATGCTGTAATATAATTTGCACCTGG | |
| | | | | | AATGGCTAACGTGAAGCCTGAATTAAATGTGGTTTT | |
| | | | | | TAGTTTTTACCATCACCAATTTCTATGACTGTTGCA | |
| | | | | | AATACAGAATCTATTAGAAAAC | |
| 75 | | Ac022137 | 75 | 110 | GAGCAGATTCGCACAAACCCGGAAGCGGGTCGCGT | MMKEFSSTAQGNTEVIHTGTLQRHESHHIRDFCFQEIE |
| | | | | | GGAGTGACGGTCCCACCGCGGGGATATCTCTTCCA | KDIHNFEFQWQEEERNGHEAPMTEIKELTGSTDRHDQ |
| | | | | | AATGCATGATGAAGGAGTTCTCATCCACAGCGCAA | RHAGNKPIKDQLGSSFHSHLPELHIFQPEWKIGNQVEK |
| | | | | | GGCAATACAGAAGTGATCCACACAGGGACATTGCA | SIINASLILTSQRISCSPKTRISNNYGNNSLHSSLPIQKL |
| | | | | | AAGACATGAAAGTCATCACATTAGAGATTTTTGCTT | |
| | | | | | CCAGGAAATTGAGAAAGATATTCATAACTTTGAGTT | |
| | | | | | TCAGTGGCAAGAAGAGGAAAGGAATGGTCACGAA | |
| | | | | | GCACCCATGACAGAAATCAAAGAGTTGACTGGTAG | |
| | | | | | TACAGACCGACATGATCAAAGGCATGCTGGAAACA | |
| | | | | | AGCCTATTAAAGATCAGCTTGGATCCAGCTTTCATT | |
| | | | | | CGCATCTGCCTGAACTCCACATATTTCAGCCTGAAT | |
| | | | | | GGAAAATTGGTAATCAAGTTGAGAAGTCTATCATC | |
| | | | | | AATGCCTCCTTAATTTTGACATCCCAAAGAATTTCT | |
| | | | | | TGTAGTCCCAAAACCCGTATTTCTAATAACTATGGG | |
| | | | | | AATAATTCCCTCCATTCTTCATTACCCATACAAAAA | |
| | | | | | TTGG | |
| 76 | | Ac005027 | 76 | 111 | CTTTCCAGCCGCGGCCGACGCACCCCGGCCGCCGCC | MSGSSGTPYLGSKISLISKAQIRYEGILYTIDTDNSTVA |
| | | | | | ATGAGCGGCTCCTCAGGCACCCCGTATCTGGGCAG | LAKVRSFGTEDRPTDRPAPPREEIYEYIIFRGSDIKDITV |
| | | | | | CAAGATCAGCCTCATCTCCAAGGCGCAGATCCGCT | CEPKAQHTLPQDPAIVQSSLGSASASPFQPHVPYSPF |
| | | | | | ACGAGGGCATTCTCTACACCATCGACACCGACAAC | RGMAPYGPLAASSLLSQQYAASLGLGAGFPSIPVGKS |
| | | | | | TCCACCGTGGCGCTCGCCAAAGTGAGGTCCTTTGGC | PMVEQAVQTGSADNLNAKKLLPGKGTTGTQLNGRQ |
| | | | | | ACTGAAGACCGTCCCACAGATAGGCCTGCGCCCCC | AQPSSKTASDVVQPAAVQAQGQVNDENRRPQRRRSG |
| | | | | | CAGAGAGGAGATTTATGAGTACATCATTTTCCGAG | NRRTRNRSRGQNRPTNVKENTIKFEGDFDFESANAQF |
| | | | | | GAAGTGACATCAAGGATATCACTGTGTGTGAACCT | NREELDKETKKKLNTKDDKAEKGEEKDLAVVTQSAE |
| | | | | | CCGAAAGCTCAGCACACACTCCCGCAGGATCCCGC | APAEEDLLGPNCYYDKSKSFFDNISSELKTSSRRTTWA |
| | | | | | CATTGTTCAGTCTTCCCTGGGTTTCTGCCTCCGCCTCG | EERKLNTETFGVSGRFLRGRSSRGGFRGGRGNGTTRR |
| | | | | | CCCTTCCAGCCGCACGTGCCTTACAGCCCTTTCCGA | NPTSHRAGTGRV |
| | | | | | GGGATGGCGCCCTACGGCCCGCTGGCGGCCAGCTC | |
| | | | | | CCTGCTCAGCCAGCAGTATGCCGCCTCCCTGGGTCT | |
| | | | | | AGGAGCTGGTTTTCCATCCATCCCAGTCGGCAAGAG | |
| | | | | | CCCCATGGTGGAGCAGGCTGTGCAGACTGGTTCTGC | |
| | | | | | TGACAACCTGAATGCTAAAAAGCTGTTACCTGGCA | |
| | | | | | AGGGCACCACAGGGACGCAGCTCAACGGTCGTCAG | |
| | | | | | GCCCAGCCGAGCAGCAAGACGGCCAGCGATGTAGT | |
| | | | | | CCAGCCGGCAGCTGTGCAAGCTCAAGGGCAGGTGA | |
| | | | | | ATGACGAGAACAGAAGACCTCAGAGGAGGCGATCA | |
| | | | | | GGAAACAGGCGAACAAGGAATCGCTCCAGAGGGC | |
| | | | | | AAAACCGTCCAACTAACGTTAAGGAAAACACAATC | |
| | | | | | AAATTTGAGGGTGACTTTGATTTCGAGAGTGCAAAT | |
| | | | | | GCCCAGTTCAACCGAGAGGAGCTTGACAAAGAATT | |
| | | | | | TAAGAAGAAACTGAATTTTAAAGATGACAAGGCTG | |
| | | | | | AGAAGGGGGAAGAGAAGGACCTGGCTGTGGTGACC | |
| | | | | | CAGAGTGCCGAAGCGCCCGCTGAGGAAGACCTTCT | |
| | | | | | GGGGCCCAACTGCTACTATGACAAATCCAAGTCGTT | |
| | | | | | CTTCGACAACATCTCTTCTGAACTCAAGACCAGCTC | |
| | | | | | CAGGCGGACGACGTGGGCCGAAGAGAGGAAGCTC | |
| | | | | | AACACAGAGACCTTTGGGGTGTCAGGGAGGTTTCTT | |
| | | | | | CGTGGCCGCAGTTCTCGGGGCGGATTCCGAGGAGG | |
| | | | | | CAGGGGCAATGGGACCACCCGTCGCAACCCCACTT | |
| | | | | | CCCACAGGGCCGGGACTGGCAGGGTGTGAGGGTGC | |
| | | | | | AGCCAAAGGCTCCTACTGAAGTGGCGCATAACTGA | |
| | | | | | CGCTGTGTGTGTCAGGACGCGAGGAAAACGCTGCA | |
| | | | | | CTTACAGGGAGAGGTGGTCACTTTGTTACGGAGTT | |
| | | | | | TGGAAGAGACCCATACTGCTACGTTGTTTTGGACT | |
| | | | | | TAACTGAACTTGGACATGGTCTGAGTTAGAACCACT | |
| | | | | | TGTTTTGGGGAAGTATTCATGGGTAACCTCTTTGAG | |
| | | | | | GTCTCTTTATCTGTGTTTCCTTTTTAGTTGCGCATAG | |
| | | | | | CCTAATTCTAAGGTTTTGGTATTTTGCAAAAAGGTT | |
| | | | | | TCTATAGTGAAAGCTGAATCCTTACTTTGTGACTTT | |
| | | | | | TTTTTTTTTTTTTAATGACAAGCTTTGACTTTTAAAA | |
| | | | | | GTGGAACCAAATCTGTTGGCAGAGGTGGCAGCCAA | |
| | | | | | GTACATCTCTGTAACCCAGCTGGCCCCTGGTGCTGT | |
| | | | | | TGGCCTGGCACCCCACTGCCAAGGGTGGGGTCTCA | |
| | | | | | GGAGTCAGGCAGGGCCAGCACAGGGTGGCGTGGGG | |
| | | | | | GGCAGGGGTGGGTGGGTGGAGGGCACGGAAGGGG | |
| | | | | | TTTTCCCATGGATCATGTTGTATAAGTGAACCAGAC | |
| | | | | | CACCCTGATGGCATCCACAGTGATGTCAAGGTTGG | |
| | | | | | GGCTGGCCAGGGGTGGGTGGACTAGAAGCATTTGG | |
| | | | | | GAGTAGTGGCCAGGGGCCCTGGACGCTAGCCACGG | |
| | | | | | AGCTGCTGCACAGAGCCTGGTGTCCACAAGCTTCCA | |
| | | | | | GGTTGGGGTTGGAGCCTGGGATGAGCCCCGGCAGC | |
| | | | | | GCCTTGGCCCTTCTGTGGTCCCTGCCAGCCTCTGAC | |
| | | | | | CTGGGCCGGTCAGTCATTGCTGGACTCTGGCCACAC | |
| | | | | | ACTGGCGTTCTCATCCACTTGGAAACAAGCCAGTCT | |
| | | | | | TTTCTGCAAGGTCAGTTGACCAAGAGCATATTTCCC | |
| | | | | | CTCTGTTGTACATCGTTGTTTTGTGTTTGTGTTGTAA | |
| | | | | | CAGTGGGTGGAGGGAGGGTGGGGTCTACATTTGTT | |
| | | | | | GCATGAGTCGATGGGTCAGAACTTTAGTATACGCAT | |
| | | | | | GCGTCCTCTGAGTGACAGGGCATTTTGTCGAAAATA | |
| | | | | | AGCACCTTGGTAACTAAACCCCTCTAATAGCTATAA | |
| | | | | | AGGCTTTAGTTCTGTATTGATTAAGTTACTGTAAAA | |
| | | | | | GCTTGGGTTTATTTTTGTAGGACTTAATGGCTAAGA | |
| | | | | | ATTAGAACATAGCAAGGGGGCTCCTCTGTTGGAGT | |
| | | | | | AATGTAAATTGTAATTATAAATAAACATGCAAACCT | |
| | | | | | TTAAAATTTTTCTTTTTCTGATGCTCTAAGAATCCTGT | |
| 77 | | Ac022694 | 77 | 112 | GGGCGGTTGTGACGTTGCTAGCGCTTGTCCGGTGGC | MALPKDAIPSLSECQCGICMEILVEPVTLPCNHTLCKP |
| | | | | | TGCTGCGCTGCCGCAACGAATAGGGTTTCTGGCTGC | CFQSTVEKASLCCPFCRRVSSWTRYHTRRNSLVNVEL |
| | | | | | GTAGGAGGGACGGGGGCGCGGAGCTCTGGGAAACT | WTIIQKHYPRECKLRASGQESEEVGDDYQPVRLLSKP |
| | | | | | GCGCCAGGCGCCCGAAAGGTGAACACGGGAGTCGC | GELRREYEEEISKVAAERRASEEEENKASEEYIQRLLA |
| | | | | | GCGTCTCCCCCGCAGCAGCGGTAAAGCGGAAGTTA | EEEEEEKRQAEKRRRAMEEQLKSDEELARKLSINNFC |
| | | | | | TGCTGCAGCCGGAGCCCGGGCTTCCTCCCGGAGCC | EGSISASPLNSRKSDPVTPKSEKKSKNKQRNTGDIQKY |
| | | | | | GCGTCCCGGGGCCCGGCTGCCCCGAGCTGAGCGGA | LTPKSQFGSASHSEAVQEVRKDSVSKDIDSSKRKSPTG |
| | | | | | GCATCCTTTCCGGGTGAGGGGAGGAGAGGACTTGG | QDTEIEDMPTLSPQISLGVGEQGADSSIESPMPWLCAC |
| | | | | | CTCGTTCCCCTCGCTGCCCCGGGAGGCCGCAGCCGCG | GAEWYHEGNVKTRPSNHGKELCVLSHERPKTRVPYS |
| | | | | | GTGTTCATGCCGCGGAGCAGCCAGGCTCCTCCGAC | KETAVMPCGRTESGCAPTSGVTQTNGNNTGETENEES |
| | | | | | GAAAACCTGCATTTATTTGCTGGCGGGACGTTTGCC | CLLISKEISKRKNQESSFEAVKDQCFSAKRRKVSPESSP |
| | | | | | TTGAAAATGGACAAAGACGCCGCCCTCCGGGTAT | DQEETEINFTQKLIDLEHLLFERHKQEEQDRLLALQLQ |
| | | | | | TCCTGTTTGCCTGACCCTGAGAGCGCCTTTTTGCTTC | KEVDKEQMVPNRQKGSPDEYHLRATSSPPDKVLNGQ |
| | | | | | AAGACGTGTTGGATGCTCCTGTTCTCCGAATTCTGA | RKNPKDGNFKRQTHTKHPTPERGSRDKNRQVSLKMQ |
| | | | | | TACGCTTCTGGGCATAATACTGAAACACAAAACTG | LKQSVNRRKMPNSTRDHCKVSKSAHSLQPSISQKSVF |
| | | | | | CTTTTGCTCTCTCTGTGGTTGGCCGAAAATAGGATT | QMTQRCTK |
| | | | | | CTTTTCGTGCAGGTGTCGTTGTTTAGTCGGCTTTAC | |
| | | | | | TAACATATTGAAATGGCTCTACCCAAAGACGCCATC | |
| | | | | | CCCTCGCTGTCCGAGTGCCAGTGCGGGATCTGCATG | |
| | | | | | GAAATCCTCGTGGAGCCCGTCACCCTCCCGTGTAAC | |
| | | | | | CACACGCTGTGTAAACCGTGCTTCCAGTCGACCGTC | |
| | | | | | GAAAAGGCGAGTTTATGCTGTCCCTTCTGTCGCCGC | |
| | | | | | CGGGTATCGTCGTGGACTCGGTACCATACCCGAAG | |
| | | | | | AAATTCTCTCGTCAACGTGGAACTGTGGACGATAAT | |
| | | | | | TCAAAAACACTATCCCAGGGAGTGCAAGCTTAGAG | |
| | | | | | CGTCTGGCCAAGAATCAGAGGAAGTGGGTGATGAC | |
| | | | | | TATCAGCCAGTTCGTCTGCTCAGTAAACCTGGGGAA | |
| | | | | | CTGAGAAGAGAATATGAAGAGGAAATAAGCAAGG | |
| | | | | | TGGCGGCAGAGCGACGGGCCAGCGAGGAAGAAGA | |
| | | | | | AAACAAAGCCAGTGAAGAATACATACAGAGGTTGT | |
| | | | | | TGGCAGAGGAGGAAGAAGAGGAAAAAAGACAGGC | |
| | | | | | AGAAAAAAGGCGAAGAGCGATGGAAGAACAACTG | |
| | | | | | AAAAGTGATGAGGAACTGGCAAGAAAGCTAAGCAT | |
| | | | | | TAACAATTTCTGTGAGGGAAGTATCTCGGCTCTCC | |
| | | | | | CTTGAATTCCAGAAAATCTGATCCAGTTACACCCAA | |
| | | | | | GTCTGAAAAGAAAAGTAAGAACAAACAAAGAAAC | |
| | | | | | ACTGGAGATATTCAGAAGTATTTGACACCGAAATCT | |
| | | | | | CAGTTTGGGTCAGCCTCACACTCTGAAGCTGTACAA | |
| | | | | | GAAGTCAGGAAAGACTCCGTATCTAAGGACATTGA | |
| | | | | | CAGTAGTGATAGGAAAAGCCCAACAGGGCAAGACA | |
| | | | | | CAGAAATAGAAGATATGCCGACACTTTCTCCACAG | |
| | | | | | ATATCCCTTGGAGTTGGAGAACAAGGTGCAGATTCT | |
| | | | | | TCAATAGAGTCCCCTATGCCATGGTTATGTGCCTGT | |
| | | | | | GGTGCCGAATGGTACCATGAAGGAAACGTCAAAAC | |
| | | | | | AAGACCAAGCAATCATGGGAAAGAGTTATGTGTCT | |
| | | | | | TAAGTCACGAGCGACCTAAAACCAGAGTTCCCTAC | |
| | | | | | TCGAAAGAAACTGCAGTTATGCCTTGTGGCAGAAC | |
| | | | | | AGAAAGTGGGTGCGCCCCCACATCAGGGGTGACAC | |
| | | | | | AGACAAATGGAAACAACACAGGTGAGACAGAAAA | |
| | | | | | TGAAGAGTCGTGCCTACTGATCAGTAAGGAGATTTC | |
| | | | | | CAAAAGAAAAAACCAAGAATCTTCCTTTGAAGCAG | |
| | | | | | TCAAGGATCAATGCTTTTCTGCAAAAAGAAGAAAA | |
| | | | | | GTGTCCCCCGAATCTTCCCCAGATCAAGAGGAAAC | |
| | | | | | AGAAATAAACTTTACCCAAAAACTGATAGATTTGG | |
| | | | | | AGCATCTACTGTTTGAGAGACATAAACAAGAAGAA | |
| | | | | | CAGGACAGGTTATTGGCATTACAACTTCAGAAGGA | |
| | | | | | GGTGGATAAAGAGCAAATGGTGCCAAACCGGCAAA | |
| | | | | | AAGGATCCCCAGATGAGTATCACTACGCGCTACAT | |
| | | | | | CCTCCCCTCCAGACAAAGTGCTAAATGGACAGAGG | |
| | | | | | AAGAATCCCAAAGATGGGAACTTCAAAAGGCAAAC | |
| | | | | | TCACACAAAGCATCCAACACCAGAGAGAGGCTCAA | |
| | | | | | GGGACAAAAATAGGCAAGTGTCTTTAAAGATGCAG | |
| | | | | | TTGAAGCAGTCAGTTAATAGAAGAAAGATGCCAAA | |
| | | | | | TCTACTAGAGATCACTGTAAGGTATCCAAAAGTGC | |
| | | | | | TCACTCCCTACAGCCTAGCATTTCACAGAAAAGTGT | |
| | | | | | TTTTCAGATGTTTCAGAGATGCACAAAGTAAGGCCT | |
| | | | | | GGTAAAGGGAGTGCTTTGTGATCTAGTAAAGCTGG | |
| | | | | | AATGTGAAGCTCTTTCCTAAAAAAAAAA | |
| 78 | 235347 | | 78 | 113 | CCGTGACCTCCATGTGGGAGCTCCAGCTCTATAAGT | MWIQVRTIDGSKTCTIEDVSRKATIEELRERVWALFD |
| | | | | | AAACACTCTGCGCGGCGCAGACATGGCCTCTTCCTA | VRPECQRLTYRGKQLENGYTLTDYDVGLNDTTQLLVR |
| | | | | | TCTTTGAGGCGGTGTCTGCGGCAGCGCCTCAGAGTG | PDPDHLPGTSTQIEAKPCSNSPPKVKKAPRVGPSNQPS |
| | | | | | GTTCCGGTCGTCTCTCCTCAAGTCGGCTAGTCGGGC | TSARARLIDPGFGIYKVNELVDARDVGLGAWFEAHIH |
| | | | | | GCGCGCGCTGAGAGTCGTCGCCGCCTGTCGGGCCC | SVTRASDGQSRGKTPLKNGSSCKRTNGNTKHKSKENT |
| | | | | | GGCGTCCGGTCGGTCCGGTGGGCGCGCTCGCCCGC | NKLDSVPSTSNSDCVAADEDVTYHTQYDEYPESGTLE |
| | | | | | CTGCCGCTGAGGGCCCGAGCCGCAGGGAAAGCGGC | MNVKDLRPRARTILKWNELNVGDVVMVNYNVESPG |
| | | | | | GCGGGCCGGGCGGGGCGCGGCGCCCAGAGCTCAGG | QRGTWTDAETTTLKTTSRTKKELRVKTTLGGSEGTLND |
| | | | | | GGGAGACAAAGGGGACCGGTTCCTCTCTAGGCGCC | CKTTSVDETTKTERPGAHPLSTADGKTLRRNDPECDLCG |
| | | | | | AAGATGTGGATACAGGTTCGCACCATTGATGGCTCC | GDPEKKCHSCSCRVCGGKHEPNMQLLCDECNVAYHI |
| | | | | | AAGACGTGCACCATTGAGGACGTGTCTCGCAAAGC | YCLNPPLDKVPEEEYWYCPSCKTDSSEVVKAGERLK |
| | | | | | CACGATGAGGAGCTGCGCGAGCGGGTGTGGGCGC | MSKKKAKMPSASTESRRDWGRGMACVGRTRECTIVP |
| | | | | | TGTTCGACGTGCGGCCCGAATGCCAGCGCCTCTTCT | SNHYGPIPGIPVGSTWRFRVQVSEAGVHRPHVGGIHG |
| | | | | | ACCGGGCAAGCAGTTGGAAAATGGATATACCTTA | RSNDGAYSLVLAGGFADEVDRGDEFTYTGSGGKNLA |
| | | | | | TTTGATTATGATGTTGGACTGAATGATATAATTCAG | GNKRIGAPSADQTLTNMNRALALNCDAPLDDKIGAES |
| | | | | | CTGCTAGTTCGCCCAGACCCTGATCATCTTCCTGGC | RNWRAGKPVRVIRSFKGRKISKYAPEEGNRYDGIYKV |
| | | | | | ACATCTACACAGATTGAGGCTAAACCCTGTTCTAAT | VKYWPEISSSHGFLVWRYLLRRDDVEPAPWTSEGIER |
| | | | | | AGTCCACCTAAAGTAAAGAAAGCTCCGAGGGTAGG | SRRLCLRGLCLGKVGPVN |
| | | | | | ACCTTCCAATCAGCCATCTACATCAGCTCGTGCCCG | |
| | | | | | TCTTATTGATCCTGGCTTTGGAATATATAAGGTAAA | |
| | | | | | TGAATTGGTGGATGCCAGAGATGTCGGCCTTGGTGC | |
| | | | | | TTGGTTTGAAGCACACATACATAGTGTTACTAGAGC | |
| | | | | | TTCTGATGGACAGTCACGTGGCAAACTCCACTGA | |
| | | | | | AGAATGGCAGTTCTTGTAAAAGGACTAATGGAAAT | |
| | | | | | ATAAAGCATAAATCCAAAGAGAACACAAATAAATT | |
| | | | | | GGACAGTGTACCCTCTACGTCTAATTCAGACTGTGT | |
| | | | | | TGCTGCTGATGAAGACGTTATTTACCATATCCAGTA | |
| | | | | | TGATGAATACCCAGAAAGCGGTACTCTAGAAATGA | |
| | | | | | ATGTCAAGGATCTTAGACCACGCTAGAACCATTT | |
| | | | | | TGAAATGGAATGAACTAAATGTTGGTGATGTGGTA | |
| | | | | | ATGGTTAATTATAATGTAGAAAGTCCTGGACAAAG | |
| | | | | | AGGATTCTGGTTTGATGCAGAAATTACCACATTGAA | |
| | | | | | GACAATCTCAAGGACCAAAAAAGAACTTCGTGTGA | |
| | | | | | AAATTTTCCTGGGGGGTTCTGAAGGAACATTAAATG | |
| | | | | | ACTGCAAGATAATATCTGTAGATGAAATCTTCAAG | |
| | | | | | ATTGAGAGACCTGGAGCCCATCCCCTTTCATTTGCA | |
| | | | | | GATGGAAAGTTTTTAAGGCGAAATGACCCTGAATG | |
| | | | | | TGACCTGTGTGGTGGAGACCCAGAAAAGAAATGTC | |
| | | | | | ATTCTTGCTCCTGTCGTGTATGTGGTGGGAAACATG | |
| | | | | | AACCCAACATGCAGCTTCTGTGTGATGAATGTAATG | |
| | | | | | TGGCTTATCATATTTACTGTCTGAATCCACCTTTGG | |
| | | | | | ATAAAGTCCCAGAAGAGGAATACTGGTATTGTCCTT | |
| | | | | | CTTGTAAAACTGATTCCAGTGAAGTTGTAAAGGCTG | |
| | | | | | GTGAAAGACTCAAGATGAGTAAAAAGAAAGCAAA | |
| | | | | | GATGCCGTCAGCTAGTACTGAAAGCCGAAGAGACT | |
| | | | | | GGGGCAGGGGAATGGCTTGTGTTGGTCGTACGAGA | |
| | | | | | GAATGTACTATTGTCCCTTCTAATCATTATGGACCC | |
| | | | | | ATTCCTGGTATTCCTGTTGGATCAACTTGGAGATTT | |
| | | | | | AGAGTTCAGGTGAGCGAAGCAGGTGTTCACAGACC | |
| | | | | | CCATGTTGGTGGAATTCATGGTCGAAGTAATGATGG | |
| | | | | | GGCTTATTCTCTTGTACTGGCTGGTGGATTTGCGGA | |
| | | | | | TGAAGTCGACCGAGGTGATGAGTTCACATACACTG | |
| | | | | | GAAGCGGTGGTAAAAATCTTGCTGGTAACAAAAGA | |
| | | | | | ATTGGTGCACCTTCAGCTGATCAAACATTAACAAAC | |
| | | | | | ATGAACAGGGCATTGGCCCTAAACTGTGATGCTCC | |
| | | | | | ATTGGATGATAAAATTGGAGCAGAGTCTCGGAATT | |
| | | | | | GGAGAGCTGGTAAGCCAGTCAGAGTGATACGCAGT | |
| | | | | | TTTAAAGGGAGGAAGATCAGCAAATATGCTCCTGA | |
| | | | | | AGAAGGCAACAGATATGATGGCTTTATAAGGTGG | |
| | | | | | TGAAATACTGGCCAGAGATTTCATCAAGCCATGGA | |
| | | | | | TTCTTGGTTTGGCGCTATCTTTTAAGAAGAGATGAT | |
| | | | | | GTTGAACCTGCTCCTTGGACCTCTGAAGGAATAGAA | |
| | | | | | CGGTCAAGGAGATTATGTCTACGTGGGTTGTGCTTG | |
| | | | | | GGAAAAGTTGGACCTGTTAATTAAAAGTAAAATAT | |
| | | | | | TTCCAAATCAATTTGGAAATGACTTGAAGTGTGAGG | |
| | | | | | GAAAGGGATTCATAAAATTTAGGTATAGGAGGCCC | |
| | | | | | TGGAAAAGGACATTTATCCTAGAGGGCACAGGGGG | |
| | | | | | TGTCTCTCTGGTAGGGGAAGGGTGGGGAGGTGGCT | |
| | | | | | TTATAAGAGTGGTCTGCCTTCTCCCTTTCTCACTTTT | |
| | | | | | CCTCACCCCTTTTCTCTCTTCCCCCGCAAAGCTGCTT | |
| | | | | | CCCTGCCCTGCCACCACCTTTAGTGCTTTGTCTTTTT | |
| | | | | | TCCCCTTTGCCCATGCTCAGCTGTTAACCCATAAAG | |
| | | | | | ACTTCGTTGATTTTGTGTGCATAGTGGATGGTATGG | |
| | | | | | CTGCATTAATCCCTTCACTGCCTGTATACCTAGAA | |
| | | | | | TTTGTCCCTGACACTGACTTCAGAGCATGGTTTGAG | |
| | | | | | TTCATCTCCCATCATTCCCCATTGUGTGCTTCCCGT | |
| | | | | | AAAAACTGCCAGCTTTATCATTTCCCCTGGCTCTGC | |
| | | | | | CCACACTGCATGTGTAGGGGCTGAACTATGGGCAA | |
| | | | | | GTGTCTGACCACCCAGGCAGGTGAGTGTGTGTCTTC | |
| | | | | | TAATGCAAGTCTGTTTCTGTTTTTGTTGTCTTTTTAA | |
| | | | | | ACTCATAGAATTGATTGTTGAAAATAAGGCCATCA | |
| | | | | | ACTGCTAAAACAACTACTAAAATAATTCTTTTTAAT | |
| | | | | | ATAAAAATAACTTTGTCAAATTCACTTTCAGAAGAT | |
| | | | | | TTTTCAGATGTCCCTGTTGAGAGCATTGTTCTAGAT | |
| | | | | | AGGTTATATTTGAAACTGTGAGCAGAAGCATGTGA | |
| | | | | | GCCCATCTGCTATGATGAGTAATAGTCATTGAGGCC | |
| | | | | | TGAAACATACAGTGCTTTAAGCATGACTGTTATTAC | |
| | | | | | AAAGCATGCTTCTCCCACCCCACCCACCCCCTCAAA | |
| | | | | | GAAGGTAGCCATTGAAACATAAGGATGATAGATAG | |
| | | | | | AATGTATTACTTCAAATCTAACTCTTAGCTGGTGGA | |
| | | | | | GGATTTAGTAATTTAGTTGCTTTAGGTCTTGTAAAA | |
| | | | | | GCTCCTGCCGCTAACTTTAGGAGATGAGAAGTTTGA | |
| | | | | | CCCTTAATGTTCTTGATATTTTTTTAGATCAACTCCA | |
| | | | | | CAATTTACTGTGATCCAATCCATCTGCTTTCTATCTG | |
| | | | | | TTGTGCTCTATGATTGGTTCTCATTTACCTTCATTTC | |
| | | | | | TGTATTCTACTTTCCTTAAACTTTAAGGAAATCTAA | |
| | | | | | TCACAACTCCTGAAGACTTACCTTTCTTAGATCTGA | |
| | | | | | AACTTAAGATCAGTGTATTATAAAATGGAATCTCTT | |
| | | | | | AGCAGTCACAGCTACATAAATTGGGATTTTAATAGT | |
| | | | | | TGTCTGTGCTTTGAATTCTTTTCCTTTAAATGTCTGT | |
| | | | | | TTCTTTTATGTAAAGTTTTTCAGTTTGGGGAACGTGT | |
| | | | | | AGTCTTCCCCTCCCTTTTAATTTCTCACCAGGATCTA | |
| | | | | | AACCCCCCTTCTCTGTGAAGCTTAAATCTGCATTGT | |
| | | | | | ACTCTCCCTCCTCCCCCCCCATCAGTATCCAGCAGG | |
| | | | | | TTACCCTTCAGATAAAGAAGGGAAGAAGCCTAAAG | |
| | | | | | GACAGTCAAAGAAGCAGCCCAGTGGAACCACAAAA | |
| | | | | | AGGCCAATTTCAGATGATGACTGTCCAAGTGCCTCC | |
| | | | | | AAAGTGTACAAAGCATCAGATTCAGCAGAAGCAAT | |
| | | | | | TGAGGCTTTTCAACTAACTCCTCAACAGCAACATCT | |
| | | | | | CATCAGAGAAGATTGTCAAAACCAGAAGCTGTGGG | |
| | | | | | ATGAAGTGCTTTCACATCTTGTGGAAGGACCAAATT | |
| | | | | | TTCTGAAAAAATTGGAACAATCTTTTATGTGCGTTT | |
| | | | | | GCTGTCAGGAGCTAGTTTACCAGCCTGTGACAACTG | |
| | | | | | AGTGCTCCACAATGTCTGTAAAGATTTGCCTACAGC | |
| | | | | | GCTCCTTTAAGGCACAGGTTTTCTCCTGCCCTGCTT | |
| | | | | | GCCGGCATGATCTTGGCCAGAATTACATCATGATTC | |
| | | | | | CCAATGAGATTCTGCAGACTCTACTTGACCTTTTCT | |
| | | | | | TCCCTGGCTACAGCAAAGGACGATGATCTGCCTGCT | |
| | | | | | TTCACTGTGTTGTTCATGGTGGCTTTTTGGACAATA | |
| | | | | | AAGAATCTAAAATGGGTGGGGAGGGTGGAAGAAAT | |
| | | | | | GGTGGACTGTATCTCTCACGTTCTGAAGCAGCTAAT | |
| | | | | | CCTCTTTCCCACATAGCCATCATCTTGTGTGTGTAGT | |
| | | | | | AAGAGGCCCATTTCTCAACTGTCTTTTAAATATCTA | |
| | | | | | AAGGTAGTTCCTGTAACAACTAGTTTTAATGAGTAA | |
| | | | | | AAAGTCAAAGCCTCAGCTCTAGTTGATATCCAAGTT | |
| | | | | | ATGATTTATTTTGCAACTACCTCAGGACAGAAAAGA | |
| | | | | | TTTATGGGGATTTTAAAAATCATTGAATAACTAGTT | |
| | | | | | AAATGAAATTTTAGCTACACACTGCCTCCCAAATAT | |
| | | | | | TAGTTGTGCCTGGTTCTTGTAATTTGATTTTACAGA | |
| | | | | | AAAGGAAATGACACTTGAGATCCTTGGAATGAACA | |
| | | | | | CAGCTTCTAAAGTGTGCATATACTTTTTTAACGTCT | |
| | | | | | CTTCTTCCATTACAATGTGTGTTTTGCAAGGACAGG | |
| | | | | | TTCATTTTTTTTAGCCCACTTTGTGAACTCCATTGTG | |
| | | | | | CTTTTTTCTGGTGTTTTATGCAAGTTGACTACTAATG | |
| | | | | | ACTAATGAGAACAATAATGAATGCATTGTTGCTGC | |
| | | | | | ATTAGTGTAATGTGGTGTGGTTTTGCACTTAAAATA | |
| | | | | | GGTATTCATATGCTCTACTTGTCAATGTTCATGAAA | |
| | | | | | ATCCACTTCTCTACTAGTCGAACTGCTTTCCCCCTCT | |
| | | | | | CACCAGTGGTTTTACATAAGCAAAAAAATGAGGGC | |
| | | | | | TGTGCTGACCTTTGAGAGGATTTGAAATTGCTTCAT | |
| | | | | | ATTGTGATCCTAAATTTTATATTCACTATATTCCCTA | |
| | | | | | AAGTATACCTAATAAATATTTTATGATCAG | |
| 78 | 235347 | | 78 | 282 | CCGTGACCTCCATGTGGGAGCTCCAGCTCTATAAGT | MWIQVRTIDGSKTCTIEDVSRKATIEELRERVWALFD |
| | | | | | AAACACTCTGCGCGGCGCAGACATGGCCTCTTCCTA | VRPECQRLFYRGKQLENGYTLFDYDVGLNDIIQLLVR |
| | | | | | TCTTTGAGGCGGTGTCTGCGGCAGCGCCTCAGAGTG | PDPDHLPGTSTQIEAKPCSNSPPKVKKAPRVGPSNQPS |
| | | | | | GTTCCGGTCGTCTCTCCTCAAGTCGGCTAGTCGGGC | TSARARLIDPGFGIYKVNELVDARDVGLGAWFEAHIH |
| | | | | | GCGCGCGCTGAGAGTCGTCGCCGCCTGTCGGGCCC | SVTRASDGQSRGKTPLKNGSSCKRTNGNIKHKSKENT |
| | | | | | GGCGTCCGGTCGGTCCGGTGGGCGCGCTCGCCCGC | NKLDSVPSTSNSDCVAADEDVIYTHIQYDEYPESGTLE |
| | | | | | CTGCCGCTGAGGGCCCGAGCCGCAGGGAAAGCGGC | MNVKDLRPRARTILKWNELNVGDVVMVNYNVESPG |
| | | | | | GCGGGCCGGGCGGGGCGCGGCGCCCAGAGCTCAGG | QRGFWFDAEITTLKTISRTKKELRVKIFLGGSEGTLND |
| | | | | | GGGAGACAAAGGGGACCGGTTCCTCTCTAGGCGCC | CKIISVDEIFKIERPGAHPLSFADGKFLRRNDPECDLCG |
| | | | | | AAGATGTGGATACAGGTTCGCACCATTGATGGCTCC | GDPEKKCHSCSCRVCGGKHEPNMQLLCDECNVAYHI |
| | | | | | AAGACGTGCACCATTGAGGACGTGTCTCGCAAAGC | YCLNPPLDKVPEEEYWYCPSCKTDSSEVVKAGERLK |
| | | | | | CACGATTGAGGAGCTGCGCGAGCGGGTGTGGGCGC | MSKKKAKMPSASTESRRDWGRGMACVGRTRECTIVP |
| | | | | | TGTTCGACGTGCGGCCCGAATGCCAGCGCCTCTTCT | SNHYGPIPGIPVGSTWRFRVQVSEAGVHRPHVGGIHG |
| | | | | | ACCGGGGCAAGCAGTTGGAAAATGGATATACCTTA | RSNDGAYSLVLAGGFADEVDRGDEFTYTGSGGKNLA |
| | | | | | TTTGATTATGATGTTGGACTGAATGATATAATTCAG | GNKRIGAPSADQTLTNMNRALALNCDAPLDDKIGAES |
| | | | | | CTGCTAGTTCGCCCAGACCCTGATCATCTTCCTGGC | RNWRAGKPVRVIRSFKGRKISKYAPEEGNRYDGIYKV |
| | | | | | ACATCTACACAGATTGAGGCTAAACCCTGTTCTAAT | VKYWPEISSSHGFLVWRYLLRRDDVEPAPWTSEGIER |
| | | | | | AGTCCACCTAAAGTAAAGAAAGCTCCGAGGGTAGG | SRRLCLRGLCLGKVGPVN |
| | | | | | ACCTTCCAATCAGCCATCTACATCAGCTCGTGCCCG | |
| | | | | | TCTTATTGATCCTGGCTTTGGAATATATAAGGTAAA | |
| | | | | | TGAATTGGTGGATGCCAGAGATGTCGGCCTTGGTGC | |
| | | | | | TTGGTTTGAAGCACACATACATAGTGTTACTAGAGC | |
| | | | | | TTCTGATGGACAGTCACGTGGCAAAACTCCACTGA | |
| | | | | | AGAATGGCAGTTCTTGTAAAAGGACTAATGGAAAT | |
| | | | | | ATAAAGCATAAATCCAAAGAGAACACAAATAAATT | |
| | | | | | GGACAGTGTACCCTCTACGTCTAATTCAGACTGTGT | |
| | | | | | TGCTGCTGATGAAGACGTTATTTACCATATCCAGTA | |
| | | | | | TGATGAATACCCAGAAAGCGGTACTCTAGAAATGA | |
| | | | | | ATGTCAAGGATCTTAGACCACGAGCTAGAACCATTT | |
| | | | | | TGAAATGGAATGAACTAAATGTTGGTGATGTGGTA | |
| | | | | | ATGGTTAATTATAATGTAGAAAGTCCTGGACAAAG | |
| | | | | | AGGATTCTGGTTTGATGCAGAAATTACCACATTGAA | |
| | | | | | GACAATCTCAAGGACCAAAAAAGAACTTCGTGTGA | |
| | | | | | AAATTTTCCTGGGGGGTTCTGAAGGAACATTAAATG | |
| | | | | | ACTGCAAGATAATATCTGTAGATGAAATCTTCAAG | |
| | | | | | ATTGAGAGACCTGGAGCCCATCCCCTTTCATTTGCA | |
| | | | | | GATGGAAAGTTTTTAAGGCGAAATGACCCTGAATG | |
| | | | | | TGACCTGTGTGGTGGAGACCCAGAAAAGAAATGTC | |
| | | | | | ATTCTTGCTCCTGTCGTGTATGTGGTGGGAAACATG | |
| | | | | | AACCCAACATGCAGCTTCTGTGTGATGAATGTAATG | |
| | | | | | TGGCTTATCATATTTACTGTCTGAATCCACCTTTGG | |
| | | | | | ATAAAGTCCCAGAAGAGGAATACTGGTATTGTCCTT | |
| | | | | | CTTGTAAAACTGATTCCAGTGAAGTTGTAAAGGCTG | |
| | | | | | GTGAAAGACTCAAGATGAGTAAAAAGAAAGCAAA | |
| | | | | | GATGCCGTCAGCTAGTACTGAAAGCCGAAGAGACT | |
| | | | | | GGGGCAGGGGAATGGCTTGTGTTGGTCGTACGAGA | |
| | | | | | GAATGTACTATTGTCCCTTCTAATCATATGGACCC | |
| | | | | | ATTCCTGGTATTCCTGTTGGATCAACTTGGAGATTT | |
| | | | | | AGAGTTCAGGTGAGCGAAGCAGGTGTTCACAGACC | |
| | | | | | CCATGTTGGTGGAATTCATGGTCGAAGTAATGATGG | |
| | | | | | GGCTTATTCTCTTGTACTGGCTGGTGGATTTGCGGA | |
| | | | | | TGAAGTCGACCGAGGTGATGAGTTCACATACACTG | |
| | | | | | GAAGCGGTGGTAAAAATCTTGCTGGTAACAAAAGA | |
| | | | | | ATTGGTGCACCTTCAGCTGATCAAACATTAACAAAC | |
| | | | | | ATGAACAGGGCATTGGCCCTAAACTGTGATGCTCC | |
| | | | | | ATTGGATGATAAAATTGGAGCAGAGTCTCGGAATT | |
| | | | | | GGAGAGCTGGTAAGCCAGTCAGAGTGATACGCAGT | |
| | | | | | TTTAAAGGGAGGAAGATCAGCAAATATGCTCCTGA | |
| | | | | | AGAAGGCAACAGATATGATGGCATTTATAAGGTGG | |
| | | | | | TGAAATACTGGCCAGAGATTTCATCAAGCCATGGA | |
| | | | | | TTCTTGGTTTGGCGCTATCTTTTAAGAAGAGATGAT | |
| | | | | | GTTGAACCTGCTCCTTGGACCTCTGAAGGAATAGAA | |
| | | | | | CGGTCAAGGAGATTATGTCTACGTGGGTTGTGCTTG | |
| | | | | | GGAAAAGTTGGACCTGTTAATTAAAAGTAAAATAT | |
| | | | | | TTCCAAATCAATTTGGAAATGACTTGAAGTGTGAGG | |
| | | | | | GAAAGGGATTCATAAAATTTAGGTATAGGAGGCCC | |
| | | | | | TGGAAAAGGACATTTATCCTAGAGGGCACAGGGGG | |
| | | | | | TGTCTCTCTGGTAGGGGAAGGGTGGGGAGGTGGCT | |
| | | | | | TTATAAGAGTGGTCTGCCTTCTCCCTTTCTCACTTTT | |
| | | | | | CCTCACCCCTTTTCTCTCTTCCCCCGCAAAGCTGCTT | |
| | | | | | CCCTGCCCTGCCACCACCTTTAGTGCTTTGTCTTTTT | |
| | | | | | TCCCCTTTGCCCATGCTCAGCTGTTAACCCATAAAG | |
| | | | | | ACTTCGTTGATTTTGTGTGCATAGTGGATGGTATGG | |
| | | | | | CTGCATTAATCCCTTCACTGCCTGTATACCCTAGAA | |
| | | | | | TTTGTCCCTGACACTGACTTCAGAGCATGGTTTGAG | |
| | | | | | TTCATCTCCCATCATTCCCCATTGTTGTGCTTCCCGT | |
| | | | | | AAAAACTGCCAGCTTTATCATTTCCCCTGGCTCTGC | |
| | | | | | CCACACTGCATGTGTAGGGGCTGAACTATGGGCAA | |
| | | | | | GTGTCTGACCACCCAGCCAGGTGAGTGTGTGTCTTC | |
| | | | | | TAATGCAAGTCTGTTTCTGTTTTTGTTGTCTTTTTTAA | |
| | | | | | ACTCATAGAATTGATTGTTGAAAATAAGGCCATCA | |
| | | | | | ACTGCTAAAACAACTACTAAAATAATTCTTTTTAAT | |
| | | | | | ATAAAAATAACTTTGTCAAATTCACTTTCAGAAGAT | |
| | | | | | TTTTCAGATGTCCCTGTTGAGAGCATTGTTCTAGAT | |
| | | | | | AGGTTATATTTGAAACTGTGAGCAGAAGCATGTGA | |
| | | | | | GCCCATCTGCTATGATGAGTAATAGTCATTGAGGCC | |
| | | | | | TGAAACATACAGTGCTTTAAGCATGACTGTTATTAC | |
| | | | | | AAAGCATGCTTCTCCCACCCCACCCACCCCCTCAAA | |
| | | | | | GAAGGTAGCCATTGAAACATAAGGATGATAGATAG | |
| | | | | | AATGTATTACTTCAAATCTAACTCTTAGCTGGTGGA | |
| | | | | | GGATTTAGTAATTTAGTTGCTTTAGGTCTTGTAAAA | |
| | | | | | GCTCCTGCCGCTAACTTTAGGAGATGAGAAGTTTGA | |
| | | | | | CCCTTAATGTTCTTGATATTTTTTTAGATCAACTCCA | |
| | | | | | CAATTTACTGTGATCCAATCCATCTGCTTTCTATCTG | |
| | | | | | TTGTGCTCTATGATTGGTTCTCATTTACCTTCATTTC | |
| | | | | | TGTATTCTACTTTCCTTAAACTTTAAGGAAATCTAA | |
| | | | | | TCACAACTCCTGAAGACTTACCTTTCTTAGATCTGA | |
| | | | | | AACTTAAGATCAGTGTATTATAAAATGGAATCTCTT | |
| | | | | | AGCAGTCACAGCTACATAAATTGGGATTTTAATAGT | |
| | | | | | TGTCTGTGCTTTGAATTCTTTTCCTTTAAATGTCTGT | |
| | | | | | TTCTTTTATGTAAAGTTTTTCAGTTTGGGGAACGTGT | |
| | | | | | AGTCTTCCCCTCCCTTTTAATTTCTCACCAGGATCTA | |
| | | | | | AACCCCCCTTCTCTGTGAAGCTTAAATCTGCATTGT | |
| | | | | | ACTCTCCCTCCTCCCCCCCCATCAGTATCCAGCAGG | |
| | | | | | TTACCCTTCAGATAAAGAAGGGAAGAAGCCTAAAG | |
| | | | | | GACAGTCAAAGAAGCAGCCCAGTGGAACCACAAAA | |
| | | | | | AGGCCAATTTCAGATGATGACTGTCCAAGTGCCTCC | |
| | | | | | AAAGTGTACAAAGCATCAGATTCAGCAGAAGCAAT | |
| | | | | | TGAGGCTTTTCAACTAACTCCTCAACAGCAACATCT | |
| | | | | | CATCAGAGAAGATTGTCAAAACCAGAAGCTGTGGG | |
| | | | | | ATGAAGTGCTTTCACATCTTGTGGAAGGACCAAATT | |
| | | | | | TTCTGAAAAAATTGGAACAATCTTTTATGTGCGTTT | |
| | | | | | GCTGTCAGGAGCTAGTTTACCAGCCTGTGACAACTG | |
| | | | | | AGTGCTTCCACAATGTCTGTAAAGATTGCCTACAGC | |
| | | | | | GCTCCTTTAAGGCACAGGTTTTCTCCTGCCCTGCTT | |
| | | | | | GCCGGCATGATCTTGGCCAGAATTACATCATGATTC | |
| | | | | | CCAATGAGATTCTGCAGACTCTACTTGACTTTTTCT | |
| | | | | | TCCCTGGCTACAGCAAAGGACGATGATCTGCCTGCT | |
| | | | | | TTCACTGTGTGTTCATGGTGGCTTTTTGGACAATA | |
| | | | | | AAGAATCTAAAATGGGTGGGGAGGGTGGAAGAAAT | |
| | | | | | GGTGGACTGTATCTCTCACGTTCTGAAGCAGCTAAT | |
| | | | | | CCTCTTTCCCACATAGCCATCATCTTGTGTGTGTAGT | |
| | | | | | AAGAGGCCCATTTCTCAACTGTCTTTTAAATATCTA | |
| | | | | | AAGGTAGTTCCTGTAACAACTAGTTTTAATGAGTAA | |
| | | | | | AAAGTCAAAGCCTCAGCTCTAGTTGATATCCAAGTT | |
| | | | | | ATGATTTATTTTGCAACTACCTCAGGACAGAAAAGA | |
| | | | | | TTTATGGGGATTTTAAAAATCATTGAATAACTAGTT | |
| | | | | | AAATGAAATTTTAGCTACACACTGCCTCCCAAATAT | |
| | | | | | TAGTTGTGCCTGGTTCTTGTAATTTGATTTTACAGA | |
| | | | | | AAAGGAAATGACACTTGAGATCCTTGGAATGAACA | |
| | | | | | CAGCTTCTAAAGTGTGCATATACTTTTTTAACGTCT | |
| | | | | | CTTCTTCCATTACAATGTGTGTTTTGCAAGGACAGG | |
| | | | | | TTCATTTTTTTTAGCCCACTTTGTGAACTCCATTGTG | |
| | | | | | CTTTTTTCTGGTGTTTTATGCAAGTTGACTACTAATG | |
| | | | | | ACTAATGAGAACAATAATGAATGCATTGTTGCTGC | |
| | | | | | ATTAGTGTAATGTGGTGTGGTTTTGCACTTAAAATA | |
| | | | | | GGTATTCATATGCTCTAGTTGTAAATGTTCATGAAA | |
| | | | | | ATCCACTTCTCTACTAGTCGAACTGCTTTTAGTGTCT | |
| | | | | | CACCAGTGGTTTTACATCTGCAGAGTTTTGAGGGCT | |
| | | | | | GTGCTGACCTTTGAGAGGATTTGAAATTGCTTCATA | |
| | | | | | TTGTGATCCTAAATTTTATATTCACTATATTCCCTAA | |
| | | | | | AGTATACCTTAATAAATATTTTATGATCAGAAAAAC | |
| | | | | | AGCT | |
| 79 | 360380 | | 79 | 114 | GAGGTCGCGTAGGGCCTATTATGATGATTTCTACAG | MIKSSSLTRACPPHPRQQGGEQGNKITTKSLGVSHSPS |
| | | | | | GAGGTTGAAGAGATAAGACCCTTCCCTGTGCTCCCC | PGTLSETLQSPRNSLREAGRRPAIWTKLRYADADRAA |
| | | | | | CCCCCCCACTCCTTAATTACGGATTGAGCAGGGGAG | LRGEDPGGASSAGSSSQKTDDPERVAGTDCQAFGGGT |
| | | | | | GGGCCGGTGGGGCTCAGGTGAGCACACAGGGAGAA | GSGRLGSAFKMASPQGGQIAIAMRLRNQLQSVYKMD |
| | | | | | AGGGACGTGGGCGGGGCCTTACAGAGGGTGAGCGA | PLRNEVQGRQGYCCGRPAEEVRVKIKDLNEHIVCCLC |
| | | | | | ATCCGAAAAGACCTAGAACCTCGTTGCTGGGAGAC | AGYFVDATTITECLHTFCKSCIVKYLQTSKYCPMCNIK |
| | | | | | AAGTCCCGCCCTGCAATGATTAAATCATCATCATTA | IHETQPLLNLKLDRVMQDIVYKLVPGLQDSEEKRIREF |
| | | | | | ACCAGGGCCTGCCCCCCCCATCCCCGGCAGCAGGG | YQSRGLDRVTQPTGEGMSLAAGQ |
| | | | | | GGGAGAATGGGGGAATAAGATCACTACCAAGTCCC | |
| | | | | | TGGGGGTCTCTCACTCCCCATCCCCCGGCACCCTCT | |
| | | | | | CCGAGACTCTGCAAAGCCCAAGAAACTCCCTCCGT | |
| | | | | | GAAGCCGGGAGAAGACCCGCCATCTGGACGAAGCT | |
| | | | | | CCGCTACGCGGACGCCGACAGGGCGGCATTACGAG | |
| | | | | | GAGAGGACCCAGGAGGGGCTTCTTCAGCAGGGTCG | |
| | | | | | TCGTCACAGAAGACCGACGACCCTGAGCGGGTAGC | |
| | | | | | GGGCACAGACTGCCAGGCCTTTGGGGGCGGCACCG | |
| | | | | | GAAGTGGCCGGCTGGGATCAGCCTTTAAGATGGCG | |
| | | | | | TCTCCTCAGGGGGGCCAGATTGCGATCGCGATGAG | |
| | | | | | GCTTCGGAACCAGCTCCAGTCAGTGTACAAGATGG | |
| | | | | | ACCCGCTACGGAACGAGGTGCAAGGGCGGCAGGGT | |
| | | | | | TACTGCTGTGGTCGGCCAGCGGAGGAGGTTCGAGT | |
| | | | | | GAAGATCAAAGACTTGAATGAACACATTGTTTGCT | |
| | | | | | GCCTATGCGCCGGCTACTTCGTGGATGCCACCACCA | |
| | | | | | TCACAGAGTGTCTTCATACTTTCTGCAAGAGTTGTA | |
| | | | | | TTGTGAAGTACCTCCAAACTAGCAAGTACTGCCCCA | |
| | | | | | TGTGCAACATTAAGATCCACGAGACACAGCCACTG | |
| | | | | | CTCAACCTCAAACTGGACCGGGTCATGCAGGACAT | |
| | | | | | CGTGTATAAGCTGGTGCCTGGCTTGCAAGACAGTG | |
| | | | | | AAGAGAAACGGATTCGGGAATTCTACCAGTCCCGA | |
| | | | | | GGTTTGGACCGGGTCACCCAGCCCACTGGGGAAGG | |
| | | | | | TATGTCCTTGGCCGCGGGACAGTAAAGACCCCAGA | |
| | | | | | GCATTCTTCTTGCCCAGTTTTGCTCTCTGGGGAAAG | |
| | | | | | AGGAGTATGGAATGTGTGCCACCAGCCACCTCACT | |
| | | | | | ACCCTATCTTTCTCAGAGCCAGCACTGAGCAACCTC | |
| | | | | | GGCCTCCCCTTCAGCAGCTTTGACCACTCTAAAGCC | |
| | | | | | CACTACTATCGCTATGATGAGCAGTTGAACCTGTGC | |
| | | | | | CTGGAGCGGCTGAGGTGAGGAGAAGGTCAGGGGTT | |
| | | | | | GCAGGAGGTGACAGTGCCAATGACCCAGAGCCAGG | |
| | | | | | GAGGGTCTAGGGGAGAGGCTGAGCAGTGAGTGAGT | |
| | | | | | GCCTATCCCCTTGAAGAGAGTATATCATGGCTCTGG | |
| | | | | | GTGGGGAAGAGGAGGAAAGATAGGATTCCCTAACC | |
| | | | | | TGTGTCTATTTCCCCCCAGTTCTGGCAAAGACAAGA | |
| | | | | | ATAAAAGCGTCCTGCAGGTGAGAAGGGCTGAGGGG | |
| | | | | | AGGGCCTCTCTAAGGAGACTCACCTCCCATGGTCCT | |
| | | | | | TCCCTCACACACCTTGCCCTCTTCCCTCCCCTCCCTG | |
| | | | | | CTCCCAGAACAAGTATGTCCGATGTTCTGTTAGAGC | |
| | | | | | TGAGGTACGCCATCTCCGGAGGGTCCTGTGTCACCG | |
| | | | | | CTTGATGCTAAACCCTCAGCATGTGCAGCTCCTTTT | |
| | | | | | TGACAATGAAGTTCTCCCTGATCACATGACAATGAA | |
| | | | | | GCAGATATGGCTCTCCCGCTGGTTCGGCAAGGACTC | |
| | | | | | ACATCCAAAGGCGACAGCACCAGGATTTGCTCCCG | |
| | | | | | CCTTTGGCACAGAGGAGGACGGGTCCCTCTCTCAGC | |
| | | | | | CTGGCCAGTCTTTCCCAGGGCTTGATGGGAAAAAG | |
| | | | | | GACTTCCCTAGAAGGGGTTATTCCGAGGGTCCTCCA | |
| | | | | | ACCCTGCTACACATTCACAGAATTCAGTGGAATGTC | |
| | | | | | CGGGCCGGCAATCCGAGACTAAAGGTCGTTTATTG | |
| | | | | | ATAAGCCAGGCCACCCTCCCTGGGATCACACCCCCT | |
| | | | | | TCAGACTCCCCCCAACCATCCTACAGTCCTCAGGGG | |
| | | | | | AAGGGTGGGCTGAGGGGCCCTTTGAATAATATAAG | |
| | | | | | AACATTCCCCACTGACTACTACTTCCTCATTCTCTCC | |
| | | | | | TTAGCCATCCCCTTTGCTTTTACAATACAGTGTGAA | |
| | | | | | AGAGAAGAGGAGGTAGGGGCCAAGCCCCCACCCCA | |
| | | | | | TCCCACTCCCCTTCCCTCCCCAGATATTTATGTGAA | |
| | | | | | ATGAACTGCAGCTTTATTTTTTG | |
| 80 | 246666 | | 80 | 115 | CCTCCTTGCTTTCAGGACTCAGTTTCCTGGGTCCCC | MVGGGVGGGLLENANPLIYQRSGERPVTAGEEDEQ |
| | | | | | TTCACGGCCCCTCATCTCCTTACAGTCCAGGGTCTG | VPDSIDAREIFDLIRSINDPEHPLTLEELNVVEQVRVQV |
| | | | | | AGGGTCTCCGCGGTCCCCTCCCTACTCAGTCACGCC | SHFRGERVVPESQKGFCAAGAGVLYAHEHRRVSDPES |
| | | | | | ATTCTTTTGAAACGTACACGTGACCGCGGCACTTCT | TVAVAFTPTIPHCSMATLIGLSIKVKLLRSLPQRFKMD |
| | | | | | TAAGGAGCGCCCCCCTTTTCCTCGGTGGCTTTCAGT | VHITPGTHASEHAVNKQLADKERVAAALENTHLLEV |
| | | | | | TTCCTCACCTCCCGCGGAGACCACGGCCATGGTCAT | VNQCLSARS |
| | | | | | TTATCCACTTGACAAACATTTCACGAGCCCCTGCCG | |
| | | | | | GTCCAAGCTGTGGGGACGCCGTACTCCCGGGCCTAT | |
| | | | | | GGTGCAGCAGGGGAGGCAGGCGCGTCACCGGGAG | |
| | | | | | GTCCCGAGACACTAGGATCCCTGCCAGGCCAGAGG | |
| | | | | | CGACCAACCGTCCTGGATACGGGAGCTCCCGGCCA | |
| | | | | | GCCTGACTTCCAGGAGGAAGCGGTGTGGGGATTAC | |
| | | | | | CTCCGACCGCCTTTAGTGCCCCCTGAGACCTGGTTC | |
| | | | | | TGGCCTCTACGTTTCAGCCCGCTACTGGCTCGCACG | |
| | | | | | ACCCAGCGCCGCCGTGGTCCCTCTTCAGCGCCTTCT | |
| | | | | | GCTCCAGCGACCATCATGTTCCCGGGTCCGAGCAGC | |
| | | | | | CAGGGCCGCGGTCACCGCTTCTCTCGCACCTCAGGC | |
| | | | | | CGAGAACCCACAACGCGGCGTGTCCCTCGCGCGAC | |
| | | | | | TCCGTCGCCACGCCACGCCCCCTTCCCGTTCTCCGG | |
| | | | | | AAGTGCGCGGGTTGGAGCGGAAGCGCACGAGCAAA | |
| | | | | | ATGTTAGTTTCTCATTGTGAGTGATTCAAGAAAACA | |
| | | | | | ACGGTAACAGCCCTGCTAGGATCAGCGGTGGTGGT | |
| | | | | | TCCGCGATGGTAGGCGGCGGCGGGGTCGGCGGCGG | |
| | | | | | CCTCCTGGAGAATGCCAACCCCCTCATCTACCAGCG | |
| | | | | | CTCTGGGGAGCGGCCTGTGACGGCAGGCGAGGAGG | |
| | | | | | ACGAGCAGGTTCCCGACAGCATCGACGCACGCGAG | |
| | | | | | ATCTTCGATCTGATTCGCTCCATCAATGACCCGGAG | |
| | | | | | CATCCACTGACGCTAGAGGAGTTGAACGTAGTAGA | |
| | | | | | GCAGGTGCGGGTTCAGGTGAGTCACTTCCGAGGGG | |
| | | | | | AGCGAGTTGTTCCAGAGAGTCAGAAAGGTTTCTGT | |
| | | | | | GCAGCAGGAGCTGGCGTGCTCTATGCTCACGAACA | |
| | | | | | CCGAAGGGTTAGCGACCCCGAGAGTACAGTGGCTG | |
| | | | | | TGGCTTTCACACCAACCATTCCGCACTGCAGCATGG | |
| | | | | | CCACCCTTATTGGTCTGTCCATCAAGGTCAAGCTTC | |
| | | | | | TGCGCTCCCTTCCTCAGCGTTTCAAGATGGACGTGC | |
| | | | | | ACATTACTCCGGGGACCCATGCCTCACAGCATGCA | |
| | | | | | GTGAACAAGCAACTTGCAGATAAGGAGCGGGTGGC | |
| | | | | | AGCTGCCCTGGAGAACACCCACCTCTTGGAGGTTGT | |
| | | | | | GAATCAGTGCCTGTCAGCCCGCTCCTGAGCCTGGCC | |
| | | | | | TTTGACCCCTCAGCCTGCATACTGGTATCCTGGTCC | |
| | | | | | CAGCTCCTGCCAGGGCTGTTACCGTTGTTTTCTTGA | |
| | | | | | ATCACTCACAATGAGAAACTAACATTTTGCTTTTTG | |
| | | | | | TAATAAAGTTAATTTATATTCAGT | |
| 81 | 204305 | | 81 | 116 | CGGGAGCGCGCACGCTCGCGCACCCGGATCCCGG | MEAFQELRKPSARLECDHCSFRGTDYENVQIHMGTIH |
| | | | | | CTCCTGCATCCAGTCGCCATTCGGGAGGCCGCTGCG | PEFCDEMDAGGLGKMIFYQKSAKLFHCHKCFFTSKM |
| | | | | | CTGCAGGGCCTCGCGGAGCCGCCCGCGACCGCGAG | YSNVYYHITSKHASPDKWNDKPKNQLNKETDPVKSP |
| | | | | | CCGGGCCCTCCGCGCGGTCCATCGCCCACTGGACGC | PLPEHQKIPCNSAEPKSIPALSMETQKLGSVLSPESPKP |
| | | | | | CGCCCGCGGCCGGACCGGTTCAACTTCTCATCTTTG | TPLTPLEPQKPGSVVSPELQTPLPSPEPSKPASVSSPEPP |
| | | | | | TTCTTCTTCATATACTATAGGCTGTTTGCTGTGGTTT | KSVPVCESQKLAPVPSPEPQKPAPVSPESVKATLSNPK |
| | | | | | AGTCAAAAAGCCATGTAGAATGCCTGCCTTTTGAA | PQKQSHFPETLGPPSASSPESPVLAASPEPWGPSPAASP |
| | | | | | GACCACTTTTAAGGTGTCTAGTAAGACAGCAGCAG | ESRKSARTTSPEPRKPSPSESPEPWKPFPAVSPEPRRPA |
| | | | | | TATTGAAAGTTTTTAAAGAATATAACCGTGTGTGTT | PAVSPGSWKPGPPGSPRPWKSNPSASSGWKPAKPAP |
| | | | | | GGTAACAGACAGAAGAATGGAAGCATTCCAGGAAC | SVSPGPWKPTPSVSPGPWKPTPSVSSASWKSSSVSPSS |
| | | | | | TTCGTAAACCATCAGCACGTTTGGAGTGTGACCATT | WKSPPASPESWKSGPPELRKTAPTLSPEHWKAVPPVS |
| | | | | | GCAGTTTCAGAGGCACAGACTATGAAAATGTACAA | PELRKPGPPLSPEIRSPAGSPELRKPSGSPDLWKLSPDQ |
| | | | | | ATCCATATGGGTACCATCCATCCAGAATTTTGTGAT | RKTSPASLDFPESQKSSRGGSPDLWKSSFFIEPQKPVFP |
| | | | | | GAAATGGATGCTGGTGGGCTAGGCAAAATGATATT | ETRKPGPSGPSESPKAASDIWKPVLSIDTEPRKPALFPE |
| | | | | | TTACCAGAAAAGTGCAAAGTTATTTCACTGCCATAA | PAKTAPPASPEARKRALFPEPRKHALFPELPKSALFSES |
| | | | | | ATGCTTCTTCACCAGCAAGATGTACTCTAATGTATA | QKAVELGDELQIDAIDDQKCDILVQEELLASPKKLLED |
| | | | | | CTATCACATCACATCCAAACATGCATCCCCAGACAA | TLFPSSKKLKKDNQESSDAELSSSEYIKTDLDAMDIKG |
| | | | | | ATGGAATGATAAACCAAAAAATCAGTTGAACAAAG | QESSSDQEQVDVESIDFSKENKMDMTSPEQSRNVLQF |
| | | | | | AAACAGATCCTGTGAAAAGCCCTCCTCTTCCTGAAC | TEEKEAFISEEEIAKYMKRGKGKYYCKICCRAMKKG |
| | | | | | ACCAGAAAATACCCTGCAATTCAGCAGAACCAAAA | AVLHHLVNKHNVHSPYKCTICGKAFLLESLLKNHVA |
| | | | | | TCCATACCTGCCCTTTCAATGGAAACACAGAAACTT | AHGQSLLKCPRCNFESNFPRGFKKHLTHCQSRHNEEA |
| | | | | | GGTTCAGTTTTGTCTCCAGAATCGCCAAAACCTACT | NKKLMEALEPPLEEQQI |
| | | | | | CCTCTTACTCCCCTGGAGCCTCAGAAACCTGGCTCT | |
| | | | | | GTTGTTTCTCCTGAGCTACAGACACCTCTTCCTTCTC | |
| | | | | | CTGAGCCTTCAAAACCTGCCTCTGTTTCTTCTCCTGA | |
| | | | | | ACCTCCAAAATCAGTCCCTGTTTGTGAGTCTCAGAA | |
| | | | | | ACTTGCCCCTGTTCCTTCTCCAGAACCACAGAAACC | |
| | | | | | TGCCCCTGTATCTCCTGAGTCAGTAAAGGCTACTCT | |
| | | | | | TAGAATCCCAAACCCCAGAAGCAGTCTCATTTCCC | |
| | | | | | GGAAACATTGGGGCCACCTTCAGCCTCATCTCCAGA | |
| | | | | | GTCACCAGTTCTAGCTGCTTCCCCAGAACCTTGGGG | |
| | | | | | ACCATCCCCAGCTGCATCTCCAGAATCTCGGAAGTC | |
| | | | | | AGCCCGGACTACCTCCCCTGAGCCAAGGAAGCCAT | |
| | | | | | CCCCTTCAGAGTCTCCTGAACCTTGGAAGCCGTTCC | |
| | | | | | CTGCTGTCTCCCCAGAGCCTAGGAGACCAGCCCCCG | |
| | | | | | CTGTGTCACCAGGCTCTTGGAAACCAGGGCCACCTG | |
| | | | | | GGTCCCCTAGGCCTTGGAAATCCAATCCTTCAGCAT | |
| | | | | | CATCAGGACCTTGGAAGCCAGCTAAACCTGCTCCAT | |
| | | | | | CTGTGTCTCCTGGACCTTGGAAACCAATTCCTTCTG | |
| | | | | | TATCTCCTGGACCTTGGAAACCAACTCCATCTGTGT | |
| | | | | | CTTCTGCATCCTGGAAATCTTCATCAGTCTCACCCA | |
| | | | | | GCTCCTGGAAGTCTCCCCCTGCATCTCCTGAGTCAT | |
| | | | | | GGAAGTCTGGCCCACCAGAACTCCGAAAGACAGCT | |
| | | | | | CCCACGTTGTCTCCTGAACATTGGAAGGCAGTTCCC | |
| | | | | | CCAGTGTCTCCAGAGCTTCGCAAACCCGGCCCACCA | |
| | | | | | CTATCCCCAGAGATCCGTAGTCCAGCAGGATCTCCA | |
| | | | | | GAGCTCAGAAAACCCTCAGGGTCACCAGATCTTTG | |
| | | | | | GAAGCTTCTCCTGATCAGCGGAAAACTTCTCCTGC | |
| | | | | | TTCACTTGATTTCCCTGAGTCCCAGAAAAGTTCCCG | |
| | | | | | TGGTGGTTCTCCTGATCTCTGGAAGTCTTCCTTTTTT | |
| | | | | | ATTGAGCCTCAGAAACCTGTCTTCCCTGAGACCCGA | |
| | | | | | AAACCAGGTCCTTCTGGGCCATCTGAGTCCCCCAAA | |
| | | | | | GCAGCCTCAGATATCTGGAAGCCTGTTCTCTCTATC | |
| | | | | | GATACTGAGCCTAGAAAACCTGCCCTGTTTCCCGAG | |
| | | | | | CCTGCCAAAACAGCCCTCCTGCTTCTCCAGAAGCA | |
| | | | | | CGCAAACGTGCCCTTTTTCCAGAGCCCCGGAAGCAT | |
| | | | | | GCCCTTTTCCCTGAACTCCCCAAATCTGCTCTATTCT | |
| | | | | | CAGAATCACAGAAGGCTGTTGAGCTTGGTGATGAA | |
| | | | | | CTACAAATAGATGCCATAGATGATCAAAAATGTGA | |
| | | | | | TATTTTGGTTCAGGAAGAACTTCTAGCTTCACCTAA | |
| | | | | | GAAACTCTTAGAAGATACTTTATTTCCTTCCTCAAA | |
| | | | | | GAAGCTCAAGAAAGACAACCAAGAGAGCTCAGAC | |
| | | | | | GCTGAGCTTAGTAGTAGTGAGTACATAAAAACAGA | |
| | | | | | TTTGGATGCGATGGATATTAAGGGCCAGGAATCAA | |
| | | | | | GCAGTGATCAAGAGCAGGTTGATGTGGAATCCATT | |
| | | | | | GATTTTAGCAAAGAGAACAAAATGGACATGACTAG | |
| | | | | | TCCAGAGCAGTCTAGAAATGTGCTACAGTTTACTGA | |
| | | | | | AGAAAAAGAAGCTTTTATCTCTGAAGAGGAGATTG | |
| | | | | | CAAAATACATGAAGCGTGGAAAAGGAAAGTATTAT | |
| | | | | | TGCAAAATTTGTTGCTGTCGTGCTATGAAAAAAGGT | |
| | | | | | GCTGTTTTGCATCATTTGGTTAATAAGCATAATGTT | |
| | | | | | CATAGCCCTTACAAATGCACAATCTGTGGAAAGGC | |
| | | | | | TTTTCTTTTGGAATCTCTCCTTAAAAATCATGTAGCA | |
| | | | | | GCCCATGGGCAAAGTTTACTTAAATGTCCACGTTGT | |
| | | | | | AATTTTGAATCAAATTTCCCAAGAGGTTTTAAGAAA | |
| | | | | | CATTTAACTCATTGTCAAAGCCGGCATAATGAAGA | |
| | | | | | GGCAAATAAAAAGCTAATGGAAGCTCTTGAACCGC | |
| | | | | | CACTGGAGGAGCAGCAAATTTGATAACACAGTGTG | |
| | | | | | AATATTTGTTCTACAAAGGTGTTTGTTGGAACCATT | |
| | | | | | CTTTGTAAGTATAGCTTATCAGATAGCATAGTTGGA | |
| | | | | | TCAGTAGATGACATGTATGGTGTACCGTGTTTCACT | |
| | | | | | GTCTCAGTTGTGTTACTAAGAATGAGCATTTGATCA | |
| | | | | | TTTTTTTCTGGTCTCTGTCTATGTGACTATCTTGTAA | |
| | | | | | GTCAATAAATTTCTGTATAGTCCAGATGGATTAAAC | |
| | | | | | TTCTCATTTCTTTTAAATATGTATGAATAATAATAC | |
| | | | | | AAGGAAGTAGGCATTCCATTTAATAATCAAGAGCA | |
| | | | | | AGTTGTACTCAAAGCATTCAGTTAAAGTGTATCTGT | |
| | | | | | GTGTGGAACTAATTTCAGACAATAGAAAATATTAG | |
| | | | | | TTGAAATGTTTAAGAATTAGGCATGAAAAATAAAT | |
| | | | | | TTGAGAAATTTTGTTTCCTTACATGTATTTTTAAATC | |
| | | | | | ATAAGAGTTATTTTCTATCTGATGTAAAATTAGTTT | |
| | | | | | ATAAATCTTAATCAGCTTCTAGATGTTTATTAGCTTT | |
| | | | | | TATGTCATGAAATGTTGGAGTCTCAGGGTTGCTGAT | |
| | | | | | TTTCTGCTAATGGGAAAAATTGACTAAGTCTTTAAA | |
| | | | | | ATAGTTTGCAGCCTTCTCCCACAGGAGACAAGTGA | |
| | | | | | AAGATAAGTGTGATTTTAGATCTTTCTTGTCCATAG | |
| | | | | | TTGTTTTCAGTGGAGTCTTCCATTCTGTATCTTACCC | |
| | | | | | TAAGATCTGGTTCTTCCCTCCCCATCCCCACCCCCC | |
| | | | | | AACCCACCGCCTGCCAGCTCACACTAATAGATGATT | |
| | | | | | CTTAATTGCCAAATGTGTTAGAGTTTGTATATCCTA | |
| | | | | | CTCCTGGGCCTTACATGTCGCCTGTTGGGGCTTAAG | |
| | | | | | ACCAGGTTGATAAGTAGGAACTGAAAGTCTTCCAG | |
| | | | | | ATTCACAGTAGAAAATTTTATAGACATTTCTGTTAA | |
| | | | | | AGAAATATATCGATTTTATGTTTTTCAATTATGTTAC | |
| | | | | | TGTAAATACCTTGTACCTGTTCATGGATTATTTTATT | |
| | | | | | CTAAAATATTTTGTCAAATGTGTATCAACCAAATTA | |
| | | | | | AAAAGAAAGGTTTTCATGTCA | |
| 82 | 899425 | | 82 | 117 | CCGATCTCGGCCTCAGCGTGAGCATGCGCAGGTCCC | MPGMVLFGPALAIASDDLVFPGFFELVVRVLWWIGIL |
| | | | | | CGCCCTCGCTGCGTTTGCCTTGAGCGCGATAATTTG | TLYLMHRGKLDCAGGALLSSYLIVLMILLAVVICTVS |
| | | | | | GTGGCGGGGTCCGGCGGGTGCTGGTTTGTTCTCGGT | AIMCVSMRGTICNPGPRKSMSKLLYIRLALFFPEMVW |
| | | | | | GAACGGCGCGCGGGGTCTCTCCTGAGTGCGAGCTA | ASLGAAWVADGVQCDRTVVNGILATVVVSWIIIAATV |
| | | | | | CGGGACCTTCGCCATGCCGGGGATGGTACTCTTCGG | VSIIIVFDPLGGKMAPYSSAGPSHLDSHDSSQLLNGLK |
| | | | | | GCCGGCGCTGGCCATCGCCAGCGACGACTTGGTCTT | TAATSVWETRIKLLCCCIGKDDHTRVAFSSTAELFSTY |
| | | | | | CCCAGGGTTCTTCGAGCTGGTCGTGCGAGTGCTGTG | FSDTDLVPSDIAAGLALLHQQQDNIRNNQEPAQVVCH |
| | | | | | GTGGATTGGCATTCTGACGTTGTATCTCATGCACAG | APGSSQEADLDAELENCHHYMQFAAAAYGWPLYIYR |
| | | | | | AGGAAAGCTGGACTGTGCTGGTGGAGCCTTGCTCA | NPLTGLCRIGGDCRRSRTTDYDLVGGDQLNCHFGSIL |
| | | | | | GCAGTTACTTGATCGTCCTCATGATTCTCCTGGCAG | HTTGLQYRDFIHVSFHDKVYELPFLVALDHRKESVVV |
| | | | | | TTGTCATATGTACTGTGTCAGCCATCATGTGTGTCA | AVRGTMSLQDVLTDLSAESEVLDVECEVQDRLAHKG |
| | | | | | GCATGAGAGGAACGATTTGTAACCCTGGACCGCGG | ISQAARYVYQRLINDGILSQAFSIAPEYRLVIVGHSLGG |
| | | | | | AAGTCTATGTCTAAGCTGCTTTTACATCCGCCTGGCG | GAAALLATMLRAAYPQVRCYAFSPPRGLWSKALQEY |
| | | | | | CTGTTTTTTCCAGAGATGGTCTGGGCCTCTCTGGGG | SQSFIVSLVLGKDVIPRLSVTNLEDLKRRILRVVAHCN |
| | | | | | GCTGCCTGGGTGGCAGATGGTGTTCAGTGCGACAG | KPKYKILLHGLWYELFGGNPNNLPTELDGGDQEVLTQ |
| | | | | | GACAGTTGTAAACGGCATCATCGCAACCGTCGTGG | PLLGEQSLLTRWSPAYSFSSDSPLDSSPKYPPLYPPGRII |
| | | | | | TCAGTTGGATCATCATCGCTGCCACAGTGGTTTCCA | HLQEEGASGRFGCCSAAHYSAKWSHEAEFSKILIGPK |
| | | | | | TTATCATTGTCTTTGACCCTCTTGGGGGGAAAATGG | MLTDHMPDILMRALDSVVSDRAACVSCPAQGVSSVD |
| | | | | | CTCCATATTCCTCTGCCGGCCCCAGCCACCTGGATA | VA |
| | | | | | GTCATGATTCAAGCCAGTTACTTAATGGCCTCAAGA | |
| | | | | | CAGCAGCTACAAGCGTGTGGGAAACCAGAATCAAG | |
| | | | | | CTCTTGTGCTGTTGCATTGGGAAAGACGACCATACT | |
| | | | | | CGGGTTGCTTTTTCGAGTACGGCAGAGCTTTTCTCA | |
| | | | | | ACCTACTTTTCAGACACAGATCTGGTGCCCAGCGAC | |
| | | | | | ATTGCGGCGGGCCTCGCCCTGCTTCATCAGCAACAG | |
| | | | | | GACAATATCAGGAACAACCAAGAGCCTGCCCAGGT | |
| | | | | | GGTCTGCCATGCCCCAGGGAGCTCCCAGGAAGCTG | |
| | | | | | ATCTGGATGCAGAATTAGAAAACTGCCATCATTAC | |
| | | | | | ATGCAGTTTGCAGCAGCGGCCTATGGGTGGCCCCTC | |
| | | | | | TACATCTACAGAAACCCCCTCACGGGGCTGTGCAG | |
| | | | | | GATTGGTGGTGACTGCTGCAGAAGCAGAACCACAG | |
| | | | | | ACTATGACTTGGTCGGAGGCGATCAGCTCAACTGTC | |
| | | | | | ACTTCGGCTCCATCCTGCACACCACAGGGCTGCAGT | |
| | | | | | ACAGGGACTTCATCCACGTCAGCTTCCATGACAAG | |
| | | | | | GTTTACGAGCTGCCGTTTTTAGTGGCTCTGGATCAC | |
| | | | | | AGGAAAGAGTCTGTTGTGGTCGCTGTGAGGGGGAC | |
| | | | | | CATGTCTCTGCAGGATGTCCTTACGGACCTGTCAGC | |
| | | | | | GGAGAGTGAGGTGCTGGACGTGGAGTGTGAGGTGC | |
| | | | | | AGGACCGCCTGGCACACAAGGGTATTTCTCAAGCT | |
| | | | | | GCCAGATACGTTTACCAACGACTCATCAACGACGG | |
| | | | | | GATTTTGAGCCAAGCCTTCAGCATTGCTCCTGAGTA | |
| | | | | | CCGGCTGGTCATAGTGGGCCACAGCCTCGGGGGCG | |
| | | | | | GGGCGGCCGCCCTGCTGGCCACCATGCTCAGAGCC | |
| | | | | | GCCTACCCGCAGGTCAGGTGCTACGCCTTCTCCCCA | |
| | | | | | CCCCGGGGGCTGTGGAGCAAAGCTCTGCAGGAATA | |
| | | | | | TTCTCAGAGCTTCATCGTGTCACTCGTCCTGGGGAA | |
| | | | | | GGATGTGATTCCCAGGCTCAGTGTGACCAACTTGGA | |
| | | | | | AGATCTGAAGAGAAGAATCTTGCGAGTGGTCGCGC | |
| | | | | | ACTGCAATAAACCCAAGTACAAGATCTTGCTGCAC | |
| | | | | | GGTTTGTGGTACGAACTGTTTGGAGGAAACCCCAA | |
| | | | | | CAACTTGCCCACGGAGCTGGACGGGGGCGACCAGG | |
| | | | | | AAGTCCTGACACAGCCTCTTCTGGGGGAGCAGAGC | |
| | | | | | CTACTGACGCGCTGGTCCCCGGCCTACAGCTTCTCC | |
| | | | | | AGCGACTCCCCACTGGACTCTTCTCCCAAGTACCCC | |
| | | | | | CCTCTCTACCCTCCCGGCAGGATCATCCACCTGCAG | |
| | | | | | GAGGAGGGCGCCTCGGGGCGGTTTGGCTGCTGCTC | |
| | | | | | TGCTGCTCACTATAGCGCCAAGTGGTCACACGAAG | |
| | | | | | CGGAATTCAGCAAAATACTCATAGGTCCGAAGATG | |
| | | | | | CTCACCGACCACATGCCAGACATCCTGATGCGGGC | |
| | | | | | CTTGGACAGCGTGGTCTCCGACAGAGCGGCCTGCG | |
| | | | | | TCTCCTGTCCAGCACAAGGGGTCTCCAGTGTGGACG | |
| | | | | | TGGCCTGACCAGGGCCACTGGAAACTGTCCCAGGA | |
| | | | | | ACGATGGACTCACGCTTTTGTCCTTAAACTGACTTA | |
| | | | | | CCATCCGAGGAGTTCCCATGACGCCAAAACAGCGA | |
| | | | | | ATGTCCATCAACAGGAATCGGATGGGAACAGAATT | |
| | | | | | CCATGGTCTCAATGACTTAAGTTTATGGGAAGTCAT | |
| | | | | | TGTGGCCATAATGGTAGCAGAAGTAGTGAGCACGC | |
| | | | | | TCAGGTGATAGGACGACTCCTGAGACCCAGCGACC | |
| | | | | | GTGGAGACAGCCTCGGGAAGCCCTGGCCCGTGGAT | |
| | | | | | GGATCCCTTGGCTGTCTGAGGACTGCTCCAGAAGTG | |
| | | | | | CGGGAATCCAGGGCCCACCCAGAAGACCGTGAACA | |
| | | | | | GTTCCTTAGCCTCCCACCCCCAAGGCAGCTCTTTTC | |
| | | | | | ATCCAACTCAGTTTACAGGCGTGGTTTGTTTTTCAA | |
| | | | | | ACTGGGCTTCCTGGATGTACAAATGGAACTGTGGTG | |
| | | | | | AGGGTGCGGGCTGGGGTTTTCTCCTGGGCGTCACCA | |
| | | | | | AGGGCAGCCCTGGGCTCTGGCTGGGGATGAAGACG | |
| | | | | | AAACCCGATCGGGAAAGTAAGTGGAGCCCCCGGCC | |
| | | | | | CCGCCGAGCCACAGCCCCCCAACTGCCTATTCCCAC | |
| | | | | | TGCCCAGTGTTTGTCCACATCAGGAGTTGCTGATT | |
| | | | | | GAATTCTTGCTACTCTTCTGGCTCTGGGGTCGGCCA | |
| | | | | | GTGGATTCAGGAGTTGAAACAATAAAGCGCGCGTC | |
| | | | | | ACCATAGTGCTTGTGTGTACAGC | |
| 83 | 283 | | 83 | | AGGAACAGACTTGTTTTTGACTTGTCTATCTTTTCTA | |
| | | | | | AGGTTTTTTTCATCAGATACAAGTTCTTCCAGTTATT | |
| | | | | | GACACAGTCACTCCTAAGACTTAGCTTAAGTGTTAA | |
| | | | | | TGGCTCAACAATCTCAGCCGATTAACACTAATCATA | |
| | | | | | ATAATATTTATTGAATGTGTGCTTTGTGCCAGCCAC | |
| | | | | | TTTGCTGAGCCCTTTTCATGTTCTTAACCCTCACTAA | |
| | | | | | CTCCAATAACCAGAGTATGATTTTGTTCAGTGAAAC | |
| | | | | | CTGAGATTGTTTCTAGAGTAATCAGATAGTATTGAG | |
| | | | | | TAGCAGTGTTATCCCCAATAGTAGAAGAGAGCCAA | |
| | | | | | GGCTTCAGAAAATTGAAGAACTCTCCCAAGGTCAT | |
| | | | | | AGAGTTGGTTAAGGAGAGGGCCTCTGTTGTATATCC | |
| | | | | | AGATGGTTGACTATGAACCCACATTCTTAATTAGAT | |
| | | | | | TAAGAGTAGTAGAACTCTTTCTCTGCCTAGCTCTTG | |
| | | | | | TTGATCAGTAGAAAATTCACTCAGGGCTGGGCGCG | |
| | | | | | GTGGCTCACGCCTGTAATCCCAACACTTTGGGAGGT | |
| | | | | | CGAGGCGGGCAGATCACCTGAGGTTGGGAGTTCGA | |
| | | | | | GACCAGCCTTACCAACATGGAGGAACCCTGTCTCTA | |
| | | | | | CTAAAAATACAAAATTAGCTGGGT | |
| 84 | 404 | | 84 | | CTCCGCCAGACAGAGGTGCTGGGGCTGTGCAGGAA | |
| | | | | | ACGAAGTGATTAGAAATCCCGGATAAACACACAAG | |
| | | | | | CAGGCGTTGTCATGGTGACTGGGAAAAACACACAA | |
| | | | | | GCTGGCGTTGTCATGGTAATGGAGTGTAGGACAGG | |
| | | | | | CCTGGAGCCCCTCGGTCTCTTGCTGGCGGCTGGCAC | |
| | | | | | AGAGACGGGCTGCCGTGGGCTCTGACCTTAATACC | |
| | | | | | GGGTCACAGTCGCTTCTAGGACCAAGAGGACAGAG | |
| | | | | | ACCCATCACCGTATGCAGGGGCCTGTTTCCAGGCA | |
| | | | | | GACTGCCCAGTGCCCAGCTGAGCCTCGGGTGCAGT | |
| | | | | | GCGACCCCCGCAGGGCATGTCCAGACCCCAGGACC | |
| | | | | | CCCTCTCAGGTCTAGAAGATCCAGTTGGGCAGTGTT | |
| | | | | | GGTACCACCAAGAGTAGACAGGACAGAGGATCAGA | |
| | | | | | GACAATCCCACCCAGCAGGACCCAAGGACTCAGGC | |
| | | | | | AGTGGCTTTTCAGGTGTGTGGGCCGAGGACTGGGG | |
| | | | | | AGTCGGTGAATTCTGGGGCCCCTGGGGTGGCCGTTC | |
| | | | | | AGGAACTGCAGCAGCTCCCCCCACCACAGATGCTC | |
| | | | | | GCTGCCTACTGAAGCGGCCACGTGTTTGAATGAAG | |
| | | | | | AGCAGTTAGAGGAACGCTTGCAAGAGAATGTGTTT | |
| | | | | | ATTACCTGAGGTTATGACAATACAGAACATACAAT | |
| | | | | | GTTTTCTGTGGAAAATGTGATACTACAGAGGAAAA | |
| | | | | | GGTCACTTTAATTAAATGGCAATTAGAAGTAACAG | |
| | | | | | CATTGCAAGGTGGGGTGCAGCAGCTCACGCTTATA | |
| | | | | | ATCCCAGCACTTTAGGAGGCTGAGGCGGGTGGGTC | |
| | | | | | ACTTGAGGTCAGGAGTTCAAGACCAGCCTGGGCAA | |
| | | | | | CATGGTGAAACCTCGTCTCTACTAAAAATATAGAA | |
| | | | | | ATTAGCCACGCGTGGTGGTGCGCGCCTGTAGTCCAA | |
| | | | | | GATACTCAGGAGGCTGAGGCAGGAGAACCTCTTGA | |
| | | | | | CCCTAAGAGGCAGAGGTTGCAGTGAGCCAAGATCG | |
| | | | | | TGTCACTGCACTCCAGCCTGGGCAACAGAGCAAGA | |
| | | | | | CTCTGTCTCAAAAAGGAAAAAAAGAA | |
| 85 | 75 | | 85 | | TTAAAAACCAGGGGCGGTGGCTCACCCCTGTAATTC | |
| | | | | | CAGCACTTTGGGAGGCCAAGGCGGGCAGATCATGA | |
| | | | | | GGTCAGGAGTTCAAGACCAGCCTGGCCAACATGGT | |
| | | | | | GAAACCTCATCTCTACTAAAAATACAAAAATTAGA | |
| | | | | | TGAGTATGGTGGTACGTGCCTGTAATCCCAGCTACT | |
| | | | | | TGGGAGGCTGAGGCAGGAGAATCGCTTGAACCCAG | |
| | | | | | GAGGCAGAGGTTGCAGTGAGCCAAGACAGTGCCGC | |
| | | | | | TGCACTCCAGCCTGGTGACAGAGCGAGACTCCATCT | |
| | | | | | C | |
| 86 | 110 | | 86 | | CTAATGAGGAGTGATGCTGAGCATCTTTTCATATGC | |
| | | | | | TTACTGGTCATTTGTATGTTGTCTTTGGAAAAATGT | |
| | | | | | CTATTCAAGTCCTTTGACTATTTTAAAAATTGGGTT | |
| | | | | | ATTAGAGTTATCGTTGTTGTTGACTTGTAGGAGTTT | |
| | | | | | CTTTCTATATTCTGGATATTAATCCCCTATCAGATAT | |
| | | | | | ATGATTGCAAATATCTTCTCTTATTCCATAAGGTT | |
| | | | | | ACTTTTTCACTTTGTTGATTGTGTTCTTTGATGTATA | |
| | | | | | GAAGTTTTTAGTTTTGAAATAGTCTAATTTATCTGTT | |
| | | | | | TTTACTTTTGTGGTCTGTGCTTTTGGTGTCATATCCA | |
| | | | | | AGAAATCCTTTGCCAAATCCAACGTTATAAGGTACTT | |
| | | | | | TTAAGGTATTTTAGTTGTCTTAGTCTATATTTCTGTA | |
| | | | | | CTCACCTTTCTTTATCCACTCATCAGTTGATGGGCAT | |
| | | | | | GTAGGTTGGTTCCATATCTTTGCAATTCTGAATTGT | |
| | | | | | GCTGTGATCAGGTGTCTTTTTAGTATAATGATTTAC | |
| | | | | | TCTCCTTTGGGTAGATACCCAGTAGTGGGATTGCTG | |
| | | | | | GATCGAATGGTTTTTATAATTTTCTATTTTACCACAG | |
| | | | | | TTTCTCTCTGCATTTTTCCTCTTTGACCACTAACCAT | |
| | | | | | GTGAAATTCTCATATTGACCTTTATAATGATCATGA | |
| | | | | | ACTCTTAGTATCATTGGGAAGGCCACATTTGCCACT | |
| | | | | | TATGATTGTAAACCTTATCCTCCATTTTTCCTGTTAT | |
| | | | | | TGTTGGTGCAAAAAGCACCTATTATACCAGGACTTT | |
| | | | | | AAAAATCAGTCTGATAAGTCTTTGATAAGTCTAATA | |
| | | | | | ATAATAACTGATAAGTCCATTGAATTTGCTTCTGAT | |
| | | | | | TACTTTTTCTTTAGTAGCTAAACATGTATGTGGATCT | |
| | | | | | ATTTCTGGAAATTTTAGGCTCCAGTTTTTGTTGTTGT | |
| | | | | | TGTTAATAAAATGCAATGGAATGTAATGATCATCAC | |
| | | | | | TTTTCATTATGCTTTAAAATCTGGTAAATGGAGGCT | |
| | | | | | AGAACACTCCTGTAAGGCAAGAATATTCTCTCTGTT | |
| | | | | | GGAACTCAAATACACAGAACTGGGTAAATCTCAAT | |
| | | | | | CTTAATCTTTGATTCAGGACACAACATGGCTCTCTT | |
| | | | | | TTACTTGCTTTCTTTAATTGTTTTTTAATAATGTGGT | |
| | | | | | AAGCATTTCTGAATCTCCTATCCAATACAAAAACTA | |
| | | | | | GGACAATACAGACAGTAACTCCTATGGTTACAATG | |
| | | | | | AACACTCCTCTCCACTTAAATTAATTATTTACACTG | |
| | | | | | ATGAAATTGAAATAGCAAAATTTTAATGACTAAAT | |
| | | | | | ACTGTCTTTGATTTTTTGTTCCAGGTCTGTCAATATT | |
| | | | | | AACTTCTTATAATTTTCTTCAATGGCTTTGGGGGTA | |
| | | | | | CAAATGGCTTTTGGTCATATAGATGAATTCTACAGT | |
| | | | | | AGTGAAGTCTGAGATTTTACTGCACCGGTCACCTGA | |
| | | | | | GTAGTGTACATTGTACCCAATATGTGGTTTTTTATA | |
| | | | | | CCTTGCCCCCCCTCTTACCCTCCCCACTTTGAGTCTCT | |
| | | | | | AGTGTCCATTATGTCACTCTGTATACCTTTTGGTACC | |
| | | | | | CATCAGTTAGCTCTCACTTATAAGTGAGAACCACCA | |
| | | | | | CTGTATTTGGTTTTCCATTCCTGAGTGGCTTCACTTA | |
| | | | | | GAATAATATCCTCCAGCTCCATCCAAAATTGCTGC | |
| 87 | 70 | | 87 | | ATGATACAAGATGGTCATCAAATACTTCCTATTGTA | |
| | | | | | GTTGAAGGTCAATTTCTCTTTCCACTTTTCCTCAAGA | |
| | | | | | TGCAGAATGGCTGGTTTTTTCTCCCCACGGTTTCAAA | |
| | | | | | ACTTCACTAGTTAGAGATAATACTACTGCTAAATTT | |
| | | | | | TAATCTATTATATTTGCATCACAACTTTTATTATATC | |
| | | | | | AAAGTAATTGATACAGCTATCTATACTTGTCTTTTG | |
| | | | | | TCTAGGATATGCTCTCCAAATCTTGATTCTCTTTAA | |
| | | | | | AAGATAATGGCATACTTCCTAACATATCCAACTTAA | |
| | | | | | CAGCATCAATTTTAAATGCTGGACCCTTTAAGTACT | |
| | | | | | TATGCATTATATTTATAAGAACATGTCTATGGCCGG | |
| | | | | | GCTCGGTGGCTCATGCCTATAATTCCAGCACTTTGG | |
| | | | | | GAGGCCAAGGTGGGTGGGTCACTTGAGTCCAGGAG | |
| | | | | | TGTCTACAAAAA | |
| 88 | 310 | | 88 | | GGGCCCTCATAATAAGCATTGTTACTATTGGAAGTT | |
| | | | | | GTTTTCACATTCTTTCCAATATTAAATATGTATTTTT | |
| | | | | | TTAAGTAATGATAATATTTTCCAGTGGCTCATTTGG | |
| | | | | | ATGAGAACTACCCTCTATTTTTAATATTAAAACTAC | |
| | | | | | ATCCAACTCATCATTTAGCCTTTGGTTGTACAGTTG | |
| | | | | | TGTAATGGGCTATGGACTGTTACACACCTTACCACC | |
| | | | | | TCTAGGCCTATGTTTTTTCTTTCCCCATATATTCTGA | |
| | | | | | TGGGGATAAATACTGTTTTTGCCTCTCCCATAGGAAT | |
| | | | | | GGAATACATTTATTCTAAAATGATCTTTCACAGAAG | |
| | | | | | TAAGAGAGAGGGAAACCTAAATATACCTCTAAATT | |
| | | | | | GTTTGAAGTTGGTCCCAGCAGCATAAAATGGGTTG | |
| | | | | | GCCCCAAAGGGTTGGAGGGTGGGCTTGGTTATCAG | |
| | | | | | TATTTGTTTTCAGAATGAGATGGGAGCATCTTTCCT | |
| | | | | | TTGCCACGTGCTTTGTGCTTGATAACATCATGCTTG | |
| | | | | | GTTCAAACGACAACTCAGCACAAAGCCTTGAGTAT | |
| | | | | | AAATTGTTGGAATCAAAACATCTCATTCTGATGACG | |
| | | | | | TGGTTTAATTTTTAATTTTTTTTTTTAATAGGGGTG | |
| | | | | | GGAGGGAGGGTACTTTGCCCCAGAAGGGAGGGTGT | |
| | | | | | CTGCACTAAGGATTAGAAACACTTTGGAAGCTCAT | |
| | | | | | AACCTCATCAGAAACTGCCTTTAGCCACACTCCTGA | |
| | | | | | CCTTCTAGATGAGTAACAAAAAAATGAAATAAGTT | |
| | | | | | CTTGGAAATTAAGCCATTTATTTTAATTTGCTATTTT | |
| | | | | | TTTCAATGTTCTAGGTATCTTTAAATTTGTTATTGTG | |
| | | | | | GAATCATTTTCCTGCCAGATACCTTTATCAAAATTA | |
| | | | | | TTGGCCTCATGAGAGCTGAAGTAAGTCAGCTTTTTG | |
| | | | | | GTGAACTTTAGTGGACTTCTGTGAGATTGTAGTTGT | |
| | | | | | ACTTTGTATCTCTAAATCTAAAGATAGTTTTTTAAA | |
| | | | | | ACTCCCAAAGAAAATCTGCTCTCCTTTCTGATCTAA | |
| | | | | | AAACTCATCTTTGGGGTAAAGAGTTAAGTGTCCAA | |
| | | | | | AGGTTGTCACAGTTCATGAGGTCAGAGGGAGCTAG | |
| | | | | | CCTGGCACCTGGACTCTGCCCATCCACAGCTGACAG | |
| | | | | | ATTCCAACAGAAGTGTATTTAAATTCTCCAGTAGAC | |
| | | | | | AATGCTGGGTAAGGGAGGGGGTAGGGCTGGGTTAT | |
| | | | | | TAAGATACAGGCTGCTGTATTTTACATTGGTTGTGG | |
| | | | | | GGGAAGGGGAGCCTGGAGAAAACAAAGTCACTATT | |
| | | | | | CCCTTTTTTGAAACAGGAAAAAAAATTATTTTTTGT | |
| | | | | | TCAGTAAAAATGGTAGAGAATTCCAATGTCCCTAG | |
| | | | | | CCACAAGGGACCAGTTCCACTGAGAAGTGAACAGT | |
| | | | | | GGGAACTCAAAATTTCAGAAACATTGGGGGAAGGG | |
| | | | | | AAAATTGGCTTTCTCTTAATTGGCAGATGTTCCAGT | |
| | | | | | GGGGGGGGGGGGGGCTCTGTTTTTGTTGGGATGTGT | |
| | | | | | TATGTTGTATGTACGCATATATGGACCGGAGTCTGC | |
| | | | | | TGAGTTTATAAGGTTCCAAAAATATGGTAAAATCTT | |
| | | | | | GGTTTTTGTTAATTTATCTCAATAAAAGCCCACTGG | |
| | | | | | AACTCCA | |
| 89 | 212 | | 89 | | AAAATTGTCAATGTGGATGATTCTTTAAACCATAAT | |
| | | | | | TTGGGCCAAAAGCTGAGCATCACACCAAGAAAATA | |
| | | | | | TCTCTGCTTCTAGACATCAAGAAAGAGAGGTGGAG | |
| | | | | | ATAAAGGAAAAAACTTAATCCCGAATTGATAGGAG | |
| | | | | | TGAGAGACAACAAACCTTAGGACAGGGAATTCTTA | |
| | | | | | ACTTGTGGCAGAGCAAACAGTAGAAACTCATGAGA | |
| | | | | | CGTGTTATCCAATAATAGAAAATAGGAACATGAGA | |
| | | | | | TTTATTCCACTAGACAGTACTAGGACTCTACATGTA | |
| | | | | | AACTCATGGGAATTGAAATAAAGTTCTCTGCTGTAA | |
| | | | | | TTGGAGCAAGATAGACTGAGGAGAGAGTAAACCAC | |
| | | | | | GAATGCTGGCTCAAGACAAAAAACCTAGCAGAGGT | |
| | | | | | GCATTGCAGACATACCCATGAAGGAAAAACTTACA | |
| | | | | | CAAGGTCACCCTAAAGGAAGGACATTGTTAAGCCC | |
| | | | | | TTTGAAATAATGGGGTGGAGAGGAAAATGAACTGA | |
| | | | | | AAAAATGAAAAACACCCACAGGAAGAAATCAAAG | |
| | | | | | ACGATTGTGTCAACCCCAGGGCTACAGAAGTGAGG | |
| | | | | | AATAAAATTGGCTATTTCCGGACACTGACTTTCTTG | |
| | | | | | ATTTGTTGAACATACGTGAAAGCAGGACATGCCAT | |
| | | | | | GGTCGCTGGTTGCATCAAATAGAAATGACTCATTGG | |
| | | | | | AATGTTACCTCCAAATCCTTACATGAAGAGTAAGCA | |
| | | | | | AAAGATGAAAGCTTTTATGATCCTTTAGAAAAGA | |
| | | | | | AT | |
| 90 | 67 | | 90 | | CTTGCGCACTCAGTCACCAGCCCCCTTCTGGGGTCC | |
| | | | | | AAGCTGTGTCCCCTTCTCTAAAGAGGTAAGCCCTGA | |
| | | | | | GTCATGGGAAGATGGAAACCGGGGTCATGAGACA | |
| | | | | | GGATGTTTTTTAAGCACCGTGGTGTCTTGTTGACTT | |
| | | | | | GCACATGCACGGGGGTCTTGGGTAACCACAGGGCT | |
| | | | | | CAGGGTATTTGCAGGAACAGTTCAAGTGCTCACTTG | |
| | | | | | TCTTGGGGCTGTTTATGGGGAAGTGGTTTCCACAGT | |
| | | | | | GAGAGGAGGTGAGATATTGTTTGTCACCCCGGACCA | |
| | | | | | CACTTAGCTACTTCCTTCTCACTAAAGCTCTGTAGT | |
| | | | | | CATATTTTCCCTGGCAGAGCAGAAACTTCTATGTTA | |
| | | | | | TCCCACAGCTGTTCTAACGGTGTAGACTTGACTTAT | |
| | | | | | GCAATGATGCCAGGAGTCCTGAGCAGCACAGCCCA | |
| | | | | | ACTTCAATCACACACAGATGGACAGAGCTGTATTA | |
| | | | | | GCAAAGCCTGAGCTACTGAGCGATGAGAGTACAGC | |
| | | | | | CAGGCTTTCAGACATCTG1TCATCAAGAGAGATAT | |
| | | | | | GCGCTAAGCCAAGGACCTAAAGATGTGTTTAATAT | |
| | | | | | GGGTGCTAATATGCATAAGGAACCTTGAAATAAAT | |
| | | | | | GTTCTTAGCCTTTGGCCAAGAGGGTCCATGTCTAGG | |
| | | | | | AATCTATTGTCCATAGAAATAAATTCAAATATGGAA | |
| | | | | | AAAATGAACAATGCATAAGTGTATTTGGTCCCCAG | |
| | | | | | CATATTTATAGCAACTTAAAATTGGACCCAATTTAA | |
| | | | | | ATGCCTATGATATGGAAATGGCTAAGAAAATTATG | |
| | | | | | GGATCTTCCCTTGATTGGCTATTAGGCAGCCTTTAC | |
| | | | | | AAACAATGCAGTGACATGAGAAATGCTTATGTTAT | |
| | | | | | GGTAAGCTTAAAAAACTCAAGATGCAAATCAGCTT | |
| | | | | | ATTTTAATCAGGAGCCACCTAGCATTTGGGATGTGG | |
| | | | | | TCAATCCCACATAATGTATTTTTGTGGGTGCAGTTC | |
| | | | | | CCAGGAAAGAGGAGGAATAAAAACGGCAAGTATG | |
| | | | | | AAGTGTCTCCTTCGCTTGCAGTCTCCTTGTCTACCCC | |
| | | | | | TTTGTCCATCCACTATGAAAGGACTCCCTTCTGTTC | |
| | | | | | CTTAATATGGACAATTTCTATTGAGGACTCATTGTT | |
| | | | | | CTAAGAATTGTCTCATCTCCTCCTGCATCCTCAGTG | |
| | | | | | CCCGATCTTTGGCTTCTATGAAGGAAGGTGGGTAGT | |
| | | | | | GCGTATGGCAGGTCCAGTTCTACCTTTCTTAGTATG | |
| | | | | | TTCTGGCGTGGGTATGTAGCCCCATTTTCTAGTGGT | |
| | | | | | TACCTTGACATCATGAAGAGTTTATGTCTCTTTTGC | |
| | | | | | CCTAGGTTTGGGCAATAGTCATTCACTGTGCAACAG | |
| | | | | | GAAATACACGAGTCAGCATCTTATTAAAAATAAAG | |
| | | | | | TCATTCAGGAAAGTGGACGACAGTTTCTAATCTAGA | |
| | | | | | GAGCATAGGAGAAGAAATGTTTACCACACACAAAG | |
| | | | | | TAAGTTGCCTTTTATATCACGAAGACAAAAATAAC | |
| | | | | | AGGAAAAAGACAAACACATTATAGTGAAAACTTGT | |
| | | | | | TTTTCCTAACCAGCATCTATTCTGCATGTTTCCTGAT | |
| | | | | | GCCCGAAACTCACATTTCCTCAGGAAAATCTCCCTT | |
| | | | | | CTGCACCATTCTCAGGCTTTAAGTTTATGTAAAATT | |
| | | | | | CAGTAAACCCAAAGATTCAAGTTATGTGCCTTGATT | |
| | | | | | AACTTAAGCAAATCAATGAAACCCATCCCCATAAC | |
| | | | | | CACAGCGACAGGTTAGGAAATTCGGTTCCTAAGTC | |
| | | | | | AGTCACATCCGAAAGGGCCTAGTGATGTTTTTTTCC | |
| | | | | | AGTGGGATCACAGACTCACTCTTCCTTGCAGAAAAT | |
| | | | | | GAACAAAGGATTCATGTAACACTGGCAGGTACTGG | |
| | | | | | CAGCCACCCAGGGCCTCTCACAGGAAAGGGAGATC | |
| | | | | | AGAAAGAGAAGCAAAGAGGACTCATGAGATACCAT | |
| | | | | | AGGGCTGCTGCGTCCAGCCTfGCCTGGAGCTAGGG | |
| | | | | | CCACCTCGATGCCCTATAGTCTTGGAGCCACAACGT | |
| | | | | | GCATTTACTCAAAGCCTCTTTGAGTTTGGTTTGCTTG | |
| | | | | | TTTGCTTTCTGCCTGGAAACTGCCAGCATCCTGAGA | |
| | | | | | GATACGAGATCTGCATCTGTGCAGAGACACAGGGT | |
| | | | | | TTGTTAAAAGTCACAGGCCCTGACTGAAGTGTGGA | |
| | | | | | ACTGGCTGAAATGAGAAAGTGGTAATTTGGGGAGG | |
| | | | | | ACCTTGTGAAATGGAAGGAGTTTTAAACCTTACATG | |
| | | | | | CATCAGAATTACCTGGAGCCTTGTGAAAACACAGG | |
| | | | | | TTGCTGGGCCCTAGTCCATTAAGAAAGGAAGTGGG | |
| | | | | | GCTTAGAATGTCATTTCTCCCATGTTCCCAGGTGA | |
| | | | | | TATTCACCATGCTGTCCTGTCTGGGCACTACCTTTTG | |
| | | | | | CCATACCCATTACAAGGTATTGCACGTGCTGGTTGA | |
| | | | | | ACTATGGTCTGTCTTATTTTGGTGCTAAAAGCCTGT | |
| | | | | | GCCAAATACCAACGCTGCAGCATTAAGGAATGTGA | |
| | | | | | TAGAAAAGATTCTGAATATAGGCCAGGCGCAGTGG | |
| | | | | | CTCACGCCTGTAATCCCAGCACTTTGGGAGGCCGAA | |
| | | | | | GCAGGCAGATCACGAGGTCAGGAGATCAAGACCAT | |
| | | | | | CCTGGCTAACATGGTGAAACCCCGTCTCTACTAAAA | |
| | | | | | ATACAAAAAATTAGCCGGGCGTAGTGGTGGGCACC | |
| | | | | | TGTAGTCCCAGCTACTTGGGAGGCTGAGGCAGGAG | |
| | | | | | AATGGCGTGAACCTGGGAGGCGGAACTTGCACTGG | |
| | | | | | GCTGAGATCGCGCTACTGCACTCCACTCCAGCCTGG | |
| | | | | | GCGACAGAGCAAGACTTCGTCTC | |
| 91 | 371 | | 91 | | AATGAATTCCAGAATCCGGGGCAGGTTGGTAGGTC | |
| | | | | | CCAATCCCAGGGGAATGTGGTAAAAGTGGTACCCG | |
| | | | | | GTTTTGGGATCGGAAGGGTCCAATAAAATCCTTATT | |
| | | | | | TAATAATTCGGTACCCGAAGGCCAGTGTAATCCCA | |
| | | | | | AAAAGGAATAAAAACCAATAGTTTTGGTGGCTTCC | |
| | | | | | GCCGGAATTTTAAAAAATGGTTTTTAAAATAAAAA | |
| | | | | | AGTTAAGGTCCCTTTTAGGTAATTATTTTTAAGACC | |
| | | | | | AATTGCCAAATATCCACCCGGTAAACCTAATAAAA | |
| | | | | | CCCCCCCCTTCTTAAATACCATTTAACCATTGGGCA | |
| | | | | | AAGGTCCATTAGGGTGATTTGGCCCGATTAAAATAT | |
| | | | | | TTTTAAAGACCTAAAAAAAATGCCTTCCGGTTTCCC | |
| | | | | | GGCCATTAGGCAGGAATTTTTAATGATTACCCCATA | |
| | | | | | AGCCTACCATTTTTTTTTTTACCCCCAAAAATAAAA | |
| | | | | | ATTGTGAA | |
| 92 | 262 | | 92 | | ATTACAGGCGTGAGCCACCAAGCCTGGCCTAAAAC | |
| | | | | | ATTTAAAAATGTTTATTTTAAACATACATAAGACAT | |
| | | | | | GCACACATAAAGATACGCATAGCATGATTGAGGGC | |
| | | | | | TTGGTGTTTTGTTTCTGTAACACTGGATTTGAAACG | |
| | | | | | AAACTATAATGAGAATGTATAGCAGGGCTGGGCGA | |
| | | | | | ATGACAGGCTTGCTTATGACTGGAGGGTCAAGGGC | |
| | | | | | TATTGAGTGCAAAAGCTGGATGTAATCAGATTAGCT | |
| | | | | | CAGTGTTTTGTTTTTATAGCTATGCATTTAGCGTTT | |
| | | | | | AAACCATGGTAAAGAACAGCTTTTAAAAAAAAATC | |
| | | | | | GCTTCTCAGCCTTTTGGCTAAGCTCAAGTGTAAAAA | |
| | | | | | AAAAAAAAACAGCTTTAAATCTCAAGCTTTTGCCCC | |
| | | | | | TAATCTTTAAAATTTCATTGAAATAATTATCAGTTT | |
| | | | | | ACTGTTTCACTGCACCACAAATTTAGTTTCAGGTGT | |
| | | | | | ATCTTGAAACTCATTGATATGCTAATAAGTTTTATT | |
| | | | | | AAAATTGTTAAATTCTTCCTATGAATATACTTTTTA | |
| | | | | | TACAGATGTGACTTAAGTATTTAAATGTTTTACTTA | |
| | | | | | TTCACAAAATAACAAAGAATGGC | |
| 92 | 262 | | 28 | | GATCGTGCCATTGCACTCCAGTCTAGGCCACAACAG | |
| | | | | | CAAAACTCCGTCTAAAAATAAATAAATAAATAAAA | |
| | | | | | CTGAATGAATATAAACAGAAACCACAGATGCTATT | |
| | | | | | ACATATTAAATTGATAATATAACCATTACAGGCGTG | |
| | | | | | AGCCACCAAGCCTGGCCTAAAACATTTAAAAATGT | |
| | | | | | TTATTTTAAACATACATAAGACATGCACACATAAAG | |
| | | | | | ATACGCATAGCATGATTGAGGGCTTGGTGTTTTGTT | |
| | | | | | TCTGTAACACTGGATTTGAAACGAAACTATAATGA | |
| | | | | | GAATGTATAGCAGGGCTGGGCGAATGACAGGCTTG | |
| | | | | | CTTATGACTGGAGGGTCAAGGGCTATTGAGTGCAA | |
| | | | | | AAGCTGGATGTAATCAGATTAGCTCAGTGTTTTGTT | |
| | | | | | TTTATAGCTATGCATTTTAGCGTTTAAACCATGGTA | |
| | | | | | AAGAACAGCTTTTAAAAAAAAATCGCTTCTCAGCCT | |
| | | | | | TTTGGCTAAGCTCAAGTGTAAAAAAAAAAAAAACA | |
| | | | | | GCTTTAAATCTCAAGCTTTTGCCCCTAATCTTTTAAA | |
| | | | | | ATTTCATTGAAATAATATCAGTTTACTGTTTCACTG | |
| | | | | | CACCACAAATTTAGTTTCAGGTGTATCTTGAAACTC | |
| | | | | | ATTGATATGCTAATAAGTTTTATTAAAATTGTTAAA | |
| | | | | | TTCCTTCCTATGAATATACTTTTTATACAGATGTGAC | |
| | | | | | TTAAGTATTTAAATGTTTTACTTATTCACAAAATAA | |
| | | | | | CAAAGAATGGCAAAAAAAAAAGCATAAGCTCAAGTG | |
| | | | | | TAAAAAAAAAAAAAAAGG | |
| 93 | 65 | | 93 | | AATTTTTTGTATTTTTAGTAGAAATGGAGTTTCACC | |
| | | | | | ATGTTGGCCAGGCTGGTCTCAAACTCCTGAGACCTC | |
| | | | | | CACCTGCCTCGACCTCCCAAAAAGCTGCAATATCAG | |
| | | | | | GCATGATCCATCGCACCCGGCCACCCATGTATTCTT | |
| | | | | | GATTGAAAACATTTGCTCATGTCTTAGTTCTACAGC | |
| | | | | | TGACCTTCTTTCACTGTTTTCAAGGTCAATAACTGT | |
| | | | | | GTGTTCACACTTCTGCATTTTATAAATGTTACTGTG | |
| | | | | | ATTTTCTTGTAATGAAGAATTAAATGTTGGGAGTCA | |
| | | | | | ATGGCATCAGAACCTTGCAAAAGAGGTTTTTTTAGC | |
| | | | | | CCAGGTATGTGGAAGACACTTCTTTAATTTTCAATA | |
| | | | | | ATGGGTGTGATAAAGACCAACCCTTCCCATTAGCCC | |
| | | | | | TTCCAGGCCCACATGTAAGAATTCAGACACATCTTT | |
| | | | | | TCACTCATCTCAGACCTTCTCAGGGTAACTCGGTGA | |
| | | | | | AAATGTCTTCACTCTGAGCCTCAGTGAGCCTCCCTG | |
| | | | | | CAACTTGCAGATGAGGGGCTAGACCGGAAAAGCTC | |
| | | | | | AACCTGAGTGACCCTGGCCCCTGAAATGATTGGCA | |
| | | | | | AAATAGAGTGGGTGTCTGGATGTGGCTTTTTTTCTG | |
| | | | | | TGAGAGGGGACTGTCCAGTTGTAATTAGAATTTTAA | |
| | | | | | ATGGGATGCAGTACCCTAAAAATGAAAAAAATAAA | |
| | | | | | AAGAAGAATGGAAGAAACAGAGTTGTAGACTCAGA | |
| | | | | | CACAGAGACCATCTTCGGGGCCTTTCTCTGTGTGAG | |
| | | | | | GACATCACAGCGAAATCTAAAGCAGGTCATGTCAG | |
| | | | | | TCCCTGGCAGGGAACCCTCCACCAGCTTCCCGTGTT | |
| | | | | | CCCCAGGACAAAAGCCCCACTCCTCACTGTGGCTCC | |
| | | | | | ACAGCCCTGTGTCCAGGGCCCCTGCCAGTGTCCAGC | |
| | | | | | TTCCTCCTGGGAATTGCCCTCATCTCATGACTACC | |
| | | | | | TCTGCCCCAGTCACAGTTGCTTTTCTCTTTTCCCAAA | |
| | | | | | CATCAAAACCCTTCCTGTCTCAGGTTATTGTCCCTG | |
| | | | | | CTCTTACACTATGTACCTAAATGATGACAGCACTGT | |
| | | | | | CCCTTTCTCCTCCTTCAGGTCTAGGCTCAGAGATGT | |
| | | | | | CTCCCATGCCCTCCCACCCCCATCTGAAGATYCCTC | |
| | | | | | TGCCTGTCAGTCTCTCACGTTTACTCAGG | |
| 94 | 325 | | 94 | | AGAAGTAAAATTATCTCAATTCACATTTCATTTATG | |
| | | | | | ACTTTATTGATAGAAAACCTTAATAATACACATACA | |
| | | | | | CAGGATTCTATAAGCCTTAATAAAGAAGTTCAGCA | |
| | | | | | AAGTAGCAGATACAAGCTCAATATGACAATCAGTT | |
| | | | | | TAATTTCTGTACAATGATCATGAACAATCTATAAAA | |
| | | | | | GAACAATTTCATTTATAATAACATAAGCAAGTGTGT | |
| | | | | | AAGTATATAGTTAACGAAGGAAGTGTAAGATATAA | |
| | | | | | AACATTGTGAGAAATTAAATAAGACCAACAAATGA | |
| | | | | | AAAGTCATCTCTTATTCATTGATTGGAAGATATAAT | |
| | | | | | GTTGTTAAGATAGCAAGCCACTAAACTGACCTACA | |
| | | | | | GATTCAATGCTATCCCTAATCAAAATTGCAACAGCC | |
| | | | | | TTTTTGGCCTACAAGCTGCTCTTGAAATGCATATAA | |
| | | | | | AAATACAAGGGACTGAATAGCCAAAACAGTTTCTA | |
| | | | | | AAATAAAAACAAAATTGTAGGACTCAGATGTCTGA | |
| | | | | | TTTCCAAACCTAATACAAAGCTG | |
| 95 | 29 | | 95 | | GGTATATTTTATGTGCTGAGAAGTGTCAATCTAGAA | |
| | | | | | TTCTGTAGCAAACAAAACTATCAGGAAAATGGGCC | |
| | | | | | AAAGACATTTTGGATAAAAAGAGTTTACTACCAAC | |
| | | | | | ATGTCCTCATTAAATGAACTTAGGAAAGTTTATTCC | |
| | | | | | AGGAATCAGAATTAAGATCAGAAAGAMCATGTAA | |
| | | | | | GACGTAAGAAGAGATGGTGAGCAAAGAAAGTGGT | |
| | | | | | AAATGAGGCCAGGCACAGTGGCTCACACCTGTAAT | |
| | | | | | TCCAGCACTTTGGGAGGCCAAGGCGGGCGGATCAA | |
| | | | | | CTGAGGTCAGGAGTTCGAGGCCAGCCTGGCCAAGA | |
| | | | | | TGGCGAAACCTCATCTCTACTAAAAGTACAAAATTT | |
| | | | | | AGCCAGGCGTAGTGGTGCTTGCTTGTAATCCTGGCT | |
| | | | | | ACTTGGGAGGCTAAAGCAGTAGAATCGCTTGAACC | |
| | | | | | CAGGAGGCAGAAGTTGCAGTGAGCTGAAATTGCGC | |
| | | | | | CACTGCACTCCAGAGCCTGGGCAACAAGAGCAAAA | |
| | | | | | CTCCGTCTC | |
| 96 | 103 | | 96 | | GTGCTGGTTCAGGGGGAAGGAGGAGCACAAAGTGC | |
| | | | | | AAAGGGCTTTCTACCAGTGTCCAGTGTGTTATGAG | |
| | | | | | GAGGCACATTGACCATTTCCCTTATGTCTGCATTT | |
| | | | | | TCATTTACTGTGCTGTGTATATAGTGTATATAAGCG | |
| | | | | | GACATAGGAGTCCTAATTTACGTCTAGTCGATGTTA | |
| | | | | | AAAAGGTTGCCAGTATATGACAAAAGTAGAATTAG | |
| | | | | | TAAACTACTACATTGAGTACACTTTGTGTTAAAATT | |
| | | | | | CATAGGGAAGACTTCTTAAAAACAAGTGAAATTGT | |
| | | | | | TAAAACCCCCCTAAGCATTACAGATGGCTTATAGC | |
| | | | | | TGTCCACGGGGTTGGTAGAGGTGGGAAAGGGAAGG | |
| | | | | | GTTCTAGGCCAGAATGTTCCTATTTAGAAGACACTC | |
| | | | | | AAATTTACAGTCTGTGTTATGTATGTATACCATTTAT | |
| | | | | | TCAATGCTACTGTGTATATAATGGAAAACTTAAGTC | |
| | | | | | CTGGCGACAGAGCGAGGCTCCGTTTCAAAAAAAAA | |
| | | | | | AGTGCACAATGTAGGTTAACAGTAGAGGGCTTAAG | |
| | | | | | TAACACCCCTCTAAGCATTTGTTTTCAGTACTTCCTA | |
| | | | | | GGAGTGGTTGCATTTGGGAATGGAATTGTTAAAACT | |
| | | | | | TGATGCTTAGGAGCGAATGCAGACTATTCATTGGGT | |
| | | | | | GTTTGGGGTGGGGGAAGGGGGGGTGGGCAGAGGA | |
| | | | | | GGTATGCAGGGAGAGGGGTTCTGTGCTCCTGAGAT | |
| | | | | | TAGTTCAGATGGTCTAACCATTGTTCTATATGTGCA | |
| | | | | | TTTTAGTTAATATTGTGTATTAAAGGATAAGTCTTA | |
| | | | | | ATGCTCAAAGTATGTTTAAAAATAGATGTAGTAAAT | |
| | | | | | CAGTCCCTTTGTGAATGTCCTTTTGTTAGTTTTTAGG | |
| | | | | | AAGGCCTGTCCTCTGGGAGTGACCTTTATTAGTCCA | |
| | | | | | CCCCTTGGAGCTAGACATCCTGTACTTAGTCACGGG | |
| | | | | | GATGGTGGAAGAGGGAGAAGAGGAAGGGTGAAGG | |
| | | | | | GAAGGGCTCTTTGCTAGTATCTCCATATCTAGACGA | |
| | | | | | TGGTTTTAGATGATAACCACAGGTCTACAAGAGCGT | |
| | | | | | TTTTAGTAAAGTGCCTGTGTTCATTGTGGACAAAGT | |
| | | | | | TATTATTTTGCAACATCTAAGCTTTACGAATGGGGT | |
| | | | | | GACAACTTATGATAAAAACTAGAGCTAGTGAATTA | |
| | | | | | GCCTATTTGTAAATACCTTTGTTATAATTGATAGGA | |
| | | | | | TACATCTTGGACATGGAATTGTTAAGCCACCTCTGA | |
| | | | | | GCAGTGTATGTCAGGACTTGTTCATTAGGTTGGCAG | |
| | | | | | CAGAGGGGCAGAAGGAATTATACAGGTAGAGATGT | |
| | | | | | ATGCAGATGTGTCCATATATGTCCATATTTACATTT | |
| | | | | | TGATAGCCATTGATGTATGCATCTCTTGGCTGTACT | |
| | | | | | ATAAGAACACATTAATTCAATGGAAATACACTTTGC | |
| | | | | | TAATATTTTAATGGTATAGATCTGCTAATGAATTCT | |
| | | | | | CTTAAAAACATACTGTATTCTGTTGCTGTGTGTTTC | |
| | | | | | ATTTTAAATTGAGCATTAAGGGAATGCAGCATTTAA | |
| | | | | | ATCAGAACTCTGCCAATGCTTTTATCTAGAGGCGTG | |
| | | | | | TTGCCATTTTTGTCTTATATGAAATTTCTGTCCCAAG | |
| | | | | | AAAGGCAGGATTACATCTTTTTTTTTTTTTTTAGCAG | |
| | | | | | TTTTGAGTTGGTGTAGTGTATTCTGGTTATCAGAAT | |
| | | | | | ACTCATATAGCTTTGGGATTTTGAATTGGTAAATAT | |
| | | | | | TCATGATGTGTGAAAAATCATGATACATACTGTACA | |
| | | | | | GTCTCAGTCCCATAAAATTGGATGTTGTGCCTACAC | |
| | | | | | ACAGGA | |
| 97 | 360 | | 97 | | AAAGTAGTCATTCTTCACTGAGAAGGAACACATAC | |
| | | | | | CAAGGTTAGTGGGTTCGATCATTTGAAAAATGGCA | |
| | | | | | GCACCATTCATTTTAAACATTTTTCTGGCTTTTTACTA | |
| | | | | | TGGAATCTCTCATGGTATAAAAATAAATTTTAGATT | |
| | | | | | TTTCAGAGCCAAAATGAAAATACTTTAGAACAAAA | |
| | | | | | TCAGGCCAAATCTTTGGAATTCAAAGTGGCTGAAC | |
| | | | | | ACCT | |
| 98 | 151 | | 98 | | GGTACCTGAAAGAAAATCAAATAGGAATGACAGTA | |
| | | | | | TTTAGTGTATGGCCAGTGGTTTACTTAGTAACTGGA | |
| | | | | | TGAACAGACTAGAGTTACAGGTTTTGTTTTGTTTTTT | |
| | | | | | TTTTCTATTCCAGTAGTATATCTGAGTAAATCCTGTC | |
| | | | | | CCTCAGTAGATCATCTCTTGGGATCTGGTTTCTTGA | |
| | | | | | TCTGTATTTCAATATATTCTATATTCCATATAGATCA | |
| | | | | | AGACTTTCTAACATAAAGCAGTGTGGAATAGACTT | |
| | | | | | ACTTTTTATCTTCTCTGTTACTCTTTTGATTTGTGAC | |
| | | | | | TTTTACCAATTTATTGAACTTCTTAAGTGTCAGTGTT | |
| | | | | | TTTAATCCATTAGGTTATCGCCAAGGCCTCTAAAAG | |
| | | | | | CTCTAAGATTCAGTGATATGAATACATATTTGCAGT | |
| | | | | | ATTAGAGACATTGTACTGTTTTCACTTGGCTTCTAG | |
| | | | | | GACATTAGATTTTCTATTCTCCCTTTCCTATGCTCAC | |
| | | | | | TCCCAGATTCCTTAACCAGTTCCTTGCATCTTTGTGT | |
| | | | | | ATTAGAATGCCTCAGGGATAAGTCTTGGATTTCTGC | |
| | | | | | TCCTTTCTAGCTGCACTCACTTCCTTGGTAAGCTCAT | |
| | | | | | CTGATTTCATCATAACTTCACCTTTACATACTGCAA | |
| | | | | | ACTCACAAATTATCTTCCCTGAACTTGAGACTCCTA | |
| | | | | | TCCTGCTGCCTGCTTATCATCTTTACTTGACTATATA | |
| | | | | | ACGAACATATCAAACATAAACTGAACTGATAGTCT | |
| | | | | | CCTAACCTGAAACCTGCTTCTATAGTCTTCCCCAAC | |
| | | | | | TAAGTTATTGGCAAATACGTCCTTGCATTTTCTCAG | |
| | | | | | GCCAAAATCACATCATGATCCTTGGCATTTCTTTCT | |
| | | | | | CTGGTACCCCATGCCCTGTCTGCAGATCTATTGGCA | |
| | | | | | AAACCTCCCAACATCTTAACAGCAGCTTTACTACCA | |
| | | | | | CACTTTTCCAAACGGATTACCTCTAGCCTGCATGAT | |
| | | | | | TGCATTAGTCTGCCTCCCTGCTTCTGGCTTTTACCTA | |
| | | | | | CTCAGGCTATTCCCAGCACCCAGAATGACAACTTTG | |
| | | | | | AAAACAAAGCTTGCCGCCACGTGCAGTGGCTCATG | |
| | | | | | CCTGTAATTCCAACGCTTTAGAAGGCGGAAGTGGG | |
| | | | | | CAGATCGCTTGAGGTCAGAAGTTTGAGACCAGCCT | |
| | | | | | GGCCAACATGGTGAAACCCCATCTCTACCAAAAAT | |
| | | | | | AAATAAATTAGCTGGGCATGGTGGTGCATACCTGT | |
| | | | | | AATCCCAGCTACTTGGGAGGCTGAGGCAGGAGAAT | |
| | | | | | CGCTTGAACCTGGGAGGCGGAAGTTGCAGTTAGCA | |
| | | | | | GAGATCATGCCATTGCACTCTAGCCTGGGCGACGG | |
| | | | | | AGTGAGACCCCATCTC | |
| 99 | 17 | | 99 | | TTTTAAGGAAAAAGTGACCTACATTTCATGAAGCA | |
| | | | | | AAGAGATACAGCCACACACAGGAGCCGTTTGTTTT | |
| | | | | | AATTAGATTGCTGGTTTCCCTGGCCAGGACCCAAAA | |
| | | | | | CCACTGTGTTTCCCCATAGATACAATTGACAAATAA | |
| | | | | | AA | |
| 100 | 255 | | 100 | | AGTTCGAGACCAGCCTGGCCAGTATGGCGAAACCC | |
| | | | | | TGTGTCTACTAAATATACAAAAATTAGCTGGAGATG | |
| | | | | | GTGGCAGGCTCCTGTAGTCCCAGCTACACAGGAGG | |
| | | | | | CTGAGGCAGGAGAATCGCTfGAACCTGGGAGGCAG | |
| | | | | | AGGTTGCAGTGAGCTAAGATCGTGCCATTGCACTTT | |
| | | | | | AGTCTGGGCAACAAGAGCAAGACTCCGTCTCAAAA | |
| | | | | | AAAAAAAAAAAAAAAAAAGCCCACAAAAACCAGCA | |
| | | | | | AAAAATCCTCGGCCCCATCACCCCAGTTGCCTCACC | |
| | | | | | AACAGCCTCTCCCAGACCAGGAAGCTGTTTTTATTT | |
| | | | | | TAACTTCATGCAAATGTTGCTAATACAAGATATATT | |
| | | | | | CATTTTTTTAACTTACCCTTTTTTACAAAAAAGATG | |
| | | | | | GTTCTGAAATTGAACTGTATTTAATGTCTTTAATGG | |
| | | | | | TGAAAAAAGGAAAAGTCATAGATGACATGTCATTA | |
| | | | | | TTTTGTAAAATAATAAGATCATGGTCTGGTACTCAC | |
| | | | | | TTTGGCAGCACATATAATAAAATTGGAAAGA | |
| 101 | | Ac025631 | 101 | | AGGTAGTATTTCTGATAATTTTGGACTCATACTCAAA | |
| | | | | | TTCACAAAGTTTTGAAAAGTCATTGTGAATACATTA | |
| | | | | | AGAGAAATAACAGAATCTGACCTGCAAAGACTGCA | |
| | | | | | GATTTTGGAATTACTGGATTAGAGTATTCAAAGACA | |
| | | | | | CACAAAATTTTTTTTAACAACTCTAAAATTCGGATG | |
| | | | | | ACAGTGCAGCATTAAATTGACACAAAATGATGTGT | |
| | | | | | TTTTCAGTACTTGATGGCTTAGATTTATTGAAATAC | |
| | | | | | AGTATGTGTAAGGAATGAAAGAATATCCAAAGATT | |
| | | | | | GAGAAGGAACAAGACAAAAAATGGTGGGGGTGGA | |
| | | | | | GGATAAGCAAGAGCATATGACAAGAAAAATAAGAT | |
| | | | | | TTGGAAACAGTGAAACATCTAGACATGAAAAGCAA | |
| | | | | | AACAGATAAAATGAGCCAGAAGACCAAGATGAAG | |
| | | | | | AAATTATGTAGAATGTATGAAAACTCAACACCAGG | |
| | | | | | GACATTTTGAAAGGTCAATGAACATCTAATAGACT | |
| | | | | | ACCAGAAAGAGATGATAGAATGGTTTGGGATGATA | |
| | | | | | TTTGAGGGTATTTTAGCTGAGAAATTTTCCAATTTG | |
| | | | | | ATGAAAGTCATCCTTGCATTTGAGGAATCAAGAAA | |
| | | | | | ATAATCTGTAGACCCATTGAGCTAATTTGTAGATGA | |
| | | | | | CAGACAACGTGGGAAACCAGAGTGGTGAAACTCTT | |
| | | | | | GATAAGCAAACCACTAAAAATAGTCTCTAAAAGAG | |
| | | | | | CAAGAGAAAGAAAGCATTATCTACAAAGTAACAGC | |
| | | | | | AGTTAGTGTGACAGCTACTTGATAACAATGAAAAA | |
| | | | | | CAGAGGAGAGTGGTATATTTTATGTGCTGAGAAGT | |
| | | | | | GTCAATCTAGAATTCTGTAGCAAACAAAACTATCA | |
| | | | | | GGAAAATGGGCCAAAGACATTTTGGATAAAAAGAG | |
| | | | | | TTTACTACCAACATGTCCTCATTAAATGAACTTAGG | |
| | | | | | AAAGTTTATTCCAGGAATCAGAATTAAGATCAGAA | |
| | | | | | AGAACATGTAAGACGTAAGAAGAGATGGTGAGCAA | |
| | | | | | AGAAAGTGGTAAATGAGGCCAGGCACAGTGGCTCA | |
| | | | | | CACCTGTAATTCCAGCACTTTGGGAGGCCAAGGCG | |
| | | | | | GGCGGATCAACTGAGGTCAGGAGTTCGAGGCCAGC | |
| | | | | | CTGGCCAAGATGGCGAAACCTCATCTCTACTAAAA | |
| | | | | | GTACAAAATTTAGCCAGGCGTAGTGGTGCTTGCTTG | |
| | | | | | TAATCCTGGCTACTTGGGAGGCTAAAGCAGTAGAA | |
| | | | | | TCGCTTGAACCCAGGAGGCAGAAGTTGCAGTGAGC | |
| | | | | | TGAAATTGCGCCACTGCACTCCAGAGCCTGGGCAA | |
| | | | | | CAAGAGCAAAACTCCGTCTC | |
| 102 | 127 | | 102 | 118 | GCGGCCGCGATCCCCACCACACCACCAGCCCGGCC | MELKKSPDGGWGWVIVFVSFLMPFIAQGQGNLINSPT |
| | | | | | GCACGGGGCACTGAGCCGGGTGCTGAGCACCGGAG | SPLAIGLYILKKEVEHHYKKGEMKASLFIKSPYAVQN |
| | | | | | GCCCCGCCGAGGCCGGGACTCAGGACCTGCAGAGA | IRKTAAVGVLYIEWLDAFGEGKGKTAWVGSLASGVG |
| | | | | | AACGCCTCCTGATTTTGTCTTACAATGGAACTTAAA | LLASLGCGLLYTATVTITCQYFDDRRGLALGLISTGSS |
| | | | | | AAGTCGCCTGACGGTGGATGGGGCTGGGTGATTGT | VGLFIYAALQRMLVEFYGLDGCLLIVGALALNILACG |
| | | | | | GTTTGTCTCCTTCCTTATGCCCTTTATTGCTCAAGGT | SLMRPLQSSDCPLPKKIAPEDLPDKYSIYNEKGKNLEE |
| | | | | | CAAGGAAACTTAATTAACAGTCCCACAAGCCCTCT | NINLDKSYSSEEKCRITLANGDWKQDSLLHKNPTVTH |
| | | | | | AGCCATAGGACTGATCTACATCCTCAAAAAGGAAG | TKEPETYKKKVAEQTYFCKQLAKRKWQLYKNYCGE |
| | | | | | TTGAGCACCATTACAAAAAAGGAGAAATGAAGGCT | TVALFKNKVFSALFIAILLFDIGGFPPSLLMEDVARSSN |
| | | | | | AGCCTATTCATAAAATCACCTTACGCAGTACAGAAT | VKEEEFIMPLISIIGIMTAVGKLLGILADFKWINTLYTL |
| | | | | | ATCAGAAAAACAGCTGCTGTTGGAGTCCTGTACAT | YVATLIIMGLALCAIPFAKSYVTLALLSGILGFLTGNW |
| | | | | | AGAATGGCTGGATGCCTTTGGTGAAGGAAAAGGAA | SIFPYVTTKTVGIEKLAHAYGILMFFAGLGNSLGPPIVG |
| | | | | | AAACAGCCTGGGTTGGATCCCTGGCAAGTGGAGTT | WFYDWTQTYDIAFYFSGFCVLLGGFILLLAALPSWDT |
| | | | | | GGCTTTGCTTGCAAGTCTGGATGTGGTTTATTATAC | CNKQLPKPAPTTTTLYKVASNV |
| | | | | | ACTGCAACAGTGACCATTACGTGCCAGTATTTTGAC | |
| | | | | | GATCGCCGAGGCCTAGCGCTTGGCCTGATTTCAACA | |
| | | | | | GGTTCAAGCGTTGGCCTTTTCATATATGCTGCTCTG | |
| | | | | | CAGAGGATGCTGGTTGAGTTCTATGGACTGGATGG | |
| | | | | | ATGCTTGCTGATTGTGGGTGCTTTAGCTTTAAATAT | |
| | | | | | ATTAGCCTGTGGCAGTCTGATGAGACCCCTCCAATC | |
| | | | | | TTCTGATTGTCCTTTGCCTAAAAAAATAGCTCCAGA | |
| | | | | | AGATCTACCAGATAAATACTCCATTTACAATGAAA | |
| | | | | | AAGGAAAGAATCTGGAAGAAAACATAAACATTCTT | |
| | | | | | GACAAGAGCTACAGTAGTGAGGAAAAATGCAGGAT | |
| | | | | | CACGTTAGCCAATGGTGACTGGAAACAAGACAGCC | |
| | | | | | TACTTCATAAAAACCCCACAGTGACACACACAAAA | |
| | | | | | GAGCCTGAAACGTACAAAAAGAAAGTTGCAGAACA | |
| | | | | | GACATATTTTTGCAAACAGCTTGCCAAGAGGAAGT | |
| | | | | | GGCAGTTATATAAAAACTACTGTGGTGAAACTGTG | |
| | | | | | GCTCTTTTTAAAAACAAAGTATTTTCAGCCCTTTC | |
| | | | | | ATTGCTATCTTACTCTTTGACATCGGAGGGTTTCCA | |
| | | | | | CCTTCATACTTATGGAAGATGTAGCAAGAAGTTCA | |
| | | | | | AACGTGAAAGAAGAAGAGTTTATTATGCCACTTAT | |
| | | | | | TCCATTATAGGCATTATGACAGCAGTTGGTAAACTG | |
| | | | | | CTTTTAGGGATACTGGCTGACTTCAAGTGGATTAAT | |
| | | | | | ACCTTGTATCTTTATGTTGCTACCTTAATCATCATGG | |
| | | | | | GCCTAGCCTTGTGTGCAATTCCATTTGCCAAAAGCT | |
| | | | | | ATGTCACATTGGCGTTGCTTTCTGGGATCCTAGGGT | |
| | | | | | TTCTTACTGGTAATTGGTCCATCTTTTCCATATGTGAC | |
| | | | | | CACGAAGACTGTGGGAATTGAAAAATTAGCCCATG | |
| | | | | | CCTATGGGATATTAATGTTCTTTGCTGGACTTGGAA | |
| | | | | | ATAGCCTAGGACCACCCATCGTTGGTTGGTTTTATG | |
| | | | | | ACTGGACCCAGACCTATGATATTGCATTTTATTTTA | |
| | | | | | GTGGCTTCTGCGTCCTGCTGGGAGGTTTTATTCTGC | |
| | | | | | TGCTGGCAGCCTTGCCCTCTTGGGATACATGCAACA | |
| | | | | | AGCAACTCCCCAAGCCAGCTCCAACAACTTTCTTGT | |
| | | | | | ACAAAGTTGCCTCTAATGTTTAGAAGAATATTGGAA | |
| | | | | | GACACTATTTTTGCTATTTTATACCATATAGCAACG | |
| | | | | | ATATTTTAACAGATTCTCAAGCAAATTTTCTAGAGT | |
| | | | | | CAAGACTATTTTCTCATAGCAAAATTTCACAATGAC | |
| | | | | | TGACTCTGAATGAATTATTTTTTTTATATATCCTAT | |
| | | | | | TTTTTATGTAGTGTATGCGTAGCCTCTATCTCGTATT | |
| | | | | | TTTTCTATTTCTCCTCCCCACACCATCAATGGGACT | |
| | | | | | ATTCTGTTTTGCTGTTATTCACTAGTTCTTAACATTG | |
| | | | | | TAAAAAGTTTGACCAGCCTCAGAAGGCTTTCTCTGT | |
| | | | | | GTAAAGAAGTATAATTTCTCTGCCGACTCCATTTAA | |
| | | | | | TCCACTGCAAGGCACCTAGAGAGACTGCTCCTATTT | |
| | | | | | TAAAAGTGATGCAAGCATCATGATAAGATATGTGT | |
| | | | | | GAAGCCCACTAGGAAATAAATCATTCTCTTCTCTAT | |
| | | | | | GTTTGACTTGCTAGTAAACAGAAGACTTCAAGCCA | |
| | | | | | GCCAGGAAATTAAAGTGGCGACTAAAACAGCCTTA | |
| | | | | | AGAATTGCAGTGGAGCAAATTGGTCATTTTTAAAA | |
| | | | | | AAATATATTTTAACCTACAGTCACCAGTTTTCATTA | |
| | | | | | TTCTATTTACCTCACTGAAGTACTCGCATGTTGTTTG | |
| | | | | | GTACCACTGAGCAACTGTTTCAGTTCCTAAGGTAT | |
| | | | | | TTGCTGAGATGTGGGTGAACTCCAAATGGAGAAGT | |
| | | | | | AGTCACTGTAGACTTTCTTCATGGTTGACCACTCCA | |
| | | | | | ACCTTGCTCACTTTTGCTTCTTGGCCATCCACTCAGC | |
| | | | | | TGATGTTTCCTGGGAAGTGCTAATTTTACCTGTTTCC | |
| | | | | | AAATTGGAAAACACATTTCTCAATCATTCCGTTCTGG | |
| | | | | | CAAATGGGAAACATCCATTTGCTTTGGGCACAGTG | |
| | | | | | GGGATGGGCTGCAAGTTCTTGCATATCCTCCCAGTG | |
| | | | | | AAGCATTTATTTGCTACTATCAGATTTTACCACTAT | |
| | | | | | CAAATATAATTCAAGGGCAGAATTAAACGTGAGTG | |
| | | | | | TGTGTGTGTGTGTGTGTGTGTGCTATGCATGCTCTA | |
| | | | | | AGTCTGCATGGGATATGGGAATGGAAAAGGGCAAT | |
| | | | | | AAGAAATTAATACCCTTATGCAGTTGCATTTAACCT | |
| | | | | | TAAGAAAAATGTCCTTGGGATAAACTCCAATGTTTA | |
| | | | | | ATACATTGATTTTTTTTCTAAAGAAATGGGTTTTAA | |
| | | | | | ACTTTGGTATGCATCAGAATTCCCTATAGATCTTTTT | |
| | | | | | GAAAATATAGGTACCTGGGTATCACACATAGAACT | |
| | | | | | TTTAATTCTGCTGGTGTAGGCTGTTGCCCAAACATC | |
| | | | | | TATAATTTTACTGAGCTCTTCAAGTGATTCTGATAA | |
| | | | | | CACAGCCTGGATTGAGAATTTTTATAAGATGGCAA | |
| | | | | | TGGAAAAACATTTATTCTTTTAAATAATAATTTTTTT | |
| | | | | | AAAACCCAAGAGGTCAGGGGATTTTATAAACCAAT | |
| | | | | | AGCCAAGTGTTCTTTAAATAGGAGGCACCCTTCCCA | |
| | | | | | TTGTGCCAAAATCATCTTTTCATTTATTTTGAAATTT | |
| | | | | | GTATGATTATTTTATACTTGTATGTTGCCTTTCTTCG | |
| | | | | | AAGGCGCCTGAAGCACTTTATAAACACAAATCCTC | |
| | | | | | ACAATACCTCTGTGAGGTAGGTAAATAGTACTTTTC | |
| | | | | | TATGTAGTAAACCTGGAATATGGAGAATTTCATAAC | |
| | | | | | AGTTCATTCTACTTAATAATGCAATAATGGAGCTCC | |
| | | | | | AAGTTGTCTTGGACTTCTACACCACACTCAGACTTC | |
| | | | | | TGGAAAGTTTTCTGTACCTCATTCTTTAGTCCCTGTC | |
| | | | | | AAGGTTAGTAAATAAAATAAGTGACATAAAAAAAA | |
| | | | | | AAAAAACTAAACTACTTGTTGTGTTGAAAGTTCCTT | |
| | | | | | TTTGCCAGTTATGTTCAGGAAACCCAATAACCTGAA | |
| | | | | | AAAGTTTGACTTTGATGTGACATCTTCATATTCATC | |
| | | | | | AATGCTGATAATTGTCCAAAGGCATCTTCACTATGT | |
| | | | | | CTGCTAAATAACATCCAATGTGGGCGTTATCTGTTG | |
| | | | | | TCTAGGGGATGAATTTTAAGTTACAATAAAATATTT | |
| | | | | | TTCTTTGTTTTGCA | |
| 109 | AD037 | | 125 | 126 | CTGGCCCCGAGCAGCTGAAGCCTGGGGTCAGCAGG | MKEDCLPSSHVPISDSKSIQKSELLGLLKTYNCYHEGK |
| | | | | | CGCTGCGGGCGCAGCTCCGGTGCAAGCGAGGACAC | SFQLRHREEEGTLIIEGLLNIAWGLRRPIRLQMQDDRE |
| | | | | | GACACATGCAGTGGCTTCTGGACTGCGCGATGACT | QVHLPSTSWMPRRPSCPLKEPSPQNGNTAKGPSIQPV |
| | | | | | GGACGCAAGTAACTTCTAGGTCTGCAGACAAGAGG | HKAESSTDSSGPLEEAEEAPQLMRTKSDASCMSQRRP |
| | | | | | AAGAGAAGATGAAGGAAGACTGTCTGCCGAGTTCT | KCRAPGEAQRIRRHRFSINGHFYNHKTSVFTPAYGSVT |
| | | | | | CACGTGCCCATCAGTGACAGCAAGTCCATTCAGAA | NVRVNSTMTTLQVLTLLNKFRVEDGPSEFALYIVHE |
| | | | | | GTCGGAGCTCTTAGGCCTGCTGAAAACCTACAACTG | SGERTKLKDCEYPLISRILHGPCEKIARIFLMEADLGVE |
| | | | | | CTACCATGAGGGCAAGAGCTTCCAGCTGAGACACC | VPHEVAQYIKFEMPVLDSFVEKLKEEEEREIIKLTMKF |
| | | | | | GTGAGGAAGAAGGGACTCTGATCATCGAGGGGCTC | QALRLTMLQRLEQLVEAK |
| | | | | | CTCAACATTGCCTGGGGGCTGAGGCGGCCCATCCG | |
| | | | | | GCTGCAGATGCAGGATGACCGGGAGCAGGTGCACC | |
| | | | | | TCCCCTCCACCTCATGGATGCCCAGACGGCCTAGCT | |
| | | | | | GCCCTCTAAAGGAGCCATCGCCCCAGAACGGGAAC | |
| | | | | | ATCACAGCCCAGGGGCCAAGCATTCAGCCAGTGCA | |
| | | | | | CAAGGCTGAGAGTTCCACAGACAGCTCGGGGCCCC | |
| | | | | | TGGAGGAGGCAGAGGAGGCCCCCCAGCTGATGCGG | |
| | | | | | ACCAAGAGCGACGCCAGTTGCATGAGCCAGAGGAG | |
| | | | | | GCCCAAGTGCCGCGCCCCCGGTGAGGCCCAGCGCA | |
| | | | | | TCCGGCGACACCGGTTCTCTATCAACGGCCACTTCT | |
| | | | | | ACAATCATAAGACCTCCGTGTTTACTCCAGCCTATG | |
| | | | | | GATCCGTGACCAATGTGAGGGTCAACAGCACCATG | |
| | | | | | ACAACCCTGCAGGTGCTCACCCTGCTGCTGAACAA | |
| | | | | | ATTTAGGGTGGAAGATGGCCCCAGTGAGTTCGCAC | |
| | | | | | TCTACATCGTTCACGAGTCTGGGGAGCGGACAAAA | |
| | | | | | TTAAAAGACTGCGAGTACCCGCTGATTTCCAGAATC | |
| | | | | | CTGCATGGGCCATGTGAGAAGATCGCCAGGATCTT | |
| | | | | | CCTGATGGAAGCTGACTTGGGCGTGGAAGTCCCCC | |
| | | | | | ATGAAGTCGCTCAGTACATTAAGTTTGAAATGCCGG | |
| | | | | | TGCTGGACAGTTTTGTTGAAAAATTAAAAGAAGAG | |
| | | | | | GAAGAAAGAGAAATAATCAAACTGACCATGAAGTT | |
| | | | | | CCAAGCCCTGCGTCTGACGATGCTGCAGCGCCTGG | |
| | | | | | AGCAGCTGGTGGAGGCCAAGTAACTGGCCAACACC | |
| | | | | | TGCCTCTTCCAAAGTCCCCAGCAGTGGCAGGTGTAC | |
| | | | | | ACTGAGCCCTGGTTGCTGGCCCCGGCCGGTCACATT | |
| | | | | | GACTGATGGCCACCGCCTGACGAATCGAGTGCCTG | |
| | | | | | TGTGTCTACCTCTCTGAAGCCTGAGCACCATGATTC | |
| | | | | | CCACAGCCAGCTCTTGGCTCCAAGATGAGCACCCA | |
| | | | | | CAGGAAGCCGACCCAGGCCTGAGGGGCCAGGAACT | |
| | | | | | TGCTGGGTCAGATCTGTGTGGCCAGCCCTGTCCACA | |
| | | | | | CCATGCCTCTCCTGCACTGGAGAGCAGTGCTGGCCC | |
| | | | | | AGCCCCTGCGGCTTAGGCTTCATCTGCTTGCACATT | |
| | | | | | GCCTGTCCCAGAGCCCCTGTGGGTCCACAAGCCCCT | |
| | | | | | GTCCTCTTCCTTCATATGAGATTCTTGTCTGCCCTCA | |
| | | | | | TATCACGCTGCCCCACAGGAATGCTGCTGGGAAAA | |
| | | | | | GCAGGGCCTGCCAGCAGGTATGAGATCTAGCCTGC | |
| | | | | | TTTCAGCCATCACCTTGCCACAGTGTCCCCGGCTTC | |
| | | | | | TAAGCCTCCAATATCACCCTGTGAGCCTCGCACAGC | |
| | | | | | TCAGCCCCAACACAGAGGTGAGACCAGGAATAAGG | |
| | | | | | CCACAAGTATCTCACTTTCTCTGCAGAAATCAATCT | |
| | | | | | TTACTTCATCAGAGAGACCTAAAGCGATTCTTACAA | |
| | | | | | GGAGCTTGCTGCAAGAAACACGGTCATTCAATCAC | |
| | | | | | ATTGAGGAGGGTCCACATGGCATTGAGAGGGTGCT | |
| | | | | | GCCCGCTCAATGCCCAGCAGCAGCTCTGGAAGGCA | |
| | | | | | GTGCTCAGCCCCATCACCACTGTCCCGTGGATGCCT | |
| | | | | | GTGTACCTCTTGCCTTTTCTGGGCTTGCGTTTCTCTC | |
| | | | | | CTCTAGTGGGTGGGGATGACTTTCAATGACTTTCAA | |
| | | | | | TACTTCCCCTGAAGGAAGAATGATAAGGAGAAATG | |
| | | | | | TCTGTTTTGAGGAAAGGGCTTTGAATTCCCCAGATA | |
| | | | | | CTGAACAATTGTGTTTGTGACTGATGGAGAATTTC | |
| | | | | | AGGAATGAATGAGAAAGCCTTTGCGAAACTATGCA | |
| | | | | | ACAGTTTACATCAGTCATGTGAAGTATTTGTCTAAA | |
| | | | | | ACAGAGCAAACTGAAGACCAAATTATTCTCCTGTTG | |
| | | | | | AGGTCCGTGGATGGCAGATTTAAAGGGAAGAACCA | |
| | | | | | CAAAGGCTTGCAAAGATAGGAGAGGCTCCATCTCT | |
| | | | | | AATGCATGTAGAAGCTCCTTACGGGTGCCCATCAA | |
| | | | | | GAGCATAGCTTGGAAGCCACCATGCTGTGCGGAAC | |
| | | | | | TGCGTCAGGGCAAATGTCACAGCAGGATTTCCCCA | |
| | | | | | ACCCAGCTCCATCATCACAGACACAGAGAGCTGCA | |
| | | | | | GGGGAGGCCTGCCCACTGTTTGTCGACTCTGCCCT | |
| | | | | | CCTCTGGCAGCATAGATCCTTAGGTGCTCAATAAAG | |
| | | | | | GTGTGCTGTATTGAACTGAAGAAG | |
| 110 | Cyclin L | NM_02030 | 127 | 128 | CAGTCTTGTTTCGGGTTCCGGCTGCGTTGGGCTTGC | MASGPHSTATAAAAASSAAPSAGGSSSGTTTTTTTTT |
| | | | | | GTGCGGCTCGCTAAGACTATGGCGTCCGGGCCTCA | GGILIGDRLYSEVSLTIDHSLIPEERLSPTPSMQDGLDLP |
| | | | | | TTCGACAGCTACTGCTGCCGCAGCCGCCTCATCGGC | SETDLRILGCELIQAAGILLRLPQVAMATGQVLFHRFF |
| | | | | | CGCCCCAAGCGCGGGCGGCTCCAGCTCCGGGACGA | YSKSFVKHSFEIVAMACINLASKIEEAPRRIRDVINVFH |
| | | | | | CGACCACGACGACGACCACGACGGGAGGGATCCTG | HLRQLRGKRTPSPLILDQNYINTKNQVIKAERRVLKEL |
| | | | | | ATCGGCGATCGCCTGTACTCGGAAGTTTCACTTACC | GFCHVKHPHKIIVMYLQVLECERNQTLVQTAWNYM |
| | | | | | ATCGACCACTCTCTGATTCCGGAGGAGAGGCTCTCG | NDSLRTNVFVRFQPETIACACIYLAARALQIPLPTRPH |
| | | | | | CCCACCCCATCCATGCAGGATGGGCTCGACCTGCCC | WFLLFGTTEEEIQEICIETLRLYTRKKPNYELLEKEVEK |
| | | | | | AGTGAGACGGACTTACGCATCCTGGGCTGCGAGCT | RKVALQEAKLKAKGLNPDGTPALSTLGGTSPASKPSS |
| | | | | | CATCCAGGCCGCCGGCATTCTCCTCCGGCTGCCGCA | PREVKAEEKSPISINVKTVKKEPEDRQQASKSPYNGVR |
| | | | | | GGTGGCGATGGCAACGGGGCAGGTGTTGTTTCATC | KDSKRSRNSRSASRSRSRTRSRSRSHTPRRHYNNRRSR |
| | | | | | GTTTTTTCTACTCCAAATCTTTCGTCAAACACAGTTT | SGTYSSRSRSRSRSHSESPRRHHNHGSPHLKAKHTRD |
| | | | | | CGAGATTGTTGCTATGGCTTGTATTAATCTTGCATC | DLKSSNRHGHKRKKSRSRSQSKSRDHSDAAKKHRHE |
| | | | | | AAAAATCGAAGAAGCACCTAGAAGAATAAGAGAT | RGHHRDRRERSRSTERSHKSKHHGGSRSGHGRHRR |
| | | | | | GTGATTAATGTATTCCACCACCTCCGCCAGTTAAGA | |
| | | | | | GGAAAAAGGACTCCAAGCCCCCTGATCCTTGATCA | |
| | | | | | GAACTACATTAACACCAAAAATCAAGTTATCAAAG | |
| | | | | | CAGAGAGGAGGGTGCTAAAGGAGTTGGGATTTTGT | |
| | | | | | GTTCATGTCAAGCATCCTCATAAGATCATGTTATG | |
| | | | | | TATTTACAAGTCTTAGAATGTGAACGTAATCAAACC | |
| | | | | | CTGGTTCAAACTGCCTGGAATTACATGAATGACAGT | |
| | | | | | CTTCGAACCAATGTGTTTGTTCGATTTCAACCAGAG | |
| | | | | | ACTATAGCTGTGCTTGCATCTACCTTGCAGCTAGA | |
| | | | | | GCACTTCAGATTCCGTTGCCAACTCGTCCCCATTGG | |
| | | | | | TTTCTTCTTTTTGGTACTACAGAAGAGGAAATCCAG | |
| | | | | | GAAATCTGCATAGAAACACTTAGGCTTTATACCAG | |
| | | | | | AAAAAAGCCAAACTATGAATTACTGGAAAAAGAAG | |
| | | | | | TAGAAAAAAGAAAAGTAGCCTTACAAGAAGCCAAA | |
| | | | | | TTAAAAGCAAAGGGATTGAATCCGGATGGAACTCC | |
| | | | | | AGCCCTTTCAACCCTGGGTGGATTTCTCCAGCCTC | |
| | | | | | CAAGCCATCATCACCAAGAGAAGTAAAAGCTGAAG | |
| | | | | | AGAAATCACCAATCTCCATTAATGTGAAGACAGTC | |
| | | | | | AAAAAAGAACCTGAGGATAGACAACAGGCTTCCAA | |
| | | | | | AAGCCCTTACAATGGTGTAAGAAAAGACAGCAAGA | |
| | | | | | GAAGTAGAAATAGCAGAAGTGCAAGTCGATCGAGG | |
| | | | | | TCAAGAACACGATCACGTTCTAGATCACATACTCCA | |
| | | | | | AGAAGACACTATAATAATAGGCGGAGTCGATCTGG | |
| | | | | | AACATACAGCTCGAGATCAAGAAGCAGGTCCCGCA | |
| | | | | | GTCACAGTGAAAGCCCTCGAAGACATCATAATCAT | |
| | | | | | GGTTCTCCTCACCTTAAGGCCAAGCATACCAGAGAT | |
| | | | | | GATTTAAAAAGTTCAAACAGACATGGTCATAAAAG | |
| | | | | | GAAAAAATCTCGTTCTCGATCTCAGAGCAAGTCTCG | |
| | | | | | GGATCACTCAGATGCAGCCAAGAAACACAGGCATG | |
| | | | | | AAAGGGGACATCATAGGGACAGGCGTGAACGATCT | |
| | | | | | CGCTCCTTTGAGAGGTCCCATAAAAGCAAGCACCA | |
| | | | | | TGGTGGCAGTCGCTCAGGACATGGCAGGCACAGGC | |
| | | | | | GCTGACTTTGTCTTCCTTTGAGCCTGCATCAGCTT | |
| | | | | | GGTTTTGCCTATCTACCAGTGTGATGTATGGACTCA | |
| | | | | | ATCAAAAACATTAAACGCAAAACTGATTAGGATTT | |
| | | | | | GATTTCTTGAAACCCTCTAGGTCTCTAGAACACTGA | |
| | | | | | GGACAGTTTCTTTTGAAAAGAACTATGTTAATTTTT | |
| | | | | | TTGCACATTAAAATGCCCTAGCAGTATCTAATTAAA | |
| | | | | | AACCATGGTCAGGTTCAATTGTACTTTAATTAGTT | |
| | | | | | GTGTATTGTTTATTGCTATAAGAACTGGAGCGTGAA | |
| | | | | | TTCTGTAAAAATGTATCTTATTTTTATACAGATAAA | |
| | | | | | ATTGCAGACACTGTTCTATTTAAGTGGTTATTTGTTT | |
| | | | | | AAATGATGGTGAATACTTTCTTAACACTGGTTTGTC | |
| | | | | | TGCATGTGTAAAGATTTTTACAAGGAAATAAAATA | |
| | | | | | CAAATCTTGTTTT | |

TABLE III

| Gene No | Clone Name | NT SEQ ID. No. X | Total NT Seq of Clone |
|---|---|---|---|
| 103 | 36d5 | 103 | 536 |
| 103 | 36d5 | 283 | 1072 |
| 104 | 37e4 | 104 | 862 |
| 105 | 35e2 | 105 | 1072 |
| 106 | 42e7 | 106 | 856 |
| 107 | 105b2 | 107 | 1155 |
| 108 | 41h1 | 108 | 3344 |

TABLE IV

| | | | NT | AA | | |
|---|---|---|---|---|---|---|
| | | Genbank | SEQ | Seq | | |
| Gene | Clone | Accession | ID. | ID | | |
| No. | Name | No. | No.X | No.Y | Polynucleotide Sequence | Polypeptide Sequence |
| 103 | 36d5 | | 103 | | CTGGGAGACACGTCAGGGAGAGGTAGCTGTGGTCA | |
| | | | | | CTGCCTTGTACAACAGCCAAAAGCCCAAAGCAGGA | |
| | | | | | GGGACCCTGGCCTTTCTCCCAGCACACAACGAGTGG | |
| | | | | | GAGCTCTGTGTGCTGGCCGGCATTTCCTGTCACGTT | |
| | | | | | CAATAGGACACGTTCACTCTTCATACTTCTTCAATT | |
| | | | | | CTAAATTTCAGAAGTAATTTGTCACTTTAGAGGAGG | |
| | | | | | GCGTCATTAATAACCATTATTTAGAACTGTCGAGTC | |
| | | | | | TTCCTCTCTGTGAGTGTCTGAGTTAAGCATCCCCAA | |
| | | | | | AATTGGCCTTGTTGGTGGCAAGCAGTGCCCCCACAC | |
| | | | | | TGACAGATTGAGACTACCCCACCCCCACCGACGCC | |
| | | | | | CTCACAACCCAGTTCTTCCCCGTCTGCCTTTAATCA | |
| | | | | | CCGCGAGGGGGGCGACAGGGAATGGCTACGGCATG | |
| | | | | | TCCTCCTGGAATTCATTAGCGTTATTACCAAAGACC | |
| | | | | | GTGTTGTAAATTGAGATTTTTTTTAACTGCTAGGAA | |
| | | | | | AAAATTTCTCCTTAACTATTTCATTTTATTGTACTTA | |
| 103 | 36d5 | | 283 | | AAAATGTACTTAGAAATTTTAAAAGCACAAAACAA | |
| | | | | | ACGCATTCTCTCCCCATCCTCCTATCTCCAGCTCTTA | |
| | | | | | GAGACTGGAGCTCAGCACCTAAGCTGTTAATGAAT | |
| | | | | | GGGGACAGCTTTCATCTCCACTGGAAAAAAGCCTG | |
| | | | | | CTCTCTCACTTGGGGTCCCTCTCCCCCTTCCACTTGC | |
| | | | | | ATTCAATCAGCACCCATGCAACCATCCTCCCTGCTC | |
| | | | | | TGAGCTCTGTGAGCCCCTGAAAATAGAGAAATTGG | |
| | | | | | GTGTTTGTGGAGCAAAATATAGCTAAGTAATTTTTC | |
| | | | | | CTGCTCCTTTGAGGCCATGTTCTTTCATGGTGAGGG | |
| | | | | | AGGGGCAGAGAAAATAGAGGCTCACAAATCCCTTT | |
| | | | | | TCCTGTGACTCCCACAACTTAGGCCAGGGGCCTTCT | |
| | | | | | TGAGCCTCATAATGTGTGTGTGTAGATAGGGGAAA | |
| | | | | | GGAGGTCCACTTCCAGAATTTTCCCTGTGTTCTTATT | |
| | | | | | CCTCACTTATGCTACCGTTGGCTCAGCTGGCCCGAA | |
| | | | | | CCAAGATCCATAGCCAGGTTTCCATCACTGATGAGC | |
| | | | | | TCCCCAAAACAGGGTGACCTTCCCCTCCTCGTGGGG | |
| | | | | | TAAGGAAAGCTCTCATATCATTGGACTTCAGGCAG | |
| | | | | | GAAGGGTCAGTTGGAAAGAAACCTTTGACGTGAGC | |
| | | | | | CTCTTGATGTCTCCATGGCCTCTGTGCCTCCATGCTG | |
| | | | | | GCCCAGGCCTTCTGTGCTTATGCCCAGGAAGCATGT | |
| | | | | | GGCCAGTGAATGAATGCACCCAGGATGCCTCCTTCT | |
| | | | | | TTTCCATGGGAGCCCAGAAGATGCCACTTGGAGCTC | |
| | | | | | AGCGTCCTGGTGTCTAGAAAAGTTTCTGGTGCCAGC | |
| | | | | | AGTGCTGCTCCATTTGGTACAGCAGGTGCCAAGCCT | |
| | | | | | CTCAATGGAGGCTCTTTGGACTTCTATGAAAAATTA | |
| | | | | | TTAATGAGCTTCCAGACTTTCATATCTGGCATTTATT | |
| | | | | | CTCCAATGGATACCTGAGGAAAAACCTTTTTCTTCA | |
| | | | | | TCAAATAGAACTTGAGGAGAAATCAAAAAGACAAC | |
| | | | | | TTCAGGAGGCAACAGATGGGAAGTGCCTGCCTTTA | |
| | | | | | AACAAAACAAAACATAAACAGGCTTTATGCCTT | |
| 104 | 37e4 | | 104 | | CCTGCCTCGACAAAATTAAAAAAATAAGTATTGTTG | |
| | | | | | CTCCCCTTTTGGAGATGAGTCAAAAAGATTAAACA | |
| | | | | | ACTAGCCCCAAGTCATGGAGATAATTAAAAAAGAT | |
| | | | | | TAAACAACTAGCCCCAAGTCATGGAGATAATTAAA | |
| | | | | | AAAGATTAAACAACTAGCCCCAAGTCATGGAGATA | |
| | | | | | AAAAGGTCAGAATTTCTTTTTTAGAAACGGGGTCTT | |
| | | | | | ACTCTGTTGCCCAGTCTGGAGTGCAATGGCACAGTC | |
| | | | | | ATGGCTCAATGTAACCTCAGACTCCTGGGCTCAAGC | |
| | | | | | GGTCCTCCCACGTGAGCCTCCCAAGACTACAGGTGC | |
| | | | | | ACACCATCATACACGGGACAGGGTCTCGCTATATTG | |
| | | | | | CTCAGACTGGAAAGTTCAGATTTTTAAATCAGGTCT | |
| | | | | | TGGGACTCCCGATTCTGTTTTTCCACAGAGTCACCA | |
| | | | | | TCTATCCTGACAATGCTCCATTTCATGCTGTTTTTCC | |
| | | | | | TCACCTTCAATACTGCTCCCCCATCCCCCCACCTCT | |
| | | | | | AGGTGTGAAGGTTACCAGGAGAGACCTGAGCTCGC | |
| | | | | | TGGCTCTGACTCCAAGGTGGCCTCAGTGGAAAGTTT | |
| | | | | | CAAAAGGCAACCGGTTTGGTTTCACTGGCAGGGCA | |
| | | | | | GCGGCAGGCGTTTGGGTTCTGGAGGCCCAGGAATG | |
| | | | | | TAGAAGCCTCCAGCTAACAGACTCCACGCGCCTATC | |
| | | | | | CTCCCAAACGCTCTCGGAGATAAGCTCCCAGCTCCC | |
| | | | | | TCCCCTTTTCCACCTTCATGCACTTCCTGCTGTATTC | |
| | | | | | TGTCCATTCCAGCACTGGCCCTTTCTGTGGGTGGGT | |
| | | | | | GGGCAGAGGATACAATTTCCTGCATGACTACTTGCT | |
| | | | | | CATGATTCATACTTCTAAATGAAAGTACAACTGATA | |
| | | | | | TAA | |
| 105 | 35e2 | | 105 | | AAATGTACTTAGAAATTTTAAAAGCACAAAACAA | |
| | | | | | ACGCATTCTCTCCCCATCCTCCTATCTCCAGCTCTTA | |
| | | | | | GAGACTGGAGCTCAGCACCTAAGCTGTTAATGAAT | |
| | | | | | GGGGACAGCTTTCATCTCCACTGGAAAAAAGCCTG | |
| | | | | | CTCTCTCACTTGGGGTCCCTCTCCCCCTTCCACTTGC | |
| | | | | | ATTCAATCAGCACCCATGCAACCATCCTCCCTGCTC | |
| | | | | | TGAGCTCTGTGAGCCCCTGAAAATAGAGAAATTGG | |
| | | | | | GTGTTTGTGGAGCAAAATATAGCTAAGTAATTTTTC | |
| | | | | | CTGCTCCTTTGAGGCCATGTTCTTTCATGGTGAGGG | |
| | | | | | AGGGGCAGAGAAAATAGAGGCTCACAAATCCCTTT | |
| | | | | | TCCTGTGACTCCCACAACTTAGGCCAGGGGCCTTCT | |
| | | | | | TGAGCCTCATAATGTGTGTGTGTAGATAGGGGAAA | |
| | | | | | GGAGGTCCACTTCCAGAATTTTCCCTGTGTTCTTATT | |
| | | | | | CCTCACTTATGCTACCGTTGGCTCAGCTGGCCCGAA | |
| | | | | | CCAAGATCCATAGCCAGGTTTCCATCACTGATGAGC | |
| | | | | | TCCCCAAAACAGGGTGACCTTCCCCTCCTCGTGGGG | |
| | | | | | TAAGGAAAGCTCTCATATCATTGGACTTCAGGCAG | |
| | | | | | GAAGGGTCAGTTGGAAAGAAACCTTTGACGTGAGC | |
| | | | | | CTCTTGATGTCTCCATGGCCTCTGTGCCTCCATGCTG | |
| | | | | | GCCCAGGCCTTCTGTGCTTATGCCCAGGAAGCATGT | |
| | | | | | GGCCAGTGAATGAATGCACCCAGGATGCCTCCTTCT | |
| | | | | | TTTCCATGGGAGCCCAGAAGATGCCACTTGGAGCTC | |
| | | | | | AGCGTCCTGGTGTCTAGAAAAGTTTCTGGTGCCAGC | |
| | | | | | AGTGCTGCTCCATTTGGTACAGCAGGTGCCAAGCCT | |
| | | | | | CTCAATGGAGGCTCTTTGGACTTCTATGAAAAATTA | |
| | | | | | TTAATGAGCTTCCAGACTTTCATATCTGGCATTTATT | |
| | | | | | CTCCAATGGATACCTGAGGAAAAACCTTTTTCTTCA | |
| | | | | | TCAAATAGAACTTGAGGAGAAATCAAAAAGACAAC | |
| | | | | | TTCAGGAGGCAACAGATGGGAAGTGCCTGCCTTTA | |
| | | | | | AACAAAACAAAACATAAACAGGCTTTATGCCTT | |
| 106 | 42e7 | | 106 | | CAGTTTCATGTGCTTAAGCAACTTTGCTTCAGGTCA | |
| | | | | | CACCCTACGGGACACCCACGGCAGCCTGCCGCCTA | |
| | | | | | CTAATCATAGAGCCCTTCGTGTTCCTTTTTTGTCTTT | |
| | | | | | TTCTTAACCAACAATGGGTCATTTAGCAGGACATTT | |
| | | | | | ATTTCAGTCCTAAGTTGTATTATCCCTGGTAATTTGC | |
| | | | | | ATATACCATTATTAAAGTGTGGCAGTCTTTTGTAAT | |
| | | | | | TATTGTCTTAATCTAGTGAAAAATAATATATCTGTA | |
| | | | | | TATCTGGAGAGAAGGCTGTTCTCTGGATGCAGCTGA | |
| | | | | | GCACTTGCATGCACTCGATGAACGGGAATAGGACT | |
| | | | | | GCATGAGGCTGACCTGGATTTGACAACCGCACCAG | |
| | | | | | GACAAGGCCGCGTGCTGCCCTGAACAGTGGCCCTT | |
| | | | | | GTGCTAAATACGAATCCTCTCTCTCCCACAGGCATA | |
| | | | | | GCCCGTCACCTGCGTCTGGTTTTTGCTCCTCATTTTC | |
| | | | | | TTCAATTTTCACTCTATTTATAGTTGAGAACCTTCCA | |
| | | | | | TTTCCCCCTGGTTGAAATACATTAGTTGCTATGGAA | |
| | | | | | ACTGCGATCCCCCCGGTGTGGATGGAGCTGAATGA | |
| | | | | | CACCTACAATTGCAGAGCACGGTTGGCGTTGCCAG | |
| | | | | | GGCTGGGAAATGGGCGTCGTGGCTGGAGAGGGCAC | |
| | | | | | TGAAGGGCACAGATGAGAATAATGACAGCACACAG | |
| | | | | | CACGACCGTCAGGAACCGACGCAGCACCACTGGGT | |
| | | | | | CAGAAGTTGTGGAAGAAGCCATGGGTAACAGAAGC | |
| | | | | | CCCCCATGCCCTACACCACACAGAGGGGCGGGTCC | |
| | | | | | CATCAGAGGCCTAACCCCTGGAGGGCTCTCATTTTC | |
| | | | | | AAAACATAAAAAATGGAGCTATAGCTGGTACTTGC | |
| 107 | 105b2 | | 107 | | GAGTCCTAATTAGGGAAAAGGAGTCAGGCTGGTGG | |
| | | | | | GACCAAGGAAAAGCAAAGAGAAAGCACATAAGCT | |
| | | | | | GTAAGTCTGCCTTTCTTCATGGTCCAGGACACATAA | |
| | | | | | TCCTCCTGCGTAAATAAGTCACAATCTTCCTGCGCC | |
| | | | | | CAGCTATCATCAGACCCTCGGCTGATAGAAAAATG | |
| | | | | | CAAATTAGCTCACTGCAACCTTGGCATTATCAGTAC | |
| | | | | | TGCACATAGCTCTCTCCAGAAAACAGCACGAACAC | |
| | | | | | CATCCTATAAAATCCACAGCAAGCCTTTGTCTCCTC | |
| | | | | | ACAGTCAGCTCCCTTCTTTCTGACTTGCCCACTGCTT | |
| | | | | | TCTTGCAACGCAATTTCATACTTGTGATTCTTATGCC | |
| | | | | | TCAGCCATCCAAGTAGCTGGGATTACAGCATGCGCT | |
| | | | | | ACCACACCTGGCTTTTTTATTATTATTATTTTTGGAG | |
| | | | | | AGATGACATCTTTGCTATGTTGTCCAGGCCACTCTCA | |
| | | | | | AACTCCTGGAATAAAGGGATCCTCCCACATTGGCCT | |
| | | | | | CCCAAAGTGCTGGGATTATAGATAGGTGTGAACCA | |
| | | | | | TCATACCCAGCTTTATTTTATTTTTTTGTAGAGATAG | |
| | | | | | GGGTCTCGTTCACTTGCCCAGGCTGGAATGTCCGGT | |
| | | | | | TTTACTTTCCTGTTTTTTCTTGGTGGCAGATACCATT | |
| | | | | | TGTTTGCTTTCAGATATAACATTCCCCTAAGCACTT | |
| | | | | | CTTGTAGGCCGAGTCTAGTGGTGATGCATTCCCTCA | |
| | | | | | GTTTTTACTTGTCTGGGAAACACTTTATTTCTCCTTT | |
| | | | | | TCTTCTTCAGGGAGTTTTAATTTTTCTTAAACATGTG | |
| | | | | | GTCACTCTCTAGAGGTGGGACACCCCCACCCCATTT | |
| | | | | | TTGGCTTAGATCTTCTCGTGTGTCGACTTGTGTCCCC | |
| | | | | | CTAGAAGGAGTGTTGAAGTCCTAACCCACAGTACC | |
| | | | | | TGTGATTGTGATCTTTTTTTGAGATAGGGTGTGATTT | |
| | | | | | CTAAAAAATTATATGTGATTAGTTAAAATGAGTTCA | |
| | | | | | CAGTGGATTAGGGTGGGTTCACATATAATAAGACT | |
| | | | | | GATATTCTTTACAAGAAGTGGAGAAGAGACCCAGAG | |
| | | | | | GGGAGAAAGCCATGTGAAGACAGAGGTGGAAGCT | |
| | | | | | GGTGTAGCTGTCAAGCCACACATACACTGTATTAGT | |
| | | | | | TTCCTGTGGTTCCTGTCAAAGGTACCACAAACTGGT | |
| | | | | | GAGTT | |
| 108 | 41h1 | | 108 | | GGACCGGCTCCGGACCGCGCAGTTAGCGCCGCCTG | |
| | | | | | GCCTGGGCCGGACCCGGTCAGGGTTCTCAAGCTGTC | |
| | | | | | GTCCCTATGGGGCTGTGTTTTCCTTGTCCCGGGGAG | |
| | | | | | TCCGCGCCTCCCACGCCGGACCTGGAAGAGAAAAG | |
| | | | | | AGCAAAGCTTGCAGAGGCTGCAGAGAGAAGACAA | |
| | | | | | AAAGAGGCTGCATCTCGGGGAATTTTAGATGTTCA | |
| | | | | | ATCTGTGCAAGAAAAGAGAAAGAAAAAGGAAAAA | |
| | | | | | ATAGAAAAACAAATTGCTACATCCGGGCCCCCACC | |
| | | | | | AGAAGGTGGACTTAGGTGGACAGTTTCATAAAGCA | |
| | | | | | TAACATGAGTAGAAGAATCTACTGCCAATAACTGTT | |
| | | | | | TATTATCTGCAATCAAGTGGGCTTCATCAATTTAAT | |
| | | | | | TTCTTCTCTTTGAGTAAATGAAGATTCAGACTTTGT | |
| | | | | | AATATTATTGCCCTTAAGTGCAATGCTAAAAAAACG | |
| | | | | | TTGATTTTCAAGCTTAGAGAATGGCTAGACTTTTCA | |
| | | | | | TTAAATACTGATTTCCTACATTTGCTCTTCTGCAGT | |
| | | | | | TAGTGGGTGATTTGCTATTTTTCTTAGTAGTTAAAA | |
| | | | | | AATGGAACTAAATAGTGAATATACATACACTGCAT | |
| | | | | | GTAAACATTCTGCATATACCTCTAAGATTAAAATTC | |
| | | | | | GCAGTTGTCTTTTCATCCTTATAAAATGATCTAACT | |
| | | | | | ACTTATATTTGTGCTGCATCGCGTTACATCTGTTTTT | |
| | | | | | ATTTCACTATGAAGATGTTTGATTAAACTTATGGAC | |
| | | | | | TTAGTGCCTTTAAACTGATCATCAGGGAGAATCTTG | |
| | | | | | AAAAAATCATTTGAAGGGCTGATGTGAAGGAGCAC | |
| | | | | | TGTAAATTTTTATAACTTAGTAATGAGTATTCTTAG | |
| | | | | | GCAGATGTAAAATTTTTTCCAATTTATTTTTATTTAT | |
| | | | | | GTAGCTTATAAAATTAACATACCCTGTTTTACTTTA | |
| | | | | | TGATAAAGGATTTTTTGTTTGCTGAATTTAAAATTA | |
| | | | | | TATATTAGTGATACCATCAGAGGGCAGTGATGTTCT | |
| | | | | | ATTGTATATTAAATTCAGCTCTGTAAGGATCTTTGT | |
| | | | | | AGTAATTGAATGAGTTAAACTAATAATCTGGATGG | |
| | | | | | GTTATAATGAGTAGTAATATATTTGTCCATATTTCA | |
| | | | | | TAAGTAGTGTTAATCTTGTGTACTTATTAGAGAACG | |
| | | | | | ATCATAAGATTTATACAGATGTGAAACTGCGAAGG | |
| | | | | | CAAGTATGAATGTATGAAAAAAACATGTAGGTACT | |
| | | | | | GTACTTACAAAAGGTCTACTTCAGATATAAAAATAT | |
| | | | | | TAGGTAATTCTATACAATGCATAGTCATAAACCTTA | |
| | | | | | ACATTTTTGTTTCATTAGAAACATGAATTTTATAGCA | |
| | | | | | TTTTTTGTTTCTCCTATATAATACACTGAAATAAAA | |
| | | | | | GAATTTGTGTTAGCTATTAAGGCTGATAGCTCTTTT | |
| | | | | | AAATGGCAAGGCCACATGTTGAGCCCTAAATTAAA | |
| | | | | | ATTTGCAGATATTAAGTGCTAATAGAAATTTTAAGT | |
| | | | | | TAAATCGACCAAGTTCACTTGCTTTACACAAAGGAA | |
| | | | | | ACTGAGCCACTATCTTCATCTACCCCTCCAACAAAA | |
| | | | | | ATTATGTTATACTGCAGTGTATTGTACATGTTAATTT | |
| | | | | | TTAAAAGTTTGAACTATTATATAATACAGGTCTCTT | |
| | | | | | GACTTCTCATGGAAAAATTATTTTTTCTATTATGGT | |
| | | | | | GTGAAATATTGTGTGAATATCTAGGCAAAACATAA | |
| | | | | | CAATTTGGCTCAATTTTCTTCTTTAGAGGATTCGTGC | |
| | | | | | TGTTTTGTTCATAAAGGGTAGTGAAATCATTGAAC | |
| | | | | | TATATTTTAGAATGAAAATTTTTGATTTTTAAAA | |
| | | | | | TGATTTTTTCAAGGCAGAAAGTAAAAGGAATGATT | |
| | | | | | GATAGCGGAGTGCATATAGAGCTAGAGCATATCAT | |
| | | | | | CCTTGAACTCTGCAAATCCTTTCTTCCATTTTAATAT | |
| | | | | | AGCAAGAACAATTTTGTCTTTACTACATCTTAAAGA | |
| | | | | | ATTAGAACTTGGGTTGGTGTAAGTGACTTACTTCCA | |
| | | | | | GGGAATCATGCCCTATTTCTACCAGCAGGTCATACC | |
| | | | | | CAAATGTCACACTATCTATTGTTAACCATGAATGAT | |
| | | | | | ATTCAGATCTATTACTTTTCGTGAAAAGTGGAACAT | |
| | | | | | GTTACTTCCAACCATGGCCTGTCACCGTGAGTGTGA | |
| | | | | | TCAGCTTTCTCCAAAACCACATGGGTCGCAGGAGCT | |
| | | | | | AAGGGGTGGTACCCAAATGTTAGGAACAGTGTTAG | |
| | | | | | GAAAGGGCAAGGGAAAAGAAGTGACTGGATGTCTT | |
| | | | | | ATGAGAAACCGGTAAATGACTAAAAAAAAAAGCA | |
| | | | | | AATGACTAAAAACATGACTAAAAAATTATATATAT | |
| | | | | | ATATAATATATATATTATATATGTGTGTATATATAT | |
| | | | | | ACACATAATATCTGCAAATTCTAATTTATATATGTG | |
| | | | | | TGTGTATATACACACACACACATGCACATACACAC | |
| | | | | | ATACGTCCAGACATCTCCCTCATAAAATAACCATCA | |
| | | | | | GTTTCTATGAAAACCTTAAGTGGAAGCCAATTTCCC | |
| | | | | | ATAGTAAATAATTTAGGAGAAAATTATAATGCTTA | |
| | | | | | AAATGTTGCTCAAACCCCTGACCTATTACTAAACTA | |
| | | | | | TAATTGGAACAGTAAAATGCATATATGTAACTATCA | |
| | | | | | TATCATGATTTAAAATTGCTTAAACCATTGCTGCTT | |
| | | | | | AATACTAATCAAACTTAACGGCTGCTAACAAAAGT | |
| | | | | | TGTGAATTATTACACGGCCTCTTTGTAACGTGCTGC | |
| | | | | | ATGTTTTTAAAACATCTCTGTGTTTCTGTTTGTTCC | |
| | | | | | ACTGCTGGTATTTGGAATGTAATTTAACAGTTCTCA | |
| | | | | | CACATGGTTTGGTTATAAATTCTGTATTGCCTTTTAG | |
| | | | | | GGATATAAATATACATTTTTTTCTATGTAAAAATTA | |
| | | | | | GCTTTAGCTGTCTCTTTAACAAAATTTTATCTTTACT | |
| | | | | | ACATCCTAAATACTTAGAACCTGAGTTGGTGGTTAG | |
| | | | | | GGAAACCTCAGGAACATTTTAATCACATTGGGATTC | |
| | | | | | AGAAGAGCAACAGAACCAAAGGTTGTTTGGTGTGT | |
| | | | | | TCATACAATCCCTGGATTTATAGGTGGATTTTCTAT | |
| | | | | | AAAGGAAAAATGATGTAATTAGTATCCTGTTTTTTC | |
| | | | | | CTAAAGAAATAATACTATCATAAAAATTCTGTCTAT | |
| | | | | | CCTTTGTACCCCAGGAAAATGGACATGAACTTTGAA | |
| | | | | | TTTTCCCTTTCTCCAAATGTTTGACTTTTTATTTTCA | |
| | | | | | CTGATAAGCATTATGCTATGTTCTTAGAAGACAAAA | |
| | | | | | GCAGCTCTTGCCAGTTTTGAATAATTTCTGCATGAA | |
| | | | | | TAGACCAGTAAGAGGTAAGTAGCCATGACTGCCTA | |
| | | | | | TATGTGTTGAGACATAAGGTATATTTCTTTAACATC | |
| | | | | | TCCAAGCAAGCATTTCAAATTCTCTTAACTACTAAA | |
| | | | | | CATGCTCTAAGCT | |

What is claimed is:

1. An isolated nucleic acid molecule consisting of a polynucleotide having a nucleotide sequence selected from the group consisting of:
(a) a polynucleotide fragment of SEQ ID NO:1-108, 125, 127, 132-140, 158-159, or 264-284;
(b) a polynucleotide encoding a polypeptide fragment of SEQ ID NO:109-118, 126, 128, 144-152, or 160-161, which is hybridizable to SEQ ID NO:1-108, 125, 127, 132-140, 158-159, or 264-284;
(c) a polynucleotide encoding a polypeptide domain of SEQ ID NO:109-118, 126, 128, 144-152, or 160-161 which is hybridizable to SEQ ID NO:1-108, 125, 127, 132-140, 158-159, or 264-284;
(d) a polynucleotide encoding a polypeptide epitope of SEQ ID NO:109-118, 126, 128, 144-152, or 160-161, which is hybridizable to SEQ ID NO:1-108, 125, 127, 132-140, 158-159, or 264-284;
(e) a polynucleotide encoding a polypeptide of SEQ ID NO:109-118, 126, 128, 144-152, or 160-161 which is hybridizable to SEQ ID NO:1-108, 125, 127, 132-140, 158-159, or 264-284, having NFkB modulating activity;
(f) a polynucleotide which is a variant of SEQ ID NO:1-108, 125, 127, 132-140, 158-159, or 264-284;
(g) a polynucleotide which is an allelic variant of SEQ ID NO:1-108, 125, 127, 132-140, 158-159, or 264-284;
(h) a polynucleotide which encodes a species homologue of the SEQ ID NO:109-118, 126, 128, 144-152, or 160-161;
(i) a polynucleotide which represents the complimentary sequence (antisense) of SEQ ID NO:1-108, 125, 127, 132-140, 158-159, or 264-284;
(j) a polynucleotide capable of hybridizing under stringent conditions to any one of the polynucleotides specified in (a)-(i), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment consisting of a nucleotide sequence encoding a NFkB modulatory protein, or fragment thereof.

3. The isolated nucleic acid molecule of claim 1, wherein the polynucleotide fragment consisting of a nucleotide sequence encoding the sequence identified as SEQ ID NO:109-118, 126, 128, 144-152, or 160-161, which is hybridizable to SEQ ID NO:1-108, 125, 127, 132-140, 158-159, or 264-284.

4. The isolated nucleic acid molecule of claim 1, wherein the polynucleotide fragment consisting of the entire nucleotide sequence of SEQ ID NO:1-108, 125, 127, 132-140, 158-159, or 264-284, which is hybridizable to SEQ ID NO:1-108, 125, 127, 132-140, 158-159, or 264-284.

5. The isolated nucleic acid molecule of claim 2, wherein the nucleotide sequence consisting of sequential nucleotide deletions from either the C-terminus or the N-terminus.

6. An isolated polypeptide consisting an amino acid sequence selected from the group consisting of:
(a) a polypeptide fragment of SEQ ID NO:109-118, 126, 128, 144-152, or 160-161;
(b) a polypeptide fragment of SEQ ID NO:109-118, 126, 128, 144-152, or 160-161, capable of modulating an NFkB response;
(c) a polypeptide domain of SEQ ID NO:109-118, 126, 128, 144-152, or 160-161;
(d) a polypeptide epitope of SEQ ID NO:109-118, 126, 128, 144-152, or 160-161;
(e) a full length protein of SEQ ID NO:109-118, 126, 128, 144-152, or 160-161;
(f) a variant of SEQ ID NO:109-118, 126, 128, 144-152, or 160-161;
(g) an allelic variant of SEQ ID NO:109-118, 126, 128, 144-152, or 160-161; and
(h) a species homologue of SEQ ID NO:109-118, 126, 128, 144-152, or 160-161.

7. The isolated polypeptide of claim 6, wherein the the full length protein consists sequential amino acid deletions from either the C-terminus or the N-terminus.

8. An isolated antibody that binds specifically to the isolated polypeptide of claim 6.

9. A method for preventing, treating, or ameliorating a medical condition, comprising administering to a mammalian subject a therapeutically effective amount of the polypeptide of claim 6.

10. A method of diagnosing a NFkB associated condition or a susceptibility to a NFkB associated condition in a subject wherein said condition is a member of the group consisting of an immune disorder; an inflammatory disorder in which polypeptides of the present invention are associated with the disorder either directly; or indirectly; an inflammatory disorder related to aberrant NFkB regulation; a cancer; aberrant apoptosis; hepatic disorders; Hodgkins lymphomas; hematopoietic tumors; hyper-IgM syndromes; hypohydrotic ectodermal dysplasia; X-linked anhidrotic ectodermal dysplasia; Immunodeficiency; al incontinentia pigmenti; viral infections; HIV-1; HTLV-1; hepatitis B; hepatitis C; EBV; influenza; viral replication; host cell survival; and evasion of immune responses; rheumatoid arthritis inflammatory bowel disease; colitis; asthma; atherosclerosis; cachexia; euthyroid sick syndrome; stroke; EAE; autoimmune disorders; disorders related to hyper immune activity; disorders related to aberrant acute phase responses; hypercongenital conditions; birth defects; nercrotic lesions; wounds; organ transplant rejection; conditions related to organ transplant rejection; disorders related to aberrant signal transduction; proliferating disorders; cancers; HIV propagation in cells infected with other viruses, associated with IL-8, disorders associated with aberrant IL-8 expression, disorders associated with aberrant IL-8 activity, asthma, pulmonary disorders, pulmonary fibrosis, Behcet's disease, bacterial infections, viral infections, gynaecological diseases, psoriasis, inflammatory bowel disease, IgA nephropathy, chronic obstructive pulmonary disease, Kawasaki disease, Crohn's disease, peripheral arterial occlusive disease, Hodgkin's disease, idiopathic intermediate uveitis, hyaline membrane disease, acute rheumatic fever, chronic rheumatic heart disease, ulcerative colitis, autoimmune disorders, and autoimmune thyroid disease; comprising:
(a) determining the presence or absence of a mutation in the polynucleotide of claim 1; and
(b) diagnosing a NFkB associated condition or a susceptibility to a NFkB associated condition based on the presence or absence of said mutation, wherein said mutation indicates a predisposition to at least one of said NFkB associated disorders.

11. A method of diagnosing an NFkB associated condition or a susceptibility to a NFkB associated condition in a subject wherein said condition is a member of the group consisting of an immune disorder; an inflammatory disorder in which polypeptides of the present invention are associated with the disorder either directly, or indirectly; an inflammatory disorder related to aberrant NFkB regulation; a cancer; aberrant apoptosis; hepatic disorders; Hodgkins lymphomas; hematopoietic tumors; hyper-IgM syndromes; hypohydrotic ectodermal dysplasia; X-linked anhidrotic ectodermal dysplasia; Immunodeficiency; al incontinentia pigmenti; viral infections; HIV-1; HTLV-1; hepatitis B; hepatitis C; EBV; influenza; viral replication; host cell survival; and evasion of immune responses; rheumatoid arthritis inflammatory bowel disease; colitis; asthma; atherosclerosis; cachexia; euthyroid sick syndrome; stroke; EAE; autoimmune disorders; disorders related to hyper immune activity; disorders related to aberrant acute phase responses; hypercongenital conditions; birth defects; necrotic lesions; wounds; organ transplant rejection; conditions related to organ transplant rejection; disorders related to aberrant signal transduction; proliferating disorders; cancers; HIV propagation in cells infected with other viruses, associated with IL-8, disorders associated with aberrant IL-8 expression, disorders associated with aberrant IL-8 activity, asthma, pulmonary disorders, pulmonary fibrosis, Behcet's disease, bacterial infections, viral infections, gynaecological diseases, psoriasis, inflammatory bowel disease, IgA nephropathy, chronic obstructive pulmonary disease, Kawasaki disease, Crohn's disease, peripheral arterial occlusive disease, Hodgkin's disease, idiopathic intermediate uveitis, hyaline membrane disease, acute rheumatic fever, chronic rheumatic heart disease, ulcerative colitis, autoimmune disorders, and autoimmune thyroid disease, comprising:
(a) determining the presence or amount of expression of the polypeptide of claim 6 in a biological sample; and
(b) diagnosing a NFkB associated condition or a susceptibility to a pathological condition based on the presence or amount of expression of the polypeptide.

12. A method for identifying a binding partner to the polypeptide of claim 6 comprising:
(a) contacting the polypeptide of claim 6 with a binding partner; and
(b) determining whether the binding partner effects an activity of the polypeptide.

13. The method for preventing, treating, or ameliorating a medical condition of claim 9, wherein the medical condition is a member of the group consisting of an immune disorder; an inflammatory disorder in which polypeptides of the present invention are associated with the disorder either directly; or indirectly; an inflammatory disorder related to aberrant NFkB regulation; a cancer; aberrant apoptosis; hepatic disorders; Hodgkins lymphomas; hematopoietic tumors; hyper-IgM syndromes; hypohydrotic ectodermal dysplasia; X-linked anhidrotic ectodermal dysplasia; Immunodeficiency; al incontinentia pigmenti; viral infections; HIV-1; HTLV-1; hepatitis B; hepatitis C; EBV; influenza; viral replication; host cell survival; and evasion of immune responses; rheumatoid arthritis inflammatory bowel disease; colitis; asthma; atherosclerosis; cachexia; euthyroid sick syndrome; stroke; EAE; autoimmune disorders; disorders related to hyper immune activity; disorders related to aberrant acute phase responses; hypercongenital conditions; birth defects; necrotic lesions; wounds; organ transplant rejection; conditions related to organ transplant rejection; disorders related to aberrant signal transduction; proliferating disorders; cancers; HIV; propagation in cells infected with other viruses, associated with IL-8, disorders associated with aberrant IL-8 expression, disorders associated with aberrant IL-8 activity, asthma, pulmonary disorders, pulmonary fibrosis, Behcet's disease, bacterial infections, viral infections, gynaecological diseases, psoriasis, inflammatory bowel disease, IgA nephropathy, chronic obstructive pulmonary disease, Kawasaki disease, Crohn's disease, peripheral arterial occlusive disease, Hodgkin's disease, idiopathic intermediate uveitis, hyaline membrane disease, acute rheumatic fever, chronic rheumatic heart disease, ulcerative colitis, autoimmune disorders, and autoimmune thyroid disease..

14. A method of identifying a compound that modulates the biological activity of a NFkB associated molecule, comprising:
(a) combining a candidate modulator compound with a NFkB associated molecule having the sequence set forth in a member of the group consisting of SEQ ID NO:109-118, 126, 128, 144-152, or 160-161, or a polypeptide encoded by a polynucleotide selected from the group consisting of SEQ ID NO:1-108, 125, 127, 132-140, 158-159, or 264-284 ; and
(b) measuring an effect of the candidate modulator compound on the activity of the NFkB associated molecule.

15. A method of identifying a compound that modulates the biological activity of an NFkB associated molecule, comprising:
(a) combining a candidate modulator compound with a host cell expressing a NFkB associated molecule having the sequence as set forth in a member of the group consisting of SEQ ID NO:109-118, 126, 128, 144-152, or 160-161, or a polypeptide encoded by a polynucleotide selected from the group consisting of SEQ ID NO:1-108, 125, 127, 132-140, 158-159, or 264-284; and
(b) measuring an effect of the candidate modulator compound on the activity of the expressed NFkB associated molecule.

16. A method of identifying a compound that modulates the biological activity of a NFkB associated molecule, comprising:
(a) combining a candidate modulator compound with a host cell containing a vector comprising the polynucleotide sequence selected from the group consisting of SEQ ID NO:1-108, 125, 127, 132-140, 158-159, or 264-284, wherein a NFkB associated molecule is expressed by the cell; and
(b) measuring an effect of the candidate modulator compound on the activity of the expressed NFkB associated molecule.

17. A method of screening for a compound that is capable of modulating the biological activity of a NFkB associated molecule, comprising the steps of:
(a) providing a host cell containing a vector comprising the polynucleotide sequence selected from the group consisting of SEQ ID NO:1-108, 125, 127, 132-140, 158-159, or 264-284;
(b) determining the biological activity of the NFKB associated molecule in the absence of a modulator compound;
(c) contacting the cell with the modulator compound; and
(d) determining the biological activity of the NFKB associated molecule in the presence of the modulator compound; wherein a difference between the activity of the NFkB associated molecule in the presence of the modulator compound and in the absence of the modulator compound indicates a modulating effect of the compound.

18. A compound that modulates the biological activity of a human NFkB associated molecule as identified by the method according to a member of the group consisting of: the compound(s) identified according to the method of claim 14; the compound(s) identified according to the method of claim 15; the compound(s) identified according to the method of claim 16; and the compound(s) identified according to the method of claim 17.

19. The method of claim 10 further comprising the use of probes or primer pairs specific to a member of the group consisting of: (i) a polynucleotide encoding a polypeptide fragment of a member of the group consisting of SEQ ID NO:109-118, 126, 128, 144-152, 160, and 161; (ii) a polynucleotide encoding a polypeptide domain of a member of the group consisting of SEQ ID NO:109-118, 126, 128, 144-152, 160, and 161; (iii) a polynucleotide encoding a polypeptide epitope of a member of the group consisting of SEQ ID NO:109-118, 126, 128, 144-152, 160, and 161; (iv) a polynucleotide encoding a polypeptide of a member of the group consisting of SEQ ID NO:109-118, 126, 128, 144-152, 160, and 161 having NFkB modulating activity; (v) a polynucleotide encoding a polypeptide of a member of the group consisting of SEQ ID NO:109-118, 126, 128, 144-152, 160, and 161 which is modulated by NFkB or the NFkB pathway; (vi) a polynucleotide which represents the complimentary sequence (antisense) of a member of the group consisting of SEQ ID NO:1-108, 125, 127, 132-140, 158-159, and 264-284; (vii) a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues; (viii) an isolated nucleic acid molecule of a member of the group consisting of SEQ ID NO:109-118, 126, 128, 144-152, 160, and 161, wherein the polynucleotide fragment comprises a nucleotide sequence encoding a NFkB associated protein; (ix) an isolated nucleic acid molecule of a member of the group consisting of SEQ ID NO:1-1 08, 125, 127, 132-140, 158-159, and 264-284, wherein the polynucleotide fragment comprises a nucleotide sequence encoding the sequence identified as a member of the group consisting of SEQ ID NO:109-118, 126, 128, 144-152, 160, and 161, which is hybridizable to SEQ ID NO:1-108, 125, 127, 132-140, 158-159, and 264-284; and (x) an isolated nucleic acid molecule of a member of the group consisting of SEQ ID NO:1-108, 125, 127, 132-140, 158-159, and 264-284, wherein the polynucleotide fragment comprises the entire nucleotide sequence of a member of the group consisting of SEQ ID NO:1-108, 125, 127, 132-140, 158-159, and 264-284; wherein said method comprises the step of using said probe or primer pair to correlate expression of said member to a disease or disorder associated with said member.

20. The method of claim 11 comprising an antibody directed against a member of the group consisting of: SEQ ID NO:109-118, 126, 128, 144-152, 160, and 161, or encoded by the polynucleotide selected from the group consisting of SEQ ID NO:1-108, 125, 127, 132-140, 158-159, and 264-284.
